(19) United States
(12) Patent Application Publication
(10) Pub. No.: US 2007/0116726 A1
(43) Pub. Date: May 24, 2007

(54) Immunisation against Chlamydia pneumoniae

(75) Inventors: Guilio Ratti, Siena (IT); Guido Grandi, Siena (IT)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(73) Assignee: Chiron S.r.I., Siena (IT)

(21) Appl. No.: 11/414,403

(22) Filed: May 1, 2006

Related U.S. Application Data
(63) Continuation of application No. 10/312,273, filed on May 5, 2003, now abandoned.

(30) Foreign Application Priority Data
Jul. 3, 2000 (GB) 0016363.4
Jul. 11, 2000 (GB) 0017047.2
Jul. 21, 2000 (GB) 0017983.8
Aug. 7, 2000 (GB) 0019368.0
Aug. 18, 2000 (GB) 0020440.4
Sep. 14, 2000 (GB) 0022583.9
Nov. 10, 2000 (GB) 0027549.5
Dec. 22, 2000 (GB) 0031706.5

(51) Int. Cl. A61K 39/118; C07H 21/04; C12N 15/74; C12N 1/21; C07K 14/295

(52) U.S. Cl. 424/263.1; 435/69.3; 435/252.3; 435/471; 530/350; 536/23.7

(57) ABSTRACT

The published genomic sequence of Chlamydia pneumoniae reveals over 1000 putative encoded proteins but does not itself indicate which of these might be useful antigens for immunisation and vaccination or for diagnosis. This difficulty is addressed by the invention, which provides a number of C. pneumoniae protein sequences suitable for vaccine production and development and/or for diagnostic purposes.

This application is a continuation application of and claims priority to U.S. Ser. No. 10/312,273, filed May 5, 2003, which is a National Phase application of International Application No. PCT/IB01/01445, filed Jul. 3, 2001, all of which are incorporated herein in their entireties. All documents cited herein are incorporated by reference in their entirety.

### TECHNICAL FIELD

This invention is in the field of immunisation against chlamydial infection, in particular against infection by Chlamydia pneumoniae.

### BACKGROUND ART

Chlamydiae are obligate intracellular parasites of eukaryotic cells which are responsible for endemic sexually transmitted infections and various other disease syndromes. They occupy an exclusive eubacterial phylogenic branch, having no close relationship to any other known organisms—they are classified in their own order (Chlamydiales) which contains a single family (Chlamydiaceae) which in turn contains a single genus (Chlamydia). A particular characteristic of the Chlamydiae is their unique life cycle, in which the bacterium alternates between two morphologically distinct forms: an extracellular infective form (elementary bodies, EB) and an intracellular non-infective form (reticulate bodies, RB). The life cycle is completed with the re-organization of RB into EB, which subsequently leave the disrupted host cell ready to infect further cells.

Four chlamydial species are currently known—C. trachomatis, C. pneumoniae, C. pecorum and C. psittaci [e.g. Raulston (1995) Mol Microbiol 15:607-616; Everett (2000) Vet Microbiol 75:109-126]. C. pneumoniae is closely related to C. trachomatis, as the whole genome comparison of at least two isolates from each species has shown [Kalman et al. (1999) Nature Genetics 21:385-389; Read et al. (2000) Nucleic Acids Res 28:1397-406; Stephens et al. (1998) Science 282:754-759]. Based on surface reaction with patient immune sera, the current view is that only one serotype of C. pneumoniae exists world-wide.

C. pneumoniae is a common cause of human respiratory disease. It was first isolated from the conjunctiva of a child in Taiwan in 1965, and was established as a major respiratory pathogen in 1983. In the USA, C. pneumoniae causes approximately 10% of community-acquired pneumonia and 5% of pharyngitis, bronchitis, and sinusitis.

More recently, the spectrum of C. pneumoniae infections has been extended to include atherosclerosis, coronary heart disease, carotid artery stenosis, myocardial infarction, cerebrovascular disease, aortic aneurysm, claudication, and stroke. The association of C. pneumoniae with atherosclerosis is corroborated by the presence of the organism in atherosclerotic lesions throughout the arterial tree and the near absence of the organism in healthy arterial tissue. C. pneumoniae has also been isolated from coronary and carotid atheromatous plaques. The bacterium has also been associated with other acute and chronic respiratory diseases (e.g. otitis media, chronic obstructive pulmonary disease, pulmonary exacerbation of cystic fibrosis) as a result of sero-epidemiologic observations, case reports, isolation or direct detection of the organism in specimens, and successful response to anti-chlamydial antibiotics. To determine whether chronic infection plays a role in initiation or progression of disease, intervention studies in humans have been initiated, and animal models of C. pneumoniae infection have been developed.

Considerable knowledge of the epidemiology of C. pneumoniae infection has been derived from serologic studies using the C. pneumoniae-specific microimmunofluorescence test. Infection is ubiquitous, and it is estimated that virtually everyone is infected at some point in life, with common re-infection. Antibodies against C. pneumoniae are rare in children under the age of 5, except in developing and tropical countries. Antibody prevalence increases rapidly at ages 5 to 14, reaching 50% at the age of 20, and continuing to increase slowly to ˜80% by age 70.

A current hypothesis is that C. pneumoniae can persist in an asymptomatic low-grade infection in very large sections of the human population. When this condition occurs, it believed that the presence of C. pneumoniae, and/or the effects of the host reaction to the bacterium, can cause or help progress of cardiovascular illness.

It is not yet clear whether C. pneumoniae is actually a causative agent of cardiovascular disease, or whether it is just artefactually associated with it. It has been shown, however, that C. pneumoniae infection can induce LDL oxidation by human monocytes [Kalayoglu et al. (1999) J. Infect. Dis. 180:780-90; Kalayoglu et al. (1999) Am. Heart J. 138:S488-490]. As LDL oxidation products are highly atherogenic, this observation provides a possible mechanism whereby C. pneumoniae may cause atheromatous degeneration. If a causative effect is confirmed, vaccination (prophylactic and therapeutic) will be universally recommended.

Genomic sequence information has been published for C. pneumoniae [Kalman et al. (1999) supra; Read et al. (2000) supra; Shirai et al. (2000) J. Infect. Dis. 181(Suppl 3):S524-S527; WO99/27105; WO00/27994] and is available from GenBank. Sequencing efforts have not, however, focused on vaccination, and the availability of genomic sequence does not in itself indicate which of the >1000 genes might encode useful antigens for immunisation and vaccination. WO99/27105, for instance, implies that every one of the 1296 ORFs identified in the C. pneumoniae strain CM1 genome is a useful vaccine antigen.

It is thus an object of the present invention to identify antigens useful for vaccine production and development from amongst the many proteins present in C. pneumoniae. It is a further object to identify antigens useful for diagnosis (e.g. immunodiagnosis) of C. pneumoniae.

### DISCLOSURE OF THE INVENTION

The invention provides proteins comprising the C. pneumoniae amino acid sequences disclosed in the examples.

It also provides proteins comprising sequences which share at least x% sequence identity with the C. pneumoniae amino acid sequences disclosed in the examples. Depending on the particular sequence, x is preferably 50% or more (e.g. 60%, 70%, 80%, 90%, 95%, 99% or more). These include mutants and allelic variants. Typically, 50% identity or more between two proteins is considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1.

The invention further provides proteins comprising fragments of the C. pneumoniae amino acid sequences disclosed in the examples. The fragments should comprise at least n consecutive amino acids from the sequences and, depending on the particular sequence, n is 7 or more (e.g. 8, 10, 12, 14, 16, 18, 20, 30, 40, 50, 75, 100 or more). Preferably the fragments comprise one or more epitope(s) from the sequence. Other preferred fragments omit a signal peptide.

The proteins of the invention can, of course, be prepared by various means (e.g. native expression, recombinant expression, purification from cell culture, chemical synthesis etc.) and in various forms (e.g. native, fusions etc.). They are preferably prepared in substantially pure form (ie. substantially free from other C. pneumoniae or host cell proteins). Heterologous expression in E. coli is a preferred preparative route.

According to a further aspect, the invention provides nucleic acid comprising the C. pneumoniae nucleotide sequences disclosed in the examples. In addition, the invention provides nucleic acid comprising sequences which share at least x% sequence identity with the C. pneumoniae nucleotide sequences disclosed in the examples. Depending on the particular sequence, x is preferably 50% or more (e.g. 60%, 70%, 80%, 90%, 95%, 99% or more).

Furthermore, the invention provides nucleic acid which can hybridise to the C. pneumoniae nucleic acid disclosed in the examples, preferably under “high stringency” conditions (e.g. 65° C. in a 0.1×SSC, 0.5% SDS solution).

Nucleic acid comprising fragments of these sequences are also provided. These should comprise at least n consecutive nucleotides from the C. pneumoniae sequences and, depending on the particular sequence, n is 10 or more (e.g. 12, 14, 15, 18, 20, 25, 30, 35, 40, 50, 75, 100, 200, 300 or more).

According to a further aspect, the invention provides nucleic acid encoding the proteins and protein fragments of the invention.

It should also be appreciated that the invention provides nucleic acid comprising sequences complementary to those described above (e.g. for antisense or probing purposes).

Nucleic acid according to the invention can, of course, be prepared in many ways (e.g. by chemical synthesis, from genomic or cDNA libraries, from the organism itself etc.) and can take various forms (e.g. single stranded, double stranded, vectors, probes etc.).

In addition, the term “nucleic acid” includes DNA and RNA, and also their analogues, such as those containing modified backbones, and also peptide nucleic acids (PNA) etc.

According to a further aspect, the invention provides vectors comprising nucleotide sequences of the invention (e.g. cloning or expression vectors) and host cells transformed therewith.

According to a further aspect, the invention provides immunogenic compositions comprising protein and/or nucleic acid according to the invention. These compositions are suitable for immunisation and vaccination purposes. Vaccines of the invention may be prophylactic or therapeutic, and will typically comprise an antigen which can induce antibodies capable of inhibiting (a) chlamydial adhesion, (b) chlamydial entry, and/or (c) successful replication within the host cell. The vaccines preferably induce any cell-mediated T-cell responses which are necessary for chlamydial clearance from the host.

The invention also provides nucleic acid or protein according to the invention for use as medicaments (e.g. as vaccines). It also provides the use of nucleic acid or protein according to the invention in the manufacture of a medicament (e.g. a vaccine or an immunogenic composition) for treating or preventing infection due to C. pneumoniae.

The invention also provides a method of treating (e.g. immunising) a patient, comprising administering to the patient a therapeutically effective amount of nucleic acid or protein according to the invention.

According to further aspects, the invention provides various processes.

A process for producing proteins of the invention is provided, comprising the step of culturing a host cell according to the invention under conditions which induce protein expression.

A process for producing protein or nucleic acid of the invention is provided, wherein the protein or nucleic acid is synthesised in part or in whole using chemical means.

A process for detecting C. pneumoniae in a sample is provided, wherein the sample is contacted with an antibody which binds to a protein of the invention.

A summary of standard techniques and procedures which may be employed in order to perform the invention (e.g. to utilise the disclosed sequences for immunisation) follows. This summary is not a limitation on the invention but, rather, gives examples that may be used, but are not required.

General

The practice of the present invention will employ, unless otherwise indicated, conventional techniques of molecular biology, microbiology, recombinant DNA, and immunology, which are within the skill of the art. Such techniques are explained fully in the literature e.g. Sambrook Molecular Cloning, A Laboratory Manual, Second Edition (1989) and Third Edition (2001); DNA Cloning, Volumes I and ii (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification. Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional to X, such as X+Y.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Chlamydial sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (e.g. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (e.g. see U.S. Pat. No. 5,753,235).

Expression Systems

The Chlamydial nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning. A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236.1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982) PNAS USA 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning. A Laboratory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene-mediated transfection, protoplast fusion, electroporation, encapsulation of polynucleotide(s) in liposomes, direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between ˜1% and ˜5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers & Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g. Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g. HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038(1990); Maas et al., EMBO J. 9:3447-3452 (1990); Benkel and Hickey, Proc. Natl. Acad. Sci. 84:1337-1339 (1987)

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl. Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed. R. F. Goldberger)]. To express eukaryotic genes and prokaryotic genes with weak ribosome-binding site [Sambrook et al. (1989) “Expression of cloned genes in Escherichia coli.” In Molecular Cloning. A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann et al. (1985) Gene 40:183; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See e.g. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80: 1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g. EP-A-0 196 056. Another example is a ubiquitin fusion protein. Such a fusion protein is made with the ubiquitin region that preferably retains a site for a processing enzyme (e.g. ubiquitin-specific processing protease) to cleave the ubiquitin from the foreign protein. Through this method, therefore, native foreign protein can be isolated (e.g. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the genes for invertase (EP-A-0012873; JPO 62,096,086) and A-factor (U.S. Pat. No. 4,588,684). Alternatively, leaders of non-yeast origin exit, such as an interferon leader, that also provide for secretion in yeast (EP-A-0060057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (e.g. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pCl/1 [Brake et al. (1984) Proc. Natl. Acad. Sci USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See e.g. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et al., supra. One or more expression construct may integrate, possibly affecting levels of recombinant protein produced [Rine et al. (1983) Proc. Natl. Acad. Sci. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See e.g. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 & 4,929,555; Pichia]; [Hinnen et al. (1978) Proc. Natl. Acad. Sci. USA 75; 1929; Ito et al. (1983) J. Bacteriol. 153:163 Saccharomyces]; [Beach & Nurse (1981) Nature 300:706; Schizosaccharomyces]; [Davidow et al. (1985) Curr. Genet. 10:39; Gaillardin et al. (1985) Curr. Genet. 10:49; Yarrowia].

Pharmaceutical Compositions

Pharmaceutical compositions can comprise polypeptides and/or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Vaccines

Vaccines according to the invention may either be prophylactic (ie. to prevent infection) or therapeutic (ie. to treat disease after infection).

Such vaccines comprise immunising antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59™ (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (e.g. IL-1, IL-2, IL-4, IL-5, IL-6, IL-7, IL-12, etc.), interferons (e.g. gamma interferon), macrophage colony stimulating factor (M-CSF), tumor necrosis factor (TNF), etc; and (6) other substances that act as immunostimulating agents to enhance the effectiveness of the composition. Alum and MF59™ are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE), etc.

The immunogenic compositions (e.g. the immunising antigen/immunogen/polypeptide/protein/nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, e.g. by injection, either subcutaneously, intramuscularly, or transdermally/transcutaneously (e.g. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be employed [e.g. Robinson & Torres (1997) Seminars in Immunology 9:271-283; Donnelly et al. (1997) Annu Rev Immunol 15:617-648; see later herein].

Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses e.g. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J. Virol 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983)Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (ie. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar), pHSVlac described in Geller (1988) Science 241:1667-1669 and in WO90/09441 & WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with ATCC as accession numbers ATCC VR-977 and ATCC VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679,640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; ONyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W.H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

Delivery Methods

Once formulated, the polynucleotide compositions of the invention can be administered (1) directly to the subject; (2) delivered ex vivo, to cells derived from the subject; or (3) in vitro for recombinant protein expression. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Methods for the ex vivo delivery and reimplantation of transformed cells into a subject are known in the art and described in e.g. WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, etc.

Other groups that can be included are, for example: hormones, steroids, androgens, estrogens, thyroid hormone, or vitamins, folic acid.

C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, e.g. Szoka (1978) Proc. Natl. Acad. Sci. USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C, & E, over time these lipoproteins lose A and acquire C and E apoproteins. VLDL comprises A, B, C, & E apoproteins, LDL comprises apoprotein B; HDL comprises apoproteins A, C, & E.

The amino acid of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J. Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in Zuckermann et al. PCT/US97/14465.

F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] vol. 2, chapt. 9, pp. 9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 g for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of 24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:
Tm=81+16.6(log₁₀Ci)+0.4[%(G+C)]−0.6(%formamide)−600/n−1.5(%mismatch).
where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (ie. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Chlamydial nucleotide sequences of the invention (including both sense and antisense strands). Though many different nucleotide sequences will encode the amino acid sequence, the native Chlamydial sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Chlamydial sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Chlamydial sequence may also be helpful as a label to detect the formed duplex. For example, a non-complementary nucleotide sequence may be attached to the 5′ end of the probe, with the remainder of the probe sequence being complementary to a Chlamydial sequence. Alternatively, non-complementary bases or longer sequences can be interspersed into the probe, provided that the probe sequence has sufficient complementarity with the a Chlamydial sequence in order to hybridize therewith and thereby form a duplex which can be detected.

The exact length and sequence of the probe will depend on the hybridization conditions, such as temperature, salt condition and the like. For example, for diagnostic applications, depending on the complexity of the analyte sequence, the nucleic acid probe typically contains at least 10-20 nucleotides, preferably 15-25, and more preferably ≧30 nucleotides, although it may be shorter than this. Short primers generally require cooler temperatures to form sufficiently stable hybrid complexes with the template.

Probes may be produced by synthetic procedures, such as the triester method of Matteucci et al. [J. Am. Chem. Soc. (1981) 103:3185], or according to Urdea et al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated e.g. backbone modifications, such as phosphorothioates or methylphosphonates, can be used to increase in vivo half-life, alter RNA affinity, increase nuclease resistance etc. [e.g. see Agrawal & Iyer (1995) Curr Opin Biotechnol 6:12-19; Agrawal (1996) TIBTECH 14:376-387]; analogues such as peptide nucleic acids may also be used [e.g. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acids. The assay is described in: Mullis et al. [Meth. Enzymol. (1987) 155: 335-350]; U.S. Pat. Nos. 4,683,195 & 4,683,202. Two ‘primers’ hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Chlamydial sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labelled probe will hybridize to the Chlamydial sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### BRIEF DESCRIPTION OF THE DRAWINGS

FIGS. 1A-1C, 2A-2C, 3A-3C, 4A-4C, 5A-5C, 6A-6C, 7A-7C, 8A-8C, 9A-9C, 10A-10B, 11A-11C, 12A-12C, 13A-13B, 14A-14B, 15A-15C, 16A-16C, 17A-17C, 18A-18C, 19A-19B, 20A-20B, 21A-21C, 22A-22C, 23A-23C, 24A-24C, 25A-25C, 26A-26B, 27A-27C, 28A-28C, 29A-29C, 30A-30C, 31A-31B, 32A-32C, 33A-33B, 34A-34C, 35A-35C, 36A-36B, 37A-37D, 38A-38B, 39A-39D, 40A-40B, 41A-41C, 42A-42C, 43A-43C, 44A-44C, 45A-45C, 46A-46B, 47A-47C, 48A-48C, 49A-49C, 50A-50C, 51A-51C, 52A-52C, 53A-53B, 54A-54C, 55A-55C, 56A-56D, 57A-57C, 58A-58C, 59A-59C, 60A-60C, 61A-61C, 62A-62C, 63A-63C, 64A-64D, 65A-65C, 66A-66B, 67A-67B, 68A-68B, 69A-69B, 70A-70B, 71A-71B, 72A-72B, 73A-73B, 74A-74C, 75A-75B, 76A-76B, 77A-77B, 78A-78B, 79A-79B, 80A-80B, 81A-81B, 82A-82B, 83A-83B, 848A-84B, 85A-85B, 86A-86B, 87A-87B, 88A-88B, 89A-89B, 90A-90B, 91A-91B, 92A-92B, 93A-93C, 99A-99C, 95A-95C, 96A-96D, 97A-97C, 98A-98C, 99A-99C, 100A-100C, 101A-101C, 102A-102B, 103A-103C, 104A-104C, 105A-105B, 106A-106B, 107, 108A-108B, 109A-109B, 110A-110B, 111A-111B, 112A-112B, 113A-113B, 114A-114B, 115A-115B, 116A-116B, 117A-117B, 118A-118B, 119A-119B, 120A-120B, 121A-121B, 122A-122B, 123A-123B, 124A-124B, 125A-125B, 126A-126B, 127A-127B, 128A-128B, 129A-129B, 130A-130B, 131A-131B, 132A-132B, 133A-133B, 134A-134B, 135A-135B, 136A-136B, 137A-137B, 138A-138B, 139A-139B, 140A-140B, 141A-141B, 142A-142B, 143A-143B, 144A-144B, 145A-145B, 146A-146B, 147A-147B, 148A-148B, 149A-149B, 150A-150B, 151A-151B, 152A-152B, 153, 154A-154B, 155, 156, 157, 158, 159A-159B, 160, 161A-161B, 162, 163, 164A-164B, 165, 166, 167A-167B, 168, 169, 170, 171A-171B, 172, 173, 174A-174B, 175, 176, 177, 178, 179A-179B, 180A-180B, 181, 182, 183, 184, 185, 186A-186B, 187A-187B, 188A-188B, 189A-189B show data pertaining to examples 1-189, respectively.

FIG. 190 shows a representative 2D gel of proteins in elementary bodies.

FIG. 191 shows an alignment of sequences in five (six) proteins of the invention.

### EXAMPLES

The examples indicate C. pneumoniae proteins, together with evidence to support the view that the proteins are useful antigens for vaccine production and development or for diagnostic purposes. This evidence takes the form of:
Computer prediction based on sequence information from CWL029 strain (e.g. using the PSORT algorithm available from www.psort.nibb.ac.jp).
Data on recombinant expression and purification of the proteins cloned from IOL207 strain.
Western blots to demonstrate immunoreactivity in serum (typically a blot of an EB extract of C. pneumoniae strain FB/96 stained with mouse antiserum against the recombinant protein).
FACS analysis of C. pneumoniae bacteria or purified EBs to confirm accessibility of the antigen to the immune system (see also table III).
An indication if the protein was identified by MALDI-TOF from a 2D gel electrophoresis map of proteins from purified elementary bodies from strain FB/96. This confirms that the protein is expressed in vivo (see also table V).

Various tests can be used to assess the in vivo immunogenicity of the proteins identified in the examples. For example, the proteins can be expressed recombinantly and used to screen patient sera by immunoblot. A positive reaction between the protein and patient serum indicates that the patient has previously mounted an immune response to the protein in question ie. the protein is an immunogen. This method can also be used to identify immunodominant proteins.

The recombinant protein can also be conveniently used to prepare antibodies e.g. in a mouse. These can be used for direct confirmation that a protein is located on the cell-surface. Labelled antibody (e.g. fluorescent labelling for FACS) can be incubated with intact bacteria and the presence of label on the bacterial surface confirms the location of the protein.

In particular, the following methods (A) to (O) were used to express, purify and biochemically characterise the proteins of the invention:

Cloning of Cpn ORFs for Expression in E. coli

ORFs of Chlamydia pneumoniae (Cpn) were cloned in such a way as to potentially obtain three different kind of proteins:
a) proteins having an hexa-histidine tag at the C-terminus (cpn-His)
b) proteins having a GST fusion partner at the N-terminus (Gst-cpn)
c) proteins having both hexa-histidine tag at the C-terminus and GST at the N-terminus (GST/His fusion; NH₂-GST-cpn-(His)₆-COOH)

The type a) proteins were obtained upon cloning in the pET21b+ (Novagen). The type b) and c) proteins were obtained upon cloning in modified pGEX-KG vectors [Guan & Dixon (1991) Anal. Biochem. 192:262]. For instance pGEX-KG was modified to obtain pGEX-NN, then by modifying pGEX-NN to obtain pGEX-NNH. The Gst-cpn and Gst-cpn-His proteins were obtained in pGEX-NN and pGEX-NNH respectively.

The modified versions of pGEX-KG vector were made with the aim of allowing the cloning of single amplification products in all three vectors after only one double restriction enzyme digestion and to minimise the presence of extraneous amino acids in the final recombinant proteins.

(A) Construction of pGEX-NN and pGEX-NNH Expression Vectors

Two couples of complementary oligodeoxyribonucleotides were synthesised using the DNA synthesiser ABI394 (Perkin Elmer) and the reagents from Cruachem (Glasgow, Scotland). Equimolar amounts of the oligo pairs (50 ng each oligo) were annealed in T4 DNA ligase buffer (New England Biolabs) for 10 min in a final volume of 50 μl and then were left to cool slowly at room temperature. With the described procedure the following DNA linkers were obtained:

```
gexNN linker:
NdeI NheI XmaI EcoRI NcoI SalI XhoI SacI NotI
GATCCCATATGGCTAGCCCGGGGAATTCGTCCATGGAGTGAGTCGACTGACTCGAGTGATCGAGCTCCTGAGCGGCCGCATGAA
        GGTATACCGATCGGGCCCCTTAAGCAGGTACCTCACTCAGCTGACTGAGCTCACTAGCTCGAGGACTCGCCGGCGTACTTTCGA
gexNNH linker:
                      HindIII NotI XhoI --Hexa-Histidine--
            TCGACAAGCTTGCGGCCGCACTCGAGCATCACCATCACCATCACTGAT
                    GTTCGAACGCCGGCGTGAGCACGTAGAGGTAGTGGTAGTGACTATCGA
```

The plasmid pGEX-KG was digested with BamHI and HindIII and 100 ng were ligated overnight at 16° C. to the linker gexNN with a molar ratio of 3:1 linker/plasmid using 200 units of T4 DNA ligase (New england Biolabs). After transformation of the ligation product in E. coli DH5, a clone containing the pGEX-NN plasmid, having the correct linker, was selected by means of restriction enzyme analysis and DNA sequencing.

The new plasmid pGEX-NN was digested with SalI and HindIII and ligated to the linker gexNNH. After transformation of the ligation product in E. coli DH5, a clone containing the pGEX-NNH plasmid, having the correct linker, was selected by means of restriction enzyme analysis and DNA sequencing.

(B) Chromosomal DNA Preparation

The chromosomal DNA of elementary bodies (EB) of C. pneumoniae strain 10L-207 was prepared by adding 1.5 ml of lysis buffer (10 mM Tris-HCl, 150 mM NaCl, 2 mM EDTA, 0.6% SDS, 100 μg/ml Proteinase K, pH 8) to 450 μl EB suspension (400.000/μl) and incubating overnight at 37° C. After sequential extraction with phenol, phenol-chloroform, and chloroform, the DNA was precipitated with 0.3 M sodium acetate, pH 5.2 and 2 volumes of absolute ethanol. The DNA pellet was washed with 70% ethanol. After solubilization with distilled water and treatment with 20 μg/ml RNAse A for 1 hour at RT, the DNA was extracted again with phenol-chloroform, alcohol precipitated and suspended with 300 μl 1 mM Tris-HCl pH 8.5. The DNA concentration was evaluated by measuring OD₂₆₀ of the sample.

(C) Oligonucleotide Design

Synthetic oligonucleotide primers were designed on the basis of the coding sequence of each ORF using the sequence of C. pneumoniae strain CWL029. Any predicted signal peptide were omitted, by deducing the 5′ end amplification primer sequence immediately downstream from the predicted leader sequence. For most ORFs, the 5′ tail of the primers (table I) included only one restriction enzyme recognition site (NdeI, or NheI, or SpeI depending on the gene's own restriction pattern); the 3′ primer tails (table I) included a XhoI or a NotI or a HindIII restriction site.

| | |
|---|---|
| 5′ tails | 3′ tails |

| | | | |
|---|---|---|---|
| NdeI | 5′ GTGCGTCATATG 3′ | XhoI | 5′ GCGTCTCGAG 3′ |
| NheI | 5′ GTGCGTGCTAGC 3′ | NotI | 5′ |
| | | | ACTCGCTAGCGGCCGC 3′ |
| SpeI | 5′ GTGCGTACTAGT 3′ | HindIII | 5′ GCGTAAGCTT 3′ |

Table I. Oligonucleotide Tails of the Primers Used to Amplify Cpn Genes.

As well as containing the restriction enzyme recognition sequences, the primers included nucleotides which hybridized to the sequence to be amplified. The number of hybridizing nucleotides depended on the melting temperature of the primers which was determined as described [(Breslauer et al. (1986) PNAS USA 83:3746-50]. The average melting temperature of the selected oligos was 50-55° C. for the hybridizing region alone and 65-75° C. for the whole oligos. Table II shows the forward and reverse primers used for each amplification.

(D) Amplification

The standard PCR protocol was as follow: 50 ng genomic DNA were used as template in the presence of 0.2 μM each primer, 200 μM each dNTP, 1.5 mM MgCl₂, 1×PCR buffer minus Mg (Gibco-BRL), and 2 units of Taq DNA polymerase (Platinum Taq, Gibco-BRL) in a final volume of 100 μl. Each sample underwent a double-step amplification: the first 5 cycles were performed using as the hybridizing temperature the one of the oligos excluding the restriction enzyme tail, followed by 25 cycles performed according to the hybridization temperature of the whole length primers. The standard cycles were as follow:

| | | | |
|---|---|---|---|
| denaturation: | 94° C., 2 min | | |
| denaturation: | 94° C., 30 seconds | | |
| | | {close oversize brace} | 5 cycles |
| hybridization: | 51° C., 50 seconds | | |
| elongation: | 72° C., 1 min or 2 min and 40 sec | | |
| denaturation: | 94° C., 30 seconds | | |
| | | {close oversize brace} | 25 cycles |
| hybridization: | 70° C., 50 seconds | | |
| elongation: | 72° C., 1 min or 2 min and 40 sec | | |
| | 72° C., 7 min | | |
| | 4° C. | | |

The elongation time was 1 min for ORFs shorter than 2000 bp, and 2 min and 40 seconds for ORFs longer than 2000 bp. The amplifications were performed using a Gene Amp PCR system 9600 (Perkin Elmer).

To check the amplification results, 4 μl of each PCR product was loaded onto 1-1.5 agarose gel and the size of amplified fragments compared with DNA molecular weight standards (DNA markers III or IX, Roche). The PCR products were loaded on agarose gel and after electrophoresis the right size bands were excised from the gel. The DNA was purified from the agarose using the Gel Extraction Kit (Qiagen) following the instruction of the manufacturer. The final elution volume of the DNA was 50 μl TE (10 mM Tris-HCl, 1 mM EDTA, pH 8). One μl of each purified DNA was loaded onto agarose gel to evaluate the yield.

(E) Digestion of PCR Fragments

One-two μg of purified PCR product were double digested overnight at 37° C. with the appropriate restriction enzymes (60 units of each enzyme) using the appropriate restriction buffer in 100 μl final volume. The restriction enzymes and the digestion buffers were from New England Biolabs. After purification of the digested DNA (PCR purification Kit, Qiagen) and elution with 30 μl TE, 1 μl was subjected to agarose gel electrophoresis to evaluate the yield in comparison to titrated molecular weight standards (DNA markers III or IX, Roche).

(F) Digestion of the Cloning Vectors (pET21b+, pGEX-NN, and pGEX-NNH)

10 μg of plasmid was double digested with 100 units of each restriction enzyme in 400 μl reaction volume in the presence of appropriate buffer by overnight incubation at 37° C. After electrophoresis on a 1% agarose gel, the band corresponding to the digested vector was purified from the gel using the Qiagen Qiaex II Gel Extraction Kit and the DNA was eluted with 50 μl TE. The DNA concentration was evaluated by measuring OD₂₆₀ of the sample.

(G) Cloning

75 ng of the appropriately digested and purified vectors and the digested and purified fragments corresponding to each ORF, were ligated in final volumes of 10-20 μl with a molar ratio of 1:1 fragment/vector, using 400 units T4 DNA ligase (New England Biolabs) in the presence of the buffer supplied by the manufacturer. The reactions were incubated overnight at 16° C.

Transformation in E. coli DH5 competent cells was performed as follow: the ligation reaction was mixed with 200 μl of competent DH5 cells and incubated on ice for 30 min and then at 42° C. for 90 seconds. After cooling on ice, 0.8 ml LB was added and the cells were incubated for 45 min at 37° C. under shaking. 100 and 900 μl of cell suspensions were plated on separate plates of agar LB 100 μg/ml Ampicillin and the plates were incubated overnight at 37° C. The screening of the transformants was done by growing randomly chosen clones in 6 ml LB 100 μg/ml Ampicillin, by extracting the DNA using the Qiagen Qiaprep Spin Miniprep Kit following the manufacturer instructions, and by digesting 2 μl of plasmid minipreparation with the restriction enzymes specific for the restriction cloning sites. After agarose gel electrophoresis of the digested plasmid mini-preparations, positive clones were chosen on the basis of the correct size of the restriction fragments, as evaluated by comparison with appropriate molecular weight markers (DNA markers III or IX, Roche).

(H) Expression

1 μl of each right plasmid mini-preparation was transformed in 200 μl of competent E. coli strain suitable for expression of the recombinant protein. All pET21b+recombinant plasmids were transformed in BL21 DE3 (Novagen) E. coli cells, whilst all pGEX-NN and all pGEX-NNH recombinant plasmids were transformed in BL21 cells (Novagen). After plating transformation mixtures on LB/Amp agar plates and incubation overnight at 37° C., single colonies were inoculated in 3 ml LB 100 μg/ml Ampicillin and grown at 37° C. overnight. 70 μl of the overnight culture was inoculated in 2 ml LB/Amp and grown at 37° C. until OD₆₀₀ of the pET clones reached the 0.4-0.8 value or until OD₆₀₀ of the pGEX clones reached the 0.8-1 value. Protein expression was then induced by adding IPTG (Isopropil β-D thio-galacto-piranoside) to the mini-cultures. pET clones were induced using 1 mM IPTG, whilst pGEX clones were induced using 0.2 mM IPTG. After 3 hours incubation at 37° C. the final OD₆₀₀ was checked and the cultures were cooled on ice. After centrifugation of 0.5 ml culture, the cell pellet was suspended in 50 μl of protein Loading Sample Buffer (60 mM TRIS-HCl pH 6.8, 5% w/v SDS, 10% v/v glycerin, 0.1% w/v Bromophenol Blue, 100 mM DTT) and incubated at 100° C. for 5 min. A volume of boiled sample corresponding to 0.1 OD₆₀₀ culture was analysed by SDS-PAGE and Coomassie Blue staining to verify the presence of induced protein band.

Purification of the Recombinant Proteins

Single colonies were inoculated in 25 ml LB 100 μg/ml Ampicillin and grown at 37° C. overnight. The overnight culture was inoculated in 500 ml LB/Amp and grown under shaking at 25° C. until OD₆₀₀ 0.4-0.8 value for the pET clones, or until OD₆₀₀ 0.8-1 value for the pGEX clones. Protein expression was then induced by adding IPTG to the cultures. pET clones were induced using 1 mM IPTG, whilst pGEX clones were induced using 0.2 mM IPTG. After 4 hours incubation at 25° C. the final OD₆₀₀ was checked and the cultures were cooled on ice. After centrifugation at 6000 rpm (JA10 rotor, Beckman), the cell pellet was processed for purification or frozen at −20° C.

(I) Procedure for the Purification of Soluble His-Tagged Proteins from E. coli

1. Transfer the pellets from −20° C. to ice bath and reconstitute with 10 ml 50 mM NaHPO₄ buffer, 300 mM NaCl, pH 8.0, pass in 40-50 ml centrifugation tubes and break the cells as per the following outline:
2. Break the pellets in the French Press performing three passages with in-line washing.
3. Centrifuge at about 30-40000×g per 15-20 min. If possible use rotor JA 25.50 (21000 rpm, 15 min.) or JA-20 (18000 rpm, 15 min.)
4. Equilibrate the Poly-Prep columns with 1 ml Fast Flow Chelating Sepharose resin with 50 mM phosphate buffer, 300 mM NaCl, pH 8.0.
5. Store the centrifugation pellet at −20° C., and load the supernatant in the columns.
6. Collect the flow through.
7. Wash the columns with 10 ml (2 ml+2 ml+4 ml) 50 mM phosphate buffer, 300 mM NaCl, pH 8.0.
8. Wash again with 10 ml 20 mM imidazole buffer, 50 mM phosphate, 300 mM NaCl, pH 8.0.
9. Elute the proteins bound to the columns with 4.5 ml (1.5 ml+1.5 ml+1.5 ml) 250 mM imidazole buffer, 50 mM phosphate, 300 mM NaCl, pH 8.0 and collect the 3 corresponding fractions of 1.5 ml each. Add to each tube 15 μl DTT 200 mM (final concentration 2 mM)
10. Measure the protein concentration of the first two fractions with the Bradford method, collect a 10 μg aliquot of proteins from each sample and analyse by SDS-PAGE. (N.B.: should the sample be too diluted, load 21 μl+7 μl loading buffer).
11. Store the collected fractions at +4° C. while waiting for the results of the SDS-PAGE analysis.
12. For immunisation prepare 4-5 aliquots of 100 μg each in 0.5 ml in 40% glycerol. The dilution buffer is the above elution buffer, plus 2 mM DTT. Store the aliquots at −20° C. until immunisation.
   (J) Purification of His-Tagged Proteins from Inclusion Bodies

Purifications were carried out essentially according the following protocol:
1. Bacteria are collected from 500 ml cultures by centrifugation. If required store bacterial pellets at −20° C. For extraction, resuspend each bacterial pellet in 10 ml 50 mM TRIS-HCl buffer, pH 8.5 on an ice bath.
2. Disrupt the resuspended bacteria with a French Press, performing two passages.
3. Centrifuge at 35000×g for 15 min and collect the pellets. Use a Beckman rotor JA 25.50 (21000 rpm, 15 min.) or JA-20 (18000 rpm, 15 min.).
4. Dissolve the centrifugation pellets with 50 mM TRIS-HCl, 1 mM TCEP {Tris(2-carboxyethyl)-phosphine hydrochloride, Pierce}, 6M guanidium chloride, pH 8.5. Stir for ˜10 min. with a magnetic bar.
5. Centrifuge as described above, and collect the supernatant.
6. Prepare an adequate number of Poly-Prep (Bio-Rad) columns containing 1 ml of Fast Flow Chelating Sepharose (Pharmacia) saturated with Nichel according to manufacturer recommendations. Wash the columns twice with 5 ml of H₂0 and equilibrate with 50 mM TRIS-HCl, 1 mM TCEP, 6M guanidinium chloride, pH 8.5.
7. Load the supernatants from step 5 onto the columns, and wash with 5 ml of 50 mM TRIS-Hcl buffer, 1 mM TCEP, 6M urea, pH 8.5
8. Wash the columns with 10 ml of 20 mM imidazole, 50 mM TRIS-HCl, 6M urea, 1 mM TCEP, pH 8.5. Collect and set aside the first 5 ml for possible further controls.
9. Elute the proteins bound to the columns with 4.5 ml of a buffer containing 250 mM imidazole, 50 mM TRIS-HCl, 6M urea, 1 mM TCEP, pH 8.5. Add the elution buffer in three 1.5 ml aliquots, and collect the corresponding 3 fractions. Add to each fraction 15 μl DTT (final concentration 2 mM).
10. Measure eluted protein concentration with the Bradford method, and analyze aliquots of ca 10 μg of protein by SDS-PAGE.
11. Store proteins at −20° C. in 40% (v/v) glycerol, 50 mM TRIS-HCl, 2M urea, 0.5 M arginine, 2 mM DTT, 0.3 mM TCEP, 83.3 mM imidazole, pH 8.5
   (K) Procedure for the Purification of GST-Fusion Proteins from E. coli
1. Transfer the bacterial pellets from −20° C. to an ice bath and resuspend with 7.5 ml PBS, pH 7.4 to which a mixture of protease inhibitors (COMPLETE™—Boehringer Mannheim, 1 tablet every 25 ml of buffer) has been added. Transfer to 40-50 ml centrifugation tubes and sonicate according to the following procedure:
   a) Position the probe at about 0.5 cm from the bottom of the tube
   b) Block the tube with the clamp
   c) Dip the tube in an ice bath
   d) Set the sonicator as follows: Timer→Hold, Duty Cycle→55, Out. Control→6.
   e) perform 5 cycles of 10 impulses at a time lapse of 1 minute (i.e. one cycle=10 impulses+˜45″ hold; b. 10 impulses+˜45″ hold; c. 10 impulses+˜45″ hold; d. 10 impulses+˜45″ hold; e. 10 impulses+˜45″ hold)
2. Centrifuge at about 30-40000×g for 15-20 min. E.g.: use rotor Beckman JA 25.50 at 21000 rpm, for 15 min.
3. Store the centrifugation pellets at −20° C., and load the supernatants on the chromatography columns, as follows
4. Equilibrate the Poly-Prep (Bio-Rad) columns with 0.5 ml (≅1 ml suspension) of Glutathione-Sepharose 4B resin, wash with 2 ml (1+1) H₂O, and then with 10 ml (2+4+4) PBS, pH 7.4.
5. Load the supernatants on the columns and discard the flow through.
6. Wash the columns with 10 ml (2+4+4) PBS, pH 7.4.
7. Elute the proteins bound to the columns with 4.5 ml of 50 mM TRIS buffer, 10 mM reduced glutathione, pH 8.0, adding 1.5 ml+1.5 ml+1.5 ml and collecting the respective 3 fractions of ˜1.5 ml each.
8. Measure the protein concentration of the first two fractions with the Bradford method, analyse a 10 μg aliquot of proteins from each sample by SDS-PAGE. (N.B.: if the sample is too diluted load 21 μl (+7 μl loading buffer).
9. Store the collected fractions at +4° C. while waiting for the results of the SDS-PAGE analysis.
10. For each protein destined to the immunisation prepare 4-5 aliquots of 100 μg each in 0.5 ml of 40% glycerol. The dilution buffer is 50 mM TRIS.HCl, 2 mM DTT, pH 8.0. Store the aliquots at −20° C. until immunisation.
   Serology
   (L) Protocol of Immunization
1. Groups of four CD1 female mice aged between 6 and 7 weeks were immunized with 20 μg of recombinant protein resuspended in 100 μl.
2. Four mice for each group received 3 doses with a 14 days interval schedule.
3. Immunization was performed through intra-peritoneal injection of the protein with an equal volume of Complete Freund's Adjuvant (CFA) for the first dose and Incomplete Freund's Adjuvant (IFA) for the following two doses.
4. Sera were collected before each immunization. Mice were sacrified 14 days after the third immunization and the collected sera were pooled and stored at −20° C.
   (M) Western Blot Analysis of Cpn Elementary Body Proteins with Mouse Sera

Aliquots of elementary bodies containing approximately 4 μg of proteins, mixed with SDS loading buffer (1×: 60 mM TRIS-HCl pH 6.8, 5% w/v SDS, 10% v/v glycerin, 0.1% Bromophenol Blue, 100 mM DTT) and boiled 5 minutes at 95° C., were loaded on a 12% SDS-PAGE gel. The gel was run using a SDS-PAGE running buffer containing 250 mM TRIS, 2.5 mM Glycine and 0.1% SDS. The gel was electroblotted onto nitrocellulose membrane at 200 mA for 30 minutes. The membrane was blocked for 30 minutes with PBS, 3% skimmed milk powder and incubated O/N at 4° C. with the appropriate dilution (1/100) of the sera. After washing twice with PBS+0.1% Tween (Sigma) the membrane was incubated for 2 hours with peroxidase-conjugated secondary anti-mouse antibody (Sigma) diluted 1:3000. The nitrocellulose was washed twice for 10 minutes with PBS+0.1% Tween-20 and once with PBS and thereafter developed by Opti-4CN Substrate Kit (Biorad).

Lanes shown in Western blots are: (P)=pre-immune control serum; (I)=immune serum.

(N) FACS Analysis of Chlamydia pneumoniae Elementary Bodies with Mouse Sera

1. 2×10⁵ Elementary Bodies (EB)/well were washed with 200 μl of PBS-01% BSA in a 96 wells U bottom plate and centrifuged for 10 min. at 1200 rpm, at 4° C.
2. The supernatant was discarded and the E.B. resuspended in 10 μl of PBS-0.1% BSA.
3. 10 μl mouse sera diluted in PBS-01% BSA were added to the E.B. suspension to a final dilution of 1:400, and incubated on ice for 30 min.
4. EB were washed by adding 180 μl PBS-0.1% BSA and centrifuged for 10 min. at 1200 rpm, 4° C.
5. The supernatant was discarded and the E.B. resuspended in 10 l of PBS-0.1% BSA.
6. 10 μl of a goat anti-mouse IgG, F(ab′)₂ fragment specific-R-Phycoerythrin-conjugated (Jackson Immunoresearch Laboratories Inc., cat. No 115-116-072) was added to the EB suspension to a final dilution of 1:100, and incubated on ice for 30 min. in the dark.
7. EB were washed by adding 180 μl PBS-0.1% BSA and centrifuged for 10 min. at 1200 rpm, 4° C.
8. The supernatant was discarded and the E.B. resuspended in 150 μl of PBS-0.1% BSA.
9. E.B. suspension was passed through a cytometric chamber of a FACS Calibur (Becton Dikinson, Mountain View, Calif. USA) and 10.000 events were acquired.
10. Data were analysed using Cell Quest Software (Becton Dikinson, Mountain View, Calif. USA) by drawing a morphological dot plot (using forward and side scatter parameters) on E.B. signals. An histogram plot was then created on FL2 intensity of fluorescence log scale recalling the morphological region of EB.

NB: the results of FACS depend not only on the extent of accessibility of the native antigens but also on the quality of the antibodies elicited by the recombinant antigens, which may have structures with a variable degree of correct folding as compared with the native protein structures. Therefore, even if a FACS assay appears negative this does not necessarily mean that the protein is not abundant or accessible on the surface. PorB antigen, for instance, gave negative results in FACS but is a surface-exposed neutralising antigen [Kubo & Stephens (2000) Mol. Microbiol. 38:772-780].

(O) Mass Spectrometry Analysis of Two-Dimensional Electrophoretic Protein Maps

Gradient purified EBs from strain FB/96 were solubilized at a final concentration of 5.5 mg/ml with immobiline rehydratation buffer (7M urea, 2M thiourea, 2% (w/v) CHAPS, 2% (w/v) ASB 14 [Chevallet et al. (1998) Electrophor. 19:1901-9], 2% (v/v) C.A 3-10NL (Amersham Pharmacia Biotech), 2 mM tributyl phosphine, 65 mM DTT). Samples (250 μg protein) were adsorbed overnight on Immobiline DryStrips (7 cm, pH 3-10 non linear). Electrophocusing was performed in a IPGphor Isoelectric Focusing Unit (Amersham Pharmacia Biotech). Before PAGE separation, the focused strips were incubated in 4M urea, 2M thiourea, 30% (v/v) glycerol, 2% (w/v) SDS, 5 mM tributyl phosphine 2.5% (w/v) acrylamide, 50 mM Tris-HCl pH 8.8, as described [Herbert et al. (1998) Electrophor. 19:845-51]. SDS-PAGE was performed on linear 9-16% acrylamide gradients. Gels were stained with colloidal Coomassie (Novex, San Diego) [Doherty et al. (1998) Electrophor. 19:355-63]. Stained gels were scanned with a Personal Densitometer SI (Molecular Dynamics) at 8 bits and 50 μm per pixel. Map images were annotated with the software Image Master 2D Elite, version 3.10 (Amersham Pharmacia Biotech). Protein spots were excised from the gel, using an Ettan Spot picker (Amersham Pharmacia Biotech), and dried in a vacuum centrifuge. In-gel digestion of samples for mass spectrometry and extraction of peptides were performed as described by Wilm et al. [Nature (1996) 379:466-9]. Samples were desalted with a ZIP TIP (Millipore), eluted with a saturated solution of alpha-cyano-4-hydroxycinnamic acid in 50% acetonitrile, 0.1% TFA and directly loaded onto a SCOUT 381 multiprobe plate (Bruker). Spectra were acquired on a Bruker Biflex II MALDI-TOF. Spectra were calibrated using a combination of known standard peptides, located in spots adjacent to the samples. Resulting values for monoisotopic peaks were used for database searches using the computer program Mascot (www.matrixscience.com). All searches were performed using an error of 200-500 ppm as constraint. A representative gel is shown in FIG. 190.

### Example 1

The following C. pneumoniae protein (PID 4376552) was expressed <SEQ ID 1; cp6552>:

| | |
|---|---|
| 1 | MKKKLSLLVG LIFVLSSCHK EDAQNKIRIV ASPTPHAELL |
| | ESLQEEAKDL |
| 51 | GIKLKILPVD DYRIPNRLLL DKQVDANYFQ HQAFLDDECE |
| | RYDCKGELVV |
| 101 | IAKVHLEPQA IYSKKHSSLE RLKSQKKLTI AIPVDRTNAQ |
| | RALHLLEECG |
| 151 | LIVCKGPANL NMTAKDVCGK ENRSINILEV SAPLLVGSLP |
| | DVDAAVIPGN |
| 201 | FAIAANLSPK KDSLCLEDLS VSKYTNLVVI RSEDVGSPKM |
| | IKLQKLFQSP |
| 251 | SVQHFFDTKY HGNILTMTQD NG* |

A predicted signal peptide is highlighted.

The cp6552 nucleotide sequence <SEQ ID 2> is:

| | |
|---|---|
| 1 | ATGAAAAAAA AATTATCATT ACTTGTAGGT TTAATTTTTG |
| | TTTTGAGTTC |
| 51 | TTGCCATAAG GAAGATGCTC AGAATAAAAT ACGTATTGTA |
| | GCCAGTCCGA |
| 101 | CACCTCATGC GGAATTATTG GAGAGTTTAC AGGAAGAGGC |
| | TAAAGATCTT |
| 151 | GGAATCAAGC TGAAAATACT TCCAGTAGAT GATTATCGTA |
| | TTCCTAATCG |
| 201 | TTTGCTTTTG GATAAACAAG TAGATGCAAA TTACTTTCAA |
| | CATCAAGCTT |
| 251 | TTCTTGATGA CGAATGCGAG CGTTATGATT GTAAGGGTGA |
| | ATTAGTTGTT |
| 301 | ATCGCTAAAG TTCATTTGGA ACCTCAAGCA ATTTATTCTA |
| | AGAAACATTC |
| 351 | TTCTTTAGAG CGCTTAAAAA GCCAGAAGAA ACTGACTATA |
| | GCGATTCCTG |
| 401 | TGGATCGTAC GAATGCTCAG CGTGCTCTAC ACTTGTTAGA |
| | AGAGTGCGGA |
| 451 | CTCATTGTTT GCAAAGGGCC TGCTAATTTA AATATGACAG |
| | CTAAAGATGT |
| 501 | CTGTGGGAAA GAAAATAGAA GTATCAACAT ATTAGAGGTG |
| | TCAGCTCCTC |
| 551 | TTCTTGTCGG ATCTCTTCCT GACGTTGATG CTGCTGTCAT |
| | TCCTGGAAAT |
| 601 | TTTGCTATAG CAGCAAACCT TTCTCCAAAG AAAGATAGTC |
| | TTTGTTTAGA |
| 651 | GGATCTTTCG GTATCTAAGT ATACAAACCT TGTTGTCATT |
| | CGTTCTGAAG |
| 701 | ACGTAGGTTC TCCTAAAATG ATAAAATTAC AGAAGCTGTT |
| | TCAATCTCCT |
| 751 | TCTGTACAAC ATTTTTTTGA TACAAAATAT CATGGGAATA |
| | TTTTGACAAT |
| 801 | GACTCAAGAC AATGGTTAG |

The PSORT algorithm predicts an inner membrane location (0.127).

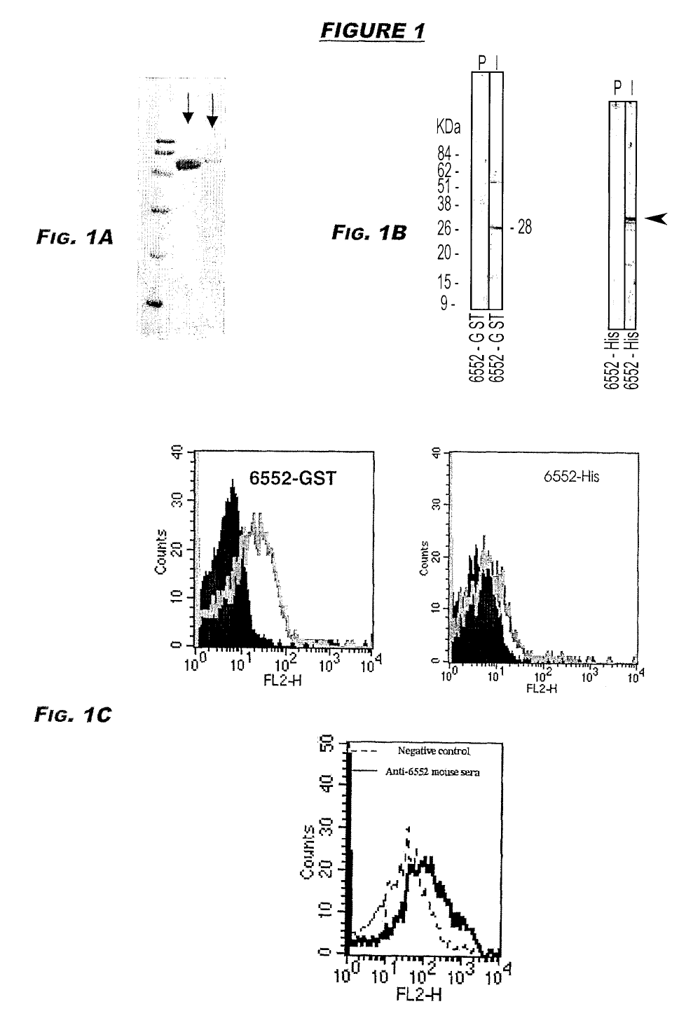

The protein was expressed in E. coli and purified as a his-tag product, as shown in FIG. 1A, and also as a GST-fusion. The recombinant protein was used to immunise mice, whose sera were used in a Western blot (FIG. 1B) and for FACS analysis (FIG. 1C).

The cp6552 protein was also identified in the 2D-PAGE experiment (Cpn0278).

These experiments show that cp6552 is a surface-exposed and immunoaccessible protein, and that it is a useful immunogen. These properties are not evident from the sequence alone.

### Example 2

The following C. pneumoniae protein (PID 4376736) was expressed <SEQ ID 3; cp6736>:

| | |
|---|---|
| 1 | MKTSIRKFLI STTLAPCFAS TAFTVEVIMP SENFDGSSGK |
| | IFRYTTLSDP |
| 51 | RGTLCIFSGD LYIANLDNAI SRTSSSCFSN RAGALQILGK |
| | GGVFSFLNIR |
| 101 | SSADGAAISS VITQNPELCP LSFSGFSQMI FDNCESLTSD |
| | TSASNVIPHA |
| 151 | SAIYATTPML FTNNDSILFQ YNRSAGFGAA IRGTSITIEN |
| | TKKSLLFNGN |
| 201 | GSISNGGALT GSAAINLINN SAPVIFSTNA TGIYGGAIYL |
| | TGGSMLTSGN |
| 251 | LSGVLFVNNS SRSGGAIYAN GNVTFSNNSD LTFQNNTASP |
| | QNSLPAPTPP |
| 301 | PTPPAVTPLL GYGGAIFCTP PATPPPTGVS LTISGENSVT |
| | FLENIASEQG |
| 351 | GALYGKKISI DSNKSTIFLG NTAGKGGAIA IPESGELSLS |
| | ANQGDILFNK |
| 401 | NLSITSGTPT RNSIHFGKDA KFATLGATQG YTLYFYDPIT |
| | SDDLSAASAA |
| 451 | ATVVVNPKAS ADGAYSGTIV FSGETLTATE AATPANATST |
| | LNQKLELEGG |
| 501 | TLALRNGATL NVHNFTQDEK SVVIMDAGTT LATTNGANNT |
| | DGAITLNKLV |
| 551 | INLDSLDGTK AAVVNVQSTN GALTISGTLG LVKNSQDCCD |
| | NHGMFNKDLQ |
| 601 | QVPILELKAT SNTVTTTDFS LGTNGYQQSP YGYQGTWEFT |
| | IDTTTHTVTG |
| 651 | NWKKTGYLPH PERLAPLIPN SLWANVIDLR AVSQASAADG |
| | EDVPGKQLSI |
| 701 | TGITNFFHAN HTGDARSYRH MGGGYLINTY TRITPDAALS |
| | LGFGQLFTKS |
| 751 | KDYLVGHGHS NVYFATVYSN ITKSLFGSSR FFSGGTSRVT |
| | YSRSNEKVKT |
| 801 | SYTKLPKGRC SWSNNCWLGE LEGNLPITLS SRILNLKQII |
| | PFVKAEVAYA |
| 851 | THGGIQENTP EGRIFGHGHL LNVAVPVGVR FGKNSHNRPD |
| | FYTIIVAYAP |
| 901 | DVYRHNPDCD TTLPINGATW TSIGNNLTRS TLLVQASSHT |
| | SVNDVLEIFG |
| 951 | HCGCDIRRTS RQYTIDIGSK LRF* |

A predicted signal peptide is highlighted.

The cp6736 nucleotide sequence <SEQ ID 4> is:

| | |
|---|---|
| 1 | ATGAAAACGT CTATTCGTAA GTTCTTAATT TCTACCACAC |
| | TGGCGCCATG |
| 51 | TTTTGCTTCA ACAGCGTTTA CTGTAGAAGT TATCATGCCT |
| | TCCGAGAACT |
| 101 | TTGATGGATC GAGTGGGAAG ATTTTTCCTT ACACAACACT |
| | TTCTGATCCT |
| 151 | AGAGGGACAC TCTGTATTTT TTCAGGGGAT CTCTACATTG |
| | CGAATCTTGA |
| 201 | TAATGCCATA TCCAGAACCT CTTCCACTTG CTTTAGCAAT |
| | AGGGCGGGAG |
| 251 | CACTACAAAT CTTAGGAAAA GGTGGGGTTT TCTCCTTCTT |
| | AAATATCCGT |
| 301 | TCTTCAGCTG ACGGAGCCGC GATTAGTAGT GTAATCACCC |
| | AAAATCCTGA |
| 351 | ACTATGTCCC TTGAGTTTTT CAGGATTTAG TCAGATGATC |
| | TTCGATAACT |
| 401 | GTGAATCTTT GACTTCAGAT ACCTCAGCGA GTAATGTCAT |
| | ACCTCACGCA |
| 451 | TCGGCGATTT ACGCTACAAC GCCCATGCTC TTTACAAACA |
| | ATGACTCCAT |
| 501 | ACTATTCCAA TACAACCGTT CTGCAGGATT TGGAGCTGCC |
| | ATTCGAGGCA |
| 551 | CAAGCATCAC AATAGAAAAT ACGAAAAAGA GCCTTCTCTT |
| | TAATGGTAAT |
| 601 | GGATCCATCT CTAATGGAGG GGCCCTCACG GGATCTGCAG |
| | CGATCAACCT |
| 651 | CATCAACAAT AGCGCTCCTG TGATTTTCTC AACGAATGCT |
| | ACAGGGATCT |
| 701 | ATGGTGGGGC TATTTACCTT ACCGGAGGAT CTATGCTCAC |
| | CTCTGGGAAC |
| 751 | CTCTCAGGAG TCTTGTTCGT TAATAATAGC TCGCGCTCAG |
| | GAGGCGCTAT |
| 801 | CTATGCTAAC GGAAATGTCA CATTTTCTAA TAACAGCGAC |
| | CTGACTTTCC |
| 851 | AAAACAATAC AGCATCTCCA CAAAACTCCT TACCTGCACC |
| | TACACCTCCA |
| 901 | CCTACACCAC CAGCAGTCAC TCCTTTGTTA GGATATGGAG |
| | GCGCCATCTT |
| 951 | CTGTACTCCT CCAGCTACCC CCCCACCAAC AGGTGTTAGC |
| | CTGACTATAT |
| 1001 | CTGGAGAAAA CAGCGTTACA TTCCTAGAAA ACATTGCCTC |
| | CGAACAAGGA |
| 1051 | GGAGCCCTCT ATGGCAAAAA GATCTCTATA GATTCTAATA |
| | AATCTACAAT |
| 1101 | ATTTCTTGGA AGTACAGCTG GAAAAGGAGG CGCTATTGCT |
| | ATTCCCGAAT |
| 1151 | CTGGGGAGCT CTCTCTATCC GCAAATCAAG GTGATATCCT |
| | CTTTAACAAG |
| 1201 | AACCTCAGCA TCACTAGTGG GACACCTACT CGCAATAGTA |
| | TTCACTTCGG |
| 1251 | AAAAGATGCC AAGTTTGCCA CTCTAGGAGC TACGCAAGGC |
| | TATACCCTAT |
| 1301 | ACTTCTATGA TCCGATTACA TCTGATGATT TATCTGCTGC |
| | ATCCGCAGCC |
| 1351 | GCTACTGTGG TCGTCAATCC CAAAGCCAGT GCAGATGGTG |
| | CGTATTCAGG |
| 1401 | GACTATTGTC TTTTCAGGAG AAACCCTCAC TGCTACCGAA |
| | GCAGCAACCC |
| 1451 | CTGCAAATGC TACATCTACA TTAAACCAAA AGCTAGAACT |
| | TGAAGGCGGT |
| 1501 | ACTCTCGCTT TAAGAAACGG TGCTACCTTA AATGTTCATA |
| | ACTTCACGCA |
| 1551 | AGATGAAAAG TCCGTCGTCA TCATGGATGC AGGGACCACA |
| | TTAGCAACTA |
| 1601 | CAAATGGAGC TAATAATACT GACGGTGCTA TCACCTTAAA |
| | CAAGCTTGTA |
| 1651 | ATCAATCTGG ATTCTTTGGA TGGCACTAAA GCGGCTGTCG |
| | TTAATGTGCA |
| 1701 | GAGTACCAAT GGAGCTCTCA CTATATCCGG AACTTTAGGA |
| | CTTGTGAAAA |
| 1751 | ACTCTCAAGA TTGCTGTGAC AACCACGGGA TGTTTAATAA |
| | AGATTTACAG |
| 1801 | CAAGTTCCGA TTTTAGAACT CAAAGCGACT TCAAATACTG |
| | TAACCACTAC |
| 1851 | GGACTTCAGT CTCGGCACAA ACGGCTATCA GCAATCTCCC |
| | TATGGGTATC |
| 1901 | AAGGAACTTG GGAGTTTACC ATAGACACGA CAACCCATAC |
| | GGTCACAGGA |
| 1951 | AATTGGAAAA AAACCGGTTA TCTTCCTCAT CCGGAGCGTC |
| | TTGCTCCCCT |
| 2001 | CATTCCTAAT AGCCTACGGG CAAACGTCAT AGATTTACGA |
| | GCTGTAAGTC |
| 2051 | AAGCGTCAGC AGCTGATGGC GAAGATGTCC CTGGGAAGCA |
| | ACTGAGCATC |
| 2101 | ACAGGAATTA CAAATTTCTT CCATGCGAAT CATACCGGTG |
| | ATGCACGCAG |
| 2151 | CTACCGCCAT ATGGGTGGAG GCTACCTCAT CAATACCTAC |
| | ACACGCATCA |
| 2201 | CTCCAGATGC TGCGTTAAGT CTAGGTTTTG GACAGCTGTT |
| | TACAAAATCT |
| 2251 | AAGGATTACC TCGTAGGTCA CGGTCATTCT AACGTTTATT |
| | TCGCTACAGT |
| 2301 | ATACTCTAAC ATCACCAAGT CTCTGTTTGG ATCATCGAGA |
| | TTCTTCTCAG |
| 2351 | GAGGCACTTC TCGAGTTACC TATAGCCGTA GCAATGAGAA |
| | AGTAAAGACT |
| 2401 | TCATATACAA AATTGCCTAA AGGGCGCTGC TCTTGGAGTA |
| | ACAATTGCTG |
| 2451 | GTTAGGAGAA CTCGAAGGGA ACCTTCCCAT CACTCTCTCT |
| | TCTCGCATCT |
| 2501 | TAAACCTCAA GCAGATCATT CCCTTTGTAA AAGCTGAAGT |
| | TGCTTACGCG |
| 2551 | ACTCATGGGG GCATCCAAGA AAATACCCCC GAGGGGAGGA |
| | TTTTTGGACA |
| 2601 | CGGTCATCTA CTCAACGTTG CAGTTCCCGT AGGCGTCCGC |
| | TTTGGTAAAA |
| 2651 | ATTCTCATAA TCGACCAGAT TTTTACACTA TAATCGTAGC |
| | CTATGCTCCT |
| 2701 | GATGTCTATC GTCACAATCC TGATTGCGAT ACGACATTAC |
| | CTATTAATGG |
| 2751 | AGCTACGTGG ACCTCTATAG GGAATAATCT AACCAGAAGT |
| | ACTTTGCTAG |
| 2801 | TACAAGCATC CAGCCATACT TCAGTAAATG ATGTTCTAGA |
| | GATCTTCGGG |
| 2851 | CACTGTGGAT GTGATATTCG CAGAACCTCC CGTCAATATA |
| | CTCTAGATAT |
| 2901 | AGGAAGCAAA TTACGATTTT AA |

The PSORT algorithm predicts an outer membrane location (0.917).

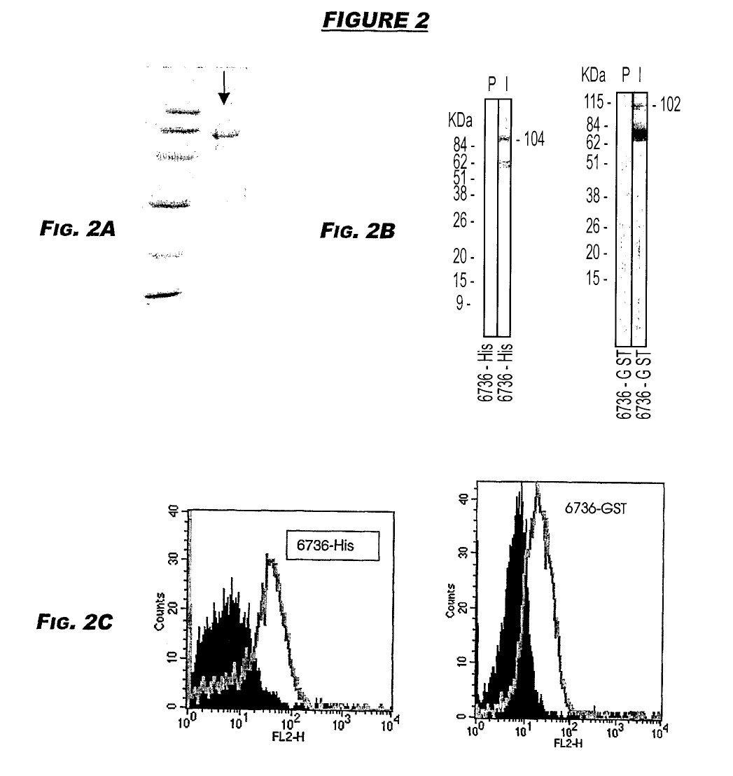

The protein was expressed in E. coli and purified as a his-tag product, as shown in FIG. 2A, and also as a GST-fusion. Both proteins were used to immunise mice, whose sera were used in a Western blot (FIG. 2B) and for FACS analysis (FIG. 2C).

The cp6736 protein was also identified in the 2D-PAGE experiment (Cpn0453) and showed good cross-reactivity with human sera, including sera from patients with pneumonitis.

These experiments show that cp6736 is a surface-exposed and immunoaccessible protein, and that it is a useful immunogen. These properties are not evident from the sequence alone.

### Example 3

The following C. pneumoniae protein (PID 4376751) was expressed <SEQ ID 5; cp6751>:

| | |
|---|---|
| 1 | MRFFCFGMLL PFTFVLANEG LQLPLETYIT LSPEYQAAPQ |
| | VGFTHNQNQD |
| 51 | LAIVGNHNDF ILDYKYYRSN GGALTCKNLL ISENIGNVFF |
| | EKNVCPNSGG |
| 101 | AIYAAQNCTI SKNQNYAFTT NLVSDNPTAT AGSLLGGALF |
| | AINCSITNNL |
| 151 | GQGTFVDNLA LNKGGALYTE TNLSIKDNKG PIIIKQNRAL |
| | NSDSLGGGIY |
| 201 | SGNSLNIEGN SGAIQITSNS SGSGGGIFST QTLTISSNKK |
| | LIEISENSAF |
| 251 | ANNYGSNFNP GGGGLTTTFC TILNNREGVL FNNNQSQSNG |
| | GAIHAKSIII |
| 301 | KENGPVYFLN NTATRGGALL NLSAGSGNGS FILSADNGDI |
| | IFNNNTASKH |
| 351 | ALNPPYRNAI HSTPNMNLQI GARPGYRVLF YDPIEHELPS |
| | SFPILFNFET |
| 401 | GHTGTVLFSG EHVHQNFTDE NMFFSYLRNT SELRQGVLAV |
| | EDGAGLACYK |
| 451 | FFQRGGTLLL GQGAVITTAG TIPTPSSTPT TVGSTITLNH |
| | IAIDLPSILS |
| 501 | FQAQAPKIWI YPTKTGSTYT EDSNPTITIS GTLTLRNSNN |
| | EDPYDSLDLS |
| 551 | HSLEKVPLLY IVDVAAQKIN SSQLDLSTLN SGEHYGYQGI |
| | WSTYWVETTT |
| 601 | ITNPTSLLGA NTKHKLLYAN WSPLGYRPHP ERRGEFITNA |
| | LWQSAYTALA |
| 651 | GLHSLSSWDE EKGHAASLQG IGLLVHQKDK NGFKGFRSHM |
| | TGYSATTEAT |
| 701 | SSQSPNFSLG FAQFFSKAKE HESQNSTSSH HYFSGMCIEN |
| | TLFKEWIRLS |
| 751 | VSLAYMFTSE HTHTMYQGLL EGNSQGSFHN HTLAGALSCV |
| | FLPQPHGESL |
| 801 | QIYPFITALA IRGNLAAFQE SGDHAREFSL HRPLTDVSLP |
| | VGIRASWKNH |
| 851 | HRVPLVWLTE ISYRSTLYRQ DPELHSKLLI SQGTWTTQAT |
| | PVTYNALGIK |
| 901 | VKNTMQVFPK VTLSLDYSAD ISSSTLSHYL NVASRMRF* |

A predicted signal peptide is highlighted.

The cp6751 nucleotide sequence <SEQ ID 6> is:

| | |
|---|---|
| 1 | ATGCGCTTTT TTTGCTTCGG AATGTTGCTT CCTTTTACTT |
| | TTGTATTGGC |
| 51 | TAATGAAGGT CTCCAACTTC CTTTGGAGAC CTATATTACA |
| | TTAAGTCCTG |
| 101 | AATATCAAGC AGCCCCTCAA GTAGGGTTTA CTCATAACCA |
| | AAATCAAGAT |
| 151 | CTCGCAATTG TCGGGAATCA CAATGATTTC ATCTTGGACT |
| | ATAAGTACTA |
| 201 | TCGGTCGAAT GGAGGTGCTC TTACCTGTAA GAATCTTCTG |
| | ATCTCTGAAA |
| 251 | ATATAGGGAA TGTCTTCTTT GAGAAGAATG TCTGTCCCAA |
| | TTCTGGCGGG |
| 301 | GCAATTTATG CTGCTCAAAA TTGCACGATC TCCAAGAATC |
| | AGAACTATGC |
| 351 | ATTTACTACA AACTTGGTCT CTGACAATCC TACAGCCACT |
| | GCGGGATCAC |
| 401 | TATTGGGTGG AGCTCTCTTT GCCATAAATT GCTCTATTAC |
| | TAATAACCTA |
| 451 | GGACAGGGAA CTTTCGTTGA CAATCTCGCT TTAAATAAGG |
| | GGGGTGCCCT |
| 501 | CTATACTGAG ACGAACTTAT CTATTAAAGA CAATAAAGGC |
| | CCGATCATAA |
| 551 | TCAAGCAGAA TCGGGCACTA AATTCGGACA GTTTAGGAGG |
| | AGGGATTTAT |
| 601 | AGTGGGAACT CTCTAAATAT AGAGGGAAAT TCTGGAGCTA |
| | TACAGATCAC |
| 651 | AAGCAACTCT TCAGGATCTG GGGGAGGCAT ATTTTCTACC |
| | CAAACACTCA |
| 701 | CGATCTCCTC GAATAAAAAA CTCATAGAAA TCAGTGAAAA |
| | TTCCGCGTTC |
| 751 | GCAAATAACT ATGGATCGAA CTTCAATCCA GGAGGAGGAG |
| | GTCTTACTAC |
| 801 | CACCTTTTGC ACGATATTGA ACAACCGAGA AGGGGTACTC |
| | TTTAACAATA |
| 851 | ACCAAAGCCA GAGCAACGGT GGAGCCATTC ATGCGAAATC |
| | TATCATTATC |
| 901 | AAAGAAAATG GTCCTGTATA CTTTTTAAAT AACACTGCAA |
| | CTCGGGGAGG |
| 951 | GGCTCTCCTC AACTTACCAG CAGGTTCTGG AAACGGAAGC |
| | TTCATCTTAT |
| 1001 | CTGCAGATAA TGGAGATATT ATCTTTAACA ATAATACGGC |
| | CTCCAAGCAT |
| 1051 | GCCCTCAATC CTCCATACAG AAACGCCATT CACTCGACTC |
| | CTAATATGAA |
| 1101 | TCTGCAAATA GGAGCCCGTC CCGGCTATCG AGTGCTGTTC |
| | TATGATCCCA |
| 1151 | TAGAACATGA GCTCCCTTCC TCCTTCCCCA TACTCTTTAA |
| | TTTCGAAACC |
| 1201 | GGTCATACAG GTACAGTTTT ATTTTCAGGG GAACATGTAC |
| | ACCAGAACTT |
| 1251 | TACCGATGAA ATGAATTTCT TTTCCTATTT AAGGAACACT |
| | TCGGAACTAC |
| 1301 | GTCAAGGAGT CCTTGCTGTT GAAGATGGTG CGGGGCTGGC |
| | CTGCTATAAG |
| 1351 | TTCTTCCAAC GAGGAGGCAC TCTACTTCTA GGTCAAGGTG |
| | CGGTGATCAC |
| 1401 | GACAGCAGGA ACGATTCCCA CACCATCCTC AACACCAACG |
| | ACAGTAGGAA |
| 1451 | GTACTATAAC TTTAAATCAC ATTGCCATTG ACCTTCCTTC |
| | TATTCTTTCT |
| 1501 | TTTCAAGCTC AGGCTCCAAA AATTTGGATT TACCCCACAA |
| | AAACAGGATC |
| 1551 | TACCTATACT GAAGATTCCA ACCCGACAAT CACAATCTCA |
| | GGAACTCTCA |
| 1601 | CCTTACGCAA CAGCAACAAC GAAGATCCCT ACGATAGTCT |
| | GGATCTCTCG |
| 1651 | CACTCTCTTG AGAAAGTTCC CCTTCTTTAT ATTGTCGATG |
| | TCGCTGCACA |
| 1701 | AAAAATTAAC TCTTCGCAAC TGGATCTATC CACATTAAAT |
| | TCTGGCGAAC |
| 1751 | ACTATGGGTA TCAAGGCATC TGGTCGACCT ATTGGGTAGA |
| | AACTACAACA |
| 1801 | ATCACGAACC CTACATCTCT ACTAGGCGCG AATACAAAAC |
| | ACAAGCTGCT |
| 1851 | CTATGCAAAC TGGTCTCCTC TAGGCTACCG TCCTCATCCC |
| | GAACGTCGAG |
| 1901 | GAGAATTCAT TACGAATGCC TTGTGGCAAT CGGCATATAC |
| | GGCTCTTGCA |
| 1951 | GGACTCCACT CCCTCTCCTC CTGGGATGAA GAGAAGGGTC |
| | ATGCAGCTTC |
| 2001 | CCTACAAGGC ATTGGTCTTC TGGTTCATCA AAAAGACAAA |
| | AACGGTTTTA |
| 2051 | AGGGATTTCG TAGTCATATG ACAGGTTATA GTGCTACCAC |
| | CGAAGCAACC |
| 2101 | TCTTCTCAAA GTCCGAATTT CTCTTTAGGA TTTGCTCAGT |
| | TCTTCTCCAA |
| 2151 | AGCTAAAGAA CATGAATCTC AAAATAGCAC GTCCTCTCAC |
| | CACTATTTCT |
| 2201 | CTGGAATGTG CATAGAAAAT ACTCTCTTCA AAGAGTGGAT |
| | ACGTCTATCT |
| 2251 | GTGTCTCTTG CTTATATGTT TACCTCGGAA CTACCCCATA |
| | CAATGTATCA |
| 2301 | GGGTCTCCTG GAAGGGAACT CTCAGGGATC TTTCCACAAC |
| | CATACCTTAG |
| 2351 | CAGGGGCTCT CTCCTGTGTT TTCTTACCTC AACCTCACGG |
| | CGAGTCCCTG |
| 2401 | CAGATCTATC CCTTTATTAC TGCCTTAGCC ATCCGAGGAA |
| | ATCTTGCTGC |
| 2451 | GTTTCAAGAA TCTGGAGACC ATGCTCGGGA ATTTTCCCTA |
| | CACCGCCCCC |
| 2501 | TAACGGACGT CTCCCTCCCT GTAGGAATCC GCGCTTCTTG |
| | GAAGAACCAC |
| 2551 | CACCGAGTTC CCCTAGTCTG GCTCACAGAA ATTTCCTATC |
| | GCTCTACTCT |
| 2601 | CTATAGGCAA GATCCTGAAC TCCACTCGAA ATTACTGATT |
| | AGCCAAGGTA |
| 2651 | CGTGGACGAC GCAGGCCACT CCTGTGACCT ACAATGCTTT |
| | AGGGATCAAA |
| 2701 | GTGAAAAATA CCATGCAGGT GTTTCCTAAA GTCACTCTCT |
| | CCTTAGATTA |
| 2751 | CTCTGCGGAT ATTTCTTCCT CCACGCTGAG TCACTACTTA |
| | AACGTGGCGA |
| 2801 | GTAGAATGAG ATTTTAA |

The PSORT algorithm predicts an outer membrane location (0.923).

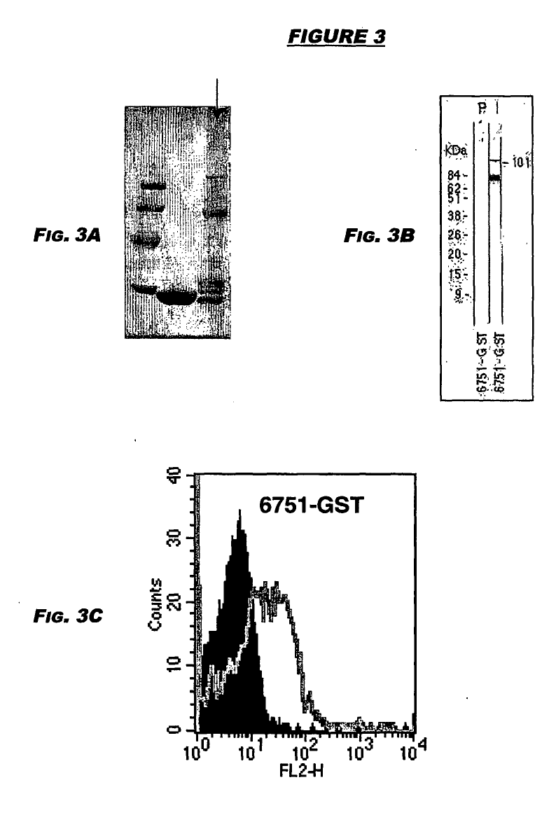

The protein was expressed in E. coli and purified as a GST-fusion product, as shown in FIG. 3A, and also in his-tagged form. The GST-fusion recombinant protein was used to immunise mice, whose sera were used in a Western blot (FIG. 3B) and for FACS analysis (FIG. 3C).

This protein also showed good cross-reactivity with human sera, including sera from patients with pneumonitis.

These experiments show that cp6751 is a surface-exposed and immunoaccessible protein, and that it is a useful immunogen. These properties are not evident from the sequence alone.

### Example 4

The following C. pneumoniae protein (PID 4376752) was expressed <SEQ ID 7; cp6752>:

| | |
|---|---|
| 1 | MFGMTPAVYS LQTDSLEKFA LERDEEFRTS FPLLDSLSTL |
| | TGFSPITTFV |
| 51 | GNRHNSSQDI VLSNYKSIDN ILLLWTSAGG AVSCNNFLLS |
| | NVEDHAFFSK |
| 101 | NLAIGTGGAI ACQGACTITK NRGPLIFFSN RGLNNASTGG |
| | ETRGGAIACN |
| 151 | GDFTISQNQG TFYFVNNSVN NWGGALSTNG HCRIQSNRAP |
| | LLFFNNTAPS |
| 201 | GGGALRSENT TISDNTRPIY FKNNCGNNGG AIQTSVTVAI |
| | KNNSGSVIFN |
| 251 | NNTALSGSIN SGNGSGGAIY TTNLSIDDNP GTILFNNKYC |
| | IRDGGAICTQ |
| 301 | FLTIKNSGHV YFTNNQGNWG GALMLLQDST CLLFAEQGNI |
| | AFQNNEVFLT |
| 351 | TFGRYNAIHC TPNSNLQLGA NKGYTTAFFD PIEHQHPTTN |
| | PLIFNPNANH |
| 401 | QGTILFSSAY IPEASDYENN FISSSKNTSE LRNGVLSIED |
| | RAGWQFYKFT |
| 451 | QKGGILKIGH AASIATTANS ETPSTSVGSQ VIINNLAINL |
| | PSILAKGKAP |
| 501 | TLWIRPLQSS APFTEDNNPT ITLSGPLTLL NEENRDPYDS |
| | IDLSEPLQNI |
| 551 | HLLSLSDVTA RHINTDNFHP ESLNATEHYG YQGIWSPYWV |
| | ETITTTNNAS |
| 601 | IETANTLYRA IYANWTPLGY KVNPEYQGDL ATTPLWQSFH |
| | TMFSLLRSYN |
| 651 | RTGDSDIERP FLEIQGIADG LFVHQNSIPG APGFRIQSTG |
| | YSLQASSETS |
| 701 | HLQKISLGFA QFFTRTKEIG SSNNVSAHNT VSSLYVELPW |
| | FQEAFATSTV |
| 751 | LAYGYGDHHL HSLHPSHQEQ AEGTCYSHTL AAAIGCSFPW |
| | QQKSYLHLSP |
| 801 | FVQAIAIRSH QTAFEEIGDN PRKFVSQKPF YNLTLPLGIQ |
| | GKWQSKFHVP |
| 851 | TEWTLELSYQ PVLYQQNPQI GVTLLASGGS WDILGHNYVR |
| | NALGYKVHNQ |
| 901 | TALFRSLDLF IDYQGSVSSS TSTHHLQAGS TLKF* |

The cp6752 nucleotide sequence <SEQ ID 8> is:

| | |
|---|---|
| 1 | ATGTTCGGGA TGACTCCTGC AGTGTATAGT TTACAAACGG |
| | ACTCCCTTGA |
| 51 | AAAGTTTGCT TTAGAGAGGG ATGAAGAGTT TCGTACGAGC |
| | TTTCCTCTCT |
| 101 | TAGACTCTCT CTCCACTCTT ACAGGATTTT CTCCAATAAC |
| | TACGTTTGTT |
| 151 | GGAAATAGAC ATAATTCCTC TCAAGACATT GTACTTTCTA |
| | ACTACAAGTC |
| 201 | TATTGATAAC ATCCTTCTTC TTTGGACATC GGCTGGGGGA |
| | GCTGTGTCCT |
| 251 | GTAATAATTT CTTATTATCA AATGTTGAAG ACCATGCCTT |
| | CTTCAGTAAA |
| 301 | AATCTCGCGA TTGGGACTGG AGGCGCGATT GCTTGCCAGG |
| | GAGCCTGCAC |
| 351 | AATCACGAAG AATAGAGGAC CCCTTATTTT TTTCAGCAAT |
| | CGAGGTCTTA |
| 401 | ACAATGCGAG TACAGGAGGA GAAACTCGTG GGGGTGCGAT |
| | TGCCTGTAAT |
| 451 | GGAGACTTCA CGATTTCTCA AAATCAAGGG ACTTTCTACT |
| | TTGTCAACAA |
| 501 | TTCCGTCAAC AACTGGGGAG GAGCCCTCTC CACCAATGGA |
| | CACTGCCGCA |
| 551 | TCCAAAGCAA CAGGGCACCT CTACTCTTTT TTAACAATAC |
| | AGCCCCTAGT |
| 601 | GGAGGGGGTG CGCTTCGTAG TGAAAATACA ACGATCTCTG |
| | ATAACACGCG |
| 651 | TCCTATTTAT TTTAAGAACA ACTGTGGGAA CAATGGCGGG |
| | GCCATTCAAA |
| 701 | CAAGCGTTAC TGTTGCGATA AAAAATAACT CCGGGTCGGT |
| | GATTTTCAAT |
| 751 | AACAACACAG CGTTATCTGG TTCGATAAAT TCAGGAAATG |
| | GTTCAGGAGG |
| 801 | GGCGATTTAT ACAACAAACC TATCCATAGA CGATAACCCT |
| | GGAACTATTC |
| 851 | TTTTCAATAA TAACTACTGC ATTCGCGATG GCGGAGCTAT |
| | CTGTACACAA |
| 901 | TTTTTGACAA TCAAAAATAG TGGCCACGTA TATTTCACCA |
| | ACAATCAAGG |
| 951 | AAACTGGGGA GGTGCTCTTA TGCTCCTACA GGACAGCACC |
| | TGCCTACTCT |
| 1001 | TCGCGGAACA AGGAAATATC GCATTTCAAA ATAATGAGGT |
| | TTTCCTCACC |
| 1051 | ACATTTGGTA GATACAACGC CATACATTGT ACACCAAATA |
| | GCAACTTACA |
| 1101 | ACTTGGAGCT AATAAGGGGT ATACGACTGC TTTTTTTGAT |
| | CCTATAGAAC |
| 1151 | ACCAACATCC AACTACAAAT CCTCTAATCT TTAATCCCAA |
| | TGCGAACCAT |
| 1201 | CAGGGAAGCA TCTTATTTTC TTCAGCCTAT ATCCCAGAAG |
| | CTTCTGACTA |
| 1251 | CGAAAATAAT TTCATTAGCA GCTCGAAAAA TACCTCTGAA |
| | CTTCGCAATG |
| 1301 | GTGTCCTCTC TATCGAGGAT CGTGCGGGAT GGCAATTCTA |
| | TAAGTTCACT |
| 1351 | CAAAAAGGAG GTATCCCTAA ATTAGGGCAT GCGGCGAGTA |
| | TTGCAACAAC |
| 1401 | TGCCAACTCT GAGACTCCAT CAACTAGTGT AGGCTCCCAG |
| | GTCATCATTA |
| 1451 | ATAACCTTGC GATTAACCTC CCCTCGATCT TAGCAAAAGG |
| | AAAAGCTCCT |
| 1501 | ACCTTGTGGA TCCGTCCTCT ACAATCTAGT GCTCCTTTCA |
| | CAGAGGACAA |
| 1551 | TAACCCTACA ATTACTTTAT CAGGTCCTCT GACACTCTTA |
| | AATGAGGAAA |
| 1601 | ACCGCGATCC CTACGACAGT ATAGATCTCT CTGAGCCTTT |
| | ACAAAACATT |
| 1651 | CATCTTCTTT CTTTATCGGA TGTAACAGCA CGTCATATCA |
| | ATACCGATAA |
| 1701 | CTTTCATCCT GAAAGCTTAA ATGCGACTGA GCATTACGGT |
| | TATCAAGGCA |
| 1751 | TCTGGTCTCC TTATTGGGTA GAGACGATAA CAACAACAAA |
| | TAACGCTTCT |
| 1801 | ATAGAGACGG CAAACACCCT CTACAGAGCT CTGTATGCCA |
| | ATTGGACTCC |
| 1851 | CTTAGGATAT AAGGTCAATC CTGAATACCA AGGAGATCTT |
| | GCTACGACTC |
| 1901 | CCCTATGGCA ATCCTTTCAT ACTATGTTCT CTCTATTAAG |
| | AAGTTATAAT |
| 1951 | CGAACTGGTG ATTCTGATAT CGAGAGGCCT TTCTTAGAAA |
| | TTCAAGGGAT |
| 2001 | TGCCGACGGC CTCTTTGTTC ATCAAAATAG CATCCCCGGG |
| | GCTCCAGGAT |
| 2051 | TCCGTATCCA ATCTACAGGG TATTCCTTAC AAGCATCCTC |
| | CGAAACTTCT |
| 2101 | TTACATCAGA AAATCTCCTT AGGTTTTGCA CAGTTCTTCA |
| | CCCGCACTAA |
| 2151 | AGAAATCGGA TCAAGCAACA ACGTCTCGGC TCACAATACA |
| | GTCTCTTCAC |
| 2201 | TTTATGTTGA GCTTCCGTGG TTCCAAGAGG CCTTTGCAAC |
| | ATCCACAGTG |
| 2251 | TTAGCGTATG GCTATGGGGA CCATCACCTC CACAGCCTAC |
| | ATCCCTCACA |
| 2301 | TCAAGAACAG GCAGAAGGGA CGTGTTATAG CCATACATTA |
| | GCAGCAGCTA |
| 2351 | TCGGCTGTTC TTTCCCTTGG CAACAGAAAT CCTATCTTCA |
| | CCTCAGCCCG |
| 2401 | TTCGTTCAGG CAATTGCAAT ACGTTCTCAC CAAACAGCGT |
| | TCGAAGAGAT |
| 2451 | TGGTGACAAT CCCCGAAAGT TTGTCTCTCA AAAGCCTTTC |
| | TATAATCTGA |
| 2501 | CCTTACCTCT AGGAATCCAA GGAAAATGGC AGTCAAAATT |
| | CCACGTACCT |
| 2551 | ACAGAATGGA CTCTAGAACT TTCTTACCAA CCGGTACTCT |
| | ATCAACAAAA |
| 2601 | TCCCCAAATC GGTCTCACGC TACTTGCGAG CGGAGGTTCC |
| | TGGGATATCC |
| 2651 | TAGGCCATAA CTATGTTCGC AATGCTTTAG GGTACAAAGT |
| | CCACAATCAA |
| 2701 | ACTGCGCTCT TCCGTTCTCT CGATCTATTC TTGGATTACC |
| | AAGGATCGGT |
| 2751 | CTCCTCCTCG ACATCTACGC ACCATCTCCA AGCAGGAAGT |
| | ACCTTAAAAT |
| 2801 | TCTAA |

The PSORT algorithm predicts a cytoplasmic location (0.138).

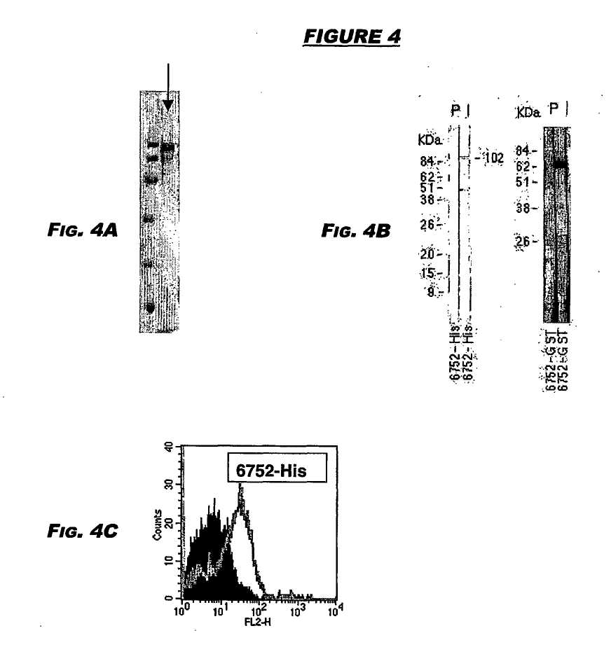

The protein was expressed in E. coli and purified as a his-tag product, as shown in FIG. 4A, and also as a GST-fusion. The recombinant proteins were used to immunise mice, whose sera were used in a Western blot (4B) and the his-tagged protein was used for FACS analysis (4C).

The cp6752 protein was also identified in the 2D-PAGE experiment (Cpn0467).

These experiments show that cp6752 is a surface-exposed and immunoaccessible protein, and that it is a useful immunogen. These properties are not evident from the sequence alone.

### Example 5

The following C. pneumoniae protein (PID 4376850) was expressed <SEQ ID 9; cp6850>:

| | |
|---|---|
| 1 | MKKAVLIAAM FCGVVSLSSC CRIVDCCFED PCAPSSCNPC |
| | EVIRKKERSC |
| 51 | GGNACGSYVP SCSNPCGSTE CNSQSPQVKG CTSPDGRCKQ * |

A predicted signal peptide is highlighted.

The cp6850 nucleotide sequence <SEQ ID 10> is:

| | |
|---|---|
| 1 | ATGAAGAAAG CTGTTTTAAT TGCTGCAATG TTTTGTGGAG |
| | TAGTTAGCTT |
| 51 | AAGTAGCTGC TGCCGCATTG TAGATTGTTG TTTTGAGGAT |
| | CCTTGCGCAC |
| 101 | CCTCTTCTTG CAATCCTTGT GAAGTAATAA GAAAAAAAGA |
| | AAGATCTTGC |
| 151 | GGCGGTAATG CTTGTGGGTC CTACGTTCCT TCTTGTTCTA |
| | ATCCATGTGG |
| 201 | TTCAACAGAG TGTAACTCTC AAAGCCCACA AGTTAAAGGT |
| | TGTACATCAC |
| 251 | CTGATGGCAG ATGCAAACAG TAA |

The PSORT algorithm predicts an inner membrane location (0.329).

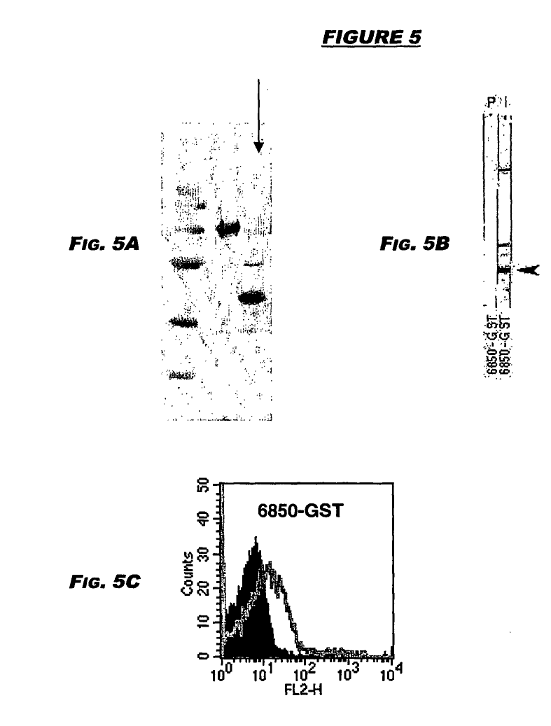

The protein was expressed in E. coli and purified as a GST-fusion product, as shown in FIG. 5A. The recombinant protein was used to immunise mice, whose sera were used in a Western blot (FIG. 5B) and for FACS analysis (FIG. 5B). A his-tagged protein was also expressed.

These experiments show that cp6850 is a surface-exposed and immunoaccessible protein, and that it is a useful immunogen. These properties are not evident from the sequence alone.

### Example 6

The following C. pneumoniae protein (PID 4376900) was expressed <SEQ ID 11; cp6900>:

| | |
|---|---|
| 1 | MKIKFSWKVN FLICLLAVGL IFFGCSRVKR EVLVGRDATW |
| | FPKQFGIYTS |
| 51 | DTNAFLNDLV SEINYKENLN INIVNQDWVH LFENLDDKKT |
| | QGAFTSVLPT |
| 101 | LEMLEHYQFS DPILLTGPVL VVAQDSPYQS IEDLKGRLIG |
| | VYKFDSSVLV |
| 151 | AQNIPDAVIS LYQHVPIALE ALTSNCYDAL LAPVIEVTAL |
| | IETAYKGRLK |
| 201 | IISKPLNADG LRLAILKGTN GDLLEGFNAG LVKTRRSGKY |
| | DAIKQRYRLP |

The cp6900 nucleotide sequence <SEQ ID 12> is:

| | |
|---|---|
| 1 | GTGAAGATAA AATTTTCTTG GAAGGTAAAT TTTTTAATAT |
| | GTTTACTGGC |
| 51 | TGTGGGACTG ATCTTTTTCG GGTGCTCTCG AGTAAAAAGA |
| | GAAGTTCTCG |
| 101 | TAGGTCGTGA TGCCACCTGG TTTCCAAAAC AATTCGGCAT |
| | TTATACATCC |
| 151 | GATACCAACG CATTTTTAAA CGATCTTGTT TCTGAGATTA |
| | ACTATAAAGA |
| 201 | GAATCTAAAT ATTAATATTG TAAATCAAGA TTGGGTGCAT |
| | CTCTTTGAGA |
| 251 | ATTTAGATGA TAAAAAGACC CAAGGAGCAT TTACATCTGT |
| | ATTGCCTACT |
| 301 | CTTGAGATGC TCGAACACTA TCAATTTTCT GATCCCATTT |
| | TACTCACAGG |
| 351 | TCCTGTCCTT GTCGTCGCTC AAGACTCTCC TTACCAATCT |
| | ATAGAGGATC |
| 401 | TTAAAGGTCG TCTTATTGGA GTGTATAAGT TTGACTCTTC |
| | AGTTCTTGTA |
| 451 | GCTCAAAATA TCCCTGACGC TGTGATTAGC CTCTACCAAC |
| | ATGTTCCAAT |
| 501 | AGCATTGGAA GCCTTAACAT CGAATTGTTA CGACGCTCTT |
| | CTAGCTCCTG |
| 551 | TAATTGAAGT GACCGCGCTA ATAGAAACAG CATATAAAGG |
| | AAGACTGAAA |
| 601 | ATTATTTCAA AACCCTTAAA CGCAGATGGT TTGCGGCTTG |
| | CAATACTGAA |
| 651 | AGGGACAAAC GGAGATTTGC TTGAAGGGTT TAACGCAGGA |
| | CTTGTGAAAA |
| 701 | CACGACGCTC AGGAAAATAC GATGCTATAA AACAGCGGTA |
| | TCGTCTTCCC |
| 751 | TAA |

The PSORT algorithm predicts an inner membrane location (0.452).

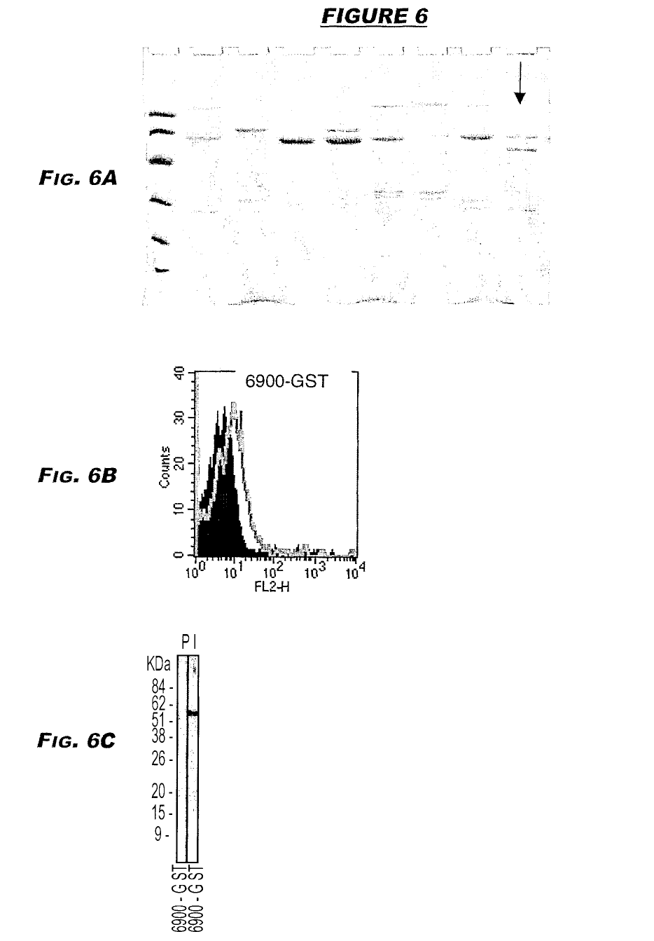

The protein was expressed in E. coli and purified as a GST-fusion product, as shown in FIG. 6A. The recombinant protein was used to immunise mice, whose sera were used for FACS analysis (FIG. 6B). A his-tagged protein was also expressed.

The cp6900 protein was also identified in the 2D-PAGE experiment (Cpn0604).

These experiments show that cp6900 is a surface-exposed and immunoaccessible protein, and that it is a useful immunogen. These properties are not evident from the sequence alone.

### Example 7

The following C. pneumoniae protein (PID 4377033) was expressed <SEQ ID 13; cp7033>:

| | |
|---|---|
| 1 | MVNPIGPGPI DETERTPPAD LSAQGLEASA ANKSAEAQRI |
| | AGAEAKPKES |
| 51 | KTDSVERWSI LRSAVNALMS LADKLGIASS NSSSSTSRSA |
| | DVDSTTATAP |
| 101 | TPPPPTFDDY KTQAQTAYDT IFTSTSLADI QAALVSLQDA |
| | VTNIKDTAAT |
| 151 | DEETAIAAEW ETKNADAVKV GAQITELAKY ASDNQAILDS |
| | LGKLTSFDLL |
| 201 | QAALLQSVAN NNKAAELLKE MQDNPVVPGK TPAIAQSLVD |
| | QTDATATQIE |
| 251 | KDGNAIRDAY FAGQNASGAV ENAKSNNSIS NIDSAKAAIA |
| | TAKTQIAEAQ |
| 301 | KKFPDSPILQ EAEQMVIQAE KDLKNIKPAD GSDVPNPGTT |
| | VGGSKQQGSS |
| 351 | IGSIRVSMLL DDAENETASI LMSGFRQMIH MFNTENPDSQ |
| | AAQQELAAQA |
| 401 | RAAKAAGDDS AAAALADAQK ALEAALGKAG QQQGILNALG |
| | QIASAAVVSA |
| 451 | GVPPAAASSI GSSVKQLYKT SKSTGSDYKT QISAGYDAYK |
| | SINDAYGRAR |
| 501 | NDATRDVINN VSTPALTRSV PRARTEARGP EKTDQALARV |
| | ISGNSRTLGD |
| 551 | VYSQVSALQS VMQIIQSNPQ ANNEEIRQKL TSAVTKPPQF |
| | GYPYVQLSND |
| 601 | STQKFIAKLE SLFAEGSRTA AEIKALSFET NSLFIQQVLV |
| | NIGSLYSGYL |
| 651 | Q* |

The cp7033 nucleotide sequence <SEQ ID 14> is:

| | |
|---|---|
| 1 | ATGGTTAATC CTATTGGTCC AGGTCCTATA GACGAAACAG |
| | AACGCACACC |
| 51 | TCCCGCAGAT CTTTCTGCTC AAGGATTGGA GGCGAGTGCA |
| | GCAAATAAGA |
| 101 | GTGCGGAAGC TCAAAGAATA GCAGGTGCGG AAGCTAAGCC |
| | TAAAGAATCT |
| 151 | AAGACCGATT CTGTAGAGCG ATGGAGCATC TTGCGTTCTG |
| | CAGTGAATGC |
| 201 | TCTCATGAGT CTGGCAGATA AGCTGGGTAT TGCTTCTAGT |
| | AACAGCTCGT |
| 251 | CTTCTACTAG CAGATCTGCA GACGTGGACT CAACGACAGC |
| | GACCGCACCT |
| 301 | ACGCCTCCTC CACCCACGTT TGATGATTAT AAGACTCAAG |
| | CGCAAACAGC |
| 351 | TTACGATACT ATCTTTACCT CAACATCACT AGCTGACATA |
| | CAGGCTGCTT |
| 401 | TGGTGAGCCT CCAGGATGCT GTCACTAATA TAAAGGATAC |
| | AGCGGCTACT |
| 451 | GATGAGGAAA CCGCAATCGC TGCGGAGTGG GAAACTAAGA |
| | ATGCCGATGC |
| 501 | AGTTAAAGTT GGCGCGCAAA TTACAGAATT AGCGAAATAT |
| | GCTTCGGATA |
| 551 | ACCAAGCGAT TCTTGACTCT TTAGGTAAAC TGACTTCCTT |
| | CGACCTCTTA |
| 601 | CAGGCTGCTC TTCTCCAATC TGTAGCAAAC AATAACAAAG |
| | CAGCTGAGCT |
| 651 | TCTTAAAGAG ATGCAAGATA ACCCAGTAGT CCCAGGGAAA |
| | ACGCCTGCAA |
| 701 | TTGCTCAATC TTTAGTTGAT CAGACAGATG CTACAGCGAC |
| | ACAGATAGAG |
| 751 | AAAGATGGAA ATGCGATTAG GGATGCATAT TTTGCAGGAC |
| | AGAACGCTAG |
| 801 | TGGAGCTGTA GAAAATGCTA AATCTAATAA CAGTATAAGC |
| | AACATAGATT |
| 851 | CAGCTAAAGC AGCAATCGCT ACTGCTAAGA CACAAATAGC |
| | TGAAGCTCAG |
| 901 | AAAAAGTTCC CCGACTCTCC AATTCTTCAA GAAGCGGAAC |
| | AAATGGTAAT |
| 951 | ACAGGCTGAG AAAGATCTTA AAAATATCAA ACCTGCAGAT |
| | GGTTCTGATG |
| 1001 | TTCCAAATCC AGGAACTACA GTTGGAGGCT CCAAGCAACA |
| | AGGAAGTAGT |
| 1051 | ATTGGTAGTA TTCGTGTTTC CATGCTGTTA GATGATGCTG |
| | AAAATGAGAC |
| 1101 | CGCTTCCATT TTGATGTCTG GGTTTCGTCA GATGATTCAC |
| | ATGTTCAATA |
| 1151 | CGGAAAATCC TGATTCTCAA GCTGCCCAAC AGGAGCTCGC |
| | AGCACAAGCT |
| 1201 | AGAGCAGCGA AAGCCGCTGG AGATGACAGT GCTGCTGCAG |
| | CGCTGGCAGA |
| 1251 | TGCTCAGAAA GCTTTAGAAG CGGCTCTAGG TAAAGCTGGG |
| | CAACAACAGG |
| 1301 | GCATACTCAA TGCTTTAGGA CAGATCGCTT CTGCTGCTGT |
| | TGTGAGCGCA |
| 1351 | GGAGTTCCTC CCGCTGCAGC AAGTTCTATA GGGTCATCTG |
| | TAAAACAGCT |
| 1401 | TTACAAGACC TCAAAATCTA CAGGTTCTGA TTATAAAACA |
| | CAGATATCAG |
| 1451 | CAGGTTATGA TGCTTACAAA TCCATCAATG ATGCCTATGG |
| | TAGGGCACGA |
| 1501 | AATGATGCGA CTCGTGATGT GATAAACAAT GTAAGTACCC |
| | CCGCTCTCAC |
| 1551 | ACGATCCGTT CCTAGAGCAC GAACAGAAGC TCGAGGACCA |
| | GAAAAAACAG |
| 1601 | ATCAAGCCCT CGCTAGGGTG ATTTCTGGCA ATAGCAGAAC |
| | TCTTGGAGAT |
| 1651 | GTCTATAGTC AAGTTTCGGC ACTACAATCT GTAATGCAGA |
| | TCATCCAGTC |
| 1701 | GAATCCTCAA GCGAATAATG AGGAGATCAG ACAAAAGCTT |
| | ACATCGGCAG |
| 1751 | TGACAAAGCC TCCACAGTTT GGCTATCCTT ATGTGCAACT |
| | TTCTAATGAC |
| 1801 | TCTACACAGA AGTTCATAGC TAAATTAGAA AGTTTGTTTG |
| | CTGAAGGATC |
| 1851 | TAGGACAGCA GCTGAAATAA AAGCACTTTC CTTTGAAACG |
| | AACTCCTTGT |
| 1901 | TTATTCAGCA GGTGCTGGTC AATATCGGCT CTCTATATTC |
| | TGGTTATCTC |
| 1951 | CAATAA |

The PSORT algorithm predicts a cytoplasmic location (0.272).

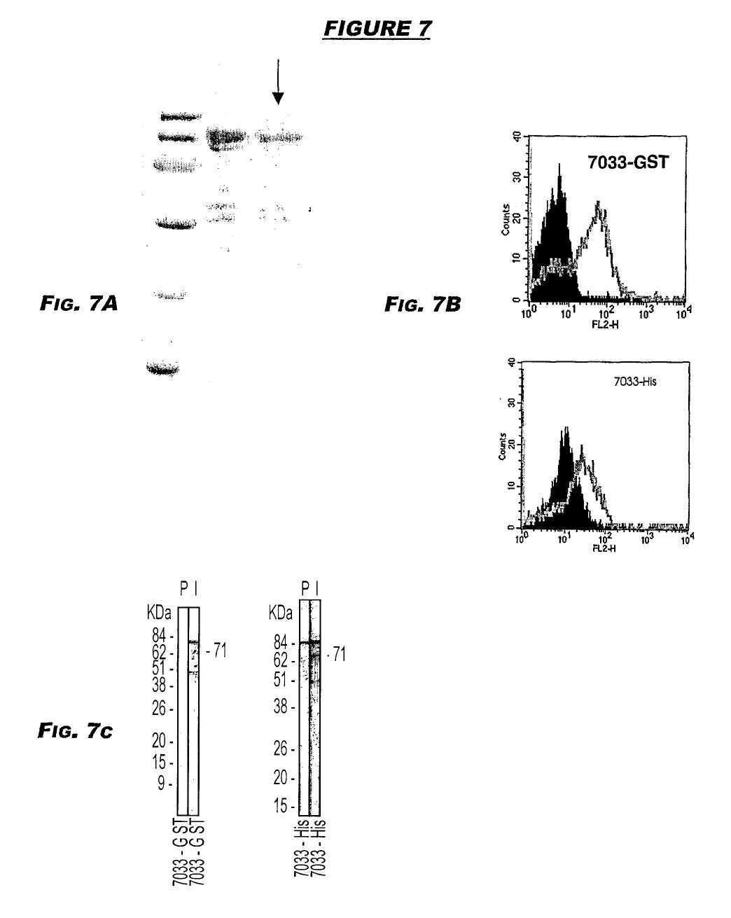

The protein was expressed in E. coli and purified as a GST-fusion product, as shown in FIG. 7A. A his-tagged protein was also expressed. The recombinant proteins were used to immunise mice, whose sera were used for FACS (FIG. 7B) and Western blot (7C) analyses.

The cp7033 protein was also identified in the 2D-PAGE experiment (Cpn0728) and showed good cross-reactivity with human sera, including sera from patients with pneumonitis.

These experiments show that cp7033 a surface-exposed and immunoaccessible protein, and that it is a useful immunogen. These properties are not evident from the sequence alone.

### Example 8

The following C. pneumoniae protein (PID 6172321) was expressed <SEQ ID 15; cp0017>:

| | |
|---|---|
| 1 | MGIKGTGIIV WVDDATAKTK NATLTWTKTG YKPNPERQGP |
| | LVPNSLWGSF |
| 51 | VDVRSIQSLM DRSTSSLSSS TNLWVSGIAD FLHEDQKGNQ |
| | RSYRHSSAGY |
| 101 | ALGGGFFTAS ENFFNFAFCQ LFGYDKDHLV AKNHTHVYAG |
| | AMSYRHLGES |
| 151 | KTLAKILSGN SDSLPFVFNA RFAYGHTDNN MTTKYTGYSP |
| | VKGSWGNDAF |
| 201 | GIECGGAIPV VASGRRSWVD THTPFLNLEM IYAHQNDFKE |
| | NGTEGRSFQS |
| 251 | EDLFNLAVPV GIKFEKFSDK STYDLSIAYV PDVIRNDPGC |
| | TTTLMVSGDS |
| 301 | WSTCGTSLSR QALLVRAGNH HAFASNFEVF SQFEVELRGS |
| | SRSYAIDLGG |
| 351 | RFGF* |

The cp0017 nucleotide sequence <SEQ ID 16> is:

| | |
|---|---|
| 1 | ATGGGTATCA AGGGAACTGG AATAATTGTT TGGGTCGACG |
| | ATGCAACTGC |
| 51 | AAAAACAAAA AATGCTACCT TAACTTGGAC TAAAACAGGA |
| | TACAAGCCGA |
| 101 | ATCCAGAACG TCAGGGACCT TTGGTTCCTA ATAGCCTGTG |
| | GGGTTCTTTT |
| 151 | GTCGATGTCC GCTCCATTCA GAGCCTCATG GACCGGAGCA |
| | CAAGTTCGTT |
| 201 | ATCTTCGTCA ACAAATTTGT GGGTATCAGG AATCGCGGAC |
| | TTTTTGCATG |
| 251 | AAGATCAGAA AGGAAACCAA CGTAGTTATC GTCATTCTAG |
| | CGCGGGTTAT |
| 301 | GCATTAGGAG GAGGATTCTT CACGGCTTCT GAAAATTTCT |
| | TTAATTTTGC |
| 351 | TTTTTGTCAG CTTTTTGGCT ACGACAAGGA CCATCTTGTG |
| | GCTAAGAACC |
| 401 | ATACCCATGT ATATGCAGGG GCAATGAGTT ACCGACACCT |
| | CGGAGAGTCT |
| 451 | AAGACCCTCG CTAAGATTTT GTCAGGAAAT TCTGACTCCC |
| | TACCTTTTGT |
| 501 | CTTCAATGCT CGGTTTGCTT ATGGCCATAC CGACAATAAC |
| | ATGACCACAA |
| 551 | AGTACACTGG CTATTCTCCT GTTAAGGGAA GCTGGGGAAA |
| | TGATGCCTTC |
| 601 | GGTATAGAAT GTGGAGGAGC TATCCCGGTA GTTGCTTCAG |
| | GACGTCGGTC |
| 651 | TTGGGTGGAT ACCCACACGC CATTTCTAAA CCTAGAGATG |
| | ATCTATGCAC |
| 701 | ATCAGAATGA CTTTAAGGAA AACGGCACAG AAGGCCGTTC |
| | TTTCCAAAGT |
| 751 | GAAGACCTCT TCAATCTAGC GGTTCCTGTA GGGATAAAAT |
| | TTGAGAAATT |
| 801 | CTCCGATAAG TCTACGTATG ATCTCTCCAT AGCTTACGTT |
| | CCCGATGTGA |
| 851 | TTCGTAATGA TCCAGGCTGC ACGACAACTC TTATGGTTTC |
| | TGGGGATTCT |
| 901 | TGGTCGACAT GTGGTACAAG CTTGTCTAGA CAAGCTCTTC |
| | TTGTACGTGC |
| 951 | TGGAAATCAT CATGCCTTTG CTTCAAACTT TGAAGTTTTC |
| | AGTCAGTTTG |
| 1001 | AAGTCGAGTT GCGAGGTTCT TCTCGTAGCT ATGCTATCGA |
| | TCTTGGAGGA |
| 1051 | AGATTCGGAT TTTAA |

This sequence is frame-shifted with respect to cp0016.

The PSORT algorithm predicts a cytoplasmic location (0.075).

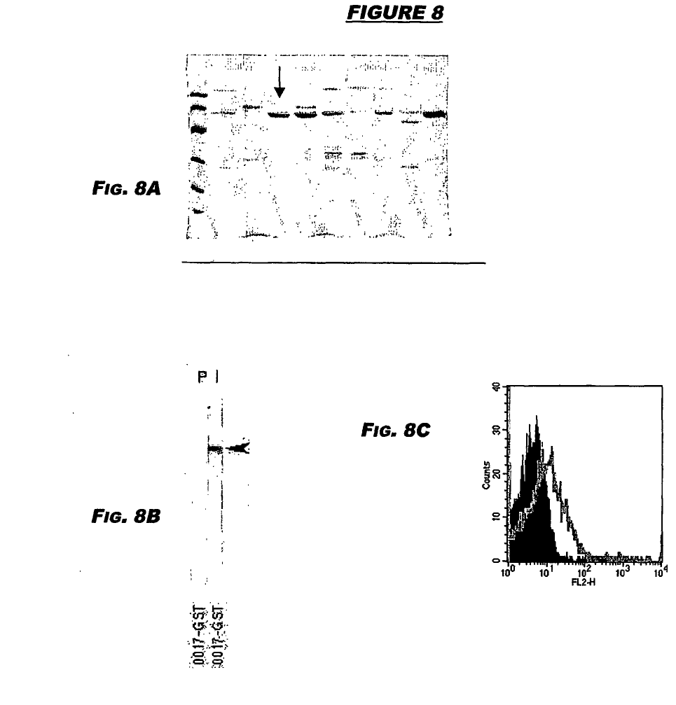

The protein was expressed in E. coli and purified as a GST-fusion product, as shown in FIG. 8A. The recombinant protein was used to immunise mice, whose sera were used in a Western blot (FIG. 8B) and for FACS analysis (FIG. 8C). A his-tagged protein was also expressed.

This protein also showed good cross-reactivity with human sera, including sera from patients with pneumonitis.

These experiments show that cp0017 is a surface-exposed and immunoaccessible protein, and that it is a useful immunogen. These properties are not evident from the sequence alone.

### Example 9

The following C. pneumoniae protein (PID 6172315) was expressed <SEQ ID 17; cp0014>:

| | |
|---|---|
| 1 | MKSSFPKFVF STFAIFPLSM IATETVLDSS ASFDGNKNGN |
| | FSVRESQEDA |
| 51 | GTTYLFKGNV TLENIPGTGT AITKSCFNNT KGDLTFTGNG |
| | NSLLFQTVDA |
| 101 | GTVAGAAVNS SVVDKSTTFI GFSSLSFIAS PGSSITTGKG |
| | AVSCSTGSLS |
| 151 | LTKMSVCSSA KTFQRIMAVL SPQKLFH* |

The cp0014 nucleotide sequence <SEQ ID 18> is:

| | |
|---|---|
| 1 | ATGAAGTCTT CTTTCCCCAA GTTTGTATTT TCTACATTTG |
| | CTATTTTCCC |
| 51 | TTTGTCTATG ATTGCTACCG AGACAGTTTT GGATTCAAGT |
| | GCGAGTTTCG |
| 101 | ATGGGAATAA AAATGGTAAT TTTTCAGTTC GTGAGAGTCA |
| | GGAAGATGCT |
| 151 | GGAACTACCT ACCTATTTAA GGGAAATGTC ACTCTAGAAA |
| | ATATTCCTGG |
| 201 | AACAGGCACA GCAATCACAA AAAGCTGTTT TAACAACACT |
| | AAGGGCGATT |
| 251 | TGACTTTCAC AGGTAACGGG AACTCTCTAT TGTTCCAAAC |
| | GGTGGATGCA |
| 301 | GGGACTGTAG CAGGGGCTGC TGTTAACAGC AGCGTGGTAG |
| | ATAAATCTAC |
| 351 | CACGTTTATA GGGTTTTCTT CGCTATCTTT TATTGCGTCT |
| | CCTGGAAGTT |
| 401 | CGATAACTAC CGGCAAAGGA GCCGTTAGCT GCTCTACGGG |
| | TAGCTTGAGT |
| 451 | TTGACAAAAA TCTCAGTTTG CTCTTCAGCA AAAACTTTTC |
| | AACGGATAAT |
| 501 | GGCGGTGCTA TCACCGCAAA AACTCTTTCA TTAA |

This protein is frame-shifted with respect to cp0015.

The PSORT algorithm predicts an inner membrane location (0.047).

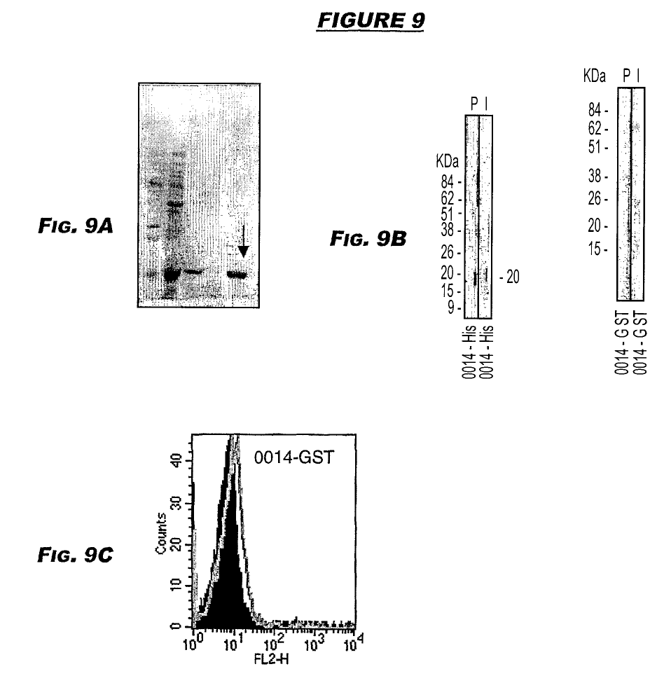

The protein was expressed in E. coli and purified as a his-tag product, as shown in FIG. 9A. A GST-fusion was also expressed. The recombinant proteins were used to immunise mice, whose sera were used in an immunoassay (FIG. 9B) and for FACS analysis (FIG. 9C).

This protein also showed good cross-reactivity with human sera, including sera from patients with pneumonitis.

These experiments suggest that cp0014 is a useful immunogen. These properties are not evident from the sequence alone.

### Example 10

The following C. pneumoniae protein (PID 6172317) was expressed <SEQ ID 19; cp0015>:

| | |
|---|---|
| 1 | MSALFSENTS SKKGGAIQTS DALTITGNQG EVSFSDNTSS |
| | DSGAAIFTEA |
| 51 | SVTISNNAKV SFIDNKVTGA SSSTTGDMSG GAICAYKTST |
| | DTKVTLTGNQ |
| 101 | MLLFSNNTST TAGGAIYVKK LELASGGLTL FSRNSVNGGT |
| | APKGGAIAIE |
| 151 | DSGELSLSAD SGDIVFLGNT VTSTTPGTNR SSIDLGTSAK |
| | MTALRSAAGR |
| 201 | AIYFYDPITT GSSTTVTDVL KVNETPADSA LQYTGNIIFT |
| | GEKLSETEAA |
| 251 | DSKNLTSKLL QPVTLSGGTL SLKHGVTLQT QAFTQQADSR |
| | LEMDVGTTLE |
| 301 | PADTSTINNL VINISSIDGA KKAKIETKAT SKNLTLSGTI |
| | TLLDPTGTFY |
| 351 | ENHSLRNPQS YDILELKASG TVTSTAVTPD PIMGEKFEYG |
| | YQGTWGPIVW |
| 401 | GTGASTTATF NWTKTGYIPN PERIGSLVPN SLWNAFIDIS |
| | SLHYLMETAN |
| 451 | EGLQGDRAFW CAGLSNFFHK DSTKTRRGFR HLSGGYVIGG |
| | NLHTCSDKIL |
| 501 | SAAFCQLFGR DRDYFVAKNQ GTVYGGTLYY QHNETYISLP |
| | CKLRPCSLSY |
| 551 | VPTEIPVLFS GNLSYTHTDN DLKTKYTTYP TVKGSWGNDS |
| | FALEFGGRAP |
| 601 | ICLDESALFE QYMPFMKLQF VYAHQEGFKE QGTEAREFGS |
| | SRLVNLALPI |
| 651 | GIRFDKESDC QDATYNLTLG YTVDLVRSNP DCTTTLRISG |
| | DSWKTFGTNL |
| 701 | ARQALVLRAG VHFCFNSNFE AFSQFSFELR GSSRNYNVDL |
| | GAKYQF* |

This sequence is frame-shifted with respect to cp0014.

The cp0015 nucleotide sequence <SEQ ID 20> is:

| | |
|---|---|
| 1 | ATGTCAGCTC TGTTTTCTGA AAATACCTCC TCAAAGAAAG |
| | GCGGAGCCAT |
| 51 | TCAGACTTCC GATGCCCTTA CCATTACTGG AAACCAAGGG |
| | GAAGTCTCTT |
| 101 | TTTCTGACAA TACTTCTTCG GATTCTGGAG CTGCAATTTT |
| | TACAGAAGCC |
| 151 | TCGGTGACTA TTTCTAATAA TGCTAAAGTT TCCTTTATTG |
| | ACAATAAGGT |
| 201 | CACAGGAGCG AGCTCCTCAA CAACGGGGGA TATGTCAGGA |
| | GGTGCTATCT |
| 251 | GTGCTTATAA AACTAGTACA GATACTAAGG TCACCCTCAC |
| | TGGAAATCAG |
| 301 | ATGTTACTCT TCAGCAACAA TACATCGACA ACAGCGGGAG |
| | GAGCTATCTA |
| 351 | TGTGAAAAAG CTCGAACTGG CTTCCGGAGG ACTTACCCTA |
| | TTCAGTAGAA |
| 401 | ATAGTGTCAA TGGAGGTACA GCTCCTAAAG GTGGAGCCAT |
| | AGCTATCGAA |
| 451 | GATAGTGGGG AATTGAGTTT ATCCGCCGAT AGTGGTGACA |
| | TTGTCTTTTT |
| 501 | AGGGAATACA GTCACTTCTA CTACTCCTGG GACGAATAGA |
| | AGTAGTATCG |
| 551 | ACTTAGGAAC GAGTGCAAAG ATGACAGCTT TGCGTTCTGC |
| | TGCTGGTAGA |
| 601 | GCCATCTACT TCTATGATCC CATAACTACA GGATCATCCA |
| | CAACAGTTAC |
| 651 | AGATGTCTTA AAAGTTAATG AGACTCCGGC AGATTCTGCA |
| | CTACAATATA |
| 701 | CAGGGAACAT CATCTTCACA GGAGAAAAGT TATCAGAGAC |
| | AGAGGCCGCA |
| 751 | GATTCTAAAA ATCTTACTTC GAAGCTACTA CAGCCTGTAA |
| | CTCTTTCAGG |
| 801 | AGGTACTCTA TCTTTAAAAC ATGGAGTGAC TCTGCAGACT |
| | CAGGCATTCA |
| 851 | CTCAACAGGC AGATTCTCGT CTCGAAATGG ACGTAGGAAC |
| | TACTCTAGAA |
| 901 | CCTGCTGATA CTAGCACCAT AAACAATTTG GTCATTAACA |
| | TCAGTTCTAT |
| 951 | AGACGGTGCA AAGAAGGCAA AAATAGAAAC CAAAGCTACG |
| | TCAAAAAATC |
| 1001 | TGACTTTATC TGGAACCATC ACTTTATTGG ACCCGACGGG |
| | CACGTTTTAT |
| 1051 | GAAAATCATA GTTTAAGAAA TCCTCAGTCC TACGACATCT |
| | TAGAGCTCAA |
| 1101 | AGCTTCTGGA ACTGTAACAA GCACCGCAGT GACTCCAGAT |
| | CCTATAATGG |
| 1151 | GTGAGAAATT CCATTACGGC TATCAGGGAA CTTGGGGCCC |
| | AATTGTTTGG |
| 1201 | GGGACAGGGG CTTCTACGAC TGCAACCTTC AACTGGACTA |
| | AAACTGGCTA |
| 1251 | TATTCCTAAT CCCGAGCGTA TCGGCTCTTT AGTCCCTAAT |
| | AGCTTATGGA |
| 1301 | ATGCATTTAT AGATATTAGC TCTCTCCATT ATCTTATGGA |
| | GACTGCAAAC |
| 1351 | GAAGGGTTGC AGGGAGACCG TGCTTTTTGG TGTGCTGGAT |
| | TATCTAACTT |
| 1401 | CTTCCATAAG GATAGTACAA AAACACGACG CGGGTTTCGC |
| | CATTTGAGTG |
| 1451 | GCGGTTATGT CATAGGAGGA AACCTACATA CTTGTTCAGA |
| | TAAGATTCTT |
| 1501 | AGTGCTGCAT TTTGTCAGCT CTTTGGAAGA GATAGAGACT |
| | ACTTTGTAGC |
| 1551 | TAAGAATCAA GGTACAGTCT ACGGAGGAAC TCTCTATTAC |
| | CAGCACAACG |
| 1601 | AAACCTATAT CTCTCTTCCT TGCAAACTAC GGCCTTGTTC |
| | GTTGTCTTAT |
| 1651 | GTTCCTACAG AGATTCCTGT TCTCTTTTCA GGAAACCTTA |
| | GCTACACCCA |
| 1701 | TACGGATAAC GATCTGAAAA CCAAGTATAC AACATATCCT |
| | ACTGTTAAAG |
| 1751 | GAAGCTGGGG GAATGATAGT TTCGCTTTAG AATTCGGTGG |
| | AAGAGCTCCG |
| 1801 | ATTTGCTTAG ATGAAAGTGC TCTATTTGAG CAGTACATGC |
| | CCTTCATGAA |
| 1851 | ATTGCAGTTT GTCTATGCAC ATCAGGAAGG TTTTAAAGAA |
| | CAGGGAACAG |
| 1901 | AAGCTCGTGA ATCTGGAAGT AGCCGTCTTG TGAATCTTGC |
| | CTTACCTATC |
| 1951 | GGGATCCGAT TTGATAAGGA ATCAGACTGC CAAGATGCAA |
| | CGTACAATCT |
| 2001 | AACTCTTGGT TATACTGTGG ATCTTGTTCG TAGTAACCCC |
| | GACTGTACGA |
| 2051 | CAACACTGCG AATTAGCGGT GATTCTTGGA AAACCTTCGG |
| | TACGAATTTG |
| 2101 | GCAAGACAAG CTTTAGTCCT TCGTGCAGGG AACCATTTTT |
| | GCTTTAACTC |
| 2151 | AAATTTTGAA GCCTTTAGCC AATTTTCTTT TGAATTGCGT |
| | GGGTCATCTC |
| 2201 | GCAATTACAA TCTAGACTTA GGAGCAAAAT ACCAATTCTA A |

The PSORT algorithm predicts a cytoplasmic location (0.274).

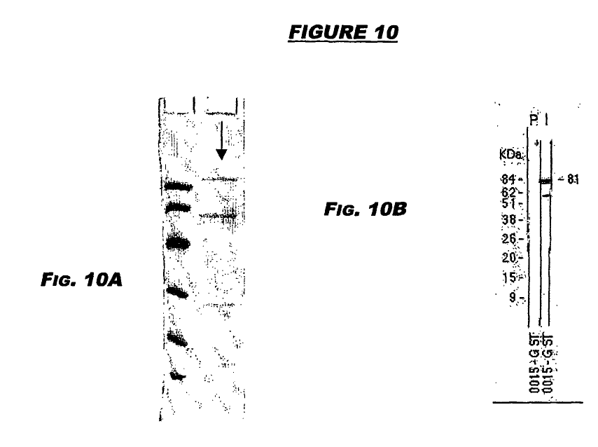

The protein was expressed in E. coli and purified as a GST-fusion product, as shown in FIG. 10A. The recombinant protein was used to immunise mice, whose sera were used in a Western blot (FIG. 10B) and for FACS analysis. A his-tagged protein was also expressed.

These experiments show that cp0015 is a useful immunogen. These properties are not evident from the sequence alone.

### Example 11

The following C. pneumoniae protein (PID 6172325) was expressed <SEQ ID 21; cp0019>:

| | |
|---|---|
| 1 | LQDSQDYSFV KLSPGAGGTI ITQDASQKPL EVAPSRFHYG |
| | YQGHWNVQVI |
| 51 | PGTGTQPSQA NLEWVRTGYL PNPERQGSLV PNSLWGSFVD |
| | QRAIQEIMVN |
| 101 | SSQILCQERG VWGAGIANFL HRDKINEHGY RHSGVGYLVG |
| | VGTHAFSDAT |
| 151 | INAAFCQLFS RDKDYVVSKN HGTSYSGVVF LEDTLEFRSP |
| | QGFYTDSSSE |
| 201 | ACCNQVVTID MQLSYSHRNN DMKTKYTTYP EAQGSWANDV |
| | FGLEFGATTY |
| 251 | YYPNSTFLFD YYSPFLRLQC TYAHQEDFKE TGGEVRHFTS |
| | GDLFNLAVPI |
| 301 | GVKFERFSDC KRGSYELTLA YVPDVIRKDP KSTATLASGA |
| | TWSTHGNNLS |
| 351 | RQGLQLRLGN HCLINPGIEV FSHGAIELRG SSRNYNINLG |
| | GKYRF* |

This sequence is frame-shifted with respect to cp0018.

The cp0019 nucleotide sequence <SEQ ID 22> is:

| | |
|---|---|
| 1 | TTGCAAGACT CTCAAGACTA TAGCTTTGTA AAGTTATCTC |
| | CAGGAGCGGG |
| 51 | AGGGACTATA ATTACTCAAG ATGCTTCTCA GAAGCCTCTT |
| | GAAGTAGCTC |
| 101 | CTTCTAGACC ACATTATGGC TATCAAGGAC ATTGGAATGT |
| | GCAAGTCATC |
| 151 | CCAGGAACGG GAACTCAACC GAGCCAGGCA AATTTAGAAT |
| | GGGTGCGGAC |
| 201 | AGGATACCTT CCGAATCCCG AACGGCAAGG ATCTTTAGTT |
| | CCCAATAGCC |
| 251 | TGTGGGGTTC TTTTGTTGAT CAGCGTGCTA TCCAAGAAAT |
| | CATGGTAAAT |
| 301 | AGTAGCCAAA TCTTATGTCA GGAACGGGGA GTCTGGGGAG |
| | CTGGAATTGC |
| 351 | TAATTTCCTA CATAGAGATA AAATTAATGA GCACGGCTAT |
| | CGCCATAGCG |
| 401 | GTGTCGGTTA TCTTGTGGGA GTTGGCACTC ATGCTTTTTC |
| | TGATGCTACG |
| 451 | ATAAATGCGG CTTTTTGCCA GCTCTTCAGT AGAGATAAAG |
| | ACTACGTAGT |
| 501 | ATCCAAAAAT CATGGAACTA GCTACTCAGG GGTCGTATTT |
| | CTTGAGGATA |
| 551 | CCCTAGAGTT TAGAAGTCCA CAGGGATTCT ATACTGATAG |
| | CTCCTCAGAA |
| 601 | GCTTGCTGTA ACCAAGTCGT CACTATAGAT ATGCAGTTGT |
| | CTTACAGCCA |
| 651 | TAGAAATAAT GATATGAAAA CCAAATACAC GACATATCCA |
| | GAAGCTCAGG |
| 701 | GATCTTGGGC AAATGATGTT TTTGGTCTTG AGTTTGGAGC |
| | GACTACATAC |
| 751 | TACTACCCTA ACAGTACTTT TTTATTTGAT TACTACTCTC |
| | CGTTTCTCAG |
| 801 | GCTGCAGTGC ACCTATGCTC ACCAGGAAGA CTTCAAAGAG |
| | ACAGGAGGTG |
| 851 | AGGTTCGTCA CTTTACTAGC GGAGATCTTT TCAATTTAGC |
| | AGTTCCTATT |
| 901 | GGCGTGAAGT TTGAGAGATT TTCAGACTGT AAAAGGGGAT |
| | CTTATGAACT |
| 951 | TACCCTTGCT TATGTTCCTG ATGTGATTCG CAAAGATCCC |
| | AAGAGCACGG |
| 1001 | CAACATTGGC TAGTGGAGCT ACGTGGAGCA CCCACGGAAA |
| | CAATCTCTCC |
| 1051 | AGACAAGGAT TACAACTGCG TTTAGGGAAC CACTGTCTCA |
| | TAAATCCTGG |
| 1101 | AATTGAGGTG TTCAGTCACG GAGCTATTGA ATTGCGGGGA |
| | TCCTCTCGTA |
| 1151 | ATTATAACAT CAATCTCGGG GGTAAATACC GATTTTAA |

The PSORT algorithm predicts a cytoplasmic location (0.189).

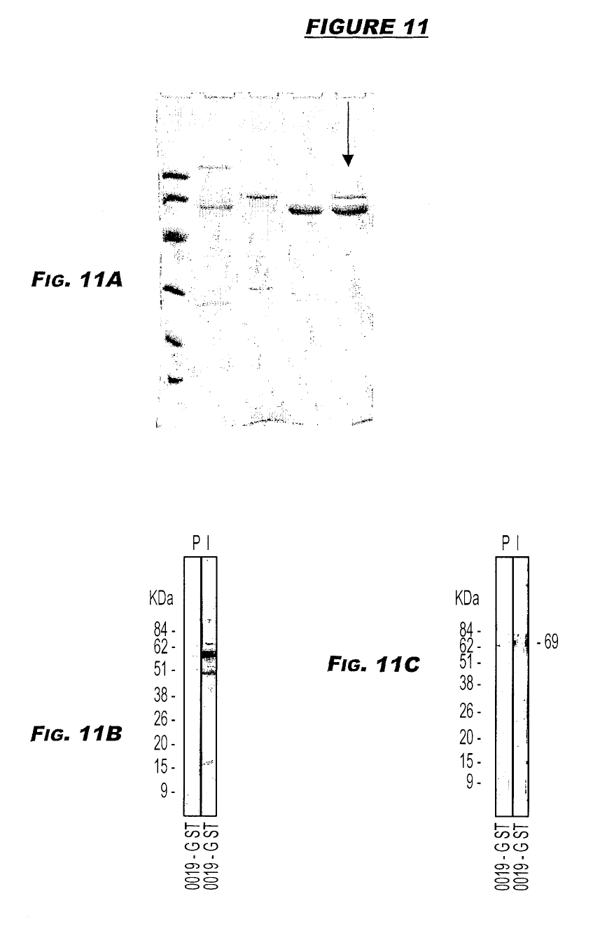

The protein was expressed in E. coli and purified as a GST-fusion product, as shown in FIG. 11A. This protein was used to immunise mice, whose sera were used in a Western blot (FIG. 11B) and an immunoblot assay (FIG. 11C). A his-tagged protein was also expressed.

These experiments show that cp0019 is a useful immunogen. These properties are not evident from the sequence alone.

### Example 12

The following C. pneumoniae protein (PID 4376466) was expressed <SEQ ID 23; cp6466>:

| | |
|---|---|
| 1 | MRKISVGICI TILLSLSVVL QGCKESSHSS TSRGELAINI |
| | RDEPRSLDPR |
| 51 | QVRLLSEISL VKHIYEGLVQ ENNLSGNIEP ALAEDYSLSS |
| | DGLTYTFKLK |
| 101 | SAFWSNGDPL TAEDFIESWK QVATQEVSGI YAFALNPIKN |
| | VRKIQEGHLS |
| 151 | IDHFGVHSPN ESTLVVTLES PTSHFLKLLA LPVFFPVEKS |
| | QRTLQSKSLP |
| 201 | IASGAFYPKN IKQKQWIKLS KNPHYYNQSQ VETKTITIHF |
| | IPDANTAAKL |
| 251 | FNQGKLNWQG PPWGERIPQE TLSNLQSKGH LHSFDVAGTS |
| | WLTFNINKFP |
| 301 | LNNMKLREAL ASALDKEALV STIFLGRAKT ADHLLPTNIH |
| | SYPEHQKQEM |
| 351 | AQRQAYAKKL FKAELEELQI TAKDLEHLNL IFPVSSSASS |
| | LLVQLIREQW |
| 401 | KESLGFAIPI VGKEFALLQA DLSSGNFSLA TGGWFADFAD |
| | PMAFLTIFAY |
| 451 | PSGVPPYAIN HKDFIEILQN IEQEQDHQKR SELVSQASLY |
| | LETFHIIEPI |
| 501 | YHDAFQFAMN KKLSNLGVSP TGVVDFRYAK EN* |

A predicted signal peptide is highlighted.

The cp6466 nucleotide sequence <SEQ ID 24> is:

| | |
|---|---|
| 1 | ATGCGCAAGA TATCAGTGGG AATCTGTATC ACCATTCTCC |
| | TTAGCCTCTC |
| 51 | CGTAGTCCTC CAAGGCTGCA AGGAGTCCAG TCACTCCTCT |
| | ACATCTCGGG |
| 101 | GAGAACTCGC TATTAATATA AGAGATGAAC CCCGTTCTTT |
| | AGATCCAAGA |
| 151 | CAAGTGCGAC TTCTTTCAGA AATCAGCCTT GTCAAACATA |
| | TCTATGAGGG |
| 201 | ATTAGTTCAA GAAAATAATC TTTCAGGAAA TATAGAGCCT |
| | GCTCTTGCAG |
| 251 | AAGACTACTC TCCTTCCTCG GACGGACTCA CTTATACTTT |
| | TAAACTGAAA |
| 301 | TCAGCTTTTT GGAGTAATGG CGACCCCTTA ACAGCTGAAG |
| | ACTTTATAGA |
| 351 | ATCTTGGAAA CAAGTAGCTA CTCAAGAAGT CTCAGGAATC |
| | TATGCTTTTG |
| 401 | CCTTGAATCC AATTAAAAAT GTACGAAAGA TCCAAGAGGG |
| | ACACCTCTCC |
| 451 | ATAGACCATT TTGGAGTGCA CTCTCCTAAT GAATCTACAC |
| | TTGTTGTTAC |
| 501 | CCTGGAATCC CCAACCTCGC ATTTCTTAAA ACTTTTAGCT |
| | CTTCCAGTCT |
| 551 | TTTTCCCCGT TCATAAATCT CAAAGAACCC TGCAATCCAA |
| | ATCTCTACCT |
| 601 | ATAGCAAGCG GAGCTTTCTA TCCTAAAAAT ATCAAACAAA |
| | AACAATGGAT |
| 651 | AAAACTCTCA AAAAACCCTC ACTACTATAA TCAAAGTCAG |
| | GTGGAAACTA |
| 701 | AAACGATTAC GATTCACTTC ATTCCCGATG CAAACACAGC |
| | AGCAAAACTA |
| 751 | TTTAATCAGG GAAAACTCAA TTGGCAAGGA CCTCCTTGGG |
| | GAGAACGCAT |
| 801 | TCCTCAAGAA ACCCTATCCA ATTTACAGTC TAAGGGGCAC |
| | TTACACTCTT |
| 851 | TTGATGTCGC AGGAACCTCA TGGCTCACCT TCAATATCAA |
| | TAAATTCCCC |
| 901 | CTCAACAATA TGAAGCTTAG AGAAGCCTTA GCATCAGCCT |
| | TAGATAAGGA |
| 951 | AGCTCTTGTC TCAACTATAT TCTTAGGCCG TGCAAAAACT |
| | GCCGATCATC |
| 1001 | TCCTACCTAC AAATATTCAT AGCTATCCCG AACATCAAAA |
| | ACAAGAGATG |
| 1051 | GCACAACGCC AAGCTTACGC TAAAAAACTC TTTAAAGAAG |
| | CTTTAGAAGA |
| 1101 | ACTCCAAATC ACTGCTAAAG ATCTCGAACA TCTTAATCTT |
| | ATCTTTCCCG |
| 1151 | TTTCCTCGTC AGCAAGTTCT TTACTAGTCC AACTTATACG |
| | AGAACAGTGG |
| 1201 | AAAGAAAGTT TAGGGTTCGC TATCCCTATT GTCGGAAAGG |
| | AATTTGCTCT |
| 1251 | TCTCCAAGCA GACCTATCTT CAGGGAACTT CTCTTTAGCT |
| | ACAGGAGGAT |
| 1301 | GGTTCGCAGA CTTTGCTGAT CCTATGGCAT TTCTAACGAT |
| | CTTTGCTTAT |
| 1351 | CCATCAGGAG TTCCTCCTTA TGCAATCAAC CATAAGGACT |
| | TCCTAGAAAT |
| 1401 | TCTACAAAAC ATAGAACAAG AGCAAGATCA CCAAAAACGC |
| | TCGGAATTAG |
| 1451 | TGTCGCAAGC TTCTCTTTAC CTAGAGACCT TTCATATTAT |
| | TGAGCCGATC |
| 1501 | TACCACGACG CATTTCAATT TGCTATGAAT AAAAAACTTT |
| | CTAATCTAGG |
| 1551 | AGTCTCACCA ACAGGAGTTG TGGACTTCCG TTATGCTAAG |
| | GAAAATTAG |

The PSORT algorithm predicts that the protein is an outer membrane lipoprotein (0.790).

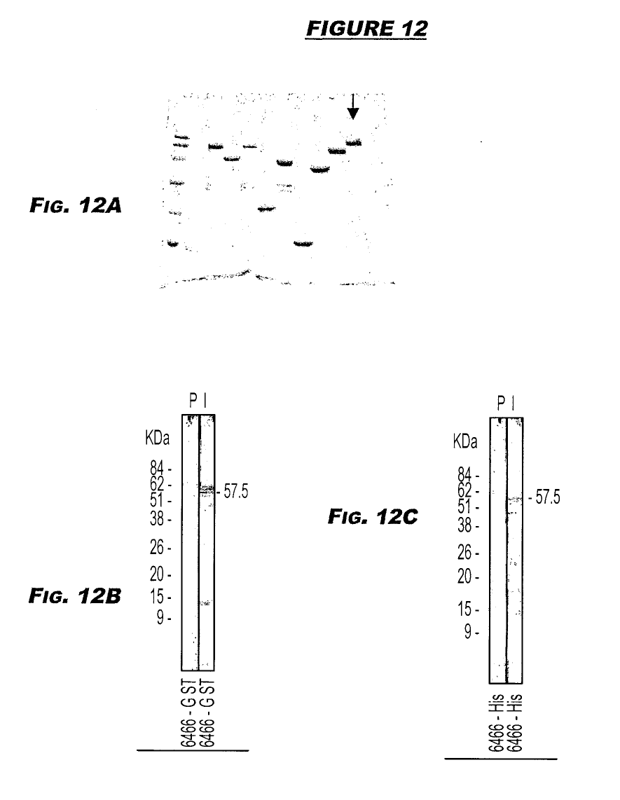

The protein was expressed in E. coli and purified both as a GST-fusion product and a His-tag fusion product. Purification of the protein as a GST-fusion product is shown in FIG. 12A. The recombinant proteins were used to immunise mice, whose sera were used in Western blots (FIGS. 12B and 12C). FACS analysis was also performed.

These experiments show that cp6466 is a useful immunogen. These properties are not evident from the sequence alone.

### Example 13

The following C. pneumoniae protein (PID 4376468) was expressed <SEQ ID 25; cp6468>:

| | |
|---|---|
| 1 | MFSRWITLFL LFISLTGCSS YSSKHKQSLI IPIHDDPVAF |
| | SPEQAKRAMD |
| 51 | LSIAQLLFDG ITRETHRESN DLELAIASRY TVSEDFCSYT |
| | FFIKDSALWS |
| 101 | DGTPITSEDI RNAWEYAQEN SPHIQIFQGL NFSTPSSNAI |
| | TIHLDSPNPD |
| 151 | FPKLLAFPAF AIFKPENPKL FSGPYTLVEY FPGENIHLKK |
| | NPNYYDYHCV |
| 201 | SINSIKLLII PDIYTAIHLL NRGKVDWVGQ PWHQGIPWEL |
| | HKQSQYHYYT |
| 251 | YPVEGAFWLC INTKSPHLND LQNRHRLATC IDKRSIIEEA |
| | LQGTQQPAET |
| 301 | LSRGAPQPNQ YKKQKPLTPQ EKLVLTYPSD ILRCQRIAEI |
| | LKEQWKAAGI |
| 351 | DLILEGLEYH LFVNKRKVQD YAIATQTGVA YYPGANLISE |
| | EDKLLQNFEI |
| 401 | IPIYYLSYDY LTQDFIEGVI YNASGAVDLK YTYFP* |

A predicted signal peptide is highlighted.

The cp6468 nucleotide sequence <SEQ ID 26> is:

| | |
|---|---|
| 1 | ATGTTTTCAC GATGGATCAC CCTCTTTTTA TTATTCATTA |
| | GCCTTACTGG |
| 51 | ATGCTCCTCC TACTCTTCAA AACATAAACA ATCTTTAATT |
| | ATTCCCATAC |
| 101 | ATGACGACCC TCTAGCTTTT TCTCCTGAAC AAGCAAAACG |
| | GGCCATGGAC |
| 151 | CTTTCTATTG CCCAACTTCT TTTTGATGGT CTGACTAGAG |
| | AAACTCATCG |
| 201 | CGAATCCAAT GATTTGGAAT TAGCGATTGC CAGTCGCTAT |
| | ACAGTCTCTG |
| 251 | AAGACTTTTG CTCTTATACG TTCTTTATCA AAGACAGCGC |
| | TTTATGGAGC |
| 301 | GACGGAACAC CAATCACCTC CGAAGATATC CGTAACGCTT |
| | GGGAGTATGC |
| 351 | ACAGGAGAAC TCTCCCCACA TACAGATCTT CCAAGGACTT |
| | AACTTCTCAA |
| 401 | CTCCTTCATC AAATGCAATT ACGATTCATC TCGACTCGCC |
| | CAACCCCGAT |
| 451 | TTTCCTAAGC TTCTTGCCTT TCCTGCATTT GCTATCTTTA |
| | AACCAGAAAA |
| 501 | CCCGAAGCTC TTTAGCGGTC CGTATACTCT TGTAGAGTAT |
| | TTCCCAGGGC |
| 551 | ATAACATTCA TTTAAAGAAA AACCCTAACT ATTACGACTA |
| | CCACTGCGTC |
| 601 | TCCATCAACT CCATCAAACT GCTCATTATT CCTGATATAT |
| | ATACAGCCAT |
| 651 | CCACCTCCTA AACAGAGGCA AGGTGGACTG GGTAGGACAA |
| | CCCTGGCATC |
| 701 | AAGGGATTCC TTGGGAGCTC CATAAACAAT CGCAATATCA |
| | CTACTACACC |
| 751 | TATCCTGTAG AAGGTGCCTT CTGGCTTTGT CTAAATACAA |
| | AATCCCCACA |
| 801 | CTTAAATGAT CTTCAAAACA GACATAGACT CGCTACTTGT |
| | ATTGATAAAC |
| 851 | GTTCTATCAT TGAAGAAGCT CTTCAAGGAA CCCAACAACC |
| | AGCGGAAACA |
| 901 | CTGTCCCGAG GAGCTCCACA ACCAAATCAA TATAAAAAAC |
| | AAAAGCCTCT |
| 951 | AACTCCACAA GAAAAACTCG TGCTTACCTA TCCCTCAGAT |
| | ATTCTAAGAT |
| 1001 | GCCAACGCAT AGCAGAAATC TTAAAGGAAC AATGGAAAGC |
| | TGCTGGAATA |
| 1051 | GATTTAATCC TTGAAGGACT CGAATACCAT CTGTTTGTTA |
| | ACAAACGAAA |
| 1101 | AGTCCAAGAC TACGCCATAG CAACACAGAC TGGAGTTGCT |
| | TATTACCCAG |
| 1151 | GAGCAAATCT AATTTCTGAA GAAGACAAGC TCCTGCAAAA |
| | CTTTGAGATT |
| 1201 | ATCCCGATCT ACTATCTGAG CTATGACTAT CTCACTCAAG |
| | ATTTTATAGA |
| 1251 | GGGAGTAATC TATAATGCTT CTGGAGCTGT AGATCTCAAA |
| | TATACCTATT |
| 1301 | TCCCCTAG |

The PSORT algorithm predicts that this protein is an outer membrane lipoprotein (0.790).

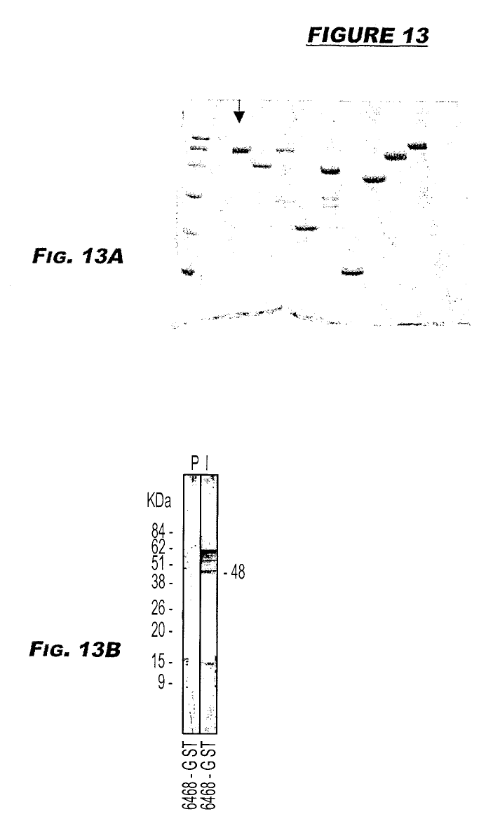

The protein was expressed in E. coli and purified as a GST-fusion product, as shown in FIG. 13A. The recombinant protein was used to immunise mice, whose sera were used in a Western blot (FIG. 13B) and for FACS analysis. A his-tagged protein was also expressed.

These experiments show that cp6468 is a useful immunogen. These properties are not evident from the sequence alone.

### Example 14

The following C. pneumoniae protein (PID 4376469) was expressed <SEQ ID 27; cp6469>:

| | |
|---|---|
| 1 | MKMHRLKPTL KSLIPNLLFL LLTLSSCSKQ KQEPLGKELV |
| | IAMSHDLADL |
| 51 | DPRNAYLSRD ASLAKALYEG LTRETDQGIA LALAESYTLS |
| | KDHKVYTFKL |
| 101 | RPSVWSDGTP LTAYDFEKSI KQLYFEEFSP SIHTLLGVIK |
| | NSSAIHNAQK |
| 151 | SLETLGIQAK DDLTLVITLE QPFPYFLTLI ARPVFSPVHH |
| | TLRESYKKGT |
| 201 | PPSTYISNGP FVLKKHEHQN YLILEKNPHY YDHESVKLDR |
| | VTLKIIPDAS |
| 251 | TATKLFKSKS IDWIGSPWSA PISNEDQKVL SQEKILTYSV |
| | SSTTLLIYNL |
| 301 | QKPLIQNKAL RKAIAHAIDR KSILRLVPSG QEAVTLVPPN |
| | LSQLNLQKEI |
| 351 | STEFRQTKAR AYFQEAKETL SEKELAELSI LYPIDSSNSS |
| | IIAQEIQRQL |
| 401 | KDTLGLKIKI QGMEYHCFLK KRRQGDFFIA TGGWIAEYVS |
| | PVAFLSILGN |
| 451 | PRDLTQWRNS DYEKTLEKLY LPHAYKENLK RAEMIIEEET |
| | PIIPLYHGKY |
| 501 | IYAIHPKIQN TFGSLLGHTD LKNIDILS* |

A predicted signal peptide is highlighted.

The cp6469 nucleotide sequence <SEQ ID 28> is:

| | |
|---|---|
| 1 | ATGAAGATGC ATAGGCTTAA ACCTACCTTA AAAAGTCTGA |
| | TCCCTAATCT |
| 51 | TCTTTTCTTA TTGCTCACTC TTTCAAGCTG CTCAAAGCAA |
| | AAACAAGAAC |
| 101 | CCTTAGGAAA ACATCTCGTT ATTGCGATGA GCCATGATCT |
| | CGCCGACCTA |
| 151 | GATCCTCGCA ATGCCTATTT AAGCAGAGAT GCTTCCCTAG |
| | CAAAAGCCCT |
| 201 | CTATGAAGGA CTGACAAGAG AAACTGATCA AGGAATCGCA |
| | CTGGCTCTTG |
| 251 | CAGAAAGTTA TACCCTGTCA AAAGATCATA AGGTCTATAC |
| | CTTTAAACTC |
| 301 | AGACCTTCTG TGTGGAGCGA TGGCACTCCA CTCACTGCTT |
| | ATGACTTTGA |
| 351 | AAAATCTATA AAACAACTGT ACTTCGAAGA ATTTTCACCT |
| | TCCATACATA |
| 401 | CTTTACTCGG CGTGATTAAA AATTCTTCGG CAATCCACAA |
| | TGCTCAAAAA |
| 451 | TCTCTGGAAA CTCTTGGGAT ACAGGCAAAA GATGATCTTA |
| | CTTTGGTGAT |
| 501 | TACCCTAGAG CAACCTTTCC CATACTTTCT CACACTTATC |
| | GCTCGCCCCG |
| 551 | TATTCTCCCC TGTTCATCAC ACCCTTAGGG AATCCTATAA |
| | GAAAGGAACA |
| 601 | CCCCCATCCA CATACATCTC CAATGGGCCC TTTGTCTTAA |
| | AAAAACATGA |
| 651 | ACACCAAAAC TACTTAATTT TAGAAAAAAA TCCTCACTAC |
| | TATGATCATG |
| 701 | AATCAGTAAA GTTAGACCGA GTCACCTTAA AAATTATCCC |
| | AGACGCCTCC |
| 751 | ACAGCCACGA AACTTTTCAA AAGTAAATCT ATAGATTGGA |
| | TTGGCTCACC |
| 801 | TTGGAGCGCT CCGATATCTA ACGAAGACCA AAAAGTTCTC |
| | TCCCAAGAAA |
| 851 | AGATTCTTAC CTATTCTGTT TCAAGCACCA CCCTTCTTAT |
| | CTATAACCTG |
| 901 | CAAAAACCTC TAATACAAAA TAAAGCCCTC AGGAAAGCCA |
| | TTGCTCATGC |
| 951 | TATTGATAGA AAATCTATCT TAAGACTCGT GCCTTCAGGA |
| | CAAGAAGCTG |
| 1001 | TAACTCTAGT TCCCCCAAAT CTTTCACAAC TCAATCTTCA |
| | AAAAGAGATC |
| 1051 | TCAACAGAAG AACGACAAAC AAAAGCCAGA GCATATTTTC |
| | AAGAAGCTAA |
| 1101 | AGAAACACTT TCTGAAAAAG AACTCGCAGA ACTCAGCATC |
| | CTCTATCCTA |
| 1151 | TAGATTCCTC GAATTCCTCC ATCATAGCTC AAGAAATCCA |
| | AAGACAACTT |
| 1201 | AAAGATACCT TAGGATTGAA AATCAAAATC CAAGGCATGG |
| | AGTACCACTG |
| 1251 | CTTTTTAAAG AAACGTCGTC AAGGAGATTT CTTCATAGCG |
| | ACAGGAGGAT |
| 1301 | GGATTGCGGA ATACGTAAGC CCCGTAGCCT TCCTATCTAT |
| | TCTAGGCAAC |
| 1351 | CCCAGAGACC TCACACAATG GAGAAACAGT GATTACGAAA |
| | AGACTTTAGA |
| 1401 | GAAACTCTAT CTCCCTCATG CCTACAAAGA GAATTTAAAA |
| | CGCGCAGAAA |
| 1451 | TGATAATAGA AGAAGAAACC CCGATTATCC CCCTGTATCA |
| | CGGCAAATAT |
| 1501 | ATTTACGCTA TACATCCTAA AATCCAGAAT ACATTCGGAT |
| | CTCTTCTAGG |
| 1551 | CCACACAGAT CTCAAAAATA TCGATATCTT AAGTTAG |

The PSORT algorithm predicts a periplasmic location (0.934).

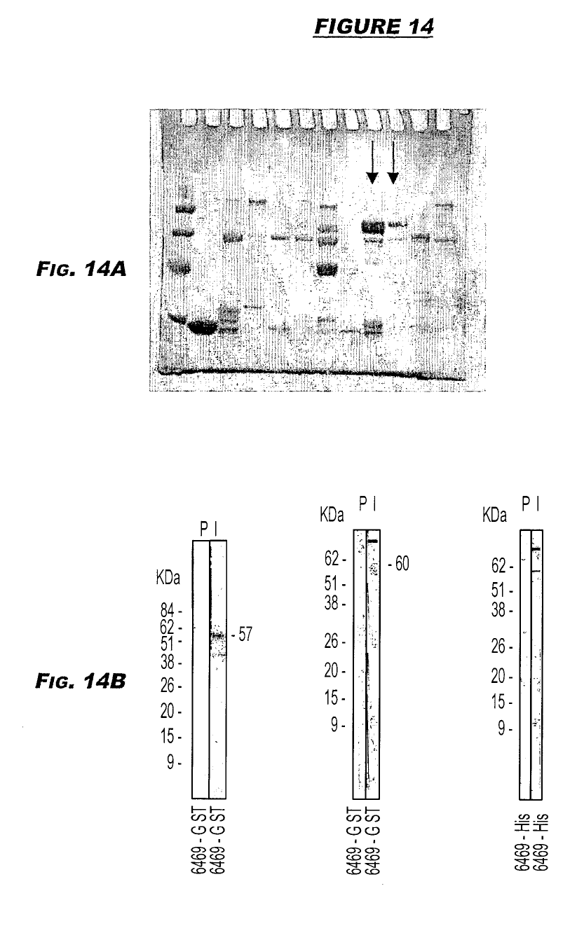

The protein was expressed in E. coli and purified as a GST-fusion product, as shown in FIG. 14A. The recombinant protein was used to immunise mice, whose sera were used in a Western blot (FIG. 14B) and for FACS analysis. A his-tagged protein was also expressed.

These experiments show that cp6469 is a useful immunogen. These properties are not evident from the sequence alone.

### Example 15

The following C. pneumoniae protein (PID 4376602) was expressed <SEQ ID 29; cp6602>:

| | |
|---|---|
| 1 | MAASGGTGGL GGTQGVNLAA VEAAAAKADA AEVVASQEGS |
| | EMNMIQQSQD |
| 51 | LTNPAAATRT KKKEEKFQTL ESRKKGEAGK AEKKSESTEE |
| | KPDTDLADKY |
| 101 | ASGNSEISGQ ELRGLRDAIG DDASPEDILA LVQEKIKDPA |
| | LQSTALDYLV |
| 151 | QTTPPSQGKL KEALIQARNT HTEQFGRTAI GAKNILFASQ |
| | FYADQLNVSP |
| 201 | SGLRSLYLEV TGDTHTCDQL LSMLQDRYTY QDMAIVSSFL |
| | MKGMATELKR |
| 251 | QGPYVPSAQL QVLMTETRNL QAVLTSYDYF ESRVPILLDS |
| | LKAEGIQTPS |
| 301 | DLNFVKVAES YHKIINDKFP TASKVEREVR NLIGDDVDSV |
| | TGVLNLFFSA |
| 351 | LRQTSSRLFS SADKRQQLGA MIANALDAVN INNEDYPKAS |
| | DFPKPYPWS* |

The cp6602 nucleotide sequence <SEQ ID 30> is:

| | |
|---|---|
| 1 | ATGGCAGCAT CAGGAGGCAC AGGTGGTTTA GGAGGCACTC |
| | AGGGTGTCAA |
| 51 | CCTTGCAGCT GTAGAAGCTG CAGCTGCAAA AGCAGATGCA |
| | GCAGAAGTTG |
| 101 | TAGCCAGCCA AGAAGGTTCT GAGATGAACA TGATTCAACA |
| | ATCTCAGGAC |
| 151 | CTGACAAATC CCGCAGCAGC AACACGCACG AAAAAAAAGG |
| | AAGAGAAGTT |
| 201 | TCAAACTCTA GAATCTCGGA AAAAAGGAGA AGCTGGAAAG |
| | GCTGAGAAAA |
| 251 | AATCTGAATC TACAGAAGAG AAGCCTGACA CAGATCTTGC |
| | TGATAAGTAT |
| 301 | GCTTCTGGGA ATTCTGAAAT CTCTGGTCAA GAACTTCGCG |
| | GCCTGCGTGA |
| 351 | TGCAATAGGA GACGATGCTT CTCCAGAAGA CATTCTTGCT |
| | CTTGTACAAG |
| 401 | AGAAAATTAA AGACCCAGCT CTGCAATCCA CAGCTTTGGA |
| | CTACCTGGTT |
| 451 | CAAACGACTC CACCCTCCCA AGGTAAATTA AAAGAAGCGC |
| | TTATCCAAGC |
| 501 | AAGGAATACT CATACGGAGC AATTCGGACG AACTGCTATT |
| | GGTGCGAAAA |
| 551 | ACATCTTATT TGCCTCTCAA GAATATGCAG ACCAACTGAA |
| | TGTTTCTCCT |
| 601 | TCAGGGCTTC GCTCTTTGTA CTTAGAAGTG ACTGGAGACA |
| | CACATACCTG |
| 651 | TGATCAGCTA CTCTCTATGC TTCAAGACCG CTATACCTAC |
| | CAAGATATGG |
| 701 | CTATTGTCAG CTCCTTTCTA ATGAAAGGAA TGGCAACAGA |
| | ATTAAAAAGG |
| 751 | CAGGGTCCCT ACGTACCCAG TGCGCAACTA CAAGTTCTCA |
| | TGACAGAAAC |
| 801 | TCGTAACCTG CAAGCAGTTC TTACCTCGTA CGATTACTTT |
| | GAAAGTCGCG |
| 851 | TTCCTATTTT ACTCGATAGC TTAAAAGCTG AGGGAATCCA |
| | AACTCCTTCT |
| 901 | GATCTAAACT TTGTGAAGGT AGCTGAGTCC TACCATAAAA |
| | TCATTAACGA |
| 951 | TAAGTTCCCA ACAGCATCTA AAGTAGAACG AGAAGTCCGC |
| | AATCTCATAG |
| 1001 | GAGACGATGT TGATTCTGTG ACCGGTGTCT TGAACTTATT |
| | CTTTTCTGCT |
| 1051 | TTACGTCAAA CGTCGTCACG CCTTTTCTCT TCAGCAGACA |
| | AACGTCAGCA |
| 1101 | ATTAGGAGCT ATGATTGCTA ATGCTTTAGA TGCTGTAAAT |
| | ATAAACAATG |
| 1151 | AAGATTATCC CAAACGATCA GACTTCCCTA AACCCTATCC |
| | TTGGTCATGA |

The PSORT algorithm predicts a cytoplasmic location (0.080).

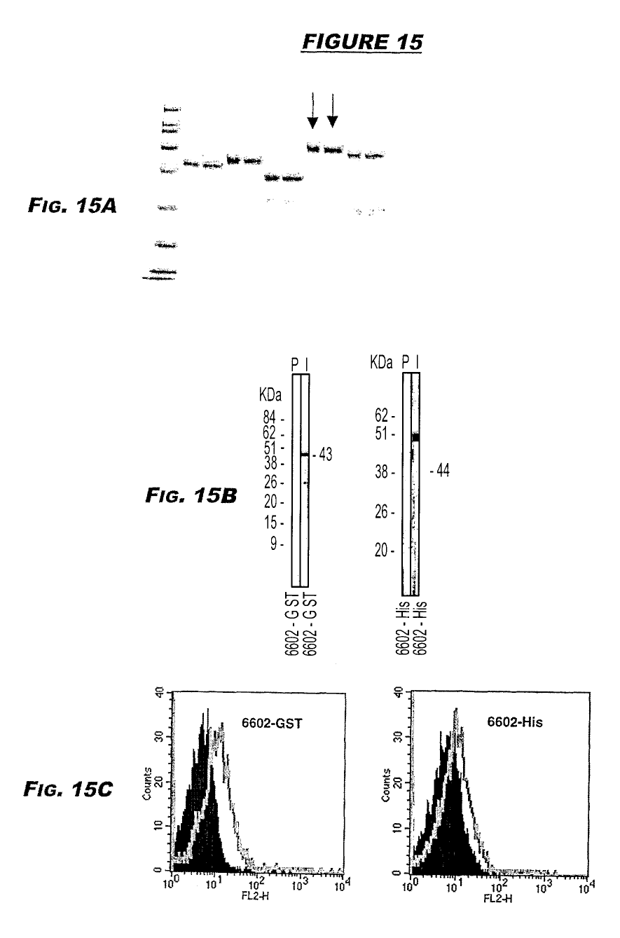

The protein was expressed in E. coli and purified as both a His-tag and a GST-fusion product, as shown in FIG. 15A. The recombinant proteins were used to immunise mice, whose sera were used in a Western blot (FIG. 15B) and for FACS analysis (FIG. 15C).

The cp6602 protein was also identified in the 2D-PAGE experiment (Cpn0324).

These experiments show that cp6602 is a surface-exposed and immunoaccessible protein, and that it is a useful immunogen. These properties are not evident from the sequence alone.

### Example 16

The following C. pneumoniae protein (PID 4376727) was expressed <SEQ ID 31; cp6727>:

| | |
|---|---|
| 1 | MKYSLPWLLT SSALVFSLHP LMAANTDLSS SDNYENGSSG |
| | SAAFTAKETS |
| 51 | DASGTTYTLT SDVSITNVSA ITPADKSCFT NTGGALSFVG |
| | ADHSLVLQTI |
| 101 | ALTHDGAAIN NTNTALSFSG FSSLLIDSAP ATGTSGGKGA |
| | ICVTNTEGGT |
| 151 | ATFTDNADVT LQKNTSEKDG AAVSAYSIDL AKITTAALLD |
| | QNTSTKNGGA |
| 201 | LCSTANTTVQ GNSGTVTFSS NTATDKGGGI YSKEKDSTLD |
| | ANTGVVTFKS |
| 251 | NTAKTGGAWS SDDNLALTGN TQVLFQENKT TGSAAQANNP |
| | EGCGGAICCY |
| 301 | LATATDKTGL AISQNQEMSF TSNTTTANGG AIYATKCTLD |
| | GNTTLTFDQN |
| 351 | TATAGCGGAI YTETEDFSLK GSTGTVTFST NTAKTGGALY |
| | SKGNSSLTGN |
| 401 | TNLLFSGNKA TGPSNSSANQ EGCGGAILAF IDSGSVSDKT |
| | GLSIANNQEV |
| 451 | SLTSNAATVS GGAIYATKCT LTGNGSLTFD GNTAGTSGGA |
| | IYTETEDFTL |
| 501 | TGSTGTVTFS TNTAKTGGAL YSKGNNSLSG NTNLLFSGNK |
| | ATGPSNSSAN |
| 551 | QEGCGGAILS FLESASVSTK KGLWIEDNEN VSLSGNTATV |
| | SGGAIYATKC |
| 601 | ALHGNTTLTF DGNTAETAGG AIYTETEDFT LTGSTGTVTF |
| | STNTAKTAGA |
| 651 | LHTKGNTSFT KNKALVFSGN SATATATTTT DQEGCGGAIL |
| | CNISESDIAT |
| 701 | KSLTLTENES LSFINNTAKR SGGGIYAPKC VISGSESINF |
| | DGNTAETSGG |
| 751 | AIYSKNLSIT ANGPVSFTNN SGGKGGAIYI ADSGELSLEA |
| | IDGDITFSGN |
| 801 | RATEGTSTPN SIHLGAGAKI TKLAAAPGHT IYFYDPITME |
| | APASGGTIEE |
| 851 | LVINPVVKAI VPPPQPKNGP IASVPVVPVA PANPNTGTIV |
| | FSSGKLPSQD |
| 901 | ASIPANTTTI LNQKINLAGG NVVLKEGATL QVYSFTQQPD |
| | STVFMDAGTT |
| 951 | LETTTTNNTD GSIDLKNLSV NLDALDGKRM ITIAVNSTSG |
| | GLKISGDLKF |
| 1001 | HNNEGSFYDN PGLKANLNLP FLDLSSTSGT VNLDDFNPIP |
| | SSMAAPDYGY |
| 1051 | QGSWTLVPKV GAGGKVTLVA EWQALGYTPK PELRATLVPN |
| | SLWNAYVNIH |
| 1101 | SIQQEIATAM SDAPSHPGIW IGGIGNAFHQ DKQKENAGFR |
| | LISRGYIVGG |
| 1151 | SMTTPQEYTF AVAFSQLFGK SKDYVVSDIK SQVYAGSLCA |
| | QSSYVIPLHS |
| 1201 | SLRRHVLSKV LPELPGETPL VLHGQVSYGR NHHNMTTKLA |
| | NNTQGKSDWD |
| 1251 | SHSFAVEVGG SLPVDLNYRY LTSYSPYVKL QVVSVNQKGF |
| | QEVAADPRIF |
| 1301 | DASHLVNVSI PMGLTFKHES AKPPSALLLT LGYAVDAYRD |
| | HPHCLTSLTN |
| 1351 | GTSWSTFATN LSRQAFFAEA SGHLKLLHGL DCFASGSCEL |
| | RSSSRSYNAN |
| 1401 | CGTRYSF* |

A predicted signal peptide is highlighted.

The cp6727 nucleotide sequence <SEQ ID 32> is:

| | |
|---|---|
| 1 | ATGAAATATT CTTTACCTTG GCTACTTACC TCTTCGGCTT |
| | TAGTTTTCTC |
| 51 | CCTACATCCA CTAATGGCTG CTAACACGGA TCTCTCATCA |
| | TCCGATAACT |
| 101 | ATGAAAATGG TAGTAGTGGT AGCGCAGCAT TCACTGCCAA |
| | GGAAACTTCG |
| 151 | GATGCTTCAG GAACTACCTA CACTCTCACT AGCGATGTTT |
| | CTATTACGAA |
| 201 | TGTATCTGCA ATTACTCCTG CAGATAAAAG CTGTTTTACA |
| | ACCACAGGAG |
| 251 | GAGCATTGAG TTTTGTTGGA GCTGATCACT CATTGGTTCT |
| | GCAAACCATA |
| 301 | GCGCTTACGC ATGATGGTGC TGCAATTAAC AATACCAACA |
| | CAGCTCTTTC |
| 351 | TTTCTCAGGA TTCTCGTCAC TCTTAATCGA CTCAGCTCCA |
| | GCAACAGGAA |
| 401 | CTTCGGGCGG CAAGGGTGCT ATTTGTGTGA CAAATACAGA |
| | GGGAGGTACT |
| 451 | GCGACTTTTA CTGACAATGC CAGTGTCACC CTCCAAAAAA |
| | ATACTTCAGA |
| 501 | AAAAGATGGA GCTGCAGTTT CTGCCTACAG CATCGATCTT |
| | GCTAAGACTA |
| 551 | CGACAGCAGC TCTCTTAGAT CAAAATACTA GCACAAAAAA |
| | TGGCGGGGCC |
| 601 | CTCTGTAGTA CAGCAAACAC TACAGTCCAA GGAAACTCAG |
| | GAACGGTGAC |
| 651 | CTTCTCCTCA AATACTGCTA CAGATAAAGG TGGGGGGATC |
| | TACTCAAAAG |
| 701 | AAAAGGATAG CACGCTAGAT GCCAATACAG GAGTCGTTAC |
| | CTTCAAATCT |
| 751 | AATACTGCAA AGACGGGGGG TGCTTGGAGC TCTGATGACA |
| | ATCTTGCTCT |
| 801 | TACCGGCAAC ACTCAAGTAC TTTTTCAGGA AAATAAAACA |
| | ACCGGCTCAG |
| 851 | CAGCACAGGC AAATAACCCG GAAGGTTGTG GTGGGGCAAT |
| | CTGTTGTTAT |
| 901 | CTTGCTACAG CAACAGACAA AACTGGATTA GCCATTTCTC |
| | AGAATCAAGA |
| 951 | AATGAGCTTC ACTAGTAATA CAACAACTGC GAATGGTGGA |
| | GCGATCTACG |
| 1001 | CTACTAAATG TACTCTGGAT GGAAACACAA CTCTTACCTT |
| | CGATCAGAAT |
| 1051 | ACTGCGACAG CAGGATGTGG CGGAGCTATC TATACAGAAA |
| | CTGAAGATTT |
| 1101 | TTCTCTTAAG GGAAGTACGG GAACCGTGAC CTTCAGCACA |
| | AATACAGCAA |
| 1151 | AGACAGGCGG CGCCTTATAT TCTAAAGGAA ACAGCTCGCT |
| | GACTGGAAAT |
| 1201 | ACCAACCTGC TCTTTTCAGG GAACAAAGCT ACGGGCCCGA |
| | GTAATTCTTC |
| 1251 | AGCAAATCAA GAGGGTTGCG GTGGGGCAAT CCTAGCCTTT |
| | ATTGATTCAG |
| 1301 | GATCCGTAAG CGATAAAACA GGACTATCGA TTGCAAACAA |
| | CCAAGAAGTC |
| 1351 | AGCCTCACTA GTAATGCTGC AACAGTAAGT GGTGGTGCGA |
| | TCTATGCTAC |
| 1401 | CAAATGTACT CTAACTGGAA ACGGCTCCCT GACCTTTGAC |
| | GGCAATACTG |
| 1451 | CTGGAACTTC AGGAGGGGCG ATCTATACAG AAACTGAAGA |
| | TTTTACTCTT |
| 1501 | ACAGGAAGTA CAGGAACCGT GACCTTCAGC ACAAATACAG |
| | CAAAGACAGG |
| 1551 | CGGCGCCTTA TATTCTAAAG GCAACAACTC TCTGTCTGGT |
| | AATACCAACC |
| 1601 | TGCTCTTTTC AGGGAACAAA GCTACGGGCC CGAGTAATTC |
| | TTCAGCAAAT |
| 1651 | CAAGAGGGTT GCGGTGGGGC AATCCTATCG TTTCTTGAGT |
| | CAGCATCTGT |
| 1701 | AAGTACTAAA AAAGGACTCT GGATTGAAGA TAACGAAAAC |
| | GTGAGTCTCT |
| 1751 | CTGGTAATAC TCGAACAGTA AGTGGCGGTG CGATCTATGC |
| | GACCAAGTGT |
| 1801 | GCTCTGCATG GAAACACGAC TCTTACCTTT GATGGCAATA |
| | CTGCCGAAAC |
| 1851 | TGCAGGAGGA GCGATCTATA CAGAAACCGA AGATTTTACT |
| | CTTACGGGAA |
| 1901 | GTACGGGAAC CGTGACCTTC AGCACAAATA CAGCAAAGAC |
| | AGCAGGGGCT |
| 1951 | CTACATACTA AAGGAAATAC TTCCTTTACC AAAAATAAGG |
| | CTCTTGTATT |
| 2001 | TTCTGGAAAT TCAGCAACAG CAACAGCAAC AACAACTACA |
| | GATCAAGAAG |
| 2051 | GTTGTGGTGG AGCGATCCTC TGTAATATCT CAGAGTCTGA |
| | CATAGCTACA |
| 2101 | AAAAGCTTAA CTCTTACTGA AAATGAGAGT TTAAGTTTCA |
| | TTAACAATAC |
| 2151 | GGCAAAAAGA AGTGGTGGTG GTATTTATGC TCCTAAGTGT |
| | GTAATCTCAG |
| 2201 | GCAGTGAATC CATAAACTTT GATGGCAATA CTGCTGAAAC |
| | TTCGGGAGGA |
| 2251 | GCGATTTATT CGAAAAACCT TTCGATTACA GCTAACGGTC |
| | CTGTCTCCTT |
| 2301 | TACCAATAAT TCTGGAGGCA AGGGAGGCGC CATTTATATA |
| | GCCGATAGCG |
| 2351 | GAGAACTTTC CTTAGAGGCT ATTGATGGGG ATATTACTTT |
| | CTCAGGGAAC |
| 2401 | CGAGCGACTG AGGGAACTTC AACTCCCAAC TCGATCCATT |
| | TAGGTGCAGG |
| 2451 | GGCTAAGATC ACTAAGCTTG CAGCAGCTCC TGGTCATACG |
| | ATTTATTTTT |
| 2501 | ATGATCCTAT TACGATGGAA GCTCCTGCAT CTGGAGGAAC |
| | AATAGAGGAG |
| 2551 | TTAGTCATCA ATCCTGTTGT CAAAGCTATT GTTCCTCCTC |
| | CCCAACCAAA |
| 2601 | AAATGGTCCT ATAGCTTCAG TGCCTGTAGT CCCTGTAGCA |
| | CCTGCAAACC |
| 2651 | CAAACACGGG AACTATAGTA TTTTCTTCTG GAAAACTCCC |
| | CAGTCAAGAT |
| 2701 | GCCTCGATTC CTGCAAATAC TACCACCATA CTGAACCAGA |
| | AGATCAACTT |
| 2751 | AGCAGGAGGA AATGTCGTTT TAAAAGAAGG AGCCACCCTA |
| | CAAGTATATT |
| 2801 | CCTTCACACA GCAGCCTGAT TCTACAGTAT TCATGGATGC |
| | AGGAACGACC |
| 2851 | TTAGAGACCA CGACAACTAA CAATACAGAT GGCAGCATCG |
| | ATCTAAAGAA |
| 2901 | TCTCTCTGTA AATCTGGATG CTTTAGATGG CAAGCGTATG |
| | ATAACGATTG |
| 2951 | CCGTAAACAG CACAAGTGGG GGATTAAAAA TCTCAGGGGA |
| | TCTGAAATTC |
| 3001 | CATAACAATG AAGGAAGTTT CTATGACAAT CCTGGGTTGA |
| | AAGCAAACTT |
| 3051 | AAATCTTCCT TTCTTAGATC TTTCTTCTAC TTCAGGAACT |
| | GTAAATTTAG |
| 3101 | ACGACTTCAA TCCGATTCCT TCTAGCATGG CTGCTCCGGA |
| | TTATGGGTAT |
| 3151 | CAAGGGAGTT GGACTCTGGT TCCTAAAGTA GGAGCTGGAG |
| | GGAAGGTGAC |
| 3201 | TTTGGTCGCG GAATGGCAAG CGTTAGGATA CACTCCTAAA |
| | CCAGAGCTTC |
| 3251 | GTGCGACTTT AGTTCCTAAT AGCCTTTGGA ATGCTTATGT |
| | AAACATCCAT |
| 3301 | TCTATACAGC AGGAGATCGC CACTGCGATG TCGGACGCTC |
| | CCTCACATCC |
| 3351 | AGGGATTTGG ATTGGAGGTA TTGGCAACGC CTTCCATCAA |
| | GACAAGCAAA |
| 3401 | AGGAAAATGC AGGATTCCGT TTGATTTCCA GAGGTTATAT |
| | TGTTGGTGGC |
| 3451 | AGCATGACCA CCCCTCAAGA ATATACCTTT GCTGTTGCAT |
| | TCAGCCAACT |
| 3501 | CTTTGGCAAA TCTAAGGATT ACGTAGTCTC GGATATTAAA |
| | TCTCAAGTCT |
| 3551 | ATGCAGGATC TCTCTGTGCT CAGAGCTCTT ATGTCATTCC |
| | CCTGCATAGC |
| 3601 | TCATTACGTC GCCACGTCCT CTCTAAGGTC CTTCCAGAGC |
| | TCCCAGGAGA |
| 3651 | AACTCCCCTT GTTCTCCATG GTCAAGTTTC CTATGGAAGA |
| | AACCACCATA |
| 3701 | ATATGACGAC AAAGCTTGCG AACAACACAC AAGGGAAATC |
| | AGACTGGGAC |
| 3751 | AGCCATAGCT TCGCTGTTGA AGTCGGTGGT TCTCTTCCTG |
| | TAGATCTAAA |
| 3801 | CTACAGATAC CTTACCAGCT ACTCTCCCTA TGTGAAACTC |
| | CAAGTTGTGA |
| 3851 | GTGTAAATCA AAAAGGATTC CAAGAGGTTG CTGCTGATCC |
| | ACGTATCTTT |
| 3901 | GACGCTAGCC ATCTGGTCAA CGTGTCTATC CCTATGGGAC |
| | TCACCTTCAA |
| 3951 | ACACGAATCA GCAAAGCCCC CCAGTGCTTT GCTTCTTACT |
| | TTAGGTTACG |
| 4001 | CTGTAGATGC TTACCGGGAT CACCCTCACT GCCTGACCTC |
| | CTTAACAAAT |
| 4051 | GGCACCTCGT GGTCTACGTT TGCTACAAAC TTATCACGAC |
| | AAGCTTTCTT |
| 4101 | TGCTGAGGCT TCTGGACATC TGAAGTTACT TCATGGTCTT |
| | GACTGCTTCG |
| 4151 | CTTCTGGAAG TTGTGAACTG CGCAGCTCCT CAAGAAGCTA |
| | TAATGCAAAC |
| 4201 | TGTGGAACTC GTTATTCTTT CTAA |

The PSORT algorithm predicts an outer membrane location (0.915).

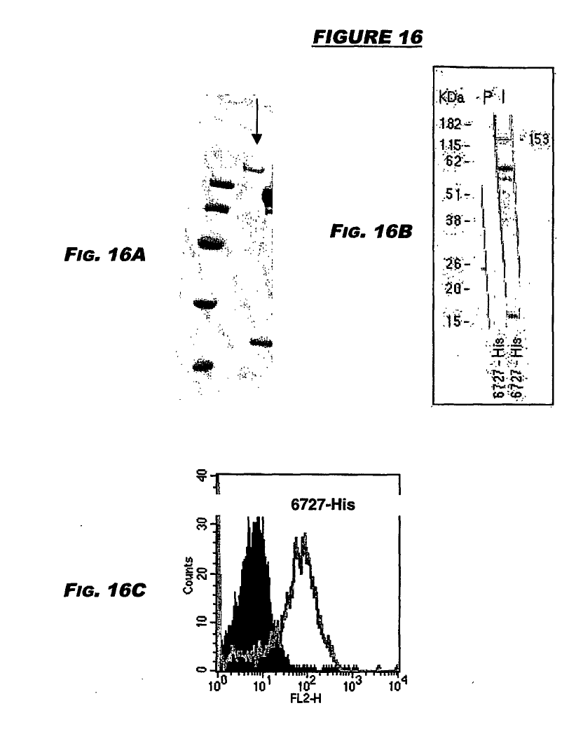

The protein was expressed in E. coli and purified as a his-tag product, as shown in FIG. 16A. The recombinant protein was used to immunise mice, whose sera were used in a Western blot (FIG. 16B) and for FACS analysis (FIG. 16C). A GST-fusion protein was also expressed.

The cp6727 protein was also identified in the 2D-PAGE experiment (Cpn0444).

These experiments show that cp6727 is a surface-exposed and immunoaccessible protein, and that it is a useful immunogen. These properties are not evident from the sequence alone.

### Example 17

The following C. pneumoniae protein (PID 4376731) was expressed <SEQ ID 33; cp6731>:

| | |
|---|---|
| 1 | MKSSLHWFLI SSSLALPLSL NFSAFAAVVE INLGPTNSFS |
| | GPGTYTPPAQ |
| 51 | TTNADGTIYN LTGDVSITNA GSPTALTASC FKETTGNLSF |
| | QGHGYQFLLQ |
| 101 | NIDAGANCTF TNTAANKLLS FSGFSYLSLI QTTNATTGTG |
| | AIKSTGACSI |
| 151 | QSNYSCYFGQ NFSNDNGGAL QGSSISLSLN PNLTFAKNKA |
| | TQKGGALYST |
| 201 | GGITINNTLN SASFSENTAA NNGGAIYTEA SSFISSNKAI |
| | SFINNSVTAT |
| 251 | SATGGAIYCS STSAPKPVLT LSDNGELNFI GNTAITSGGA |
| | IYTDNLVLSS |
| 301 | GGPTLFKNNS AIDTAAPLGG AIAIADSGSL SLSALGGDIT |
| | FEGNTVVKGA |
| 351 | SSSQTTTRNS INIGNTNAKI VQLRASQGNT IYFYDPITTS |
| | ITAALSDALN |
| 401 | LNGPDLAGNP AYQGTIVFSG EKLSEAEAAE ADNLKSTIQQ |
| | PLTLAGGQLS |
| 451 | LKSGVTLVAK SFSQSPGSTL LMDAGITLET ADGITINNLV |
| | LNVDSLKETK |
| 501 | KATLKATQAS QTVTLSGSLS LVDPSGNVYE DVSWNNPQVF |
| | SCLTLTADDP |
| 551 | ANIHITDLAA DPLEKNPIHW GYQGNWALSW QEDTATKSKA |
| | ATLTWTKTGY |
| 601 | NPNPERRGTL VANTLWGSFV DVRSIQQLVA TKVRQSQETR |
| | GIWCEGISNF |
| 651 | FHKDSTKINK GFRHISAGYV VGATTTLASD NLITAAFCQL |
| | FGKDRDHFIN |
| 701 | KNRASAYAAS LHLQHLATLS SPSLLRYLPG SESEQPVLFD |
| | AQISYIYSKN |
| 751 | TMKTYYTQAP KGESSWYNDG CALELASSLP HTALSHEGLF |
| | HAYFPFIKVE |
| 801 | ASYIHQDSFK ERNTTLVRSF DSGDLINVSV PIGITFERFS |
| | RNERASYEAT |
| 851 | VIYVADVYRK NPDCTTALLI NNTSWKTTGT NLSRQAGIGR |
| | AGIFYAFSPN |
| 901 | LEVTSNLSME IRGSSRSYNA DLGGKFQF* |

A predicted signal peptide is highlighted.

The cp6731 nucleotide sequence <SEQ ID 34> is:

| | |
|---|---|
| 1 | ATGAAATCCT CTCTTCATTG GTTTTTAATC TCGTCATCTT |
| | TAGCACTTCC |
| 51 | CTTGTCACTA AATTTCTCTG CGTTTGCTGC TGTTGTTGAA |
| | ATCAATCTAG |
| 101 | GACCTACCAA TAGCTTCTCT GGACCAGGAA CCTACACTCC |
| | TCCAGCCCAA |
| 151 | ACAACAAATG CAGATGGAAC TATCTATAAT CTAACAGGGG |
| | ATGTCTCAAT |
| 201 | CACCAATGCA GGATCTCCGA CAGCTCTAAC CGCTTCCTGC |
| | TTTAAAGAAA |
| 251 | CTACTGGGAA TCTTTCTTTC CAAGGCCACG GCTACCAATT |
| | TCTCCTACAA |
| 301 | AATATCGATG CGGGAGCGAA CTGTACCTTT ACCAATACAG |
| | CTGCAAATAA |
| 351 | GCTTCTCTCC TTTTCAGGAT TCTCCTATTT GTCACTAATA |
| | CAAACCACGA |
| 401 | ATGCTACCAC AGGAACAGGA GCCATCAAGT CCACAGGAGC |
| | TTGTTCTATT |
| 451 | CAGTCGAACT ATAGTTGCTA CTTTGGCCAA AACTTTTCTA |
| | ATGACAATGG |
| 501 | AGGCGCCCTC CAAGGCAGCT CTATCAGTCT ATCGCTAAAC |
| | CCCAACCTAA |
| 551 | CGTTTGCCAA AAACAAAGCA ACGCAAAAAG GGGGTGCCCT |
| | CTATTCCACG |
| 601 | GGAGGGATTA CAATTAACAA TACGTTAAAC TCAGCATCAT |
| | TTTCTGAAAA |
| 651 | TACCGCGGCG AACAATGGCG GAGCCATTTA CACGGAAGCT |
| | AGCAGTTTTA |
| 701 | TTAGCAGCAA CAAAGCAATT AGCTTTATAA ACAATAGTGT |
| | GACCGCAACC |
| 751 | TCAGCTACAG GGGGAGCCAT TTACTGTAGT AGTACATCAG |
| | CCCCCAAACC |
| 801 | AGTCTTAACT CTATCAGACA ACGGGGAACT GAACTTTATA |
| | GGAAATACAG |
| 851 | CAATTACTAG TGGTGGGGCG ATTTATACTG ACAATCTAGT |
| | TCTTTCTTCT |
| 901 | GGAGGACCTA CGCTTTTTAA AAACAACTCT GCTATAGATA |
| | CTGCAGCTCC |
| 951 | CTTAGGAGGA GCAATTGCGA TTGCTGACTC TGGATCTTTG |
| | AGTCTTTCGG |
| 1001 | CTCTTGGTGG AGACATCACT TTTGAAGGAA ACACAGTAGT |
| | CAAAGGAGCT |
| 1051 | TCTTCGAGTC AGACCACTAC CAGAAATTCT ATTAACATCG |
| | GAAACACCAA |
| 1101 | TGCTAAGATT GTACAGCTGC GAGCCTCTCA AGGCAATACT |
| | ATCTACTTCT |
| 1151 | ATGATCCTAT AACAACTAGC ATCACTGCAG CTCTCTCAGA |
| | TGCTCTAAAC |
| 1201 | TTAAATGGTC CTGACCTTGC AGGGAATCCT GCATATCAAG |
| | GAACCATCGT |
| 1251 | ATTTTCTGGA GAGAAGCTCT CGGAAGCAGA AGCTGCAGAA |
| | GCTGATAATC |
| 1301 | TCAAATCTAC AATTCAGCAA CCTCTAACTC TTGCGGGAGG |
| | GCAACTCTCT |
| 1351 | CTTAAATCAG GAGTCACTCT AGTTGCTAAG TCCTTTTCGC |
| | AATCTCCGGG |
| 1401 | CTCTACCCTC CTCATGGATG CAGGGACCAC ATTAGAAACC |
| | GCTGATGGGA |
| 1451 | TCACTATCAA TAATCTTGTT CTCAATGTAG ATTCCTTAAA |
| | AGAGACCAAG |
| 1501 | AAGGCTACGC TAAAAGCAAC ACAAGCAAGT CAGACAGTCA |
| | CTTTATCTGG |
| 1551 | ATCGCTCTCT CTTGTAGATC CTTCTGGAAA TGTCTACGAA |
| | GATGTCTCTT |
| 1601 | GGAATAACCC TCAAGTCTTT TCTTGTCTCA CTCTTACTGC |
| | TGACGACCCC |
| 1651 | GCGAATATTC ACATCACAGA CTTAGCTGCT GATCCCCTAG |
| | AAAAAAATCC |
| 1701 | TATCCATTGG GGATACCAAG GGAATTGGGC ATTATCTTGG |
| | CAAGAGGATA |
| 1751 | CTGCGACTAA ATCCAAAGCA GCGACTCTTA CCTGGACAAA |
| | AACAGGATAC |
| 1801 | AATCCGAATC CTGAGCGTCG TGGAACCTTA GTTGCTAACA |
| | CGCTATGGGG |
| 1851 | ATCCTTTGTT GATGTGCGCT CCATACAACA GCTTGTAGCC |
| | ACTAAAGTAC |
| 1901 | GCCAATCTCA AGAAACTCGC GGCATCTGGT GTGAAGGGAT |
| | CTCGAACTTC |
| 1951 | TTCCATAAAG ATAGCACGAA GATAAATAAA GGTTTTCGCC |
| | ACATAAGTGC |
| 2001 | AGGTTATGTT GTAGGAGCGA CTACAACATT AGCTTCTGAT |
| | AATCTTATCA |
| 2051 | CTGCAGCCTT CTGCCAATTA TTCGGGAAAG ATAGAGATCA |
| | CTTTATAAAT |
| 2101 | AAAAATAGAG CTTCTGCCTA TGCAGCTTCT CTCCATCTCC |
| | AGCATCTAGC |
| 2151 | GACCTTGTCT TCTCCAAGCT TGTTACGCTA CCTTCCTGGA |
| | TCTGAAAGTG |
| 2201 | AGCAGCCTGT CCTCTTCGAT GCTCAGATCA GCTATATCTA |
| | TAGTAAAAAT |
| 2251 | ACTATGAAAA CCCATTACAC CCAAGCACCA AAGGGAGAGA |
| | GCTCGTGGTA |
| 2301 | TAATGACGGT TGCGCTCTGG AACTTGCGAG CTCCCTACCA |
| | CACACTGCTT |
| 2351 | TAAGCCATGA GGGTCTCTTC CACGCGTATT TTCCTTTCAT |
| | CAAAGTAGAA |
| 2401 | GCTTCGTACA TACACCAAGA TAGCTTCAAA GAACGTAATA |
| | CTACCTTGGT |
| 2451 | ACGATCTTTC GATAGCGGTG ATTTAATTAA CGTCTCTGTG |
| | CCTATTGGAA |
| 2501 | TTACCTTCGA GAGATTCTCG AGAAACGAGC GTGCGTCTTA |
| | CGAAGCTACT |
| 2551 | GTCATCTACG TTGCCGATGT CTATCGTAAG AATCCTGACT |
| | GCACGACAGC |
| 2601 | TCTCCTAATC AACAATACCT CGTGGAAAAC TACAGGAACG |
| | AATCTCTCAA |
| 2651 | GACAAGCTGG TATCGGAAGA GCAGGGATCT TTTATGCCTT |
| | CTCTCCAAAT |
| 2701 | CTTGAGGTCA CAAGTAACCT ATCTATGGAA ATTCGTGGAT |
| | CTTCACGCAG |
| 2751 | CTACAATGCA GATCTTGGAG GTAAGTTCCA GTTCTAA |

The PSORT algorithm predicts an outer membrane location (0.926).

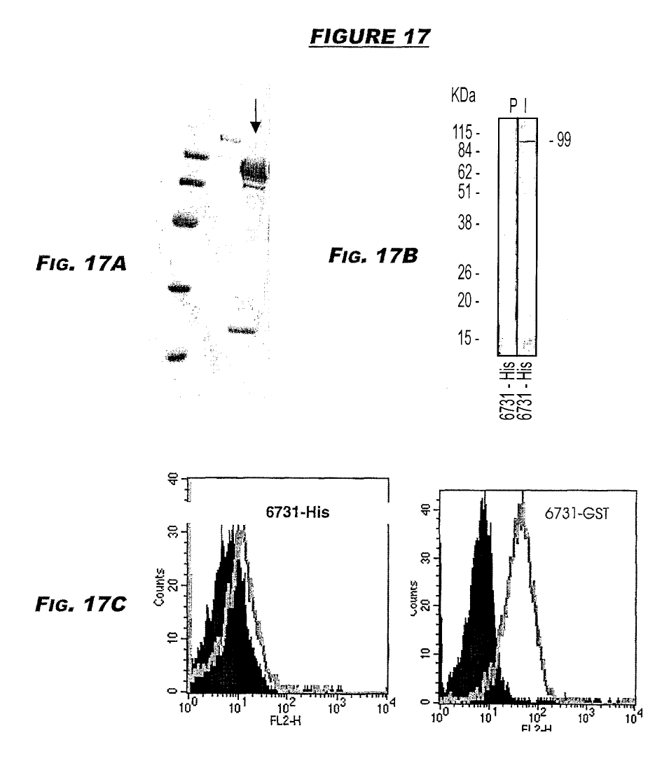

The protein was expressed in E. coli and purified as a his-tag product, as shown in FIG. 17A. A GST-fusion protein was also expressed. The recombinant proteins were used to immunise mice, whose sera were used in a Western blot (FIG. 17B; his-tag) and for FACS analysis (FIG. 17C; his-tag and GST-fusion).

The GST-fusion protein also showed good cross-reactivity with human sera, including sera from patients with pneumonitis. Less cross-reactivity was seen with the his-fusion.

These experiments show that cp6731 is a surface-exposed and immunoaccessible protein, and that it is a useful immunogen. These properties are not evident from the sequence alone.

### Example 18

The following C. pneumoniae protein (PID 4376737) was expressed <SEQ ID 35; cp6737>:

| | |
|---|---|
| 1 | MPLSFKSSSF CLLACLCSAS CAFAETRLGG NFVPPITNQG |
| | EEILLTSDFV |
| 51 | CSNFLGASFS SSFINSSSNL SLLGKGLSLT FTSCQAPTNS |
| | NYALLSAAET |
| 101 | LTFKNFSSIN FTGNQSTGLG GLIYGKDIVF QSIKDLIFTT |
| | NRVAYSPASV |
| 151 | TTSATPAITT VTTGASALQP TDSLTVENIS QSIKFFGNLA |
| | NFGSAISSSP |
| 201 | TAVVKFINNT ATMSFSHNFT SSGGGVIYGG SSLLFENNSG |
| | CIIFTANSCV |
| 251 | NSLKGVTPSS GTYALGSGGA ICIPTGTFEL KNNQGKCTFS |
| | YNGTPNDAGA |
| 301 | IYAFTCNIVG NQGALLLDSN TAARNGGAIC AKVLNIQGRG |
| | PIEFSRNRAE |
| 351 | KGGAIFIGPS VGDPAKQTST LTILASEGDI AFQGNMLNTK |
| | PGIRNAITVE |
| 401 | AGGEIVSLSA QGGSRLVFYD PITHSLPTTS PSNKDITINA |
| | NGASGSVVFT |
| 451 | SKGLSSTELL LPANTTTILL GTVKIASGEL KITDNAVVNV |
| | LGFATQGSGQ |
| 501 | LTLGSGGTLG LATPTGAPAA VDFTIGKLAF DPFSFLKRDF |
| | VSASVNAGTK |
| 551 | NVTLTGALVL DEHDVTDLYD MVSLQTPVAI PIAVFKGATV |
| | TKTGFPDGEI |
| 601 | ATPSHYGYQG KWSYTWSRPL LIPAPDGGFP GGPSPSANTL |
| | YAVWNSDTLV |
| 651 | RSTYILDPER YGEIVSNSLW ISFLGNQAFS DILQDVLLID |
| | HPGLSITAKA |
| 701 | LGAYVEHTPR QGHEGFSGRY GSYQAALSMN YTDHTTLGLS |
| | FGQLYGKTNA |
| 751 | NPYDSRCSEQ MYLLSFFGQF PIVTQKSEAL ISWKAAYGYS |
| | KNHLNTTYLR |
| 801 | PDKAPKSQGQ WHNNSYYVLI SAEHPFLNWC LLTRPLAQAW |
| | DLSGFISAEF |
| 851 | LGGWQSKFTE TGDLQRSFSR GKGYNVSLPI GCSSQWFTPF |
| | KKAPSTLTIK |
| 901 | LAYKPDIYRV NPHNIVTVVS NQESTSISGA NLRRHGLFVQ |
| | IHDVVDLTED |
| 951 | TQAFLNYTFD GKNGFTNHRV STGLKSTF* |

A predicted signal peptide is highlighted.

The cp6737 nucleotide sequence <SEQ ID 36> is:

| | |
|---|---|
| 1 | ATGCCTCTTT CTTTCAAATC TTCATCTTTT TGTCTACTTG |
| | CCTGTTTATG |
| 51 | TAGTGCAAGT TGCGCGTTTG CTGAGACTAG ACTCGGAGGG |
| | AACTTTGTTC |
| 101 | CTCCAATTAC GAATCAGGGT GAAGAGATCT TACTCACTTC |
| | AGATTTTGTT |
| 151 | TGTTCAAACT TCTTGGGGGC GAGTTTTTCA AGTTCCTTTA |
| | TCAATAGTTC |
| 201 | CAGCAATCTC TCCTTATTAG GGAAGGGCCT TTCCTTAACG |
| | TTTACCTCTT |
| 251 | GTCAAGCTCC TACAAATAGT AACTATGCGC TACTTTCTGC |
| | CGCAGAGACT |
| 301 | CTGACCTTCA AGAATTTTTC TTCTATAAAC TTTACAGGGA |
| | ACCAATCGAC |
| 351 | AGGACTTGGC GGCCTCATCT ACGGAAAAGA TATTGTTTTC |
| | CAATCTATCA |
| 401 | AAGATTTGAT CTTCACTACG AACCGTGTTG CCTATTCTCC |
| | AGCATCTGTA |
| 451 | ACTACGTCGG CAACTCCCGC AATCACTACA GTAACTACAG |
| | GAGCCTCTGC |
| 501 | TCTCCAACCT ACAGACTCAC TCACTGTCGA AAACATATCC |
| | CAATCGATCA |
| 551 | AGTTTTTTGG GAACCTTGCC AACTTCGGCT CTGCAATTAG |
| | CAGTTCTCCC |
| 601 | ACGGCAGTCG TTAAATTCAT CAATAACACC GCTACCATGA |
| | GCTTCTCCCA |
| 651 | TAACTTTACT TCGTCAGGAG GCGGCGTGAT TTATGGAGGA |
| | AGCTCTCTCC |
| 701 | TTTTTGAAAA CAATTCTGGA TGCATCATCT TCACCGCCAA |
| | CTCCTGTGTG |
| 751 | AACAGCTTAA AAGGCGTCAC CCCTTCATCA GGAACCTATG |
| | CTTTAGGAAG |
| 801 | TGGCGGAGCC ATCTGCATCC CTACGGGAAC TTTCGAATTA |
| | AAAAACAATC |
| 851 | AGGGGAAGTG CACCTTCTCT TATAATGGTA CACCAAATGA |
| | TGCGGGTGCG |
| 901 | ATCTACGCCG AAACCTGCAA CATCGTAGGG AACCAGGGTG |
| | CCTTGCTCCT |
| 951 | AGATAGCAAC ACTGCAGCGA GAAATGGCGG AGCCATCTGT |
| | GCTAAAGTGC |
| 1001 | TCAATATTCA AGGACGCGGT CCTATTGAAT TCTCTAGAAA |
| | CCGCGCGGAG |
| 1051 | AAGGGTGGAG CTATTTTCAT AGGCCCCTCT GTTGGAGACC |
| | CTGCGAAGCA |
| 1101 | AACATCGACA CTTACGATTT TGGCTTCCGA AGGTGATATT |
| | GCGTTCCAAG |
| 1151 | GAAACATGCT CAATACAAAA CCTGGAATCC GCAATGCCAT |
| | CACTGTAGAA |
| 1201 | GCAGGGGGAG AGATTGTGTC TCTATCTGCA CAAGGAGGCT |
| | CACGTCTTGT |
| 1251 | ATTTTATGAT CCCATTACAC ATAGCCTCCC AACCACAAGT |
| | CCGTCTAATA |
| 1301 | AAGACATTAC AATCAACGCT AATGGCGCTT CAGGATCTGT |
| | AGTCTTTACA |
| 1351 | AGTAAGGGAC TCTCCTCTAC AGAACTCCTG TTGCCTGCCA |
| | AGACGACAAC |
| 1401 | TATACTTCTA GGAACAGTCA AGATCGCTAG TGGAGAACTG |
| | AAGATTACTG |
| 1451 | ACAATGCGGT TGTCAATGTT CTTGGCTTCG CTACTCAGGG |
| | CTCAGGTCAG |
| 1501 | CTTACCCTGG GGTCTGGAGG AACCTTAGGG CTGGCAACAC |
| | CCACGGGAGC |
| 1551 | ACCTGCCGCT GTAGACTTTA CGATTGGAAA GTTAGCATTC |
| | GATCCTTTTT |
| 1601 | CCTTCCTAAA AAGAGATTTT GTTTCAGCAT CAGTAAATGC |
| | AGGCACAAAA |
| 1651 | AACGTCACTT TAACAGGAGC TCTGGTTCTT GATGAACATG |
| | ACGTTACAGA |
| 1701 | TCTTTATGAT ATGGTGTCAT TACAAACTCC AGTAGCAATT |
| | CCTATCGCTG |
| 1751 | TTTTCAAAGG AGCAACCGTT ACTAAGACAG GATTTCCTGA |
| | TGGGGAGATT |
| 1801 | GCGACTCCAA GCCACTACGG CTACCAAGGA AAGTGGTCCT |
| | ACACATGGTC |
| 1851 | CCGTCCCCTG TTAATTCCAG CTCCTGATGG AGGATTTCCT |
| | GGAGGTCCCT |
| 1901 | CTCCTAGCGC AAATACTCTC TATGCTGTAT GGAATTCAGA |
| | CACTCTCGTG |
| 1951 | CGTTCTACCT ATATCTTAGA TCCCGAGCGT TACGGAGAAA |
| | TTGTCAGCAA |
| 2001 | CAGCTTATGG ATTTCCTTCT TAGGAAATCA GGCATTCTCT |
| | GATATTCTCC |
| 2051 | AAGATGTTCT TTTGATAGAT CATCCCGGGT TCTCCATAAC |
| | CGCGAAAGCT |
| 2101 | TTAGGAGCCT ATGTCGAACA CACACCAAGA CAAGGACATG |
| | AGGGCTTTTC |
| 2151 | AGGTCGCTAT GGAGGCTACC AAGCTGCGCT ATCTATGAAC |
| | TACACGGACC |
| 2201 | ACACTACGTT AGGACTTTCT TTCGGGCAGC TTTATGGAAA |
| | AACTAACGCC |
| 2251 | AACCCCTACG ATTCACGTTG CTCAGAACAA ATGTATTTAC |
| | TCTCGTTCTT |
| 2301 | TGGTCAATTC CCTATCGTGA CTCAAAAGAG CGAGGCCTTA |
| | ATTTCCTGGA |
| 2351 | AAGCAGCTTA TGGTTATTCC AAAAATCACC TAAATACCAC |
| | CTACCTCAGA |
| 2401 | CCTGACAAAG CTCCAAAATC TCAAGGGCAA TGGCATAACA |
| | ATAGTTACTA |
| 2451 | TGTTCTTATT TCTGCAGAAC ATCCTTTCCT AAACTGGTGT |
| | CTTCTTACAA |
| 2501 | GACCTCTGGC TCAAGCTTGG GATCTTTCAG GTTTTATTTC |
| | CGCAGAATTC |
| 2551 | CTAGGTGGTT GGCAAAGTAA GTTCACAGAA ACTGGAGATC |
| | TGCAACGTAG |
| 2601 | CTTTAGTAGA GGTAAAGGGT ACAATGTTTC CCTACCGATA |
| | GGATGTTCTT |
| 2651 | CTCAATGGTT CACACCATTT AAGAAGGCTC CTTCTACACT |
| | GACCATCAAA |
| 2701 | CTTGCCTACA AGCCTGATAT CTATCGTGTC AACCCTCACA |
| | ATATTGTGAC |
| 2751 | TGTCGTCTCA AACCAAGAGA GCACTTCGAT CTCAGGAGCA |
| | AATCTACGCC |
| 2801 | GCCACGGTTT GTTTGTACAA ATCCATGATG TAGTAGATCT |
| | CACCGAGGAC |
| 2851 | ACTCAGGCCT TTCTAAACTA TACCTTTGAC GGGAAAAATG |
| | GATTTACAAA |
| 2901 | CCACCGAGTG TCTACAGGAC TAAAATCCAC ATTTTAA |

The PSORT algorithm predicts an outer membrane location (0.940).

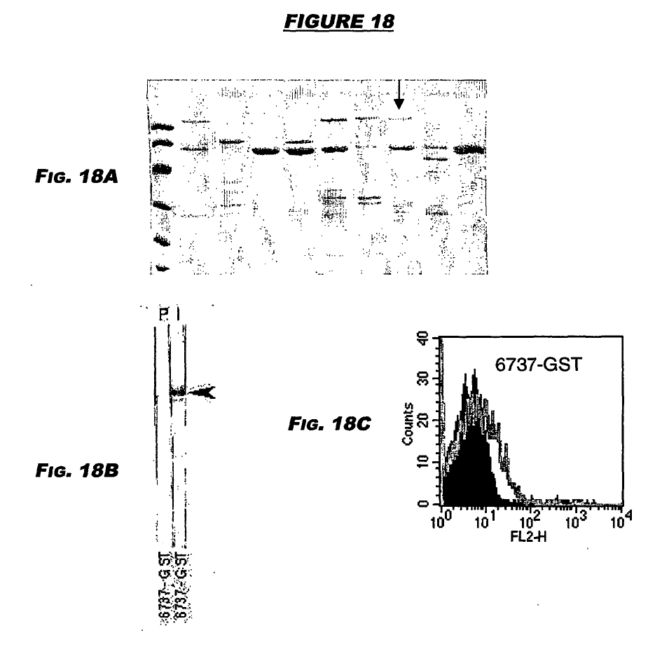

The protein was expressed in E. coli and purified as a GST-fusion product, as shown in FIG. 18A. The recombinant protein was used to immunise mice, whose sera were used in an immunoblot analysis blot (FIG. 18B) and for FACS analysis (FIG. 18C). A his-tagged protein was also expressed.

The cp6737 protein was also identified in the 2D-PAGE experiment (Cpn0454) and showed good cross-reactivity with human sera, including sera from patients with pneumonitis.

These experiments show that cp6737 is a surface-exposed and immunoaccessible protein, and that it is a useful immunogen. These properties are not evident from the sequence alone.

### Example 19

The following C. pneumoniae protein (PID 4377090) was expressed <SEQ ID 37; cp7090>:

| | |
|---|---|
| 1 | MNIHSLWKLC TLLALLALPA CSLSRNYGWE DSCNTCHHTR |
| | RKKPSSFGFV |
| 51 | PLYTEEDFNP NFTFGEYDSK EEKQYKSSQV AAFRNITFAT |
| | DSYTIKGEEN |
| 101 | LAILTNLVHY MKKNPKATLY IEGHTDERGA ASYNLALGAR |
| | RANAIKEHLR |
| 151 | KQGISADRLS TISYGKEHPL NSGHNELAWQ QNRRTEFKIH AR* |

A predicted signal peptide is highlighted.

The cp7090 nucleotide sequence <SEQ ID 38> is:

| | |
|---|---|
| 1 | ATGAATATAC ATTCCCTATG GAAACTTTGT ACTTTATTGG |
| | CTTTACTTGC |
| 51 | ATTGCCAGCA TGTAGCCTTT CCCCTAATTA TGGCTGGGAG |
| | GATTCCTGTA |
| 101 | ATACATGCCA TCATACAAGA CGAAAAAAGC CTTCTTCTTT |
| | TGGCTTTGTT |
| 151 | CCTCTCTATA CCGAAGAGGA CTTTAACCCT AATTTTACCT |
| | TCGGTGAGTA |
| 201 | TGATTCCAAA GAAGAAAAAC AATACAAGTC AAGCCAAGTT |
| | GCAGCATTTC |
| 251 | GTAATATCAC CTTTGCTACA GACAGCTATA CAATTAAAGG |
| | TGAAGAGAAC |
| 301 | CTTGCGATTC TCACGAACTT GGTTCACTAC ATGAAGAAAA |
| | ACCCGAAAGC |
| 351 | TACACTGTAC ATTGAAGGGC ATACTGACGA GCGTGGAGCT |
| | GCATCCTATA |
| 401 | ACCTTGCTTT AGGAGCACGA CGAGCCAATG CGATTAAAGA |
| | GCATCTCCGA |
| 451 | AAGCAGGGAA TCTCTGCAGA TCGTCTATCT ACTATTTCCT |
| | ACGGAAAAGA |
| 501 | ACATCCTTTA AATTCGGGAC ACAACGAACT AGCATGGCAA |
| | CAAAATCGCC |
| 551 | GTACAGAGTT TAAGATTCAT GCACGCTAA |

The PSORT algorithm predicts an outer membrane location (0.790).

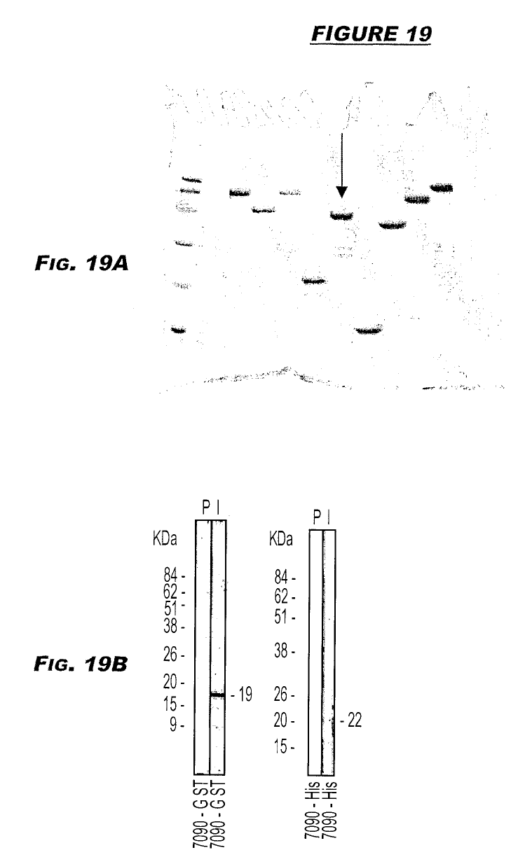

The protein was expressed in E. coli and purified as a GST-fusion product, as shown in FIG. 19A. A his-tagged protein was also expressed. The recombinant proteins were used to immunise mice, whose sera were used in a Western blot (FIG. 19B) and for FACS analysis.

These experiments show that cp7090 is useful immunogen. These properties are not evident from the sequence alone.

### Example 20

The following C. pneumoniae protein (PID 4377091) was expressed <SEQ ID 39; cp7091>:

| | |
|---|---|
| 1 | MLRQLCFQVF FFCFASLVYA EELEVVVRSE HITLPIEVSC |
| | QTDTKDPKIQ |
| 51 | KYLSSLTEIF CKDIALGDCL QPTAASKESS SPLAISLRLH |
| | VPQLSVVLLQ |
| 101 | SSKTPQTLCS FTISQNLSVD RQKIHHAADT VHYALTGIPG |
| | ISAGKIVFAL |
| 151 | SSLGKDQKLK QGELWTTDYD GKNLAPLTTE CSLSITPKWV |
| | GVGSNFPYLY |
| 201 | VSYKYGVPKI FLGSLENTEG KKVLPLKGNQ LMPTFSPRKK |
| | LLAFVADTYG |
| 251 | NPDLFIQPFS LTSGPMGRPR RLLNENFGTQ GNPSFNPEGS |
| | QLVFISNKDG |
| 301 | RPRLYIMSLD PEPQAPRLLT KKYRNSSCPA WSPDGKKIAF |
| | CSVIKGVRQI |
| 351 | CIYDLSSGED YQLTTSPTNK ESPSWAIDSR HLVFSAGNAE |
| | ESELYLISLV |
| 401 | TKKTNKIAIG VGEKRFPSWG AFPQQPIKRT L* |

A predicted signal peptide is highlighted.

The cp7091 nucleotide sequence <SEQ ID 40> is:

| | |
|---|---|
| 1 | ATGTTACGGC AACTATGCTT CCAAGTTTTT TTCTTTTGCT |
| | TCGCATCGCT |
| 51 | AGTCTATGCT GAAGAATTAG AAGTTGTTGT CCGTTCCGAA |
| | CATATCACGC |
| 101 | TCCCTATTGA GGTCTCTTGC CAGACCGATA CGAAAGATCC |
| | AAAAATACAG |
| 151 | AAATACCTCA GCTCGCTAAC GGAGATATTT TCGAAGGACA |
| | TTGCCCTAGG |
| 201 | AGATTGTCTA CAACCCACAG CGGCTTCTAA AGAATCGTCA |
| | TCTCCTTTAG |
| 251 | CAATATCTTT ACGGTTGCAT GTACCTCAGC TATCTGTAGT |
| | GCTTTTACAG |
| 301 | TCTTCAAAAA CTCCTCAAAC CTTATGTTCT TTTACTATTT |
| | CTCAAAATCT |
| 351 | TTCTGTAGAT CGTCAAAAAA TCCATCACGC TGCTGATACA |
| | GTTCATTACG |
| 401 | CCCTCACAGG GATTCCTGGA ATCAGTGCTG GGAAAATTGT |
| | TTTTGCTCTA |
| 451 | AGTTCTTTAG GAAAAGATCA AAAGCTCAAG CAAGGAGAAT |
| | TATGGACTAC |
| 501 | AGATTACGAT GGGAAAAACC TCGCCCCTTT AACCACAGAA |
| | TGTTCGCTCT |
| 551 | CTATAACTCC AAAATGGGTG GGTGTGGGAT CAAATTTTCC |
| | CTATCTCTAT |
| 601 | GTTTCGTATA AGTATGGTGT GCCTAAAATT TTTCTTGGTT |
| | CCCTAGAGAA |
| 651 | CACTGAAGGT AAAAAAGTCC TTCCGTTAAA AGGCAACCAA |
| | CTCATGCCTA |
| 701 | CGTTTTCTCC AAGAAAAAAG CTTTTAGCTT TCGTTGCTGA |
| | TACGTATGGA |
| 751 | AATCCTGATT TATTTATTCA ACCGTTCTCA CTAACTTCAG |
| | GACCTATGGG |
| 801 | TCGCCCACGT CGCCTCCTTA ATGAGAATTT CGGGACTCAA |
| | GGGAATCCCT |
| 851 | CCTTCAACCC TGAAGGATCC CAGCTTGTCT TTATATCGAA |
| | CAAAGACGGC |
| 901 | CGTCCGCGTC TTTATATTAT GTCCCTCGAT CCTGAACCCC |
| | AAGCACCTCG |
| 951 | CTTGCTGACA AAAAAATACA GAAATAGCAG TTGCCCTGCA |
| | TGGTCTCCAG |
| 1001 | ATGGTAAAAA AATAGCCTTC TGCTCTGTAA TTAAAGGGGT |
| | GCGACAAATT |
| 1051 | TGTATTTACG ATCTCTCCTC TGGAGAGGAT TACCAACTCA |
| | CTACGTCTCC |
| 1101 | CACAAATAAA GAGAGTCCTT CTTGGGCTAT AGACAGCCGT |
| | CATCTTGTCT |
| 1151 | TTAGTGCGGG GAATGCTGAA GAATCAGAGT TATATTTAAT |
| | CAGTCTAGTC |
| 1201 | ACCAAAAAAA CTAACAAAAT TGCTATAGGA GTAGGAGAAA |
| | AACGGTTCCC |
| 1251 | CTCCTGGGGT GCTTTCCCTC AGCAACCGAT AAAGAGAACA |
| | CTATGA |

The PSORT algorithm predicts an inner membrane location (0.109).

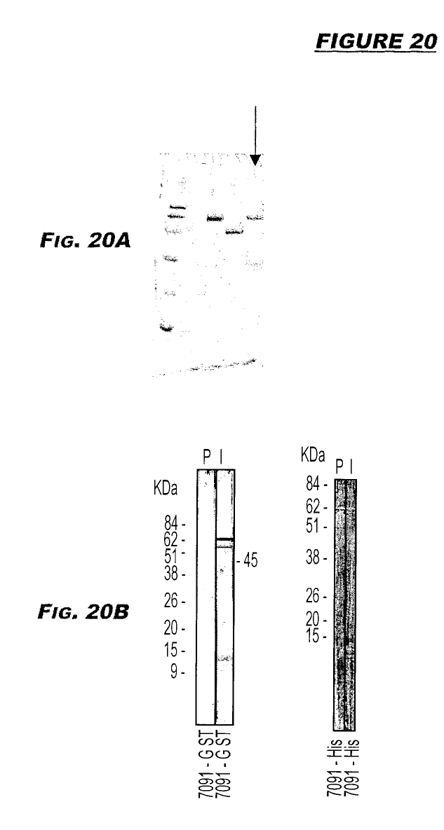

The protein was expressed in E. coli and purified as a GST-fusion product, as shown in FIG. 20A. A his-tagged protein was also expressed. The recombinant proteins were used to immunise mice, whose sera were used in a Western blot (FIG. 20B) and for FACS analysis.

These experiments show that cp7091 is a useful immunogen. These properties are not evident from the sequence alone.

### Example 21

The following C. pneumoniae protein (PID 4376260) was expressed <SEQ ID 41; cp6260>:

| | |
|---|---|
| 1 | MRFSLCGFPL VFSFTLLSVF DTSLSATTIS LTPEDSFEGD |
| | SQNAERSYNV |
| 51 | QAGDVYSLTG DVSISNVDNS ALNKACFNVT SGSVTFAGNH |
| | HGLYFNNISS |
| 101 | GTTKEGAVLC CQDPQATARF SGFSTLSFIQ SPGDIKEQGC |
| | LYSKNALMLL |
| 151 | NNYVVRFEQN QSKTKGGAIS GANVTIVGNY DSVSFYQNAA |
| | TFGGAIHSSG |
| 201 | PLQIAVNQAE IRFAQNTAKN GSGGALYSDG DIDIDQNAYV |
| | LFRENEALTT |
| 251 | AIGKGGAVCC IPTSGSSTPV PIVTFSDNKQ LVFERNHSIM |
| | GGGAIYARKL |
| 301 | SISSGGPTLF INNISYANSQ NLGGAIAIDT GGEISLSAEK |
| | GTITFQGNRT |
| 351 | SLPFLNGIHL LQNAKFLKLQ ARNGYSIEFY DPITSEADGS |
| | TQLNINGDPK |
| 401 | NKEYTGTILF SGEKSLANDP RDFKSTIPQN VNLSAGYLVI |
| | KEGAFVTVSK |
| 451 | FTQSPGSHLV LDLGTKLIAS KEDIAITGLA IDIDSLSSSS |
| | TAAVIKANTA |
| 501 | NKQISVTDSI ELTSPTGNAY EDLRMRNSQT FPLLSLEPGA |
| | GGSVTVTAGD |
| 551 | FLPVSPHYGF QGNWKLAWTG TGNKVGEFFW DKINYKPRPE |
| | KEGNLVPNIL |
| 601 | WGNAVDVRSL MQVQETHASS LQTDRGLWID GIGNFFHVSA |
| | SEDNIRYRHN |
| 651 | SGGYVLSVNN EITPKHYTSM AFSQLFSRDK DYAVSNNEYR |
| | MYLGSYLYQY |
| 701 | TTSLGNIFRY ASRNFNVNVG ILSRRFLQNP LMIFHFLCAY |
| | GHATNDMKTD |
| 751 | YANFPMVKNS WRNNCWAIEC GGSMPLLVFE NGRLFQGAIP |
| | FMKLQLVYAY |
| 801 | QGDFKETTAD GRRFSNGSLT SISVPLGIRF EKLALSQDVL |
| | YDFSFSYIPD |
| 851 | IFRKDPSCEA ALVISGDSWL VPAAHVSRHA FVGSGTGRYH |
| | FNDYTELLCR |
| 901 | GSIECRPHAR NYNINCGSKF RF* |

A predicted signal peptide is highlighted.

The cp6260 nucleotide sequence <SEQ ID 42> is:

| | |
|---|---|
| 1 | ATGCGATTTT CGCTCTGCGG ATTTCCTCTA GTTTTTTCTT |
| | TTACATTGCT |
| 51 | CTCAGTCTTC GACACTTCTT TGAGTGCTAC TACGATTTCT |
| | TTAACCCCAG |
| 101 | AAGATAGTTT TCATGGAGAT AGTCAGAATG CAGAACGTTC |
| | TTATAATGTT |
| 151 | CAAGCTGGGG ATGTCTATAG CCTTACTGGT GATGTCTCAA |
| | TATCTAACGT |
| 201 | CGATAACTCT GCATTAAATA AAGCCTGCTT CAATGTGACC |
| | TCAGGAAGTG |
| 251 | TGACGTTCGC AGGAAATCAT CATGGGTTAT ATTTTAATAA |
| | TATTTCCTCA |
| 301 | GGAACTACAA AGGAAGGGGC TGTACTTTGT TGCCAAGATC |
| | CTCAAGCAAC |
| 351 | GGCACGTTTT TCTGGGTTCT CCACGCTCTC TTTTATTCAG |
| | AGCCCCGGAG |
| 401 | ATATTAAAGA ACAGGGATGT CTCTATTCAA AAAAGTCACT |
| | TATGCTCTTA |
| 451 | AACAATTATG TAGTGCGTTT TGAACAAAAC CAAAGTAAGA |
| | CTAAAGGCGG |
| 501 | AGCTATTAGT GGGGCGAATG TTACTATAGT AGGCAACTAC |
| | GATTCCGTCT |
| 551 | CTTTCTATCA GAATGCAGCC ACTTTTGGAG GTGCTATCCA |
| | TTCTTCAGGT |
| 601 | CCCCTACAGA TTGCAGTAAA TCAGGCAGAG ATAAGATTTG |
| | CACAAAATAC |
| 651 | TGCCAAGAAT GGTTCTGGAG GGGCTTTGTA CTCCGATGGT |
| | GATATTGATA |
| 701 | TTGATCAGAA TGCTTATGTT CTATTTCGAG AAAATGAGGC |
| | ATTGACTACT |
| 751 | GCTATAGGTA AGGGAGGGGC TGTCTGTTGT CTTCCCACTT |
| | CAGGAAGTAG |
| 801 | TACTCCAGTT CCTATTGTGA CTTTCTCTGA CAATAAACAG |
| | TTAGTCTTTG |
| 851 | AAAGAAACCA TTCCATAATG GGTGGCGGAG CCATTTATGC |
| | TAGGAAACTT |
| 901 | AGCATCTCTT CAGGAGGTCC TACTCTATTT ATCAATAATA |
| | TATCATATGC |
| 951 | AAATTCGCAA AATTTAGGTG GAGCTATTGC CATTGATACT |
| | GGAGGGGAGA |
| 1001 | TCAGTTTATC AGCAGAGAAA GGAACAATTA CATTCCAAGG |
| | AAACCGGACG |
| 1051 | AGCTTACCGT TTTTGAATGG CATCCATCTT TTACAAAATG |
| | CTAAATTCCT |
| 1101 | GAAATTACAG GCGAGAAATG GATACTCTAT AGAATTTTAT |
| | GATCCTATTA |
| 1151 | CTTCTGAAGC AGATGGGTCT ACCCAATTGA ATATCAACGG |
| | AGATCCTAAA |
| 1201 | AATAAAGAGT ACACAGGGAC CATACTCTTT TCTGGAGAAA |
| | AGAGTCTAGC |
| 1251 | AAACGATCCT AGGGATTTTA AATCTACAAT CCCTCAGAAC |
| | GTCAACCTGT |
| 1301 | CTGCAGGATA CTTAGTTATT AAAGAGGGGG CCGAAGTCAC |
| | AGTTTCAAAA |
| 1351 | TTCACGCAGT CTCCAGGATC GCATTTAGTT TTAGATTTAG |
| | GAACCAAACT |
| 1401 | GATAGCCTCT AAGGAAGACA TTGCCATCAC AGGCCTCGCG |
| | ATAGATATAG |
| 1451 | ATAGCTTAAG CTCATCCTCA ACAGCAGCTG TTATTAAAGC |
| | AAACACCGCA |
| 1501 | AATAAACAGA TATCCGTGAC GGACTCTATA GAACTTATCT |
| | CGCCTACTGG |
| 1551 | CAATGCCTAT GAAGATCTCA GAATGAGAAA TTCACAGACG |
| | TTCCCTCTGC |
| 1601 | TCTCTTTAGA GCCTGGAGCC GGGGGTAGTG TGACTGTAAC |
| | TGCTGGAGAT |
| 1651 | TTCCTACCGG TAAGTCCCCA TTATGGTTTT CAAGGCAATT |
| | GGAAATTAGC |
| 1701 | TTGGACAGGA ACTGGAAACA AAGTTGGAGA ATTCTTCTGG |
| | GATAAAATAA |
| 1751 | ATTATAAGCC TAGACCTGAA AAAGAAGGAA ATTTAGTTCC |
| | TAATATCTTG |
| 1801 | TGGGGGAATG CTGTAGATGT CAGATCCTTA ATGCAGGTTC |
| | AAGAGACCCA |
| 1851 | TGCATCGAGC TTACAGACAG ATCGAGGGCT GTGGATCGAT |
| | GGAATTGGGA |
| 1901 | ATTTCTTCCA TGTATCTGCC TCCGAAGACA ATATAAGGTA |
| | CCGTCATAAC |
| 1951 | AGCGGTGGAT ATGTTCTATC TGTAAATAAT GAGATCACAC |
| | CTAAGCACTA |
| 2001 | TACTTCGATG GCATTTTCCC AACTCTTTAG TAGAGACAAG |
| | GACTATGCGG |
| 2051 | TTTCCAACAA CGAATACAGA ATGTATTTAG GATCGTATCT |
| | CTATCAATAT |
| 2101 | ACAACCTCCC TAGGGAATAT TTTCCGTTAT GCTTCGCGTA |
| | ACCCTAATGT |
| 2151 | AAACGTCGGG ATTCTCTCAA GAAGGTTTCT TCAAAATCCT |
| | CTTATGATTT |
| 2201 | TTCATTTTTT GTGTGCTTAT GGTCATGCCA CCAATGATAT |
| | GAAAACAGAC |
| 2251 | TACGCAAATT TCCCTATGGT GAAAAACAGC TGGAGAAACA |
| | ATTGTTGGGC |
| 2301 | TATAGAGTGC GGAGGGAGCA TGCCTCTATT GGTATTTGAG |
| | AACGGAAGAC |
| 2351 | TTTTCCAAGG TGCCATCCCA TTTATGAAAC TACAATTAGT |
| | TTATGCTTAT |
| 2401 | CAGGGAGATT TCAAAGAGAC GACTGCAGAT GGCCGTAGAT |
| | TTAGTAATGG |
| 2451 | GAGTTTAACA TCGATTTCTG TACCTCTAGG CATACGCTTT |
| | GAGAAGCTGG |
| 2501 | CACTTTCTCA GGATGTACTC TATGACTTTA GTTTCTCCTA |
| | TATTCCTGAT |
| 2551 | ATTTTCCGTA AGGATCCCTC ATGTGAAGCT GCTCTGGTGA |
| | TTAGCGGAGA |
| 2601 | CTCCTGGCTT GTTCCGGCAG CACACGTATC AAGACATGCT |
| | TTTGTAGGGA |
| 2651 | GTGGAACGGG TCGGTATCAC TTTAACGACT ATACTGAGCT |
| | CTTATGTCGA |
| 2701 | GGAAGTATAG AATGCCGCCC CCATGCTAGG AATTATAATA |
| | TAAACTGTGG |
| 2751 | AAGCAAATTT CGTTTTTAG |

The PSORT algorithm predicts an outer membrane location (0.921).

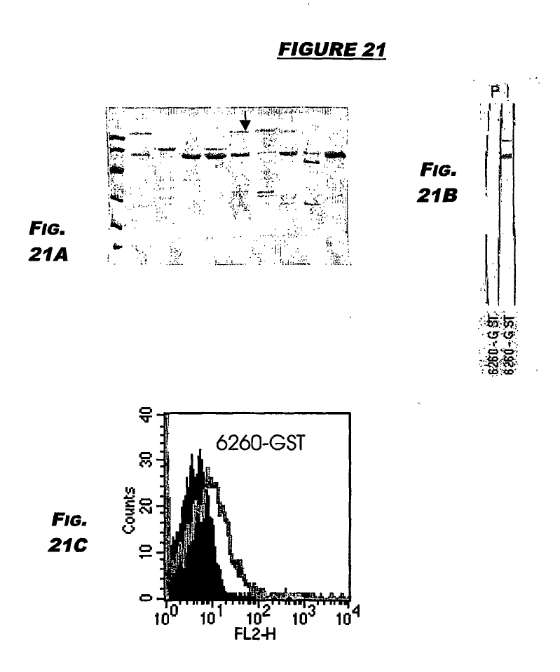

The protein was expressed in E. coli and purified both as a his-tag and GST-fusion product. The GST-fusion is shown in FIG. 21A. This recombinant protein was used to immunise mice, whose sera were used in a Western blot (FIG. 21B) and for FACS analysis (FIG. 21C).

This protein also showed good cross-reactivity with human sera, including sera from patients with pneumonitis.

These experiments show that cp6260 is a surface-exposed and immunoaccessible protein, and that it is a useful immunogen. These properties are not evident from the sequence alone.

### Example 22

The following C. pneumoniae protein (PID 4376456) was expressed <SEQ ID 43; cp6456>:

| | |
|---|---|
| 1 | MSSPVNNTPS APNIPIPAPT TPGIPTTKPR SSFIEKVIIV |
| | AKYILFAIAA |
| 51 | TSGALGTILG LSGALTPGIG IALLVIFFVS MVLLGLILKD |
| | SISGGEERRL |
| 101 | REEVSRFTSE NQRLTVITTT LETEVKDLKA AKDQLTLEIE |
| | AFRNENGNLK |
| 151 | TTAEDLEEQV SKLSEQLEAL ERINQLIQAN AGDAQEISSE |
| | LKKLISGWDS |
| 201 | KVVEQINTSI QALKVLLGQE WVQEAQTHVK AMQEQIQALQ |
| | AEILGMHNQS |
| 251 | TALQKSVENL LVQDQALTRV VGELLESENK LSQACSALRQ |
| | EIEKLAQHET |
| 301 | SLQQRIDAML AQEQNLAEQV TALEKMKQEA QKAESEFIAC |
| | VRDRTFGRRE |
| 351 | TPPPTTPVVE GDESQEEDEG GTPPVSQPSS PVDRATGDGQ * |

The cp6456 nucleotide sequence <SEQ ID 44> is:

| | |
|---|---|
| 1 | ATGTCATCTC CTGTAAATAA CACACCCTCA GCACCAAACA |
| | TTCCAATACC |
| 51 | AGCGCCCACG ACTCCAGGTA TTCCTACAAC AAAACCTCGT |
| | TCTAGTTTCA |
| 101 | TTGAAAAGGT TATCATTGTA GCTAAGTACA TACTATTTGC |
| | AATTGCAGCC |
| 151 | ACATCAGGAG CACTCGGAAC AATTCTAGGT CTATCTGGAG |
| | CGCTAACCCC |
| 201 | AGGAATAGGT ATTGCCCTTC TTGTTATCTT CTTTGTTTCT |
| | ATGGTGCTTT |
| 251 | TAGGTTTAAT CCTTAAAGAT TCTATAAGTG GAGGAGAAGA |
| | ACGCAGGCTC |
| 301 | AGAGAAGAGG TCTCTCGATT TACAGGTGAG AATCAACGGT |
| | TGACAGTCAT |
| 351 | AACCACAACA CTTGAGACTG AAGTAAAGGA TTTAAAAGCA |
| | GCTAAAGATC |
| 401 | AACTTACACT TGAAATCGAA GCATTTAGAA ATGAAAACGG |
| | TAATTTAAAA |
| 451 | ACAACTGCTG AGGACTTAGA AGAGCAGGTT TCTAAACTTA |
| | GCGAACAATT |
| 501 | AGAAGCACTA GAGCGAATTA ATCAACTTAT CCAAGCAAAC |
| | GCTGGAGATG |
| 551 | CTCAAGAAAT TTCGTCTGAA CTAAAGAAAT TAATAAGCGG |
| | TTGGGATTCC |
| 601 | AAAGTTGTTG AACAGATAAA TACTTCTATT CAAGCATTGA |
| | AAGTGTTATT |
| 651 | GGGTCAAGAG TGGGTGCAAG AGGCTCAAAC ACACGTTAAA |
| | GCAATGCAAG |
| 701 | AGCAAATTCA AGCATTGCAA GCTGAAATTC TAGGAATGCA |
| | CAATCAATCT |
| 751 | ACAGCATTGC AAAAGTCAGT TGAGAATCTA TTAGTACAAG |
| | ATCAAGCTCT |
| 801 | AACAAGAGTA GTAGGTGAGT TGTTAGAGTC TGAGAACAAG |
| | CTAAGCCAAG |
| 851 | CTTGTTCTGC GCTACGTCAA GAAATAGAAA AGTTGGCCCA |
| | ACATGAAACA |
| 901 | TCTTTGCAAC AACGTATTGA TGCGATGCTA GCCCAAGAGC |
| | AAAATTTGGC |
| 951 | AGAGCAGGTC ACAGCCCTTG AAAAAATGAA ACAAGAAGCT |
| | CAGAAGGCTG |
| 1001 | AGTCCGAGTT CATTGCTTGT GTACGTGATC GAACTTTCGG |
| | ACGTCGTGAA |
| 1051 | ACACCTCCAC CAACAACACC TGTAGTTGAA GGTGATGAAA |
| | GTCAAGAAGA |
| 1101 | AGACGAAGGA GGTACTCCCC CAGTATCACA ACCATCTTCA |
| | CCCGTAGATA |
| 1151 | GAGCAACAGG AGATGGTCAG TAA |

The PSORT algorithm predicts inner membrane (0.127).

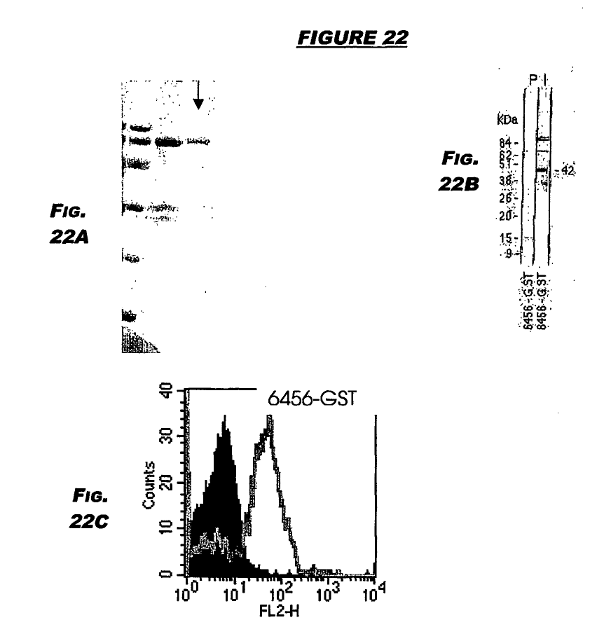

The protein was expressed in E. coli and purified as a GST-fusion product, as shown in FIG. 22A. The recombinant protein was used to immunise mice, whose sera were used in a Western blot (FIG. 22B) and for FACS analysis (FIG. 22C). A his-tag protein was also expressed.

These experiments show that cp6456 is a surface-exposed and immunoaccessible protein, and that it is a useful immunogen. These properties are not evident from the sequence alone.

### Example 23

The following C. pneumoniae protein (PID 4376729) was expressed <SEQ ID 45; cp6729>:

| | |
|---|---|
| 1 | MKIPLHKLLI SSTLVTRILL SIATYGADAS LSPTDSFDGA |
| | GGSTFTPKST |
| 51 | ADANGTNYVL SGNVYINDAG KGTALTGCCF TETTGDLTFT |
| | GKGYSFSFNT |
| 101 | VDAGSNAGAA ASTTADKALT FTGFSNLSFI AAPGTTVASG |
| | KSTLSSAGAL |
| 151 | NLTDNGTILF SQNVSNEANN NGGAITTKTL SISGNTSSIT |
| | FTSNSAKKLG |
| 201 | GAIYSSAAAS ISGNTGQLVF MNNKGETGGG ALGFEASSSI |
| | TQNSSLFFSG |
| 251 | NTATDAAGKG GAIYCEKTGE TPTLTISGNK SLTFAENSSV |
| | TQGGAICAHG |
| 301 | LDLSAAGPTL FSNNRCGNTA AGKGGAIAIA DSGSLSLSAN |
| | QGDITFLGNT |
| 351 | LTSTSAPTST RNAIYLGSSA KITNLRAAQG QSIYFYDPIA |
| | SNTTGASDVL |
| 401 | TINQPDSNSP IDYSGTIVFS GEKLSADEAK AADNFTSILK |
| | QPLALASGTL |
| 451 | ALKGNVELDV NGFTQTEGST LLMQPGTKLK ADTEAISLTK |
| | LVVDLSALEG |
| 501 | NKSVSIETAG ANKTITLTSP LVFQDSSGNF YESHTINQAF |
| | TQPLVVFTAA |
| 551 | TAASDIYIDA LLTSPVQTPE PHYGYQGHWE ATWADTSTAK |
| | SGTMTWVTTG |
| 601 | YNPNPERRAS VVPDSLWASF TDIRTLQQIM TSQANSIYQQ |
| | RGLWASGTAN |
| 651 | FFHKDKSGTN QAFRHKSYGY IVGGSAEDFS ENIFSVAFCQ |
| | LFGKDKDLFI |
| 701 | VENTSHNYLA SLYLQHRAFL GGLPMPSFGS ITDMLKDIPL |
| | ILNAQLSYSY |
| 751 | TKNDMDTRYT SYPEAQGSWT NNSGALELGG SLALYLPKEA |
| | PFFQGYFPFL |
| 801 | KFQAVYSRQQ NFKESGAEAR AFDDGDLVNC SIPVGIRLEK |
| | ISEDEKNNFE |
| 851 | ISLAYIGDVY RKNPRSRTSL MVSGASWTSL CKNLARQAFL |
| | ASAGSHLTLS |
| 901 | PHVELSGEAA YELRGSAHIY NVDCGLRYSF * |

A predicted signal peptide is highlighted.

The cp6729 nucleotide sequence <SEQ ID 46> is:

| | |
|---|---|
| 1 | ATGAAAATAC CCTTGCACAA ACTCCTGATC TCTTCGACTC |
| | TTGTCACTCC |
| 51 | CATTCTATTG AGCATTGCAA CTTACGGAGC AGATGCTTCT |
| | TTATCCCCTA |
| 101 | CAGATAGCTT TGATGGAGCG GGCGGCTCTA CATTTACTCC |
| | AAAATCTACA |
| 151 | GCAGATGCCA ATGGAACGAA CTATGTCTTA TCAGGAAATG |
| | TCTATATAAA |
| 201 | CGATGCTGGG AAAGGCACAG CATTAACAGG CTGCTGCTTT |
| | ACAGAAACTA |
| 251 | CGGGTGATCT GACATTTACT GGAAAGGGAT ACTCATTTTC |
| | ATTCAACACG |
| 301 | GTAGATGCGG GTTCGAATGC AGGAGCTGCG GCAAGCACAA |
| | CTGCTGATAA |
| 351 | AGCCCTAACA TTCACAGGAT TTTCTAACCT TTCCTTCATT |
| | GCAGCTCCTG |
| 401 | GAACTACAGT TGCTTCAGGA AAAAGTACTT TAAGTTCTGC |
| | AGGAGCCTTA |
| 451 | AATCTTACCG ATAATGGAAC GATTCTCTTT AGCCAAAACG |
| | TCTCCAATGA |
| 501 | AGCTAATAAC AATGGCGGAG CGATCACCAC AAAAACTCTT |
| | TCTATTTCTG |
| 551 | GGAATACCTC TTCTATAACC TTCACTAGTA ATAGCGCAAA |
| | AAAATTAGGT |
| 601 | GGAGCGATCT ATAGCTCTGC GGCTGCAAGT ATTTCAGGAA |
| | ACACCGGCCA |
| 651 | GTTAGTCTTT ATGAATAATA AAGGAGAAAC TGGGGGTGGG |
| | GCTCTGGGCT |
| 701 | TTGAAGCCAG CTCCTCGATT ACTCAAAATA GCTCCCTTTT |
| | CTTCTCTGGA |
| 751 | AACACTGCAA CAGATGCTGC AGGCAAGGGC GGGGCCATTT |
| | ATTGTGAAAA |
| 801 | AACAGGAGAG ACTCCTACTC TTACTATCTC TGGAAATAAA |
| | AGTCTGACCT |
| 851 | TCGCCGAGAA CTCTTCAGTA ACTCAAGGCG GAGCAATCTG |
| | TGCCCATGGT |
| 901 | CTAGATCTTT CCGCTGCTGG CCCTACCCTA TTTTCAAATA |
| | ATAGATGCGG |
| 951 | GAACACAGCT GCAGGCAAGG GCGGCGCTAT TGCAATTGCC |
| | GACTCTGGAT |
| 1001 | CTTTAAGTCT CTCTGCAAAT CAAGGAGACA TCACGTTCCT |
| | TGGCAACACT |
| 1051 | CTAACCTCAA CCTCCGCGCC AACATCGACA CGGAATGCTA |
| | TCTACCTGGG |
| 1101 | ATCGTCAGCA AAAATTACGA ACTTAAGGGC AGCCCAAGGC |
| | CAATCTATCT |
| 1151 | ATTTCTATGA TCCGATTGCA TCTAACACCA CAGGAGCTTC |
| | AGACGTTCTG |
| 1201 | ACCATCAACC AACCGGATAG CAACTCGCCT TTAGATTATT |
| | CAGGAACGAT |
| 1251 | TGTATTTTCT GGGGAAAAGC TCTCTGCAGA TGAAGCGAAA |
| | GCTGCTGATA |
| 1301 | ACTTCACATC TATATTAAAG CAACCATTGG CTCTAGCCTC |
| | TGGAACCTTA |
| 1351 | GCACTCAAAG GAAATGTCGA GTTAGATGTC AATGGTTTCA |
| | CACAGACTGA |
| 1401 | AGGCTCTACA CTCCTCATGC AACCAGGAAC AAAGCTCAAA |
| | GCAGATACTG |
| 1451 | AAGCTATCAG TCTTACCAAA CTTGTCGTTG ATCTTTCTGC |
| | CTTAGAGGGA |
| 1501 | AATAAGAGTG TGTCCATTGA AACAGCAGGA GCCAACAAAA |
| | CTATAACTCT |
| 1551 | AACCTCTCCT CTTGTTTTCC AAGATAGTAG CGGCAATTTT |
| | TATGAAAGCC |
| 1601 | ATACGATAAA CCAAGCCTTC ACGCAGCCTT TGGTGGTATT |
| | CACTGCTGCT |
| 1651 | ACTGCTGCTA GCGATATTTA TATCGATGCG CTTCTCACTT |
| | CTCCAGTACA |
| 1701 | AACTCCAGAA CCTCATTACG GGTATCAGGG ACATTGGGAA |
| | GCCACTTGGG |
| 1751 | CAGACACATC AACTGCAAAA TCAGGAACTA TGACTTGGGT |
| | AACTACGGGC |
| 1801 | TACAACCCTA ATCCTGAGCG TAGAGCTTCC GTAGTTCCCG |
| | ATTCATTATG |
| 1851 | GGCATCCTTT ACTGACATTC GCACTCTACA GCAGATCATG |
| | ACATCTCAAG |
| 1901 | CGAATAGTAT CTATCAGCAA CGAGGACTCT GGGCATCAGG |
| | AACTGCGAAT |
| 1951 | TTCTTCCATA AGGATAAATC AGGAACTAAC CAAGCATTCC |
| | GACATAAAAG |
| 2001 | CTACGGCTAT ATTGTTGGAG GAAGTGCTGA AGATTTTTCT |
| | GAAAATATCT |
| 2051 | TCAGTGTAGC TTTCTGCCAG CTCTTCGGTA AAGATAAAGA |
| | CCTGTTTATA |
| 2101 | GTTGAAAATA CCTCTCATAA CTATTTAGCG TCGCTATACC |
| | TGCAACATCG |
| 2151 | AGCATTCCTA GGAGGACTTC CCATGCCCTC ATTTGGAAGT |
| | ATCACCGACA |
| 2201 | TGCTGAAAGA TATTCCTCTC ATTTTGAATG CCCAGCTAAG |
| | CTACAGCTAC |
| 2251 | ACTAAAAATG ATATGGATAC TCGCTATACT TCCTATCCTG |
| | AAGCTCAAGG |
| 2301 | CTCTTGGACC AATAACTCTG GGGCTCTAGA GCTCGGAGGA |
| | TCTCTGGCTC |
| 2351 | TATATCTCCC TAAAGAAGCA CCGTTCTTCC AGGGATATTT |
| | CCCCTTCTTA |
| 2401 | AAGTTCCAGG CAGTCTACAG CCGCCAACAA AACTTTAAAG |
| | AGAGTGGCGC |
| 2451 | TGAAGCCCGT GCTTTTGATG ATGGAGACCT AGTGAACTGC |
| | TCTATCCCTG |
| 2501 | TCGGCATTCG GTTAGAAAAA ATCTCCGAAG ATGAAAAAAA |
| | TAATTTCGAG |
| 2551 | ATTTCTCTAG CCTACATTGG TGATGTGTAT CGTAAAAATC |
| | CCCGTTCGCG |
| 2601 | TACTTCTCTA ATGGTCAGTG GAGCCTCTTG GACTTCGCTA |
| | TGTAAAAACC |
| 2651 | TCGCACGACA AGCCTTCTTA GCAAGTGCTG GAAGCCATCT |
| | GACTCTCTCC |
| 2701 | CCTCATGTAG AACTCTCTGG GGAAGCTGCT TATGAGCTTC |
| | GTGGCTCAGC |
| 2751 | ACACATCTAC AATGTAGATT GTGGGCTAAG ATACTCATTC |
| | TAG |

The PSORT algorithm predicts outer membrane (0.927).

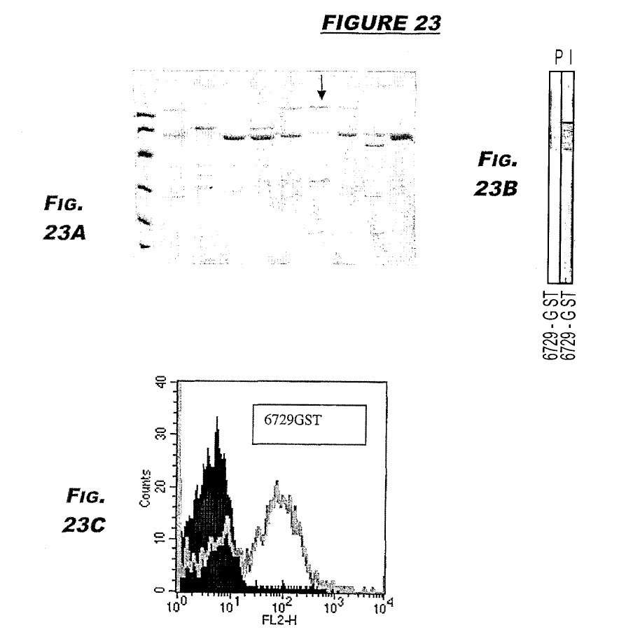

The protein was expressed in E. coli and purified as a GST-fusion product, as shown in FIG. 23A. The recombinant protein was used to immunise mice, whose sera were used in a Western blot (FIG. 23B) and for FACS analysis (FIG. 23C). A his-tag protein was also expressed.

The cp6729 protein was also identified in the 2D-PAGE experiment (Cpn0446) and showed good cross-reactivity with human sera, including sera from patients with pneumonitis.

These experiments show that cp6729 is a surface-exposed and immunoaccessible protein, and that it is a useful immunogen. These properties are not evident from the sequence alone.

### Example 24

The following C. pneumoniae protein (PID 4376849) was expressed <SEQ ID 47; cp6849>:

| | |
|---|---|
| 1 | MSKLIRRVVT VLALTSMASC FASGGIEAAV AESLITKIVA |
| | SAETKPAPVP |
| 51 | MTAKKVRLVR RNKQPVEQKS RGAFCDKEFY PCEEGRCQPV |
| | EAQQESCYGR |
| 101 | LYSVKVNDDC NVEICQSVPE YATVGSPYPI EILAIGKKDC |
| | VDVVITQQLP |
| 151 | CEAEFVSSDP ETTPTSDGKL VWKIDRLGAG DKCKITVWVK |
| | PLKEGCCFTA |
| 201 | ATVCACPELR SYTKCGQPAI CIKQEGPDCA CLRCPVCYKI |
| | EVVNTGSAIA |
| 251 | RNVTVDNPVP DGYSHASGQR VLSFNLGDMR PGDKKVFTVE |
| | FCPQRRGQIT |
| 301 | NVATVTYCGG HKCSANVTTV VNEPCVQVNI SGADWSYVCK |
| | PVEYSISVSN |
| 351 | PGDLVLHDVV IQDTLPSGVT VLEAPGGEIC CNKVVWRIKE |
| | MCPGETLQFK |
| 401 | LVVKAQVPGR FTNQVAVTSE SNCGTCTSCA ETTTHWKGLA |
| | ATHMCVLDTN |
| 451 | DPICVGENTV YRICVTNRGS AEDTNVSLIL KFSKELQPIA |
| | SSGPTKGTIS |
| 501 | GNTVVFDALP KLGSKESVEF SVTLKGIAPG DARGEAILSS |
| | DTLTSPVSDT |
| 551 | ENTHVY* |

A predicted signal peptide is highlighted.

The cp6849 nucleotide sequence <SEQ ID 48> is:

| | |
|---|---|
| 1 | ATGTCCAAAC TCATCAGACG AGTAGTTACG GTCCTTGCGC |
| | TAACGAGTAT |
| 51 | GGCGAGTTGC TTTGCCAGCG GGGGTATAGA GGCCGCTGTA |
| | GCAGAGTCTC |
| 101 | TGATTACTAA GATCGTCGCT AGTGCGGAAA CAAAGCCAGC |
| | ACCTGTTCCT |
| 151 | ATGACAGCGA AGAAGGTTAG ACTTGTCCGT AGAAATAAAC |
| | AACCAGTTGA |
| 201 | ACAAAAAAGC CGTGGTGCTT TTTGTGATAA AGAATTTTAT |
| | CCCTGTGAAG |
| 251 | AGGGACGATG TCAACCTGTA GAGGCTCAGC AAGAGTCTTG |
| | CTACGGAAGA |
| 301 | TTGTATTCTG TAAAAGTAAA CGATGATTGC AACGTAGAAA |
| | TTTGCCAGTC |
| 351 | CGTTCCAGAA TACGCTACTG TAGGATCTCC TTACCCTATT |
| | GAAATCCTTG |
| 401 | CTATAGGCAA AAAAGATTGT GTTGATGTTG TGATTACACA |
| | ACAGCTACCT |
| 451 | TGCGAAGCTG AATTCGTAAG CAGTGATCCA GAAACAACTC |
| | CTACAAGTGA |
| 501 | TGGGAAATTA GTCTGGAAAA TCGATCGCCT GGGTGCAGGA |
| | GATAAATGCA |
| 551 | AAATTACTGT ATGGGTAAAA CCTCTTAAAG AAGGTTGCTG |
| | CTTCACAGCT |
| 601 | GCTACTGTAT GTGCTTGCCC AGAGCTCCGT TCTTATACTA |
| | AATGCGGTCA |
| 651 | ACCAGCCATT TGTATTAAGC AAGAAGGACC TGACTGTGCT |
| | TGCCTAAGAT |
| 701 | GCCCTGTATG CTACAAAATC GAAGTAGTGA ACACAGGATC |
| | TGCTATTGCC |
| 751 | CGTAACGTAA CTGTAGATAA TCCTGTTCCC GATGGCTATT |
| | CTCATGCATC |
| 801 | TGGTCAAAGA GTTCTCTCTT TTAACTTAGG AGACATGAGA |
| | CCTGGCGATA |
| 851 | AAAAGGTATT TACAGTTGAG TTCTGCCCTC AAAGAAGAGG |
| | TCAAATCACT |
| 901 | AACGTTGCTA CTGTAACTTA CTGCGGTGGA CACAAATGTT |
| | CTGCAAATGT |
| 951 | AACTACAGTT GTTAATGAGC CTTGTGTACA AGTAAATATC |
| | TCTGGTGCTG |
| 1001 | ATTGGTCTTA CGTATGTAAA CCTGTGGAGT ACTCTATCTC |
| | AGTATCGAAT |
| 1051 | CCTGGAGACT TGGTTCTTCA TGATGTCGTG ATCCAAGATA |
| | CACTCCCTTC |
| 1101 | TGGTGTTACA GTACTCGAAG CTCCTGGTGG AGAGATCTGC |
| | TGTAATAAAG |
| 1151 | TTGTTTGGCG TATTAAAGAA ATGTGCCCAG GAGAAACCCT |
| | CCAGTTTAAA |
| 1201 | CTTGTAGTGA AAGCTCAAGT TCCTGGAAGA TTCACAAATC |
| | AAGTTGCAGT |
| 1251 | AACTAGTGAG TCTAACTGCG GAACATGTAC ATCTTGCGCA |
| | GAAACAACAA |
| 1301 | CACATTGGAA AGGTCTTGCA GCTACCCATA TGTGCGTATT |
| | AGACACAAAT |
| 1351 | GATCCTATCT GTGTAGGAGA AAATACTGTC TATCGTATCT |
| | GTGTAACTAA |
| 1401 | CCGTGGTTCT GCTGAAGATA CTAACGTATC TTTAATCTTG |
| | AAGTTCTCAA |
| 1451 | AAGAACTTCA GCCAATAGCT TCTTCAGGTC CAACTAAAGG |
| | AACGATTTCA |
| 1501 | GGTAATACCG TTGTTTTCGA CGCTTTACCT AAACTCGGTT |
| | CTAAGGAATC |
| 1551 | TGTAGAGTTT TCTGTTACCT TGAAAGGTAT TGCTCCCGGA |
| | GATGCTCGCG |
| 1601 | GCGAAGCTAT TCTTTCTTCT GATACACTGA CTTCACCAGT |
| | ATCAGACACA |
| 1651 | GAAAATACCC ACGTGTATTA A |

The PSORT algorithm predicts periplasmic space (0.93).

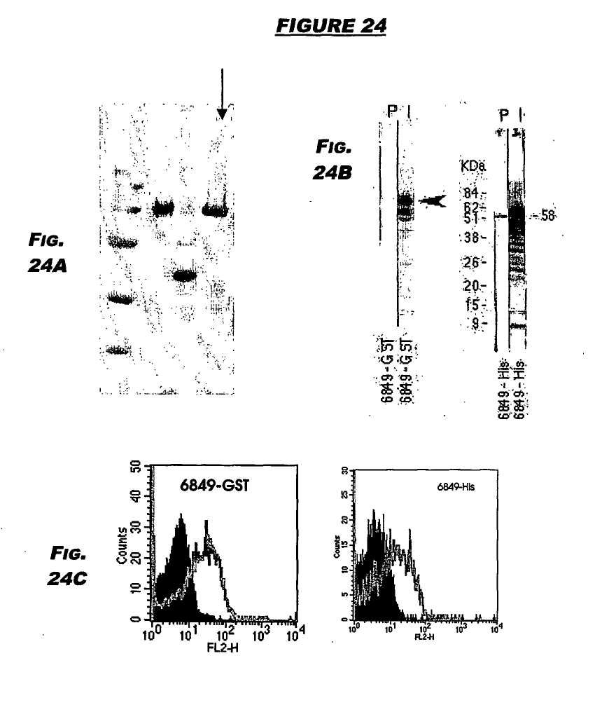

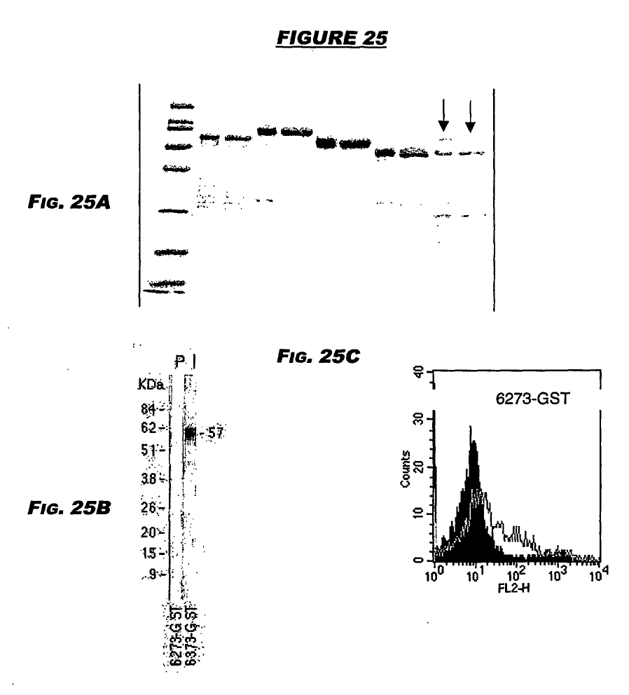

The protein was expressed in E. coli and purified as a GST-fusion product, as shown in FIG. 24A, and also as a his-tag protein. The recombinant proteins were used to immunise mice, whose sera were used in a Western blot (FIG. 24B) and for FACS analysis (FIG. 24C).

The cp6849 protein was also identified in the 2D-PAGE experiment (Cpn0557).

These experiments show that cp6849 is a surface-exposed and immunoaccessible protein, and that it is a useful immunogen. These properties are not evident from the sequence alone.

### Example 25

The following C. pneumoniae protein (PID 4376273) was expressed <SEQ ID 49; cp6273>:

| | |
|---|---|
| 1 | MGLFHLTLFG LLLCSLPISL VAKFPESVGH KILYISTQST |
| | QQALATYLEA |
| 51 | LDAYGDHDFF VLRKIGEDYL KQSIHSSDPQ TRKSTIIGAG |
| | LAGSSEALDV |
| 101 | LSQAMETADP LQQLLVLSAV SGHLGKTSDD LLFKALASPY |
| | PVIRLEAAYR |
| 151 | LANLKNTKVI DHLHSFIHKL PEEIQCLSAA IFLRLETEES |
| | DAYIRDLLAA |
| 201 | KKSAIRSATA LQIGEYQQKR FLPTLRNLLT SASPQDQEAI |
| | LYALGKLKDG |
| 251 | QSYYNIKKQL QKPDVDVTLA AAQALIALGK EEDALPVIKK |
| | QALEERPRAL |
| 301 | YALRHLPSEI GIPIALPIFL KTKNSEAKLN VALALLELGC |
| | DTPKLLEYIT |
| 351 | ERLVQPHYNE TLALSFSKGR TLQNWKRVNI IVPQDPQERE |
| | RLLSTTRGLE |
| 401 | EQILTFLFRL PKEAYLPCIY KLLASQKTQL ATTAISFLSH |
| | TSHQEALDLL |
| 451 | FQAAKLPGEP IIRAYADLAI YNLTKDPEKK RSLHDYAKKL |
| | IQETLLFVDT |
| 501 | ENQRPHPSMP YLRYQVTPES RYKLMLDILE TLATSKSSED |
| | IRLLIQLMTE |
| 551 | GDAKNPPVLA GLLIKIVE* |

A predicted signal peptide is highlighted.

The cp6273 nucleotide sequence <SEQ ID 50> is:

| | |
|---|---|
| 1 | ATGGGACTAT TCCATCCAAC TCTCTTTGGA CTTTTATTGT |
| | GTAGTCTTCC |
| 51 | CATTTCTCTT GTTGCTAAAT TCCCTGAGTC TGTAGGTCAT |
| | AAGATCCTTT |
| 101 | ATATAAGTAC GCAATCTACA CAGCAGGCCT TAGCAACATA |
| | TCTGGAAGCT |
| 151 | CTAGATGCCT ACGGTGATCA TGACTTCTTC GTTTTAAGAA |
| | AAATCGGAGA |
| 201 | AGACTATCTC AAGCAAAGCA TCCACTCCTC AGATCCGCAA |
| | ACTAGAAAAA |
| 251 | GCACCATCAT TGGAGCAGGC CTGGCGGGAT CTTCAGAAGC |
| | CTTGGACGTG |
| 301 | CTCTCCCAAG CTATGGAAAC TGCAGACCCC CTGCAGCAGC |
| | TACTGGTTTT |
| 351 | ATCGGCAGTC TCAGGACATC TTGGGAAAAC TTCTGACGAC |
| | TTACTGTTTA |
| 401 | AAGCTTTAGC ATCTCCCTAT CCTGTCATCC GCTTAGAAGC |
| | CGCCTATAGA |
| 451 | CTTGCTAATT TGAAGAACAC TAAAGTCATT GATCATCTAC |
| | ATTCTTTCAT |
| 501 | TCATAAGCTT CCCGAAGAAA TCCAATGCCT ATCTGCGGCA |
| | ATATTCCTAC |
| 551 | GCTTGGAGAC TGAAGAATCT GATGCTTATA TTCGGGATCT |
| | CTTAGCTGCC |
| 601 | AAGAAAAGCG CGATTCGGAG TGCCACAGCT TTGCAGATCG |
| | GAGAATACCA |
| 651 | ACAAAAACGC TTTCTTCCGA CACTTAGGAA TTTGCTAACG |
| | AGTGCGTCTC |
| 701 | CTCAAGATCA AGAAGCTATT CTTTATGCTT TAGGGAAGCT |
| | TAAGGATGGT |
| 751 | CAGAGCTACT ACAATATAAA AAAGCAATTG CAGAAGCCTG |
| | ATGTGGATGT |
| 801 | CACTTTAGCA GCAGCTCAAG CTTTAATTGC TTTGGGGAAA |
| | GAAGAGGACG |
| 851 | CTCTTCCCGT GATAAAAAAG CAAGCACTTG AGGAGCGGCC |
| | TCGAGCCCTG |
| 901 | TATGCCTTAC GGCATCTACC CTCTGAGATA GGGATTCCGA |
| | TTGCCCTGCC |
| 951 | GATAGGCCTA AAAACTAAGA ACAGCGAAGC CAAGTTGAAT |
| | GTAGCTTTAG |
| 1001 | CTCTCTTAGA GTTAGGGTGT GACACCCCTA AACTACTGGA |
| | ATACATTACC |
| 1051 | GAAAGGCTTG TCCAACCACA TTATAATGAG ACTCTAGCCT |
| | TGAGTTTCTC |
| 1101 | TAAGGGGCGT ACTTTACAAA ATTGGAAGCG GGTGAACATC |
| | ATAGTCCCTG |
| 1151 | AAGATCCCCA GGAGAGGGAA AGGTTGCTCT CCACAACCCG |
| | AGGTCTTGAA |
| 1201 | GAGCAGATCC TTACGTTTCT CTTCCGCCTA CCTAAAGAAG |
| | CTTACCTCCC |
| 1251 | CTGTATTTAT AAGCTTTTGG CGAGTCAGAA AACTCAGCTT |
| | GCCACTACTG |
| 1301 | CGATTTCTTT TTTAAGTCAC ACCTCACATC AGGAAGCCTT |
| | AGATCTACTT |
| 1351 | TTCCAAGCTG CGAAGCTTCC TGGAGAACCT ATCATCCGCG |
| | CCTATGCAGA |
| 1401 | TCTTGCTATT TATAATCTCA CCAAAGATCC TGAAAAAAAA |
| | CGTTCTCTCC |
| 1451 | ATGATTATGC AAAAAAGCTA ATTCAGGAAA CCTTGTTATT |
| | TGTGGACACG |
| 1501 | GAAAACCAAA GACCCCATCC CAGCATGCCC TATCTACGTT |
| | ATCAGGTCAC |
| 1551 | CCCAGAAAGC CGTACGAAGC TCATGTTGGA TATTCTAGAG |
| | ACACTAGCCA |
| 1601 | CCTCGAAGTC TTCCGAAGAT ATCCGTTTAT TGATACAACT |
| | GATGACGGAA |
| 1651 | GGAGATGCAA AAAATTTCCC AGTCCTTGCA GGCTTACTCA |
| | TAAAAATTGT |
| 1701 | GGAGTAA |

The PSORT algorithm predicts a periplasmic location (0.922).

The protein was expressed in E. coli and purified as a his-tag product and as a GST-fusion product, as shown in FIG. 25A. The recombinant GST-fusion was used to immunise mice, whose sera were used in a Western blot (FIG. 25B) and for FACS analysis (FIG. 25C).

This protein also showed good cross-reactivity with human sera, including sera from patients with pneumonitis.

These experiments show that cp6273 is a surface-exposed and immunoaccessible protein, and that it is a useful immunogen. These properties are not evident from the sequence alone.

### Example 26

The following C. pneumoniae protein (PID 4376735) was expressed <SEQ ID 51; cp6735>:

| | |
|---|---|
| 1 | MTILRNFLTC SALFLALPAA AQVVYLHESD GYNGAINNKS |
| | LEPKITCYPE |
| 51 | GTSYIFLDDV RISNVKHDQE DAGVFINRSG NLFFMGNRCN |
| | FTFHNLMTEG |
| 101 | FGAAISNRVG DTTLTLSNFS YLAFTSAPLL PQGQGAIYSL |
| | GSVMIENSEE |
| 151 | VTFCGNYSSW SGAAIYTPYL LGSKASRPSV NLSGNRYLVF |
| | RDNVSQGYGG |
| 201 | AISTHNLTLT TRGPSCFENN HAYHDVNSNG GAIAIAPGGS |
| | ISISVKSGDL |
| 251 | IFKGNTASQD GNTIHNSIHL QSGAQFKNLR AVSESGVYFY |
| | DPISHSESHK |
| 301 | ITDLVINAPE GKETYEGTIS FSGLCLDDHE VCAENLTSTI |
| | LQDVTLAGGT |
| 351 | LSLSDGVTLQ LHSFKQEASS TLTMSPGTTL LCSGDARVQN |
| | LHILIEDTDN |
| 401 | FVPVRIRAED KDALVSLEKL KVAFEAYWSV YDFPQFKEAF |
| | TIPLLELLGP |
| 451 | SFDSLLLGET TLERTQVTTE NDAVRGFWSL SWEEYPPSLD |
| | KDRRITPTKK |
| 501 | TVFLTWNPEI TSTP* |

A predicted signal peptide is highlighted.

The cp6735 nucleotide sequence <SEQ ID 52> is:

| | |
|---|---|
| 1 | ATGACCATAC TTCGAAATTT TCTTACCTGC TCGGCTTTAT |
| | TCCTCGCTCT |
| 51 | CCCTGCAGCA GCACAAGTTG TATATCTTCA TGAAAGTGAT |
| | GGTTATAACG |
| 101 | GTGCTATCAA TAATAAAAGC TTAGAACCTA AAATTACCTG |
| | TTATCCAGAA |
| 151 | GGAACTTCTT ACATCTTTCT AGATGACGTG AGGATTTCCA |
| | ACGTTAAGCA |
| 201 | TGATCAAGAA GATGCTGGGG TTTTTATAAA TCGATCTGGG |
| | AATCTTTTTT |
| 251 | TCATGGGCAA CCGTTGCAAC TTCACTTTTC ACAACCTTAT |
| | GACCGAGGGT |
| 301 | TTTGGCGCTG CCATTTCGAA CCGCGTTGGA GACACCACTC |
| | TCACTCTCTC |
| 351 | TAATTTTTCT TACTTAGCGT TCACCTCAGC ACCTCTACTA |
| | CCTCAAGGAC |
| 401 | AAGGAGCGAT TTATAGTCTT GGTTCCGTGA TGATCGAAAA |
| | TAGTGAGGAA |
| 451 | GTGACTTTCT GTGGGAACTA CTCTTCGTGG AGTGGAGCTG |
| | CGATTTATAC |
| 501 | TCCCTACCTT TTAGGTTCTA AGGCGAGTCG TCCTTCAGTA |
| | AATCTCAGCG |
| 551 | GGAACCGCTA CCTGGTGTTT AGAGACAATG TGAGCCAAGG |
| | TTATGGCGGC |
| 601 | GCCATATCTA CCCACAATCT CACACTCACG ACTCGAGGAC |
| | CTTCGTGTTT |
| 651 | TGAAAATAAT CATGCTTATC ATGACGTGAA TAGTAATGGA |
| | GGAGCCATTG |
| 701 | CCATTGCTCC TGGAGGATCG ATCTCTATAT CCGTGAAAAG |
| | CGGAGATCTC |
| 751 | ATCTTCAAAG GAAATACAGC ATCACAAGAC GGAAATACAA |
| | TACACAACTC |
| 801 | CATCCATCTG CAATCTGGAG CACAGTTTAA GAACCTACGT |
| | GCTGTTTCAG |
| 851 | AATCCGGAGT TTATTTCTAT GATCCTATAA GCCATAGCGA |
| | GTCGCATAAA |
| 901 | ATTACAGATC TTGTAATCAA TGCTCCTGAA GGAAAGGAAA |
| | CTTATGAAGG |
| 951 | AACAATTAGC TTCTCAGGAC TATGCCTGGA TGATCATGAA |
| | GTTTGTGCGG |
| 1001 | AAAATCTTAC TTCCACAATC CTACAAGATG TCACATTAGC |
| | AGGAGGAACT |
| 1051 | CTCTCTCTAT CGGATGGGGT TACCTTGCAA CGTCATTCTT |
| | TTAAGCAGGA |
| 1101 | ACGAAGCTCT ACGCTTACTA TGTCTCCAGG AACCACTCTG |
| | CTCTGCTCAG |
| 1151 | GAGATGCTCT GGTTCAGAAT CTGCACATCC TGATTGAAGA |
| | TACCGACAAC |
| 1201 | TTTGTTCCTG TAAGGATTCG CGCCGAGGAC AAGGATGCTC |
| | TTGTCTCATT |
| 1251 | AGAAAAACTT AAAGTTGCCT TTGAGGCTTA TTGGTCCGTC |
| | TATGACTTTC |
| 1301 | CTCAATTTAA GGAAGCCTTT ACGATTCCTC TTCTTGAACT |
| | TCTAGGGCCT |
| 1351 | TCTTTTGACA GTCTTCTCCT AGGGGAGACC ACTTTGGAGA |
| | GAACCCAAGT |
| 1401 | CACAACAGAG AATGACGCCG TTCGAGGTTT CTGGTCCCTA |
| | AGCTGGGAAG |
| 1451 | AGTACCCCCC TTCTCTGGAT AAAGACAGAA GGATCACACC |
| | AACTAAGAAA |
| 1501 | ACTGTTTTCC TCACTTGGAA TCCTGAGATC ACTTCTACGC |
| | CATAA |

The PSORT algorithm predicts an outer membrane location (0.922).

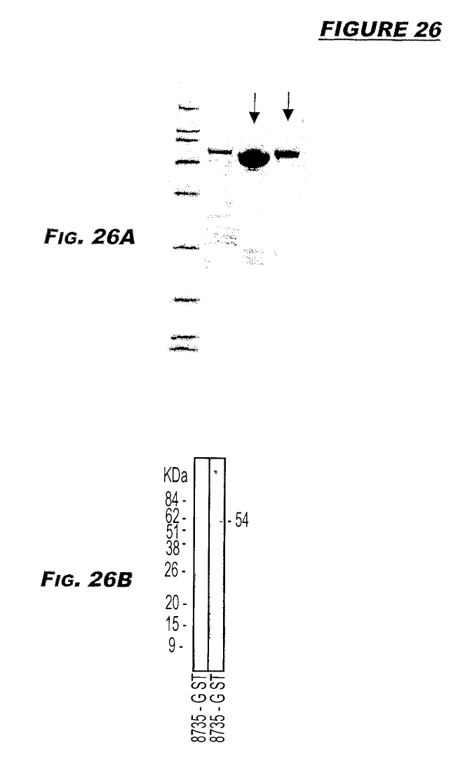

The protein was expressed in E. coli and purified as a as a his-tag product and as a GST-fusion product, as shown in FIG. 26A. The recombinant GST-fusion protein was used to immunise mice, whose sera were used in a Western blot (FIG. 26B).

These experiments show that cp6735 is a surface-exposed and immunoaccessible protein, and that it is a useful immunogen. These properties are not evident from the sequence alone.

### Example 27

The following C. pneumoniae protein (PID 4376784) was expressed <SEQ ID 53; cp6784>:

| | |
|---|---|
| 1 | MNRRKARWVV ALFAMTALIS VGCCPWSQAK SRCSIDKYIP |
| | VVNRLLEVCG |
| 51 | LPEAENVEDL IESSSAWVLT PEERFSGDLV SICQVKDEHA |
| | FYNDLSLLHM |
| 101 | TQAVPSYSAT YDCAVVFGGP LPALRQRLDF LVREWQRGVR |
| | FKKIVFLCGE |
| 151 | RGRYQSIEEQ EHFFDSRYNP SFTEENWESG NRVTPSSEEE |
| | IAKFVWMQML |
| 201 | LPRAWRDSTS GVRVTFLLAK PEENRVVANR KDTLLLFRSY |
| | QEAFPGRVLF |
| 251 | VSSQPFIGLD ACRVGQFFKG ESYDLAGPGF AQGVLKYHWA |
| | PRICLHTLAE |
| 301 | WLKETNGCLN ISEGCFG* |

A predicted signal peptide is highlighted.

The cp6784 nucleotide sequence <SEQ ID 54> is:

| | |
|---|---|
| 1 | ATGAATAGAA GAAAAGCAAG ATGGGTAGTG GCATTGTTCG |
| | CAATGACGGC |
| 51 | GCTCATTTCT GTTGGGTGTT GTCCTTGGTC ACAAGCGAAA |
| | TCAAGATGTT |
| 101 | CTATTGATAA GTATATTCCT GTAGTCAATC GTTTACTAGA |
| | AGTTTGTGGA |
| 151 | CTTCCTGAAG CTGAGAATGT TGAGGATTTA ATCGAGTCCT |
| | CGTCTGCTTG |
| 201 | GGTACTGACT CCTGAAGAAC GTTTTTCTGG AGAGTTAGTC |
| | TCTATCTGTC |
| 251 | AGGTTAAAGA TGAGCATGCT TTCTATAACG ATTTGTCTTT |
| | ATTACATATG |
| 301 | ACTCAGGCTG TGCCTTCGTA TTCTGCAACG TATGATTGTG |
| | CTGTAGTTTT |
| 351 | TGGCGGGCCT TTGCCAGCGC TACGTCAGCG CTTAGATTTT |
| | TTGGTGCGAG |
| 401 | AGTGGCAGCG TGGCGTGCGC TTTAAGAAAA TCGTTTTTCT |
| | ATGTGGAGAG |
| 451 | CGAGGGCGCT ATCAGTCTAT TGAAGAACAA GAGCATTTCT |
| | TTGATTCTCG |
| 501 | GTACAATCCT TTCCCTACTG AAGAGAACTG GGAATCTGGT |
| | AACCGAGTTA |
| 551 | CTCCCTCTTC TGAAGAAGAG ATTGCCAAAT TTGTTTGGAT |
| | GCAAATGCTT |
| 601 | TTACCTAGAG CATGGCGAGA TAGTACTTCA GGAGTCAGAG |
| | TGACATTTCT |
| 651 | TCTAGCAAAG CCAGAGGAAA ATCGTGTGGT TGCGAATCGT |
| | AAGGACACCT |
| 701 | TACTTTTATT CCGTTCTTAT CAAGAAGCGT TTCCGGGACG |
| | CGTGTTATTT |
| 751 | GTAAGTAGTC AACCCTTTAT CGGTTTAGAT GCTTGCAGGG |
| | TCGGGCAGTT |
| 801 | TTTCAAAGGG GAAAGCTATG ATCTTGCTGG ACCTGGATTT |
| | GCTCAAGGAG |
| 851 | TCTTGAAGTA TCATTGGGCT CCAAGGATTT GTCTACATAC |
| | TTTAGCGGAA |
| 901 | TGGTTAAAGG AAACGAACGG CTGCTTAAAT ATTTCAGAGG |
| | GTTGTTTTGG |
| 951 | ATGA |

The PSORT algorithm predicts a periplasmic location (0.894).

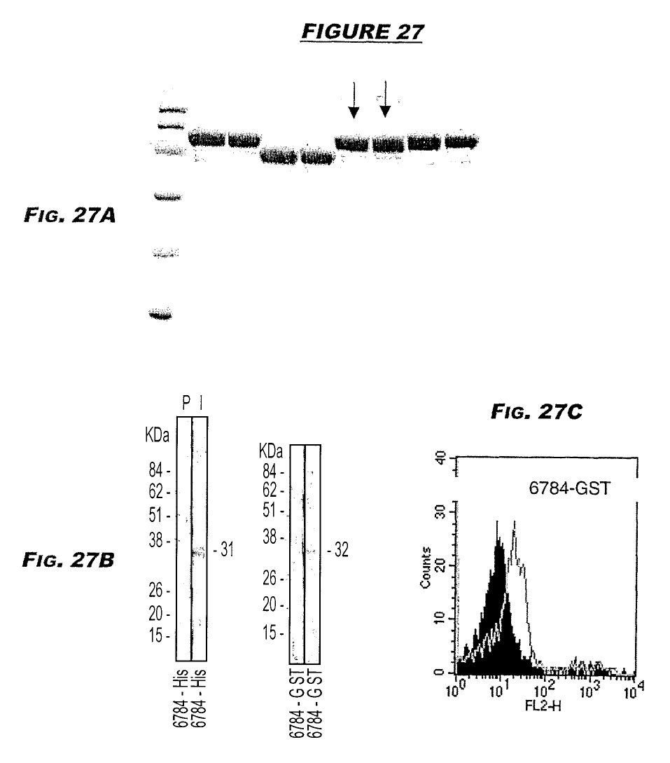

The protein was expressed in E. coli and purified as a his-tag product and as a GST-fusion product, as shown in FIG. 27A. The recombinant proteins were used to immunise mice, whose sera were used in a Western blot (FIG. 27B). The GST-fusion product was used for FACS analysis (FIG. 27C).

The cp6784 protein was also identified in the 2D-PAGE experiment (Cpn0498).

These experiments show that cp6784 is a surface-exposed and immunoaccessible protein, and that it is a useful immunogen. These properties are not evident from the sequence alone.

### Example 28

The following C. pneumoniae protein (PID 4376960) was expressed <SEQ ID 55; cp6960>:

| | |
|---|---|
| 1 | MNRRWNLVLA TVALALSVAS CDVRSKDKDK DQGSLVEYKD |
| | NKDTNDIELS |
| 51 | DNQKLSRTFG HLLARQLRKS EDMFFDIAEV AKGLQAELVC |
| | KSAPLTETEY |
| 101 | EEKMAEVQKL VFEKKSKENL SLAEKFLKEN SKNAGVVEVQ |
| | PSKLQYKIIK |
| 151 | EGAGKAISGK PSALLHYKGS FINGQVFSSS EGNNEPILLP |
| | LGQTIPGFAL |
| 201 | GMQGMKEGET RVLYIHPDLA YGTAGQLPPN SLLIFEINLI |
| | QASADEVAAV |
| 251 | RQEGNQGE* |

A predicted signal peptide is highlighted.

The cp6960 nucleotide sequence <SEQ ID 56> is:

| | |
|---|---|
| 1 | ATGAACAGAC GGTGGAATTT AGTTTTAGCA ACAGTAGCTC |
| | TGGCACTCTC |
| 51 | CGTCGCTTCT TGTGACGTAC GGTCTAAGGA TAAAGACAAG |
| | GATCAGGGGT |
| 101 | CGTTAGTGGA ATATAAAGAT AACAAAGATA CCAATGACAT |
| | AGAATTATCC |
| 151 | GATAATCAAA AGTTATCCAG AACATTTGGT CATTTATTAG |
| | CACGCCAATT |
| 201 | ACGCAAGTCA GAAGATATGT TTTTTGATAT TGCAGAAGTG |
| | GCTAAGGGGT |
| 251 | TGCAGGCGGA ATTGGTTTGT AAAAGTGCTC CTTTAACAGA |
| | AACAGAGTAT |
| 301 | GAAGAAAAAA TGGCTGAAGT ACAGAAGTTG GTTTTTGAAA |
| | AAAAATCAAA |
| 351 | AGAAAATCTT TCATTGGCAG AAAAATTCTT AAAAGAAAAT |
| | AGCAAGAACG |
| 401 | CTGGTGTTGT TGAAGTGCAA CCAAGTAAAT TGCAATACAA |
| | AATTATTAAA |
| 451 | GAAGGTGCAG GGAAAGCAAT TTCAGGTAAA CCTTCAGCTC |
| | TATTGCACTA |
| 501 | CAAGGGTTCC TTCATCAATG GCCAAGTATT TAGCAGTTCA |
| | GAAGGCAACA |
| 551 | ATGAGCCTAT CTTGCTTCCT CTAGGCCAAA CAGTTCCTGG |
| | TTTTGCTTTA |
| 601 | GGTATGCAGG GCATGAAAGA AGGAGAAACT CGAGTTCTCT |
| | ACATCCATCC |
| 651 | TGATCTTGCT TACGGAACCG CAGGACAACT TCCTCCAAGC |
| | TCTTTATTAA |
| 701 | TTTTTGAAAT TAACTTGATT CAGGCTTCAG CAGATGAAGT |
| | TGCTGCTGTA |
| 751 | CCCCAAGAAG GAAATCAAGG TGAATGA |

The PSORT algorithm predicts periplasmic space location (0.930).

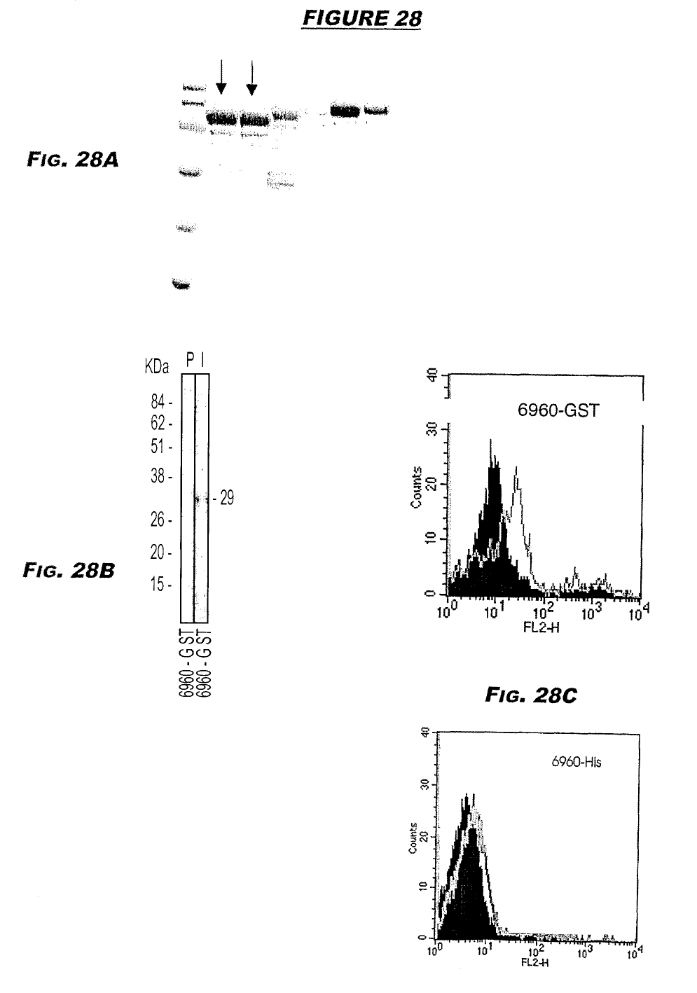

The protein was expressed in E. coli and purified as a his-tag product and as a GST-fusion product, as shown in FIG. 28A. The recombinant proteins were used to immunise mice, whose sera were used in a Western blot (FIG. 28B) and for FACS analysis (FIG. 28C).

The cp6960 protein was also identified in the 2D-PAGE experiment.

These experiments show that cp6960 is a surface-exposed and immunoaccessible protein, and that it is a useful immunogen. These properties are not evident from the sequence alone.

### Example 29

The following C. pneumoniae protein (PID 4376968) was expressed <SEQ ID 57; cp6968>:

| | |
|---|---|
| 1 | MKFLLYVPLL LVLVSTGCDA KPVSFEPFSG KLSTQRFEPQ |
| | HSAEEYFSQG |
| 51 | QEFLKKGNFR KALLCFGIIT HHFPRDILRN QAQYLIGVCY |
| | FTQDHPDLAD |
| 101 | KAFASYLQLP DAEYSEELFQ MKYAIAQRFA QGKRKRICRL |
| | EGFPKLMNAD |
| 151 | EDALRIYDEI LTAFPSKDLG AQALYSKAAL LIVKNDLTEA |
| | TKTLKKLTLQ |
| 201 | FPLHILSSEA FVRLSEIYLQ QAKKEPHNLQ YLHFAKLNEE |
| | AMKKQHPNHP |
| 251 | LNEVVSANVG AMREHYARGL YATGRFYEKK KKAEAANIYY |
| | RTAITNYPDT |
| 301 | LLVAKCQKRL DRISKHTS* |

A predicted signal peptide is highlighted.

The cp6968 nucleotide sequence <SEQ ID 58> is:

| | |
|---|---|
| 1 | ATGAAATTTC TATTATACGT TCCACTTCTT CTTGTTCTCG |
| | TATCTACGGG |
| 51 | GTGCGATGCA AAACCTGTTT CTTTTGAGCC CTTTTCAGGA |
| | AAGCTTTCCA |
| 101 | CCCAGCGTTT TGAGCCTCAG CACTCTGCTG AAGAATATTT |
| | TTCTCAGGGA |
| 151 | CAGGAATTCT TAAAAAAAGG AAATTTCAGA AAAGCTTTAC |
| | TATGCTTTGG |
| 201 | AATCATTACG CATCACTTCC CTAGGGACAT CTTGCGTAAT |
| | CAAGCACAGT |
| 251 | ATCTTATAGG AGTCTGTTAC TTCACGCAGG ATCACCCAGA |
| | TTTAGCAGAC |
| 301 | AAGGCATTTG CATCTTACTT ACAACTTCCT GATGCGGAGT |
| | ACTCTGAAGA |
| 351 | GTTGTTCCAG ATGAAATATG CGATTGCTCA AAGATTTGCT |
| | CAAGGGAAGC |
| 401 | GTAAACGGAT TTGTCGATTA GAGGGCTTCC CAAAACTAAT |
| | GAATGCTGAT |
| 451 | GAAGATGCGC TACGCATTTA TGACGAGATT CTAACAGCGT |
| | TTCCTAGTAA |
| 501 | AGACTTAGGA GCTCAGGCCC TCTATAGTAA AGCTGCGTTA |
| | CTTATTGTAA |
| 551 | AAAACGATCT TACAGAAGCC ACCAAAACCT TAAAAAAACT |
| | CACGTTACAA |
| 601 | TTTCCTCTAC ATATTTTATC TTCAGAGGCC TTTGTACGTT |
| | TATCGGAAAT |
| 651 | CTATTTACAG CAAGCTAAGA AAGAGCCTCA CAATCTTCAA |
| | TATCTTCATT |
| 701 | TTGCAAAGCT TAATGAAGAG GCAATGAAAA AGCAGCATCC |
| | TAACCATCCT |
| 751 | CTGAATGAGG TTGTTTCTGC TAATGTTGGA GCTATGCGGG |
| | AACATTATGC |
| 801 | TCGAGGTTTG TATGCCACAG GTCGTTTCTA TGAGAAGAAG |
| | AAAAAGCCCG |
| 851 | AGGCTGCGAA TATCTATTAC CGCACTGCGA TTACAAACTA |
| | CCCAGACACT |
| 901 | TTATTAGTGG CTAAATGTCA AAAGCGTCTA GATAGAATAT |
| | CTAAGCATAC |
| 951 | TTCCTAA |

The PSORT algorithm predicts an inner membrane location (0.790).

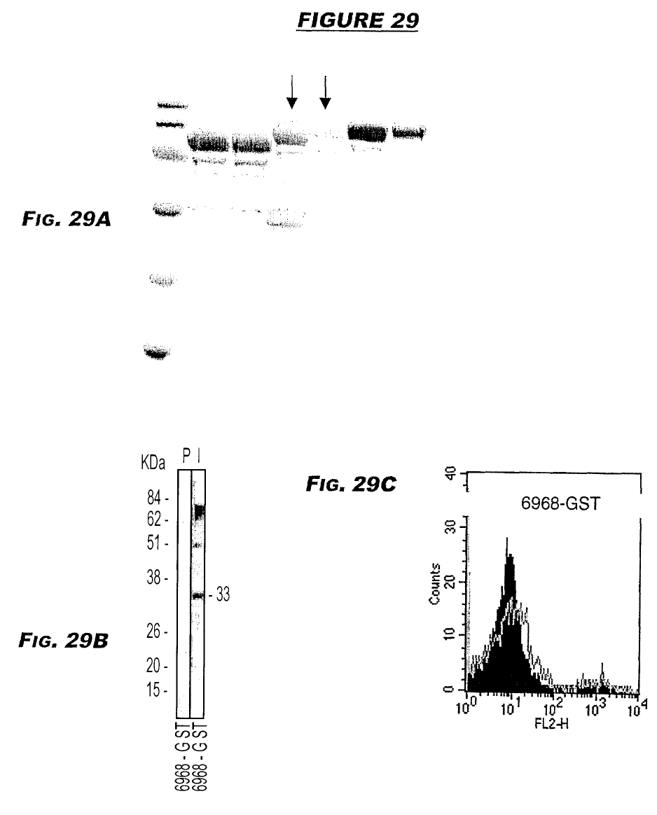

The protein was expressed in E. coli and purified as a his-tag product and as a GST-fusion product, as shown in FIG. 29A. The recombinant GST-fusion was used to immunise mice, whose sera were used in a Western blot (FIG. 29B) and for FACS analysis (FIG. 29C).

This protein also showed good cross-reactivity with human sera, including sera from patients with pneumonitis.

These experiments show that cp6968 is a surface-exposed and immunoaccessible protein, and that it is a useful immunogen. These properties are not evident from the sequence alone.

### Example 30

The following C. pneumoniae protein (PID 4376998) was expressed <SEQ ID 59; cp6998>:

| | |
|---|---|
| 1 | MKKLLKSALL SAAFAGSVGS LQALPVGNPS DPSLLIDGTI |
| | WEGAAGDPCD |
| 51 | PCATWCDAIS IRAGFYGDYV FDRILKVDAP KTFSMGAKPT |
| | GSAAANYTTA |
| 101 | VDRPNPAYNK HLHDAEWFTN AGFIALNIWD RFDVFCTLGA |
| | SNGYIRGNST |
| 151 | AFNLVGLFGV KGTTVNANEL PNVSLSNGVV ELYTDTSFSW |
| | SVGARGALWE |
| 201 | CGCATLGAEF QYAQSKPKVE ELNVICNVSQ SFVNKPKGYK |
| | GVAFPLPTDA |
| 251 | GVATATGTKS ATINYHEWQV GASLSYRLNS LVPYIGVQWS |
| | RATFDADNIR |
| 301 | IAQPKLPTAV LNLTAWNPSL LGNATALSTT DSFSDFMQIV |
| | SCQINKFKSR |
| 351 | KACGVTVGAT LVDADKWSLT AEARLINERA AHVSGQFRF* |

A predicted signal peptide is highlighted.

The cp6998 nucleotide sequence <SEQ ID 60> is:

| | |
|---|---|
| 1 | ATGAAAAAAC TCTTAAAGTC GGCGTTATTA TCCGCCGCAT |
| | TTGCTGGTTC |
| 51 | TGTTGGCTCC TTACAAGCCT TGCCTGTAGG GAACCCTTCT |
| | GATCCAAGCT |
| 101 | TATTAATTGA TGGTACAATA TGGGAAGGTG CTGCAGGAGA |
| | TCCTTGCGAT |
| 151 | CCTTGCGCTA CTTGGTGCGA CGCTATTAGC TTACGTGCTG |
| | GATTTTACGG |
| 201 | AGACTATGTT TTCGACCGTA TCTTAAAAGT AGATGCACCT |
| | AAAACATTTT |
| 251 | CTATGGGAGC CAAGCCTACT GGATCCGCTG CTGCAAACTA |
| | TACTACTGCC |
| 301 | GTAGATAGAC CTAACCCGGC CTACAATAAG CATTTACACG |
| | ATGCAGAGTG |
| 351 | GTTCACTAAT GCAGGCTTCA TTGCCTTAAA CATTTGGGAT |
| | CGCTTTGATG |
| 401 | TTTTCTGTAC TTTAGGAGCT TCTAATGGTT ACATTAGAGG |
| | AAACTCTACA |
| 451 | GCGTTCAATC TCGTTGGTTT ATTCGGAGTT AAAGGTACTA |
| | CTGTAAATGC |
| 501 | AAATGAACTA CCAAACGTTT CTTTAAGTAA CGGAGTTGTT |
| | GAACTTTACA |
| 551 | CAGACACCTC TTTCTCTTGG AGCGTAGGCG CTCGTGGAGC |
| | CTTATGGGAA |
| 601 | TGCGGTTGTG CAACTTTGGG AGCTGAATTC CAATATGCAC |
| | AGTCCAAACC |
| 651 | TAAAGTTGAA GAACTTAATG TGATCTGTAA CGTATCGCAA |
| | TTCTCTGTAA |
| 701 | ACAAACCCAA GGGCTATAAA GGCGTTGCTT TCCCCTTGCC |
| | AACAGACGCT |
| 751 | GGCGTAGCAA CAGCTACTGG AACAAAGTCT GCGACCATCA |
| | ATTATCATGA |
| 801 | ATGGCAAGTA GGAGCCTCTC TATCTTACAG ACTAAACTCT |
| | TTAGTGCCAT |
| 851 | ACATTGGAGT ACAATGGTCT CGAGCAACTT TTGATGCTGA |
| | TAACATCCGC |
| 901 | ATTGCTCAGC CAAAACTACC TACAGCTGTT TTAAACTTAA |
| | CTGCATGGAA |
| 951 | CCCTTCTTTA CTAGGAAATG CCACAGCATT GTCTACTACT |
| | GATTCGTTCT |
| 1001 | CAGACTTCAT GCAAATTGTT TCCTGTCAGA TCAACAAGTT |
| | TAAATCTAGA |
| 1051 | AAAGCTTGTG GAGTTACTGT AGGAGCTACT TTAGTTGATG |
| | CTGATAAATG |
| 1101 | GTCACTTACT GCAGAAGCTC GTTTAATTAA CGAGAGAGCT |
| | GCTCACGTAT |
| 1151 | CTGGTCAGTT CAGATTCTAA |

The PSORT algorithm predicts an outer membrane location (0.707).

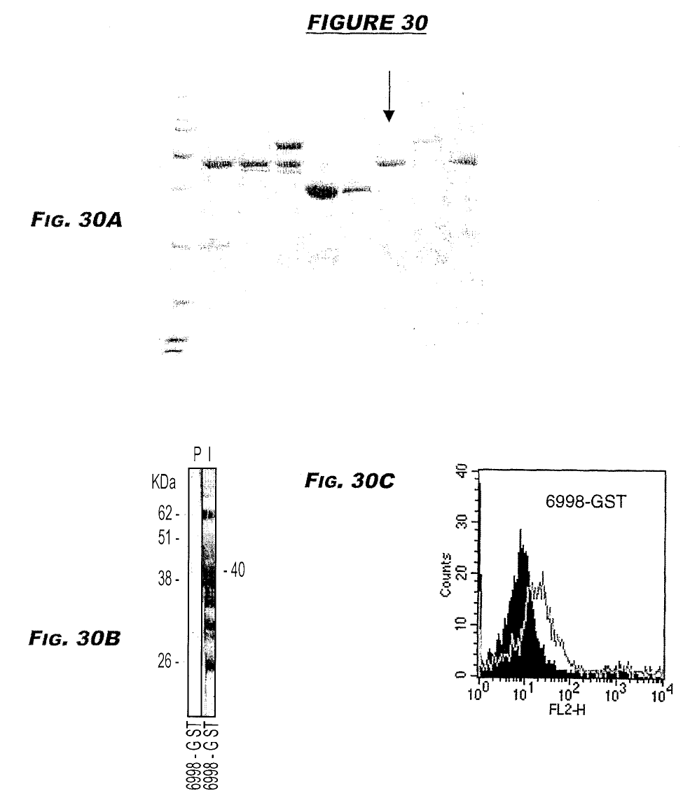

The protein was expressed in E. coli and purified as a GST-fusion (FIG. 30A) and as a his-tag product. The recombinant GST-fusion protein was used to immunise mice, whose sera were used in a Western blot (FIG. 30B) and for FACS analysis (FIG. 30C).

The cp6998 protein was also identified in the 2D-PAGE experiment (Cpn0695) and showed good cross-reactivity with human sera, including sera from patients with pneumonitis.

These experiments show that cp6998 is a surface-exposed and immunoaccessible protein, and that it is a useful immunogen. These properties are not evident from the sequence alone.

### Example 31

The following C. pneumoniae protein (PID 4377102) was expressed <SEQ ID 61; cp7102>:

| | |
|---|---|
| 1 | MKHTFTKRVL FFFFLVIPIP LLLNLMVVGF FSFSAAKANL |
| | VQVLHTRATN |
| 51 | LSIEFEKKLT IHKLFLDRLA NTLALKSYAS PSAEPYAQAY |
| | NEMMALSNTD |
| 101 | FSLCLIDPFD GSVRTKNPGD PFIRYLKQHP EMKKKLSAAV |
| | GKAFLLTIPG |
| 151 | KPLLHYLILV EDVASWDSTT TSGLLVSFYP MSFLQKDLFQ |
| | SLHITKGNIC |
| 201 | LVNKYGEVLF CAQDSESSFV FSLDLPNLPQ FQARSPSAIE |
| | IEKASGILGG |
| 251 | ENLITVSINK KRYLGLVLNK IPIQGTYTLS LVPVSDLIQS |
| | ALKVPLNICF |
| 301 | FYVLAFLLMW WIFSKINTKL NKPLQELTFC MEAAWRGNHN |
| | VRFEPQPYGY |
| 351 | EFNELGNIFN CTLLLLLNSI EKADIDYHSG EKLQKELGIL |
| | SSLQSALLSP |
| 401 | DFPTFPKVTF SSQHLRRRQL SGHFNGWTVQ DGGDTLLGII |
| | GLAGDIGLPS |
| 451 | YLYALSARSL FLAYASSDVS LQKISKDTAD SFSKTTEGNE |
| | AVVAMTFIKY |
| 501 | VEKDRSLELL SLSEGAPTMF LQRGESFVRL PLETHQALQP |
| | GDRLICLTGG |
| 551 | EDILKYFSQL PIEELLKDPL NPLNTENLID SLTMMLNNET |
| | EHSADGTLTI |
| 601 | LSFS* |

A predicted signal peptide is highlighted.

The cp7102 nucleotide sequence <SEQ ID 62> is:

| | |
|---|---|
| 1 | ATGAAACATA CCTTTACCAA GCGTGTTCTA TTTTTTTTCT |
| | TTTTAGTGAT |
| 51 | TCCCATTCCC CTACTCCTCA ATCTTATGGT CGTAGGTTTT |
| | TTCTCATTTT |
| 101 | CTGCCGCTAA AGCAAATTTA GTACAGGTCC TCCATACCCG |
| | TGCTACGAAC |
| 151 | TTAAGTATAG AATTCGAAAA AAAACTGACG ATACACAAGC |
| | TTTTCCTCGA |
| 201 | TAGACTTGCC AACACATTAG CCTTAAAATC CTATGCATCT |
| | CCTTCTGCAG |
| 251 | AGCCCTATGC ACAGGCATAC AATGAGATGA TGGCACTCTC |
| | CAATACAGAC |
| 301 | TTTTCCTTAT GCCTTATAGA TCCCTTTGAT GGATCTGTAA |
| | GGACGAAAAA |
| 351 | TCCTGGAGAC CCTTTCATTC GCTATCTAAA ACAGCATCCT |
| | GAAATGAAGA |
| 401 | AAAAGCTATC CGCAGCTGTA GGGAAAGCCT TTTTATTGAC |
| | CATTCCAGGT |
| 451 | AAACCACTTT TACATTATCT TATTCTAGTT GAAGATGTCG |
| | CATCTTGGGA |
| 501 | TTCTACAACG ACTTCAGGAC TGCTTGTAAG TTTCTATCCC |
| | ATGTCTTTTT |
| 551 | TACAGAAAGA TTTATTCCAA TCCTTACACA TCACCAAAGG |
| | AAATATCTGC |
| 601 | CTTGTAAATA AGTATGGCGA GGTCCTCTTC TGTGCTCAGG |
| | ACAGTGAATC |
| 651 | TTCTTTTGTA TTTTCTCTAG ATCTCCCTAA TTTACCGCAA |
| | TTCCAAGCAA |
| 701 | GAAGCCCCTC TGCCATAGAA ATTGAGAAAG CTTCTGGAAT |
| | TCTTGGTGGG |
| 751 | GAGAACCTAA TCACAGTGAG TATCAACAAG AAACGCTACC |
| | TAGGATTGGT |
| 801 | ACTGAATAAA ATTCCTATCC AAGGGACCTA CACTCTATCT |
| | TTAGTTCCAG |
| 851 | TTTCTGATCT CATCCAATCC GCCTTGAAAG TTCCTCTCAA |
| | TATTTGTTTT |
| 901 | TTCTATGTAC TTGCTTTCCT CCTCATGTGG TGGATTTTCT |
| | CTAAGATCAA |
| 951 | CACCAAACTT AACAAGCCTC TTCAAGAACT GACCTTCTGT |
| | ATGGAAGCTG |
| 1001 | CCTGGCGAGG AAACCATAAC GTGAGGTTTG AACCCCAGCC |
| | TTACGGTTAT |
| 1051 | GAATTCAATG AACTAGGAAA TATTTTCAAT TGCACTCTCC |
| | TACTCTTATT |
| 1101 | GAATTCCATT GAGAAAGCAG ATATCGATTA CCATTCAGGC |
| | GAAAAATTAC |
| 1151 | AAAAAGAATT AGGGATTTTA TCTTCACTAC AAAGTGCGTT |
| | ACTAAGTCCG |
| 1201 | GATTTCCCTA CGTTCCCTAA AGTTACCTTT AGTTCCCAAC |
| | ATCTCCGGAG |
| 1251 | AAGGCAACTT TCCGGTCATT TTAATGGTTG GACAGTTCAA |
| | GATGGTGGCG |
| 1301 | ATACCCTTTT AGGGATCATA GGGCTCGCTG GCGATATTGG |
| | TCTTCCTTCC |
| 1351 | TATCTCTATG CTTTATCCGC ACGGAGTCTT TTTCTTGCCT |
| | ATGCTTCCTC |
| 1401 | GGACGTTTCG TTACAAAAAA TCAGCAAGGA TACTGCCGAC |
| | AGCTTCTCAA |
| 1451 | AAACAACAGA AGGCAATGAG GCTGTAGTTG CTATGACTTT |
| | CATTAAATAT |
| 1501 | GTAGAAAAAG ATCGATCTCT AGAGCTCCTC TCGTTAAGCG |
| | AGGGAGCTCC |
| 1551 | TACCATGTTT CTACAACGAG GAGAATCTTT CGTACGTCTC |
| | CCCTTAGAGA |
| 1601 | CTCACCAAGC TCTACAGCCT GGAGATCGGT TGATCTGCCT |
| | CACTGGAGGA |
| 1651 | GAAGACATCC TCAAGTACTT TTCTCAGCTT CCTATTGAAG |
| | AGCTCTTAAA |
| 1701 | AGATCCTTTA AACCCTCTAA ATACAGAGAA TCTTATTGAT |
| | TCTCTAACCA |
| 1751 | TGATGTTAAA CAACGAAACC GAACATTGTG CAGATGGAAC |
| | TCTGACCATC |
| 1801 | CTTTCATTT CATAA |

The PSORT algorithm predicts an inner membrane location (0.338).

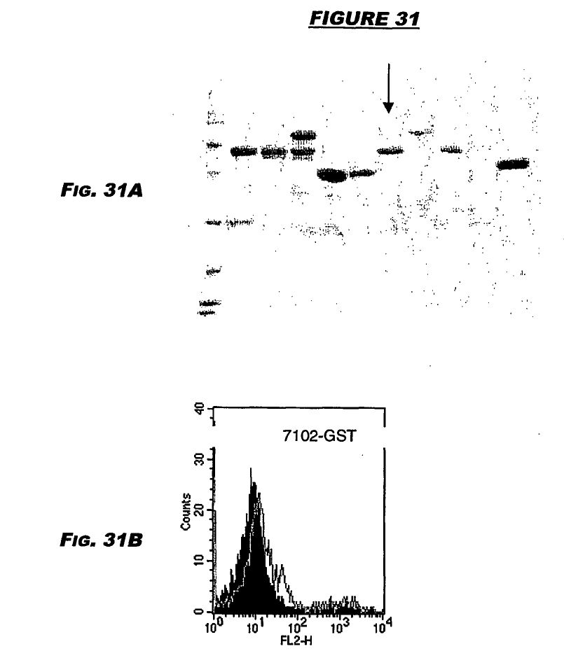

The protein was expressed in E. coli and purified as a his-tag product and as a GST-fusion product. The purified GST-fusion product is shown in FIG. 31A. The recombinant GST-fusion protein was used to immunise mice, whose sera were used in a Western blot and for FACS analysis (FIG. 31B).

These experiments show that cp7102 is a surface-exposed and immunoaccessible protein, and that it is a useful immunogen. These properties are not evident from the sequence alone.

### Example 32

The following C. pneumoniae protein (PID 4377106) was expressed <SEQ ID 63; cp7106>:

| | |
|---|---|
| 1 | MKDLGTLGGT SSTAKTVSPD GKVIMGRSQI ADGSWHAFMC |
| | HTDFSSNNVL |
| 51 | FDLDNTYKTL RENGRQLNSI FNLQNMMLQR ASDHEFTEFG |
| | RSNIALGAGL |
| 101 | YVNALQNLPS NLAAQYFGIA YKIRPKYRLG VFLDHNFSSH |
| | VPNNFNVSHN |
| 151 | RLWMGAFIGW CDSDALGSSV KVSFGYGKQK ATITREQLEN |
| | TEAGSGESHF |
| 201 | EGVAAQIEGR YGKSLGGHVR VQPFLGLQFV HITRKEYTEN |
| | AVQFPVHYDP |
| 251 | IDYSTGVVYL GIGSHIALVD SLHVGTRMGM EQNFAAHTDR |
| | FSGSIASIGN |
| 301 | FVFEKLDVTH TRAFAEMRVN YELPYLQSLN LILRVNQQPL |
| | QGVMGFSSDL |
| 351 | RYALGF* |

The cp7106 nucleotide sequence <SEQ ID 64> is:

| | |
|---|---|
| 1 | ATGAAAGATT TGGGGACTCT TGGGGGTACC TCTTCTACAG |
| | CAAAAACAGT |
| 51 | GTCCCCAGAT GGTAAAGTGA TCATGGGTAG ATCACAAATT |
| | GCTGATGGCA |
| 101 | GTTGGCACGC ATTTATGTGT CATACGGATT TCTCCTCTAA |
| | TAATGTACTC |
| 151 | TTTGATCTCG ATAATACGTA TAAAACTCTA AGAGAAAATG |
| | GCCGTCAGCT |
| 201 | AAATTCCATA TTCAACCTAC AAAATATGAT GTTACAGAGA |
| | GCCTCAGATC |
| 251 | ATGAGTTCAC AGAGTTTGGA AGGAGTAACA TCGCTCTTGG |
| | TGCCGGGCTT |
| 301 | TATGTGAATG CCTTGCAGAA TCTCCCTAGC AATTTAGCAG |
| | CACAATATTT |
| 351 | TGGAATCGCA TACAAAATAC GTCCTAAATA TCGTTTGGGG |
| | GTGTTTTTGG |
| 401 | ACCATAATTT CAGCTCCCAC GTTCCTAATA ATTTTAACGT |
| | AAGCCACAAT |
| 451 | AGACTCTGGA TGGGAGCCTT TATTGGATGG CAGGATTCTG |
| | ATGCTCTAGG |
| 501 | ATCTAGTGTC AAGGTGTCTT TCGGATATGG AAAACAAAAA |
| | GCCACGATTA |
| 551 | CAAGAGAGCA ATTAGAGAAT ACAGAAGCCG GGAGTGGGGA |
| | GAGCCATTTT |
| 601 | GAAGGGGTCG CTGCTCAGAT AGAAGGGCGG TATGGTAAGA |
| | GCCTCGGAGG |
| 651 | ACATGTCAGG GTCCAGCCTT TCCTAGGACT GCAGTTTGTC |
| | CACATTACAA |
| 701 | GGAAAGAATA TACCGAAAAT GCAGTGCAAT TTCCTGTACA |
| | CTATGATCCT |
| 751 | ATAGACTATT CTACAGGTGT AGTGTATTTA GGAATTGGAT |
| | CTCATATTGC |
| 801 | ACTTGTAGAT TCTTTACATG TAGGCACACG CATGGGAATG |
| | GAGCAAAACT |
| 851 | TTGCAGCCCA TACGGACAGG TTCTCAGGAT CTATAGCGTC |
| | TATTGGAAAC |
| 901 | TTTGTGTTTG AAAAGCTTGA TGTGACTCAC ACAAGGGCAT |
| | TTGCGGAAAT |
| 951 | GCGTGTCAAC TATGAGCTTC CCTATCTACA GTCTCTGAAT |
| | CTTATTCTAC |
| 1001 | GAGTTAATCA ACAGCCCCTA CAAGGGGTTA TGGGATTTTC |
| | CAGTGATCTT |
| 1051 | AGGTATGCCT TAGGATTCTA A |

The PSORT algorithm predicts a cytoplasmic location (0.224).

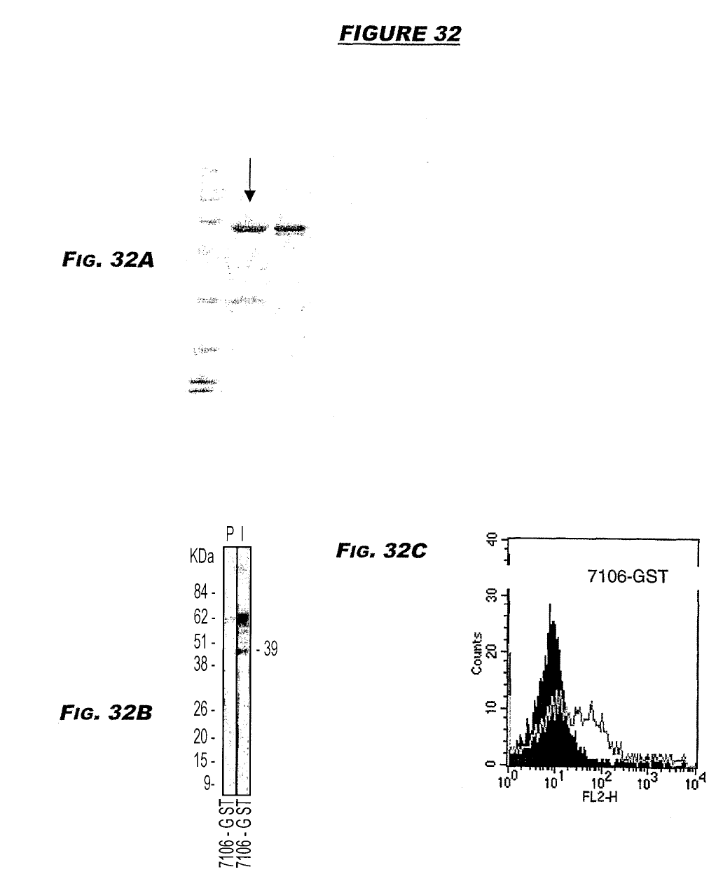

The protein was expressed in E. coli and purified as a his-tag product and as a GST-fusion product. The purified GST-fusion product is shown in FIG. 32A. The recombinant GST-fusion protein was used to immunise mice, whose sera were used in a Western blot (FIG. 32B) and for FACS analysis (FIG. 32C).

This protein also showed very good cross-reactivity with human sera, including sera from patients with pneumonitis.

These experiments show that cp7106 is a surface-exposed and immunoaccessible protein, and that it is a useful immunogen. These properties are not evident from the sequence alone.

### Example 33

The following C. pneumoniae protein (PID 4377228) was expressed <SEQ ID 65; cp7228>:

| | |
|---|---|
| 1 | MTAVLILTSF PSEESARSLA RHLITERLAS CVHVFPKGTS |
| | TYLWFGKLCE |
| 51 | SEEHHIQIKS IDIRFSEICL AIQEFSGYEV PEVLLFPIEN |
| | GDPRYLNWLT |
| 101 | ILSYPEKPPL SD* |

The cp7228 nucleotide sequence <SEQ ID 66> is:

| | |
|---|---|
| 1 | ATGACTGCTG TTCTTATTCT TACATCTTTC CCTTCGGAGG |
| | AAAGTGCTCG |
| 51 | CTCCTTAGCT AGACATCTGA TTACAGAGCG TCTTGCTTCC |
| | TGTGTGCATG |
| 101 | TATTCCCTAA AGGCACATCG ACATATCTAT GGGAAGGCAA |
| | GCTATGTGAG |
| 151 | TCTGAAGAAC ATCATATACA AATCAAATCG ATAGACATAC |
| | GCTTCTCGGA |
| 201 | AATTTGTCTT GCTATTCAGG AGTTCTCTGG CTATGAGGTT |
| | CCTGAAGTCT |
| 251 | TACTATTTCC TATTGAAAAT GGGGATCCGA GGTACTTGAA |
| | TTGGTTAACG |
| 301 | ATTCTCAGCT ATCCAGAGAA GCCTCCGCTT TCAGATTAG |

The PSORT algorithm predicts an inner membrane location (0.040).

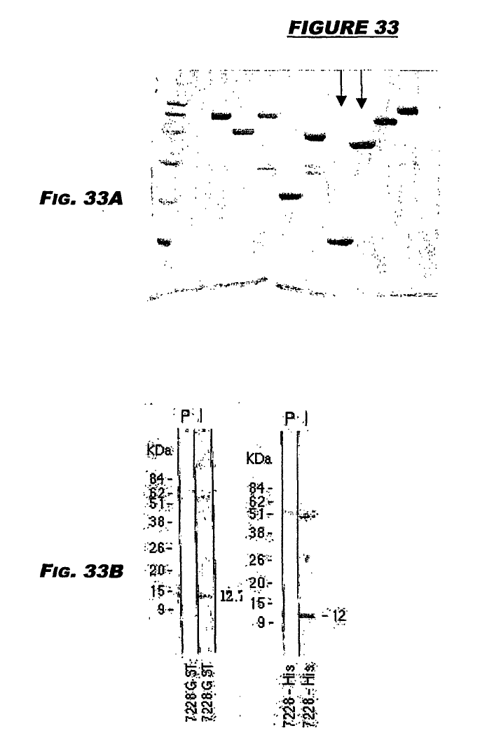

The protein was expressed in E. coli and purified as a his-tag product and as a GST-fusion product, as shown in FIG. 33A (his-tag=left-hand arrow, GST=right-hand arrow). The proteins were used to immunise mice, whose sera were used in a Western blot (FIG. 33B) and FACS analysis.

These experiments show that cp7228 is a surface-exposed and immunoaccessible protein, and that it is a useful immunogen. These properties are not evident from the sequence alone.

### Example 34

The following C. pneumoniae protein (PID 4377170) was expressed <SEQ ID 67; cp7170>:

| | |
|---|---|
| 1 | MNSKMLKHLR LATLSFSMFF GIVSSPAVYA LGAGNPAAPV |
| | LPGVNPEQTG |
| 51 | WCAFQLCNSY DLFAALAGSL KFGFYGDYVF SESAHITNVP |
| | VITSVTTSGT |
| 101 | GTTPTITSTT KNVDFDLNNS SISSSCVFAT IALQETSPAA |
| | IPLLDIAFTA |
| 151 | RVGGLKQYYR LPLNAYRDFT SNPLNAESEV TDGLIEVQSD |
| | YGIVWGLSLQ |
| 201 | KVLWKDGVSF VGVSADYRHG SSPINYIIVY NKANPEIYFD |
| | ATDGNLSYKE |
| 251 | WSASIGISTY LNDYVLPYAS VSIGNTSRKA PSDSFTELEK |
| | QFTNFKFKIR |
| 301 | KITNFDRVNF CFGTTCCISN NFYYSVEGRW GYQRAINITS |
| | GLQF* |

A predicted signal peptide is highlighted.

The cp7170 nucleotide sequence <SEQ ID 68> is:

| | |
|---|---|
| 1 | ATGAATAGCA AGATGCTAAA ACATTTACGT TTAGCAACCC |
| | TTTCCTTCTC |
| 51 | TATGTTCTTC GGGATTGTAT CTTCTCCCGC AGTATATGCC |
| | CTAGGGGCTG |
| 101 | GAAACCCTGC AGCTCCAGTA CTCCCAGGTG TGAATCCTGA |
| | GCAAACGGGA |
| 151 | TGGTGTGCCT TCCAACTTTG TAATAGTTAC GATCTTTTTG |
| | CTGCTCTTGC |
| 201 | AGGAAGCCTC AAATTTGGGT TCTATGGAGA TTATGTCTTC |
| | TCAGAAAGTG |
| 251 | CCCATATTAC CAATGTCCCT GTCATTACCT CCGTTACGAC |
| | TTCAGGCACA |
| 301 | GGAACAACGC CAACCATTAC CTCTACAACT AAAAACGTAG |
| | ACTTTGATCT |
| 351 | TAACAACAGC TCCATCAGCT CGAGCTGTGT TTTTGCAACC |
| | ATAGCTCTAC |
| 401 | AGGAAACATC CCCAGCTGCC ATTCCCCTTT TAGATATAGC |
| | CTTCACTGCA |
| 451 | CGTGTCGGAG GACTTAAGCA GTACTACCGC CTCCCTCTCA |
| | ATGCTTACAG |
| 501 | AGACTTCACT TCAAATCCTT TAAATGCAGA ATCTGAAGTT |
| | ACAGATGGTC |
| 551 | TCATTGAAGT CCAGTCAGAC TATGGAATTG TCTGGGGTCT |
| | GAGTTTACAA |
| 601 | AAAGTATTGT GGAAAGATGG AGTGTCTTTT GTAGGGGTGA |
| | GCGCTGACTA |
| 651 | CCGTCACGGT TCCAGTCCCA TCAACTATAT CATCGTTTAC |
| | AACAAGGCCA |
| 701 | ACCCCGAGAT CTATTTCGAT GCTACTGATG GAAACCTAAG |
| | CTATAAAGAA |
| 751 | TGGTCTGCAA GCATCGGCAT CTCTACGTAT CTTAATGACT |
| | ATGTGCTTCC |
| 801 | CTATGCATCC GTATCTATAG GAAATACTTC AAGAAAAGCT |
| | CCTTCTGATA |
| 851 | GCTTCACAGA ACTCGAAAAG CAATTTACGA ATTTTAAATT |
| | TAAAATTCGT |
| 901 | AAAATCACAA ACTTCGACAG AGTAAACTTC TGCTTCGGAA |
| | CTACCTGCTG |
| 951 | CATCTCAAAT AACTTCTACT ATAGTGTAGA AGGCCGTTGG |
| | GGATATCAGC |
| 1001 | GTGCTATCAA CATTACGTCA GGTCTGCAGT TTTAG |

The PSORT algorithm predicts a bacterial outer membrane location (0.936).

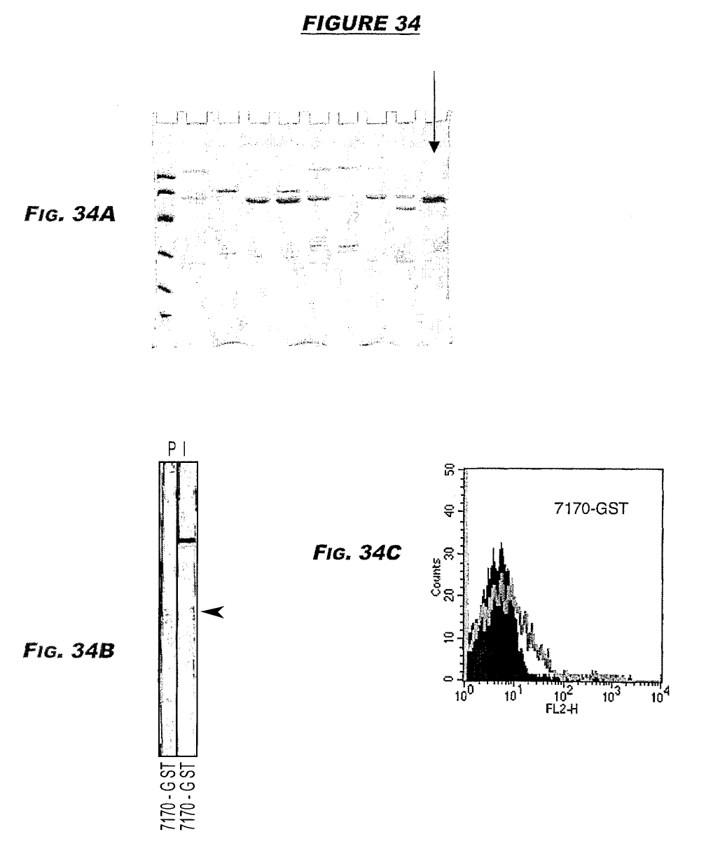

The protein was expressed in E. coli and purified as a his-tag product and as a GST-fusion product. The purified GST-fusion product is shown in FIG. 34A. The GST-fusion protein was used to immunise mice, whose sera were used in a Western blot (34B) and for FACS analysis (34C).

The cp7170 protein was also identified in the 2D-PAGE experiment (Cpn0854).

These experiments show that cp7170 is a surface-exposed and immunoaccessible protein, and that it is a useful immunogen. These properties are not evident from the sequence alone.

### Example 35

The following C. pneumoniae protein (PID 4377072) was expressed <SEQ ID 69; cp7072>:

| | |
|---|---|
| 1 | MDIKKLFCLF LCSSLIAMSP IYGKTGDYEK LTLTGINIID |
| | RNGLSETICS |
| 51 | KEKLKKYTKV DFLAPQPYQK VMRMYKNKRG DNVSCLTAYH |
| | TNGQIKQYLE |
| 101 | CLNNRAYGRY REWHVNGNIK IQAEVIGGIA DLHPSAESGW |
| | LFDQTTFAYN |
| 151 | DEGILEAAIV YEKGLLEGSS VYYHTNGNIW KECPYHKGVP |
| | QGKELTYTSS |
| 201 | GKLLKEQNYQ QGKRHGLSIR YSEDSEEDVL AWEEYHEGRL |
| | LKAEYLDPQT |
| 251 | HEIYATIHEG NGIQAIYGKY AVIETRAFYR GEPYGKVTRF |
| | DNSGTQIVQT |
| 301 | YNLLQGAKHG EEFFFYPETG KPKLLLNWHE GILNGIVKTW |
| | YPGGTLESCK |
| 351 | ELVNNKKSGL LTIYYPEGQI MATEEYDNDL LIKGEYFRPG |
| | DRHPYSKIDR |
| 401 | GCGTAVFFSS AGTITKKIPY QDGKPLLN* |

A predicted signal peptide is highlighted.

The cp7072 nucleotide sequence <SEQ ID 70> is:

| | |
|---|---|
| 1 | ATGGATATAA AAAAACTCTT TTGCTTATTT CTATGTTCTT |
| | CTCTAATTGC |
| 51 | CATGAGTCCC ATTTATGGGA AAACAGGTGA CTATGAGAAA |
| | CTCACCCTTA |
| 101 | CAGGGATCAA TATCATTGAT AGAAACGGCC TGTCAGAAAC |
| | TATTTGCTCT |
| 151 | AAAGAGAAGC TAAAGAAATA CACCAAGGTA GACTTTCTTG |
| | CTCCCCAGCC |
| 201 | CTATCAAAAG GTCATGAGGA TGTATAAAAA CAAACGCGGA |
| | GATAACGTTT |
| 251 | CTTGTTTAAC AGCCTATCAC ACTAACGGGC AAATTAAGCA |
| | GTACCTGGAG |
| 301 | TGTCTCAATA ATCGTGCTTA TGGAAGATAT CTGTAATGGC |
| | ACGTCAACGG |
| 351 | GAATATCAAA ATCCAAGCTG AGGTTATCGG AGGTATTGCG |
| | GATCTTCATC |
| 401 | CCTCAGCAGA GTCTGGCTGG CTATTTGATC AAACTACATT |
| | TGCCTATAAT |
| 451 | GATGAAGGTA TCTTAGAAGC CGCTATCGTC GATGAAAAAG |
| | GGCTGCTCGA |
| 501 | AGGATCTTCG GTGTATTACC ATACTAATGG GAATATTTGG |
| | AAAGAGTGTC |
| 551 | CCTATCATAA GGGAGTTCCT CAAGGTAAAT TCCTGACATA |
| | CACATCTTCG |
| 601 | GGGAAACTGC TCAAAGAACA GAATTACCAA CAAGGCAAAA |
| | GACACGGTCT |
| 651 | TTCGATTCGC TACAGCGAAG ATTCCGAAGA AGATGTTTTA |
| | GCCTGGGAAG |
| 701 | AATATCATGA GGGACGACTC CTAAAAGCAG AGTACTTAGA |
| | TCCTCAAACT |
| 751 | CACGAAATCT ATGCGACTAT ACACGAAGGG AACGGCATTC |
| | AAGCAATCTA |
| 801 | CGGCAAGTAT GCCGTTATAG AAACTAGGGC ATTTTACCGA |
| | GGGGAACCTT |
| 851 | ATGGAAAAGT TACCAGATTC GACAACTCCG GAACACAGAT |
| | TGTCCAAACG |
| 901 | TATAACCTTT TCGAAGGCGC GAAGCACGGA GAAGAATTTT |
| | TCTTTTATCC |
| 951 | TGAGACAGGG AAACCCAAGC TGCTTCTTAA TTGGCATGAA |
| | GGAATTTTAA |
| 1001 | ATGGGATAGT AAAAACTTGG TATCCCGGAG GAACCTTAGA |
| | AAGTTGTAAA |
| 1051 | GAACTCGTAA ATAACAAAAA ATCCGGGTTA CTGACCATTT |
| | ACTACCCTGA |
| 1101 | AGGACAGATC ATGGCGACCG AAGAGTATGA TAATGATCTT |
| | CTAATTAAAG |
| 1151 | GAGAGTACTT CCGCCCTGGA GACCGTCATC CCTACTCTAA |
| | AATAGATCGT |
| 1201 | GGTTGTGGGA CTGCAGTATT TTTCTCGTCG GCGGGAACTA |
| | TTACTAAAAA |
| 1251 | AATCCCCTAT CAGGACGGCA AACCTTTGCT CAACTAG |

The PSORT algorithm predicts a periplasmic location (0.688).

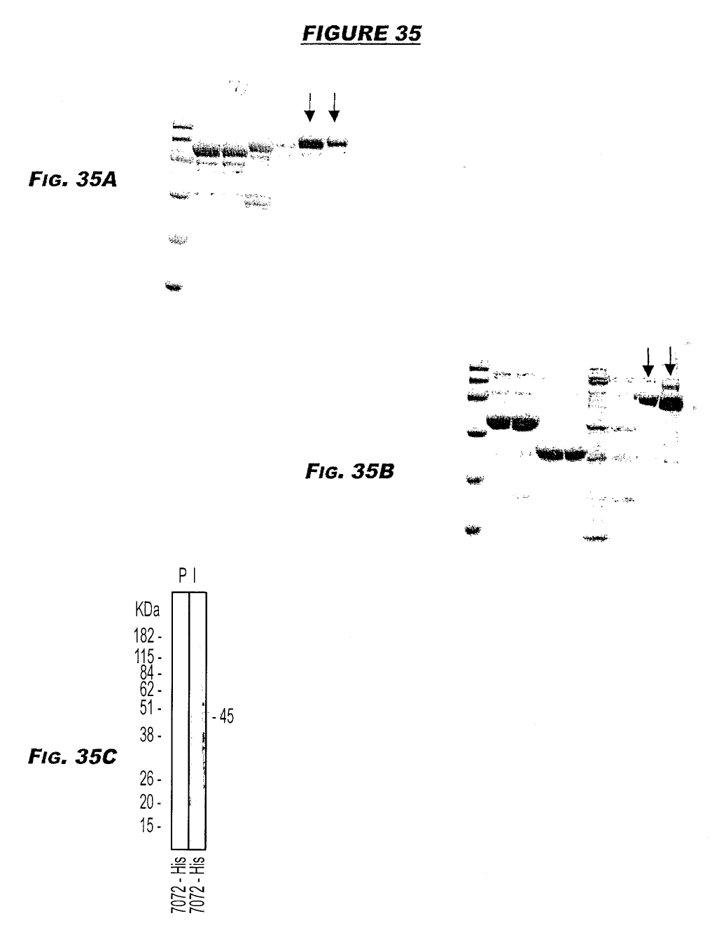

The protein was expressed in E. coli and purified as a his-tag product (FIG. 35A) and as a GST-fusion product (FIG. 35B). The recombinant his-tag protein was used to immunise mice, whose sera were used in a Western blot (FIG. 35C) and for FACS analysis.

These experiments show that cp7072 is a useful immunogen. These properties are not evident from the sequence alone.

### Example 36

The following C. pneumoniae protein (PID 4376879) was expressed <SEQ ID 71; cp6879>:

| | |
|---|---|
| 1 | MATPAQKSPT FQDPSFVREL GSNHPVFSPL TLEERGEMAI |
| | ARVQQCGWNH |
| 51 | TIVKVSLIIL ALLTILGGGL LVGLLPAVPM FIGTGLIALG |
| | AVIFALALIL |
| 101 | CLYDSQGLPE ELPPVPEPQQ IQIEDLRNET REVLEGTLLE |
| | VLLKDRDAKD |
| 151 | PAVPQVVVDC EKRLGMLDRK LRREEEILYR STAELKDEER |
| | YEFLLELLEM |
| 201 | RSLVADRLEF NRRSYERFVQ GIMTVRSEEG EKEISRLQDL |
| | ISLQQQTVQD |
| 251 | LRSRIDDEQK RCWTALQRIN QSQKDIQRAH DREASQRACE |
| | GTEMDCAERQ |
| 301 | QLEKDLRRQL KSMQEWIEMR GTIHQQEKAW RKQNAKLERL |
| | QEDLRLTGIA |
| 351 | FDEQSLFYRE YKEKYLSQKL DMQKILQEVN AEKSEKACLE |
| | SLVHDYEKQL |
| 401 | EQKDANLKKA AAVWEEELGK QQQEDYEQTQ EIRRLSTFIL |
| | EYQDSLREAE |
| 451 | KVEKDFQELQ QRYSRLQEEK QVKEKILEES MNHFADLFEK |
| | AQKENMAYKK |
| 501 | KLADLEGAAA PTEIGEDDDW VLTDSASLSQ KKIRELVEEN |
| | QELLKALAFK |
| 551 | SNELTQLVAD AVEAEKEISK LREHIEEQKE GLRALDKMHA |
| | QAIKDCEAAQ |
| 601 | RKCCDLESLL SPVREDAGMR FELEVELQRL QEENAQLRAE |
| | VERLEQEQFQ |
| 651 | G* |

The cp6879 nucleotide sequence <SEQ ID 72> is:

| | |
|---|---|
| 1 | ATGGCAACAC CCGCTCAAAA ATCCCCTACA TTTCAAGATC |
| | CTAGTTTTGT |
| 51 | AAGAGAGCTA GGCAGTAACC ACCCTGTCTT TTCCCCGCTA |
| | ACGCTTGAGG |
| 101 | AAAGAGGGGA GATGGCAATA GCTCGAGTCC AGCAGTGTGG |
| | ATGGAATCAT |
| 151 | ACAATTGTTA AGGTAAGTCT TATTATTCTT GCTCTTCTTA |
| | CTATTTTAGG |
| 201 | GGGAGGATTA CTCGTAGGAT TGCTGCCAGC AGTTCCTATG |
| | TTTATTGGAA |
| 251 | CAGGTCTGAT TGCTTTGGGA GCCGTTATAT TTGCTTTGGC |
| | TTTGATTTTA |
| 301 | TGTCTTTATG ATTCTCAGGG CCTTCCTGAG GAACTCCCTC |
| | CGGTTCCTGA |
| 351 | ACCACAACAA ATTCAGATTG AAGATTTAAG AAACGAGACC |
| | AGAGAAGTTC |
| 401 | TTGAAGGGAC TCTTTTAGAG GTTCTCTTAA AGGATAGAGA |
| | CGCTAAGGAC |
| 451 | CCTGCGGTGC CCCAGGTGGT TGTAGACTGT GAAAAGCGTC |
| | TTGGAATGTT |
| 501 | GGATCGTAAG CTGCGACGTG AAGAGGAGAT TCTGTATCGC |
| | TCGACGGCCC |
| 551 | ATCTTAAAGA CGAGGAAAGG TATGAGTTCT TGCTGGAGCT |
| | CTTGGAAATG |
| 601 | CGTAGTCTGG TTGCCGATCG GCTAGAATTT AACCGTAGAA |
| | GTTATGAGCG |
| 651 | ATTTGTTCAA GGAATTATGA CAGTTAGATC AGAGGAGGGG |
| | GAAAAAGAGA |
| 701 | TTTCTCGTCT ACAAGATCTA ATCAGTTTGC AGCAGCAGAC |
| | GGTGCAAGAT |
| 751 | TTAAGGAGTC GGATCGATGA CGAGCAGAAG AGATGCTGGA |
| | CGGCTTTACA |
| 801 | ACGTATTAAC CAATCTCAGA AGGATATACA ACGGGCTCAT |
| | GATCGCGAGG |
| 851 | CTTCGCAGCG TGCCTGTGAG GGCACAGAGA TGGATTGTGC |
| | AGAACGCCAG |
| 901 | CAACTGGAGA AGGATTTAAG GAGACAGCTG AAATCTATGC |
| | AGGAGTGGAT |
| 951 | TGAGATGAGG GGCACAATCC ATCAACAAGA GAAGGCTTGG |
| | CGTAAGCAGA |
| 1001 | ATGCCAAATT AGAAAGATTA CAAGAGGATC TGAGACTTAC |
| | TGGGATTGCT |
| 1051 | TTTGACGAAC AATCTCTGTT CTATCGCGAA TATAAAGAGA |
| | AATATCTGAG |
| 1101 | TCAGAAACTA GATATGCAAA AGATTTTACA GGAAGTCAAC |
| | GCAGAGAAAA |
| 1151 | GTGAGAAGGC TTGCTTAGAG AGTCTGGTCC ATGACTATGA |
| | GAAGCAGCTC |
| 1201 | GAACAAAAAG ATGCTAATCT GAAGAAAGCA GCAGCTGTTT |
| | GGGAAGAAGA |
| 1251 | ATTAGGGAAG CAGCAACAGG AAGACTACGA ACAAACCCAA |
| | GAAATTAGAC |
| 1301 | GTCTGAGTAC ATTCATTCTT GAGTACCAGG ACAGTCTGCG |
| | TGAGGCAGAA |
| 1351 | AAAGTTGAGA AAGATTTCCA AGAGCTACAA CAAAGGTATA |
| | GCCGTCTTCA |
| 1401 | AGAGGAGAAA CAGGTAAAAG AAAAAATCTT AGAAGAAAGT |
| | ATGAATCATT |
| 1451 | TTGCCGATCT CTTTGAGAAG GCTCAAAAGG AAAACATGGC |
| | CTACAAGAAG |
| 1501 | AAGTTAGCGG ATTTAGAGGG TGCCGCTGCT CCTACTGAGA |
| | TCGGTGAGGA |
| 1551 | CGATGACTGG GTACTCACAG ATTCTGCTTC TCTCAGCCAG |
| | AAGAAGATCC |
| 1601 | GCGAACTCGT GGAAGAGAAT CAAGAACTCC TGAAAGCACT |
| | TGCATTTAAA |
| 1651 | TCTAACGAAT TGACTCAACT GGTTGCCGAT GCTGTAGAAG |
| | CTGAAAAAGA |
| 1701 | AATCAGCAAG CTTCGAGAAC ACATAGAAGA GCAGAAAGAA |
| | GGATTACGAG |
| 1751 | CTCTTGATAA GATGCATGCA CAAGCGATCA AAGATTGCGA |
| | AGCTGCTCAG |
| 1801 | AGAAAATGCT GTGACCTTGA GAGCCTTCTC TCTCCTGTTC |
| | GAGAAGATGC |
| 1851 | TGGAATGAGA TTTGAGCTAG AGGTCGAGCT TCAAAGATTG |
| | CAAGAAGAAA |
| 1901 | ATGCACAGCT TAGAGCGGAG GTTGAAAGAC TAGAGCAAGA |
| | GCAATTTCAA |
| 1951 | GGATAA |

The PSORT algorithm predicts an inner membrane location (0.646).

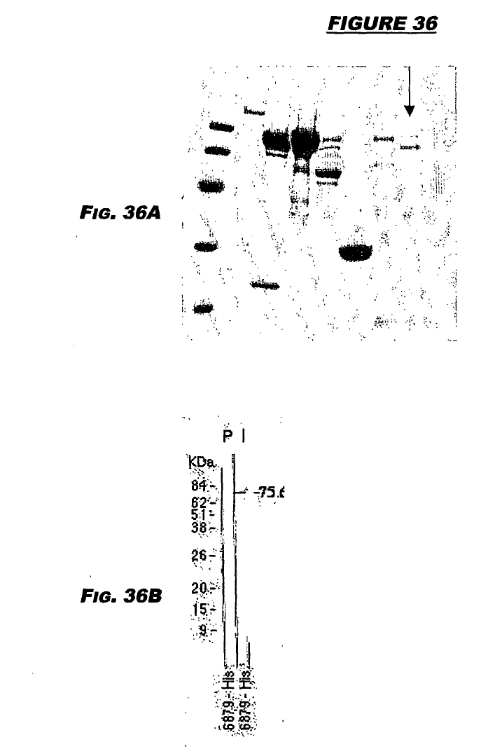

The protein was expressed in E. coli and purified as a his-tag product and as a GST-fusion product. The purified GST-fusion product is shown in FIG. 36A. The recombinant GST-fusion protein was used to immunise mice, whose sera were used in a Western blot (FIG. 36B) and for FACS analysis.

These experiments show that cp6879 is useful immunogen. These properties are not evident from the sequence alone.

### Example 37

The following C. pneumoniae protein (PID 4376767) was expressed <SEQ ID 73; cp6767>:

| | |
|---|---|
| 1 | MIKQIGRFFR AFIFIMPLSL TSCESKIDRN RIWIVGTNAT |
| | YPPFEYVDAQ |
| 51 | GEVVGFDIDL AKAISEKLGK QLEVREFAFD ALILNLKKHR |
| | IDAILAGMSI |
| 101 | TPSRQKEIAL LPYYGDEVQE LMVVSKRSLE TPVLPLTQYS |
| | SVAVQTGTFQ |
| 151 | EHYLLSQPGI CVRSFDSTLE VIMEVRYGKS PVAVLEPSVG |
| | RVVLKDFPNL |
| 201 | VATRLELPPE CWVLGCGLGV AKDRPEEIQT IQQAITDLKS |
| | EGVIQSLTKK |
| 251 | WQLSEVAYE* |

The cp6767 nucleotide sequence <SEQ ID 74> is:

| | |
|---|---|
| 1 | ATGATAAAAC AAATAGGCCG TTTTTTTAGA GCATTTATTT |
| | TTATAATGCC |
| 51 | TTTATCTTTA ACAAGTTGTG AGTCTAAAAT CGATCGAAAT |
| | CGCATCTGGA |
| 101 | TTGTAGGTAC GAATGCTACA TATCCTCCTT TTGAGTATGT |
| | GGATGCTCAG |
| 151 | GGGGAAGTTG TAGGTTTCGA TATAGATTTG GCAAAGGCAA |
| | TTAGTGAAAA |
| 201 | ACTTGGCAAG CAATTGGAAG TTAGAGAATT CGCTTTCGAT |
| | GCTTTAATTT |
| 251 | TAAATTTAAA AAAACATCGT ATCGATGCAA TTTTAGCAGG |
| | AATGTCCATT |
| 301 | ACTCCTTCGC GTCAGAAGGA AATCGCCCTG CTTCCCTATT |
| | ATGGCGATGA |
| 351 | GGTTCAAGAG CTGATGGTGG TTTCTAAGCG GTCTTTAGAG |
| | ACCCCTGTGC |
| 401 | TTCCCCTAAC ACAGTATTCT TCTGTTGCTG TTCAGACAGG |
| | AACGTTTCAG |
| 451 | GAGCATTATC TTTTATCTCA GCCCGGAATT TGTGTCCGTT |
| | CTTTTGATAG |
| 501 | CACCTTGGAG GTGATTATGG AAGTTCGTTA TGGGAAATCT |
| | CCGGTTGCCG |
| 551 | TTCTAGAACC CTCGGTAGGA CGTGTCGTTC TTAAAGACTT |
| | CCCTAATCTT |
| 601 | GTTGCAACAA GATTAGAGCT CCCTCCTGAA TGTTGGGTGT |
| | TGGGCTGTGG |
| 651 | TCTCGGCGTA GCTAAAGATC GTCCTGAAGA AATACAAACG |
| | ATTCAACAAG |
| 701 | CGATTACAGA TTTAAAGAGC GAAGGGGTGA TTCAATCTTT |
| | AACCAAGAAA |
| 751 | TGGCAACTTT CTGAAGTTGC TTACGAATAG |

The PSORT algorithm predicts an inner membrane location (0.083).

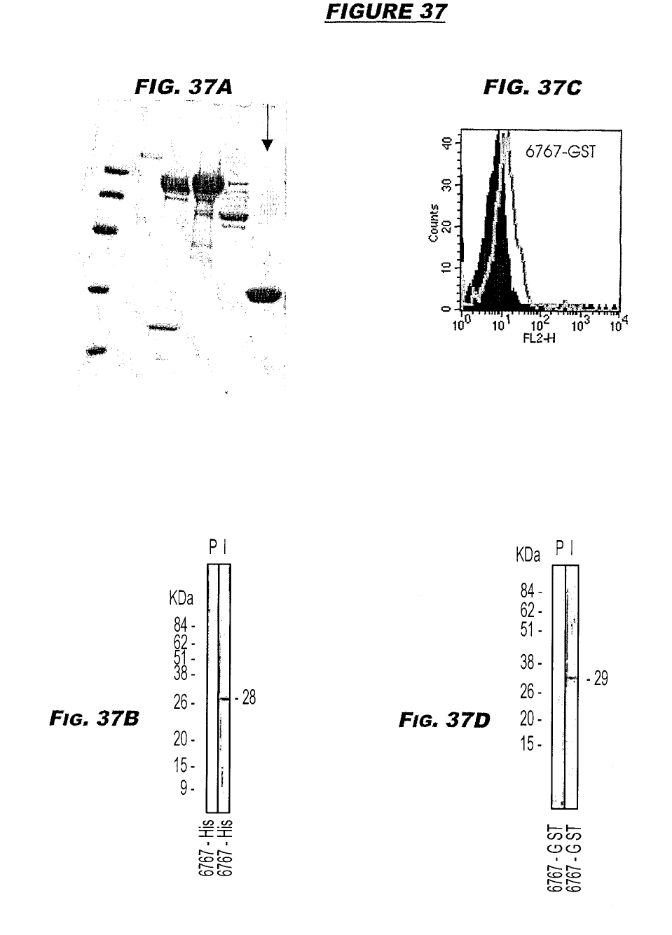

The protein was expressed in E. coli and purified as a his-tag product and as a GST-fusion product. The purified his-tag product is shown in FIG. 37A. The recombinant his-tag protein was used to immunise mice, whose sera were used in a Western blot (FIG. 37B) and for FACS analysis (FIG. 37C). The GST-fusion was also used in a Western blot (FIG. 37D).

The cp6767 protein was also identified in the 2D-PAGE experiment and showed good cross-reactivity with human sera, including sera from patients with pneumonitis.

These experiments show that cp6767 is a useful immunogen. These properties are not evident from the sequence alone.

### Example 38

The following C. pneumoniae protein (PID 4376717) was expressed <SEQ ID 75; cp6717>:

| | |
|---|---|
| 1 | MMSRLRFRLA ALGIFFILLV PNSVSAKTIV ASDKEKVGVL |
| | VYDNSVEAFQ |
| 51 | QILDCIDHAN FYVELCPCMT GGRTLKEMVD HLEARMDLVP |
| | ELCSYIIIQP |
| 101 | TFTDAEDQKL LKALKERHPN RFFYVFTGCP PSTSILAPNV |
| | IEMHIKLSII |
| 151 | DGKYCILGGT NFEEFMCTPG DEVPEKVDNP RLFVSGVRRP |
| | LAFRDQDIML |
| 201 | RSTAFGLQLR EEYHKQFAMW DYYAHHMWFI DNPEQFAGAC |
| | PPLTLEQAEE |
| 251 | TVFPGFDKHE DLVLVDSSKI RIVLGGPHDK QPNPVTQEYL |
| | KLIQGARSSV |
| 301 | KLAHMYFIPK DELLNALVDV SHNHGVHLSL ITNGCHELSP |
| | AITGPYAWGN |
| 351 | RINYFALLYG KRYPIWKKWF CEKLKPYERV SIYEFAIWET |
| | QLHKKCMIID |
| 401 | DEIFVIGSYN FGKKSDAFDY ESIVVIESPE VAAKANKVFN |
| | KDIGLSIPVS |
| 451 | HGDIFSWYFH SVHHTLGHLQ LTYMPA* |

A predicted signal peptide is highlighted.

The cp6717 nucleotide sequence <SEQ ID 76> is:

| | |
|---|---|
| 1 | ATGATGAGTC GGTTGCGTTT TCGCTTGGCA GCTCTTGGAA |
| | TATTTTTTAT |
| 51 | TTTGCTGGTT CCTAATTCTG TTTCAGCAAA GACAATCGTA |
| | GCTTCAGACA |
| 101 | AGGAGAAGGT TGGAGTTCTT GTTTATGACA ATAGTGTAGA |
| | GGCCTTTCAA |
| 151 | CAGATATTGG ATTGCATAGA TCATGCAAAT TTTTATGTAG |
| | AACTGTGTCC |
| 201 | CTGCATGACA GGAGGCCGAA CGCTTAAAGA GATGGTAGAT |
| | CACCTCGAGG |
| 251 | CTCGTATGGA TCTGGTTCCA GAGCTCTGTA GCTATATCAT |
| | TATCCAACCC |
| 301 | ACGTTTACCG ATGCTGAAGA CCAAAAATTA CTCAAAGCTC |
| | TCAAAGAACG |
| 351 | TCATCCCAAC CGGTTTTTCT ACGTTTTTAC AGGGTGCCCA |
| | CCCTCAACAA |
| 401 | GCATCCTCGC TCCTAATGTC ATTGAAATGC ATATCAAACT |
| | TTCTATCATC |
| 451 | GATGGGAAAT ATTGTATTTT AGGTGGTACC AATTTTGAAG |
| | AGTTTATGTG |
| 501 | CACTCCAGGG GATGAGGTTC CTGAGAAAGT GGATAACCCA |
| | CGTTTATTTG |
| 551 | TCAGTGGAGT GCGTCGGCCC CTAGCATTTC GTGATCAGGA |
| | TATCATGTTG |
| 601 | CGTTCTACAG CATTCGGTTT GCAGCTCAGA GAAGAATATC |
| | ATAAGCAATT |
| 651 | TGCTATGTGG GACTACTATG CACATCATAT GTGGTTCATT |
| | GATAATCCTG |
| 701 | AACAGTTTGC AGGCGCCTGT CCTCCACTGA CTTTAGAACA |
| | AGCCGAGGAG |
| 751 | ACAGTATTTC CTGGATTTGA CAAACATGAA GATCTTGTTC |
| | TTGTCGACTC |
| 801 | TTCCAAGATC AGGATAGTTT TAGGTGGTCC CCACGATAAG |
| | CAACCCAATC |
| 851 | CTGTGACTCA AGAATATTTG AAACTTATCC AGGGAGCTAG |
| | ATCTTCTGTG |
| 901 | AAGCTTGCTC ACATGTATTT CATCCCTAAG GACGAGCTTT |
| | TAAATGCTCT |
| 951 | TGTCGACGTT TCTCATAATC ACGGTGTTCA TCTGAGTTTA |
| | ATTACGAACG |
| 1001 | GCTGTCATGA ATTAAGTCCT GCAATTACAG GACCCTATGC |
| | TTGGGGAAAC |
| 1051 | CGTATTAACT ATTTCGCCTT GCTCTATGGG AAACGGTATC |
| | CTCTTTGGAA |
| 1101 | AAAATGGTTT TGCGAAAAGC TAAAACCTTA TGAGCGGGTT |
| | TCTATTTATG |
| 1151 | AGTTTGCTAT TTGGGAAACG CAGTTGCACA AGAAGTGTAT |
| | GATTATCGAT |
| 1201 | GATGAAATTT TTGTGATCGG AAGTTATAAT TTTGGAAAGA |
| | AAAGTGATGC |
| 1251 | CTTTGATTAC GAAAGTATTG TAGTTATCGA ATCTCCAGAA |
| | GTCGCTGCAA |
| 1301 | AAGCTAACAA AGTCTTCAAT AAAGATATCG GATTGTCGAT |
| | TCCTGTAAGT |
| 1351 | CATGGCGACA TTTTCTCTTG GTATTTCCAT TCCGTACACC |
| | ACACTTTGGG |
| 1401 | ACATTTGCAG CTGACCTATA TGCCAGCCTA G |

The PSORT algorithm predicts a periplasmic location (0.939).

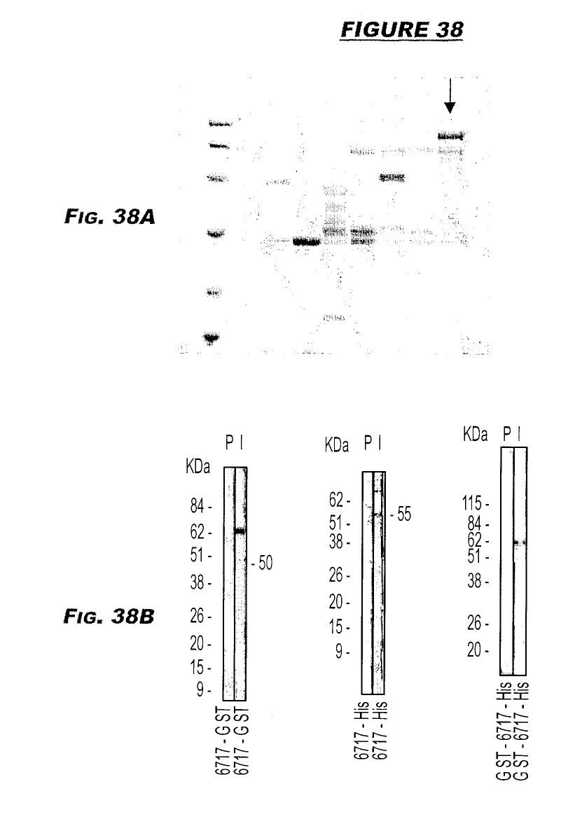

The protein was expressed in E. coli and purified as a GST-fusion (FIG. 38A), as a his-tagged protein, and as a GST/his fusion product. The proteins were used to immunise mice, whose sera were used in a Western blot (FIG. 38B) and for FACS analysis.

These experiments show that cp6717 is a useful immunogen. These properties are not evident from the sequence alone.

### Example 39

The following C. pneumoniae protein (PID 4376577) was expressed <SEQ ID 77; cp6577>:

| | |
|---|---|
| 1 | MKKLLFSTFL LVLGSTSAAH ANLGYVNLKR CLEESDLGKK |
| | ETEELEAMKQ |
| 51 | QFVKNAEKIE EELTSIYNKL QDEDYMESLS DSASEELRKK |
| | FEDLSGEYNA |
| 101 | YQSQYYQSIN QSNVKRIQKL IQEVKIAAES VRSKEKLEAI |
| | LNEEAVLAIA |
| 151 | PGTDKTTEII AILNESFKKQ N* |

A predicted signal peptide is highlighted.

The cp6577 nucleotide sequence <SEQ ID 78> is:

| | |
|---|---|
| 1 | ATGAAAAAAT TATTATTTTC TACATTTCTT CTTGTTTTAG |
| | GATCAACAAG |
| 51 | CGCAGCTCAT GCAAATTTAG GCTATGTTAA TTTAAAGCGA |
| | TGTCTTGAAG |
| 101 | AATCCGATCT AGGTAAAAAG GAAACTGAAG AATTGGAAGC |
| | TATGAAACAG |
| 151 | CAGTTTGTAA AAAATGCTGA GAAAATAGAA GAAGAACTCA |
| | CTTCTATTTA |
| 201 | TAATAAGTTG CAAGATGAAG ATTACATGGA AAGCCTATCG |
| | GATTCTGCCT |
| 251 | CTGAAGAGTT GCGAAAGAAA TTCGAAGATC TTTCAGGAGA |
| | GTACAATGCG |
| 301 | TACCAGTCTC AGTACTATCA ATCTATCAAT CAAAGTAATG |
| | TAAAACGCAT |
| 351 | TCAAAAACTC ATCCAAGAAG TAAAAATAGC TGCAGAATCA |
| | GTGCGGTCCA |
| 401 | AAGAAAAACT AGAAGCTATC CTTAATGAAG AAGCTGTCTT |
| | AGCAATAGCA |
| 451 | CCTGGGACTG ATAAAACAAC CGAAATTATT GCTATTCTTA |
| | ACGAATCTTT |
| 501 | CAAAAAACAA AACTAG |

The PSORT algorithm predicts a periplasmic space location (0.932).

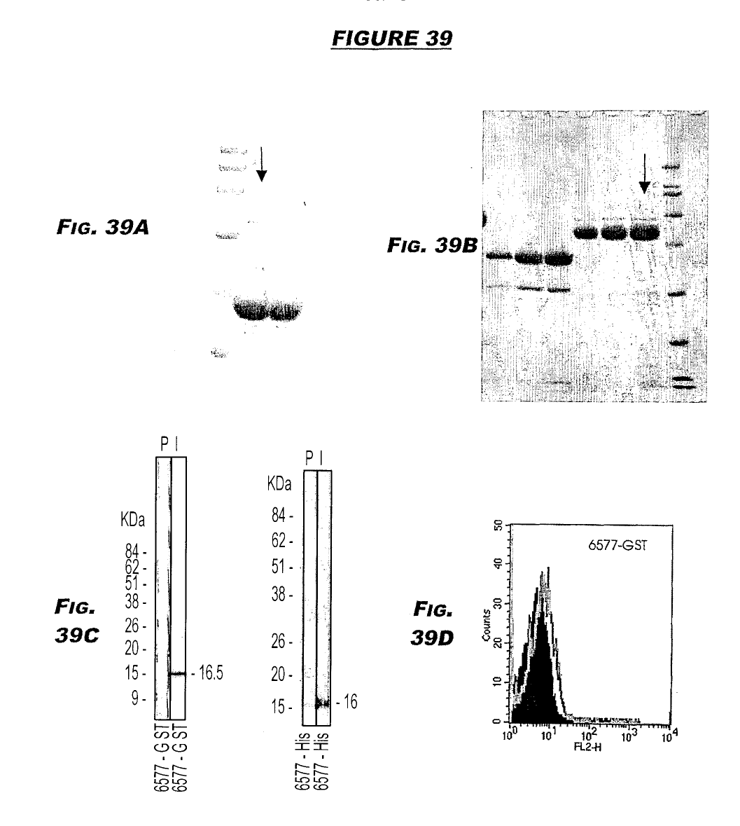

The protein was expressed in E. coli and purified as a his-tag product (FIG. 39A) and as a GST-fusion product (FIG. 39B). The recombinant GST-fusion protein was used to immunise mice, whose sera were used in a Western blot (FIG. 39C) and for FACS analysis.

The cp6577 protein was also identified in the 2D-PAGE experiment.

These experiments show that cp6577 is a useful immunogen. These properties are not evident from the sequence alone.

### Example 40

The following C. pneumoniae protein (PID 4376446) was expressed <SEQ ID 79; cp6446>:

| | |
|---|---|
| 1 | MKQPMSLIFS SVCLGLGLGS LSSCNQKPSW NYHNTSTSEE |
| | FFVHGNKSVS |
| 51 | QLPHYPSAFR TTQIFSEEHN DPYVVAKTDE ESRKIWREIH |
| | KNLKIKGSYI |
| 101 | PISTYGSLMH PKSAALTLKT YRPHPIWING YERSFNIDTG |
| | KYLKNGSRRR |
| 151 | TSHDGPKNRA VLNLIKSSGR RCNAIGLEMT EEDFVIARRR |
| | EGVYSLYPVE |
| 201 | VCSYPQGNPF VIAYAWIADE SACSKEVLPV KGYYSLVWES |
| | VSSSDSLNAF |
| 251 | GDSFAEDYLR STFLANGTSI LCVHESYKKV PPQP* |

A predicted signal peptide is highlighted.

The cp6446 nucleotide sequence <SEQ ID 80> is:

| | |
|---|---|
| 1 | ATGAAACAGC CCATGTCTCT TATCTTTTCA AGTGTATGTT |
| | TAGGATTAGG |
| 51 | TCTTGGATCT CTTTCCTCCT GTAATCAAAA GCCCTCTTGG |
| | AATTATCACA |
| 101 | ACACTTCAAC GAGCGAAGAA TTCTTTGTTC ATGGAAATAA |
| | GAGTGTTTCG |
| 151 | CAACTGCCTC ATTATCCTTC TGCATTTCGT ACGACTCAAA |
| | TCTTTTCTGA |
| 201 | AGAGCACAAT GATCCTTATG TCGTAGCTAA GACTGATGAA |
| | GAGTCTCGTA |
| 251 | AAATTTGGAG AGAAATCCAT AAAAATCTCA AAATCAAAGG |
| | TTCTTACATT |
| 301 | CCCATATCGA CTTATGGAAG TCTGATGCAC CCAAAATCAG |
| | CAGCTCTTAC |
| 351 | ATTAAAAACG TATCGTCCAC ATCCTATTTG GATAAATGGA |
| | TACGAGCGTT |
| 401 | CTTTTAATAT AGACACAGGA AAGTACTTAA AAAACGGAAG |
| | TCGCCGTAGA |
| 451 | ACTTCTCACG ATGGTCCGAA AAATCGAGCT GTACTGAATC |
| | TCATTAAATC |
| 501 | TTCGGGACGA CGCTGTAATG CTATAGGCCT TGAGATGACA |
| | GAAGAAGACT |
| 551 | TTGTAATAGC TAGAAGGCGA GAAGGTGTTT ATAGCCTGTA |
| | TCCCGTTGAA |
| 601 | GTGTGCTCGT ATCCTCAGGG GAATCCTTTT GTCATTGCTT |
| | ATGCCTGGAT |
| 651 | TGCAGATGAG AGTGCTTGCT CAAAAGAGGT CCTACCTGTA |
| | AAAGGGTACT |
| 701 | ATTCTTTAGT CTGGGAAAGC GTTTCTTCCT CTGATTCTCT |
| | GAATGCTTTT |
| 751 | GGAGATTCCT TTGCAGAGGA CTACCTCAGA AGCACGTTTT |
| | TAGCAAACGG |
| 801 | AACTTCTATA CTCTGTGTTC ATGAAAGCTA TAAGAAAGTT |
| | CCTCCTCAGC |
| 851 | CCTAA |

The PSORT algorithm predicts an inner membrane location (0.177).

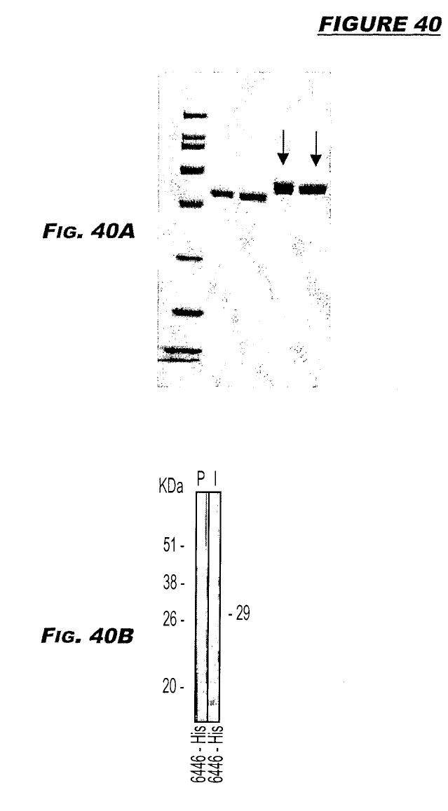

The protein was expressed in E. coli and purified as a his-tag product and a GST-fusion product. The GST-fusion product is shown in FIG. 40A. The recombinant his-tag protein was used to immunise mice, whose sera were used in a Western blot (FIG. 40B) and for FACS analysis.

These experiments show that cp6446 is a useful immunogen. These properties are not evident from the sequence alone.

### Example 41

The following C. pneumoniae protein (PID 4377108) was expressed <SEQ ID 81; cp7108>:

| | |
|---|---|
| 1 | MSKKIKVLGH LTLCTLFRGV LCAAALSNIG YASTSQESPY |
| | QKSIEDWKGY |
| 51 | TFTDLELLSK EGWSEAHAVS GNGSRIVGAS GAGQGSVTAV |
| | IWESHLIKHL |
| 101 | GTLGGEASSA EGISKDGEVV VGWSDTREGY THAFVFDGRD |
| | MKDLGTLGAT |
| 151 | YSVARGVSGD GSIIVGVSAT ARGEDYGWQV GVKWEKGKIK |
| | QLKLLPQGLW |
| 201 | SEANAISEDG TVIVGRGEIS RNHIVAVKWN KNAVYSLGTL |
| | GGSVASAEAI |
| 251 | SANGKVIVGW STTNNGETHA FMHKDETMHD LGTLGGGFSV |
| | ATGVSADGRA |
| 301 | IVGFSAVKTG EIHAFYYAEG EMEDLTTLGG EEARVFDISS |
| | EGNDIIGSIK |
| 351 | TDAGAERAYL FHIHK* |

A predicted signal peptide is highlighted.

The cp7108 nucleotide sequence <SEQ ID 82> is:

| | |
|---|---|
| 1 | ATGAGTAAGA AGATAAAGGT TCTAGGTCAT TTGACGCTCT |
| | GCACTCTGTT |
| 51 | TAGAGGAGTG CTGTGTGCAG CGGCCCTTTC CAACATAGGA |
| | TATGCGAGTA |
| 101 | CTTCTCAGGA ATCACCATAT CAGAAGTCTA TAGAAGACTG |
| | GAAAGGGTAT |
| 151 | ACCTTTACAG ATCTTGAGTT ACTGAGTAAG GAAGGGTGGT |
| | CTGAAGCTCA |
| 201 | TGCAGTTTCT GGAAATGGCA GTAGAATTGT AGGAGCTTCG |
| | GGAGCTGGCC |
| 251 | AAGGTAGTGT GACTGCTGTC ATATGGGAAA GTCACCTGAT |
| | AAAACATCTC |
| 301 | GGCACTTTAG GTGGCGAGGC TTCATCTGCA GAGGGAATTT |
| | CAAAGGATGG |
| 351 | AGAGGTGGTC GTTGGGTGGT CAGATACTAG AGAGGGATAT |
| | ACTCATGCCT |
| 401 | TTGTCTTCGA CGGTAGAGAT ATGAAAGATC TCGGTACTCT |
| | AGGAGCTACC |
| 451 | TATTCTGTAG CAAGGGGTGT TTCTGGAGAT GGTAGTATCA |
| | TCGTAGGAGT |
| 501 | CTCTGCAACT GCTCGTGGAG AGGATTACGG ATGGCAAGTT |
| | GGTGTCAAGT |
| 551 | GGGAAAAAGG GAAAATCAAA CAATTGAAGT TGTTGCCTCA |
| | AGGTCTCTGG |
| 601 | TCTGAGGCGA ATGCAATCTC TGAGGATGGT ACGGTGATTG |
| | TCGGGAGAGG |
| 651 | GGAAATCTCT CGCAATCACA TCGTTGCTGT AAAATGGAAT |
| | AAAAATGCTG |
| 701 | TGTATAGTTT GGGGACTCTC GGAGGTAGTG TCGCTTCAGC |
| | AGAGGCTATA |
| 751 | TCGGCAAATG GGAAAGTAAT TGTAGGATGG TCCACGACTA |
| | ATAATGGTGA |
| 801 | GACTCATGCC TTTATGCACA AAGATGAGAC AATGCACGAT |
| | CTCGGCACTC |
| 851 | TAGGAGGAGG TTTTTCTGTC GCAACTGGAG TTTCTGCTGA |
| | TGGGAGAGCC |
| 901 | ATCGTAGGAT TTTCAGCAGT GAAGACCGGA GAAATTCATG |
| | CTTTTTACTA |
| 951 | TGCAGAAGGA GAAATGGAGG ATTTAACAAC TTTGGGAGGG |
| | GAAGAAGCTC |
| 1001 | GAGTGTTCGA CATATCTAGC GAAGGAAACG ATATCATTGG |
| | CTCTATAAAA |
| 1051 | ACTGACGCTG GAGCTGAACG CGCCTATCTG TTCCATATAC |
| | ATAAATAA |

The PSORT algorithm predicts an outer membrane location (0.921).

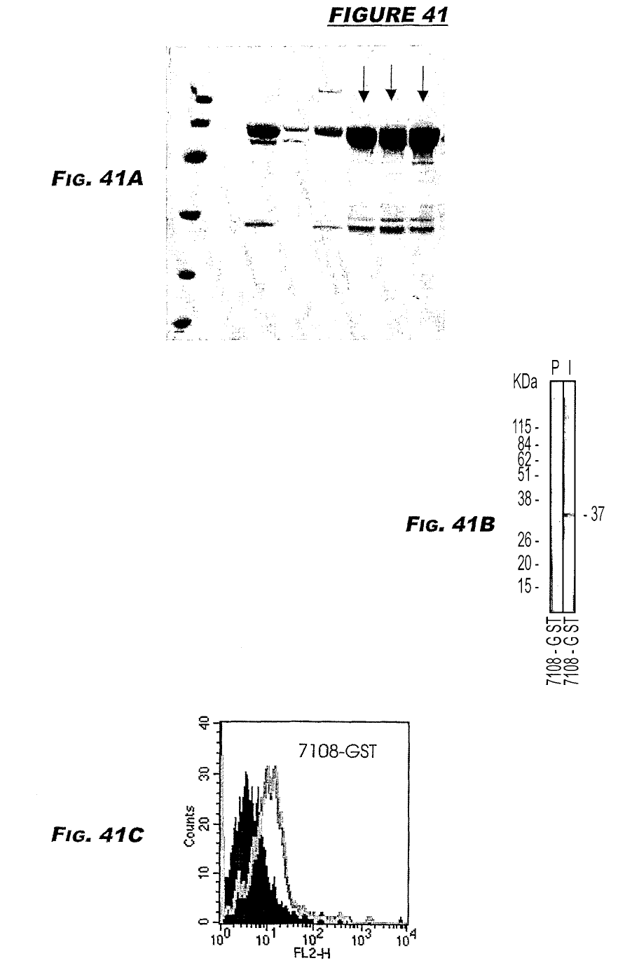

The protein was expressed in E. coli and purified as a GST-fusion product, as shown in FIG. 41A. The recombinant protein was used to immunise mice, whose sera were used in a Western blot (FIG. 41B) and for FACS analysis (FIG. 41C). A his-tagged protein was also expressed.

The cp7108 protein was also identified in the 2D-PAGE experiment.

These experiments show that cp7108 is a surface-exposed and immunoaccessible protein, and that it is a useful immunogen. These properties are not evident from the sequence alone.

### Example 42

The following C. pneumoniae protein (PID 4377287) was expressed <SEQ ID 83; cp7287>:

| | |
|---|---|
| 1 | MVAKKTVRSY RSSFSHSVIV AILSAGAIFE AHSLHSSELD |
| | LGVFNKQFEE |
| 51 | HSAHVEEAQT SVLKGSDPVN PSQKESEKVL YTQVPLTQGS |
| | SGESLDLADA |
| 101 | NFLEHFQHLF EETTVFGIDQ KLVWSDLDTR NFSQPTQEPD |
| | TSNAVSEKIS |
| 151 | SDTKENRKDL ETEDPSKKSG LDEVSSDLPK SPETAVAAIS |
| | EDLEISENIS |
| 201 | ARDPLQGLAF FYKNTSSQSI SEKDSSFQGI IFSGSGANSG |
| | LGFENLKAPK |
| 251 | SGAAVYSDRD IVFENLVKGL SFISCESLED GSAAGVNIVV |
| | THCGDVTLTD |
| 301 | CATGLDLEAL RLVKDFSRGG AVFTARNHEV QNNLAGGILS |
| | VVGNKGAIVV |
| 351 | EKNSAEKSNG GAFACGSFVY SNNENTALWK ENQALSGGAI |
| | SSASDIDIQG |
| 401 | NCSAIEFSGN QSLIALGEHI GLTDFVGGGA LAAQGTLTLR |
| | NNAVVQCVKN |
| 451 | TSKTHGGAIL AGTVDLNETI SEVAFKQNTA ALTGGALSAN |
| | DKVIIANNFG |
| 501 | EILFEQNEVR NHGGAIYCGC RSNPKLEQKD SGENINIIGN |
| | SGAITFLKNK |
| 551 | ASVLEVMTQA EDYAGGGALW GHNVLLDSNS GNIQFIGNIG |
| | GSTFWIGEYV |
| 601 | GGGAILSTDR VTISNNSGDV VFKGNKGQCL AQKYVAPQET |
| | APVESDASST |
| 651 | NKDEDSLNAC SHGDHYPPKT VEEEVPPSLL EEHPVVSSTD |
| | IRGGGAILAQ |
| 701 | HIFITDNTGN LRFSGNLGGG EESSTVGDLA IVGGGALLST |
| | NEVNVCSNQN |
| 751 | VVFSDNVTSN GCDSGGAILA KKVDISANHS VEFVSNGSGK |
| | FGGAVCALNE |
| 801 | SVNITDNGSA VSFSKNRTRL GGAGVAAPQG SVTICGNQGN |
| | IAFKENFVFG |
| 851 | SENQRSGGGA IIANSSVNIQ DNAGDILFVS NSTGSYGGAI |
| | FVGSLVASEG |
| 901 | SNPRTLTITG NSGDILFAKN STQTAASLSE KDSFGGGAIY |
| | TQNLKIVKNA |
| 951 | GNVSFYGNRA PSGAGVQIAD GSTVCLEAFG GDILFEGNIN |
| | FDGSFNAIHL |
| 1001 | CGNDSKIVEL SAVQDKNIIF QDAITYEENT IRGLPDKDVS |
| | PLSAPSLIFN |
| 1051 | SKPQDDSAQH HEGTIRFSRG VSKIPQIAAI QEGTLALSQN |
| | AELWLAGLKQ |
| 1101 | ETGSSIVLSA GLILRIFDSQ VDSSAPLPTE NKEETLVSAG |
| | VQINMSSPTP |
| 1151 | NKDKAVDTPV LADIISITVD LSSFVPEQDG TLPLPPEIII |
| | PKGTKLHSNA |
| 1201 | IDLKIIDPTN VGYENHALLS SHKDIPLISL KTAEGMTGTP |
| | TADASLSNIK |
| 1251 | IDVSLPSITP ATYGHTGVWS ESKMEDGRLV VGWQPTGYKL |
| | NPEKQGALVL |
| 1301 | NNLWSHYTDL RALKQEIFAH HTIAQRMELD FSTNVWGSGL |
| | GVVEDCQNIG |
| 1351 | EFDGFKHHLT GYALGLDTQL VEDFLIGGCF SQFFGKTESQ |
| | SYKAKNDVKS |
| 1401 | YMGAAYAGIL AGPWLIKGAF VYGNINNDLT TDYGTLGIST |
| | GSWIGKGFIA |
| 1451 | GTSIDYRYIV NPRRFISAIV STVVPFVEAE YVRIDLPEIS |
| | EQGKEVRTFQ |
| 1501 | KTRFENVAIP FGFAIEHAYS RGSRAEVNSV QLAYVFDVYR |
| | KGPVSLITLK |
| 1551 | DAAYSWKSYG VDIPCKAWKA RLSNNTEWNS YLSTYLAFNY |
| | EWREDLIAYD |
| 1601 | FNGGIRIIF* |

A predicted signal peptide is highlighted.

The cp7287 nucleotide sequence <SEQ ID 84> is:

| | |
|---|---|
| 1 | ATGGTAGCGA AAAAAACAGT ACGATCTTAT AGGTCTTCAT |
| | TTTCTCATTC |
| 51 | CGTAATAGTA GCAATATTGT CAGCAGGCAT TGCTTTTGAA |
| | GCACATTCCT |
| 101 | TACACAGCTC AGAACTAGAT TTAGGTGTAT TCAATAAACA |
| | GTTTGAGGAA |
| 151 | CATTCTGCTC ATGTTGAAGA GGCTCAAACA TCTGTTTTAA |
| | AGGGATCAGA |
| 201 | TCCTGTAAAT CCCTCTCAGA AAGAATCCGA GAAGGTTTTG |
| | TACACTCAAG |
| 251 | TGCCTCTTAC CCAAGGAAGC TCTGGAGAGA GTTTGGATCT |
| | CGCCGATGCT |
| 301 | AATTTCTTAG AGCATTTTCA GCATCTTTTT GAAGAGACTA |
| | CAGTATTTGG |
| 351 | TATCGATCAA AAGCTGGTTT GGTCAGATTT AGATACTAGG |
| | AATTTTTCCC |
| 401 | AACCCACTCA AGAACCTGAT ACAAGTAATG CTGTAAGTGA |
| | GAAAATCTCC |
| 451 | TCAGATACCA AAGAGAATAG AAAAGACCTA GAGACTGAAG |
| | ATCCTTCAAA |
| 501 | AAAAAGTGGC CTTAAAGAAG TTTCATCAGA TCTCCCTAAA |
| | AGTCCTGAAA |
| 551 | CTGCAGTAGC AGCTATTTCT GAAGATCTTG AAATCTCAGA |
| | AAACATTTCA |
| 601 | GCAAGAGATC CTCTTCAGGG TTTAGCATTT TTTTATAAAA |
| | ATACATCTTC |
| 651 | TCAGTCTATC TCTGAAAAGG ATTCTTCATT TCAAGGAATT |
| | ATCTTTTCTG |
| 701 | GTTCAGGAGC TAATTCAGGG CTAGGTTTTG AAAATCTTAA |
| | GGCGCCGAAA |
| 751 | TCTGGGGCTG CAGTTTATTC TGATCGAGAT ATTGTTTTTG |
| | AAAATCTTGT |
| 801 | TAAAGGATTG AGTTTTATAT CTTGTGAATC TTTAGAAGAT |
| | GGCTCTGCCG |
| 851 | CAGGTGTAAA CATTGTTGTG ACCCATTGTG GTGATGTAAC |
| | TCTCACTGAT |
| 901 | TGTGCCACTG GTTTAGACCT TGAAGCTTTA CGTCTGGTTA |
| | AAGATTTTTC |
| 951 | TCGTGGAGGA GCTGTTTTCA CTGCTCGCAA CCATGAAGTG |
| | CAAAATAACC |
| 1001 | TTGCAGGTGG AATTCTATCC GTTGTAGGCA ATAAAGGAGC |
| | TATTGTTGTA |
| 1051 | GAGAAAAATA GTGCTGAGAA GTCCAATGGA GGAGCTTTTG |
| | CTTGCGGAAG |
| 1101 | TTTTGTTTAC AGTAACAACG AAAACACCGC CTTGTGGAAA |
| | GAAAATCAAG |
| 1151 | CATTATCAGG AGGAGCCATA TCCTCAGCAA GTGATATTGA |
| | TATTCAAGGG |
| 1201 | AACTGTAGCG CTATTGAATT TTCAGGAAAC CAGTCTCTAA |
| | TTGCTCTTGG |
| 1251 | AGAGCATATA GGGCTTACAG ATTTTGTAGG TGGAGGAGCT |
| | TTAGCTGCTC |
| 1301 | AAGGGACGCT TACCTTAAGA AATAATGCAG TAGTGCAATG |
| | TGTTAAAAAC |
| 1351 | ACTTCTAAAA CACATGGTGG AGCTATTTTA GCAGGTACTG |
| | TTGATCTCAA |
| 1401 | CGAAACAATT AGCGAAGTTG CCTTTAAGCA GAATACAGCA |
| | GCTCTAACTG |
| 1451 | GAGGTGCTTT AAGTGCAAAT GATAAGGTTA TAATTGCAAA |
| | TAACTTTGGA |
| 1501 | GAAATTCTTT TTGAGCAAAA CGAAGTGAGG AATCACGGAG |
| | GAGCCATTTA |
| 1551 | TTGTGGATGT CGATCTAATC CTAAGTTAGA ACAAAAGGAT |
| | TCTGGAGAGA |
| 1601 | ACATCAATAT TATTGGAAAC TCCGGAGCTA TCACTTTTTT |
| | AAAAAATAAG |
| 1651 | GCTTCTGTTT TAGAAGTGAT GACACAAGCT GAAGATTATG |
| | CTGGTGGAGG |
| 1701 | CGCTTTATGG GGGCATAATG TTCTTCTAGA TTCCAATAGT |
| | GGGAATATTC |
| 1751 | AATTTATAGG AAATATAGGT GGAAGTACCT TCTGGATAGG |
| | AGAATATGTC |
| 1801 | GGTGGTGGTG CGATTCTCTC TACTGATAGA GTGACAATTT |
| | CTAATAACTC |
| 1851 | TGGAGATGTT GTTTTTAAAG GAAACAAAGG CCAATGTCTT |
| | GCTCAAAAAT |
| 1901 | ATGTAGCTCC TCAAGAAACA GCTCCCGTGG AATCAGATGC |
| | TTCATCTACA |
| 1951 | AATAAAGACG AGAAGAGCCT TAATGCTTGT AGTCATGGAG |
| | ATCATTATCC |
| 2001 | TCCTAAAACT GTAGAAGAGG AAGTGCCACC TTCATTGTTA |
| | GAAGAACATC |
| 2051 | CTGTTGTTTC TTCGACAGAT ATTCGTGGTG GTGGGGCCAT |
| | TCTAGCTCAA |
| 2101 | CATATCTTTA TTACAGATAA TACAGGAAAT CTGAGATTCT |
| | CTGGGAACCT |
| 2151 | TGGTGGTGGT GAAGAGTCTT CTACTGTCGG TGATTTAGCT |
| | ATCGTAGGAG |
| 2201 | GAGGTGCTTT GCTTTCTACT AATGAAGTTA ATGTTTGCAG |
| | TAACCAAAAT |
| 2251 | GTTGTTTTTT CTGATAACGT GACTTCAAAT GGTTGTGATT |
| | CAGGGGGAGC |
| 2301 | TATTTTAGCT AAAAAAGTAG ATATCTCCGC GAACCACTCG |
| | GTTGAATTTG |
| 2351 | TCTCTAATGG TTCAGGGAAA TTCGGTGGTG CCGTTTGCGC |
| | TTTAAACGAA |
| 2401 | TCAGTAAACA TTACGGACAA TGGCTCGGCA GTATCATTCT |
| | CTAAAAATAG |
| 2451 | AACACGTCTT GGCGGTGCTG GAGTTGCAGC TCCTCAAGGC |
| | TCTGTAACGA |
| 2501 | TTTGTGGAAA TCAGGGAAAC ATAGCATTTA AAGAGAACTT |
| | TGTTTTTGGC |
| 2551 | TCTGAAAATC AAAGATCAGG TGGAGGAGCT ATCATTGCTA |
| | ACTCTTCTGT |
| 2601 | AAATATTCAG GATAACGCAG GAGATATCCT ATTTGTAAGT |
| | AACTCTACGG |
| 2651 | GATCTTATGG AGGTGCTATT TTTGTAGGAT CTTTGGTTGC |
| | TTCTGAAGGC |
| 2701 | AGCAACCCAC GAACGCTTAC AATTACAGGC AACAGTGGGG |
| | ATATCCTATT |
| 2751 | TGCTAAAAAT AGCACGCAAA CAGCCGCTTC TTTATCAGAA |
| | AAAGATTCCT |
| 2801 | TTGGTGGAGG GGCCATCTAT ACACAAAACC TCAAAATTGT |
| | AAAGAATGCA |
| 2851 | GGGAACGTTT CTTTCTATGG CAACAGAGCT CCTAGTGGTG |
| | CTGGTGTCCA |
| 2901 | AATTGCAGAC GGAGGAACTG TTTGTTTAGA GGCTTTTGGA |
| | GGAGATATCT |
| 2951 | TATTTGAAGG GAATATCAAT TTTGATGGGA GTTTCAATGC |
| | GATTCACTTA |
| 3001 | TGCGGGAATG ACTCAAAAAT CGTAGAGCTT TCTGCTGTTC |
| | AAGATAAAAA |
| 3051 | TATTATTTTC CAAGATGCAA TTACTTATGA AGAGAACACA |
| | ATTCGTGGCT |
| 3101 | TGCCAGATAA AGATGTCAGT CCTTTAAGTG CCCCTTCATT |
| | AATTTTTAAC |
| 3151 | TCCAAGCCAC AAGATGACAG CGCTCAACAT CATGAAGGGA |
| | CGATACGGTT |
| 3201 | TTCTCGAGGG GTATCTAAAA TTCCTCAGAT TGCTGCTATA |
| | CAAGAGGGAA |
| 3251 | CCTTAGCTTT ATCACAAAAC GCAGAGCTTT GGTTGGCAGG |
| | ACTTAAACAG |
| 3301 | GAAACAGGAA GTTCTATCGT ATTGTCTGCG GGATCTATTC |
| | TCCGTATTTT |
| 3351 | TGATTCCCAG GTTGATAGCA GTGCGCCTCT TCCTACAGAA |
| | AATAAAGAGG |
| 3401 | AGACTCTTGT TTCTGCCGGA GTTCAAATTA ACATGAGCTC |
| | TCCTACACCC |
| 3451 | AATAAAGATA AAGCTGTAGA TACTCCAGTA CTTGCAGATA |
| | TCATAAGTAT |
| 3501 | TACTGTAGAT TTGTCTTCAT TTGTTCCTGA GCAAGACGGA |
| | ACTCTTCCTC |
| 3551 | TTCCTCCTGA AATTATCATT CCTAAGGGAA CAAAATTACA |
| | TTCTAATGCC |
| 3601 | ATAGATCTTA AGATTATAGA TCCTACCAAT GTGGGATATG |
| | AAAATCATGC |
| 3651 | TCTTCTAAGT TCTCATAAAG ATATTCCATT AATTTCTCTT |
| | AAGACAGCGG |
| 3701 | AAGGAATGAC AGGGACGCCT ACAGCAGATG CTTCTCTATC |
| | TAATATAAAA |
| 3751 | ATAGATGTAT CTTTACCTTC GATCACACCA GCAACGTATG |
| | GTCACACAGG |
| 3801 | AGTTTGGTCT GAAAGTAAAA TGGAAGATGG AAGACTTGTA |
| | GTCGGTTGGC |
| 3851 | AACCTACGGG ATATAAGTTA AATCCTGAGA AGCAAGGGGC |
| | TCTAGTTTTG |
| 3901 | AATAATCTCT GGAGTCATTA TACAGATCTT AGAGCTCTTA |
| | AGCAGGAGAT |
| 3951 | CTTTGCTCAT CATACGATAG CTCAAAGAAT GGAGTTAGAT |
| | TTCTCGACAA |
| 4001 | ATGTCTGGGG ATCAGGATTA GGTGTTGTTG AAGATTGTCA |
| | GAACATCGGA |
| 4051 | GAGTTTGATG GGTTCAAACA TCATCTCACA GGGTATGCCC |
| | TAGGCTTGGA |
| 4101 | TACACAACTA GTTGAAGACT TCTTAATTGG AGGATGTTTC |
| | TCACAGTTCT |
| 4151 | TTGGTAAAAC TGAAAGCCAA TCCTACAAAG CTAAGAACGA |
| | TGTGAAGAGT |
| 4201 | TATATGGGAG CTGCTTATGC GGGGATTTTA GCAGGTCCTT |
| | GGTTAATAAA |
| 4251 | AGGAGCTTTT GTTTACGGTA ATATAAACAA CGATTTGACT |
| | ACAGATTACG |
| 4301 | GTACTTTAGG TATTTCAACA GGTTCATGGA TAGGAAAAGG |
| | GTTTATCGCA |
| 4351 | GGCACAAGCA TTGATTACCG CTATATTGTA AATCCTCGAC |
| | GGTTTATATC |
| 4401 | GGCAATCGTA TCCACAGTGG TTCCTTTTGT AGAAGCCGAG |
| | TATGTCCGTA |
| 4451 | TAGATCTTCC AGAAATTAGC GAACAGGGTA AAGAGGTTAG |
| | AACGTTCCAA |
| 4501 | AAAACTCGTT TTGAGAATGT CGCCATTCCT TTTGGATTTG |
| | CTTTAGAACA |
| 4551 | TGCTTATTCG CGTGGCTCAC GTGCTGAAGT GAACAGTGTA |
| | CAGCTTGCTT |
| 4601 | ACGTCTTTGA TGTATATCGT AAGGGACCTG TCTCTTTGAT |
| | TACACTCAAG |
| 4651 | GATGCTGCTT ATTCTTGGAA GAGTTATGGG GTAGATATTC |
| | CTTGTAAAGC |
| 4701 | TTGGAAGGCT CGCTTGAGCA ATAATACGGA ATGGAATTCA |
| | TATTTAAGTA |
| 4751 | CGTATTTAGC GTTTAATTAT GAATGGAGAG AAGATCTGAT |
| | AGCTTATGAC |
| 4801 | TTCAATGGTG GTATCCGTAT TATTTTCTAG |

The PSORT algorithm predicts an inner membrane location (0.106).

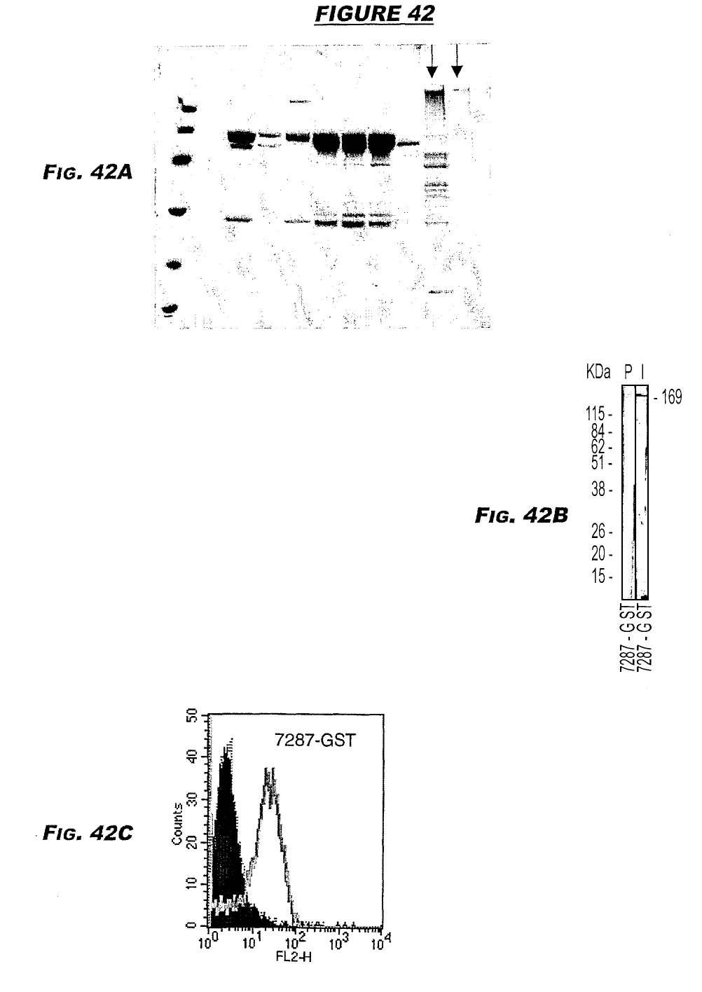

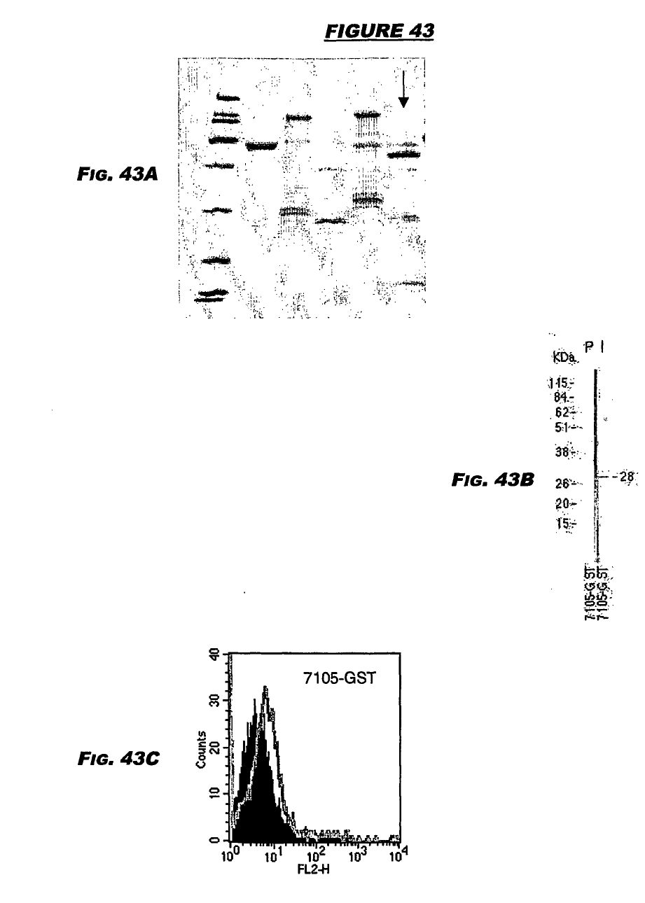

The protein was expressed in E. coli and purified as a GST-fusion product, as shown in FIG. 42A. The recombinant protein was used to immunise mice, whose sera were used in a Western blot (FIG. 42B) and for FACS analysis (FIG. 42C). A his-tagged protein was also expressed.

The cp7287 protein was also identified in the 2D-PAGE experiment and showed good cross-reactivity with human sera, including sera from patients with pneumonitis.

These experiments show that cp7287 is a surface-exposed and immunoaccessible protein, and that it is a useful immunogen. These properties are not evident from the sequence alone.

### Example 43

The following C. pneumoniae protein (PID 4377105) was expressed <SEQ ID 85; cp7105>:

| | |
|---|---|
| 1 | MSLYQKWWNS QLKKSLCYST VAALIFMIPS QESFADSLID |
| | LNLGLDPSVE |
| 51 | CLSGDGAFSV GYFTKAGSTP VEYQPFKYDV SKKTFTILSV |
| | ETANQSGYAY |
| 101 | GISYDGTITV GTCSLGAGKY NSAKWSADGT LTPLTGITGG |
| | TSHTEARAIS |
| 151 | KDTQVIEGFS YDASGQPKAV QWASGATTVT QLADISGGSR |
| | SSYAYAISDD |
| 201 | GTIIVGSMES TITRKTTAVK WVNNVPTYLG TLGGDASTGL |
| | YISGDGTVIV |
| 251 | GAANTATVTN GNQESHAYMY KDNQMKD* |

The cp7105 nucleotide sequence <SEQ ID 86> is:

| | |
|---|---|
| 1 | GTGAGTCTAT ATCAAAAATG GTGGAACAGT CAGTTAAAGA |
| | AGAGCCTCTG |
| 51 | CTATTCGACT GTTGCTGCTC TAATATTTAT GATTCCTTCT |
| | CAAGAATCCT |
| 101 | TTGCAGATAG TCTTATAGAT TTAAATTTAG GTTTAGATCC |
| | TTCGGTCGAA |
| 151 | TGTCTGTCAG GAGATGGTGC ATTTTCTGTT GGGTATTTTA |
| | CTAAGGCGGG |
| 201 | ATCGACTCCC GTAGAATATC AGCCGTTTAA ATACGACGTA |
| | TCTAAGAAGA |
| 251 | CATTCACAAT CCTTTCCGTA GAAACGGCAA ATCAGAGCGG |
| | CTATGCTTAC |
| 301 | GGAATCTCCT ACGATGGCAC GATCACTGTA GGAACGTGTA |
| | GCCTAGGTGC |
| 351 | AGGAAAATAT AACGGCGCAA AATGGAGTGC GGATGGCACT |
| | TTAACACCCT |
| 401 | TAACTGGAAT CACGGGGGGG ACGTCACATA CGGAAGCGCG |
| | TGCGATTTCT |
| 451 | AAGGATACTC AGGTGATCGA GGGTTTCTCA TATGATGCTT |
| | CAGGGCAACC |
| 501 | CAAGGCTGTG CAGTGGGCAA GCGGAGCGAC TACAGTAACA |
| | CAATTAGCAG |
| 551 | ATATTTCAGG AGGCTCTAGA AGCTCTTATG CGTATGCTAT |
| | ATCTGATGAT |
| 601 | GGCACGATTA TTGTTGGGTC TATGGAGAGC ACGATAACAA |
| | GGAAAACTAC |
| 651 | AGCTGTAAAA TGGGTAAATA ATGTTCCTAC GTATCTGGGA |
| | ACCTTAGGAG |
| 701 | GAGATGCTTC TACAGGTCTT TATATTTCTG GAGACGGCAC |
| | CGTGATTGTA |
| 751 | GGTGCGGCAA ATACAGCAAC TGTAACCAAT GGGAATCAGG |
| | AATCCCACGC |
| 801 | CTATATGTAT AAAGATAACC AAATGAAAGA TTGA |

The PSORT algorithm predicts an inner membrane location (0.100).

The protein was expressed in E. coli and purified as a GST-fusion product, as shown in FIG. 43A. The recombinant protein was used to immunise mice, whose sera were used in a Western blot (FIG. 43B) and for FACS analysis (FIG. 43C). A his-tagged protein was also expressed.

This protein also showed good cross-reactivity with human sera, including sera from patients with pneumonitis.

These experiments show that cp7105 is a surface-exposed and immunoaccessible protein, and that it is a useful immunogen. These properties are not evident from the sequence alone.

### Example 44

The following C. pneumoniae protein (PID 4376802) was expressed <SEQ ID 87; cp6802>:

| | |
|---|---|
| 1 | MSNQLQPCIS LGCVSYINSF PLSLQLIKRN DIRCVLAPPA |
| | DLLNLLIEGK |
| 51 | LDVALTSSLG AISHNLGYVP GFGIAANQRI LSVNLYAAPT |
| | FFNSPQPRIA |
| 101 | ATLFSRSSIG LLKVLCRHLW RIPTPHILRF ITTKVLRQTP |
| | ENYDGLLLIG |
| 151 | DAALQHPVLP GFVTYDLASG WYDLTKLPFV FALLLHSTSW |
| | KEHPLPNLAM |
| 201 | EEALQQFESS PEEVLKEAHQ HTGLPPSLLQ EYYALCQYRL |
| | GEEHYESFEK |
| 251 | FREYYGTLYQ QARL* |

A predicted signal peptide is highlighted.

The cp6802 nucleotide sequence <SEQ ID 88> is:

| | |
|---|---|
| 1 | ATGTCTAACC AACTCCAGCC ATGTATAAGC TTAGGCTGCG |
| | TAAGTTATAT |
| 51 | TAATTCCTTT CCGCTGTCCC TACAACTCAT AAAAAGAAAC |
| | GATATTCGCT |
| 101 | GTGTTCTTGC TCCCCCTGCA GACCTCCTCA ACTTGCTAAT |
| | CGAAGGGAAA |
| 151 | CTCGATGTTG CTTTGACCTC ATCCCTAGGA GCTATCTCTC |
| | ATAACTTGGG |
| 201 | GTATGTCCCC GGCTTTGGAA TTGCAGCAAA CCAACGTATC |
| | CTCAGTGTAA |
| 251 | ACCTCTATGC AGCTCCCACT TTCTTTAACT CACCGCAACC |
| | TCGGATTGCC |
| 301 | GCAACTTTAG AAAGTCGCTC CTCTATAGGA CTCTTAAAAG |
| | TGCTTTGTCG |
| 351 | TCATCTCTGG CGCATCCCAA CTCCTCATAT CCTAAGATTC |
| | ATAACTACAA |
| 401 | AAGTACTCAG ACAAACCCCT GAAAATTATG ATGGCCTCCT |
| | CCTAATCGGA |
| 451 | GATGCAGCGC TACAACATCC TGTACTTCCT GGATTTGTAA |
| | CCTATGACCT |
| 501 | TGCCTCGGGG TGGTATGATC TTACAAAGCT ACCTTTTGTA |
| | TTTGCTCTTC |
| 551 | TTCTACACAG CACCTCTTGG AAAGAACATC CCCTACCCAA |
| | CCTTGCGATG |
| 601 | GAAGAAGCCC TCCAACAGTT CGAATCTTCA CCCGAAGAAG |
| | TCCTTAAAGA |
| 651 | AGCTCATCAA CATACAGGTC TGCCCCCTTC TCTTCTTCAA |
| | GAATACTATG |
| 701 | CCCTATGCCA GTACCGTCTA GGAGAAGAAC ACTACGAAAG |
| | CTTTGAAAAA |
| 751 | TTCCGGGAAT ATTATGGAAC CCTCTACCAA CAAGCCCGAC |
| | TGTAA |

The PSORT algorithm predicts an inner membrane location (0.060).

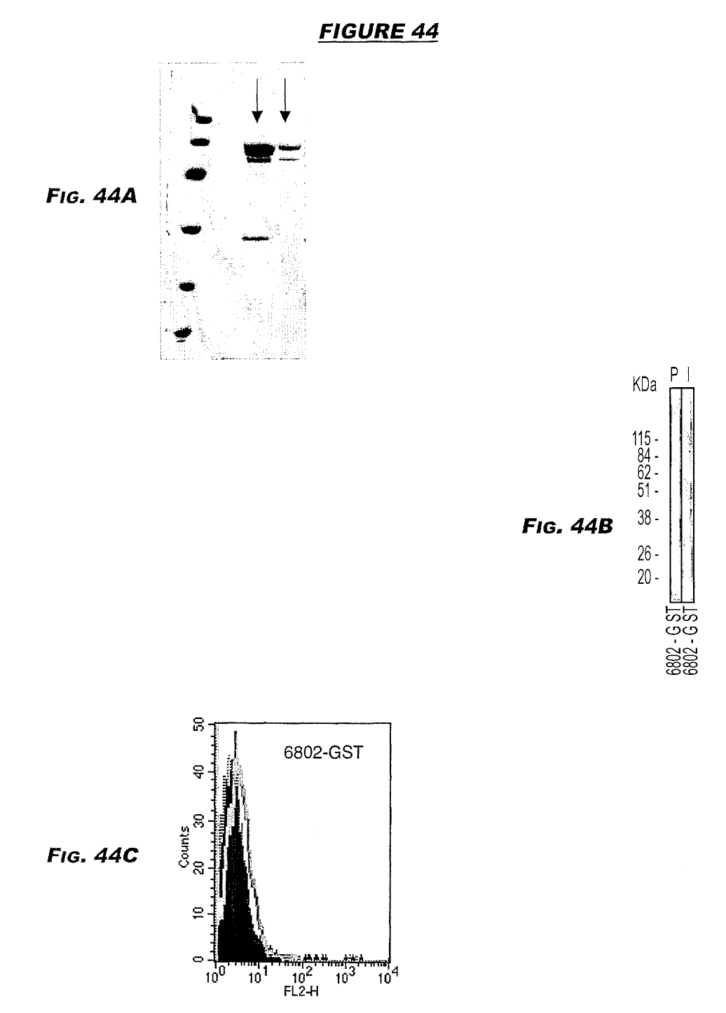

The protein was expressed in E. coli and purified as a GST-fusion product, as shown in FIG. 44A. The recombinant protein was used to immunise mice, whose sera were used in a Western blot (FIG. 44B) and for FACS analysis (FIG. 44C). A his-tagged protein was also expressed.

These experiments show that cp6802 is a surface-exposed and immunoaccessible protein, and that it is a useful immunogen. These properties are not evident from the sequence alone.

### Example 45

The following C. pneumoniae protein (PID 4376390) was expressed <SEQ ID 89; cp6390>:

| | |
|---|---|
| 1 | MVFSYYCMGL FFFSGAISSC GLLVSLGVGL GLSVLGVLLL |
| | LLAGLLLFKI |
| 51 | QSMLREVPKA PDLLDLEDAS ERLRVKASRS LASLPKEISQ |
| | LESYIRSAAN |
| 101 | DLNTIKTWPH KDQRLVETVS RKLERLAAAQ NYMISELCEI |
| | SEILEEEEHH |
| 151 | LILAQESLEW IGKSLFSTFL DMESFLNLSH LSEVRPYLAV |
| | NDPRLLEITE |
| 201 | ESWEVVSHFI NVTSAEKKAQ ILFKNNEHSR MKKKLESVQE |
| | LLETFIYKSL |
| 251 | KRSYRELGCL SEKMRIIHDN PLFPWVQDQQ KYAHAKNEFG |
| | EIARCLEEFE |
| 301 | KTFFWLDEEC AISYMDCWDF LNESIQNKKS RVDRDYISTK |
| | KIALKDRART |
| 351 | YAKVLLEENP TTEGKIDLQD AQRAFERQSQ EFYTLEHTET |
| | KVRLEALQQC |
| 401 | FSDLREATNV RQVRFTNSEN ANDLKESFEK IDKERVRYQK |
| | EQRLYWETID |
| 451 | RNEQELREEI GESLRLQNRR KGYRAGYDAG RLKGLLRQWK |
| | KNLRDVEAHL |
| 501 | EDATMDFEHE VSKSELCSVR ARLEVLEEEL MDMSPKVADI |
| | EELLSYEERC |
| 551 | ILPIRENLER AYLQYNKCSE ILSKAKFFFP EDEQLLVSEA |
| | NLREVGAQLK |
| 601 | QVQGKCQERA QKFAIFEKHI QEQKSLIKEQ VRSFDLAGVG |
| | FLKSELLSIA |
| 651 | CNLYIKAVVK ESIPVDVPCM QLYYSYYEDN EAVVRNRLLN |
| | MTERYQNFKR |
| 701 | SLNSIQFNGD VLLRDPVYQP EGHETRLKER ELQETTLSCK |
| | KLKVAQDRLS |
| 751 | ELESRLSRR |

A predicted signal peptide is highlighted.

The cp6390 nucleotide sequence <SEQ ID 90> is:

| | |
|---|---|
| 1 | TTGGTATTCT CATACTATTG CATGGGATTA TTTTTTTTCT |
| | CTGGAGCTAT |
| 51 | TTCTAGTTGT GGTCTTTTAG TGTCTCTAGG ACTTGGTTTA |
| | GGACTTAGTG |
| 101 | TTTTAGGAGT ACTTTTACTT CTCTTAGCAG GTCTTTTGCT |
| | TTTTAAGATC |
| 151 | CAAAGTATGC TTCGAGAGGT GCCTAAGGCT CCTGATCTAT |
| | TAGATTTAGA |
| 201 | AGATGCAAGT GAACGGCTTA GAGTAAAGGC TAGCCGTTCT |
| | TTAGCAAGCC |
| 251 | TCCCGAAGGA AATCAGTCAG CTAGAGAGCT ACATTCGTTC |
| | TGCAGCTAAT |
| 301 | GATCTAAATA CAATTAAGAC TTGGCCGCAT AAAGATCAAA |
| | GACTCGTCGA |
| 351 | GACCGTGTCA CGAAAATTAG AGCGTCTGGC AGCTGCTCAA |
| | AACTATATGA |
| 401 | TTTCTGAACT CTGCGAGATT AGTGAGATTC TTGAGGAAGA |
| | GGAGCATCAT |
| 451 | CTAATTTTGG CTCAGGAATC TCTAGAATGG ATAGGTAAGA |
| | GTCTATTTTC |
| 501 | TACCTTTCTG GACATGGAAT CTTTTTTAAA TTTGAGCCAT |
| | CTATCTGAAG |
| 551 | TGCGTCCGTA CTTAGCTGTA AATGATCCTA GATTATTAGA |
| | AATTACCGAA |
| 601 | GAATCTTGGG AAGTAGTGAG TCATTTCATA AATGTAACGT |
| | CTGCTTTTAA |
| 651 | GAAAGCTCAG ATTCTTTTTA AGAACAACGA ACATTCTCGG |
| | ATGAAGAAGA |
| 701 | AGTTAGAAAG TGTTCAAGAG TTACTGGAAA CATTTATTTA |
| | TAAGAGTTTA |
| 751 | AAGAGAAGTT ATCGAGAATT AGGATGCTTA AGTGAAAAGA |
| | TGAGAATCAT |
| 801 | TCACGACAAT CCTCTCTTCC CTTGGGTGCA AGATCAGCAG |
| | AAGTATGCTC |
| 851 | ATGCTAAGAA TGAATTTGGA GAGATTGCGC GGTGTTTAGA |
| | GGAGTTTGAA |
| 901 | AAGACGTTCT TCTGGTTGGA TGAGGAGTGT GCTATTTCTT |
| | ACATGGACTG |
| 951 | TTGGGATTTT CTAAATGAGT CTATTCAGAA TAAGAAGTCC |
| | AGAGTAGATC |
| 1001 | GAGATTATAT ATCCACGAAG AAAATTGCAT TAAAGGATAG |
| | AGCCCGCACT |
| 1051 | TATGCTAAGG TTCTTTTAGA AGAGAATCCG ACTACAGAGG |
| | GTAAAATAGA |
| 1101 | TTTGCAAGAC GCTCAAAGAG CCTTTGAGCG TCAAAGTCAG |
| | GAGTTTTATA |
| 1151 | CACTAGAGCA TACGGAAACA AAGGTGAGAC TAGAAGCACT |
| | TCAACAGTGC |
| 1201 | TTCTCGGATC TTAGGGAGGC GACGAACGTA AGGCAAGTTA |
| | GGTTTACAAA |
| 1251 | TTCTGAAAAT GCGAATGATT TAAAGGAGAG TTTCGAGAAG |
| | ATAGATAAAG |
| 1301 | AGCGTGTGCG ATATCAAAAA GAGCAAAGGC TCTATTGGGA |
| | AACAATAGAT |
| 1351 | CGCAATGAGC AAGAGCTTAG GGAAGAGATT GGGGAGTCGC |
| | TTCGTTTACA |
| 1401 | AAATCGGAGA AAAGGGTATA GGGCTGGATA TGATGCTGGG |
| | CGTTTAAAAG |
| 1451 | GTTTGTTGCG TCAGTGGAAG AAAAATCTCC GCGATGTGGA |
| | AGCCCACCTT |
| 1501 | GAAGATGCAA CTATGGATTT TGAGCATGAA GTAAGCAAGA |
| | GCGAATTGTG |
| 1551 | CAGTGTTCGG GCGAGGCTCG AGGTTCTAGA AGAAGAGCTG |
| | ATGGATATGT |
| 1601 | CTCCTAAAGT TGCGGATATA GAAGAGTTGT TGTCCTATGA |
| | AGAGCGTTGT |
| 1651 | ATTCTTCCTA TTAGGGAAAA TTTAGAAAGG GCATACCTCC |
| | AATATAATAA |
| 1701 | GTGTTCTGAA ATTTTATCCA AGGCAAAGTT CTTCTTTCCG |
| | GAAGACGAGC |
| 1751 | AATTGCTAGT TTCGGAAGCG AATCTAAGAG AGGTGGGTGC |
| | CCAGTTAAAA |
| 1801 | CAAGTACAGG GAAAATGTCA AGAGAGGGCC CAAAAGTTCG |
| | CAATATTTGA |
| 1851 | AAAGCATATT CAGGAGCAGA AAAGCCTTAT TAAAGAGCAA |
| | GTGCGGAGTT |
| 1901 | TTGATCTAGC GGGAGTTGGG TTTTTAAAGA GTGAGCTTCT |
| | TAGTATTGCT |
| 1951 | TGTAACCTTT ATATAAAGGC GGTTGTTAAG GAGTCTATAC |
| | CAGTTGATGT |
| 2001 | GCCTTGTATG CAGTTATATT ATAGTTATTA CGAAGATAAT |
| | GAAGCTGTAG |
| 2051 | TGCGAAACCG CCTTTTAAAT ATGACGGAGA GGTATCAAAA |
| | TTTTAAAAGG |
| 2101 | AGTTTGAATT CCATACAATT TAATGGTGAC GTTCTTTTAC |
| | GGGATCCGGT |
| 2151 | CTATCAACCT GAAGGTCATG AGACCAGGCT AAAGGAACGG |
| | GAGCTACAAG |
| 2201 | AAACAACTTT GTCTTGTAAG AAATTAAAAG TGGCTCAAGA |
| | TCGTCTTTCT |
| 2251 | GAATTAGAGT CAAGGCTGTC TAGGAGATAG |

The PSORT algorithm predicts a periplasmic location (0.932).

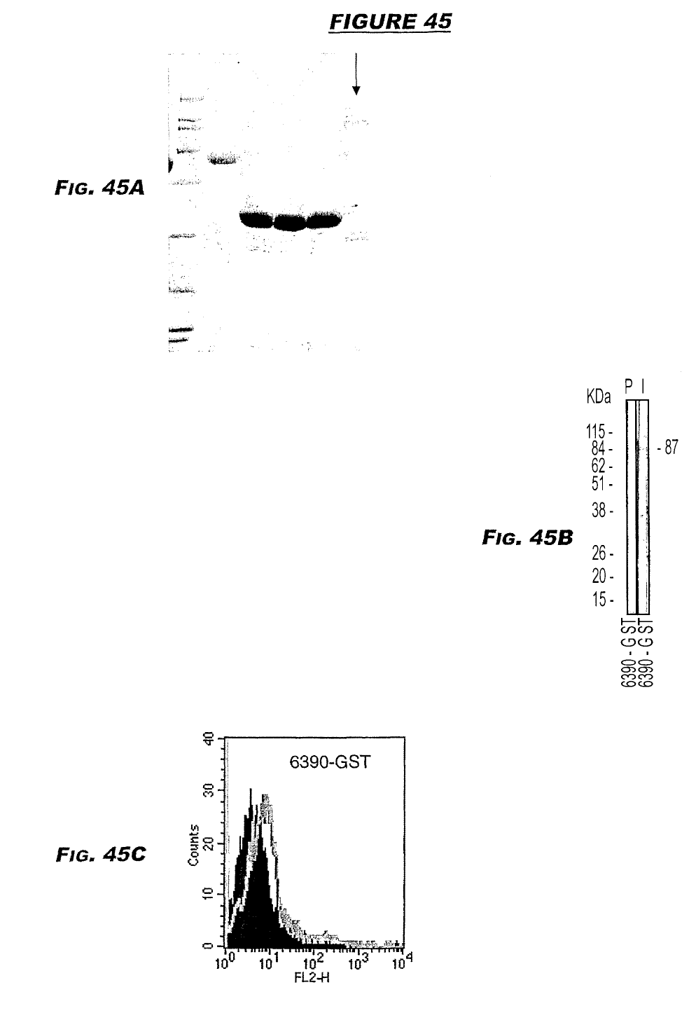

The protein was expressed in E. coli and purified as a GST-fusion product, as shown in FIG. 45A. The recombinant protein was used to immunise mice, whose sera were used in a Western blot (FIG. 45B) and for FACS analysis (FIG. 45C). A his-tagged protein was also expressed.

This protein also showed good cross-reactivity with human sera, including sera from patients with pneumonitis.

These experiments show that cp6390 is a surface-exposed and immunoaccessible protein, and that it is a useful immunogen. These properties are not evident from the sequence alone.

### Example 46

The following C. pneumoniae protein (PID 4376272) was expressed <SEQ ID 91; cp6272>:

| | |
|---|---|
| 1 | MKRCFLFLAS FVLMGSSADA LTHQEAVKKK NSYLSHFKSV |
| | SGIVTIEDGV |
| 51 | LNIHNNLRIQ ANKVYVENTV GQSLKLVAHG NVMVNYRAKT |
| | LVCDYLEYYE |
| 101 | DTDSCLLTNG RFAMYPWFLG GSMITLTPET IVIRKGYIST |
| | SEGPKKDLCL |
| 151 | SGDYLEYSSD SLLSIGKTTL RVCRIPILFL PPFSIMPMEI |
| | PKPPINFRGG |
| 201 | TGGFLGSYLG MSYSPISRKH FSSTFFLDSF FKHGVGMGFN |
| | LHCSQKQVPE |
| 251 | NVFNMKSYYA HRLAIDMAEA HDRYRLHGDF CFTHKHVNFS |
| | GEYHLSDSWE |
| 301 | TVADIFPNNF MLKNTGPTRV DCTWNDNYFE GYLTSSVKVN |
| | SFQNANQELP |
| 351 | YLTLRQYPIS IYNTGVYLEN IVECGYLNFA FSDHIVGENF |
| | SSLRLAARPK |
| 401 | LHKTVPLPIG TLSSTLGSSL IYYSDVPEIS SRHSQLSAKL |
| | QLDYRFLLHK |
| 451 | SYIQRRHIIE PFVTFITETR PLAKNEDHYI FSIQDAFHSL |
| | NLLKAGIDTS |
| 501 | VLSKTNPRFP RIHAKLWTTH ILSNTESKPT FPKTACELSL |
| | PFGKKNTVSL |
| 551 | DAEWIWKKHC WDHMNIRWEW IGNDNVAMTL ESLHRSKYSL |
| | IKCDRENFIL |
| 601 | DVSRPIDQLL DSPLSDHRNL ILGKDFVRPH PCWNYRLSLR |
| | YGWHRQDTPN |
| 651 | YLEYQMILGT KIFEHWQLYG VYERREADSR FFFFLKLDKP |
| | KKPPF* |

A predicted signal peptide is highlighted.

The cp6272 nucleotide sequence <SEQ ID 92> is:

| | |
|---|---|
| 1 | ATGAAACGTT GCTTCTTATT TCTAGCTTCC TTTGTTCTTA |
| | TGGGTTCCTC |
| 51 | AGCTGATGCT TTGACTCATC AAGAGGCTGT GAAAAAGAAA |
| | AACTCCTATC |
| 101 | TTAGTCACTT TAAGAGTGTT TCTGGGATTG TGACCATCGA |
| | AGATGGGGTA |
| 151 | TTGAATATCC ATAACAACCT GCGGATACAA GCCAATAAAG |
| | TGTATGTAGA |
| 201 | AAATACTGTG GGTCAAAGCC TGAAGCTTGT CGCACATGGC |
| | AATGTTATGG |
| 251 | TGAACTATAG GGCAAAAACC CTAGTTTGTG ATTACCTAGA |
| | GTATTACGAA |
| 301 | GATACAGACT CTTGTCTTCT TACTAATGGA AGATTCGCGA |
| | TGTATCCTTG |
| 351 | GTTTCTAGGG GGGTCTATGA TCACTCTAAC CCCAGAAACC |
| | ATAGTCATTC |
| 401 | GGAAGGGATA TATCTCTACC TCCGAGGGTC CCAAAAAAGA |
| | CCTGTGCCTC |
| 451 | TCCGGAGATT ACCTGGAATA TTCTTCAGAT AGTCTTCTTT |
| | CTATAGGGAA |
| 501 | GACAACATTA AGGGTGTGTC GCATTCCGAT ACTTTTCTTA |
| | CCTCCATTTT |
| 551 | CTATCATGCC TATGGAGATC CCTAAGCCTC CGATAAACTT |
| | TCGAGGAGGA |
| 601 | ACAGGAGGAT TTCTGGGATC CTATTTGGGG ATGAGCTACT |
| | CGCCGATTTC |
| 651 | TAGGAAGCAT TTCTCCTCGA CATTTTTCTT GGATAGCTTT |
| | TTCAAGCATG |
| 701 | GCGTCGGCAT GGGATTCAAC CTCCATTGTT CTCAGAAGCA |
| | GGTTCCTGAG |
| 751 | AATGTCTTCA ATATGAAAAG CTATTATGCC CACCGCCTTG |
| | CTATCGATAT |
| 801 | GGCAGAAGCT CATGATCGCT ATCGCCTACA CGGAGATTTC |
| | TGCTTCACGC |
| 851 | ATAAGCATGT AAATTTTTCT GGAGAATACC ATCTCAGCGA |
| | TAGTTGGGAA |
| 901 | ACTGTTGCTG ACATTTTCCC CAACAACTTC ATGTTGAAAA |
| | ATACAGGCCC |
| 951 | CACACGTGTC GATTGCACTT GGAATGACAA CTAAAAAGAA |
| | GGGTATCTCA |
| 1001 | CCTCTTCTGT TAAGGTAAAC TCTTTCCAAA ATGCCAACCA |
| | AGAGCTCCCT |
| 1051 | TATTTAACAT TAAGGCAGTA CCCGATTTCT ATTTATAATA |
| | CGGGAGTGTA |
| 1101 | CCTTGAAAAC ATCGTAGAAT GTGGGTATTT AAACTTTGCT |
| | TTTAGCGATC |
| 1151 | ATATCGTTGG CGAGAATTTC TCTTCACTAC GTCTTGCTGC |
| | GCGCCCTAAG |
| 1201 | CTCCATAAAA CTGTGCCTCT ACCTATAGGA ACGCTCTCCT |
| | CCACCCTAGG |
| 1251 | GAGTTCTCTG ATTTACTATA GCGATGTTCC TGAGATCTCC |
| | TCGCGCCATA |
| 1301 | GTCAGCTTTC CGCGAAGCTA CAACTTGATT ATCGCTTTCT |
| | ATTACATAAG |
| 1351 | TCCTACATTC AAAGACGCCA TATTATAGAG CCGTTCGTTA |
| | CCTTCATTAC |
| 1401 | AGAGACTCGT CCTCTAGCTA AGAATGAAGA TCATTATATC |
| | TTTTCTATTC |
| 1451 | AAGATGCCTT TCACTCCTTA AACCTTCTGA AAGCGGGTAT |
| | AGATACCTCG |
| 1501 | GTACTGAGTA AGACTAACCC TCGATTCCCG AGAATCCATG |
| | CGAAGCTGTG |
| 1551 | CAGTACCCAC ATCTTGAGCA ATACAGAAAG CAAACCCACG |
| | TTTCCCAAAA |
| 1601 | CTGCATGCGA GCTATCTCTA CCTTTTGGAA AGAAAAATAC |
| | AGTCTCCTTA |
| 1651 | GATGCTGAAT GGATTTGGAA AAAGCACTGT TGGGATCACA |
| | TGAACATACG |
| 1701 | TTGGGAGTGG ATCGGAAATG ACAATGTGGC TATGACTCTA |
| | GAATCCCTGC |
| 1751 | ATAGAAGCAA ATACAGCCTG ATTAAGTGTG ACAGGGAGAA |
| | CTTCATTTTA |
| 1801 | GATGTCAGCC GTCCCATTGA CCAGCTTTTA GACTCCCCTC |
| | TCTCTGATCA |
| 1851 | TAGGAATCTC ATTTTAGGGA AATTATTTGT ACGACCTCAT |
| | CCCTGTTGGA |
| 1901 | ATTACCGCTT ATCCTTACGC TATGGCTGGC ATCGCCAGGA |
| | CACTCCGAAC |
| 1951 | TACCTAGAAT ACCAGATGAT TCTAGGGACG AAGATCTTCG |
| | AACATTGGCA |
| 2001 | GCTCTATGGG GTGTATGAAC GCCGAGAAGC AGATAGTCGA |
| | TTTTTCTTCT |
| 2051 | TCTTAAAGCT CGACAAACCT AAAAAACCTC CCTTCTAA |

The PSORT algorithm predicts an outer membrane location (0.48).

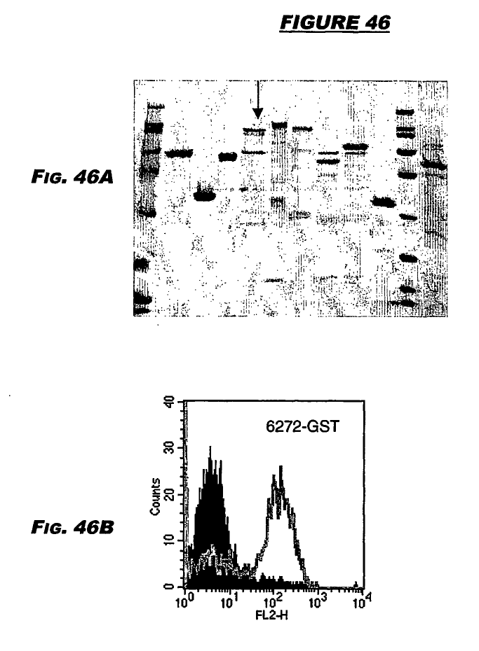

The protein was expressed in E. coli and purified as a GST-fusion product, as shown in FIG. 46A. The recombinant protein was used to immunise mice, whose sera were used in a Western blot and for FACS analysis (FIG. 46B). A his-tagged protein was also expressed.

This protein also showed good cross-reactivity with human sera, including sera from patients with pneumonitis.

These experiments show that cp6272 is a surface-exposed and immunoaccessible protein, and that it is a useful immunogen. These properties are not evident from the sequence alone.

### Example 47

The following C. pneumoniae protein (PID 4377111) was expressed <SEQ ID 93; cp7111>:

| | |
|---|---|
| 1 | MFEAVIADIQ AREILDSRGY PTLHVKVTTS TGSVGEARVP |
| | SGASTGKKEA |
| 51 | LEFRDTDSPR YQGKGVLQAV KNVKEILFPL VKGCSVYEQS |
| | LIDSLMMDSD |
| 101 | GSPNKETLGA NAILGVSLAT AHAAAATLRR PLYRYLGGCF |
| | ACSLPCPMMN |
| 151 | LINGGMHADN GLEFQEFMIR PIGASSIKEA VNMGADVFHT |
| | LKKLLHERGL |
| 201 | STGVGDEGGF APNLASNEEA LELLLLAIEK AGFTPGKDIS |
| | LALDCAASSF |
| 251 | YNVKTGTYDG RHYEEQIAIL SNLCDRYPID SIEDGLAEED |
| | YDGWALLTEV |
| 301 | LGEKVQIVGD DLFVTNPELI LEGISNGLAN SVLIKPNQIG |
| | TLTETVYAIK |
| 351 | LAWMAGYTTI ISHRSGETTD TTIADLAVAF NAGQIKTGSL |
| | SRSERVAKYN |
| 401 | RLMEIEEELG SEAIFTDSNV FSYEDSEE* |

A predicted signal peptide is highlighted.

The cp7111 nucleotide sequence <SEQ ID 94> is:

| | |
|---|---|
| 1 | ATGTTTGAAG CTGTCATTGC CGATATCCAG GCTAGGGAAA |
| | TCTTGGATTC |
| 51 | TCGCGGGTAT CCCACTTTAC ATGTTAAAGT AACCACTAGC |
| | ACAGGTTCTG |
| 101 | TTGGAGAAGC TCGGGTTCCT TCAGGAGCAT CCACAGGGAA |
| | AAAAGAAGCC |
| 151 | TTAGAGTTTC GTGATACAGA TTCTCCTCGT TATCAAGGCA |
| | AAGGGGTTTT |
| 201 | GCAAGCTGTA AAAAACGTAA AAGAAATTCT TTTTCCCCTC |
| | GTCAAGGGAT |
| 251 | GTAGTGTTTA TGAGCAATCC TTAATTGATT CTCTGATGAT |
| | GGATTCTGAC |
| 301 | GGCTCTCCGA ACAAAGAAAC TCTAGGGGCC AATGCTATTT |
| | TAGGAGTCTC |
| 351 | TCTAGCTACA GCACATGCAG CAGCAGCAAC ACTACGCAGA |
| | CCTCTGTATC |
| 401 | GTTATTTAGG AGGGTGTTTT GCCTGCAGTC TTCCCTGTCC |
| | TATGATGAAT |
| 451 | CTGATCAATG GAGGCATGCA TGCCGATAAC GGCTTGGAGT |
| | TCCAAGAATT |
| 501 | TATGATCCGT CCTATTGGAG CCTCTTCCAT CAAAGAAGCT |
| | GTCAACATGG |
| 551 | GTGCTGACGT TTTTCATACT TTGAAAAAAT TACTCCATGA |
| | AAGAGGCTTA |
| 601 | TCTACTGGAG TGGGTGACGA AGGAGGCTTC GCCCCGAATC |
| | TTGCTTCTAA |
| 651 | TGAAGAAGCT CTAGAGCTCC TATTGCTGGC TATTGAAAAA |
| | GCAGGCTTTA |
| 701 | CTCCAGGAAA AGATATATCG CTAGCCTTAG ACTGCGCAGC |
| | ATCCTCATTC |
| 751 | TATAACGTAA AAACAGGCAC GTATGATGGG AGGCACTATG |
| | AAGAGCAAAT |
| 801 | CGCAATCCTT TCTAATTTAT GTGATCGCTA TCCTATAGAC |
| | TCCATAGAAG |
| 851 | ATGGTCTTGC TGAAGAAGAC TATGACGGGT GGGCCTTGTT |
| | AACTGAAGTT |
| 901 | CTTGGAGAAA AAGTACAGAT TGTGGGTGAT GACCTATTTG |
| | TTACAAATCC |
| 951 | GGAATTAATA TTAGAGGGTA TTAGCAATGG ATTAGCGAAC |
| | TCTGTGTTGA |
| 1001 | TTAAACCAAA TCAGATAGGG ACGCTTACTG AAACAGTGTA |
| | TGCTATCAAG |
| 1051 | CTTGCGCAAA TGGCTGGCTA TACTACAATT ATTTCTCATC |
| | GCTCAGGAGA |
| 1101 | AACTACGGAC ACTACGATTG CAGATCTTGC TGTTGCCTTC |
| | AACGCCGGTC |
| 1151 | AAATCAAAAC AGGCTCTTTA TCACGTTCTG AGCGTGTTGC |
| | AAAATACAAT |
| 1201 | AGACTCATGG AAATTGAAGA AGAGCTTGGA TCCGAAGCAA |
| | TTTTCACAGA |
| 1251 | TTCTAATGTA TTTTCTTAC GAGGATTCT GAGGAATAG |

The PSORT algorithm predicts an inner membrane location (0.100).

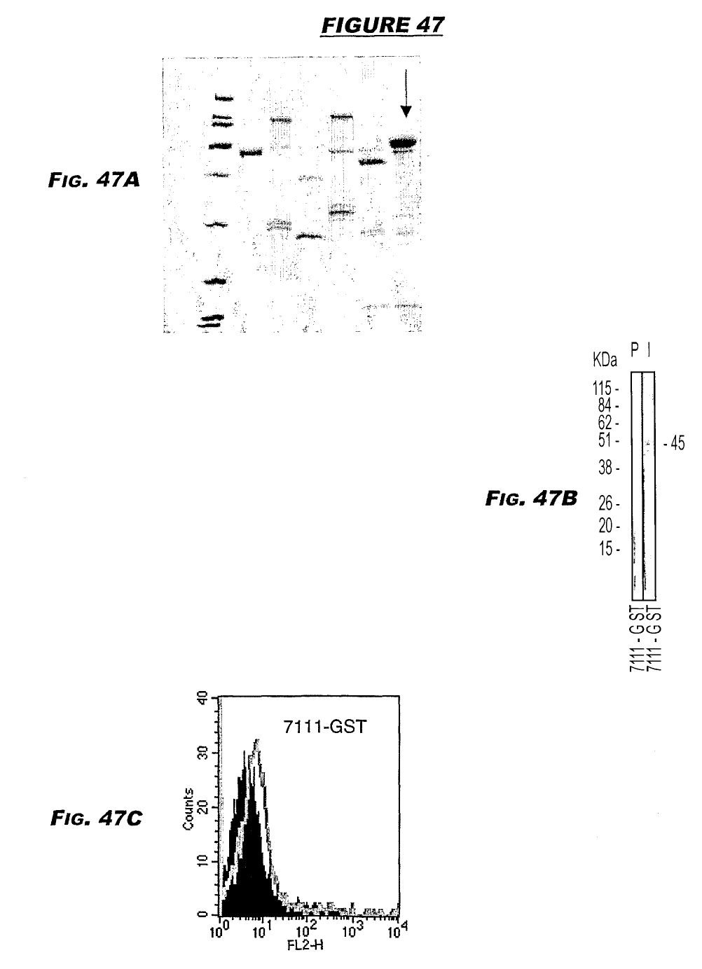

The protein was expressed in E. coli and purified as a GST-fusion product, as shown in FIG. 47A. The recombinant protein was used to immunise mice, whose sera were used in a Western blot (FIG. 47B) and for FACS analysis (FIG. 47C). A his-tagged protein was also expressed.

The cp7111 protein was also identified in the 2D-PAGE experiment and showed good cross-reactivity with human sera, including sera from patients with pneumonitis.

These experiments show that cp7111 is a surface-exposed and immunoaccessible protein, and that it is a useful immunogen. These properties are not evident from the sequence alone.

### Example 48

The following C. pneumoniae protein (PID 4455886) was expressed <SEQ ID 95; cp0010>:

| | |
|---|---|
| 1 | MKSQFSWLVL SSTLACFTSC STVFAATAEN IGPSDSFDGS |
| | TNTGTYTPKN |
| 51 | TTTGIDYTLT GDITLQNLGD SAALTKGCFS DTTESLSFAG |
| | KGYSLSFLNI |
| 101 | KSSAEGAALS VTTDKNLSLT GFSSLTFLAA PSSVITTPSG |
| | KGAVKCGGDL |
| 151 | TFDNNGTILF KQDYCEENGG AISTKNLSLK NSTGSISFEG |
| | NKSSATGKKG |
| 201 | GAICATGTVD ITNNTAPTLF SNNIAEAAGG AINSTGNCTI |
| | TGNTSLVFSE |
| 251 | NSVTATAGNG GALSGDADVT ISGNQSVTFS GNQAVANGGA |
| | IYAKKLTLAS |
| 301 | GGGGVSPFLT IIVQGTTAGN GGAISILAAG ECSLSAEAGD |
| | ITFNGNAIVA |
| 351 | TTPQTTKRNS IDIGSTAKIT NLRAISGHSI FFYDPITANT |
| | AADSTDTLNL |
| 401 | NKADAGNSTD YSGSIVFSGE KLSEDEAKVA DNLTSTLKQP |
| | VTLTAGNLVL |
| 451 | KRGVTLDTKG FTQTAGSSVI MDAGTTLKAS TEEVTLTGLS |
| | IPVDSLGEGK |
| 501 | KVVIAASAAS KNVALSGPIL LLDNQGNAYE NHDLGKTQDF |
| | SFVQLSALGT |
| 551 | ATTTDVPAVP TVATPTHYGY QGTWGMTWVD DTASTPKTKT |
| | ATLAWTNTGY |
| 601 | LPNPERQGPL VPNSLWGSFS DIQAIQGVIE RSALTLCSDR |
| | GFWAAGVANF |
| 651 | LDKDKKGEKR KYRHKSGGYA IGGAAQTCSE NLISFAFCQL |
| | FGSDKDFLVA |
| 701 | KNHTDTYAGA FYIQHITECS GFIGCLLDKL PGSWSHKPLV |
| | LEGQLAYSHV |
| 751 | SNDLKTKYTA YPEVKGSWGN NAFNMMLGAS SHSYPEYLHC |
| | FDTYAPYIKL |
| 801 | NLTYIRQDSF SEKGTEGRSF DDSNLFNLSL PIGVKFEKFS |
| | DCNDFSYDLT |
| 851 | LSYVPDLIRN DPKCTTALVI SGASWETYAN NLARQALQVR |
| | AGSHYAFSPM |
| 901 | FEVLGQFVFE VRGSSRIYNV DLGGKFQF* |

A predicted signal peptide is highlighted.

The cp0010 nucleotide sequence <SEQ ID 96> is:

| | |
|---|---|
| 1 | ATGAAATCGC AATTTTCCTG GTTAGTGCTC TCTTCGACAT |
| | TGGCATGTTT |
| 51 | TACTAGTTGT TCCACTGTTT TTGCTGCAAC TGCTGAAAAT |
| | ATAGGCCCCT |
| 101 | CTGATAGCTT TGACGGAAGT ACTAACACAG GCACCTATAC |
| | TCCTAAAAAT |
| 151 | ACGACTACTG GAATAGACTA TACTCTGACA GGAGATATAA |
| | CTCTGCAAAA |
| 201 | CCTTGGGGAT TCGGCAGCTT TAACGAAGGG TTGTTTTTCT |
| | GACACTACGG |
| 251 | AATCTTTAAG CTTTGCCGGT AAGGGGTACT CACTTTCTTT |
| | TTTAAATATT |
| 301 | AAGTCTAGTG CTGAAGGCGC AGCACTTTCT GTTACAACTG |
| | ATAAAAATCT |
| 351 | GTCGCTAACA GGATTTTCGA GTCTTACTTT CTTAGCGGCC |
| | CCATCATCGG |
| 401 | TAATCACAAC CCCCTCAGGA AAAGGTGCAG TTAAATGTGG |
| | AGGGGATCTT |
| 451 | ACATTTGATA ACAATGGAAC TATTTTATTT AAACAAGATT |
| | ACTGTGAGGA |
| 501 | AAATGGCGGA GCCATTTCTA CCAAGAATCT TTCTTTGAAA |
| | AACAGCACGG |
| 551 | GATCGATTTC TTTTGAAGGG AATAAATCGA GCGCAACAGG |
| | GAAAAAAGGT |
| 601 | GGGGCTATTT GTGCTACTGG TACTGTAGAT ATTACAAATA |
| | ATACGGCTCC |
| 651 | TACCCTCTTC TCGAACAATA TTGCTGAAGC TGCAGGTGGA |
| | GCTATAAATA |
| 701 | GCACAGGAAA CTGTACAATT ACAGGGAATA CGTCTCTTGT |
| | ATTTTCTGAA |
| 751 | AATAGTGTGA CAGCGACCGC AGGAAATGGA GGAGCTCTTT |
| | CTGGAGATGC |
| 801 | CGATGTTACC ATATCTGGGA ATCAGAGTGT AACTTTCTCA |
| | GGAAACCAAG |
| 851 | CTGTAGCTAA TGGCGGAGCC ATTTATGCTA AGAAGCTTAC |
| | ACTGGCTTCC |
| 901 | GGGGGGGGGG GGGTATCTCC TTTTCTAACA ATAaTAGTCC |
| | AAGGTACCAC |
| 951 | TGCAGGTAAT GGTGGAGCCA TTTCTATACT GGCAGCTGGA |
| | GAGTGTAGTC |
| 1001 | TTTCAGCAGA AGCAGGGGAC ATTACCTTCA ATGGGAATGC |
| | CATTGTTGCA |
| 1051 | ACTACACCAC AAACTACAAA AAGAAATTCT ATTGACATAG |
| | GATCTACTGC |
| 1101 | AAAGATCACG AATTTACGTG CAATATCTGG GCATAGCATC |
| | TTTTTCTACG |
| 1151 | ATCCGATTAC TGCTAATACG GCTGCGGATT CTACAGATAC |
| | TTTAAATCTC |
| 1201 | AATAAGGCTG ATGCAGGTAA TAGTACAGAT TATAGTGGGT |
| | CGATTGTTTT |
| 1251 | TTCTGGTGAA AAGCTCTCTG AAGATGAAGC AAAAGTTGCA |
| | GACAACCTCA |
| 1301 | CTTCTACGCT GAAGCAGCCT GTAACTCTAA CTGCAGGAAA |
| | TTTAGTACTT |
| 1351 | AAACGTGGTG TCACTCTCGA TACGAAAGGC TTTACTCAGA |
| | CCGCGGGTTC |
| 1401 | CTCTGTTATT ATGGATGCGG GCACAACGTT AAAAGCAAGT |
| | ACAGAGGAGG |
| 1451 | TCACTTTAAC AGGTCTCTCC ATTCCTGTAG ACTCTTTAGG |
| | CGAGGGTAAG |
| 1501 | AAAGTTGTAA TTGCTGCTTC TGCAGCAAGT AAAAATGTAG |
| | CCCTTAGTGG |
| 1551 | TCCGATTCTT CTTTTGGATA ACCAAGGGAA TGCTTATGAA |
| | AATCACGACT |
| 1601 | TAGGAAAAAC TCAAGACTTT TCATTTGTGC AGCTCTCTGC |
| | TCTGGGTACT |
| 1651 | GCAACAACTA CAGATGTTCC AGCGGTTCCT ACAGTAGCAA |
| | CTCCTACGCA |
| 1701 | CTATGGGTAT CAAGGTACTT GGGGAATGAC TTGGGTTGAT |
| | GATACCGCAA |
| 1751 | GCACTCCAAA GACTAAGACA GCGACATTAG CTTGGACCAA |
| | TACAGGCTAC |
| 1801 | CTTCCGAATC CTGAGCGTCA AGGACCTTTA GTTCCTAATA |
| | GCCTTTGGGG |
| 1851 | ATCTTTTTCA GACATCCAAG CGATTCAAGG TGTCATAGAG |
| | AGAAGTGCTT |
| 1901 | TGACTCTTTG TTCAGATCGA GGCTTCTGGG CTGCGGGAGT |
| | CGCCAATTTC |
| 1951 | TTAGATAAAG ATAAGAAAGG GGAAAAACGC AAATACCGTC |
| | ATAAATCTGG |
| 2001 | TGGATATGCT ATCGGAGGTG CAGCGCAAAC TTGTTCTGAA |
| | AACTTAATTA |
| 2051 | GCTTTGCCTT TTGCCAACTC TTTGGTAGCG ATAAAGATTT |
| | CTTAGTCGCT |
| 2101 | AAAAATCATA CTGATACCTA TGCAGGAGCC TTCTATATCC |
| | AACACATTAC |
| 2151 | AGAATGTAGT GGGTTCATAG GTTGTCTCTT AGATAAACTT |
| | CCTGGCTCTT |
| 2201 | GGAGTCATAA ACCCCTCGTT TTAGAAGGGC AGCTCGCTTA |
| | TAGCCACGTC |
| 2251 | AGTAATGATC TGAAGACAAA GTATACTGCG TATCCTGAGG |
| | TGAAAGGTTC |
| 2301 | TTGGGGGAAT AATGCTTTTA ACATGATGTT GGGAGCTTCT |
| | TCTCATTCTT |
| 2351 | ATCCTGAATA CCTGCATTGT TTTGATACCT ATGCTCCATA |
| | CATCAAACTG |
| 2401 | AATCTGACCT ATATACGTCA GGACAGCTTC TCGGAGAAAG |
| | GTACAGAAGG |
| 2451 | AAGATCTTTT GATGACAGCA ACCTCTTCAA TTTATCTTTG |
| | CCTATAGGGG |
| 2501 | TGAAGTTTGA GAAGTTCTCT GATTGTAATG ACTTTTCTTA |
| | TGATCTGACT |
| 2551 | TTATCCTATG TTCCTGATCT TATCCGCAAT GATCCCAAAT |
| | GCACTACAGC |
| 2601 | ACTTGTAATC AGCGGAGCCT CTTGGGAAAC TTATGCCAAT |
| | AACTTAGCAC |
| 2651 | GACAGGCCTT GCAAGTGCGT GCAGGCAGTC ACTACGCCTT |
| | CTCTCCTATG |
| 2701 | TTTGAAGTGC TCGGCCAGTT TGTCTTTGAA GTTCGTGGAT |
| | CCTCACGGAT |
| 2751 | TTATAATGTA GATCTTGGGG GTAAGTTCCA ATTCTAG |

The PSORT algorithm predicts an outer membrane location (0.922).

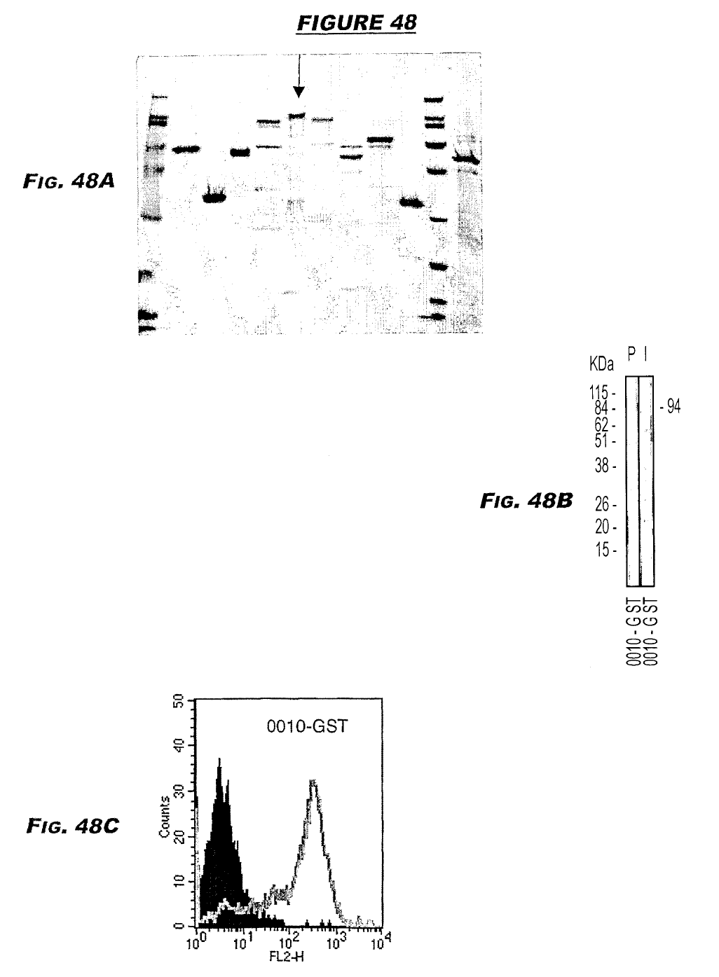

The protein was expressed in E. coli and purified as a GST-fusion product, as shown in FIG. 48A. The recombinant protein was used to immunise mice, whose sera were used in a Western blot (FIG. 48B) and for FACS analysis (FIG. 48C). A his-tagged protein was also expressed.

The cp0010 protein was also identified in the 2D-PAGE experiment and showed good cross-reactivity with human sera, including sera from patients with pneumonitis.

These experiments show that cp0010 is a surface-exposed and immunoaccessible protein, and that it is a useful immunogen. These properties are not evident from the sequence alone.

### Example 49

The following C. pneumoniae protein (PID 4376296) was expressed <SEQ ID 97; cp6296>:

| | |
|---|---|
| 1 | MEEVSEYLQQ VENQLESCSK RLTKMETFAL GVRLEAKEEI |
| | ESIILSDVVN |
| 51 | RFEVLCRDIE DMLSRVEEIE RMLRMAELPL LPIKEALTKA |
| | FVQHNSCKEK |
| 101 | LTKVEPYFKE SPAYLTSEER LQSLNQTLQR AYKESQKVSG |
| | LESEVRACRE |
| 151 | QLKDQVRQFE TQGVSLIKEE ILFVTSTFRT KFSYHSFRLH |
| | VPCMRLYEEY |
| 201 | YDDIDLERTR ARWMAMSERY RDAFQAFQEM LKEGLVEEAQ |
| | ALRETEYWLY |
| 251 | REERKSKKKH* |

The cp6296 nucleotide sequence <SEQ ID 98> is:

| | |
|---|---|
| 1 | ATGGAGGAGG TGTCTGAGTA TCTTCAGCAA GTAGAAAATC |
| | AGTTGGAATC |
| 51 | CTGTTCCAAG CGATTAACCA AGATGGAAAC TTTTGCCTTA |
| | GGTGTGAGGT |
| 101 | TGGAAGCTAA AGAAGAGATA GAGTCTATCA TACTTTCTGA |
| | TGTAGTGAAC |
| 151 | CGTTTTGAGG TTTTATGTAG AGATATTGAA GATATGCTAT |
| | CTCGAGTCGA |
| 201 | GGAGATAGAG CGGATGTTAC GTATGGCGGA GCTTCCTCTA |
| | CTTCCTATAA |
| 251 | AAGAAGCGCT TACCAAGGCT TTTGTACAAC ATAACAGCTG |
| | TAAAGAGAAG |
| 301 | TTAACCAAGG TAGAGCCTTA CTTTAAAGAG AGCCCTGCAT |
| | ATCTAACTAG |
| 351 | TGAAGAGCGA TTGCAGAGTT TGAATCAGAC TTTACAACGT |
| | GCGTACAAAG |
| 401 | AGTCCCAAAA GGTTTCAGGT TTAGAATCGG AAGTGAGAGC |
| | CTGTCGAGAG |
| 451 | CAGCTTAAAG ATCAAGTAAG ACAGTTTGAA ACTCAAGGAG |
| | TGAGCTTGAT |
| 501 | AAAAGAAGAG ATTCTCTTTG TGACTAGTAC CTTTAGAACT |
| | AAATTTAGCT |
| 551 | ATCATTCATT TCGATTACAT GTTCCTTGCA TGAGGTTGTA |
| | TGAGGAGTAT |
| 601 | TATGATGACA TTGATCTAGA GAGAACTCGA GCTCGATGGA |
| | TGGCGATGTC |
| 651 | TGAGAGGTAT AGAGATGCTT TTCAGGCATT CCAGGAGATG |
| | TTGAAGGAAG |
| 701 | GCCTAGTTGA AGAAGCTCAG GCTCTTAGAG AAACCGAGTA |
| | CTGGTTATAT |
| 751 | CGAGAGGAGA GAAAGAGTAA AAAGAAACAT TGA |

The PSORT algorithm predicts a cytoplasmic location (0.523).

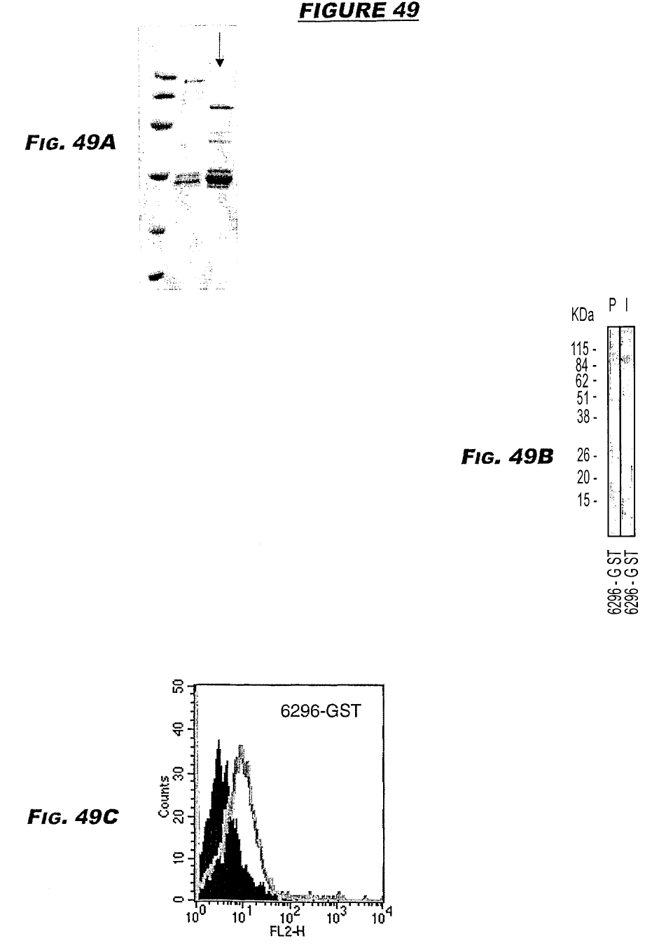

The protein was expressed in E. coli and purified as a GST-fusion product, as shown in FIG. 49A. The recombinant protein was used to immunise mice, whose sera were used in a Western blot (FIG. 49B) and for FACS analysis (FIG. 49C). A his-tagged protein was also expressed.

These experiments show that cp6296 is a surface-exposed and immunoaccessible protein, and that it is a useful immunogen. These properties are not evident from the sequence alone.

### Example 50

The following C. pneumoniae protein (PID 4376664) was expressed <SEQ ID 99; cp6664>:

| | |
|---|---|
| 1 | MVLFHAQASG RNRVKADAIV LPFWHFKDAK NAASFEAEFE |
| | PSYLPALENF |
| 51 | QGKIGEIELL YSSPKAKEKR IVLLGLGKNE ELTSDVVFQT |
| | YATLTRVLRK |
| 101 | AKCSTVNIIL PTISELRLSA EEFLVGLSSG ILSLNYDYPR |
| | YNKVDRNLET |
| 151 | PLSKVTVIGI VPKMADAIFR KEAAIFEGVY LTRDLVNRNA |
| | DEITPKKLAE |
| 201 | VALNLGKEFP SIDTKVLGKD AIAKEKMGLL LAVSKGSCVD |
| | PHFIVVRYQG |
| 251 | RPKSKDHTVL IGKGVTFDSG GLDLKPGKSM LTMKEDMAGG |
| | ATVLGILSAL |
| 301 | AVLELPINVT GIIPATENAI DGASYKMGDV YVGMSGLSVE |
| | ICSTDAEGRL |
| 351 | ILADAITYAL KYCKPTRIID FATLTGAMVV SLGEEVAGFF |
| | SNNDVLAEDL |
| 401 | LEASAETSEP LWRLPLVKKY DKTLHSDIAD MKNLGSNRAG |
| | AITAALFLQR |
| 451 | FLEESSVAWA HLDIAGTAYH EKEEDRYPKY ASGFGVRSIL |
| | YYLENSLSK* |

The cp6664 nucleotide sequence <SEQ ID 100> is:

| | |
|---|---|
| 1 | GTGGTTTTAT TTCATGCTCA AGCCTCTGGG GCTAATCGTG |
| | TTAAGGCAGA |
| 51 | TGCTATACTC CTGCCCTTTT GGCATTTTAA GGATGCAAAA |
| | AATGCAGCTT |
| 101 | CTTTTGAAGC CGAGTTTGAA CCCTCGTATC TCCCCGCTTT |
| | AGAAAACTTT |
| 151 | CAAGGAAAAA CCGGGGAGAT TGAACTCCTT TATAGTAGTC |
| | CTAAAGCTAA |
| 201 | GGAAAAACGC ATTGTCCTCT TAGCTTAAGG GAAAAATGAA |
| | GAGCTCACCT |
| 251 | CTGATGTTGT TTTCCAAACC TATGCGACAC TAACTCGTGT |
| | CTTACGTAAA |
| 301 | GCAAAGTGTT CCACAGTCAA TATCATCTTA CCTACAATTT |
| | CTGAATTGCG |
| 351 | GCTTTCTGCC GAAGAATTCT TAGTGGGGTT GTCCTCAGGA |
| | ATTTTGTCAT |
| 401 | TAAACTATGA CTACCCACGT TATAATAAGG TAGATCGTAA |
| | TCTTGAAACT |
| 451 | CCTCTTTCTA AAGTCACGGT TATCGGTATC GTTCCCAAAA |
| | TGGCGGATGC |
| 501 | TATCTTTAGG AAAGAAGCAG CCATTTTCGA AGGCGTATAT |
| | CTCACTCGAG |
| 551 | ATCTTGTGAA CAGGAATGCT GATGAAATTA CCCCTAAGAA |
| | ATTGGCAGAG |
| 601 | GTTGCTCTGA ATCTGGGAAA AGAGTTCCCT AGTATTGATA |
| | CTAAGGTCTT |
| 651 | GGGAAAAGAT GCCATCGCCA AAGAGAAAAT GGGACTCCTA |
| | TTGGCTGTTT |
| 701 | CCAAGGGTTC TTGTGTGGAT CCACACTTTA TCGTTGTCCG |
| | TTATCAAGGA |
| 751 | CGTCCTAAGT CTAAAGATCA CACCGTCTTG ATAGGGAAAG |
| | GGGTCACTTT |
| 801 | TGACTCTGGA GGTTTAGACC TCAAGCCTGG AAAATCCATG |
| | CTTACTATGA |
| 851 | AAGAAGACAT GGCAGGTGGG GCTACAGTCC TCGGGATTCT |
| | CTCGGCGTTA |
| 901 | GCAGTTTTAG AGCTTCCTAT AAATGTCACG GGGATCATTC |
| | CTGCTACAGA |
| 951 | GAATGCTATC GATGGCGCCT CCTATAAAAT GGGAGATGTC |
| | TATGTAGGAA |
| 1001 | TGTCGGGGCT TTCTGTTGAG ATTTGTAGTA CCGATGCTGA |
| | GGGACGTCTT |
| 1051 | ATCCTCGCTG ATGCGATTAC ATATGCTTTA AAATATTGTA |
| | AACCGACACG |
| 1101 | TATTATAGAT TTTGCAACTC TAACAGGAGC TATGGTAGTC |
| | TCTCTAGGAG |
| 1151 | AAGAGGTTGC AGGTTTCTTT TCCAATAACG ATGTTTTAGC |
| | TGAAGATCTT |
| 1201 | TTAGAGGCGT CAGCCGAAAC CTCCGAGCCG TTATGGAGAC |
| | TTCCTCTAGT |
| 1251 | TAAGAAGTAT GATAAAACAT TGCATTCTGA TATTGCTGAT |
| | ATGAAAAATC |
| 1301 | TAGGCAGTAA CCGTGCAGGG GCTATTACAG CAGCATTATT |
| | CTTGCAGAGA |
| 1351 | TTTTTGGAAG AATCTTCGGT AGCTTGGGCA CATCTTGATA |
| | TTGCAGGTAC |
| 1401 | TGCATATCAT GAAAAAGAAG AAGACCGTTA TCCAAAATAT |
| | GCTTCAGGTT |
| 1451 | TTGGTGTTCG TTCTATTCTT TATTACTTAG AAAATAGTCT |
| | TTCTAAGTAG |

The PSORT algorithm predicts an inner membrane location (0.268).

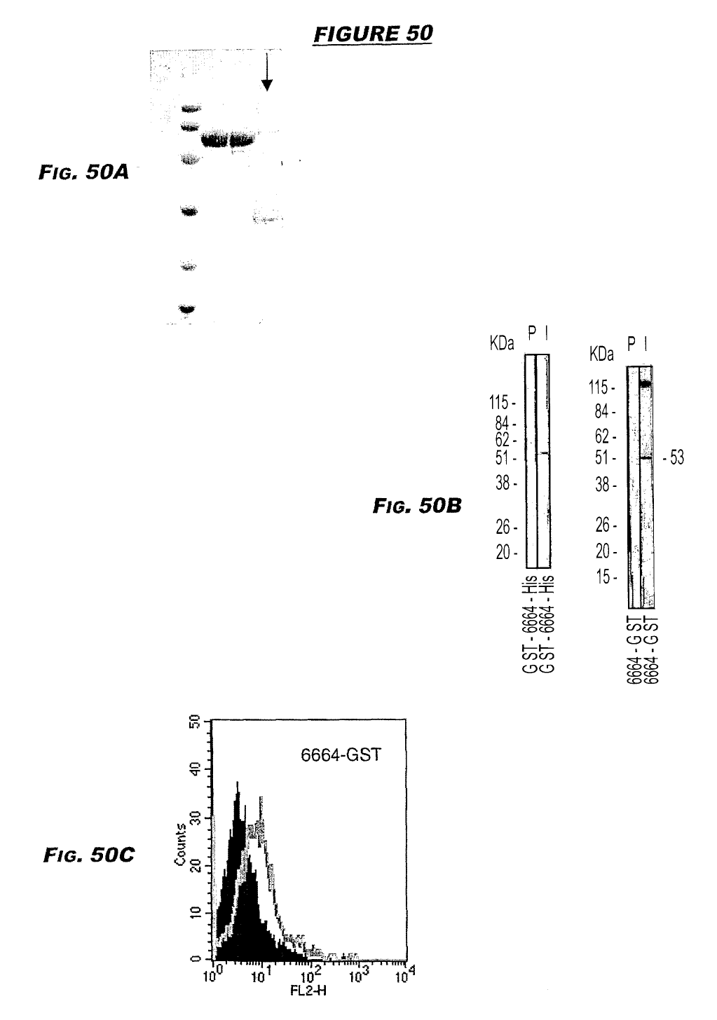

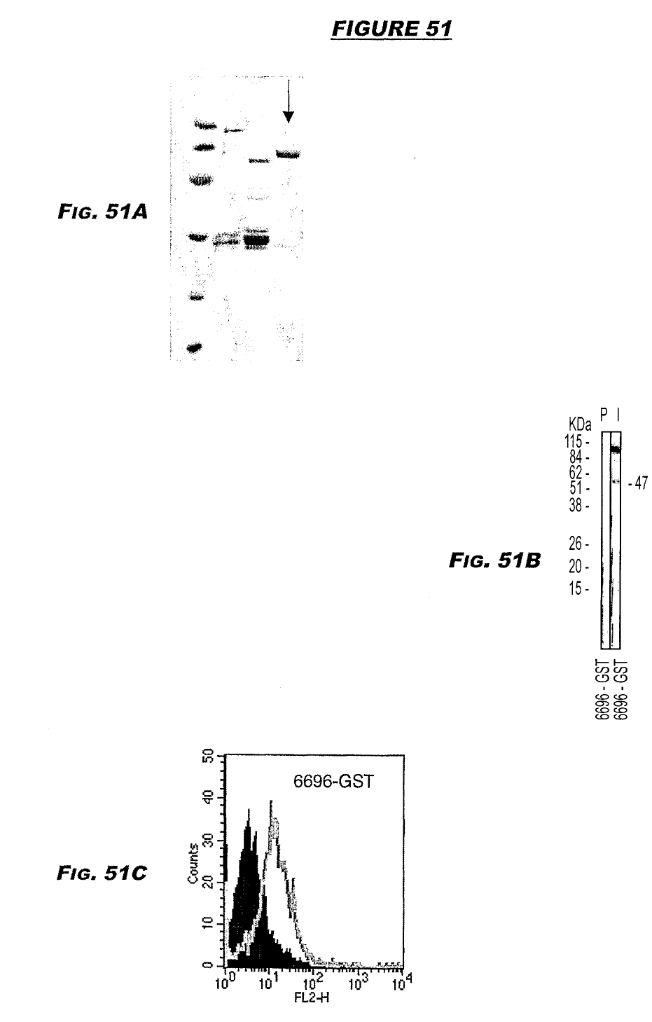

The protein was expressed in E. coli and purified as a GST-fusion (FIG. 50A), as a his-tagged protein, and as a GST/His fusion. The proteins were used to immunise mice, whose sera were used in Western blot Western blot (50B) and FACS (50C) analyses.

The cp6664 protein was also identified in the 2D-PAGE experiment (Cpn0385) and showed good cross-reactivity with human sera, including sera from patients with pneumonitis.

These experiments show that cp6664 is a surface-exposed and immunoaccessible protein, and that it is a useful immunogen. These properties are not evident from the sequence alone.

### Example 51

The following C. pneumoniae protein (PID 4376696) was expressed <SEQ ID 101; cp6696>:

| | |
|---|---|
| 1 | MTLIFVIIIV WCNAFLIKLC VIMGLQSRLQ HCIEVSQNSN |
| | FDSQVKQFIY |
| 51 | ACQDKTLRQS VLKIFRYHPL LKIHDIARAV YLLMALEEGE |
| | DLGLSFLNVQ |
| 101 | QYPSGAVELF SCGGFPWKGL PYPAEHAEFG LLLLQIAEFY |
| | EESQAYVSKM |
| 151 | SHFQQALFDH QGSVFPSLWS QENSRLLKEK TTLSQSFLFQ |
| | LGMQIHPEYS |
| 201 | LEDPALGFWM QRTRSSSAFV AASGCQSSLG AYSSGDVGVI |
| | AYGPCSGDIS |
| 251 | DCYYFGCCGI AKEFVCQKSH QTTEISFLTS TGKPHPRNTG |
| | FSYLRDSYVH |
| 301 | LPIRCKITIS DKQYRVHAAL AEATSAMTFS IFCKGKNCQV |
| | VDGPRLRSCS |
| 351 | LDSYKGPGND IMILGENDAI NIVSASPYME IFALQGKEKF |
| | WNADFLINIP |
| 401 | YKEEGVMIIF EKKVTSEKSR FFTKMN* |

A predicted signal peptide is highlighted.

The cp6696 nucleotide sequence <SEQ ID 102> is:

| | |
|---|---|
| 1 | TTGACTCTAA TTTTTGTTAT TATTATCGTT TGGTGCAATG |
| | CTTTTCTGAT |
| 51 | CAAATTGTGC GTGATAATGG GGCTGCAATC CAGGTTACAA |
| | CATTGTATAG |
| 101 | AAGTGTCCCA GAATTCGAAC TTTGATTCAC AAGTAAAACA |
| | GTTTATCTAT |
| 151 | GCGTGCCAAG ATAAGACATT AAGGCAGTCT GTACTCAAGA |
| | TTTTCCGCTA |
| 201 | CCATCCTTTA CTAAAAATTC ATGATATTGC TCGGGCCGTC |
| | TATCTTTTGA |
| 251 | TGGCCTTAGA AGAAGGCGAG GATTTAGGCT TAAGCTTTTT |
| | AAATGTACAG |
| 301 | CAGTACCCTT CAGGTGCTGT AGAACTGTTT TCTTGTGGGG |
| | GATTTCCTTG |
| 351 | GAAAGGATTA CCTTATCCTG CAGAACATGC GGAATTTGGC |
| | CTACTCCTGT |
| 401 | TACAGATCGC AGAGTTTTAT GAAGAGAGTC AGGCATACGT |
| | CTCTAAAATG |
| 451 | AGTCATTTTC AACAGGCACT CTTTGATCAC CAAGGGAGCG |
| | TCTTTCCCTC |
| 501 | TCTCTGGAGC CAGGAGAACT CTCGACTCCT AAAAGAAAAG |
| | ACAACTCTTA |
| 551 | GCCAATCGTT TCTCTTCCAA TTAGGAATGC AAATTCACCC |
| | AGAATACAGT |
| 601 | CTTGAGGATC CTGCACTAGG GTTCTGGATG CAAAGAACGC |
| | GTTCTTCATC |
| 651 | CGCTTTTGTA GCCGCTTCAG GATGTCAAAG TAGCTTGGGA |
| | GCGTATTCCT |
| 701 | CAGGGGATGT CGGTGTTATC GCTTATGGAC CTTGCTCTGG |
| | AGACATTAGT |
| 751 | GATTGTTATT ATTTTGGATG TTGTGGAATC GCTAAAGAGT |
| | TCGTGTGCCA |
| 801 | AAAATCTCAC CAAACTACAG AGATTTCTTT TCTCACCTCT |
| | ACAGGAAAGC |
| 851 | CTCATCCCAG AAATACGGGA TTTTCCTACC TTCGAGATTC |
| | CTATGTACAT |
| 901 | CTGCCGATCC GCTGTAAGAT CACTATTTCC GACAAGCAAT |
| | ATCGCGTGCA |
| 951 | CGCTGCGTTG GCTGAGGCCA CCTCTGCCAT GACGTTTTCT |
| | ATTTTCTGTA |
| 1001 | AGGGGAAGAA TTGTCAGGTT GTTGACGGCC CTCGCTTGCG |
| | CTCCTGTTCC |
| 1051 | CTAGATTCTT ATAAAGGTCC CGGAAACGAC ATTATGATTC |
| | TTGGGGAAAA |
| 1101 | TGACGCAATC AACATTGTTT CTGCAAGTCC CTATATGGAA |
| | ATTTTTGCTT |
| 1151 | TGCAAGGCAA AGAAAAATTT TGGAATGCAG ACTTTTTGAT |
| | TAATATTCCT |
| 1201 | TACAAAGAAG AGGGCGTCAT GTTAATTTTT GAAAAAAAAG |
| | TGACCTCTGA |
| 1251 | GAAAGGAAGA TTCTTTACGA AGATGAATTA A |

The PSORT algorithm predicts an inner membrane location (0.463).

The protein was expressed in E. coli and purified as a GST-fusion product, as shown in FIG. 51A. The recombinant protein was used to immunise mice, whose sera were used in a Western blot (FIG. 51B) and for FACS analysis (FIG. 51C). A his-tagged protein was also expressed.

This protein also showed good cross-reactivity with human sera, including sera from patients with pneumonitis.

These experiments show that cp6696 is a surface-exposed and immunoaccessible protein, and that it is a useful immunogen. These properties are not evident from the sequence alone.

### Example 52

The following C. pneumoniae protein (PID 4376790) was expressed <SEQ ID 103; cp6790>:

| | |
|---|---|
| 1 | MSEHKKSSKI IGIDIGTTNS CVSVMEGGQA KVITSSEGTR |
| | TTPSIVAFKG |
| 51 | NEKLVGIPAK RQAVTNPEKT LGSTKRFIGR KYSEVASEIQ |
| | TVPYTVTSGS |
| 101 | KGDAVFEVDG KQYTPEEIGA QILMKMKETA EAYLGETVTE |
| | AVITVPAYFN |
| 151 | DSQRASTKDA GRIAGLDVKR IIPEPTAAAL AYGIDKVGDK |
| | KIAVFDLGGG |
| 201 | TFDISILEIG DGVEEVLSTN GDTLLGGDDF DEVIIKWMIE |
| | EFKKQEGIDL |
| 251 | SKDNMALQRL KDAAEKAKIE LSGVSSTEIN QPFITMDAQG |
| | PKHLALTLTR |
| 301 | AQFEKLAASL IERTKSPCIK ALSDAKLSAK DIDDVLLVGG |
| | MSRMPAVQET |
| 351 | VKELFGKEPN KGVNPDEVVA IGAAIQGGVL GGEVKDVLLL |
| | DVIPLSLGIE |
| 401 | TLGGVMTTLV ERNTTIPTQK KQIFSTAADN QPAVTIVVLQ |
| | GERPMAKDNK |
| 451 | EIGRFDLTDI PPAPRGHPQI EVSFDIDANG IFHVSAKDVA |
| | SGKEQKIRIE |
| 501 | ASSGLQEDEI QRMVRDAEIN KEEDKKRREA SDAKNEADSM |
| | IFRAFKAIKD |
| 551 | YKEQIPETLV KEIEERIENV RNALKDDAPI EKIKEVTEDL |
| | SKHMQKIGES |
| 601 | MQSQSASAAA SSAANAKGGP NINTEDLKKH SFSTKPPSNN |
| | GSSEDHIEEA |
| 651 | DVEIIDNDDK* |

The cp6790 nucleotide sequence <SEQ ID 104> is:

| | |
|---|---|
| 1 | ATGAGTGAAC ACAAAAAATC AAGCAAAATT ATAGGTATAG |
| | ACTTAGGCAC |
| 51 | AACAAACTCC TGCGTATCTG TTATGGAAGG AGGACAAGCT |
| | AAAGTAATTA |
| 101 | CATCATCCGA AGGAACAAGA ACCACGCCAT CGATCGTTGC |
| | CTTCAAAGGT |
| 151 | AATGAGAAAT TAGTGGGGAT TCCAGCAAAA CGTCAAGCAG |
| | TGACAAATCC |
| 201 | AGAAAAAACT CTCGGCTCTA CAAAACGCTT TATTGGCCGT |
| | AAGTACTCTG |
| 251 | AAGTAGCTTC GGAAATCCAA ACCGTTCCTT ATACAGTCAC |
| | CTCCGGATCT |
| 301 | AAAGGTGATG CCGTTTTCGA AGTTGATGGC AAACAATACA |
| | CTCCAGAAGA |
| 351 | AATTGGCGCA CAAATCTTAA TGAAAATGAA AGAGACAGCA |
| | GAAGCTTATC |
| 401 | TAGGCGAAAC TGTCACAGAA GCAGTGATCA CCGTCCCCGC |
| | ATACTTCAAT |
| 451 | GATTCTCAAC GAGCATCCAC AAAAGATGCT GGACGCATTG |
| | CAGGTCTAGA |
| 501 | TGTAAAACGT ATCATTCCAG AACCTACCGC AGCAGCTCTT |
| | GCCTACGGAA |
| 551 | TCGATAAAGT CGGTGATAAA AAAATCGCTG TCTTCGACCT |
| | TGGTGGAGGA |
| 601 | ACTTTTGATA TCTCCATCCT AGAAATCGGT GATGGCGTCT |
| | TCGAAGTTCT |
| 651 | ATCTACAAAT GGAGATACTC TCCTCGGTGG AGACGACTTT |
| | GATGAAGTCA |
| 701 | TTATCAAATG GATGATCGAA GAATTCAAAA AACAAGAAGG |
| | CATTGATCTT |
| 751 | AGCAAAGATA ATATGGCCTT ACAAAGACTT AAAGATGCTG |
| | CTGAGAAAGC |
| 801 | AAAAATAGAA CTTTCAGGAG TCTCTTCCAC AGAAATCAAT |
| | CAGCCATTCA |
| 851 | TCACAATGGA TGCACAAGGA CCTAAACACC TTGCATTGAC |
| | ACTCACACGT |
| 901 | GCGCAATTCG AGAAACTCGC AGCCTCTCTA ATCGAAAGAA |
| | CAAAATCTCC |
| 951 | ATGCATCAAA GCACTCAGTG ACGCAAAACT TTCCGCTAAG |
| | GATATCGATG |
| 1001 | ATGTTCTCTT AGTTGGAGGT ATGTCAAGAA TGCCCGCAGT |
| | GCAAGAAACT |
| 1051 | GTAAAAGAAC TCTTCGGCAA AGAGCCTAAT AAAGGAGTCA |
| | ACCCCGACGA |
| 1101 | AGTTGTTGCT ATTGGAGCCG CAATTCAAGG TGGTGTTCTT |
| | GGCGGAGAAG |
| 1151 | TTAAGGATGT TCTACTCCTA GACGTTATCC CCCTATCTCT |
| | GGGTATCGAA |
| 1201 | ACTCTAGGAG GCGTCATGAC GACTCTGGTA GAGAGAAATA |
| | CTACAATCCC |
| 1251 | TACACAGAAA AAACAAATCT TCTCCACAGC TGCTGATAAC |
| | CAGCCTGCGG |
| 1301 | TTACCATCGT AGTTCTCCAA GGAGAGCGTC CCATGGCCAA |
| | AGATAACAAG |
| 1351 | GAAATCGGAA GATTCGATCT TACAGATATC CCTCCGGCTC |
| | CTCGAGGCCA |
| 1401 | TCCTCAAATC GAAGTCTCCT TCGATATCGA TGCAAACGGA |
| | ATTTTCCATG |
| 1451 | TCTCAGCTAA AGATGTTGCC AGCGGTAAAG AACAGAAAAT |
| | TCGTATCGAA |
| 1501 | GCAAGCTCAG GACTTCAAGA AGATGAAATC CAAAGAATGG |
| | TTCGAGATGC |
| 1551 | CGAAATTAAT AAGGAAGAAG ATAAAAAACG TCGTGAAGCT |
| | TCAGATGCTA |
| 1601 | AAAATGAAGC CGATAGCATG ATCTTCAGAG CCGAAAAAGC |
| | TATTAAAGAT |
| 1651 | TATAAGGAGC AAATTCCTGA AACTTTAGTT AAAGAAATCG |
| | AAGAGCGAAT |
| 1701 | CGAAAACGTG CGCAACGCAC TCAAAGATGA CGCTCCTATT |
| | GAAAAAATTA |
| 1751 | AAGAGGTTAC TGAAGACCTA AGCAAGCATA TGCAAAAAAT |
| | TGGAGAGTCT |
| 1801 | ATGCAATCGC AGTCTGCATC AGCAGCAGCA TCATCGGCAG |
| | CCAATGCTAA |
| 1851 | AGGTGGACCT AACATCAATA CAGAAGATTT GAAAAAACAT |
| | AGTTTCAGTA |
| 1901 | CGAAGCCTCC TTCAAATAAC GGTTCTTCAG AAGACCATAT |
| | CGAAGAAGCT |
| 1951 | GATGTAGAAA TTATTGATAA CGACGATAAG TAA |

The PSORT algorithm predicts an inner membrane location (0.151).

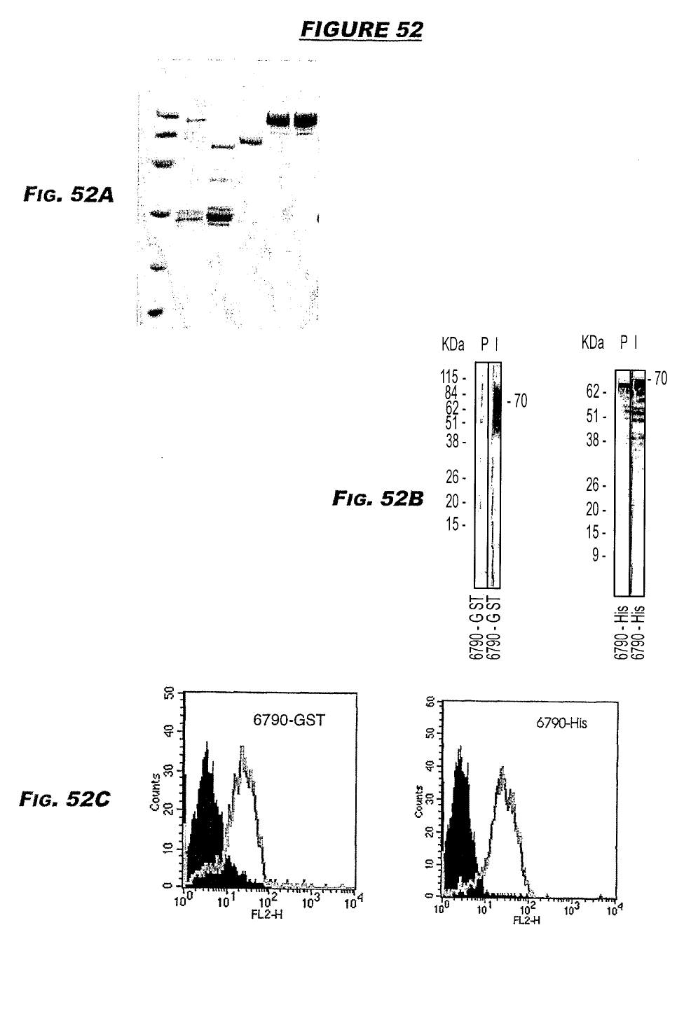

The protein was expressed in E. coli and purified as a GST-fusion product (FIG. 52A) and a his-tagged product. The proteins were used to immunise mice, whose sera were used in Western blot (FIG. 52B) and FACS (FIG. 52C) analyses.

The cp6790 protein was also identified in the 2D-PAGE experiment (Cpn0503).

These experiments show that cp6790 is a surface-exposed and immunoaccessible protein, and that it is a useful immunogen. These properties are not evident from the sequence alone.

### Example 53

The following C. pneumoniae protein (PID 4376878) was expressed <SEQ ID 105; cp6878>:

| | |
|---|---|
| 1 | MNVPDSKNLH PPAYELLEIK ARITQSYKEA SAILTAIPDG |
| | ILLLSFTGHF |
| 51 | LICNSQAREI LGIDENLEII NRSFTDVLPD TCLGFSIQEA |
| | LESLKVPKTL |
| 101 | RLSLCKESKE KEVELFIRKN EISGYLFIQI RDRSDYKQLE |
| | NAIERYKNIA |
| 151 | ELGKMTATLA HEIRNPLSGI VGFASILKKE ISSPRHQRML |
| | SSIISGTRSL |
| 201 | NNLVSSMLFY TKSQPLNLKI INLQDFFSSL IPLLSVSFPN |
| | CKFVREGAQP |
| 251 | LFRSIDPDRM NSVVWNLVKN AVETGNSPIT LTLHTSGDIS |
| | VTNPGTIPSE |
| 301 | IMDKLFTPFF TTKREGNGLG LAEAQKIIRL HGGDIQLKTS |
| | DSAVSFFIII |
| 351 | PELLAALPKE RAAS* |

The cp6878 nucleotide sequence <SEQ ID 106> is:

| | |
|---|---|
| 1 | ATGAACGTCC CTGATTCCAA GAACCTCCAT CCTCCTGCAT |
| | ACGAACTCCT |
| 51 | AGAGATCAAG GCTCGCATCA CACAATCTTA TAAAGAAGCG |
| | AGTGCTATAC |
| 101 | TGACAGCGAT TCCTGATGGT ATCCTATTAC TTTCTGAAAC |
| | AGGACACTTT |
| 151 | CTTATCTGCA ATTCACAAGC ACGTGAAATT CTAGGAATTG |
| | ATGAAAATCT |
| 201 | AGAAATTCTT AATAGATCCT TTACCGATGT TCTCCCCGAT |
| | ACGTGTCTTG |
| 251 | GATTTTCTAT TCAAGAGGCT CTTGAATCTC TAAAAGTCCC |
| | TAAAACTCTT |
| 301 | AGACTCTCTC TCTGTAAAGA ATCTAAAGAA AAAGAAGTGG |
| | AACTCTTCAT |
| 351 | CCGTAAAAAC GAGATCAGTG GATACCTGTT TATCCAAATC |
| | CGCGATCGGT |
| 401 | CCGACTATAA ACAACTAGAA AACGCTATAG AAAGATATAA |
| | AAATATCGCA |
| 451 | GAACTTGGGA AAATGACGGC TACCCTAGCT CACGAAATCC |
| | GCAATCCGCT |
| 501 | AAGTGGAATC GTTGGATTTG CCTCTATCCT AAAGAAAGAG |
| | ATTTCCTCTC |
| 551 | CTCGCCACCA ACGAATGCTC TCCTCAATCA TCTCCGGCAC |
| | AAGGTCTCTA |
| 601 | AATAACCTTG TCTCTTCTAT GTTAGAATAT ACAAAATCAC |
| | AACCGTTGAA |
| 651 | CCTAAAGATT ATAAATTTAC AAGACTTCTT CTCTTCTCTT |
| | ATCCCTCTGC |
| 701 | TCTCCGTCTC TTTCCCGAAT TGCAAGTTTG TAAGAGAGGG |
| | CGCACAACCT |
| 751 | CTATTCAGAT CTATAGATCC TGATCGGATG AACAGTGTCG |
| | TTTGGAACCT |
| 801 | AGTGAAAAAT GCTGTAGAAA CAGGGAACTC TCCGATCACT |
| | CTGACCCTGC |
| 851 | ATACATCGGG AGACATCTCG GTAACGAACC CCGGAACGAT |
| | TCCTTCCGAG |
| 901 | ATCATGGACA AGCTCTTCAC TCCATTCTTC ACAACAAAGA |
| | GAGAGGGAAA |
| 951 | TGGTTTGGGA CTTGCTGAAG CTCAAAAAAT TATAAGACTC |
| | CATGGAGGAG |
| 1001 | ATATCCAATT AAAAACAAGC GACTCCGCCG TTAGCTTCTT |
| | CATAATCATC |
| 1051 | CCCGAACTTC TAGCGGCCCT ACCCAAAGAA AGAGCCGCTA |
| | G |

The PSORT algorithm predicts an inner membrane location (0.204).

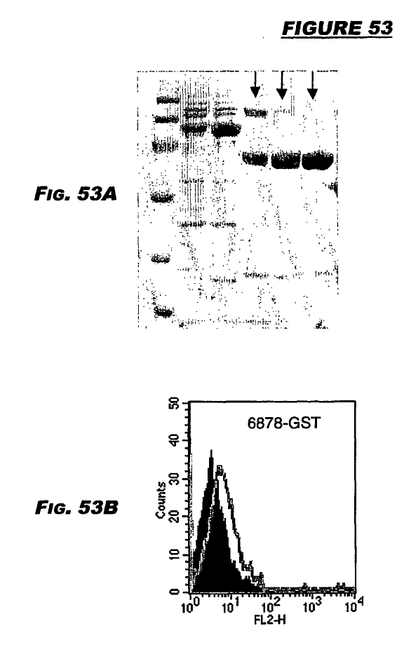

The protein was expressed in E. coli and purified as a his-tag product (FIG. 53A) and as a GST-fusion product. The recombinant GST-fusion protein was used to immunise mice, whose sera were used in a Western blot (FIG. 53B) and for FACS analysis.

These experiments show that cp6878 is a surface-exposed and immunoaccessible protein, and that it is a useful immunogen. These properties are not evident from the sequence alone.

### Example 54

The following C. pneumoniae protein (PID 4377224) was expressed <SEQ ID 107; cp7224>:

| | |
|---|---|
| 1 | MMKKIRKVAL AVGGSGGHIV PALSVKEAFS REGIDVLLLG |
| | KGLKNHPSLQ |
| 51 | QGISYREIPS GLPTVLNPIK IMSRTLSLCS GYLKARKELK |
| | IFDPDLVIGF |
| 101 | GSYHSLPVLL AGLSHKIPLF LHEQNLVPGK VNQLFSRYAR |
| | GIGVNFSPVT |
| 151 | KHFRCPAEEV FLPKRSFSLG SPMMKRCTNH TPTICVVGGS |
| | QGAQILNTCV |
| 201 | PQALVKLVNK YPNMYVHHIV GPKSDVMKVQ HVYNRGEVLC |
| | CVKPFEEQLL |
| 251 | DVLLAADIVI SRAGATILEE ILWAKVPGIL IPYPGAYGHQ |
| | EVNAKFFVDV |
| 301 | LEGGTMILEK ELTEKLLVEK VTFALDSHNR EKQRNSLAAY |
| | SQQRSTKTFH |
| 351 | AFICECL |

The cp7224 nucleotide sequence <SEQ ID 108> is:

| | |
|---|---|
| 1 | ATGATGAAGA AAATTCGAAA AGTAGCCTTG GCTGTAGGAG |
| | GTTCAGGAGG |
| 51 | CCACATTGTC CCAGCTCTCT CGGTAAAGGA AGCTTTTTCT |
| | CGTGAAGGAA |
| 101 | TAGACGTATT ACTACTAGGG AAAGGTCTCA AGAACCATCC |
| | TTCTTTGCAA |
| 151 | CAGGGAATCA GCTATCGGGA AATCCCCTCA GGACTTCCTA |
| | CAGTCCTTAA |
| 201 | TCCCATAAAG ATCATGAGCA GGACCCTTTC TCTATGTTCA |
| | GGATACCTGA |
| 251 | AAGCAAGAAA GGAACTTAAA ATTTTTGACC CTGACCTGGT |
| | CATAGGATTT |
| 301 | GGGAGCTACC ACTCTCTTCC CGTGTTGCTC GCAGGACTGT |
| | CCCATAAAAT |
| 351 | TCCCTTATTT CTACACGAAC AAAATCTAGT TCCTGGAAAA |
| | GTAAATCAAT |
| 401 | TGTTTTCCCG CTATGCTCGA GGTATTGGAG TGAATTTCTC |
| | CCCCGTTACT |
| 451 | AAACACTTCC GCTGCCCCGC AGAAGAGGTC TTCCTTCCTA |
| | AACGAAGCTT |
| 501 | CTCCTTAGGA AGCCCTATGA TGAAGCGATG TACAAATCAT |
| | ACCCCTACAA |
| 551 | TCTGTGTTGT TGGAGGTTCT CAGGGAGCAC AGATATTAAA |
| | TACTTGTGTT |
| 601 | CCCCAAGCTC TTGTCAAGCT AGTCAATAAG TACCCAAATA |
| | TGTACGTCCA |
| 651 | TCATATTGTA GGACCTAAAA GTGATGTTAT GAAGGTGCAA |
| | CATGTTTACA |
| 701 | ATCGTGGAGA GGTCCTCTGC TGTGTGAAGC CCTTCGAAGA |
| | GCAACTCCTA |
| 751 | GATGTCTTGC TTGCCGCAGA TTTGGTCATC AGTAGGGCAG |
| | GAGCCACAAT |
| 801 | TTTAGAAGAA ATTCTTTGGG CAAAAGTTCC CGGAATTTTA |
| | ATTCCCTATC |
| 851 | CAGGAGCTTA TGGACATCAG GAAGTTAATG CTAAATTCTT |
| | TGTAGACGTC |
| 901 | TTAGAAGGGG GAACTATGAT CCTAGAAAAA GAATTAACAG |
| | AGAAGCTATT |
| 951 | AGTAGAAAAA GTAACGTTTG CTTTAGACTC CCATAACAGA |
| | GAAAAACAAC |
| 1001 | GCAATTCCCT AGCGGCGTAT AGTCAGCAAA GGTCAACAAA |
| | AACATTCCAT |
| 1051 | GCATTCATTT GTGAATGCTT ATAG |

The PSORT algorithm predicts an inner membrane location (0.164).

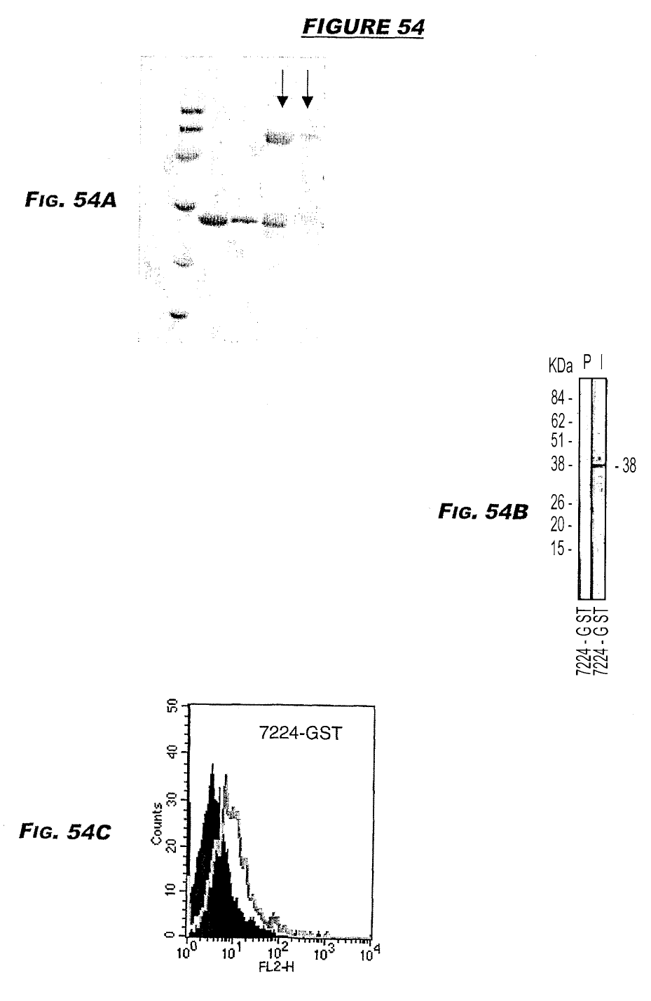

The protein was expressed in E. coli and purified as a GST-fusion product, as shown in FIG. 54A. The recombinant protein was used to immunise mice, whose sera were used in a Western blot (FIG. 54B) and for FACS analysis (FIG. 54C). A his-tagged protein was also expressed.

This protein also showed good cross-reactivity with human sera, including sera from patients with pneumonitis.

These experiments show that cp7224 is a surface-exposed and immunoaccessible protein, and that it is a useful immunogen. These properties are not evident from the sequence alone.

### Example 55

The following C. pneumoniae protein (PID 4377140) was expressed <SEQ ID 109; cp7140>:

| | |
|---|---|
| 1 | MVRRSISFCL FFLMTLLCCT SCNSRSLIVH GLPGREANEI |
| | VVLLVSKGVA |
| 51 | AQKLPQAAAA TAGAATEQMW DIAVRSAQIT EALAILNQAG |
| | LPRMKGTSLL |
| 101 | DLFAKQGLVP SELQEKIRYQ EGLSEQMAST IRKMDGVVDA |
| | SVQISFTTEN |
| 151 | EDNLRLTASV YIKHRGVLDN PNSIMVSKIK RLIASAVPGL |
| | VPENVSVVSD |
| 201 | RAAYSDITIN GPWGLTEEID YVSVWGIILA KSSLTKFRLI |
| | FYVLILILFV |
| 251 | ISCGLLWVIW KTHTLIMTMG GTKGFFNPTP YTKNALEAKK |
| | AEGAAADKEK |
| 301 | KEDADEQGES KNAETSDKDS SDKDAPEGSN EIEGA* |

A predicted signal peptide is highlighted.

The cp7140 nucleotide sequence <SEQ ID 110> is:

| | |
|---|---|
| 1 | ATGGTTCGTC GATCTATTTC TTTTTGCTTG TTCTTTCTAA |
| | TGACATTGCT |
| 51 | GTGCTGTACA AGCTGTAACA GCAGGTCTCT AATTGTGCAC |
| | GGTCTTCCTG |
| 101 | GCAGAGAAGC GAATGAGATT GTGGTGCTTT TGGTAAGCAA |
| | AGGGGTGGCT |
| 151 | GCACAAAAAT TGCCTCAAGC TGCAGCGGCT ACAGCCGGAG |
| | CAGCTACTGA |
| 201 | GCAAATGTGG GATATCGCGG TTCCGTCAGC ACAAATCACA |
| | GAGGCCCTTG |
| 251 | CCATTCTAAA TCAAGCGGGT CTTCCACGTA TGAAAGGGAC |
| | AAGCCTGTTA |
| 301 | GATCTTTTTG CAAAACAAGG TCTTGTTCCT TCCGAGCTTC |
| | AGGAAAAAAT |
| 351 | CCGTTATCAA GAAGGCTTAT CAGAACAGAT GGCCTCTACG |
| | ATTAGAAAAA |
| 401 | TGGATGGCGT TGTCGATGCC TCAGTACAGA TTTCCTTCAC |
| | TACAGAAAAT |
| 451 | GAAGATAATC TTCCTTTAAC AGCCTCTGTG TATATTAAGC |
| | ATCGAGGGGT |
| 501 | TTTGGACAAT CCGAACAGCA TTATGGTTTC CAAAATTAAG |
| | CGCCTTATTG |
| 551 | CAAGTGCTGT TCCAGGACTT GTGCCAGAGA ACGTCTCTGT |
| | AGTGAGCGAT |
| 601 | CGCGCAGCTT ATAGTGATAT TACAATTAAT GGTCCTTGGG |
| | GATTAACAGA |
| 651 | AGAAATCGAT TATGTTTCTG TTTGGGGTAT TATTCTTGCG |
| | AAGTCTTCGC |
| 701 | TCACCAAATT CCGTCTCATT TTTTATGTCT TGATTCTCAT |
| | TTTATTTGTT |
| 751 | ATTTCTTGTG GTCTCCTTTG GGTCATTTGG AAAACTCATA |
| | CTCTCATTAT |
| 801 | GACTATGGGA GGTACAAAAG GGTTCTTCAA CCCTACACCA |
| | TATACAAAGA |
| 851 | ATGCCTTGGA AGCCAAGAAA GCCGAGGGAG CAGCTGCTGA |
| | CAAAGAGAAA |
| 901 | AAAGAAGATG CAGATTCACA GGGGGAAAGC AAAAATGCGG |
| | AAACCAGTGA |
| 951 | TAAAGACTCT AGTGATAAAG ATGCTCCAGA AGGAAGCAAT |
| | GAAATTGAGG |
| 1001 | GTGCTTAG |

The PSORT algorithm predicts an inner membrane location (0.650).

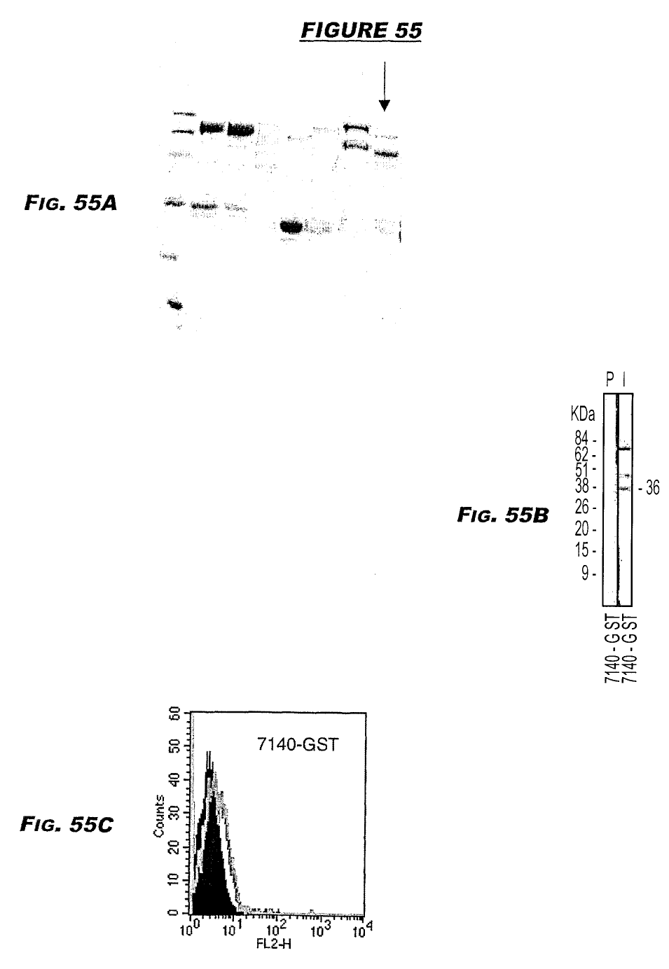

The protein was expressed in E. coli and purified as a GST-fusion product, as shown in FIG. 55A. The recombinant protein was used to immunise mice, whose sera were used in a Western blot (FIG. 55B) and for FACS analysis (FIG. 55C). A his-tagged protein was also expressed.

These experiments show that cp7140 is a surface-exposed and immunoaccessible protein, and that it is a useful immunogen. These properties are not evident from the sequence alone.

### Example 56

The following C. pneumoniae protein (PID 4377306) was expressed <SEQ ID 111; cp7306>:

| | |
|---|---|
| 1 | MITKQLRSWL AVLVGSSLLA LPLSGQAVGK KESRVSELPQ |
| | DVLLKEISGG |
| 51 | FSKVATKATP AVVYIESFPK SQAVTHPSPG RRGPYENPFD |
| | YFNDEFFNRF |
| 101 | FGLPSQREKP QSKEAVRGTG FLVSPDGYIV TNNHVVEDTG |
| | KIHVTLHDGQ |
| 151 | KYPATVIGLD PKTDLAVIKI KSQNLPYLSF GNSDHLKVGD |
| | WAIAIGNPFG |
| 201 | LQAIVIVGVI SAKGRNQLHI ADFEDFIQTD AAINPGNSGG |
| | PLLNIDGQVI |
| 251 | GVNTAIVSGS GGYIGIGFAI PSLMANRIID QLIRDGQVTR |
| | GFLGVTLQPI |
| 301 | DAELAACYKL ENVYGALVTD VVKGSPADKA GLEQEDVIIA |
| | YNGKEVDSLS |
| 351 | MFRNAVSLMN PDTRIVLKVV REGKVIEIPV TVSQAPKEDG |
| | MSALQRVGIR |
| 401 | VQNLTPETAK KLGIAPETKG ILIISVEPGS VAASSGIAPG |
| | QLILAVNRQK |
| 451 | VSSIEDLNRT IKDSNNENIL LMVSQGDVIR FIALKPEE* |

A predicted signal peptide is highlighted.

The cp7306 nucleotide sequence <SEQ ID 112> is:

| | |
|---|---|
| 1 | ATGATAACTA AGCAATTGCG TTCGTGGCTA GCTGTACTTG |
| | TTGGTTCAAG |
| 51 | TCTGCTAGCT CTTCCTTTAT CAGGGCAAGC TGTCGGGAAA |
| | AAAGAATCTC |
| 101 | GAGTTTCCGA GCTGCCTCAA GACGTTCTTC TTAAAGAGAT |
| | CTCGGGAGGG |
| 151 | TTTTCTAAGG TCGCTACCAA GGCGACTCCC GCTGTTGTGT |
| | ACATAGAAAG |
| 201 | TTTCCCAAAG AGCCAGGCTG TAACACATCC TTCTCCTGGA |
| | CGCCGTGGGC |
| 251 | CTTATGAAAA TCCTTTTGAT TATTTTAATG ATGAGTTTTT |
| | CAATCGTTTT |
| 301 | TTTGGTCTAC CTTCACAGAG GGAAAAACCT CAAAGTAAAG |
| | AGGCGGTTCG |
| 351 | AGGAACAGGT TTCCTAGTAT CTCCAGATGG CTATATTGTG |
| | ACTAATAACC |
| 401 | ATGTTGTCGA AGATACAGGT AAGATTCACG TAACTCTTCA |
| | TGATGGGCAA |
| 451 | AAGTACCCAG CAACTGTAAT CGGACTCGAT CCTAAAACAG |
| | ACCTTGCAGT |
| 501 | CATTAAAATT AAATCCCAAA ACCTCCCGTA TCTTTCTTTT |
| | GGAAACTCCG |
| 551 | ACCACTTAAA AGTCGGAGAT TGGGCAATTG CAATTGGAAA |
| | TCCCTTCGGT |
| 601 | CTTCAAGCTA CGGTCACCGT AGGTGTCATC AGTGCTAAAG |
| | GAAGAAATCA |
| 651 | ACTCCACATT GCAGATTTTG AAGATTTTAT TCAGACAGAT |
| | GCTGCGATTA |
| 701 | ATCCAGGCAA CTCTGGAGGC CCTCTTCTAA ATATTGATGG |
| | ACAGGTCATC |
| 751 | GGTGTTAATA CTGCCATTGT CAGTGGTAGT GGTGGCTATA |
| | TTGGAATCGG |
| 801 | GTTTGCGATT CCTAGCCTTA TGGCAAATAG AATCATAGAT |
| | CAGCTGATTC |
| 851 | GTGATGGTCA AGTTACCCGA GGATTCTTAG GAGTGACTTT |
| | ACAACCTATA |
| 901 | GATGCGGAAC TCGCTGCTTG CTACAAACTC GAAAAGGTTT |
| | ATGGCGCTTT |
| 951 | AGTCACAGAT GTTGTTAAAG GATCTCCAGC AGATAAAGCA |
| | GGGCTAAAAC |
| 1001 | AAGAAGATGT GATCATTGCT TATAATGGGA AAGAAGTCGA |
| | TTCACTGAGT |
| 1051 | ATGTTCCGTA ATGCTGTTTC TTTAATGAAT CCAGATACAC |
| | GTATTGTTCT |
| 1101 | AAAGGTAGTT CGTGAAGGAA AGGTTATCGA AATACCCGTG |
| | ACAGTTTCTC |
| 1151 | AAGCTCCAAA AGAAGATGGA ATGTCGGCTT TACAGCGTGT |
| | GGGAATCCGT |
| 1201 | GTGCAAAACC TAACTCCTGA AACTGCTAAG AAGCTGGGAA |
| | TTGCTCCAGA |
| 1251 | GACTAAAGGC ATTTTGATTA TAAGTGTTGA ACCAGGGTCT |
| | GTAGCAGCTT |
| 1301 | CTTCAGGAAT TGCTCCTGGT CAGCTGATCC TTGCTGTGAA |
| | TAGACAAAAA |
| 1351 | GTATCTTCGA TTGAAGATCT GAATAGAACG TTAAAAGATT |
| | CTAACAATGA |
| 1401 | GAATATTCTT CTTATGGTTT CTCAAGGAGA TGTTATTCGC |
| | TTCATTGCCC |
| 1451 | TGAAACCTGA AGAATAA |

The PSORT algorithm predicts a periplasmic location (0.923).

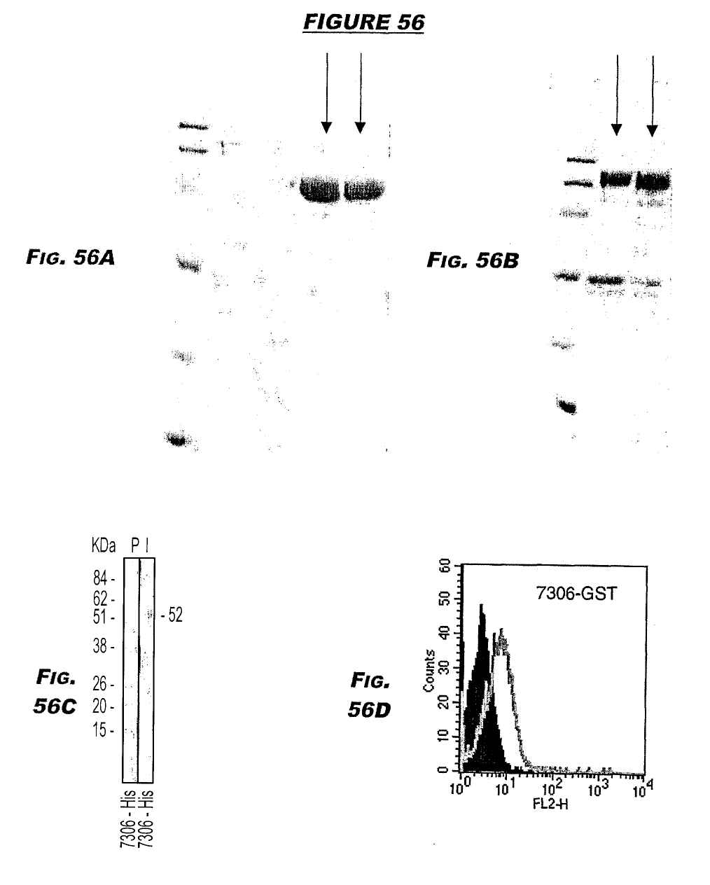

The protein was expressed in E. coli and purified as a his-tag product (FIG. 56A) and as a GST-fusion product (FIG. 56B). The recombinant proteins were used to immunise mice, whose sera were used in a Western blot (FIG. 56C) and for FACS (FIG. 56D) analyses.

The cp7306 protein was also identified in the 2D-PAGE experiment (Cpn0979) and showed good cross-reactivity with human sera, including sera from patients with pneumonitis.

These experiments show that cp7306 is a surface-exposed and immunoaccessible protein, and that it is a useful immunogen. These properties are not evident from the sequence alone.

### Example 57

The following C. pneumoniae protein (PID 4377132) was expressed <SEQ ID 113; cp7132>:

| | |
|---|---|
| 1 | MCNSIAMKKQ KRGFVLMELL MSFTLIALLL GTLGFWYRKI |
| | YTVQKQKERI |
| 51 | YNFYIEESRA YKQLRTLFSM SLSSSYEEPG SLFSLIFDRG |
| | VYRDPKLAGA |
| 101 | VRASLHHDTK DQRLELRICN IKDQSYFETQ RLLSHVTHVV |
| | LSFQRNPDPE |
| 151 | KLPETIALTI TREPKAYPPR TLTYQFAVGK* |

A predicted signal peptide is highlighted.

The cp7132 nucleotide sequence <SEQ ID 114> is:

| | |
|---|---|
| 1 | ATGTGTAACT CTATAGCTAT GAAAAAGCAA AAGCGTGGCT |
| | TTGTGCTTAT |
| 51 | GGAATTACTC ATGTCGTTCA CTCTAATTGC TTTGTTATTA |
| | GGGACTTTAG |
| 101 | GATTTTGGTA TCGGAAAATT TATACTGTAC AAAAGCAAAA |
| | AGAACGTATT |
| 151 | TATAACTTTT ATATCGAAGA AAGCCGAGCC TACAAGCAGC |
| | TCAGAACCCT |
| 201 | GTTTAGCATG TCCTTGTCTT CATCTTACGA GGAGCCTGGA |
| | TCATTATTTT |
| 251 | CTTTAATCTT TGATCGGGGT GTTTATCGAG ATCCTAAGCT |
| | GGCAGGTGCG |
| 301 | GTACGAGCTT CTCTCCATCA TGACACCAAG GATCAGAGAT |
| | TGGAACTTCG |
| 351 | TATTTGTAAT ATTAAGGATC AGTCTTACTT TGAAACACAG |
| | CGACTGCTCT |
| 401 | CCCACGTGAC CCATGTTGTA CTTTCCTTCC AGAGAAATCC |
| | TGATCCTGAA |
| 451 | AAACTTCCTG AAACAATTGC TTTAACTATA ACACGGGAAC |
| | CTAAAGCATA |
| 501 | TCCTCCAAGG ACGTTAACAT ACCAATTTGC GGTTGGGAAA |
| | TAA |

The PSORT algorithm predicts a periplasmic location (0.915).

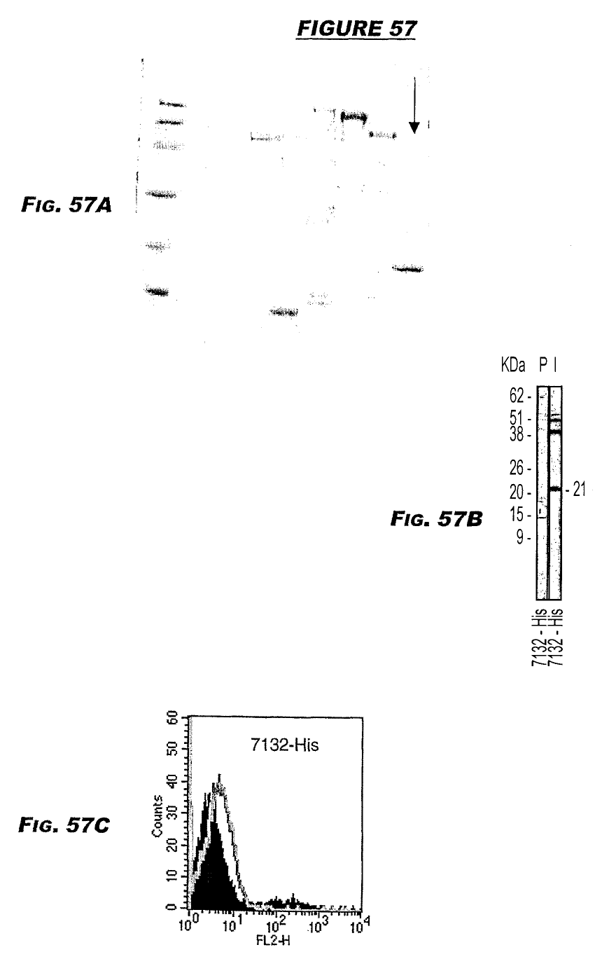

The protein was expressed in E. coli and purified as a his-tag product (FIG. 57A) or as a GST-fusion. The recombinant proteins were used to immunise mice, whose sera were used in a Western blot (FIG. 57B) and FACS (FIG. 57C) analyses.

These experiments show that cp7132 is a surface-exposed and immunoaccessible protein, and that it is a useful immunogen. These properties are not evident from the sequence alone.

### Example 58

The following C. pneumoniae protein (PID 4376733) was expressed <SEQ ID 115; cp6733>:

| | |
|---|---|
| 1 | MKTSIPWVLV SSVLAFSCHL QSLANEELLS PDDSFNGNID |
| | SGTFTPKTSA |
| 51 | TTYSLTGDVF FYEPGKGTPL SDSCFKQTTD NLTFLGNGHS |
| | LTFGFIDAGT |
| 101 | HAGAAASTTA NKNLTFSGFS LLSFDSSPST TVTTGQGTLS |
| | SAGGVNLENI |
| 151 | RKLVVAGNFS TADGGAIKGA SFLLTGTSGD ALFSNNSSST |
| | KGGAIATTAG |
| 201 | ARIANNTGYV RFLSNIASTS GGAIDDEGTS ILSNNKFLYF |
| | EGNAAKTTGG |
| 251 | AICNTKASGS PELIISNNKT LIFASNVAET SGGAIHAKKL |
| | ALSSGGFTEF |
| 301 | LRNNVSSATP KGGAISIDAS GELSLSAETG NITFVRNTLT |
| | TTGTDTPRKR |
| 351 | NAINIGSNGK FTELRAAKNH TIFFYDPITS EGTSSDVLKI |
| | NNGSAGALNP |
| 401 | YQGTILFSGE TLTADELKVA DNLKSSFTQP VSLSGGKLLL |
| | QKGVTLESTS |
| 451 | FSQEAGSLLG MDSGTTLSTT AGSITITNLG INVDSLGLKQ |
| | PVSLTAKGAS |
| 501 | NKVIVSGKLN IIDIEGNIYE SHMFSHDQLF SLLKITVDAD |
| | VDTNVDISSL |
| 551 | IPVPAEDPNS EYGFQGQWNV MWTTDTATNT KEATATWTKT |
| | GFVPSPERKS |
| 601 | ALVCNTLWGV FTDIRSLQQL VEIGATGMEH KWGFWVSSMT |
| | NFLHKTGDEN |
| 651 | RKGFRHTSGG YVIGGSAHTP KDDLFTFAFC HLFARDKDCF |
| | IAHNNSRTYG |
| 701 | GTLFFKHSHT LQPQNYLRLG RAKFSESAIE KFPREIPLAL |
| | DVQVSFSHSD |
| 751 | NRMETHYTSL PESEGSWSNE CIAGGIGLDL PFVLSNPHPL |
| | FKTFIPQMKV |
| 801 | EMVYVSQNSF FESSSDGRGF SIGRLLNLSI PVGAKFVQGD |
| | IGDSYTYDLS |
| 851 | GFFVSDVYRN NPQSTATLVM SPDSWKIRGG NLSRQAFLLR |
| | GSNNYVYNSN |
| 901 | CELFGHYAME LRGSSRNYNV DVGTKLRF* |

A predicted signal peptide is highlighted.

The cp6733 nucleotide sequence <SEQ ID 116> is:

| | |
|---|---|
| 1 | ATGAAGACTT CGATTCCTTG GGTTTTAGTT TCCTCCGTGT |
| | TAGCTTTCTC |
| 51 | ATGTCACCTA CAGTCACTAG CTAACGAGGA ACTTTTATCA |
| | CCTGATGATA |
| 101 | GCTTTAATGG AAATATCGAT TCAGGAACGT TTACTCCAAA |
| | AACTTCAGCC |
| 151 | ACAACATATT CTCTAACAGG AGATGTCTTC TTTTACGAGC |
| | CTGGAAAAGG |
| 201 | CACTCCCTTA TCTGACAGTT GTTTTAAGCA AACCACGGAC |
| | AATCTTACCT |
| 251 | TCTTGGGGAA CGGTCATAGC TTAACGTTTG GCTTTATAGA |
| | TGCTGGCACT |
| 301 | CATGCAGGTG CTGCTGCATC TACAACAGCA AATAAGAATC |
| | TTACCTTCTC |
| 351 | AGGGTTTTCC TTACTGAGTT TTGATTCCTC TCCTAGCACA |
| | ACGGTTACTA |
| 401 | CAGGTCAGGG AACGCTTTCC TCAGCAGGAG GCGTAAATTT |
| | AGAAAATATT |
| 451 | CGTAAACTTG TAGTTGCTGG GAATTTTTCT ACTGCAGATG |
| | GTGGAGCTAT |
| 501 | CAAAGGAGCG TCTTTCCTTT TAACTGGCAC TTCTGGAGAT |
| | GCTCTTTTTA |
| 551 | GTAACAACTC TTCATCAACA AAGGGAGGAG CAATTGCTAC |
| | TACAGCAGGC |
| 601 | GCTCGCATAG CAAATAACAC AGGTTATGTT AGATTCCTAT |
| | CTAACATAGC |
| 651 | GTCTACGTCA GGAGGCGCTA TCGATGATGA AGGCACGTCG |
| | ATACTATCGA |
| 701 | ACAACAAATT TCTATATTTT GAAGGGAATG CAGCGAAAAC |
| | TACTGGCGGT |
| 751 | GCGATCTGCA ACACCAAGGC GAGTGGATCT CCTGAACTGA |
| | TAATCTCTAA |
| 801 | CAATAAGACT CTGATCTTTG CTTCAAACGT AGCAGAAACA |
| | AGCGGTGGCG |
| 851 | CCATCCATGC TAAAAAGCTA GCCCTTTCCT CTGGAGGCTT |
| | TACAGAGTTT |
| 901 | CTACGAAATA ATGTCTCATC AGCAACTCCT AAGGGGGGTG |
| | CTATCAGCAT |
| 951 | CGATGCCTCA GGAGAGCTCA GTCTTTCTGC AGAGACAGGA |
| | AACATTACCT |
| 1001 | TTGTAAGAAA TACCCTTACA ACAACCGGAA GTACCGATAC |
| | TCCTAAACGT |
| 1051 | AATGCGATCA ACATAGGAAG TAACGGGAAA TTCACGGAAT |
| | TACGGGCTGC |
| 1101 | TAAAAATCAT ACAATTTTCT TCTATGATCC CATCACTTCA |
| | GAAGGAACCT |
| 1151 | CATCAGACGT ATTGAAGATA AATAACGGCT CTGCGGGAGC |
| | TCTCAATCCA |
| 1201 | TATCAAGGAA CGATTCTATT TTCTGGAGAA ACCCTAACAG |
| | CAGATGAACT |
| 1251 | TAAAGTTGCT GACAATTTAA AATCTTCATT CACGCAGCCA |
| | GTCTCCCTAT |
| 1301 | CCGGAGGAAA GTTATTGCTA CAAAAGGGAG TCACTTTAGA |
| | GAGCACGAGC |
| 1351 | TTCTCTCAAG AGGCCGGTTC TCTCCTCGGC ATGGATTCAG |
| | GAACGACATT |
| 1401 | ATCAACTACA GCTGGGAGTA TTACAATCAC GAACCTAGGA |
| | ATCAATGTTG |
| 1451 | ACTCCTTAGG TCTTAAGCAG CCCGTCAGCC TAACAGCAAA |
| | AGGTGCTTCA |
| 1501 | AATAAAGTGA TCGTATCTGG GAAGCTCAAC CTGATTGATA |
| | TTGAAGGGAA |
| 1551 | CATTTATGAA AGTCATATGT TCAGCCATGA CCAGCTCTTC |
| | TCTCTATTAA |
| 1601 | AAATCACGGT TGATGCTGAT GTTGATACTA ACGTTGACAT |
| | CAGCAGCCTT |
| 1651 | ATCCCTGTTC CTGCTGAGGA TCCTAATTCA GAATACGGAT |
| | TCCAAGGACA |
| 1701 | ATGGAATGTT AATTGGACTA CGGATACAGC TACAAATACA |
| | AAAGAGGCCA |
| 1751 | CGGCAACTTG GACCAAAACA GGATTTGTTC CCAGCCCCGA |
| | AAGAAAATCT |
| 1801 | GCGTTAGTAT GCAATACCCT ATGGGGAGTC TTTACTGACA |
| | TTCGCTCTCT |
| 1851 | GCAACAGCTT GTAGAGATCG GCGCAACTGG TATGGAACAC |
| | AAACAAGGTT |
| 1901 | TCTGGGTTTC CTCCATGACG AACTTCCTGC ATAAGACTGG |
| | AGATGAAAAT |
| 1951 | CGCAAAGGCT TCCGTCATAC CTCTGGAGGC TACGTCATCG |
| | GTGGAAGTGC |
| 2001 | TCACACTCCT AAAGACGACC TATTTACCTT TGCGTTCTGC |
| | CATCTCTTTG |
| 2051 | CTAGAGACAA AGATTGTTTT ATCGCTCACA ACAACTCTAG |
| | AACCTACGGT |
| 2101 | GGAACTTTAT TCTTCAAGCA CTCTCATACC CTACAACCCC |
| | AAAACTATTT |
| 2151 | GAGATTAGGA AGAGCAAAGT TTTCTGAATC AGCTATAGAA |
| | AAATTCCCTA |
| 2201 | GGGAAATTCC CCTAGCCTTG GATGTCCAAG TTTCGTTCAG |
| | CCATTCAGAC |
| 2251 | AACCGTATGG AAACGCACTA TACCTCATTG CCAGAATCCG |
| | AAGGTTCTTG |
| 2301 | GAGCAACGAG TGTATAGCTG GTGGTATCGG CCTAGACCTT |
| | CCTTTTGTTC |
| 2351 | TTTCCAACCC ACATCCTCTT TTCAAGACCT TCATTCCACA |
| | GATGAAAGTC |
| 2401 | GAAATGGTTT ATGTATCACA AAATAGCTTC TTCGAAAGCT |
| | CTAGTGATGG |
| 2451 | CCGTGGTTTT AGTATTGGAA GGCTGCTTAA CCTCTCGATT |
| | CCTGTGGGTG |
| 2501 | CGAAATTCGT GCAGGGGGAT ATCGGAGATT CCTACACCTA |
| | TGATCTCTCA |
| 2551 | GGATTCTTTG TTTCCGATGT CTATCGTAAC AATCCCCAAT |
| | CTACAGCGAC |
| 2601 | TCTTGTGATG AGCCCAGACT CTTGGAAAAT TCGCGGTGGC |
| | AATCTTTCAA |
| 2651 | GACAGGCATT TTTACTGAGG GGTAGCAACA ACTACGTCTA |
| | CAACTCCAAT |
| 2701 | TGTGAGCTCT TCGGACATTA CGCTATGGAA CTCCGTGGAT |
| | CTTCAAGGAA |
| 2751 | CTACAATGTA GATGTTGGTA CCAAACTCCG ATTCTAG |

The PSORT algorithm predicts an outer membrane location (0.924).

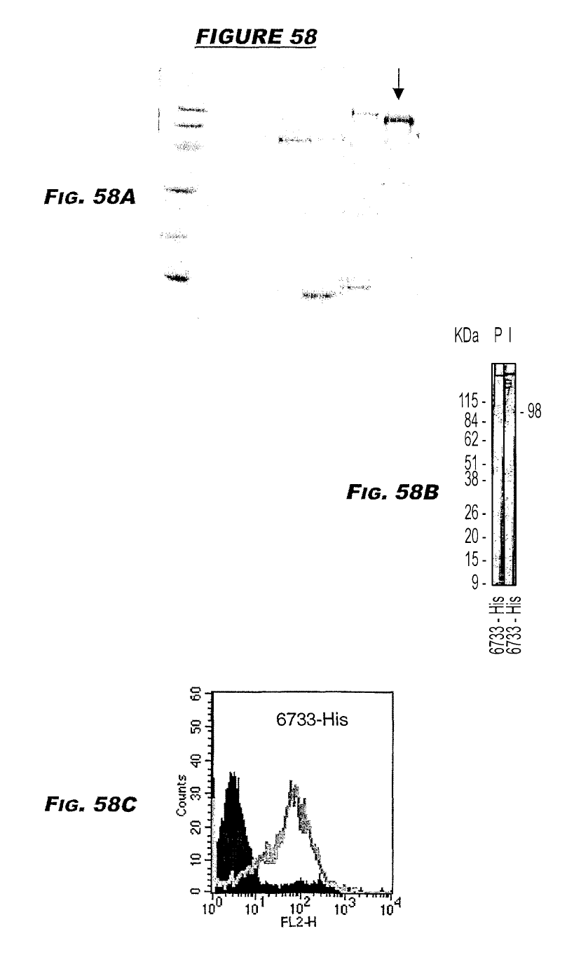

The protein was expressed in E. coli and purified as a his-tag product, as shown in FIG. 58A. The recombinant protein was used to immunise mice, whose sera were used in a Western blot (FIG. 58B) and for FACS (FIG. 58C) analyses. A GST-fusion protein was also expressed.

The cp6733 protein was also identified in the 2D-PAGE experiment (Cpn0451).

These experiments show that cp6733 is a surface-exposed and immunoaccessible protein, and that it is a useful immunogen. These properties are not evident from the sequence alone.

### Example 59

The following C. pneumoniae protein (PID 4376814) was expressed <SEQ ID 117; cp6814>:

| | |
|---|---|
| 1 | MHDALLSILA IQELDIKMIR LMRVKKEHQK ELAKVQSLKS |
| | DIRRKVQEKE |
| 51 | LEMENLKTQI RDGENRIQEI SEQINKLENQ QAAVKKMDEF |
| | NALTQEMTTA |
| 101 | NKERRSLEHQ LSDLMDKQAG GEDLIVSLKE SLASTENSSS |
| | VIEKEIFESI |
| 151 | KKINEEGKAL LEQRTELKHA TNPELLSIYE RLLNNKKDRV |
| | VVPIENRVCS |
| 201 | GCHIVLTPQH ENLVRKKDRL IFCEHCSRIL YWQESQVKAQ |
| | ENSTAKRRRR |
| 251 | RAAV* |

The cp6814 nucleotide sequence <SEQ ID 118> is:

| | |
|---|---|
| 1 | ATGCATGACG CACTTCTAAG CATTTTGGCT ATTCAAGAGC |
| | TTGATATTAA |
| 51 | AATGATTCGC CTTATGCGCG TAAAGAAAGA ACATCAGAAA |
| | GAATTGGCTA |
| 101 | AAGTCCAATC TTTAAAAAGT GATATTCGTA GAAAAGTTCA |
| | GGAAAAAGAA |
| 151 | CTCGAAATGG AGAATTTGAA AACTCAAATT CGAGATGGAG |
| | AGAATCGCAT |
| 201 | CCAAGAGATT TCTGAACAAA TCAATAAATT AGAAAATCAG |
| | CAAGCTGCTG |
| 251 | TAAAAAAAAT GGATGAGTTT AACGCTCTTA CCCAAGAAAT |
| | GACTACAGCA |
| 301 | AACAAAGAAC GTCGCTCTTT AGAGCACCAG CTTAGCGATC |
| | TCATGGATAA |
| 351 | GCAAGCTGGA GGCGAAGACC TTATTGTCTC TCTAAAAGAA |
| | AGCTTAGCTT |
| 401 | CTACAGAAAA TAGTAGCAGT GTCATTGAAA AAGAAATTTT |
| | TGAAAGCATC |
| 451 | AAAAAGATTA ATGAAGAAGG CAAAGCTTTG CTTGAACAAC |
| | GGACAGAGTT |
| 501 | AAAGCATGCG ACGAATCCCG AACTACTCAG CATCTATGAG |
| | CGTCTATTAA |
| 551 | ACAATAAAAA AGATCGCGTT GTTGTTCCTA TTGAAAATCG |
| | TGTCTGCAGT |
| 601 | GGTTGTCATA TTGTTCTAAC TCCTCAACAC GAAAATCTTG |
| | TAAGAAAGAA |
| 651 | AGACCGACTC ATTTTTTGCG AACATTGCTC TCGAATTCTC |
| | TATTGGCAAG |
| 701 | AATCCCAAGT CAATGCTCAG GAAAATTCCA CAGCAAAACG |
| | TCGTCGTCGT |
| 751 | CGCGCAGCTG TATAA |

The PSORT algorithm predicts an inner membrane location (0.070).

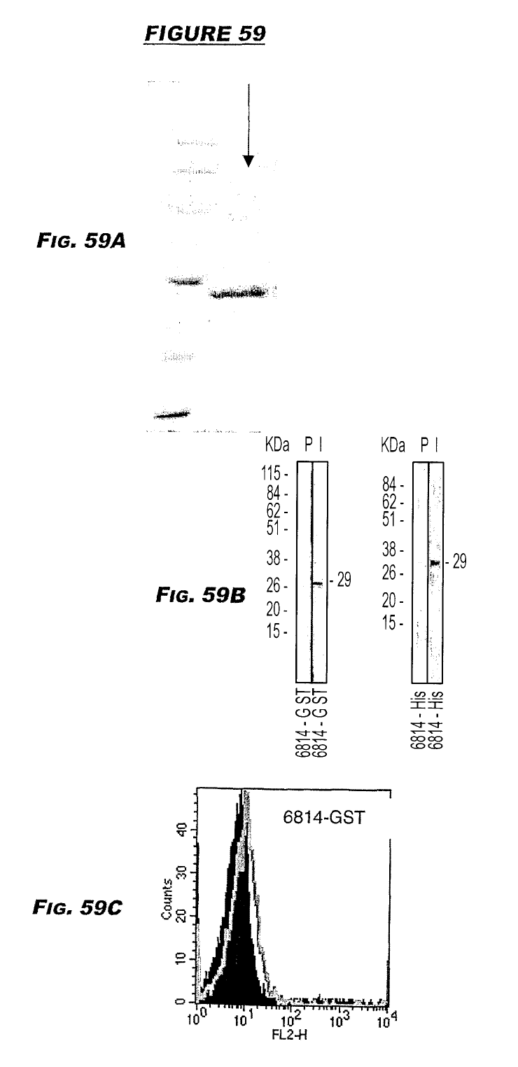

The protein was expressed in E. coli and purified as a GST-fusion (FIG. 59A) or his-tagged product. The recombinant proteins were used to immunise mice, whose sera were used in Western blot (FIG. 59B) and FACS (FIG. 59C) analyses.

These experiments show that cp6814 is a surface-exposed and immunoaccessible protein, and that it is a useful immunogen. These properties are not evident from the sequence alone.

### Example 60

The following C. pneumoniae protein (PID 4376830) was expressed <SEQ ID 119; cp6830>:

| | |
|---|---|
| 1 | MKWLPATAVF AAVLPALTAF GDPASVEIST SHTGSGDPTS |
| | DAALTGFTQS |
| 51 | STETDGTTYT IVGDITFSTF TNIPVPVVTP DANDSSSNSS |
| | KGGSSSSGAT |
| 101 | SLIRSSNLHS DFDFTKDSVL DLYHLFFPSA SNTLNPALLS |
| | SSSSGGSSSS |
| 151 | SSSSSSGSAS AVVAADPKGG AAFYSNEANG TLIFTTDSGN |
| | PGSLTLQNLK |
| 201 | MTGDGAAIYS KGPLVFTGLK NLTFTGNESQ KSGGAAYTEG |
| | ALTTQAIVEA |
| 251 | VTFTGNTSAG QGGAIYVKEA TLFNALDSLK FEKNTSGQAG |
| | GGIYTESTLT |
| 301 | ISNITKSIEF ISNKASVPAP APEPTSPAPS SLINSTTIDT |
| | STLQTRAASA |
| 351 | TPAVAPVAAV TPTPISTQET AGNGGAIYAK QGISISTFKD |
| | LTFKSNSASV |
| 401 | DATLTVDSST IGESGGAIFA ADSIQIGGCT GTTLFSGNTA |
| | NKSGGGIYAV |
| 451 | GQVTLEDIAN LKMTNNTCKG EGGAIYTKKA LTINNGAILT |
| | TFSGNTSTDN |
| 501 | GGAIFAVGGI TLSDLVEVRF SKNKTGNYSA PITKAASNTA |
| | PVVSSSTTAA |
| 551 | SPAVPAAAAA FVTNAAKGGA LYSTEGLTVS GITSILSFEN |
| | NECQNQGGGA |
| 601 | YVTKTFQCSD SHRLQFTSNK AADEGGGLYC GDDVTLTNLT |
| | GKTLFQENSS |
| 651 | EKHGGGLSLA SGKSLTMTSL ESFCLNANTA KENGGGANVP |
| | ENIVLTFTYT |
| 701 | PTPNEPAPVQ QPVYGEALVT GNTATKSGGG IYTKNAAFSN |
| | LSSVTFDQNT |
| 751 | SSENGGALLT QKAADKTDCS FTYITNVNIT NNTATGNGGG |
| | IAGGKAHFDR |
| 801 | IDNLTVQSNQ AKKGGGVYLE DALILEKVIT GSVSQNTATE |
| | SGGGIYAKDI |
| 851 | QLQALPGSFT ITDNKVETSL TTSTNLYGGG IYSSGAVTLT |
| | NISGTFGITG |
| 901 | NSVINTATSQ DADIQGGGIY ATTSLSINQC NTPILFSNNS |
| | AATKKTSTTK |
| 951 | QIAGGAIFSA AVTIENNSQP IIFLNNSAKS EATTAATAGN |
| | KDSCGGAIAA |
| 1001 | NSVTLTNNPE ITFKGNYAET GGAIGCIDDT NGSPPRKVSI |
| | ADNGSVLFQD |
| 1051 | NSALNRGGAI YGETIDISRT GATFIGNSSK HDGSAICCST |
| | ALTLAPNSQL |
| 1101 | IFENNKVTET TATTKASINN LGAAIYGNNE TSDVTISLSA |
| | ENGSIFFKNN |
| 1151 | LCTATNKYCS IAGNVKFTAI EASAGKAISF YDAVNVSTKE |
| | TNAQELKLNE |
| 1201 | KATSTGTILF SGELHENKSY IPQKVTFAHG NLILGKVAEL |
| | SVVSFTQSPG |
| 1251 | TTITMGPGSV LSNHSKEAGG IAINNVIIDF SEIVPTKDNA |
| | TVAPPTLKLV |
| 1301 | SRTNADSKDK IDITGTVTLL DPNGNLYQNS YLGEDRDITL |
| | FNIDNSASGA |
| 1351 | VTATNVTLQG NLGAKKGYLG TWNLDPNSSG SKIILKWTFD |
| | KYLRWPYIPR |
| 1401 | DNHFYINSIW GAWNSLVTVK QSILGNMLNN ARFEDPAFNN |
| | FWASAIGSFL |
| 1451 | RKEVSRNSDS FTYHGRGYTA AVDAKPRQEF ILGAAFSQVF |
| | GHAESEYHLD |
| 1501 | NYKHKGSGHS TQASIYAGNI FYFPAIRSRP ILFQGVATYG |
| | YMQHDTTTYY |
| 1551 | PSIEEKNMAN WDSIAWLFDL RFSVDLKEPQ PHSTARLTFY |
| | TEAEYTRIRQ |
| 1601 | EKFTELDYDP RSFSACSYGN LAIPTGFSVD GALAWREIIL |
| | YNKVSAAYLP |
| 1651 | VILRNNPKAT YEVLSTKEKG NVVNVLPTRN AARAEVSSQI |
| | YLGSYWTLYG |
| 1701 | TYTIDASMNT LVQMANGGIR FVF* |

A predicted signal peptide is highlighted.

The cp6830 nucleotide sequence <SEQ ID 120> is:

| | |
|---|---|
| 1 | ATGAAGTGGC TACCAGCTAC AGCTGTTTTT GCTGCCGTAC |
| | TCCCCGCACT |
| 51 | AACAGCCTTC GGAGATCCCG CGTCTGTTGA AATAAGTACC |
| | AGCCATACAG |
| 101 | GATCCGGGGA TCCTACAAGC GACGCTGCCT TAACAGGATT |
| | TACACAAAGT |
| 151 | TCCACAGAAA CTGACGGTAC TACCTATACC ATTGTCGGTG |
| | ATATCACCTT |
| 201 | CTCTACTTTT ACGAATATTC CTGTTCCCGT AGTAACTCCA |
| | GACGCCAACG |
| 251 | ATAGTTCCAG CAATAGCTCT AAAGGAGGAA GTAGCAGTAG |
| | TGGAGCTACA |
| 301 | TCTCTAATCC GATCCTCAAA CCTACACTCC GATTTTGATT |
| | TTACAAAAGA |
| 351 | TAGCGTGTTA GACCTCTATC ACCTTTTCTT TCCTTCAGCT |
| | TCAAATACTC |
| 401 | TCAATCCTGC ACTCCTTTCT TCCAGTAGCA GCGGTGGATC |
| | CTCGAGCAGC |
| 451 | AGTAGCTCCT CATCATCTGG AAGTGCATCT GCTGTTGTTG |
| | CTGCGGACCC |
| 501 | AAAAGGAGGC GCTGCCTTTT ATAGTAACGA GGCTAACGGA |
| | ACTTTAACCT |
| 551 | TCACTACAGA CTCTGGAAAT CCCGGCTCCC TGACTCTTCA |
| | GAATCTTAAA |
| 601 | ATGACCGGAG ATGGAGCCGC CATCTACTCG AAGGGTCCTC |
| | TAGTATTTAC |
| 651 | TGGTTTAAAA AATCTAACCT TTACAGGAAA TGAATCTCAG |
| | AAATCTGGAG |
| 701 | GTGCTGCCTA TACTGAAGGC GCACTCACAA CACAAGCAAT |
| | CGTTGAAGCC |
| 751 | GTAACTTTTA CTGGCAACAC CTCGGCAGGG CAAGGAGGCG |
| | CTATCTATGT |
| 801 | TAAAGAAGCT ACCCTATTCA ATGCTCTAGA CAGCCTCAAA |
| | TTTGAAAAAA |
| 851 | ACACTTCTGG GCAAGCTGGT GGTGGAATCT ATACAGAGTC |
| | TACGCTCACA |
| 901 | ATCTCGAACA TCACAAAATC TATTGAATTT ATCTCTAATA |
| | AAGCTTCTGT |
| 951 | CCCTGCCCCC GCTCCTGAGC CCACCTCTCC GGCTCCAAGT |
| | AGCTTAATAA |
| 1001 | ATTCTACAAC GATCGATACC TCGACTCTCC AAACCCGAGC |
| | AGCATCCGCA |
| 1051 | ACTCCAGCAG TGGCTCCTGT TGCTGCCGTA ACTCCAACAC |
| | CAATCTCTAC |
| 1101 | TCAAGAGACC GCAGGAAATG GAGGCGCTAT CTATGCTAAA |
| | CAAGGTATTT |
| 1151 | CGATATCCAC GTTTAAAGAT CTGACCTTCA AGTCTAACTC |
| | TGCATCGGTA |
| 1201 | GATGCCACCC TTACTGTCGA TTCTAGCACT ATTGGAGAAT |
| | CTGGAGGTGC |
| 1251 | TATCTTTGCA GCAGACTCTA TACAAATCCA ACAGTGCACG |
| | GGAACCACCT |
| 1301 | TATTCAGTGG CAATACTGCC AATAAGTCTG GTGGGGGTAT |
| | TTACGCTGTA |
| 1351 | GGACAAGTCA CCCTAGAAGA TATAGCGAAT CTGAAGATGA |
| | CCAACAACAC |
| 1401 | CTGTAAAGGT GAAGGTGGAG CCATCTACAC TAAAAAGGCT |
| | TTAACTATCA |
| 1451 | ACAACGGTGC CATTCTCACT ACATTTTCTG GAAATACATC |
| | GACAGATAAT |
| 1501 | GGTGGGGCTA TTTTTGCTGT AGGTGGCATC ACTCTCTCTG |
| | ATCTTGTAGA |
| 1551 | AGTCCGCTTT AGTAAAAATA AGACCGGAAA TTATTCCGCT |
| | CCTATTACCA |
| 1601 | AAGCGGCTAG CAACACAGCT CCTGTAGTTT CTAGCTCTAC |
| | AACTGCTGCA |
| 1651 | TCTCCTGCGG TCCCTGCTGC CGCTGCAGCA CCTGTTACAA |
| | ACGCAGCAAA |
| 1701 | AGGAGGGGCT TTATATAGTA CAGAAGGACT GACTGTATCT |
| | GGAATCACAT |
| 1751 | CGATATTGTC GTTTGAAAAC AACGAATGCC AGAATCAAGG |
| | AGGTGGGGCT |
| 1801 | TACGTTACTA AAACCTTCCA GTGTTCCGAT TCTCATCGCC |
| | TCCAGTTTAC |
| 1851 | TAGTAATAAA GCAGCAGATG AAGGCGGGGG CCTGTATTGT |
| | CGTGACGATG |
| 1901 | TCACGCTAAC GAACCTGACA GGGAAAACAC TATTTCAAGA |
| | GAATAGCAGT |
| 1951 | GAGAAACATG GAGGTGGGCT CTCTCTCGCC TCAGGAAAAT |
| | CTCTGACTAT |
| 2001 | GACATCGTTA GAGAGCTTCT GCTTAAATGC AAATACAGCA |
| | AAGGAAAACG |
| 2051 | GAGGCGGTGC GAATGTCCCT GAAAATATTG TACTCACCTT |
| | CACCTATACT |
| 2101 | CCCACTCCAA ATGAACCTGC GCCTGTGCAG CAGCCCGTGT |
| | ATGAGGAAGC |
| 2151 | TCTTGTTACT GGAAATACAG CCACAAAAAG TGGTGGGGGC |
| | ATTTACACGA |
| 2201 | AAAATGCGGC CTTCTCAAAT TTATCTTCTG TAACTTTTGA |
| | TCAAAATACC |
| 2251 | TCTTCAGAAA ATGGTGGTGC CTTACTTACC CAAAAAGCTG |
| | CAGATAAAAC |
| 2301 | GGACTGTTCT TTCACCTATA TTACAAATGT CAATATCACC |
| | AACAATACAG |
| 2351 | CTACAGGAAA TGGTGGGGGC ATTGCTGGGG GAAAAGCACA |
| | TTTCGATCGC |
| 2401 | ATTGATAATC TTACAGTCCA AAGCAACCAA GCAAAGAAAG |
| | GTGGTGGGGT |
| 2451 | TTATCTTGAA GATGCCCTCA TCCTGGAAAA GGTTATTACA |
| | GGTTCTGTCT |
| 2501 | CACAAAATAC AGCTACAGAA AGTGGTGGGG GTATCTACGC |
| | TAAGGATATT |
| 2551 | CAACTACAAG CTCTACCTGG AAGCTTCACA ATTACCGATA |
| | ATAAAGTCGA |
| 2601 | AACTAGTCTT ACTACTAGCA CTAATTTATA TGGTGGGGGC |
| | ATCTATTCCA |
| 2651 | GTGGAGCTGT CACGCTAACC AATATATCTG GAACCTTTGG |
| | CATTACAGGA |
| 2701 | AACTCTGTTA TCAATACAGC GACATCCCAG GATGCAGATA |
| | TACAAGGTGG |
| 2751 | GGGCATTTAT GCAACCACGT CTCTCTCAAT AAATCAATGT |
| | AATACACCCA |
| 2801 | TTCTATTTAG CAACAACTCT GCTGCCACTA AAAAAACATC |
| | AACAACAAAG |
| 2851 | CAAATTGCTG GTGGGGCTAT CTTCTCCGCT GCAGTAACTA |
| | TCGAGAATAA |
| 2901 | CTCTCAGCCC ATTATTTTCT TAAATAATTC CGCAAAGTCG |
| | GAAGCAACTA |
| 2951 | CAGCAGCAAC TGCAGGAAAT AAAGATAGCT GTGGAGGAGC |
| | CATTGCAGCT |
| 3001 | AACTCTGTTA CTTTAACAAA TAACCCTGAA ATAACCTTTA |
| | AAGGAAATTA |
| 3051 | TGCAGAAACT GGAGGAGCGA TTGGCTGTAT TGATCTTACT |
| | AATGGCTCAC |
| 3101 | CTCCCCGTAA AGTCTCTATT GCAGACAACG GTTCTGTCCT |
| | TTTTCAAGAC |
| 3151 | AACTCTGCGT TAAATCGCGG AGGCGCTATC TATGGAGAGA |
| | CTATCGATAT |
| 3201 | CTCCAGGACA GGTGCGACTT TCATCGGTAA CTCTTCAAAA |
| | CATGATGGAA |
| 3251 | GTGCAATTTG CTGTTCAACA GCCCTAACTC TTGCGCCAAA |
| | CTCCCAACTT |
| 3301 | ATCTTTGAAA ACAATAAGGT TACGGAAACC ACAGCCACTA |
| | CAAAAGCTTC |
| 3351 | CATAAATAAT TTAGGAGCTG CAATTTATGG AAATAATGAG |
| | ACTAGTGACG |
| 3401 | TCACTATCTC TTTATCAGCT GAGAATGGAA GTATTTTCTT |
| | TAAAAACAAT |
| 3451 | CTATGCACAG CAACAAACAA ATACTGCAGT ATTGCTGGAA |
| | ACGTAAAATT |
| 3501 | TACAGCAATA GAAGCTTCAG CAGGGAAAGC TATATCTTTC |
| | TATGATGCAG |
| 3551 | TTAACGTTTC CACCAAAGAA ACAAATGCTC AAGAGCTAAA |
| | ATTAAATGAA |
| 3601 | AAAGCGACAA GTACAGGAAC GATTCTATTT TCTGGGGAAC |
| | TTCACGAAAA |
| 3651 | TAAATCCTAT ATTCCACAGA AAGTCACTTT CGCACATGGG |
| | AATCTCATTC |
| 3701 | TAGGTAAAAA TCGAGAACTT AGCGTAGTTT CCTTTACCCA |
| | ATCTCCAGGC |
| 3751 | ACCACAATCA CTATGGGCCC AGGATCGGTT CTTTCCAACC |
| | ATAGCAAAGA |
| 3801 | AGCAGGAGGA ATCGCTATAA ACAATGTCAT CATTGATTTT |
| | AGTGAAATCG |
| 3851 | TTCCTACTAA AGATAATGCA ACAGTAGCTC CACCCACTCT |
| | TAAATTAGTA |
| 3901 | TCGAGAACTA ATGCAGATAG TAAAGATAAG ATTGATATTA |
| | CAGGAACTGT |
| 3951 | GACTCTTCTA GATCCTAATG GCAACTTATA TCAAAATTCT |
| | TATCTTGGTG |
| 4001 | AAGACCGCGA TATCACTCTT TTCAATATAG ACAATTCTGC |
| | AAGTGGGGCA |
| 4051 | GTTACAGCCA CGAATCTCAC CCTTCAAGGG AATTTAGGAG |
| | CTAAAAAAGG |
| 4101 | ATATTTAGGA ACCTGGAATT TGGATCCAAA TTCCTCGGGT |
| | TCAAAAATTA |
| 4151 | TTCTAAAATG GACCTTTGAC AAATACCTGC GCTGGCCCTA |
| | CATCCCTAGA |
| 4201 | GACAACCACT TCTACATCAA CTCTATTTGG GGAGCACAAA |
| | ACTCTTTAGT |
| 4251 | GACTGTGAAA CAAGGGATCT TAGGGAACAT GTTGAACAAT |
| | GCAAGGTTTG |
| 4301 | AAGATCCTGC TTTCAACAAC TTCTGGGCTT CGGCTATAGG |
| | ATCTTTCCTT |
| 4351 | AGGAAAGAAG TATCTCGAAA TTCTGACTCA TTCACCTATC |
| | ATGGCAGAGG |
| 4401 | CTATACCGCT GCTGTGGATG CCAAACCTCG CCAAGAATTT |
| | ATTTTAGGAG |
| 4451 | CTGCCTTCAG TCAGGTTTTT GGTCACGCCG AGTCTGAATA |
| | TCACCTTGAC |
| 4501 | AACTATAAGC ATAAAGGCTC AGGTCACTCT ACACAAGCAT |
| | CTCTTTATGC |
| 4551 | TGGCAATATC TTCTATTTTC CTGCGATACG GTCTCGGCCT |
| | ATTCTATTCC |
| 4601 | AAGGTGTGGC GACCTATGGT TATATGCAAC ATGACACCAC |
| | AACCTACTAT |
| 4651 | CCTTCTATTG AAGAAAAAAA TATGGCAAAC TGGGATAGCA |
| | TTGCTTGGTT |
| 4701 | ATTTGATCTG CGTTTCAGTG TGGATCTTAA AGAACCTCAA |
| | CCTCACTCTA |
| 4751 | CAGCAAGGCT TACCTTCTAT ACAGAAGCTG AGTATACCAG |
| | AATTCGCCAG |
| 4801 | GAGAAATTCA CAGAGCTAGA CTATGATCCT AGATCTTTCT |
| | CTGCATGCTC |
| 4851 | TTATGGGAAC TTAGCAATTC CTACTGGATT CTCTGTAGAC |
| | GGAGCATTAG |
| 4901 | CTTGGCGTGA GATTATTCTA TATAATAAAG TATCAGCTGC |
| | GTACCTCCCT |
| 4951 | GTGATTCTCA GGAATAATCC AAAAGCGACC TATGAAGTTC |
| | TCTCTACAAA |
| 5001 | AGAAAAGGGC AACGTAGTCA ACGTTCTCCC TACAAGAAAC |
| | GCAGCTCGTG |
| 5051 | CAGAGGTGAG CTCTCAAATT TATCTTGGAA GTTACTGGAC |
| | ACTCTACGGC |
| 5101 | ACGTATACTA TTGATGCTTC AATGAATACT TTAGTGCAAA |
| | TGGCCAACGG |
| 5151 | AGGGATCCGG TTTGTATTCT AG |

The PSORT algorithm predicts an outer membrane location (0.926).

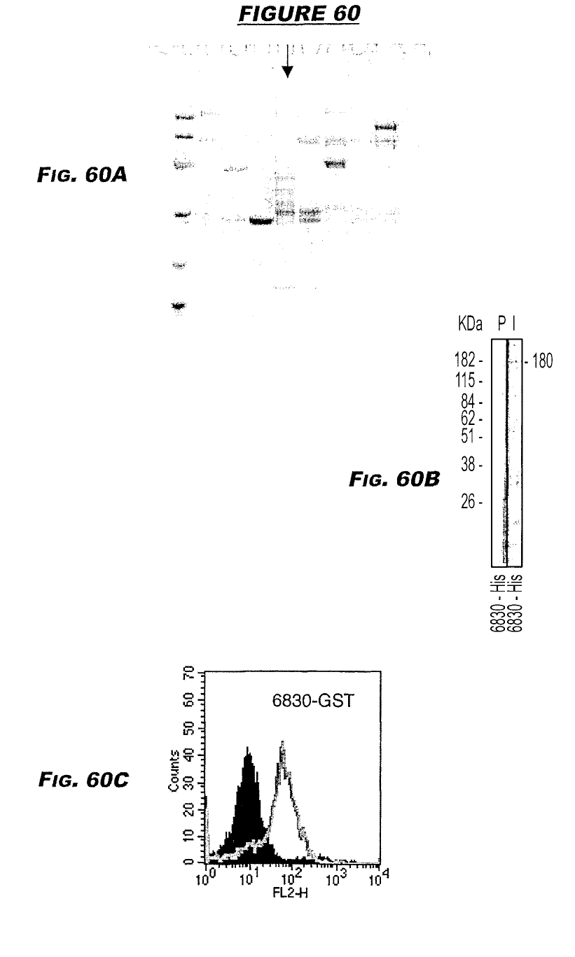

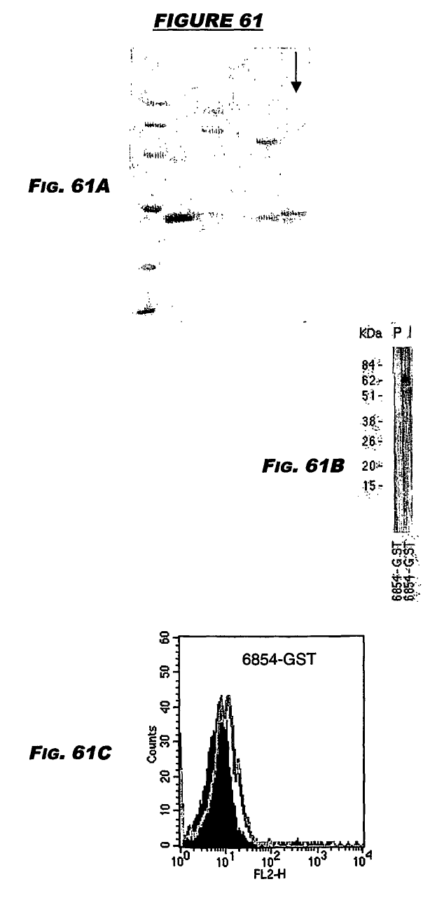

The protein was expressed in E. coli and purified as a GST-fusion (FIG. 60A) or his-tagged product. The recombinant proteins were used to immunise mice, whose sera were used in Western blot (FIG. 60B) and FACS (FIG. 60C) analyses.

The cp6830 protein was also identified in the 2D-PAGE experiment (Cpn0540) and showed good cross-reactivity with human sera, including sera from patients with pneumonitis.

These experiments show that cp6830 is a surface-exposed and immunoaccessible protein, and that it is a useful immunogen. These properties are not evident from the sequence alone.

### Example 61

The following C. pneumoniae protein (PID 4376854) was expressed <SEQ ID 121; cp6854>:

| | |
|---|---|
| 1 | MSIAIAREQY AAILDMHPKP SIAMFSSEQA RTSWEKRQAH |
| | PYLYRLLEII |
| 51 | WGVVKFLLGL IFFIPLGLFW VLQKICQNFI LLGAGGWIFR |
| | PICRDSNLLR |
| 101 | QAYAARLFSA SFQDHVSSVR RVCLQYDEVF IDGLELRLPN |
| | AKPDRWMLIS |
| 151 | NGNSDCLEYR TVLQGEKDWI FRIAEESQSN ILIFNYPGVM |
| | KSQGHITRNN |
| 201 | VVKSYQACVR YLRDEPAGPQ ARQIVAYGYS LGASVQAEAL |
| | SKEIADGSDS |
| 251 | VRWFVVKDRG ARSTGAVAKQ FIGSLGVWLA NLTEWNINSE |
| | KRSKDLHCPE |
| 301 | LFIYGKDSQG NLIGDGLFKK ETCFAAPFLD PKNLEECSGK |
| | KIPVAQTGLR |
| 351 | EDHILSDDVI KEVAGHIQRH FDN* |

The cp6854 nucleotide sequence <SEQ ID 122> is:

| | |
|---|---|
| 1 | ATGTCAATAG CTATTGCAAG GGAACAATAC GCAGCTATAT |
| | TGGATATGCA |
| 51 | TCCTAAACCT TCGATCGCCA TGTTTTCTTC GGAGCAGGCG |
| | AGAACTTCTT |
| 101 | GGGAGAAACG ACAGGCTCAT CCTTACCTTT ATCGTCTTCT |
| | TGAGATCATA |
| 151 | TGGGGTGTTG TGAAATTTCT TCTCGGCTTA ATCTTCTTTA |
| | TTCCCTTGGG |
| 201 | TCTTTTCTGG GTCCTTCAGA AGATATGTCA GAATTTTATT |
| | CTTCTTGGTG |
| 251 | CAGGAGGGTG GATTTTTAGA CCCATATGCA GGGACTCTAA |
| | TTTATTGCGA |
| 301 | CAAGCTTACG CCGCGCGTCT TTTCTCCGCT TCATTCCAAG |
| | ATCATGTCTC |
| 351 | CTCTGTGCGA AGGGTTTGCT TACAGTATGA CGAGGTCTTT |
| | ATTGACGGAT |
| 401 | TGGAGTTACG TCTTCCCAAT GCTAAGCCAG ATCGATGGAT |
| | GTTAATCTCC |
| 451 | AATGGAAACT CCGATTGCTT AGAGTATAGG ACAGTGCTGC |
| | AAGGGGAAAA |
| 501 | GGACTGGATA TTCCGTATTG CTGAAGAGTC TCAATCCAAC |
| | ATTTTAATCT |
| 551 | TCAATTACCC AGGAGTCATG AAGAGCCAAG GGAATATAAC |
| | AAGAAACAAT |
| 601 | GTAGTCAAAT CTTATCAAGC ATGCGTACGC TATCTTAGAG |
| | ATGAACCCGC |
| 651 | AGGACCTCAG GCGCGTCAAA TCGTTGCTTA TGGCTATTCT |
| | TTAGGAGCTA |
| 701 | GTGTTCAAGC CGAAGCATTA AGTAAAGAGA TCGCAGACGG |
| | AAGTGATAGC |
| 751 | GTCCGTTGGT TTGTCGTTAA AGATCGAGGA GCTCGCTCTA |
| | CAGGAGCCGT |
| 801 | TGCTAAACAG TTTATTGGAA GTCTAGGAGT TTGGCTGGCG |
| | AATCTTACCC |
| 851 | ATTGGAATAT TAATTCTGAA AAGAGAAGCA AGGACTTGCA |
| | TTGCCCAGAA |
| 901 | CTCTTTATTT ATGGCAAGGA TTCCCAAGGT AATCTTATCG |
| | GGGATGGATT |
| 951 | GTTCAAAAAA GAGACGTGCT TCGCAGCACC ATTTTTAGAT |
| | CCTAAAAACT |
| 1001 | TGGAAGAGTG TTCAGGGAAG AAAATCCCTG TAGCTCAGAC |
| | CGGTCTAAGA |
| 1051 | CACGATCATA TCCTTTCCGA TGATGTGATT AAAGAAGTTG |
| | CAGGTCATAT |
| 1101 | TCAAAGACAT TTCGATAATT A |

The PSORT algorithm predicts an inner membrane location (0.461).

The protein was expressed in E. coli and purified as a GST-fusion product, as shown in FIG. 61A. The recombinant protein was used to immunise mice, whose sera were used in Western blot (FIG. 61B) and FACS (FIG. 61C) analyses. A his-tagged protein was also expressed.

These experiments show that cp6854 is a surface-exposed and immunoaccessible protein, and that it is a useful immunogen. These properties are not evident from the sequence alone.

### Example 62

The following C. pneumoniae protein (PID 4377101) was expressed <SEQ ID 123; cp7101>:

| | |
|---|---|
| 1 | MYSCYSKGIS HNYLIHPMSR LDIFVFDSLI ANQDQNLLEE |
| | IFCSEDTVLF |
| 51 | KAYRTTALQS PLAAKNLNIA RKVANYILAD NGEIDTVKLV |
| | EAIHHLSQCT |
| 101 | YPLGPHRHNE AQDREHLLKM LKALKENPKL KESIKTLFVP |
| | SYSTIQNLIR |
| 151 | HTLALNPQTI LSTIHVRQAA LTALFTYLRQ DVGSCFATAP |
| | AILIHQEYPE |
| 201 | RFLKDLNDLI SSGKLSRIVN QREIAVPINL SGCIGELFKP |
| | LRILDLYPDP |
| 251 | LVKLSSSPGL KKAFSAANLI ETLGDSAEQI QQLLSHQYLM |
| | QKLQNVHETL |
| 301 | TANDIIKSTL LHYYQLQEST VRAIFFKEGL FSKEQVAFST |
| | QHPRFLSSIQ |
| 351 | RVYHYLHAYE EAKSAFIHDT QNPLLKAWEY TLATLADASQ |
| | PTISNHIRLA |
| 401 | LGWKSEDPHS LVSLVTHFVE EEVENIRIDV QQCEQTYEEA |
| | RSQLEYIEGR |
| 451 | MRNPLNNQDS QILTMDHMRF RQELNKALYE WSDAQEKAKK |
| | FLHLPEFLLS |
| 501 | FYTKQIPLYF RSSYDAFIQE FAHLYANAPA GFRILFTHGR |
| | THPNTWSPIY |
| 551 | SINEFIRFLS EFFTSTESEL LSKHAVINLE KETSRLVENI |
| | TAMLHTDVFQ |
| 601 | EALLTRIIEA YQLPVPPSIL NHLDQLSQTP WVYVSGGTVD |
| | TLLLDYFESS |
| 651 | EPLTLTEKHP ENPHELAAFY ADALKDLPTG IKSYLEEGSH |
| | SLLSSSPTHV |
| 701 | FSIIAGSPLF REAWDNDWYS YTWLRDVWVK QHQDFLQDTI |
| | LPQLSIYAFI |
| 751 | ENFCNKYALQ HVVHDFHDFC SDHSLTLPEL YDKGSRFLSS |
| | LFTKDKTVAL |
| 801 | IYIRRLLYLM VREVPYVSEQ QLPEVLDNVS SYLGISSRIT |
| | YEKFRSLIEE |
| 851 | TIPKMTLISS ADLRHIYKSL LMQSYQKIYT EEDTYLRLTT |
| | AMRHHNLAYP |
| 901 | APLLFADSNW PSIYFGFILN PGTTEIDLWK FNYAGLQGQP |
| | LDNIQELFAT |
| 951 | SRPWTLYANP IDYGMPPPPG YRSRLPKEFF * |

The cp7101 nucleotide sequence <SEQ ID 124> is:

| | |
|---|---|
| 1 | ATGTATTCGT GTTACAGCAA AGGAATATCC GATAACTATC |
| | TTCTACATCC |
| 51 | TATGTCACGT TTGGATATTT TTGTTTTCGA TTCTCTGATC |
| | GCAAACCAGG |
| 101 | ATCAAAATCT TCTTGAGGAA ATTTTCTGTT CTGAAGACAC |
| | AGTTTTATTT |
| 151 | AAGCCTACC GTACTACGGCC TCTACAATCC CCTCTAGCTG |
| | CTAAGAACCT |
| 201 | AAATATCGCC CGTAAAGTCG CAAATTATAT CTTAGCTGAC |
| | AATGGGGAAA |
| 251 | TCGATACAGT AAAGCTTGTC GAAGCCATTC ACCATCTCTC |
| | ACAATGTACC |
| 301 | TATCCTTTAG GGCCTCATCG CCATAATGAA GCTCAAGATC |
| | GTGAACACCT |
| 351 | CCTTAAAATG CTAAAAGCTC TAAAGGAAAA TCCTAAATTA |
| | AAAGAAAGCA |
| 401 | TCAAAACTCT CTTTGTCCCT TCATACTCTA CAATCCAAAA |
| | CCTAATTCGC |
| 451 | CATACACTAG CATTGAATCC ACAGACAATT CTCTCTACGA |
| | TTCATGTGCG |
| 501 | TCAAGCAGCA CTCACAGCGC TCTTCACCTA CCTTCGGCAA |
| | GATGTAGGTT |
| 551 | CCTGTTTTGC TACGGCTCCT GCCATTCTCA TTCACCAAGA |
| | ATATCCAGAA |
| 601 | CGATTCCTTA AAGATCTCAA TGATCTCATT AGCATGGGCA |
| | AACTCTCTAG |
| 651 | AATCGTAAAC CAAAGGGAAA TTGCGGTTCC TATAAACCTT |
| | TCGGGATGCA |
| 701 | TTGGAGAGCT ATTCAAGCCT TTAAGGATTC TAGATCTTTA |
| | TCCTGATCCT |
| 751 | CTGGTTAAGC TCTCCTCATC TCCAGGACTC AAAAAAGCCT |
| | TTTCTGCTGC |
| 801 | CAATCTTATT GAAACTCTTG GGGATTCTGA AGCACAAATC |
| | CAACAGTTGC |
| 851 | TCTCGCATCA ATATTTGATG CAAAAACTAC AAAATGTCCA |
| | TGAGACCTTA |
| 901 | ACTGCTAACG ACATTATCAA ATCGACACTT CTGCACTACT |
| | ATCAGCTCCA |
| 951 | AGAAAGTACT GTACGAGCTA TTTTCTTCAA AGAAGGGTTG |
| | TTCAGCAAAG |
| 1001 | AACAAGTGGC ATTCTCGACG CAACACCCCA GAGAGCTCTC |
| | AGAAATACAA |
| 1051 | CGGGTATACC ACTACTTACA TGCCTATGAA GAAGCAAAAT |
| | CTGCTTTTAT |
| 1101 | CCATGACACT CAAAATCCCT TACTGAAAGC CTGGGAGTAT |
| | ACTTTAGCGA |
| 1151 | CTCTTGCGGA TGCTAGCCAA CCTACCATCT CAAACCATAT |
| | CCGCCTTGCC |
| 1201 | TTAGGATGGA AAAGTGAAGA CCCTCACAGT CTTGTATCTC |
| | TAGTTACACA |
| 1251 | CTTTGTTGAA GAGGAAGTAG AAAACATCCG AATTTTAGTC |
| | CAACAATGTG |
| 1301 | AACAGACCTA TCACGAAGCA CGCTCCCAAC TAGAATATAT |
| | TGAAGGGCGG |
| 1351 | ATGCGCAACC CACTAAATAA TCAAGACAGT CAGATTTTGA |
| | CGATGGATCA |
| 1401 | CATGCGCTTC CGTCAAGAAC TCAATAAAGC TCTTTATGAG |
| | TGGGATAGTG |
| 1451 | CTCAAGAAAA GGCAAAGAAA TTTCTACATC TTCCTGAATT |
| | CTTACTTTCT |
| 1501 | TTCTATACAA AGCAAATTCC CTTATACTTT CGTAGTTCTT |
| | ACGATGCCTT |
| 1551 | CATTGAAGAA TTTGCTCATC TCTATGCTAA TGCTCCCGCT |
| | GGCTTCCGTA |
| 1601 | TTCTTTTCAC GCATGGACGC ACCCATCCGA ACACATGGTC |
| | CCCCATCTAT |
| 1651 | TCGATTAATG AATTTATACG TTTTCTTTCT GAATTCTTCA |
| | CCTCCACAGA |
| 1701 | GTCAGAACTT CTGGGGAAAC ATGCCGTGAT CAATTTAGAG |
| | AAAGAAACAT |
| 1751 | CTCGGCTCGT CCACAACATC ACTGCCATGC TACACACGGA |
| | TGTTTTCCAA |
| 1801 | GAAGCTCTCC TTACAAGAAT TTTAGAAGCC TATCAGCTTC |
| | CTGTGCCTCC |
| 1851 | CTCCATCTTA AACCACTTAG ATCAGCTGTC ACAAACTCCC |
| | TGGGTTTATG |
| 1901 | TTTCTGGAGG AACAGTGGAC ACTCTTCTTT TGGATTATTT |
| | TGAAAGCTCA |
| 1951 | GAACCTCTGA CACTTACAGA AAAGCATCCT GAAAATCCTC |
| | ATGAGCTTGC |
| 2001 | AGCTTTCTAC GCAGACGCCC TTAAAGATCT CCCTACAGGA |
| | ATTAAAAGTT |
| 2051 | ATCTAGAAGA AGGATCCCAC TCTCTACTTA GCTCATCACC |
| | CACCCACGTT |
| 2101 | TTCTCTATAA TCGCAGGATC TCCTTTATTT CGGGAAGCTT |
| | GGGATAATGA |
| 2151 | TTGGTACAGC TATACCTGGC TTCGTGATGT CTGGGTGAAA |
| | CAACACCAAG |
| 2201 | ATTTCCTTCA AGATACTATA TTACCTCAGC TAAGTATCTA |
| | TGCTTTCATA |
| 2251 | GAGAATTTTT GTAACAAATA TGCTTTGCAA CATGTAGTTC |
| | ATGACTTTCA |
| 2301 | TGATTTCTGC TCCGACCACT CCTTGACTCT TCCGGAGCTC |
| | TATGACAAAG |
| 2351 | GATCGCGTTT TCTAAGCTCC TTATTCACCA AAGATAAGAC |
| | CGTAGCTCTT |
| 2401 | ATCTATATAC GCCGTCCTCT CTACCTTATG GTCCGTGAAG |
| | TCCCTTATGT |
| 2451 | TTCAGAACAA CAGCTTCCAG AAGTCTTAGA TAACGTCTCT |
| | TCATATCTCG |
| 2501 | GGATTTCCTA TCGTATTACC TATGAGAAAT TCCGCTCCCT |
| | GATAGAGGAA |
| 2551 | ACCATCCCTA AAATGACCTT ACTCTCCTCA GCAGACCTGA |
| | GGCATATCTA |
| 2601 | TAAAGGTCTC CTCATGCAAA GTTATCAAAA GATCTACACC |
| | GAAGAAGATA |
| 2651 | CGTACCTCCG CCTCACCACG GCAATGAGGC ATCATAATCT |
| | TGCCTATCCC |
| 2701 | GCTCCTTTGC TCTTTGCAGA CAGTAACTGG CCTTCTATTT |
| | ATTTTGGATT |
| 2751 | CATCCTAAAT CCAGGAACCA CAGAGATCGA TCTTTGGAAA |
| | TTTAACTATG |
| 2801 | CAGGGCTGCA AGGACAGCCT CTTGACAATA TCCAGGAGCT |
| | GTTCGCAACG |
| 2851 | TCAAGACCCT GGACCCTCTA TGCAAATCCT ATAGATTATG |
| | GCATGCCACC |
| 2901 | GCCTCCAGGC TACCGCAGCC GCCTCCCTAA AGAATTTTTC |
| | TAG |

The PSORT algorithm predicts a cytoplasmic location (0.206).

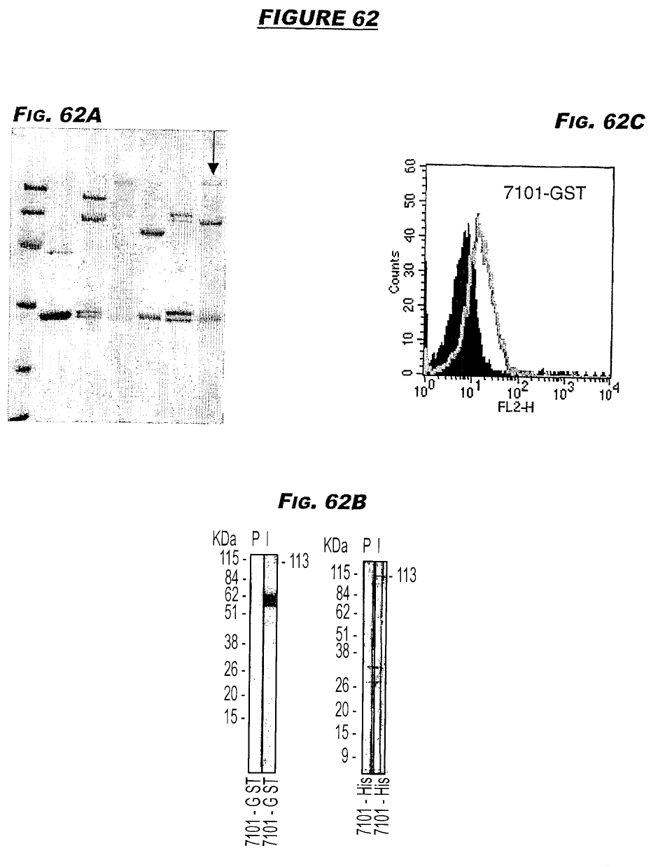

The protein was expressed in E. coli and purified as a GST-fusion (FIG. 62A) or his-tagged product. The proteins were used to immunise mice, whose sera were used in Western blot (FIG. 62B) and FACS (FIG. 62C) analyses.

This protein also showed good cross-reactivity with human sera, including sera from patients with pneumonitis.

These experiments show that cp7101 is a surface-exposed and immunoaccessible protein, and that it is a useful immunogen. These properties are not evident from the sequence alone.

### Example 63

The following C. pneumoniae protein (PID 4377107) was expressed <SEQ ID 125; cp7107>:

| | |
|---|---|
| 1 | MSIVRNSALP LPCLSRSETF KKVRSHMKFM KVLTPWIYRK |
| | DLWVTAFLLT |
| 51 | AIPGSFAHTL VDIAGERRHA AQATGVSGDG KIVIGMKVPD |
| | DPFAITVGFQ |
| 101 | YIDGHLQPLE AVRPQCSVYP NGITPDGTVI VGTNYAIGMG |
| | SVAVKWVNGK |
| 151 | VSELPMLPDT LDSVASAVSA DGRVIGGNRN INLGASVAVK |
| | WEDDVITQLP |
| 201 | SLPDAMNACV NGISSDGSII VGTMVDVSWR NTAVQWIGDQ |
| | LSVIGTLGGT |
| 251 | TSVASAISTD GTVIVGGSEN ADSQTHAYAY KNGVMSDIGT |
| | LGGFYSLAHA |
| 301 | VSSDGSVIVG VSTNSERRYH AFQYADGQMV DLGTLGGPES |
| | YAQGVSGDGK |
| 351 | VIVGRAQVPS GDWHAFLCPF QAPSPAPVHG GSTVVTSQNP |
| | RGMVDINATY |
| 401 | SSLKNSQQQL QRLLIQHSAK VESVSSGAPS FTSVKGAISK |
| | QSPAVQNDVQ |
| 451 | KGTFLSYRSQ VHGNVQNQQL LTGAFMDWKL ASAPKCGFKV |
| | ALHYGSQDAL |
| 501 | VERAALPYTE QGLGSSVLSG FGGQVQGRYD FNLGETVVLQ |
| | PFMGIQVLHL |
| 551 | SREGYSEKNV RFPVSYDSVA YSAATSFMGA HVFASLSPKM |
| | STAATLGVER |
| 601 | DLNSHIDEFK GSVSAMGNFV LENSTVSVLR RFASLAMYYD |
| | VRQQQLVTLS |
| 651 | VVMNQQPLTG TLSLVSQSSY NLSF* |

The cp7107 nucleotide sequence <SEQ ID 126> is:

| | |
|---|---|
| 1 | ATGAGTATAG TCAGAAATTC TGCATTGCCA CTTCCGTGTT |
| | TAAGCAGATC |
| 51 | CGAAACCTTT AAAAAAGTTA GGTCGCATAT GAAATTTATG |
| | AGAGTCCTTA |
| 101 | CTCCATGGAT TTATCGAAAA GATCTTTGGG TAACAGCATT |
| | CTTACTGACA |
| 151 | GCAATTCCAG GATCTTTTGC ACATACTCTT GTTGATATAG |
| | CAGGAGAACC |
| 201 | TCGGCATGCT GCTCAAGCAA CAGGAGTTTC TGGAGATGGT |
| | AAAATTGTTA |
| 251 | TAGGAATGAA AGTTCCGGAT GATCCTTTTG CTATAACTGT |
| | AGGATTTCAA |
| 301 | TATATTGATG GGCATTTGCA ACCCTTAGAG GCAGTACGTC |
| | CTCAATGCTC |
| 351 | TGTATACCCT AATGGTATAA CCCCGGACGG AACGGTTATT |
| | GTGGGTACAA |
| 401 | ACTATGCCAT CGGGATGGGT AGTGTTGCTG TGAAATGGGT |
| | AAATGGCAAG |
| 451 | GTTTCTGAAC TTCCCATGCT CCCTGACACC CTCGATTCTG |
| | TAGCATCGGC |
| 501 | AGTTTCTGCA GATGGAAGAG TGATTGGAGG GAATAGAAAT |
| | ATAAATCTTG |
| 551 | GCGCTTCTGT TGCTGTGAAA TGGGAGGACG ACGTGATTAC |
| | ACAACTTCCT |
| 601 | TCTCTTCCTG ATGCTATGAA TGCTTGTGTT AACGGAATTT |
| | CTTCAGATGG |
| 651 | TTCTATAATT GTAGGAACCA TGGTAGACGT GTCATGGAGA |
| | AATACCGCAG |
| 701 | TACAATGGAT CGGGGATCAG CTCTCTGTTA TTGGGACTTT |
| | AGGAGGAACT |
| 751 | ACTTCTGTTG CTAGTGCAAT CTCAACAGAT GGCACTGTGA |
| | TTGTAGGAGG |
| 801 | TTCTGAAAAT GCAGATTCTC AGACTCATGC CTATGCTTAT |
| | AAAAACGGTG |
| 851 | TTATGAGCGA TATAGGGACC CTCGGAGGTT TTTATTCTTT |
| | AGCACATGCA |
| 901 | GTATCTTCAG ATGGTTCTGT GATTGTAGGA GTATCCACGA |
| | ACTCTGAGCA |
| 951 | TAGATATCAT GCATTCCAAT ATGCTGATGG ACAGATGGTA |
| | GATTTAGGAA |
| 1001 | CTTTAGGAGG GCCTGAATCT TATGCTCAAG GTGTGTCTGG |
| | AGATGGAAAG |
| 1051 | GTAATTGTGG GTAGAGCACA AGTACCATCT GGAGATTGGC |
| | ATGCGTTCCT |
| 1101 | ATGTCCTTTC CAAGCTCCGA GCCCTGCTCC TGTCCATGGG |
| | GGAAGCACTG |
| 1151 | TCGTAACTAG CCAGAATCCA CGTGGAATGG TAGATATCAA |
| | TGCTACGTAC |
| 1201 | TCCTCTTTGA AAAATAGCCA ACAACAACTA CAAAGATTGC |
| | TTATCCAGCA |
| 1251 | TAGTGCAAAA GTTGAAAGTG TATCCTCAGG AGCACCATCT |
| | TTTACAAGTG |
| 1301 | TGAAAGGTGC GATCTCAAAA CAGAGCCCTG CAGTGCAAAA |
| | TGATGTACAG |
| 1351 | AAAGGGACGT TTTTAAGTTA CCGTTCCCAA GTTCATGGAA |
| | ACGTGCAGAA |
| 1401 | TCAGCAATTG CTCACAGGAG CTTTTATGGA CTGGAAACTC |
| | GCTTCAGCTC |
| 1451 | CTAAATGCGG CTTTAAAGTA GCTCTCCACT ATGGCTCTCA |
| | AGATGCTCTC |
| 1501 | GTAGAACGTG CAGCTCTTCC TTACACAGAA CAAGGCTTAG |
| | GAAGCAGTGT |
| 1551 | CTTGTCAGGT TTTGGAGGAC AAGTTCAAGG ACGCTATGAC |
| | TTTAATTTAG |
| 1601 | GAGAAACTGT TGTTCTGCAA CCCTTTATGG GCATTCAAGT |
| | TCTCCACCTA |
| 1651 | AGTAGAGAAG GGTATTCTGA GAAGAATGTT CGATTTCCTG |
| | TAAGCTATGA |
| 1701 | TTCTGTAGCC TACTCAGCAG CTACTAGCTT TATGGGTGCG |
| | CATGTATTTG |
| 1751 | CCTCCCTAAG CCCTAAAATG AGTACAGCAG CAACTTTAGG |
| | TGTGGAGAGA |
| 1801 | GATCTGAATT CACATATAGA TGAATTTAAG GGATCCGTCT |
| | CTGCTATGGG |
| 1851 | AAACTTTGTC TTGGAAAATT CTACAGTGAG TGTTTTAAGA |
| | CCTTTTGCTT |
| 1901 | CTCTTGCTAT GTACTATGAC GTAAGACAAC AGCAACTCGT |
| | GACGTTGTCA |
| 1951 | GTAGTTATGA ATCAACAACC CTTAACAGGC ACACTAAGCT |
| | TAGTAAGCCA |
| 2001 | AAGTAGCTAT AATCTTAGCT TCTAA |

The PSORT algorithm predicts an inner membrane location (0.100).

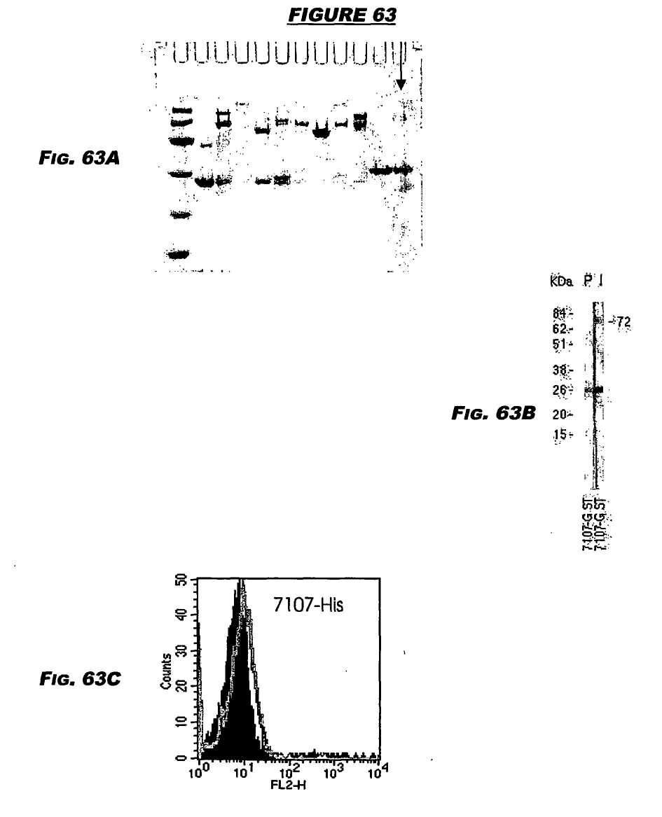

The protein was expressed in E. coli and purified as a GST-fusion (FIG. 63A) or his-tagged product. The proteins were used to immunise mice, whose sera were used in Western blot (FIG. 63B) and FACS (FIG. 63C) analyses.

These experiments show that cp7107 is a surface-exposed and immunoaccessible protein, and that it is a useful immunogen. These properties are not evident from the sequence alone.

### Example 64

The following C. pneumoniae protein (PID 4376467) was expressed <SEQ ID 127; cp6467>:

| | |
|---|---|
| 1 | MLRFFAVFIS TLWLITSGCS PSQSSKGIFV VNMKEMPRSL |
| | DPGKTRLIAD |
| 51 | QTLMRHLYEG LVEEHSQNGE IKPALAESYT ISEDGTRYTF |
| | KIKNILWSNG |
| 101 | DPLTAQDFVS SWKEILKEDA SSVYLYAFLP IKNARAIFDD |
| | TESPENLGVR |
| 151 | ALDKRHLEIQ LETPCAHFLH FLTLPIFFPV HETLRNYSTS |
| | FEEMPITCGA |
| 201 | FRPVSLEKGL RLHLEKNPMY HNKSRVKLHK IIVQFISNAN |
| | TAAILPKHKK |
| 251 | LDWQGPPWGE PIPPEISASL HQDDQLFSLP GASTTWLLFN |
| | IQKKPWNNAK |
| 301 | LRKALSLAID KDMLTKVVYQ GLAEPTDHIL HPRLYPGTYP |
| | ERKRQNERIL |
| 351 | EAQQLFEEAL DELQMTREDL EKETLTFSTF SFSYGRICQM |
| | LREQWKKVLK |
| 401 | FTIPIVGQEF FTIQKNFLEG NYSLTVNQWT AAFIDPMSYL |
| | MIFANPGGIS |
| 451 | PYHLQDSHFQ TLLIKITQEH KKHLRNQLII EALDYLEECH |
| | ILEPLCHPNL |
| 501 | RIALNKNIKN FNLFVRRTSD FRFIEKL* |

A predicted signal peptide is highlighted.

The cp6467 nucleotide sequence <SEQ ID 128> is:

| | |
|---|---|
| 1 | ATGCTCCGTT TCTTCGCTGT ATTTATATCA ACTCTTTGGC |
| | TCATTACCTC |
| 51 | AGGATGTTCC CCATCCCAAT CCTCTAAAGG AATTTTTGTG |
| | GTAAATATGA |
| 101 | AGGAAATGCC ACGCTCCTTG GATCCTGGAA AAACTCGTCT |
| | CATTGCAGAC |
| 151 | CAAACTCTAA TGCGTCATCT ATATGAAGGA CTCGTCGAAG |
| | AACATTCCCA |
| 201 | AAATGGAGAG ATTAAACCAG CCCTTGCAGA AAGCTACACC |
| | ATCTCCGAAG |
| 251 | ACGGGACTCG GTACACATTT AAAATCAAAA ACATCCTTTG |
| | GAGTAACGGA |
| 301 | GACCCTCTGA CAGCTCAAGA CTTTGTCTCC TCTTGGAAGG |
| | AAATCCTAAA |
| 351 | GGAAGATGCG TCCTCCGTAT ATCTCTATGC GTTTTTACCT |
| | ATCAAAAATG |
| 401 | CTCGGGCAAT CTTTGATGAT ACTGAGTCTC CAGAAAATCT |
| | AGGAGTCCGA |
| 451 | GCTTTAGATA AGCGTCATCT CGAAATTCAG TTAGAAACTC |
| | CCTGCGCGCA |
| 501 | TTTCCTACAT TTCTTGACTC TTCCTATTTT TTTCCCTGTT |
| | CATGAAACTC |
| 551 | TGCGAAACTA TAGCACCTCT TTTGAAGAGA TGCCCATTAC |
| | CTGCGGTGCT |
| 601 | TTCCGCCCTG TGTCTCTAGA AAAAGGCCTG AGACTCCATC |
| | TAGAGAAAAA |
| 651 | CCCTATGTAC CATAATAAAA GCCGTGTGAA ACTACATAAA |
| | ATTATTGTAC |
| 701 | AGTTTATCTC AAACGCTAAC ACTGCAGCCA TTCTATTCAA |
| | ACATAAGAAA |
| 751 | TTAGATTGGC AAGGACCTCC TTGGGGAGAA CCTATCCCTC |
| | CAGAAATCTC |
| 801 | AGCTTCTCTA CATCAAGATG ACCAGCTCTT TTCTCTTCCG |
| | CGCGCTTCGA |
| 851 | CTACATGGTT ACTCTTTAAT ATACAAAAAA AACCTTGGAA |
| | CAATGCTAAA |
| 901 | TTACGCAAGG CATTGAGCCT TGCAATAGAC AAAGATATGT |
| | TAACCAAAGT |
| 951 | GGTATACCAA GGTCTTGCAG AACCTACAGA TCATATCCTA |
| | CATCCAAGAC |
| 1001 | TTTATCCAGG GACCTATCCC GAACGGAAAA GACAAAACGA |
| | AAGAATTCTT |
| 1051 | GAGGCTCAAC AACTCTTTGA AGAAGCTCTA GACGAACTTC |
| | AAATGACACG |
| 1101 | CGAAGATCTA GAAAAGGAAA CTTTGACTTT CTCAACCTTT |
| | TCTTTTTCTT |
| 1151 | ACGGAAGGAT TTGCCAAATG CTAAGAGAAC AATGGAAGAA |
| | AGTCTTAAAA |
| 1201 | TTTACTATCC CTATAGTAGG CCAAGAGTTT TTCACAATAC |
| | AAAAAAACTT |
| 1251 | CCTAGAGGGG AACTATTCCC TAACCGTGAA CCAATGGACC |
| | GCAGCATTTA |
| 1301 | TTGATCCGAT GTCTTATCTC ATGATCTTTG CCAATCCTGG |
| | AGGAATTTCC |
| 1351 | CCCTATCACC TCCAAGATTC ACACTTTCAA ACTCTTCTCA |
| | TAAAGATCAC |
| 1401 | TCAAGAACAT AAAAAACACC TACGAAATCA GCTTATTATT |
| | GAAGCCCTTG |
| 1451 | ACTATTTAGA ACACTGTCAC ATTCTCGAAC CACTATGTCA |
| | TCCAAATCTT |
| 1501 | CGAATTGCTT TGAACAAAAA CATTAAAAAC TTTAATCTTT |
| | TTGTTCGACG |
| 1551 | AACTTCAGAC TTTCGTTTTA TAGAAAAACT ATAG |

The PSORT algorithm predicts an outer membrane lipoprotein (0.790).

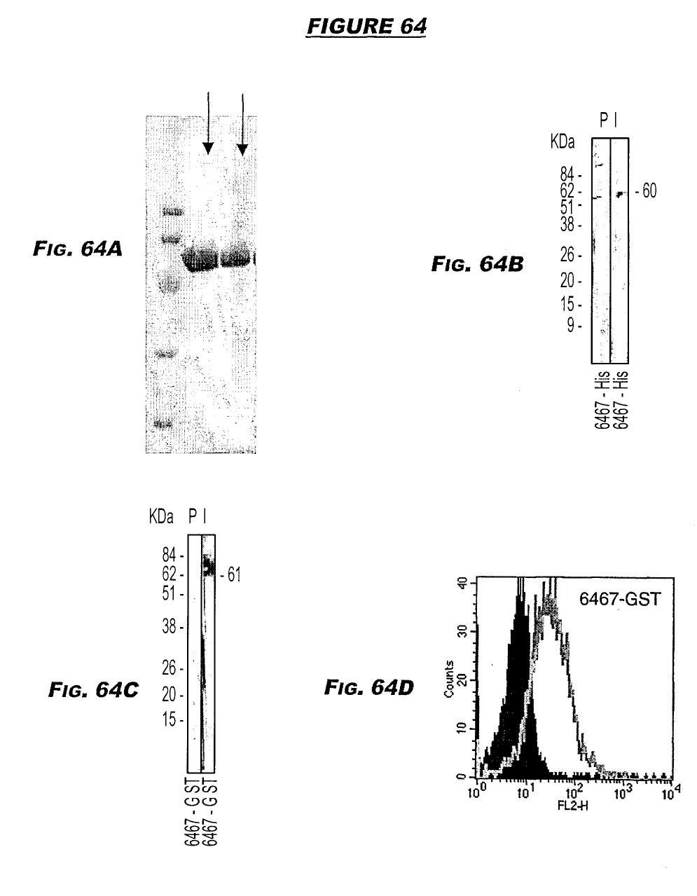

The protein was expressed in E. coli and purified as a his-tag product and a GST-fusion protein, as shown in FIG. 64A. The recombinant his-tag protein was used to immunise mice, whose sera were used in a Western blot (FIG. 64B). The recombinant GST-fusion protein was also used to immunise mice, whose sera were used in a Western blot (FIG. 64C) and for FACS analysis (FIG. 64D).

These experiments show that cp6467 is a surface-exposed and immunoaccessible protein, and that it is a useful immunogen. These properties are not evident from the sequence alone.

### Example 65

The following C. pneumoniae protein (PID 4376679) was expressed <SEQ ID 129; cp6679>:

| | |
|---|---|
| 1 | MRKMLVLLAS LGLLSPTLSS CTHLGSSGSY HPKLYTSGSK |
| | TKGVIAMLPV |
| 51 | FHRPGKSLEP LPWNLQGEFT EEISKRFYAS EKVFLIKHNA |
| | SPQTVSQFYA |
| 101 | PIANRLPETI IEQFLPAEFI VATELLEQKT GKEAGVDSVT |
| | ASVRVRVFDI |
| 151 | RHHKIALIYQ EIIECSQPLT TLVNDYHRYG WNSKHFDSTP |
| | MGLMHSRLFR |
| 201 | EVVARVEGYV CANYS* |

A predicted signal peptide is highlighted.

The cp6679 nucleotide sequence <SEQ ID 130> is:

| | |
|---|---|
| 1 | ATGCGAAAAA TGTTGGTATT ATTGGCATCT TTAGGACTTC |
| | TATCCCCAAC |
| 51 | CCTATCCAGC TCCACTCACT TAGGCTCTTC AGGAAGTTAT |
| | CATCCTAAGC |
| 101 | TATACACTTC AGGGAGCAAA ACTAAAGGTG TGATTGCGAT |
| | GCTTCCTGTA |
| 151 | TTTCATCGCC CAGGAAAGAG TCTTGAACCT TTACCTTGGA |
| | ACCTCCAAGG |
| 201 | AGAATTTACT GAAGAGATCA GCAAAAGGTT TTATGCTTCG |
| | GAAAAGGTCT |
| 251 | TCCTGATCAA GCACAATGCT TCACCTCAGA CAGTCTCTCA |
| | GTTCTATCCT |
| 301 | CCGATTGCGA ATCGTCTACC CGAAACAATT ATTGAGCAAT |
| | TTCTTCCTGC |
| 351 | AGAATTCATT GTTGCTACAG AACTGTTAGA ACAAAAGACA |
| | GGGAAAGAAG |
| 401 | CAGGTGTCGA TTCTGTAACA GCGTCTGTAC GTGTTCGCGT |
| | TTTTGATATC |
| 451 | CGTCATCATA AAATAGCTCT CATTTATCAA GAGATTATCG |
| | AATGCAGCCA |
| 501 | GCCTTTAACT ACCCTAGTCA ATGATTATCA TCGCTATGGC |
| | TGGAACTCAA |
| 551 | AACATTTTGA TTCAACGCCC ATGGGCTTAA TGCATAGCCG |
| | TCTTTTCCGC |
| 601 | GAAGTTGTTG CCAGAGTTGA GGGCTATGTT TGTGCTAACT |
| | ACTCGTAG |

The PSORT algorithm predicts an inner membrane location (0.149).

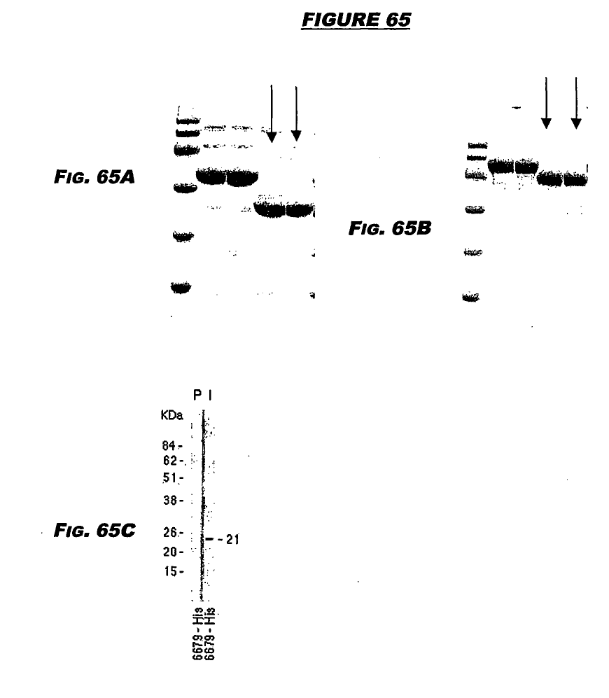

The protein was expressed in E. coli and purified as a his-tag product (FIG. 65A) and as a GST-fusion product (FIG. 65B). The recombinant protein was used to immunise mice, whose sera were used in a Western blot (FIG. 65C) and for FACS analysis.

These experiments show that cp6679 is a surface-exposed and immunoaccessible protein, and that it is a useful immunogen. These properties are not evident from the sequence alone.

### Example 66

The following C. pneumoniae protein (PID 4376890) was expressed <SEQ ID 131; cp6890>:

| | |
|---|---|
| 1 | MKQLLFCVCV FAMSCSAYAS RRRQDRSVMK ETFRNNYGII |
| | VSGQEWVKRG |
| 51 | SDGTITKVLK NGATLHEVYS GGLLHGEITL TFPHTTALDV |
| | VQIYDQGRLV |
| 101 | SRKTFFVNGL PSQEELFNED GTFVLTRWPD NNDSDTITKP |
| | YFIETTYQGH |
| 151 | VIEGSYISFN GKYSSSIHNG EGVRSVFSSN NILLSEETFN |
| | EGVMVKYTTF |
| 201 | YRNRDPESIT HYQNGQRHGL RLTYLQGGIP NTIEEWRYGF |
| | QDGTTIVFKN |
| 251 | GCKTSEIAYV KGVKEGLELR YNEQEIVAEE VSWRNDFLHG |
| | ERKIYAGGIQ |
| 301 | KHEWYYRGRS VSKAKFERLN AAG* |

A predicted signal peptide is highlighted.

The cp6890 nucleotide sequence <SEQ ID 132> is:

| | |
|---|---|
| 1 | ATGAAACAAT TACTTTTCTG TGTTTGCGTA TTTGCTATGT |
| | CATGTTCTGC |
| 51 | TTACGCATCC CCACGACGAC AAGATCCTTC TGTTATGAAG |
| | GAAACATTCC |
| 101 | GAAATAATTA TGGCATTATT GTTTCCGGTC AAGAATGGGT |
| | AAAGCGTGGT |
| 151 | TCTGACGGCA CCATCACCAA AGTACTCAAA AATGGAGCTA |
| | CCCTGCATGA |
| 201 | AGTTTATTCT GGAGGCCTCC TTCATGGGGA AATTACCTTA |
| | ACGTTTCCCC |
| 251 | ATACCACAGC ATTGGACGTT GTTCAAATCT ATGATCAAGG |
| | TAGACTCGTT |
| 301 | TCTCGCAAAA CCTTTTTTGT GAACGGTCTT CCATCTCAAG |
| | AAGAGCTGTT |
| 351 | CAATGAAGAT GGCACGTTTG TCCTCACACG ATGGCCGGAC |
| | AACAACGACA |
| 401 | GTGATACCAT CACAAAGCCT TACTTCATAG AAACGACATA |
| | TCAAGGGCAT |
| 451 | GTCATAGAAG GAAGTTATAC TTCCTTTAAT GGGAAATACT |
| | CCTCATCCAT |
| 501 | CCACAATGGA GAGGGAGTTC GTTCTGTGTT CTCCTCCAAT |
| | AACATCCTTC |
| 551 | TTTCTGAAGA GACCTTCAAT GAAGGTGTCA TGGTGAAATA |
| | TACCACATTC |
| 601 | TATCCGAATC GCGATCCCGA ATCGATTACT CATTATCAAA |
| | ATGGACAGCC |
| 651 | TCACGGCTTA CGGCTAACAT ATCTACAAGG TGGCATCCCC |
| | AATACGATAG |
| 701 | AGGAGTGGCG TTATGGCTTT CAAGACGGAA CGACCATCGT |
| | ATTTAAAAAT |
| 751 | GGTTGTAAGA CATCTGAGAT CGCTTATGTT AAGGGAGTGA |
| | AAGAAGGTTT |
| 801 | AGAACTGCGC TACAATGAAC AGGAAATTGT AGCTGAAGAA |
| | GTTTCTTGGC |
| 851 | GTAATGATTT TCTGCATGGA GAACGTAAGA TCTATGCTGG |
| | AGGAATCCAA |
| 901 | AAGCATGAAT GGTATTACCG CGGGAGATCT GTATCTAAAG |
| | CCAAATTCGA |
| 951 | GCGGCTAAAT GCTGCAGGAT AG |

The PSORT algorithm predicts an outer membrane location (0.940).

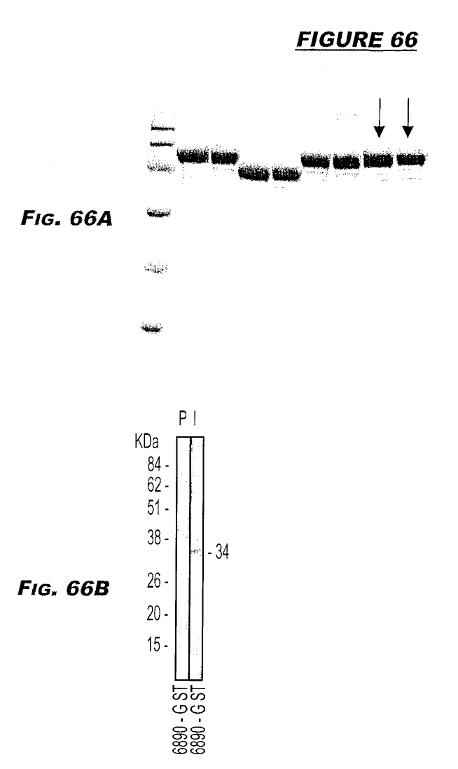

The protein was expressed in E. coli and purified as a GST-fusion product, as shown in FIG. 66A. The recombinant protein was used to immunise mice, whose sera were used in a Western blot (FIG. 66B) and for FACS analysis. A his-tagged protein was also expressed.

These experiments show that cp6890 is a surface-exposed and immunoaccessible protein, and that it is a useful immunogen. These properties are not evident from the sequence alone.

### Example 67

The following C. pneumoniae protein (PID 6172323) was expressed <SEQ ID 133; cp0018>:

| | |
|---|---|
| 1 | MKTSVSMLLA LLCSGASSIV LHAATTPLNP EDGFIGEGNT |
| | NTFSPKSTTD |
| 51 | AGGTTYSLTG EVLYIDPGKG GSITGTCFVE TAGDLTFLGN |
| | GNTLKFLSVD |
| 101 | AGANIAVAHV QGSKNLSFTD FLSLVITESP KSAVTTGKGS |
| | LVSLGAVQLQ |
| 151 | DINTLVLTSN ASVEDGGVIK GNSCLIQGIK NSAIFGQNTS |
| | SKKGGAISTT |
| 201 | QGLTIENNLG TLKFNENKAV TSGGALDLGA ASTETANHEL |
| | IFSQNKTSGN |
| 251 | AANGGAINCS GDLTFTDNTS LLLQENSTMQ DGGALCSTGT |
| | ISITGSDSIN |
| 301 | VIGNTSGQKG GAISAASLKI LGGQGGALFS NNVVTHATPL |
| | GGAIFINTGG |
| 351 | SLQLFTQGGD IVFEGNQVTT TAPNATTKRN VIHLESTAKW |
| | TGLAASQGNA |
| 401 | IYFYDPITTN DTGASDNLRI NEVSANQKLS GSIVFSGERL |
| | STAEAIAENL |
| 451 | TSRINQPVTL VEGSIVLKQG VTLITQGFSQ EPESTLLLDL |
| | GTSL* |

A predicted signal peptide is highlighted.

The cp0018 nucleotide sequence <SEQ ID 134> is:

| | |
|---|---|
| 1 | ATGAAGACTT CAGTTTCTAT GTTGTTGGCC CTGCTTTGCT |
| | CGGGGGCTAG |
| 51 | CTCTATTGTA CTCCATGCCG CAACCACTCC ACTAAATCCT |
| | GAAGATGGGT |
| 101 | TTATTGGGGA GGGCAATACA AATACTTTTT CTCCGAAATC |
| | TACAACGGAT |
| 151 | GCTGCAGGAA CTACCTACTC TCTCACAGGA GAGGTTCTGT |
| | ATATAGATCC |
| 201 | GGGGAAAGGT GGTTCAATTA CAGGAACTTG CTTTGTAGAA |
| | ACTGCTGGCG |
| 251 | ATCTTACATT TTTAGGTAAT GGAAATACCC TAAAGTTCCT |
| | GTCGGTAGAT |
| 301 | GCAGGTGCTA ATATCGCGGT TGCTCATGTA CAAGGAAGTA |
| | AGAATTTAAG |
| 351 | CTTCACAGAT TTCCTTTCTC TGGTGATCAC AGAATCTCCA |
| | AAATCCGCTG |
| 401 | TTACTACAGG AAAAGGTAGC CTAGTCAGTT TAGGTGCAGT |
| | CCAACTGCAA |
| 451 | GATATAAACA CTCTAGTTCT TACAAGCAAT GCCTCTGTCG |
| | AAGATGGTGG |
| 501 | CGTGATTAAA GGAAACTCCT GCTTGATTCA GGGAATCAAA |
| | AATAGTGCGA |
| 551 | TTTTTGGACA AAATACATCT TCGAAAAAAG GAGGGGCGAT |
| | CTCCACGACT |
| 601 | CAAGGACTTA CCATAGAGAA TAACTTAGGG ACGCTAAAGT |
| | TCAATGAAAA |
| 651 | CAAAGCAGTG ACCTCAGGAG GCGCCTTAGA TTTAGGAGCC |
| | GCGTCTACAT |
| 701 | TCACTGCGAA CCATGAGTTG ATATTTTCAC AAAATAAGAC |
| | TTCTGGGAAT |
| 751 | GCTGCAAATG GCGGAGCCAT AAATTGCTCA GGGGACCTTA |
| | CATTTACTGA |
| 801 | TAACACTTCT TTGTTACTTC AAGAAAATAG CACAATGCAG |
| | GATGGTGGAG |
| 851 | CTTTGTGTAG CACAGGAACC ATAAGCATTA CCGGTAGTGA |
| | TTCTATCAAT |
| 901 | GTGATAGGAA ATACTTCAGG ACAAAAAGGA GGAGCGATTT |
| | CTGCAGCTTC |
| 951 | TCTCAAGATT TTGGGAGGGC AGGGAGGCGC TCTCTTTTCT |
| | AATAACGTAG |
| 1001 | TGACTCATGC CACCCCTCTA GGAGGTGCCA TTTTTATCAA |
| | CACAGGAGGA |
| 1051 | TCCTTGCAGC TCTTCACTCA AGGAGGGGAT ATCGTATTCG |
| | AGGGGAATCA |
| 1101 | GGTCACTACA ACAGCTCCAA ATGCTACCAC TAAGAGAAAT |
| | GTAATTCACC |
| 1151 | TCGAGAGCAC CGCGAAGTGG ACGGGACTTG CTGCAAGTCA |
| | AGGTAACGCT |
| 1201 | ATCTATTTCT ATGATCCCAT TACCACCAAC GATACGGGAG |
| | CAAGCGATAA |
| 1251 | CTTACGTATC AATGAGGTCA GTGCAAATCA AAAGCTCTCG |
| | GGATCTATAG |
| 1301 | TATTTTCTGG AGAGAGATTG TCGACAGCAG AAGCTATAGC |
| | TGAAAATCTT |
| 1351 | ACTTCGAGGA TCAACCAGCC TGTCACTTTA GTAGAGGGGA |
| | GCTTAGTACT |
| 1401 | TAAACAGGGA GTGACCTTGA TCACACAAGG ATTCTCGCAG |
| | GAGCCAGAAT |
| 1451 | CCACGCTTCT TTTGGATCTG GGGACCTCAT TATAA |

The PSORT algorithm predicts outer membrane (0.935).

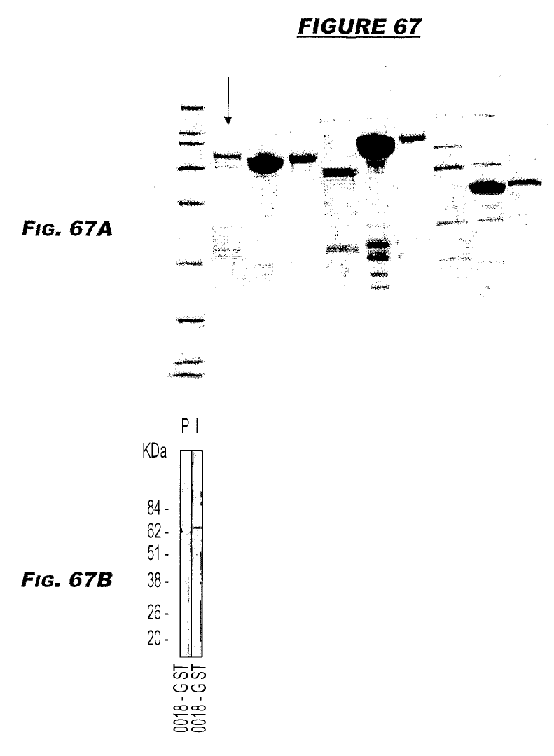

The protein was expressed in E. coli and purified as a GST-fusion product (FIG. 67A). The recombinant protein was used to immunise mice, whose sera were used in a Western blot (FIG. 67B) and for FACS analysis.

These experiments show that cp0018 is a surface-exposed and immunoaccessible protein, and that it is a useful immunogen. These properties are not evident from the sequence alone.

### Example 68

The following C. pneumoniae protein (PID 4376262) was expressed <SEQ ID 135; cp6262>:

| | |
|---|---|
| 1 | MRKLRILAIV LIALSIILIA GGVVLLTVAI PGLSSVISSP |
| | AGMGACALGC |
| 51 | VMLALGIDVL LKKREVPIVL ASVTTTPGTG SPRSGISISG |
| | ADSTIRSLPT |
| 101 | YLLDEGHPQS MRKLRILAIV LIVFSIILIA SGVVLLTVAI |
| | PGLSSVISSP |
| 151 | AGMGACALGC VMLALGIDVL LKKREVPIVL ASVTTTPGTG |
| | SPRSGISISG |
| 201 | ADSTIRSLPT YPLDEGHPQS MRKLRILAIV LIVFSIILIA |
| | SGVVLLTVAI |
| 251 | PGLSSIISSP AEMGACALGC VMLALGIDVL LKKREVPIVV |
| | PAPIPEEVVI |
| 301 | DDIDEESIRL QQEAEAALAR LPEEMSAFEG YIKVVESHLE |
| | NMKSLPYDGH |
| 351 | GLEEKTKHQI RVVRSSLKAM VPEFLDIRRI FEEEEFFFLS |
| | ARKRLIDLAT |
| 401 | TLVERKILTE QLERNNLRKA FSYLYQDSIF KKIIDNFEKL |
| | AWKFMILSKS |
| 451 | ICRFTIIFEN HEHGVAKSLL HKNAVLLEKV IYRSLQKSYR |
| | DIGMSSAKMK |
| 501 | ILHGNPFFSL EDNKKTIMKE HAEMLESLSS YRKVFLALSD |
| | ENVVDTPSDP |
| 551 | KKWDLSGIPC RDALSEISRD EQWQKKAHLK HQESLYTQAR |
| | DRLTDQSSKE |
| 601 | NQKELEKAEQ EYISSWERVK KFEIERVQER IRAIQKLYPN |
| | ILEREEETIG |
| 651 | QETVTPTVQG TTASSDLTDI LGRIEVSSRE DNQNQESCVK |
| | VLRSHEVEMS |
| 701 | WEVKQEYGPK KKEFQDQMGS LERFFTEHIE ELEVLQKDYS |
| | KHLSYFKKVN |
| 751 | NKKEVQYAKF RLKVLESDLE GILAQTESAE SLLTQEELPI |
| | LATRGALEKA |
| 801 | VFKGSLCCAL ASKAKPYFEE DPRFQDSDTQ LRALTLRLQE |
| | AKASLEEEIK |
| 851 | RFSNLENDIA EERRILKESK QTFERAGLGV LREIAVESTY |
| | DLRSLTNTWE |
| 901 | GTPESEKVYF SMYLNYYNEE KRRAKTRLVE MTQRYRDFKM |
| | ALEAMQFNEE |
| 951 | ALLQEELSIQ APSE* |

A predicted signal peptide is highlighted.

The cp6262 nucleotide sequence <SEQ ID 136> is:

| | |
|---|---|
| 1 | ATGAGGAAAC TTCGTATTCT TGCGATCGTT CTCATAGCTT |
| | TGAGCATTAT |
| 51 | TTTGATTGCA GGTGGTGTGG TATTGCTTAC TGTAGCGATC |
| | CCTGGATTAA |
| 101 | GTTCAGTCAT TTCTTCCCCG GCAGGGATGG GTGCCTGTGC |
| | TTTGGGATGT |
| 151 | GTGATGCTTG CTTTAGGGAT CGATGTTCTT CTGAAGAAAC |
| | GAGAAGTCCC |
| 201 | TATAGTTCTC GCATCTGTAA CTACGACACC AGGAACTGGC |
| | AGCCCTAGAA |
| 251 | GTGGTATTTC TATTTCAGGA GCTGATAGCA CCATACGTTC |
| | TCTTCCTACG |
| 301 | TATCTCTTGG ACGAGGGACA TCCACAATCC ATGAGGAAAC |
| | TTCGTATTCT |
| 351 | TGCGATCGTT CTCATAGTTT TTAGCATTAT TTTGATTGCA |
| | AGTGGTGTGG |
| 401 | TATTGCTTAC TGTAGCGATC CCTGGATTAA GTTCAGTCAT |
| | TTCTTCCCCG |
| 451 | GCAGGGATGG GTGCCTGTGC TTTGGGATGT GTGATGCTTG |
| | CTTTAGGGAT |
| 501 | CGATGTTCTT CTGAAGAAAC GAGAAGTCCC TATAGTTCTC |
| | GCATCTGTAA |
| 551 | CTACGACACC AGGAACTGGC AGCCCTAGAA GTGGTATTTC |
| | TATTTCAGGA |
| 601 | GCTGATAGCA CCATACGTTC TCTTCCTACG TATCCCTTGG |
| | ACGAGGGACA |
| 651 | TCCACAATCC ATGAGGAAAC TTCGTATTCT TGCGATCGTT |
| | CTCATAGTTT |
| 701 | TTAGCATTAT TTTGATTGCA AGTGGTGTGG TATTGCTTAC |
| | TGTAGCGATC |
| 751 | CCTGGATTAA GCTCGATCAT TTCTTCCCCA GCGGAGATGG |
| | GTGCTTGTGC |
| 801 | TTTGGGATGT GTGATGCTTG CTTTGGGGAT CGACGTTCTT |
| | CTGAAGAAAC |
| 851 | GAGAAGTCCC TATAGTAGTT CCCGCACCTA TTCCTGAAGA |
| | AGTCGTCATA |
| 901 | GATGATATAG ATGAAGAGAG TATACGGCTG CAGCAGGAAG |
| | CTGAAGCCGC |
| 951 | TTTAGCAAGA CTTCCTGAGG AGATGAGTGC ATTTGAAGGT |
| | TACATAAAAG |
| 1001 | TTGTCGAGAG TCATTTGGAG AACATGAAAA GCCTGCCTTA |
| | TGATGGTCAT |
| 1051 | GGGCTAGAAG AGAAAACGAA ACATCAGATA AGAGTCGTCA |
| | GATCTTCTTT |
| 1101 | GAAGGCTATG GTTCCAGAAT TTTTAGATAT CAGAAGAATT |
| | TTTGAAGAAG |
| 1151 | AAGAGTTCTT TTTTCTCTCA GCTCGCAAAC GACTTATAGA |
| | TTTAGCTACT |
| 1201 | ACTTTAGTAG AGAGAAAAAT TTTAACAGAG CAACTTGAGC |
| | GCAATAATTT |
| 1251 | AAGGAAAGCG TTTTCTTATT TATATCAGGA CTCAATTTTT |
| | AAAAAAATTA |
| 1301 | TTGATAACTT CGAGAAGTTA GCATGGAAAT TTATGATTTT |
| | GAGTAAATCA |
| 1351 | ATTTGTCGAT TTACAATTAT TTTTGAAAAT CATGAACATG |
| | GTGTAGCAAA |
| 1401 | GAGCCTGTTA CACAAGAATG CAGTGTTACT GGAGAAGGTA |
| | ATCTATAGGA |
| 1451 | GTTTGCAAAA AAGCTATAGA GATATAGGCA TGTCATCTGC |
| | AAAGATGAAA |
| 1501 | ATCTTGCACG GCAACCCTTT TTTCTCTTTG GAAGATAATA |
| | AAAAGACGAT |
| 1551 | AATGAAAGAA CACGCAGAGA TGCTTGAAAG TCTCAGTAGC |
| | TATAGGAAGG |
| 1601 | TATTTTTAGC TCTATCTGAT GAGAACGTTG TAGATACACC |
| | TAGCGATCCA |
| 1651 | AAGAAATGGG ATTTGTCAGG AATCCCCTGT AGGGACGCGT |
| | TGTCTGAGAT |
| 1701 | TTCTCGTGAT GAACAGTGGC AGAAGAAAGC ACATCTAAAG |
| | CATCAAGAGT |
| 1751 | CCCTCTATAC GCAAGCTAGG GATCGTTTAA CAGACCAGAG |
| | CTCTAAAGAA |
| 1801 | AATCAGAAAG AGTTAGAGAA AGCTGAACAA GAGTACATAT |
| | CTTCTTGGGA |
| 1851 | ACGGGTTAAA AAATTTGAGA TTGAGAGAGT ACAGGAGAGG |
| | ATACGGGCAA |
| 1901 | TTCAAAAGCT TTATCCTAAT ATCCTCGAGA GAGAAGAAGA |
| | AACCACAGGT |
| 1951 | CAGGAGACTG TGACTCCAAC TGTTCAAGGG ACGACGGCTT |
| | CATCCGATTT |
| 2001 | AACAGATATT TTAGGAAGAA TAGAGGTCTC CAGTAGGGAG |
| | GATAATCAGA |
| 2051 | ATCAAGAGTC TTGTGTAAAA GTCTTAAGAA GTCATGAGGT |
| | AGAAATGAGC |
| 2101 | TGGGAAGTCA AACAAGAGTA TGGCCCTAAG AAAAAAGAAT |
| | TTCAGGATCA |
| 2151 | AATGGGTTCT TTAGAGAGGT TTTTTACAGA GCATATTGAA |
| | GAGTTAGAAG |
| 2201 | TATTACAGAA GGACTACTCT AAACACTTGT CTTATTTTAA |
| | AAAAGTAAAC |
| 2251 | AATAAGAAAG AGGTTCAATA TGCGAAGTTT AGGTTGAAGG |
| | TTTTAGAGTC |
| 2301 | AGATTTAGAA GGGATTCTAG CTCAGACTGA GAGTGCTGAG |
| | AGTCTGTTAA |
| 2351 | CTCAAGAAGA ACTTCCGATT CTTGCAACTC GGGGAGCCTT |
| | AGAGAAAGCT |
| 2401 | GTTTTCAAAG GGAGTCTATG TTGCGCGCTA GCAAGCAAAG |
| | CAAAACCCTA |
| 2451 | TTTTGAAGAG GATCCCAGAT TCCAAGATTC TGATACGCAA |
| | TTGCGAGCTC |
| 2501 | TGACTCTAAG GTTACAGGAG GCTAAGGCAA GCCTGGAAGA |
| | AGAGATAAAG |
| 2551 | AGATTTTCAA ATCTTGAGAA CGATATTGCA GAGGAAAGAC |
| | GCCTTCTTAA |
| 2601 | AGAGAGCAAG CAGACGTTCG AAAGAGCAGG TTTAGGGGTT |
| | CTCCGAGAAA |
| 2651 | TTGCAGTCGA GTCTACTTAT GATTTGCGTT CCTTAACAAA |
| | TACATGGGAA |
| 2701 | GGGACCCCAG AGAGTGAGAA GGTCTATTTT AGCATGTATC |
| | TTAATTATTA |
| 2751 | CAACGAAGAG AAACGTAGGG CTAAAACAAG ATTGGTTGAA |
| | ATGACACAGA |
| 2801 | GGTATAGAGA TTTTAAAATG GCCTTGGAAG CTATGCAGTT |
| | TAATGAAGAA |
| 2851 | GCCCTTTTGC AAGAGGAACT CTCTATTCAA GCTCCCAGTG |
| | AATAA |

The PSORT algorithm predicts inner membrane (0.660).

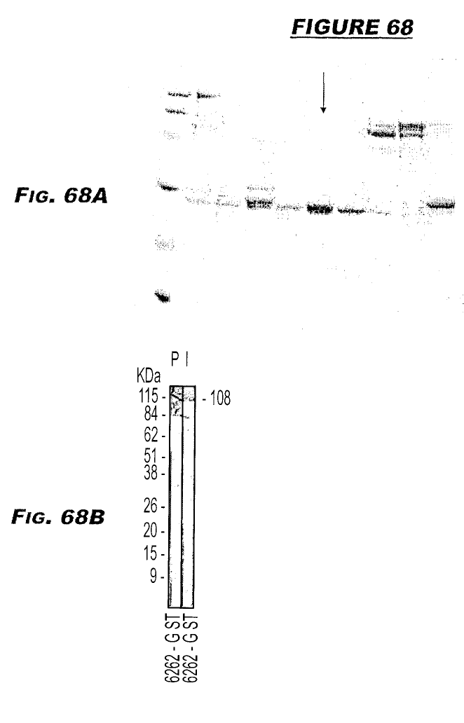

The protein was expressed in E. coli and purified as a GST-fusion product (FIG. 68A). The recombinant protein was used to immunise mice, whose sera were used in a Western blot (FIG. 68B) and for FACS analysis.

These experiments show that cp6262 is a surface-exposed and immunoaccessible protein, and that it is a useful immunogen. These properties are not evident from the sequence alone.

### Example 69

The following C. pneumoniae protein (PID 4376269) was expressed <SEQ ID 137; cp6269>:

| | |
|---|---|
| 1 | MYQENLRLLE RLLYNSVQKS YADRLFSYEK TKMVHDTPLI |
| | PWEEDKEKCA |
| 51 | EAEKAFLEQQ KILLDYGKSI FWLNENDEIN LNDPWSWGLN |
| | TVRTRKVFQE |
| 101 | VDDSERWNHK VLIQKLEDDY EKLLEESSKE STEANKKLLS |
| | DLVDRLEDAK |
| 151 | TKFFLKKQEE VETRVKDLRA RYGGTVDPKQ DTEAKKKVEL |
| | EASLETFLDS |
| 201 | IESELVQCLE DQDIYWKEQD VKDLARTQEL EEQDIEAKRE |
| | EAAEDLRSLN |
| 251 | ERLKKSKTML DRAKWHIENA EDSITWWTSQ IEMKDMKARL |
| | KILKEDITSV |
| 301 | LPEIDEIETC LSLEELPLLT TRELLTKSYL KFKICSETLL |
| | KMTSVFENNI |
| 351 | YVQEYEVQLQ NLGFKLQGIS QRFGKKQDDF ANLEEQVALQ |
| | KKRLRELTQN |
| 401 | FEIQGFNFMK EDFKAAAKDL YIRSTAEQKM NFDVPCMELF |
| | RRYHEEVNKP |
| 451 | LLELMYNCAD SYRDAKKKLC SLRLDEKELL QKEIKKEEFY |
| | QKKQQRHADR |
| 501 | SRHTTYQKLR IAEELALELK KKI* |

The cp6269 nucleotide sequence <SEQ ID 138> is:

| | |
|---|---|
| 1 | ATGTACCAGG AGAATCTAAG ATTGTTGGAA AGGCTTCTTT |
| | ATAATAGTGT |
| 51 | TCAAAAGAGC TATGCGGATC GGCTGTTTTC CTATGAAAAG |
| | ACAAAGATGG |
| 101 | TGCACGATAC TCCGCTGATT CCTTGGGAAG AGGATAAGGA |
| | AAAATGTGCT |
| 151 | GAAGCTGAGA AAGCTTTCTT AGAGCAACAG AAGATTCTCC |
| | TAGATTATGG |
| 201 | AAAATCTATC TTTTGGCTGA ATGAGAACGA TGAGATCAAT |
| | TTAAACGATC |
| 251 | CTTGGAGTTG GGGTCTTAAT ACGGTGAGGA CTAGGAAAGT |
| | ATTCCAAGAG |
| 301 | GTTGACGACA GTGAACGTTG GAATCATAAG GTACTCATTC |
| | AAAAACTCGA |
| 351 | GGACGATTAT GAGAAACTTC TAGAGGAAAG TTCAAAAGAG |
| | TCTACTGAAG |
| 401 | CAAATAAGAA GCTTTTATCT GACTTAGTAG ATCGTCTTGA |
| | AGATGCTAAG |
| 451 | ACAAAATTTT TCCTGAAGAA ACAGGAGGAG GTGGAGACTC |
| | GCGTTAAGGA |
| 501 | TCTTAGACCT CGATATGGAG GCACAGTAGA TCCTAAGCAG |
| | GATACGGAAG |
| 551 | CTAAGAAGAA AGTCGAATTG GAGGCTAGCT TAGAAACCTT |
| | TTTAGATTCC |
| 601 | ATCGAATCAG AGCTAGTACA GTGTTTAGAA GATCAAGATA |
| | TATATTGGAA |
| 651 | AGAACAGGAT GTCAAAGATC TAGCACGTAC GCAAGAGCTC |
| | GAGGAACAAG |
| 701 | ATATTGAAGC GAAGAGGGAA GAAGCTGCCG AAGACCTAAG |
| | AAGTCTTAAT |
| 751 | GAGCGTTTAA AGAAGTCAAA AACTATGTTA GATAGGGCTA |
| | AATGGCATAT |
| 801 | TGAAAATGCT GAGGACAGTA TTACCTGGTG GACTAGTCAG |
| | ATAGAAATGA |
| 851 | AGGATATGAA AGCAAGACTG AAGATCTTAA AAGAAGATAT |
| | AACAAGTGTT |
| 901 | CTACCTGAAA TAGATGAGAT TGAAACGTGT TTAAGCTTAG |
| | AGGAGCTTCC |
| 951 | TTTGCTTACG ACCAGGGAAC TCTTAACTAA GTCCTACCTA |
| | AAGTTTAAGA |
| 1001 | TTTGTTCGGA AACACTATTA AAAATGACTT CTGTGTTTGA |
| | GAACAATATC |
| 1051 | TATGTTCAGG AGTACGAGGT TCAGCTGCAA AATCTAGGGT |
| | TTAAGTTACA |
| 1101 | AGGTATATCT CAGAGATTCG GAAAGAAACA AGACGATTTT |
| | GCGAATCTAG |
| 1151 | AGGAACAGGT TGCTTTGCAA AAGAAACGAC TCAGAGAGCT |
| | CACTCAGAAT |
| 1201 | TTTGAAATAC AAGGATTCAA TTTCATGAAA GAAGATTTTA |
| | AGGCAGCCGC |
| 1251 | TAAAGATCTT TATATAAGAA GTACAGCTGA ACAAAAGATG |
| | AACTTTGATG |
| 1301 | TGCCTTGCAT GGAGCTCTTC CGTAGGTATC ATGAGGAGGT |
| | CAACAAGCCG |
| 1351 | CTTCTTGAGT TGATGTACAA TTGTGCAGAC AGTTATAGAG |
| | ATGCTAAGAA |
| 1401 | AAAGCTTTGC TCTCTACGTC TTGATGAAAA AGAGTTATTA |
| | CAAAAAGAAA |
| 1451 | TCAAGAAAGA GGAATTTTAT CAAAAGAAAC AACAAAGGCA |
| | TGCAGATAGA |
| 1501 | TCACGTCATA CTACGTATCA AAAGCTACGA ATTGCTGAAG |
| | AGCTTGCTCT |
| 1551 | TGAGCTGAAG AAGAAAATCT AA |

The PSORT algorithm predicts cytoplasmic location (0.412).

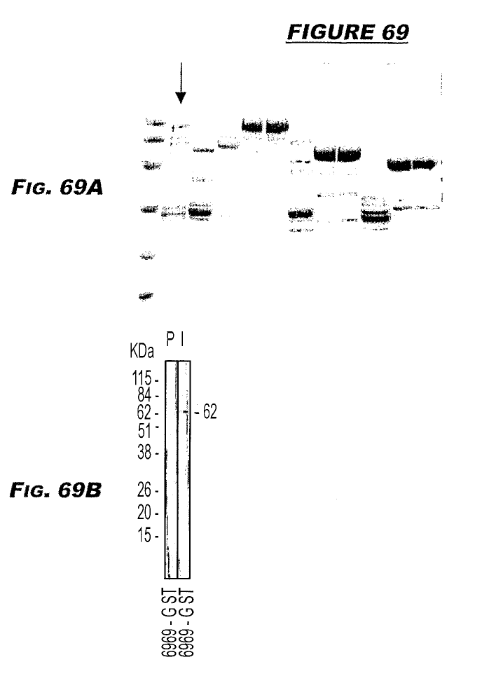

The protein was expressed in E. coli and purified as a GST-fusion product (FIG. 69A). The recombinant protein was used to immunise mice, whose sera were used in a Western blot (FIG. 69B) and for FACS analysis.

These experiments show that cp6269 is a surface-exposed and immunoaccessible protein, and that it is a useful immunogen. These properties are not evident from the sequence alone.

### Example 70

The following C. pneumoniae protein (PID 4376270) was expressed <SEQ ID 139; cp6270>:

| | |
|---|---|
| 1 | MKIPLRFLLI SLVPTLSMSN LLGAATTEEL SASNSFDGTT |
| | STTSFSSKTS |
| 51 | SATDGTNYVF KDSVVIENVP KTGETQSTSC FKNDAAAGDL |
| | NFLGGGFSFT |
| 101 | FSNIDATTAS GAAIGSEAAN KTVTLSGFSA LSFLKSPAST |
| | VTNGLGAINV |
| 151 | KGNLSLLDND KVLIQDNFST GDGGAINCAG SLKIANNKSL |
| | SFIGNSSSTR |
| 201 | GGAIHTKNLT LSSGGETLFQ GNTAPTAAGK GGAIAIADSG |
| | TLSISGDSGD |
| 251 | IIFEGNTIGA TGTVSHSAID LGTSADITAL RAAQGHTIYF |
| | YDPITVTGST |
| 301 | SVADALNINS PDTGDNKEYT GTIVFSGEKL TEAEAKDEKN |
| | RTSKLLQNVA |
| 351 | FKNGTVVLKG DVVLSANGFS QDANSKLIMD LGTSLVANTE |
| | SIELTNLEIN |
| 401 | IDSLRNGKKI KLSAATAGKD IRIDRPVVLA ISDESFYQNG |
| | FLNEDHSYDG |
| 451 | ILELDAGKDI VISADSRSIT AVQSPYGYQG KWTINWSTDD |
| | KKATVSWAKQ |
| 501 | SFNPTAEQEA PLVPNLLWGS FIDVRSFQNF IELGTEGAPY |
| | EKRFWVAGIS |
| 551 | NVLHRSGREN QRKFRHVSGG AVVGASTRMP GGDTLSLGFA |
| | QLFARDKDYF |
| 601 | MNTNFAKTYA GSLRLQHDAS LYSVVSILLG EGGLREILLP |
| | YVSKTLPCSF |
| 651 | YGQLSYGHTD HRMKTESLPP PPPTLSTDHT SWGGYVWAGE |
| | LGTRVAVENT |
| 701 | SGRGFFQEYT PFVKVQAVYA RQDSFVELGA ISRDFSDSHL |
| | YNLAIPLGIK |
| 751 | LEKRFAEQYY HVVAMYSPDV CRSNPKCTTT LLSNQGSWKT |
| | KGSNLARQAG |
| 801 | IVQASGFRSL GAAAELFGNF GFEWRGSSRS YNVDAGSKIK F* |

A predicted signal peptide is highlighted.

The cp6270 nucleotide sequence <SEQ ID 140> is:

| | |
|---|---|
| 1 | ATGAAGATTC CACTCCGCTT TTTATTGATA TCATTAGTAC |
| | CTACGCTTTC |
| 51 | TATGTCGAAT TTATTAGGAG CTGCTACTAC CGAAGAGTTA |
| | TCGGCTAGCA |
| 101 | ATAGCTTCGA TGGAACTACA TCAACAACAA GCTTTTCTAG |
| | TAAAACATCA |
| 151 | TCGGCTACAG ATGGCACCAA TTATGTTTTT AAAGATTCTG |
| | TAGTTATAGA |
| 201 | AAATGTACCC AAAACAGGGG AAACTCAGTC TACTAGTTGT |
| | TTTAAAAATG |
| 251 | ACGCTGCAGC TGGAGATCTA AATTTCTTAG GAGGGGGATT |
| | TTCTTTCACA |
| 301 | TTTAGCAATA TCGATGCAAC CACGGCTTCT GGAGCTGCTA |
| | TTGGAAGTGA |
| 351 | AGCAGCTAAT AAGACAGTCA CGTTATCAGG ATTTTCGGCA |
| | CTTTCTTTTC |
| 401 | TTAAATCCCC AGCAAGTACA GTGACTAATG GATTGGGAGC |
| | TATCAATGTT |
| 451 | AAAGGGAATT TAAGCCTATT GGATAATGAT AAGGTATTGA |
| | TTCAGGACAA |
| 501 | TTTCTCAACA GGAGATGGCG GAGCAATTAA TTGTGCAGGC |
| | TCCTTGAAGA |
| 551 | TCGCAAACAA TAAGTCCCTT TCTTTTATTG GAAATAGTTC |
| | TTCAACACGT |
| 601 | GGCGGAGCGA TTCATACCAA AAACCTCACA CTATCTTCTG |
| | GTGGGGAAAC |
| 651 | TCTATTTCAG GGGAATACAG CGCCTACGGC TGCTGGTAAA |
| | GGAGGTGCTA |
| 701 | TCGCGATTGC AGACTCTGGC ACCCTATCCA TTTCTGGAGA |
| | CAGTGGCGAC |
| 751 | ATTATCTTTG AAGGCAATAC GATAGGAGCT ACAGGAACCG |
| | TCTCTCATAG |
| 801 | TGCTATTGAT TTAGGAACTA GCGCTAAGAT AACTGCGTTA |
| | CGTGCTGCGC |
| 851 | AAGGACATAC GATATACTTT TATGATCCGA TTACTGTAAC |
| | AGGATCGACA |
| 901 | TCTGTTGCTG ATGCTCTCAA TATTAATAGC CCTGATACTG |
| | GAGATAACAA |
| 951 | AGAGTATACG GGAACCATAG TCTTTTCTGG AGAGAAGCTC |
| | ACGGAGGCAG |
| 1001 | AAGCTAAAGA TGAGAAGAAC CGCACTTCTA AATTACTTCA |
| | AAATGTTGCT |
| 1051 | TTTAAAAATG GGACTGTAGT TTTAAAAGGT GATGTCGTTT |
| | TAAGTGCGAA |
| 1101 | CGGTTTCTCT CAGGATGCAA ACTCTAAGTT GATTATGGAT |
| | TTAGGGACGT |
| 1151 | CGTTGGTTGC AAACACCGAA AGTATCGAGT TAACGAATTT |
| | GGAAATTAAT |
| 1201 | ATAGACTCTC TCAGGAACGG GAAAAAGATA AAACTCAGTG |
| | CTGCCACAGC |
| 1251 | TCAGAAAGAT ATTCGTATAG ATCGTCCTGT TGTACTGGCA |
| | ATTAGCGATG |
| 1301 | AGAGTTTTTA TCAAAATGGC TTTTTGAATG AGGACCATTC |
| | CTATGATGGG |
| 1351 | ATTCTTGAGT TAGATGCTGG GAAAGACATC GTGATTTCTG |
| | CAGATTCTCG |
| 1401 | CAGTATAGAT GCTGTACAAT CTCCGTATGG CTATCAGGGA |
| | AAGTGGACGA |
| 1451 | TCAATTGGTC TACTGATGAT AAGAAAGCTA CGGTTTCTTG |
| | GGCGAAGCAG |
| 1501 | AGTTTTAATC CCACTGCTGA GCAGGAGGCT CCGTTAGTTC |
| | CTAATCTTCT |
| 1551 | TTGGGGTTCT TTTATAGATG TTCGTTCCTT CCAGAATTTT |
| | ATAGAGCTAG |
| 1601 | GTACTGAAGG TGCTCCTTAC GAAAAGAGAT TTTGGGTTGC |
| | AGGCATTTCC |
| 1651 | AATGTTTTGC ATAGGAGCGG TCGTGAAAAT CAAAGGAAAT |
| | TCCGTCATGT |
| 1701 | GAGTGGAGGT GCTGTAGTAG GTGCTAGCAC GAGGATGCCG |
| | GGTGGTGATA |
| 1751 | CCTTGTCTCT GGGTTTTGCT CAGCTCTTTG CGCGTGACAA |
| | AGACTACTTT |
| 1801 | ATGAATACCA ATTTCGCAAA GACCTACGCA GGATCTTTAC |
| | GTTTGCAGCA |
| 1851 | CGATGCTTCC CTATACTCTG TGGTGAGTAT CCTTTTAGGA |
| | GAGGGAGGAC |
| 1901 | TCCGCGAGAT CCTGTTGCCT TATGTTTCCA AGACTCTGCC |
| | GTGCTCTTTC |
| 1951 | TATGGGCAGC TTAGCTACGG CCATACGGAT CATCGCATGA |
| | AGACCGAGTC |
| 2001 | TCTACCCCCC CCCCCCCCGA CGCTCTCGAC GGATCATACT |
| | TCTTGGGGAG |
| 2051 | GATATGTCTG GGCTGGAGAG CTGGGAACTC GAGTTGCTGT |
| | TGAAAATACC |
| 2101 | AGCGGCAGAG GATTTTTCCA AGAGTACACT CCATTTGTAA |
| | AAGTCCAAGC |
| 2151 | TGTTTACGCT CGCCAAGATA GCTTTGTAGA ACTAGGAGCT |
| | ATCAGTCGTG |
| 2201 | ATTTTAGTGA TTCGCATCTT TATAACCTTG CGATTCCTCT |
| | TGGAATCAAG |
| 2251 | TTAGAGAAAC GGTTTGCAGA GCAATATTAT CATGTTGTAG |
| | CGATGTATTC |
| 2301 | TCCAGATGTT TGTCGTAGTA ACCCCAAATG TACGACTACC |
| | CTACTTTCCA |
| 2351 | ACCAAGGGAG TTGGAAGACC AAAGGTTCGA ACTTAGCAAG |
| | ACAGGCTGGT |
| 2401 | ATTGTTCAGG CCTCAGGTTT TCGATCTTTG GGAGCTGCAG |
| | CAGAGCTTTT |
| 2451 | CGGGAACTTT GGCTTTGAAT GGCGGGGATC TTCTCGTAGC |
| | TATAATGTAG |
| 2501 | ATGCGGGTAG CAAAATCAAA TTTTAG |

The PSORT algorithm predicts outer membrane (0.92).

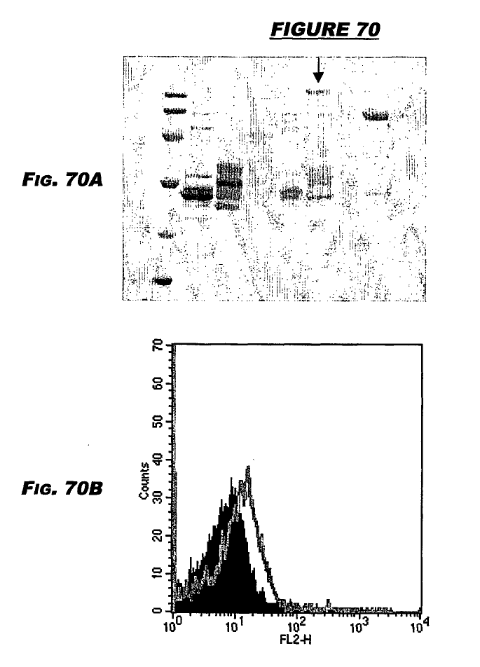

The protein was expressed in E. coli and purified as a GST-fusion product (FIG. 70A). The recombinant protein was used to immunise mice, whose sera were used in a Western blot and for FACS analysis (FIG. 70B).

The cp6270 protein was also identified in the 2D-PAGE experiment (Cpn0013).

These experiments show that cp6270 is a surface-exposed and immunoaccessible protein, and that it is a useful immunogen. These properties are not evident from the sequence alone.

### Example 71

The following C. pneumoniae protein (PID 4376402) was expressed <SEQ ID 141; cp6402>:

| | |
|---|---|
| 1 | MNVADLLSHL ETLLSSKIFQ DYGPNGLQVG DPQTPVKKIA |
| | VAVTADLETI |
| 51 | KQAVAAEANV LIVHHGIFWK GMPYPITGMI HKRIQLLIEH |
| | NIQLIAYHLP |
| 101 | LDAHPTLGNN WRVALDLNWH DLKPFGSSLP YLGVQGSFSP |
| | IDIDSFIDLL |
| 151 | SQYYQAPLKG SALGGPSRVS SAALISGGAY RELSSAATSQ |
| | VDCFITGNFD |
| 201 | EPAWSTALES NINFLAFGHT ATEKVGPKSL AEHLKSEFPI |
| | STTFIDTANP |
| 251 | F* |

The cp6402 nucleotide sequence <SEQ ID 142> is:

| | |
|---|---|
| 1 | ATGAATGTTG CGGATCTCCT TTCTCATCTT GAGACTCTTC |
| | TCTCATCAAA |
| 51 | AATATTTCAG GATTATGGAC CCAACGGACT TCAAGTTGGA |
| | GATCCCCAAA |
| 101 | CTCCGGTAAA GAAAATCGCT GTTGCAGTTA CCGCAGATCT |
| | AGAAACCATA |
| 151 | AAACAAGCTG TTGCGGCCGA AGCAAACGTT CTCATTGTAC |
| | ACCACGGAAT |
| 201 | TTTTTGGAAA GGTATGCCCT ATCCTATTAC CGGCATGATC |
| | CATAAGCGCA |
| 251 | TCCAATTACT AATAGAACAC AATATCCAAC TCATTGCCTA |
| | CCACCTTCCT |
| 301 | TTGGATGCTC ACCCTACCTT AGGAAATAAC TGGAGAGTTG |
| | CCCTGGATCT |
| 351 | AAATTGGCAT GACTTGAAGC CCTTTGGTTC TTCCCTCCCT |
| | TATTTAGGAG |
| 401 | TGCAAGGCTC TTTCTCTCCT ATCGATATAG ATTCTTTCAT |
| | TGACCTGTTA |
| 451 | TCTCAATATT ACCAAGCTCC CCTAAAAGGA TCTGCCTTGG |
| | GCGGCCCCTC |
| 501 | TAGAGTCTCC TCAGCAGCTC TGATCTCAGG AGGAGCTTAT |
| | AGAGAACTCT |
| 551 | CTTCGGCAGC CACGTCCCAA GTCGATTGCT TCATCACAGG |
| | AAATTTTGAT |
| 601 | GAACCTGCAT GGTCGACAGC TCTAGAAAGC AATATCAACT |
| | TCCTAGCATT |
| 651 | TGGACATACA GCCACAGAAA AAGTAGGTCC AAAATCTCTT |
| | GCAGAGCATC |
| 701 | TAAAAAGCGA ATTTCCTATT TCCACAACCT TTATAGATAC |
| | GGCCAACCCC |
| 751 | TTCTAA |

The PSORT algorithm predicts cytoplasmic (0.158).

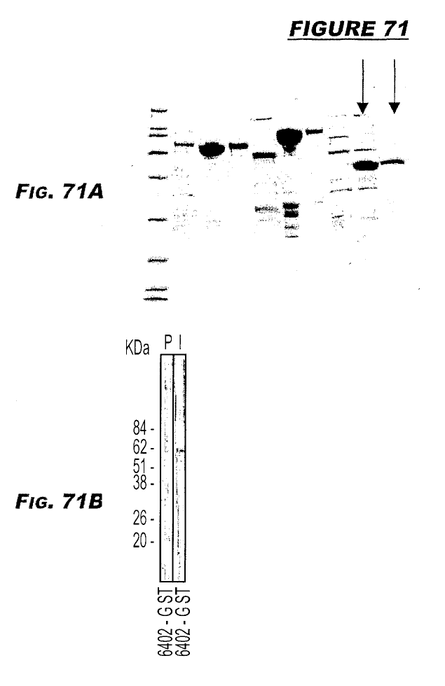

The protein was expressed in E. coli and purified as a GST-fusion product (FIG. 71A). The recombinant protein was used to immunise mice, whose sera were used in a Western blot (FIG. 71B) and for FACS analysis.

These experiments show that cp6402 is a surface-exposed and immunoaccessible protein, and that it is a useful immunogen. These properties are not evident from the sequence alone.

### Example 72

The following C. pneumoniae protein (PID 4376520) was expressed <SEQ ID 143; cp6520>:

| | |
|---|---|
| 1 | MKHYLSFSPS ADFFSKQGAI ETQVLFGERV LVKGSTCYAY |
| | SQLFHNELLW |
| 51 | KPYPGHSFRS TLVPCTPEFH IHPNVSVVSV DAFLDPWGIP |
| | LPFGTLLHVN |
| 101 | SQNTVIFPKD ILNHMNTIWG SGTPQCDPRH LRRLNYNFFA |
| | ELLIKDADLL |
| 151 | LNFPYVWGGR SVHESLEKPG VDCSGFINIL YQAQGYNVPR |
| | NAADQYADCH |
| 201 | WISSFENLPS GGLIFLYPKE EKRISHVMLK QDSSTLIHAS |
| | GGGKKVEYFI |
| 251 | LEQDGKFLDS TYLFFRNNQR GRAFFGIPRK RKAFL* |

The cp6520 nucleotide sequence <SEQ ID 144> is:

| | |
|---|---|
| 1 | ATGAAACACT ACCTATCATT TTCTCCTTCT GCTGATTTTT |
| | TCTCTAAACA |
| 51 | GGGTGCTATT GAAACTCAAG TCCTTTTTGG AGAGCGCGTC |
| | TTAGTCAAAG |
| 101 | GGAGCACCTG CTATGCATAT TCCCAATTAT TCCACAATGA |
| | GCTGTTATGG |
| 151 | AAGCCCTATC CAGGTCATAG CTTTCGTTCT ACCCTAGTCC |
| | CCTGCACTCC |
| 201 | TGAATTTCAT ATCCATCCAA ATGTTTCTGT GGTTTCTGTG |
| | GATGCATTTT |
| 251 | TAGATCCTTG GGGGATCCCT CTTCCTTTTG GAACTTTACT |
| | CCATGTGAAT |
| 301 | TCTCAAAATA CCGTTATTTT CCCTAAGGAT ATTCTCAATC |
| | ATATGAACAC |
| 351 | CATCTGGGGC TCCGGCACAC CTCAATGCGA TCCTAGACAT |
| | CTACGTCGTC |
| 401 | TAAATTATAA CTTCTTTGCT GAACTTTTAA TTAAAGACGC |
| | AGACCTTTTA |
| 451 | CTGAACTTTC CCTATGTATG GGGAGGACGG TCTGTACACG |
| | AAAGTCTGGA |
| 501 | AAAGCCGGGT GTTGATTGTT CGGGATTTAT CAATATCCTT |
| | TACCAGGCAC |
| 551 | AGGGATACAA CGTCCCTAGA AACGCTGCAG ATCAATATGC |
| | GGATTGTCAT |
| 601 | TGGATCTCTA GCTTTGAGAA CCTTCCTTCT GGTGGGTTAA |
| | TATTTCTTTA |
| 651 | CCCTAAAGAA GAAAAGCGTA TTTCTCATGT TATGTTGAAA |
| | CAGGATAGTT |
| 701 | CCACCCTCAT TCATGCTTCT GGTGGAGGGA AAAAAGTGGA |
| | GTATTTCATT |
| 751 | TTAGAACAAG ATGGGAAGTT TTTAGATTCG ACTTATCTAT |
| | TTTTTAGAAA |
| 801 | TAATCAGAGG GGACGGGCAT TTTTTGGGAT CCCTAGAAAA |
| | AGAAAAGCCT |
| 851 | TTCTGTAA |

The PSORT algorithm predicts cytoplasmic (0.265).

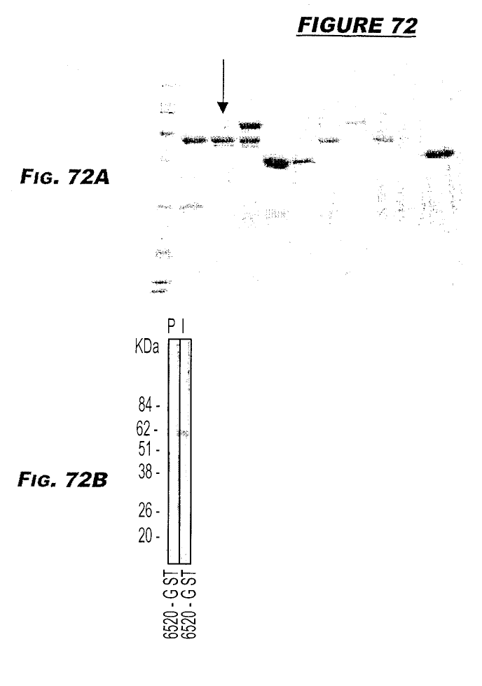

The protein was expressed in E. coli and purified as a GST-fusion product (FIG. 72A). The recombinant protein was used to immunise mice, whose sera were used in a Western blot (FIG. 72B) and for FACS analysis.

These experiments show that cp6520 is a surface-exposed and immunoaccessible protein, and that it is a useful immunogen. These properties are not evident from the sequence alone.

### Example 73

The following C. pneumoniae protein (PID 4376567) was expressed <SEQ ID 145; cp6567>:

| | |
|---|---|
| 1 | MTSPIPFQSS GDASFLAEQP QQLPSTSESQ LVTQLLTMMK |
| | HTQALSETVL |
| 51 | QQQRDRLPTA SILLQVGGAP TGGAGAPFQP GPADDHHHPI |
| | PPPVVPAQIE |
| 101 | TEITTIRSEL QLMRSTLQQS TKGARTGVLV VTAILMTISL |
| | LAIIIIILAV |
| 151 | LGFTGVLPQV ALLMQGETNL IWAMVSGSII CFIALIGTLG |
| | LILTNKNTPL |
| 201 | PAS* |

The cp6567 nucleotide sequence <SEQ ID 146> is:

| | |
|---|---|
| 1 | ATGACCTCAC CGATCCCCTT TCAGTCTAGT GGCGATGCCT |
| | CTTTCCTTGC |
| 51 | CGAGCAGCCA CAGCAACTCC CGTCTACTTC TGAATCTCAG |
| | CTAGTAACTC |
| 101 | AATTGCTAAC CATGATGAAG CATACTCAAG CATTATCCGA |
| | AACGGTTCTT |
| 151 | CAACAACAAC GCGATCGATT ACCAACCGCA TCTATTATCC |
| | TTCAAGTAGG |
| 201 | AGGAGCTCCT ACAGGAGGAG CGGGTGCGCC TTTTCAACCA |
| | GGACCGGCAG |
| 251 | ATGATCATCA TCATCCCATA CCGCCGCCTG TTGTACCAGC |
| | TCAAATAGAA |
| 301 | ACAGAAATCA CCACTATAAG ATCCGAGTTA CAGCTCATGC |
| | GATCTACTCT |
| 351 | ACAACAAAGC ACAAAAGGAG CTCGTACAGG AGTTCTAGTG |
| | GTTACTGCAA |
| 401 | TCTTAATGAC GATCTCCTTA TTGGCTATTA TTATCATAAT |
| | ACTAGCTGTG |
| 451 | CTTGGATTTA CGGGCGTCTT GCCTCAAGTA GCTTTATTGA |
| | TGCAGGGTGA |
| 501 | AACAAATCTG ATTTGGGCTA TGGTGAGCGG TTCTATTATT |
| | TGCTTTATTG |
| 551 | CGCTAATTGG AACTCTAGG ATTAATTTTAA CAAATAAGAA |
| | CACGCCTCTA |
| 601 | CCGGCTTCTT AA |

The PSORT algorithm predicts inner membrane (0.694).

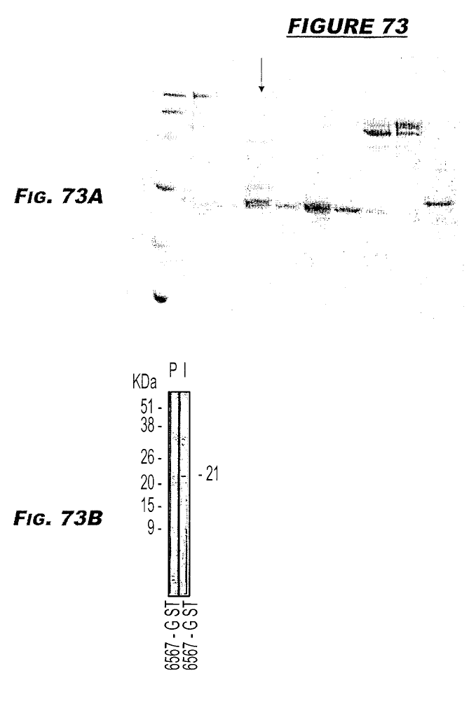

The protein was expressed in E. coli and purified as a GST-fusion product (FIG. 73A). The recombinant protein was used to immunise mice, whose sera were used in a Western blot (FIG. 73B) and for FACS analysis.

These experiments show that cp6567 is a surface-exposed and immunoaccessible protein, and that it is a useful immunogen. These properties are not evident from the sequence alone.

### Example 74

The following C. pneumoniae protein (PID 4376576) was expressed <SEQ ID 147; cp6576>:

| | |
|---|---|
| 1 | MLIMRNKVIL QISILALIQT PLTLFSTEKV KEGHVVVDSI |
| | TIITEGENAS |
| 51 | NKHPLPKLKT RSGALFSQLD FDEDLRILAK EYDSVEPKVE |
| | FSEGKTNIAL |
| 101 | HLIAKPSIRN IHISGNQVVP EHKILKTLQI YRNDLFEREK |
| | FLKGLDDLRT |
| 151 | YYLKRGYFAS SVDYSLEHNQ EKGHIDVLIK INEGPCGKIK |
| | QLTFSGISRS |
| 201 | EKSDIQEFIQ TKQHSTTTSW FTGAGLYHPD IVEQDSLAIT |
| | NYLHNNGYAD |
| 251 | AIVNSHYDLD DKGNILLYMD IDRESRYTLG HVHIQGFEVL |
| | PKRLIEKQSQ |
| 301 | VGPNDLYCPD KIWDGAHKIK QTYAKYGYIN TNVDVLFIPH |
| | ATRPIYDVTY |
| 351 | EVSFGSPYKV GLIKITGNTH TKSDVILHET SLFPGDTFNR |
| | LKLEDTEQRL |
| 401 | RNTGYFQSVS VYTVRSQLDP MGNADQYRDI FVEVKETTTG |
| | NLGLFLGFSS |
| 451 | LDNLFGGIEL SESNFDLFGA RNIFSKGFRC LRGGGEHLFL |
| | KANFGDKVTD |
| 501 | YILKWTKPHF LNTPWILGIE LDKSINRALS KDYAVQTYGG |
| | NVSTTYILNE |
| 551 | HLKYGLFYRG SQTSLHEKRK FLLGPNIDSN KGFVSAAGVN |
| | LNYDSVDSPR |
| 601 | TPTTGIRGGV TFEVSGLGGT YHFIKLSLNS SIYRKLTRKG |
| | ILKIKGEAQF |
| 651 | IKPYSNTTAE GVPVSERFFL GGETTVRGYK SFIIGPKYSA |
| | TEPQGGLSSL |
| 701 | LISEEFQYPL IRQPNISAFV FLDSGFVGLQ EYKISLKDLR |
| | SSAGFGLRFD |
| 751 | VMNNVRVMLG FGWPFRPTET LNGEKIDVSQ RFFFALGGMF * |

A predicted signal peptide is highlighted.

The cp6576 nucleotide sequence <SEQ ID 148> is:

| | |
|---|---|
| 1 | ATGCTCATCA TGCGAAATAA AGTTATCTTG CAAATATCTA |
| | TTCTAGCGTT |
| 51 | AATCCAAACC CCTTTAACTT TATTTTCTAC TGAAAAAGTT |
| | AAAGAAGGCC |
| 101 | ATGTGGTGGT AGACTCTATC ACAATCATAA CGGAAGGAGA |
| | AAATGCTTCA |
| 151 | AATAAACATC CCTTACCCAA ATTAAAGACC AGAAGTGGGG |
| | CTCTTTTTTC |
| 201 | TCAATTAGAT TTTGATGAAG ACTTGAGAAT TCTAGCTAAA |
| | GAATACGACT |
| 251 | CTGTTGAGCC TAAAGTAGAA TTTTCTGAAG GGAAAACTAA |
| | CATAGCCCTT |
| 301 | CACCTAATAG CTAAACCCTC AATTCGAAAT ATTCATATCT |
| | CAGGAAATCA |
| 351 | AGTCGTTCCT GAACATAAAA TTCTTAAAAC CCTACAAATT |
| | TACCGTAATG |
| 401 | ATCTCTTTGA ACGAGAAAAA TTTCTTAAGG GTCTTGATGA |
| | TCTAAGAACG |
| 451 | TATTATCTCA AGCGAGGATA TTTCGCATCC AGTGTAGACT |
| | ACAGTCTGGA |
| 501 | ACACAATCAA GAAAAAGGTC ACATCGATGT TTTAATTAAA |
| | ATCAATGAAG |
| 551 | GTCCTTGCGG GAAAATTAAA CAGCTTACGT TCTCAGGAAT |
| | CTCTCGATCA |
| 601 | GAAAAATCAG ATATCCAAGA ATTTATTCAA ACCAAGCAGC |
| | ACTCTACAAC |
| 651 | TACAAGTTGG TTTACTGGAG CTGGACTCTA TCACCCAGAT |
| | ATTGTTGAAC |
| 701 | AAGATAGCTT GGCAATTACG AATTACCTAC ATAATAACGG |
| | GTACGCTGAT |
| 751 | GCTATAGTCA ACTCTCACTA TGACCTTGAC GACAAAGGGA |
| | ATATTCTTCT |
| 801 | TTACATGGAT ATTGATCGAG GGTCGCGATA TACCTTAGGA |
| | CACGTCCATA |
| 851 | TCCAAGGGTT TGAGGTTTTG CCAAAACGCC TTATAGAAAA |
| | GCAATCCCAA |
| 901 | GTCGGCCCCA ATGATCTTTA TTGCCCCGAT AAAATATGGG |
| | ATGGGGCTCA |
| 951 | TAAGATCAAA CAAACTTATG CAAAGTATGG CTACATCAAT |
| | ACCAATGTAG |
| 1001 | ACGTTCTCTT CATCCCTCAC GCAACCCGCC CTATTTATGA |
| | TGTAACTTAT |
| 1051 | GAGGTAAGTG AAGGGTCTCC TTATAAAGTT GGGTTAATTA |
| | AAATTACTGG |
| 1101 | GAATACCCAT ACAAAATCTG ACGTTATTTT ACACGAAACC |
| | AGTCTCTTCC |
| 1151 | CAGGAGATAC ATTCAATCGC TTAAAGCTAG AAGATACTGA |
| | GCAACGTTTA |
| 1201 | AGAAATACAG GCTACTTCCA AAGCGTTAGT GTCTATACAG |
| | TTCGTTCTCA |
| 1251 | ACTTGATCCT ATGGGCAATG CGGATCAATA CCGAGATATT |
| | TTTGTAGAAG |
| 1301 | TCAAAGAAAC AACAACAGGA AACTTAGGCT TATTCTTAGG |
| | ATTTAGTTCT |
| 1351 | CTTGACAATC TTTTTGGAGG AATTGAACTA TCTGAAAGTA |
| | ATTTTGATCT |
| 1401 | ATTTGGAGCT AGAAATATAT TTTCTAAAGG TTTTCGTTGT |
| | CTAAGAGGCG |
| 1451 | GTGGAGAACA TCTATTCTTA AAAGCCAACT TCGGGGACAA |
| | AGTCACAGAC |
| 1501 | TATACTTTGA AGTGGACCAA ACCTCATTTT CTAAACACTC |
| | CTTGGATTTT |
| 1551 | AGGAATTGAA TTAGATAAAT CAATTAACAG AGCATTATCT |
| | AAAGATTATG |
| 1601 | CTGTCCAAAC CTATGGCGGG AACGTCAGCA CAACGTATAT |
| | CTTGAACGAA |
| 1651 | CACCTGAAAT ACGGTCTATT TTATCGAGGA AGTCAAACGA |
| | GTTTACATGA |
| 1701 | AAAACGTAAG TTCCTCCTAG GGCCAAATAT AGACAGCAAT |
| | AAAGGATTTG |
| 1751 | TCTCTGCTGC AGGTGTCAAC TTGAATTACG ATTCTGTAGA |
| | TAGTCCTAGA |
| 1801 | ACTCCAACTA CAGGGATTCG CGGGGGGGTG ACTTTTGAGG |
| | TTTCTGGTTT |
| 1851 | GGGAGGAACT TATCATTTTA CAAAACTCTC TTTAAACAGC |
| | TCTATCTATA |
| 1901 | GAAAACTTAC GCGTAAAGGT ATTTTGAAAA TCAAAGGGGA |
| | AGCTCAATTT |
| 1951 | ATTAAACCCT ATAGCAATAC TACAGCTGAA GGAGTTCCTG |
| | TCAGTGAGCG |
| 2001 | CTTCTTCCTA GGTGGAGAGA CTACAGTTCG GGGATATAAA |
| | TCCTTTATTA |
| 2051 | TCGGTCCAAA ATACTCTGCT ACAGAACCTC AGGGAGGACT |
| | CTCTTCGCTC |
| 2101 | CTTATTTCAG AAGAGTTTCA ATACCCTCTC ATCAGACAAC |
| | CTAATATTAG |
| 2151 | TGCCTTTGTA TTCTTAGACT CAGGTTTTGT CGGTTTACAA |
| | GAGTATAAGA |
| 2201 | TTTCGTTAAA AGATCTACGT AGTAGTGCTG GATTTGGTCT |
| | GCGCTTCGAT |
| 2251 | GTAATGAATA ATGTTCCTGT TATGTTAGGA TTTGGTTGGC |
| | CCTTCCGTCC |
| 2301 | AACCGAGACT TTGAATGGAG AAAAAATTGA TGTATCTCAG |
| | CGATTCTTCT |
| 2351 | TTGCTTTAGG GGGCATGTTC TAA |

The PSORT algorithm predicts outer membrane (0.7658).

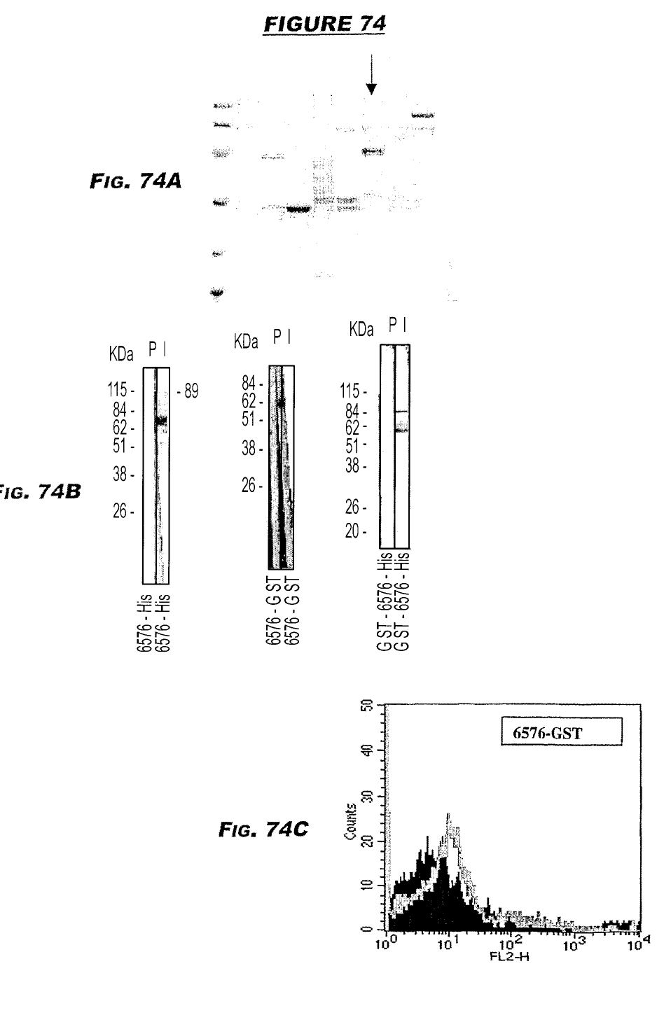

The protein was expressed in E. coli and purified as GST-fusion (FIG. 74A), his-tag and his-tag/GST-fusion products. The recombinant proteins were used to immunise mice, whose sera were used in a Western blot (FIG. 74B) and for FACS analysis (FIG. 74C).

The cp6576 protein was also identified in the 2D-PAGE experiment (Cpn0300).

These experiments show that cp6576 is a surface-exposed and immunoaccessible protein, and that it is a useful immunogen. These properties are not evident from the sequence alone.

### Example 75

The following C. pneumoniae protein (PID 4376607) was expressed <SEQ ID 149; cp6607>:

| | |
|---|---|
| 1 | MNKRQKDKLK ICVIISTLIL VGIFARAPRG DTFKTFLKSE |
| | EAIIYSNQCN |
| 51 | EDMRKILCDA IEHADEEIFL RIYNLSEPKI QQSLTRQAQA |
| | KNKVTIYYQK |
| 101 | FKIPQILKQA SNVTLVEQPP AGRKLMHQKA LSIDKKDAWL |
| | GSANYTLLSL |
| 151 | RLDNNLILGM HSSELCDLII TNTSGDFSIK DQTGKYFVLP |
| | QDRKIAIQAV |
| 201 | LEKIQTAQKT IQVAMFALTH SEIIQALHQA KQRGIHVDII |
| | IDRSHSKLTF |
| 251 | KQLRQLNINK DFVSINTAPC TLHHKFAVID NKTLLAGSIN |
| | WSKGRFSLND |
| 301 | ESLIILENLT KQQNQCLRMI WKDLAKHSEH PTVDDEEKEI |
| | IEKSLPVEFQ |
| 351 | EAA* |

A predicted signal peptide is highlighted.

The cp6607 nucleotide sequence <SEQ ID 150> is:

| | |
|---|---|
| 1 | ATGAATAAAA GACAAAAAGA TAAATTAAAA ATCTGTGTTA |
| | TTATTAGCAC |
| 51 | GTTGATTTTA GTAGGAATTT TTGCAAGAGC TCCTCGTGGT |
| | GACACTTTTA |
| 101 | AGACTTTTTT AAAGTCTGAA GAAGCTATCA TCTACTCAAA |
| | TCAATGCAAT |
| 151 | GAGGACATGC GTAAAATTCT ATGCGATGCT ATAGAACACG |
| | CTGATGAAGA |
| 201 | GATCTTCCTA CGTATTTATA ACCTCTCAGA ACCCAAGATC |
| | CAACAGAGTT |
| 251 | TAACTCGACA AGCTCAAGCA AAAAACAAAG TTACGATCTA |
| | CTATCAAAAA |
| 301 | TTTAAAATTC CCCAAATCTT AAAGCAAGCC AGCAATGTAA |
| | CTTTAGTCGA |
| 351 | GCAACCTCCA GCAGGGCGTA AACTGATGCA TCAAAAAGCT |
| | CTTTCCATAG |
| 401 | ATAAGAAAGA TCGTTGGCTA GGATCTGCGA ACTACACCAA |
| | TCTTTCTCTA |
| 451 | CGTTTAGATA ATAATCTCAT TCTAGGAATG CATAGCTCGG |
| | AGCTCTGTGA |
| 501 | TCTCATTATC ACAAATACCT CTGGAGACTT TTCTATAAAG |
| | GATCAAACAG |
| 551 | GAAAGTATTT TCTTCTTCCT CAAGATCGTA AAATTGCAAT |
| | ACAAGCTGTA |
| 601 | CTCGAAAAAA TCCAGACAGC TCAGAAAACC ATCCAAGTTG |
| | CTATGTTTGC |
| 651 | TCTGACCCAC TCGGAGATTA TTCAAGCCTT ACATCAAGCA |
| | AAACAACGAG |
| 701 | GAATCCATGT AGATATTATC ATTGATAGAA GTCATAGCAA |
| | ACTTACTTTT |
| 751 | AAGCAATTAC GACAATTAAA TATCAATAAA GACTTTGTTT |
| | CTATAAATAC |
| 801 | CGCACCCTGT ACTCTTCACC ATAAGTTTGC AGTTATAGAT |
| | AATAAAACTC |
| 851 | TACTTGCAGG ATCTATAAAT TGGTCTAAAG GAAGATTCTC |
| | CTTAAATGAT |
| 901 | GAAAGCTTGA TCATACTGGA AAACCTGACC AAACAACAAA |
| | ATCAGAAACT |
| 951 | TCGAATGATT TGGAAAGATC TAGCTAAGCA TTCAGAACAT |
| | CCTACAGTAG |
| 1001 | ACGATGAAGA AAAAGAAATT ATAGAAAAAA GTCTTCCAGT |
| | AGAAGAGCAA |
| 1051 | GAAGCAGCGT GA |

The PSORT algorithm predicts periplasmic (0.934).

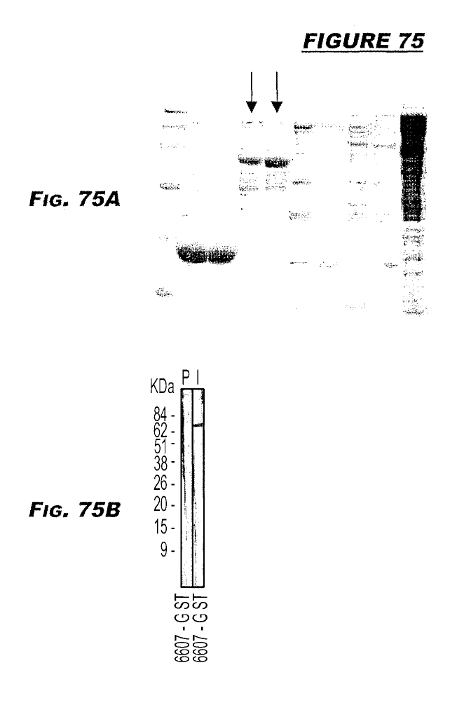

The protein was expressed in E. coli and purified as a his-tagged product (FIG. 75A) and also as a GST-fusion. The GST-fusion protein was used to immunise mice, whose sera were used in a Western blot (FIG. 75B) and for FACS analysis.

These experiments show that cp6607 is a surface-exposed and immunoaccessible protein, and that it is a useful immunogen. These properties are not evident from the sequence alone.

### Example 76

The following C. pneumoniae protein (PID 4376624) was expressed <SEQ ID 151; cp6624>:

| | |
|---|---|
| 1 | MDAKMGYIFK VMRWIFCFVA CGITFGCTNS GFQNANSRPC |
| | ILSMNRMIHD |
| 51 | CVERVVGNRL ATAVLIKGSL DPHAYEMVKG DKDKIAGSAV |
| | IFCNGLGLEH |
| 101 | TLSLRKHLEN NPNSVKLGER LIARGAFVPL EEDGICDPHI |
| | WMDLSIWKEA |
| 151 | VIEITEVLIE KFPEWSAEFK ANSEELVCEM SILDSWAKQC |
| | LSTIPENLRY |
| 201 | LVSGHNAFSY FTRRYLATPE EVASGAWRSR CISPEGLSPE |
| | AQISVRDIMA |
| 251 | VVDYINEHDV SVVFPEDTLN QDALKKIVSS LKKSHLVRLA |
| | QKPLYSDNVD |
| 301 | DNYFSTFKHN VCLITEELGG VALECQR* |

The cp6624 nucleotide sequence <SEQ ID 152> is:

| | |
|---|---|
| 1 | ATGGATGCGA AAATGGGATA TATATTTAAA GTGATGCGTT |
| | GGATTTTCTG |
| 51 | TTTCGTGGCA TGTGGTATAA CTTTTGGATG TACCAATTCT |
| | GGGTTTCAGA |
| 101 | ATGCAAATTC AGCTCCTTGT ATACTATCCA TGAATCGCAT |
| | GATTCATGAT |
| 151 | TGTGTTGAAA GAGTCGTGGG GAATAGGCTT GCTACCGCTG |
| | TTTTGATCAA |
| 201 | TGGATCCTTA GACCCTCATG CGTATGAGAT GGTTAAAGGG |
| | GATAAGGACA |
| 251 | AGATTGCTGG AAGTGCCGTA ATTTTTTGTA ACGGCCTGGG |
| | TCTTGAGCAT |
| 301 | ACATTAAGTT TGCGGAAGCA TTTAGAAAAT AATCCCAATA |
| | GTGTCAAGTT |
| 351 | AGGGGAGCGG TTGATAGCGC GTGGGGCCTT TGTTCCTCTA |
| | GAAGAAGACG |
| 401 | GTATTTGCGA TCCTCATATC TGGATGGATC TTTCTATTTG |
| | GAAGGAAGCT |
| 451 | GTCATAGAAA TTACAGAAGT TCTCATTGAA AAGTTCCCTG |
| | AATGGTCTGC |
| 501 | TGAATTTAAA GCAAATAGTG AGGAACTTGT TTGTGAAATG |
| | TCTATTTTAG |
| 551 | ATTCTTGGGC GAAACAATGC TTGAGCACAA TTCCTGAAAA |
| | TTTACGGTAT |
| 601 | CTTGTCTCAG GTCATAATGC GTTCAGTTAC TTTACACGTC |
| | GCTATTTAGC |
| 651 | TACTCCTGAA GAAGTGGCTT CCGGAGCATG GAGGTCTCGT |
| | TGTATTTCTC |
| 701 | CTGAGGGTCT ATCTCCAGAA GCTCAAATCA GTGTTCGTGA |
| | TATTATGGCG |
| 751 | GTTGTAGATT ATATTAATGA GCATGATGTC AGTGTGGTTT |
| | TCCCTGAGGA |
| 801 | TACTCTGAAC CAAGATGCGT TGAAAAAAAT TGTTTCTTCT |
| | CTGAAGAAAA |
| 851 | GTCATTTAGT TCGTCTAGCT CAAAAACCAT TGTATAGTGA |
| | TAATGTGGAC |
| 901 | GACAATTATT TTAGCACCTT TAAACATAAT GTCTGCCTTA |
| | TCACAGAAGA |
| 951 | ATTAGGAGGG GTGGCTCTTG AATGTCAAAG ATGA |

The PSORT algorithm predicts inner membrane (0.168).

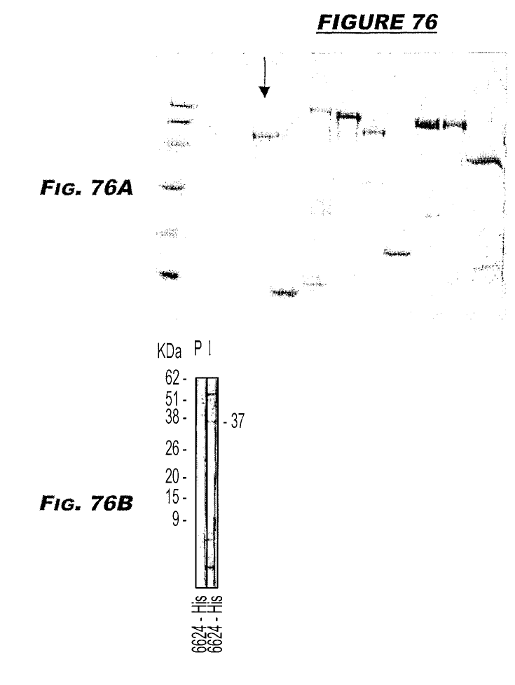

The protein was expressed in E. coli and purified as a his-tag product (FIG. 76A). The recombinant protein was used to immunise mice, whose sera were used in a Western blot (FIG. 76B) and for FACS analysis.

The cp6624 protein was also identified in the 2D-PAGE experiment.

These experiments show that cp6624 is a surface-exposed and immunoaccessible protein, and that it is a useful immunogen. These properties are not evident from the sequence alone.

### Example 77

The following C. pneumoniae protein (PID 4376728) was expressed <SEQ ID 153; cp6728>:

| | |
|---|---|
| 1 | MKSSVSWLFF SSIPLFSSLS IVAAEVTLDS SNNSYDGSNG |
| | TTFTVESTTD |
| 51 | AAAGTTYSLL SDVSFQNAGA LGIPLASGCF LEAGGDLTFQ |
| | GNQHALKFAF |
| 101 | INAGSSAGTV ASTSAADKNL LENDFSRLSI ISCPSLLLSP |
| | TGQCALKSVG |
| 151 | NLSLTGNSQI IFTQNFSSDN GGVINTKNFL LSGTSQFASF |
| | SRNQAFTGKQ |
| 201 | GGVVYATGTI TIENSPGIVS FSQNLAKGSG GALYSTDNCS |
| | ITDNFQVIFD |
| 251 | GNSAWEAAQA QGGAICCTTT DKTVTLTGNK NLSFTNNTAL |
| | TYGGAISGLK |
| 301 | VSISAGGPTL FQSNISGSSA GQGGGGAINI ASAGELALSA |
| | TSGDITFNNN |
| 351 | QVTNGSTSTR NAINIIDTAK VTSIRAATGQ SIYFYDPITN |
| | PGTAASTDTL |
| 401 | NLNLADANSE IEYGGAIVFS GEKLSPTEKA IAANVTSTIR |
| | QPAVLARGDL |
| 451 | VLRDGVTVTF KDLTQSPGSR ILMDGGTTLS AKEANLSLNG |
| | LAVNLSSLDG |
| 501 | TNKAALKTEA ADKNISLSGT IALIDTEGSF YENHNLKSAS |
| | TYPLLELTTA |
| 551 | GANGTITLGA LSTLTLQEPE THYGYQGNWQ LSWANATSSK |
| | IGSINWTRTG |
| 601 | YIPSPERKSN LPLNSLWGNF IDIRSINQLI ETKSSGEPFE |
| | RELWLSGIAN |
| 651 | FFYRDSMPTR HGFRHISGGY ALGITATTPA EDQLTFAFCQ |
| | LFARDRNHIT |
| 701 | GKNHGDTYGA SLYFHHTEGL FDIANFLWGK ATRAPWVLSE |
| | ISQIIPLSFD |
| 751 | AKFSYLHTDN HMKTYYTDNS IIKGSWRNDA FCADLGASLP |
| | FVISVPYLLK |
| 801 | EVEPFVKVQY IYAHQQDFYE RHAEGRAFNK SELINVEIPI |
| | GVTFERDSKS |
| 851 | EKGTYDLTLM YILDAYRRNP KCQTSLIASD ANWMAYGTNL |
| | ARQGFSVRAA |
| 901 | NHFQVNPHME LFGQFAFEVR SSSRNYNTNL GSKFCF* |

The cp6728 nucleotide sequence <SEQ ID 154> is:

| | |
|---|---|
| 1 | ATGAAGTCCT CTGTCTCTTG GTTGTTCTTT TCTTCAATCC |
| | CGCTCTTTTC |
| 51 | ATCGCTCTCT ATAGTCGCGG CAGAGGTGAC CTTAGATAGC |
| | AGCAATAATA |
| 101 | GCTATGATGG ATCTAACGGA ACTACCTTCA CGGTCTTTTC |
| | CACTACGGAC |
| 151 | GCTGCTGCAG GAACTACCTA TTCCTTACTT TCCGACGTAT |
| | CCTTTCAAAA |
| 201 | TGCAGGGGCT TTAGGAATTC CCTTAGCCTC AGGATGCTTC |
| | CTAGAAGCGG |
| 251 | GCGGCGATCT TACTTTCCAA GGAAATCAAC ATGCACTGAA |
| | GTTTGCATTT |
| 301 | ATCAATGCGG GCTCTAGCGC TGGAACTGTA GCCAGTACCT |
| | CAGCAGCAGA |
| 351 | TAAGAATCTT CTCTTTAATG ATTTTTCTAG ACTCTCTATT |
| | ATCTCTTGTC |
| 401 | CCTCTCTTCT TCTCTCTCCT ACTGGACAAT GTGCTTTAAA |
| | ATCTGTGGGG |
| 451 | AATCTATCTC TAACTGGCAA TTCCCAAATT ATATTTACTC |
| | AGAACTTCTC |
| 501 | GTCAGATAAC GGCGGTGTTA TCAATACGAA AAACTTCTTA |
| | TTATCAGGGA |
| 551 | CATCTCAGTT TGCGAGCTTT TCGAGAAACC AAGCCTTCAC |
| | AGGGAAGCAA |
| 601 | GGCGGTGTAG TTTACGCTAC AGGAACTATA ACTATCGAGA |
| | ACAGCCCTGG |
| 651 | GATAGTTTCC TTCTCTCAAA ACCTAGCGAA AGGATCTGGC |
| | GGTGCTCTGT |
| 701 | ACAGCACTGA CAACTGTTCG ATTACAGATA ACTTTCAAGT |
| | GATCTTTGAC |
| 751 | GGCAATAGTG CTTGGGAAGC CGCTCAAGCT CAGGGCGGGG |
| | CTATTTGTTG |
| 801 | CACTACGACA GATAAAACAG TGACTCTTAC TGGGAACAAA |
| | AACCTCTCTT |
| 851 | TCACAAATAA TACAGCATTG ACATATGGCG GAGCCATCTC |
| | TGGACTCAAG |
| 901 | GTCAGTATTT CCGCTGGAGG TCCTACTCTA TTTCAAAGTA |
| | ATATCTCAGG |
| 951 | AAGTAGCGCC GGTCAGGGAG GAGGAGGAGC GATCAATATA |
| | GCATCTGCTG |
| 1001 | GGGAACTCGC TCTCTCTGCT ACTTCTGGAG ATATTACCTT |
| | CAATAACAAC |
| 1051 | CAAGTCACCA ACGGAAGCAC AAGTACAAGA AACGCAATAA |
| | ATATCATTGA |
| 1101 | TACCGCTAAA GTCACATCGA TACGAGCTGC TACGGGGCAA |
| | TCTATCTATT |
| 1151 | TCTATGATCC CATCACAAAT CCAGGAACCG CAGCTTCTAC |
| | CGACACATTG |
| 1201 | ACCTTAAACT TAGCAGATGC GAACAGTGAG ATGGAGTATG |
| | GGGGTGCGAT |
| 1251 | TGTCTTTTCT GGAGAAAAGC TTTCCCCTAC AGAAAAAGCA |
| | ATCGCTGCAA |
| 1301 | ACGTCACCTC TACTATCCGA CACCCTGCAG TATTAGCGCG |
| | GGGAGATCTT |
| 1351 | GTACTTCGTG ATGGAGTCAC CGTACCTTTC AAGGATCTGA |
| | CTCAAAGTCC |
| 1401 | AGGATCCCGC ATCTTACTGG ATGGGGGGAC TACACTTAGT |
| | GCTAAAGAGG |
| 1451 | CACATCTTTC GCTTACTGGC TTAGCAGTAA ATCTCTCCTC |
| | TTTAGATGGA |
| 1501 | ACCAACAAGG CAGCTTTAAA AACAGAAGCT GCAGATAAAA |
| | ATATCAGCCT |
| 1551 | ATCGGGAACG ATTGCGCTTA TTGACACGGA AGGGTCATTC |
| | TATGAGAATC |
| 1601 | ATAACTTAAA AAGTGCTAGT ACCTATCCTC TTCTTGAACT |
| | TACCACCGCA |
| 1651 | GGAGCCAACG GAACGATTAC TCTGGGAGCT CTTTCTACCC |
| | TGACTCTTCA |
| 1701 | AGAACCTGAA ACCCACTACG GGTATCAAGG AAACTGGCAG |
| | TTGTCTTGGG |
| 1751 | CAAATGCAAC ATCCTCAAAA ATAGGAAGCA TCAACTGGAC |
| | CCGTACAGGA |
| 1801 | TACATTCCTA GTCCTGAGAG AAAAAGTAAT CTCCCTCTAA |
| | ATAGCTTATG |
| 1851 | GGGAAACTTT ATAGATATAC GCTCGATCAA TCAGCTTATA |
| | GAACCCAAGT |
| 1901 | CCAGTGGGGA GCCTTTTGAG CGTGAGCTAT GGCTTTCAGG |
| | AATTGCGAAT |
| 1951 | TTCTTCTATA GAGATTCTAT GCCCACCCGC CATGGTTTCC |
| | GCCATATCAG |
| 2001 | CGGGGGTTAT GCACTAGGGA TCACAGCAAC AACTCCTGCC |
| | GAGGATCAGC |
| 2051 | TTACTTTTGC CTTCTGCCAG CTCTTTGCTA GAGATCGCAA |
| | TCATATTACA |
| 2101 | GGTAAGAACC ACGGAGATAC TTACGGTGCC TCTTTGTATT |
| | TCCACCATAC |
| 2151 | AGAAGGGCTC TTCGACATCG CCAATTTCCT CTGGGGAAAA |
| | GCAACCCGAG |
| 2201 | CTCCCTGGGT GCTCTCTGAG ATCTCCCAGA TCATTCCTTT |
| | ATCGTTCGAT |
| 2251 | GCTAAATTCA GTTATCTCCA TACAGACAAC CACATGAAGA |
| | CATATTATAC |
| 2301 | CGATAACTCT ATCATCAAGG GTTCTTGGAG AAACGATGCC |
| | TTCTGTGCAG |
| 2351 | ATCTTGGAGC TAGCCTGCCT TTTGTTATTT CCGTTCCGTA |
| | TCTTCTGAAA |
| 2401 | GAAGTCGAAC CTTTTGTCAA AGTACAGTAT ATCTATGCGC |
| | ATCAGCAAGA |
| 2451 | CTTCTACGAG CTGCATGCTG AAGGACGCGC TTTCAATAAA |
| | AGCGAGCTTA |
| 2501 | TCAACGTAGA GATTCCTATA GGCGTCACCT TCGAAAGAGA |
| | CTCAAAATCA |
| 2551 | GAAAAGGGAA CTTACGATCT TACTCTTATG TATATACTCG |
| | ATGCTTACCG |
| 2601 | ACGCAATCCT AAATGTCAAA CTTCCCTAAT AGCTAGCGAT |
| | GCTAACTGGA |
| 2651 | TGGCCTATGG TACCAACCTC GCACGACAAG GTTTTTCTGT |
| | TCGTGCTGCG |
| 2701 | AACCATTTCC AAGTGAACCC CCACATGGAA ATCTTCGGTC |
| | AATTCGCTTT |
| 2751 | TGAAGTACGA AGTTCTTCAC GAAATTATAA TACAAACCTA |
| | GGCTCTAAGT |
| 2801 | TTTGTTTCTA G |

The PSORT algorithm predicts inner membrane (0.187).

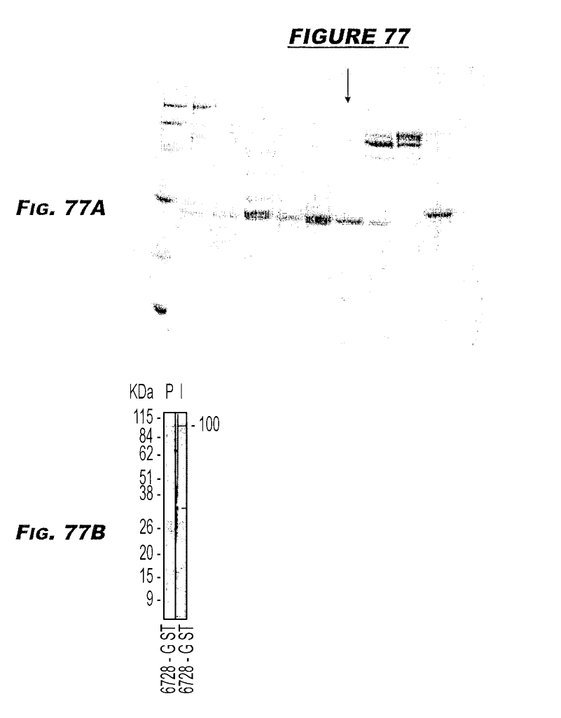

The protein was expressed in E. coli and purified as a GST-fusion product (FIG. 77A). The recombinant protein was used to immunise mice, whose sera were used in a Western blot (FIG. 77B) and for FACS analysis.

The cp6728 protein was also identified in the 2D-PAGE experiment.

These experiments show that cp6728 is a surface-exposed and immunoaccessible protein, and that it is a useful immunogen. These properties are not evident from the sequence alone.

### Example 78

The following C. pneumoniae protein (PID 4376847) was expressed <SEQ ID 155; cp6847>:

| | |
|---|---|
| 1 | MFVMKKLVRL CVVLISLLPN VLFSSDLLRE EGIKKMMDKL |
| | IEYHVDAQEV |
| 51 | STDILSRSLS SYIQSFDPHK SYLSNQEVAV FLQSPETKKR |
| | LLKVYKAGNF |
| 101 | AIYRNINQLI HESIIRARQW RNEWVKNPKE LVLEASSYQI |
| | SKQPMQWSKS |
| 151 | LDEVKQRQRA LLLSYLSLHL AGASSSRYEG KEEQLAALCL |
| | RQIENHENVY |
| 201 | LGINDHGVAM DRDEEAYQFH IRVVKALAHS LDAHTAYFSK |
| | DEALAMRIQL |
| 251 | EKGMCGIGVV LKEDIDGVVV REIIPGGPAA KSGDLQLGDI |
| | IYRVDGKDIE |
| 301 | HLSFRGVLDC LRGGHGSTVV LDIHRGESDH TIALRREKIL |
| | LEDRRVDVSY |
| 351 | EPYGDGVIGK VTLHSFYEGE NQVSSEQDLR RAIQGLKEKN |
| | LLGLVLDIRE |
| 401 | NTGGFLSQAI KVSGLFMTNG VVVVSRYADG TMKCYRTVSP |
| | KKFYDGPLAI |
| 451 | LVSKSSASAA EIVAQTLQDY GVALVVGDEQ TYGKGTIQHQ |
| | TITGDASQDD |
| 501 | CFKVTVGKYY SPSGKSTQLQ GVKSDILIPS LYAEDRLGER |
| | FLEHPLPADC |
| 551 | CDNVLHDPLT DLDTQTRPWF QKYYLPNLQK QETLWREMLP |
| | QLTKNSEQRL |
| 601 | SENSNFQAFL SQIKSSEKTD LSYGSNDLQL EESINILKDM |
| | ILLQQCRK* |

A predicted signal peptide is highlighted.

The cp6847 nucleotide sequence <SEQ ID 156> is:

| | |
|---|---|
| 1 | ATGTTCGTAA TGAAAAAACT TGTCCGTCTA TGCGTAGTTC |
| | TTCTTTCTTT |
| 51 | ACTTCCGAAT GTATTATTTT CTTCGGATCT TTTACGAGAA |
| | GAGGGCATCA |
| 101 | AAAAGATGAT GGACAAGCTG ATCGAGTATC ATGTCGATGC |
| | TCAAGAGGTT |
| 151 | TCTACGGATA TACTCTCGCG TTCTTTATCT AGTTACATTC |
| | AATCTTTTGA |
| 201 | TCCTCATAAA TCTTATCTTT CAAACCAAGA GGTTGCAGTT |
| | TTTCTACAGT |
| 251 | CTCCGGAAAC AAAGAAACGT CTCTTAAAGA ATTATAAGGC |
| | AGGCAACTTT |
| 301 | GCTATTTATC GCAACATCAA TCAATTAATT CATGAGAGTA |
| | TTCTTCGTGC |
| 351 | CAGGCAGTGG AGAAACGAAT GGGTTAAGAA TCCAAAAGAG |
| | CTTGTATTGG |
| 401 | AGGCATCCTC ATATCAGATA TCGAAGCAAC CTATGCAATG |
| | GAGCAAATCT |
| 451 | TTAGACGAAG TGAAGCAGAG ACAACGCGCT CTACTCCTTT |
| | CCTATCTTTC |
| 501 | TTTACATCTT GCTGGAGCTT CTTCCTCTCG TTATGAGGGT |
| | AAAGAAGAGC |
| 551 | AGCTTGCTGC TCTGTGTCTA CGTCAAATCG AGAACCATGA |
| | GAATGTATAT |
| 601 | TTAGGTATCA ACGATCATGG TGTTGCTATG GATCGGGATG |
| | AAGAAGCCTA |
| 651 | CCAATTCCAT ATCCGTGTTG TTAAAGCTTT AGCTCATAGC |
| | TTAGATGCAC |
| 701 | ATACGGCGTA TTTCAGTAAG GACGAAGCGT TGGCGATGCG |
| | AATCCAACTA |
| 751 | GAAAAAGGCA TCTGTGGAAT TGGTGTTGTT CTGAAGGAAG |
| | ATATTGATGG |
| 801 | AGTTGTTGTT AGAGAAATCA TTCCTGGGGG ACCTGCGGCT |
| | AAATCTGGGG |
| 851 | ATCTTCAGCT TGGAGATATC ATCTATCGGG TGGATGGCAA |
| | GGATATCGAG |
| 901 | CATCTTTCTT TCCGCGGTGT TTTAGATTGT TTACGTGGAG |
| | GTCATGGCTC |
| 951 | TACTGTAGTC TTAGATATCC ATCGTGGGGA GAGCGATCAT |
| | ACGATCGCCT |
| 1001 | TGAGAAGGGA GAAAATCCTT TTAGAAGACC GTCGTGTGGA |
| | TGTTTCCTAT |
| 1051 | GAGCCTTATG GAGATGGTGT GATTGGGAAA GTTACGTTAC |
| | ATTCTTTTTA |
| 1101 | TGAAGGAGAA AATCAGGTTT CTAGTGAACA AGATCTACGT |
| | CGAGCGATTC |
| 1151 | AGGGATTAAA GGAGAAGAAC CTTCTTGGAT TAGTTTTAGA |
| | TATCCGAGAA |
| 1201 | AATACGGGTG GATTTTTATC TCAAGCGATC AAAGTTTCTG |
| | GTTTATTTAT |
| 1251 | GACCAATGGC GTTGTGGTTG TATCTCGCTA TGCTGATGGT |
| | ACCATGAAGT |
| 1301 | GCTACCGCAC AGTATCTCCT AAAAAATTCT ATGATGGTCC |
| | TTTGGCTATT |
| 1351 | TTAGTATCTA AAAGTTCCGC ATCAGCAGCG GAGATTGTAG |
| | CACAAACTCT |
| 1401 | CCAAGATTAT GGAGTTGCTT TAGTTGTTGG AGATGAGCAG |
| | ACCTATGGGA |
| 1451 | AGGGAACGAT TCAGCATCAA ACAATTACTG GAGATGCCTC |
| | TCAGGACGAT |
| 1501 | TGTTTTAAGG TTACTGTAGG GAAATATTAT TCCCCTTCTG |
| | GGAAATCGAC |
| 1551 | TCAACTTCAG GGAGTAAAAT CCGATATTTT AATTCCTTCT |
| | CTCTATGCTG |
| 1601 | AAGATCGTCT AGGAGAGCGT TTTCTAGAGC ATCCCTTACC |
| | TGCAGATTGC |
| 1651 | TGTGATAATG TACTTCACGA TCCTCTCACG GAGTTGGATA |
| | CTCAAACACG |
| 1701 | TCCTTGGTTT CAAAAATACT ATCTTCCTAA TCTACAAAAG |
| | CAAGAGACTC |
| 1751 | TTTGGAGAGA GATGCTACCT CAGCTTACGA AAAACAGTGA |
| | GCAAAGGCTT |
| 1801 | TCTGAGAATT CGAATTTTCA GGCATTTTTG TCGCAGATAA |
| | AATCATCTGA |
| 1851 | AAAAACGGAC CTATCCTATG GTTCCAATGA TTTACAATTG |
| | GAAGAGTCGA |
| 1901 | TAAACATTTT GAAGGACATG ATTTTATTAC AACAGTGTAG |
| | AAAATAA |

The PSORT algorithm predicts periplasmic (0.932).

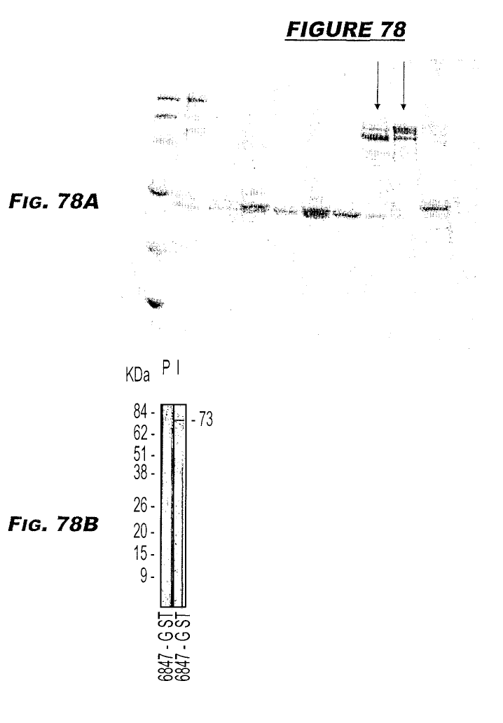

The protein was expressed in E. coli and purified as a GST-fusion product (FIG. 78A). The recombinant protein was used to immunise mice, whose sera were used in a Western blot (FIG. 78B) and for FACS analysis.

These experiments show that cp6847 is a surface-exposed and immunoaccessible protein, and that it is a useful immunogen. These properties are not evident from the sequence alone.

### Example 79

The following C. pneumoniae protein (PID 4376969) was expressed <SEQ ID 157; cp6969>:

| | |
|---|---|
| 1 | MRLFSLGTIY LFFSLALSSC CGYSILNSPY HLSSLGKSLL |
| | QERIFIAPIK |
| 51 | EDPHGQLCSA LTYELSKRSF AISGRSSCAG YTLKVELLNG |
| | IDKNIGFTYA |
| 101 | PNKLGDKTHR HFIVSNEGRL SLSAKVQLIN NDTQEVLIDQ |
| | CVARESVDFD |
| 151 | FEPDLTGANA HEFALGQFEM HSEAIKSARR ILSIRLAETI |
| | AQQVYYDLF* |

A predicted signal peptide is highlighted.

The cp6969 nucleotide sequence <SEQ ID 158> is:

| | |
|---|---|
| 1 | ATGAGATTGT TTTCTTTAGG CACGATTTAT CTTTTTTTTT |
| | CTCTAGCACT |
| 51 | TTCGTCATGC TGTGGTTACT CTATTTTAAA CAGCCCGTAT |
| | CACTTATCGT |
| 101 | CTTTAGGTAA GTCTTTATTA CAGGAAAGAA TTTTCATTGC |
| | TCCCATAAAA |
| 151 | GAAGATCCTC ATGGTCAGCT CTGCTCAGCT CTAACTTATG |
| | AGCTTAGTAA |
| 201 | GCGTTCTTTT GCTATCTCTG GAAGGAGTTC TTGCGCAGGC |
| | TATACTCTTA |
| 251 | AAGTAGAGCT TCTGAATGGT ATTGACAAGA ATATAGGTTT |
| | TACGTATGCC |
| 301 | CCAAATAAAC TCGGAGATAA GACTCACAGG CATTTTATAG |
| | TCTCTAATGA |
| 351 | AGGCAGACTA TCACTATCTG CAAAAGTACA GCTTATCAAT |
| | AATGACACTC |
| 401 | AAGAAGTCCT TATAGACCAA TGTGTTGCTC GAGAGTCTGT |
| | AGACTTTGAC |
| 451 | TTTGAGCCTG ACTTAGGAAC AGCAAACGCT CATGAATTTG |
| | CTTTAGGCCA |
| 501 | ATTTGAAATG CATAGTGAAG CCATAAAAAG TGCTCGCCGT |
| | ATACTATCTA |
| 551 | TACGCCTAGC CGAGACGATT GCTCAACAGG TATACTATGA |
| | CCTTTTTTGA |

The PSORT algorithm predicts inner membrane (0.126).

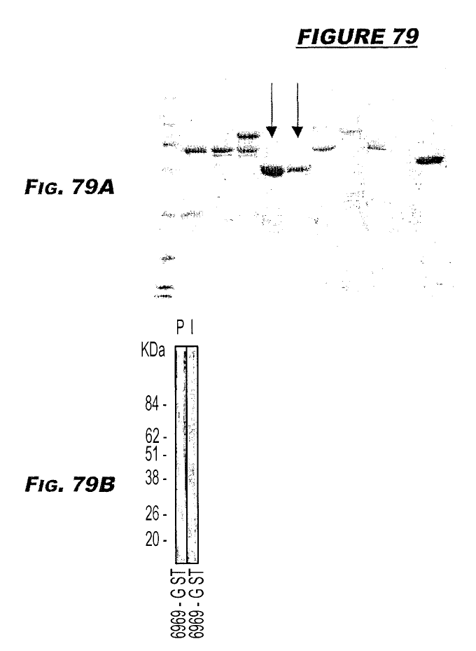

The protein was expressed in E. coli and purified as a GST-fusion product (FIG. 79A). The recombinant protein was used to immunise mice, whose sera were used in a Western blot (FIG. 79B) and for FACS analysis.

These experiments show that cp6969 is a surface-exposed and immunoaccessible protein, and that it is a useful immunogen. These properties are not evident from the sequence alone.

### Example 80

The following C. pneumoniae protein (PID 4377109) was expressed <SEQ ID 159; cp7109>:

| | |
|---|---|
| 1 | MKKTCCQNYR SIGVVFSVVL FVLTTQTLFA GHFIDIGTSG |
| | LYSWARGVSG |
| 51 | DGRVVVGYEG GNAFKYVDGE KFLLEGLVPR SEALVFKASY |
| | DGSVIIGISD |
| 101 | QDPSCRAVKW VNGALVDLGI FSEGMQSFAE GVSSDGKTIV |
| | GCLYSDDTET |
| 151 | NFAVKWDETG MVVLPNLPED RHSCAWDASE DGSVIVGDAM |
| | GSEEIAKAVY |
| 201 | WKDGEQHLLS NIPGAKRSSA HAVSKDGSFI VGEFISEENE |
| | VHAFVYHNGV |
| 251 | IKDIGTLGGD YSVATGVSRD GKVIVGHSTR TDGEYRAFKY |
| | VDGRMIDLGT |
| 301 | LGGSASFAFG VSDDGKTIVG KFETELGECH AFIYLDD* |

A predicted signal peptide is highlighted.

The cp7109 nucleotide sequence <SEQ ID 160> is:

| | |
|---|---|
| 1 | ATGAAAAAGA CATGTTGCCA AAATTACAGA TCGATAGGCG |
| | TTGTGTTCTC |
| 51 | TGTGGTACTT TTCGTTCTTA CAACACAGAC GCTGTTTGCA |
| | GGACATTTTA |
| 101 | TTGATATTGG AACTTCTGGA TTATATTCTT GGGCTCGAGG |
| | TGTATCTGGA |
| 151 | GATGGCCGCG TTGTCGTAGG TTATGAAGGT GGCAATGCAT |
| | TTAAATATGT |
| 201 | TGATGGTGAG AAATTTCTGT TAGAAGGTTT GGTCCCGAGA |
| | TCCGAGGCCT |
| 251 | TGGTATTTAA AGCTTCTTAT GATGGCTCTG TAATTATAGG |
| | AATCTCGGAT |
| 301 | CAAGATCCGT CTTGCCGCGC TGTGAAGTGG GTAAACGGTG |
| | CACTTGTTGA |
| 351 | TCTTGGAATA TTTTCTGAGG GAATCCAATC TTTTGCAGAG |
| | GGTGTTTCCA |
| 401 | GTGATGGAAA GACGATTGTA GGGTGCCTAT ATAGTGATGA |
| | TACAGAGACA |
| 451 | AACTTTGCTG TGAAGTGGGA TGAAACAGGA ATGGTTGTTC |
| | TCCCTAACTT |
| 501 | ACCAGAAGAT CGACATTCTT GCGCTTGGGA TGCCTCTGAA |
| | GATGGCTCTG |
| 551 | TGATTGTAGG GGACGCCATG GGTAGCGAGG AAATTGCCAA |
| | GGCAGTGTAC |
| 601 | TGGAAGGACG GTGAACAACA TCTGCTTTCT AATATCCCAG |
| | GAGCTAAAAG |
| 651 | ATCGTCAGCA CATGCAGTTT CTAAAGATGG ATCTTTTATC |
| | GTAGGCGAGT |
| 701 | TCATCAGTGA AGAAAATGAA GTTCATGCCT TTGTTTATCA |
| | CAACGGTGTT |
| 751 | ATCAAAGATA TCGGGACTTT AGGAGGAGAT TACTCTGTAG |
| | CAACTGGAGT |
| 801 | TTCTAGGGAT GGTAAGGTCA TCGTGGGTCA TTCTACAAGA |
| | ACAGATGGTG |
| 851 | AATACCGTGC ATTTAAATAT GTGGATGGAA GAATGATAGA |
| | TTTGGGGACT |
| 901 | TTAGGAGGTT CAGCATCTTT TGCTTTTGGT GTTTCTGACG |
| | ATGGCAAAAC |
| 951 | AATCGTAGGA AAATTTGAAA CAGAGCTAGG AGAATGTCAT |
| | GCCTTTATCT |
| 1001 | ACCTTGATGA TTAG |

The PSORT algorithm predicts outer membrane (0.887).

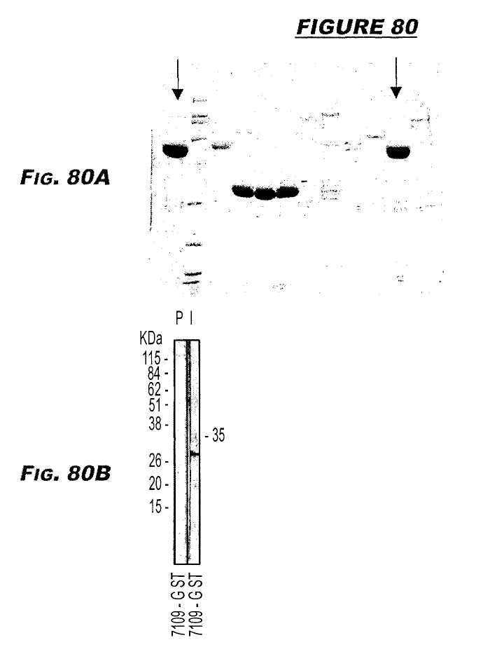

The protein was expressed in E. coli and purified as a GST-fusion product (FIG. 80A). The recombinant protein was used to immunise mice, whose sera were used in a Western blot (FIG. 80B) and for FACS analysis.

These experiments show that cp7109 is a surface-exposed and immunoaccessible protein, and that it is a useful immunogen. These properties are not evident from the sequence alone.

### Example 81

The following C. pneumoniae protein (PID 4377110) was expressed <SEQ ID 161; cp7110>:

| | |
|---|---|
| 1 | MAAIKQILRS MLSQSSLWMV LFSLYSLSGY CYVITDKPED |
| | DFHSSSAVKW |
| 51 | DHWGKTTLSR LSNKKASAKA VSGTGATTVG FIKDTWSRTY |
| | AVRWNYWGTK |
| 101 | ELPTSSWVKK SKATGISSDG SIIAGIVENE LSQSFAVTWK |
| | NNEMYLLPST |
| 151 | WAVQSKAYGI SSDGSVIVGS AKDAWSRTFA VKWTGHEAQV |
| | LPVGWAVKSV |
| 201 | ANSVSANGSI IVGSVQDASG ILYAVKWEGN TITHLGTLGG |
| | YSAIAKAVSN |
| 251 | NGKVIVGRSE TYYGEVHAFC HKNGVMSDLG TLGGSYSAAK |
| | GVSATGKVIV |
| 301 | GMSTTANGKL HAFKYVGGRM IDKGEYSWKE ACANAVSIDG |
| | EIIVGVQSE* |

A predicted signal peptide is highlighted.

The cp7110 nucleotide sequence <SEQ ID 162> is:

| | |
|---|---|
| 1 | ATGGCAGCTA TAAAACAAAT TTTACGTTCT ATGCTATCTC |
| | AGAGTAGCTT |
| 51 | ATGGATGGTC CTATTTTCAT TATATTCTCT ATCTGGTTAT |
| | TGCTATGTAA |
| 101 | TTACAGACAA ACCAGAAGAT GACTTCCATT CTTCATCCGC |
| | AGTAAAATGG |
| 151 | GATCATTGGG GAAAGACAAC TCTCTCAAGA TTATCAAATA |
| | AAAAAGCCTC |
| 201 | TGCAAAAGCT GTTTCAGGAA CTGGTGCTAC AACTGTCGGC |
| | TTTATAAAAG |
| 251 | ACACTTGGTC TCGAACATAC GCAGTAAGAT GGAATTATTG |
| | GGGGACCAAA |
| 301 | GAACTCCCTA CCAGCTCATG GGTAAAAAAA TCAAAAGCAA |
| | CAGGAATCTC |
| 351 | CTCTGATGGG TCTATAATCG CGGGGATTGT CGAGAATGAG |
| | CTTTCTCAAA |
| 401 | GTTTCGCAGT CACATGGAAA AACAATGAAA TGTATTTGCT |
| | CCCTTCCACA |
| 451 | TGGGCAGTGC AATCTAAAGC GTATGGAATT TCTTCTGATG |
| | GCTCTGTTAT |
| 501 | TGTAGGGAGT GCTAAGGATG CTTGGTCGCG AACTTTCGCT |
| | GTGAAGTGGA |
| 551 | CGGGACACGA GGCTCAGGTG TTACCAGTAG GCTGGGCTGT |
| | CAAATCTGTA |
| 601 | GCGAATTCTG TATCTGCCAA TGGATCTATA ATTGTAGGGT |
| | CTGTACAAGA |
| 651 | CGCCTCTGGA ATTCTTTATG CTGTAAAGTG GGAAGGGAAC |
| | ACTATTACAC |
| 701 | ATCTAGGAAC TTTAGGAGGC TATTCTGCCA TTGCAAAAGC |
| | TGTATCCAAT |
| 751 | AATGGCAAGG TCATTGTAGG GAGATCCGAA AGATATTATG |
| | GAGAGGTCCA |
| 801 | TGCTTTCTGT CATAAGAATG GCGTCATGTC AGACCTCGGC |
| | ACCCTCGGAG |
| 851 | GATCTTATTC TGCAGCTAAG GGAGTCTCTG CAACTGGAAA |
| | AGTTATTGTC |
| 901 | GGTATGTCCA CAACAGCAAA TGGGAAATTG CATGCCTTTA |
| | AATATGTCGG |
| 951 | TGGAAGAATG ATCGACTTAG GAGAGTATAG CTGGAAAGAA |
| | GCCTGTGCAA |
| 1001 | ACGCTGTTTC TATTCATGGA GAAATTATTG TTGGAGTCCA |
| | ATCAGAATAA |

The PSORT algorithm predicts outer membrane (0.827).

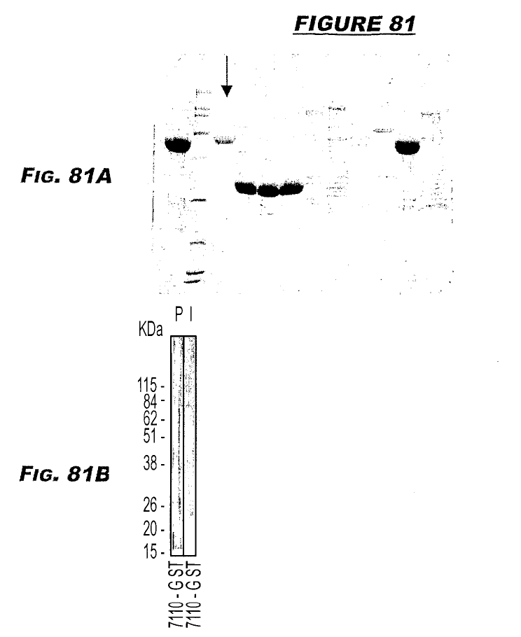

The protein was expressed in E. coli and purified as a GST-fusion product (FIG. 81A). The recombinant protein was used to immunise mice, whose sera were used in a Western blot (FIG. 81B) and for FACS analysis.

These experiments show that cp7110 is a surface-exposed and immunoaccessible protein, and that it is a useful immunogen. These properties are not evident from the sequence alone.

FIG. 191 shows a schematic representation of the structural relationships between of cp7105, cp7106, cp7107, cp7108, cp7109 and cp7110, each of which is identified herein. These six proteins may be grouped in a new family of related outer membrane-associated proteins. These proteins have a repeat structure in common (cf. the pmp family).

### Example 82

The following C. pneumoniae protein (PID 4377127) was expressed <SEQ ID 163; cp7127>:

| | |
|---|---|
| 1 | MVFFRNSLLH LVALSGMLCC SSGVALTIAE KMASLEHSGR |
| | GADDYEGMAS |
| 51 | FNANMREYSL QLSKLYEEAR KLRASGTEDE ALWKDLIRRI |
| | GEVRGYLREI |
| 101 | EELWAAEIRE KGGNLEDYAL WNHPETTIYN LVTDYGTEDS |
| | IYLIPQEIGA |
| 151 | IKIATLSKFV VPKESFEDCL TQILSRLGIG VRQVNSWIKE |
| | LYMMRKEGCS |
| 201 | VAGVFSSRKD LEALPETAYI GFVLNSNVDA HTNQHVLKKF |
| | INPETTHVDV |
| 251 | IAGRVWIFGS AGEVGELLKI YNFVQSESIR QEYRVIPLTK |
| | IDPGEMISIL |
| 301 | NAAFREDLTK DVSEESLGLR VVPLQYQGRS LFLSGTAALV |
| | QQALTLIREL |
| 351 | EEGIENPTDK TVFWYNVKHS DPQELAALLS QVHDVFSGEN |
| | KASVGAADGC |
| 401 | GSQLNASIQI DTTVSSSAKD GSVKYGNFIA DSKTGTLIMV |
| | VEKEVLPRIQ |
| 451 | MLLKKLDVPK KMVRIEVLLF ERKLAHEQKS GLNLLRLGEE |
| | VCKKGCSPSV |
| 501 | SWAGGTGILE FLFKGSTGSS IVPGYDLAYQ FLMAQEDVRI |
| | NASPSVVTMN |
| 551 | QTPARIAVVD EMSIAVSSDK DKAQYNRAQY GIMIKMLPVI |
| | NVGEEDGKSY |
| 601 | ITLETDITFD TTGKNHDDRP DVTRRNITNK VRIADGETVI |
| | IGGLRCKQMS |
| 651 | DSHDGIPFLG DIPGIGKLFG MSSTSDSLTE MFVFITPKIL |
| | ENPVEQQERK |
| 701 | EEALLSSRPG EREEYYQALA ASEAAARAAH KKLEMFPASG |
| | VSLSQVERQE |
| 751 | YDGC* |

A predicted signal peptide is highlighted.

The cp7127 nucleotide sequence <SEQ ID 164> is:

| | |
|---|---|
| 1 | ATGGTTTTTT TCCGTAATTC TTTACTGCAT TTAGTTGCCC |
| | TATCCGGAAT |
| 51 | GCTCTGTTGT TCTTCTGGAG TGGCTTTAAC CATAGCCGAG |
| | AAGATGGCTT |
| 101 | CTTTAGAGCA CTCGGGGAGA GGAGCAGACG ATTATGAGGG |
| | GATGGCTTCG |
| 151 | TTTAATGCCA ATATGAGGGA GTATAGCCTT CAGCTGAGCA |
| | AGTTGTATGA |
| 201 | GGAAGCACGA AAGCTACGCG CTTCTGGAAC TGAGGATGAA |
| | GCTCTGTGGA |
| 251 | AGGACTTAAT TCGACGGATT GGTGAGGTGC GAGGCTATCT |
| | TCGAGAGATC |
| 301 | GAGGAGCTTT GGGCTGCAGA AATTCGTGAG AAAGGGGGCA |
| | ATCTCGAGGA |
| 351 | CTACGCCCTC TGGAATCACC CAGAGACTAC GATTTACAAT |
| | CTTGTTACCG |
| 401 | ATTACGGAAC CGAAGACTCT ATTTATTTGA TTCCTCAAGA |
| | AATCGGAGCG |
| 451 | ATTAAAATCG CAACCTTATC GAAATTTGTA GTTCCTAAAG |
| | AGTCTTTCGA |
| 501 | AGACTGTCTC ACTCAGATCC TATCTCGCTT AGGTATTGGC |
| | GTGCGTCAGG |
| 551 | TCAATTCTTG GATTAAGGAA CTTTATATGA TGCGTAAGGA |
| | GGGCTGCAGT |
| 601 | GTTGCTGGAG TTTTTTCCTC CAGAAAAGAT TTAGAGGCGC |
| | TCCCAGAAAC |
| 651 | AGCCTATATT GGTTTTGTAT TGAATTCGAA CGTAGATGCG |
| | CATACCAATC |
| 701 | AACATGTCTT AAAAAAGTTC ATTAACCCTG AAACAACGCA |
| | TGTAGATGTG |
| 751 | ATTGCAGGAC GTGTGTGGAT TTTTGGTTCT GCGGGGGAAG |
| | TCGGCGAGCT |
| 801 | TCTGAAGATT TATAATTTTG TGCAGTCGGA GAGCATACGT |
| | CAAGAGTATC |
| 851 | GGGTGATTCC CTTAACTAAG ATCGATCCAG GGGAGATGAT |
| | TTCCATTCTC |
| 901 | AACGCAGCAT TTCGTGAGGA TCTGACTAAA GATGTTAGTG |
| | AAGAATCTTT |
| 951 | AGGCCTTCGT GTAGTTCCTT TACAGTATCA AGGGCGTTCG |
| | TTGTTTTTAA |
| 1001 | GTGGAACCGC GGCGTTAGTG CAGCAAGCGC TGACTCTCAT |
| | TCGAGAGCTT |
| 1051 | GAAGAAGGGA TTGAGAACCC TACGGATAAA ACAGTATTTT |
| | GGTATAACGT |
| 1101 | CAAGCACTCC GATCCCCAAG AGTTGGCGGC ATTGCTTTCC |
| | CAAGTCCATG |
| 1151 | ATGTCTTCTC TGGCGAGAAT AAGGCGAGTG TCGGAGCTGC |
| | AGATGGATGT |
| 1201 | GGGTCGCAAT TAAATGCCTC GATCCAAATT GATACTACAG |
| | TAAGTTCTTC |
| 1251 | TGCGAAAGAT GGCTCAGTGA AGTACGGAAA CTTCATCGCG |
| | GATTCTAAGA |
| 1301 | CAGGAACTCT GATTATGGTG GTTGAGAAAG AAGTTCTTCC |
| | ACGTATTCAG |
| 1351 | ATGCTACTTA AGAAACTAGA TGTCCCTAAA AAGATGGTCC |
| | GTATCGAGGT |
| 1401 | GCTGTTATTT GAAAGAAAAT TGGCACATGA GCAGAAATCT |
| | GGGTTAAATC |
| 1451 | TTCTACGTCT TGGTGAGGAA GTTTGTAAAA AAGGGTGCAG |
| | TCCTTCTGTG |
| 1501 | TCTTGGGCCG GGGGTACTGG CATACTAGAA TTTTTATTTA |
| | AAGGAAGTAC |
| 1551 | GGGATCTTCG ATAGTTCCTG GTTATGATCT CGCCTATCAA |
| | TTTTTAATGG |
| 1601 | CTCAAGAGGA CGTTCGGATT AATGCGAGTC CTTCTGTAGT |
| | TACTATGAAC |
| 1651 | CAAACCCCAG CACGGATTGC TGTTGTTGAT GAAATGTCAA |
| | TAGCGGTGTC |
| 1701 | TTCAGATAAA GATAAAGCGC AATACAATCG TGCGCAGTAC |
| | GGTATCATGA |
| 1751 | TAAAAATGCT CCCCGTAATT AATGTGGGAG AGGAAGACGG |
| | AAAAAGTTAC |
| 1801 | ATTACTTTAG AGACAGACAT CACCTTTGAT ACTACGGGAA |
| | AAAATCATGA |
| 1851 | TGATCGTCCT GATGTTACAA GGCGTAATAT TACTAATAAG |
| | GTGCGCATTG |
| 1901 | CTGACGGAGA GACTGTGATT ATTGGAGGTT TGCGTTGCAA |
| | ACAGATGTCA |
| 1951 | GATTCTCATG ATGGCATTCC TTTCCTTGGA GACATTCCTG |
| | GTATAGGGAA |
| 2001 | GTTATTTGGA ATGAGTTCCA CATCAGACAG TCTCACGGAG |
| | ATGTTTGTAT |
| 2051 | TTATCACTCC GAAGATCCTA GAAAATCCTG TAGAGCAACA |
| | AGAACGTAAA |
| 2101 | GAAGAAGCTT TACTCTCTTC GCGCCCTGGA GAGAGAGAAG |
| | AATACTATCA |
| 2151 | GGCTTTAGCA GGTAGTGAGG CTGCAGCACG AGCAGCTCAT |
| | AAAAAATTAG |
| 2201 | AGATGTTCCC GGCATCAGGA GTATCTTTAT CTCAGGTAGA |
| | GAGGCAAGAA |
| 2251 | TACGATGGCT GCTAG |

The PSORT algorithm predicts periplasmic (0.920).

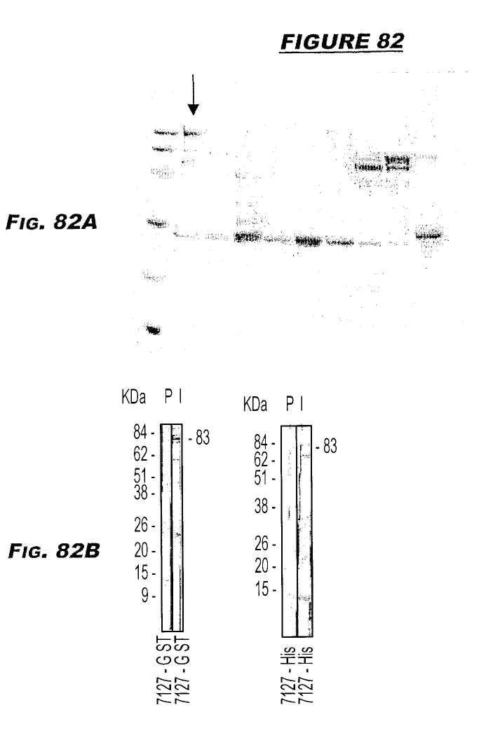

The protein was expressed in E. coli and purified as a GST-fusion product (FIG. 82A) and also in his-tagged form. The recombinant proteins were used to immunise mice, whose sera were used in a Western blot (FIG. 82B) and for FACS analysis.

These experiments show that cp7127 is a surface-exposed and immunoaccessible protein, and that it is a useful immunogen. These properties are not evident from the sequence alone.

### Example 83

The following C. pneumoniae protein (PID 4377133) was expressed <SEQ ID 165; cp7133>:

| | |
|---|---|
| 1 | MQPFIFTLLC LTSLVSLVAF DAANARKRCA CAQTIERGEN |
| | FFSIKRSACA |
| 51 | EIEYQEKSRH ASAIERISKD KGKVTPKQIA KVATKKKQRY |
| | RLLQVPFSRP |
| 101 | PNNSRYNLYA LLSEPPECYS DTASWYAIFI RLLRRAYVDT |
| | GNVPPGSEYA |
| 151 | IANALISNKQ EILERGAQLG PDVIETLTLP EEQAEIFYKM |
| | LKGSSNSQSL |
| 201 | LNFLHYEEKS LGHCKLNLIF MDPLLLEAVL DHPDAYRETS |
| | LLRDGIWEAV |
| 251 | KRQEHAIQEH GQAAALELFK TRTDFRLELR DKMQLLLSRY |
| | DLLPLLNKKM |
| 301 | FDYTLGSAGD YLFLVDPDTK AISRCRCPSK SIKL |

A predicted signal peptide is highlighted.

The cp7133 nucleotide sequence <SEQ ID 166> is:

| | |
|---|---|
| 1 | ATGCAACCTT TTATCTTTAC TTTACTGTGC TTGACATCTT |
| | TGGTTTCTTT |
| 51 | AGTCGCCTTT GATGCTGCGA ATGCTCGTAA ACGTTGTGCC |
| | TGTGCTCAAA |
| 101 | CTATAGAACG TGGAGAGAAC TTCTTTTCCA TAAAACGCTC |
| | TGCTTGTGCT |
| 151 | GAAATCGAAT ATCAAGAAAA ATCTCGCCAC GCCTCAGCAA |
| | TTGAAAGAAT |
| 201 | CTCAAAAGAT AAAGGCAAAG TCACTCCAAA GCAGATTGCG |
| | AAAGTAGCTA |
| 251 | CTAAGAAAAA GCAAAGATAC CGTTTATTGC AGGTTCCTTT |
| | TTCAAGGCCT |
| 301 | CCGAATAACT CAAGGTATAA CCTCTATGCT TTGCTTAGTG |
| | AACCTCCCGA |
| 351 | ATGCTATAGC GATACAGCAT CATGGTATGC TATTTTTATT |
| | CGGTTACTTC |
| 401 | GACGTGCTTA TGTAGACACG GGAAATGTAC CTCCTGGATC |
| | TGAGTATGCC |
| 451 | ATCGCTAATG CTTTGATAAG TAACAAACAA GAGATTTTAG |
| | AGAGGGGAGC |
| 501 | GCAGCTTGGA CCCGATGTTA TTGAAACTCT AACATTGCCT |
| | GAGGAACAAG |
| 551 | CCGAGATTTT TTATAAAATG CTCAAAGGGT CGTCAAACTC |
| | TCAGTCGCTA |
| 601 | CTGAATTTTC TGCATTATGA AGAGAAAAGC TTAGGCCACT |
| | GTAAGCTAAA |
| 651 | TCTGATCTTC ATGGATCCCC TACTGTTAGA AGCTGTTCTA |
| | GATCATCCCG |
| 701 | ATGCTTATAG GGAAACGTCG CTCCTGCGCG ATGGCATTTG |
| | GGAAGCGGTG |
| 751 | AAGCGTCAAG AACATGCCAT CCAAGAACAT GGCCAGGCAG |
| | CTGCTTTGGA |
| 801 | GCTTTTTAAA ACACGCACCG ACTTCCGCCT GGAGCTGCGA |
| | GATAAGATGC |
| 851 | AGTTACTTCT AAGTCGATAC GATTTGCTCC CCTTATTAAA |
| | TAAAAAAATG |
| 901 | TTCGACTACA CCTTAGGAAG TGCCGGAGAT TACTTATTTT |
| | TGGTAGACCC |
| 951 | AGATACTAAG GCAATTTCTC GATGTCGCTG CCCTTCAAAG |
| | AGTATTAAAT |
| 1001 | TATAA |

The PSORT algorithm predicts outer membrane (0.92).

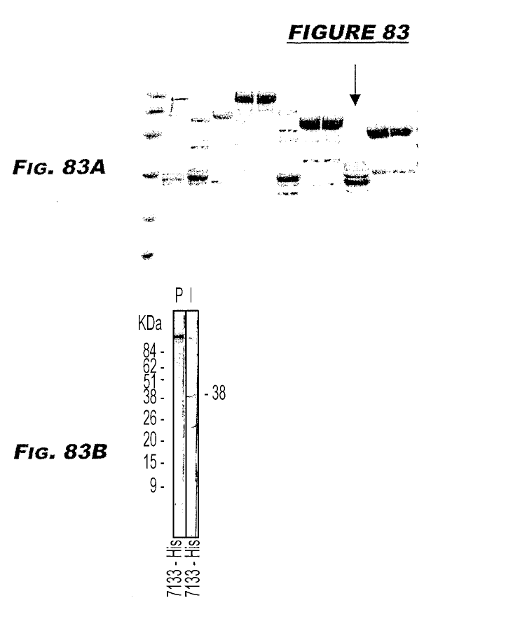

The protein was expressed in E. coli and purified as a GST-fusion product (FIG. 83A) and also in his-tagged form. The recombinant proteins were used to immunise mice, whose sera were used in a Western blot (FIG. 83B) and for FACS analysis.

These experiments show that cp7133 is a surface-exposed and immunoaccessible protein, and that it is a useful immunogen. These properties are not evident from the sequence alone.

### Example 84

The following C. pneumoniae protein (PID 4377222) was expressed <SEQ ID 167; cp7222>:

| | |
|---|---|
| 1 | MNRRDMVITA VVVNAILLVA LFVTSKRIGV KDYDEGFRNF |
| | ASSKVTQAVV |
| 51 | SEEKVIEKPV VAEVPSRPIA KETLAAQFIE SKPVIVTTPP |
| | VPVVSETPEV |
| 101 | PTVAVPPQPV RETVKEEQAP YATVVVKKGD FLERIARANH |
| | TTVAKLMQIN |
| 151 | DLTITQLKIG QVIKVPTSQD VSNEKTPQTQ TANPENYYIV |
| | QEGDSPWTIA |
| 201 | LRNHIRLDDL LKMNDLDEYK ARRLKPGDQL RIR* |

A predicted signal peptide is highlighted.

The cp7222 nucleotide sequence <SEQ ID 168> is:

| | |
|---|---|
| 1 | ATGAATCGTA GAGACATGGT AATAACAGCT GTCGTAGTGA |
| | ATGCTATATT |
| 51 | GCTTGTGGCT CTTTTCGTCA CATCAAAGCG TATTGGCGTC |
| | AAGGACTATG |
| 101 | ACGAGGGATT CCGTAATTTT GCTTCTAGCA AGGTTACACA |
| | AGCAGTAGTT |
| 151 | TCAGAAGAAA AAGTCATAGA AAAGCCTGTA GTCGCAGAAG |
| | TGCCTAGCCG |
| 201 | TCCTATCGCT AAAGAGACTC TAGCTGCACA GTTTATTGAA |
| | AGTAAGCCGG |
| 251 | TTATTGTAAC CACACCACCC GTGCCTGTTG TTAGCGAAAC |
| | CCCAGAAGTG |
| 301 | CCTACTGTGG CAGTTCCGCC TCAGCCTGTT CGTGAGACAG |
| | TAAAAGAGGA |
| 351 | ACAAGCTCCT TATGCTACTG TTGTAGTGAA AAAAGGAGAT |
| | TTTCTCGAAC |
| 401 | GCATTGCGAG AGCAAATCAT ACTACCGTTG CAAAATTGAT |
| | GCAGATCAAT |
| 451 | GATCTTACCA CCACCCAACT TAAAATTGGT CAGGTCATCA |
| | AAGTCCCTAC |
| 501 | GTCTCAAGAT GTCAGCAACG AAAAAACTCC TCAAACACAG |
| | ACCGCAAACC |
| 551 | CTGAAAATTA TTATATCGTC CAAGAAGGGG ATAGCCCGTG |
| | GACAATAGCA |
| 601 | TTGCGTAACC ATATTCGATT GGATGATTTG CTAAAAATGA |
| | ATGATCTCGA |
| 651 | TGAATATAAA GCCCGGCGCC TTAAGCCTGG AGATCAGTTG |
| | CGCATACGTT |
| 701 | GA |

The PSORT algorithm predicts periplasmic (0.935).

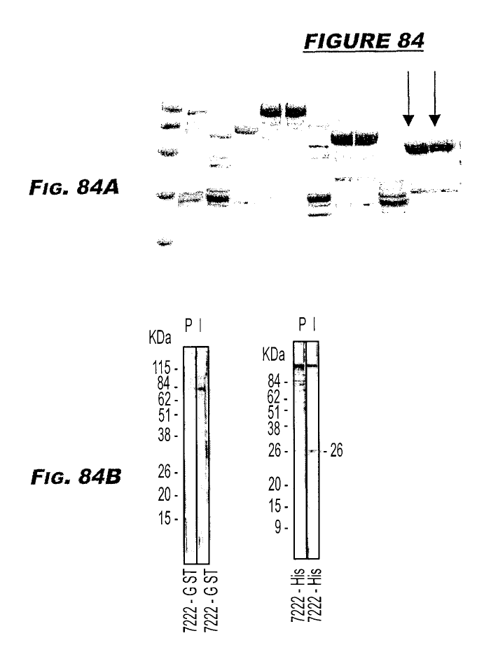

The protein was expressed in E. coli and purified as a GST-fusion product (FIG. 84A) and also in his-tagged form. The recombinant proteins were used to immunise mice, whose sera were used in a Western blot (FIG. 84B) and for FACS analysis.

These experiments show that cp7222 is a surface-exposed and immunoaccessible protein, and that it is a useful immunogen. These properties are not evident from the sequence alone.

### Example 85

The following C. pneumoniae protein (PID 4377225) was expressed <SEQ ID 169; cp7225>:

| | |
|---|---|
| 1 | MKGTPQYHFI GIGGIGMSAL AHILLDRGYE VSGSDLYESY |
| | TIESLKAKGA |
| 51 | RCFSGHDSSH VPHDAVVVYS SSIAPDNVEY LTAIQRSSRL |
| | LHRAELLSQL |
| 101 | MEGYESILVS GSHGKTGTSS LIRAIFQEAQ KDPSYAIGGL |
| | AANCLNGYSG |
| 151 | SSKIFVAEAD ESDGSLKHYT PRAVVITNID NEHLNNYAGN |
| | LDNLVQVIQD |
| 201 | FSRKVTDLNK VFYNGDCPIL KGNVQGISYG YSPECQLHIV |
| | SYNQKAWQSH |
| 251 | FSFTFLGQEY QDIELNLPGQ HNAANAAAAC GVALTFGIDI |
| | NIIRKALKKF |
| 301 | SGVHRRLERK NISESFLFLE DYAHHPVEVA HTLRSVRDAV |
| | GLRRVIAIFQ |
| 351 | PHRFSRLEEC IQTFPKAFQE ADEVILTDVY SAGESPRESI |
| | ILSDKAEQIR |
| 401 | KSSYVHCCYV PHGDIVDYLR NYIRIHDVCV SLGAGNIYTI |
| | GEALKDFNPK |
| 451 | KLSIGLVCGG KSCEHDISLL SAQHVSKYIS PEFYDVSYFI |
| | INRQGLWRTG |
| 501 | KDFPHLIEET QGDSPLSSEI ASALAKVDCL FPVLHGPFGE |
| | DGTIQGFFEI |
| 551 | LGKPYAGPSL SLAATAMDKL LTKRIASAVG VPVVPYQPLN |
| | LCFWKRNPEL |
| 601 | CIQNLIETFS FPMIVKTAHL GSSIGIFLVR DKEELQEKIS |
| | EAFLYDTDVF |
| 651 | VEESRLGSRE IEVSCIGHSS SWYCMAGPNE RCGASGFIDY |
| | QEKYGPDGID |
| 701 | CAKISFDLQL SQESLDCVRE LAERVYRAMQ GKGSARIDFF |
| | LDEEGNYWLS |
| 751 | EVNPIPGMTA ASPFLQAFVH AGWTQEQIVD HFIIDALHKF |
| | DKQQTIEQAF |
| 801 | TKEQDLVKR* |

The cp7225 nucleotide sequence <SEQ ID 170> is:

| | |
|---|---|
| 1 | ATGAAGGGAA CTCCTCAGTA TCATTTTATC GGTATCGGTG |
| | GTATAGGAAT |
| 51 | GAGCGCTTTA GCTCATATTT TCGTTGATCG TGGCTATGAG |
| | GTCTCTGGAA |
| 101 | GCGACTTATA TGAAAGCTAT ACGATCGAAA GCCTGAAAGC |
| | TAAAGGTGCG |
| 151 | AGGTGTTTCT CAGGCCATGA TTCCTCCCAT GTTCCTCATG |
| | ATGCCGTCGT |
| 201 | TGTTTATAGC TCAAGTATAG CCCCTGATAA TGTAGAGTAT |
| | CTTACCGCTA |
| 251 | TTCAAAGATC ATCACGTCTT CTTCATAGAG CAGAGCTCTT |
| | GAGTCAGCTT |
| 301 | ATGGAGGGTT ATGAAAGCAT TCTGGTTTCA GGAAGCCATG |
| | GGAAGACAGG |
| 351 | GACCTCATCT CTAATTCGAG CGATTTTCCA GGAAGCTCAG |
| | AAAGATCCCT |
| 401 | CCTATGCTAT TGGAGGACTC GCTGCAAACT GCCTGAATGG |
| | GTATTCTGGA |
| 451 | TCATCGAAAA TCTTCGTTGC CGAAGCCGAT GAAAGTGATG |
| | GGTCTTTAAA |
| 501 | GCACTACACT CCCCGTGCAG TAGTCATTAC AAATATAGAT |
| | AATGAACATT |
| 551 | TGAATAATTA CGCTGGGAAT CTTGATAACC TGGTTCAGGT |
| | AATCCAGGAC |
| 601 | TTCTCTAGAA AAGTAACAGA TCTCAATAAG GTATTCTATA |
| | ACGGGGATTG |
| 651 | TCCTATTTTG AAAGGAAATG TCCAAGGGAT TTCTTATGGA |
| | TATTCACCAG |
| 701 | AATGTCAATT GCATATCGTT TCCTATAATC AAAAGGCATG |
| | GCAATCTCAC |
| 751 | TTTTCCTTTA CCTTTTTAGG CCAGGAGTAT CAAGACATTG |
| | AGCTCAATCT |
| 801 | CCCTGGACAA CATAACGCTG CAAATGCAGC AGCAGCCTGT |
| | GGAGTTGCTC |
| 851 | TTACCTTTGG CATAGACATA AACATCATTC GAAAAGCTCT |
| | CAAAAAATTC |
| 901 | TCGGGAGTTC ATCGACGTCT AGAAAGAAAA AATATATCCG |
| | AAAGCTTTCT |
| 951 | TTTCTTAGAA GATTATGCTC ATCATCCTGT AGAGGTTGCA |
| | CATACCCTGC |
| 1001 | GCTCTGTGCG TGATGCTGTG GGTTTGCGAA GAGTCATCGC |
| | AATTTTTCAA |
| 1051 | CCACATCGAT TCTCTCGTTT AGAAGAGTGC TTACAAACCT |
| | TCCCCAAAGC |
| 1101 | TTTCCAAGAA GCTGATGAAG TCATACTTAC AGATGTCTAT |
| | AGTGCCGGAG |
| 1151 | AAAGTCCTAG AGAGTCTATC ATTCTTTCCG ACCTTGCGGA |
| | ACAGATTCGT |
| 1201 | AAGTCTTCTT ATGTCCATTG TTGTTATGTT CCCCATGGAG |
| | ACATCGTAGA |
| 1251 | TTATCTACGA AACTACATTC GCATTCATGA TGTCTGTGTT |
| | TCTCTAGGAG |
| 1301 | CTGGAAATAT CTATACTATT GGAGAGGCTT TAAAAGACTT |
| | TAACCCTAAA |
| 1351 | AAATTATCCA TAGGACTCGT CTGTGGAGGG AAATCTTGCG |
| | AACACGATAT |
| 1401 | TTCTCTACTT TCTGCTCAAC ATGTCTCTAA ATATATTTCT |
| | CCTGAATTCT |
| 1451 | ATGATGTGAG TTACTTCATC ATAAATCGTC AGGGCTTATG |
| | GAGAACAGGA |
| 1501 | AAGGATTTTC CTCATCTTAT TGAAGAGACT CAAGGGGATT |
| | CGCCACTTTC |
| 1551 | TTCTGAAATC GCTTCAGCTT TAGCAAAAGT CGACTGTTTG |
| | TTTCCCGTGC |
| 1601 | TCCATGGCCC ATTTGGAGAG GATGGTACGA TCCAGGGATT |
| | TTTTGAAATC |
| 1651 | TTAGGAAAAC CTTATGCCGG ACCCTCACTA TCTTTAGCAG |
| | CAACTGCAAT |
| 1701 | GGATAAGCTG TTAACAAAAC GAATTGCATC AGCAGTGGGT |
| | GTTCCTGTAG |
| 1751 | TCCCTTACCA ACCTTTAAAT CTCTGTTTCT GGAAACGCAA |
| | TCCAGAACTA |
| 1801 | TGTATTCAGA ATCTTATAGA GACATTTTCT TTCCCTATGA |
| | TTGTAAAAAC |
| 1851 | TGCACATTTG GGATCTAGTA TTGGGATATT TTTAGTCCGT |
| | GATAAAGAGG |
| 1901 | AATTACAAGA AAAGATCTCA GAAGCATTTC TATATGACAC |
| | GGATGTGTTT |
| 1951 | GTGGAGGAAA GTCGCTTAGG GTCTCGTGAA ATCGAAGTGT |
| | CCTGTATCGG |
| 2001 | CCATTCTTCT AGCTGGTATT GTATGGCAGG GCCTAATGAA |
| | CGCTGTGGTG |
| 2051 | CTAGTGGGTT TATTGATTAT CAAGAGAAAT ATGGATTTGA |
| | TGGCATAGAT |
| 2101 | TGCGCAAAGA TCTCTTTTGA TTTACAGCTC TCACAAGAAT |
| | CTTTAGATTG |
| 2151 | TGTTAGAGAA CTTGCAGAGC GTGTCTACCG AGCAATGCAA |
| | GGAAAAGGTT |
| 2201 | CAGCTCGAAT AGATTTTTTC TTGGATGAAG AGGGGAATTA |
| | TTGGTTGTCA |
| 2251 | GAGGTCAATC CTATTCCAGG AATGACAGCA GCTAGCCCAT |
| | TTTTACAAGC |
| 2301 | TTTTGTTCAC GCAGGATGGA CGCAAGAACA AATTGTAGAT |
| | CACTTTATTA |
| 2351 | TAGATGCTCT ACATAAGTTT GATAAGCAGC AGACTATCGA |
| | ACAGGCATTC |
| 2401 | ACTAAAGAAC AAGATTTAGT TAAAAGATAA |

The PSORT algorithm predicts inner membrane (0.16).

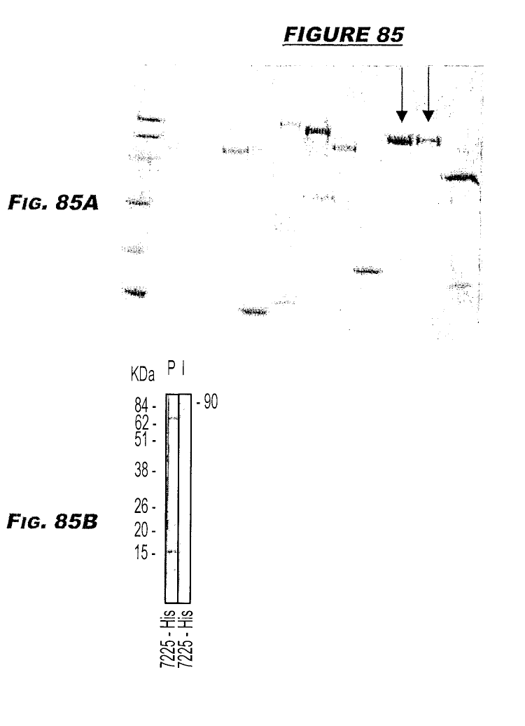

The protein was expressed in E. coli and purified as a his-tag product (FIG. 85A). The recombinant protein was used to immunise mice, whose sera were used in a Western blot (FIG. 85B) and for FACS analysis.

These experiments show that cp7225 is a surface-exposed and immunoaccessible protein, and that it is a useful immunogen. These properties are not evident from the sequence alone.

### Example 86

The following C. pneumoniae protein (PID 4377248) was expressed <SEQ ID 171; cp7248>:

| | |
|---|---|
| 1 | MKFWLQGCAF VGCLLLTLPC CAARRRASGE NLQQTRPIAA |
| | ANLQWESYAE |
| 51 | ALEHSKQDHK PICKFFTGSD WCMWCIKMQD QILQSSEFKH |
| | FAGVHLHMVE |
| 101 | VDFPQKNHQP EEQRQKNQEL KAQYKVTGFP ELVFIDAEGK |
| | QLARMGFEPG |
| 151 | GGAAYVSKVK SALKLR* |

A predicted signal peptide is highlighted.

The cp7248 nucleotide sequence <SEQ ID 172> is:

| | |
|---|---|
| 1 | ATGAAATTTT GGTTGCAAGG ATGTGCTTTT GTCGGTTGTC |
| | TGCTATTGAC |
| 51 | TTTACCTTGT TGTGCTGCAC GAAGACGTGC TTCTGGAGAA |
| | AATTTGCAAC |
| 101 | AAACTCGTCC TATAGCAGCT GCAAATCTAC AATGGGAGAG |
| | CTATGCAGAA |
| 151 | GCTCTTGAAC ATTCTAAACA AGATCACAAA CCTATTTGTC |
| | TTTTCTTTAC |
| 201 | AGGATCAGAC TGGTGTATGT GGTGCATAAA AATGCAAGAC |
| | CAGATTTTGC |
| 251 | AAAGCTCTGA GTTTAAGCAT TTTGCGGGTG TGCATCTGCA |
| | TATGGTTGAA |
| 301 | GTTGATTTCC CCCAAAAGAA TCATCAACCT GAAGAGCAGC |
| | GCCAAAAAAA |
| 351 | TCAAGAACTG AAAGCTCAAT ATAAAGTTAC AGGATTCCCC |
| | GAACTGGTCT |
| 401 | TCATAGATGC AGAAGGAAAA CAGCTTGCTC GCATGGGATT |
| | TGAGCCTGGT |
| 451 | GGTGGAGCTG CTTACGTAAG CAAGGTGAAG TCTGCTCTTA |
| | AACTACGTTA |
| 501 | A |

The PSORT algorithm predicts periplasmic (0.932).

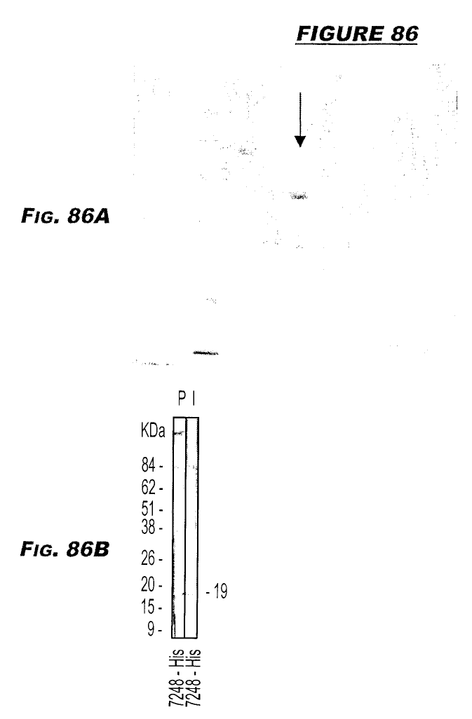

The protein was expressed in E. coli and purified as a GST-fusion product (FIG. 86A) and also in his-tagged form. The recombinant proteins were used to immunise mice, whose sera were used in a Western blot (FIG. 86B) and for FACS analysis.

The cp7248 protein was also identified in the 2D-PAGE experiment.

These experiments show that cp7248 is a surface-exposed and immunoaccessible protein, and that it is a useful immunogen. These properties are not evident from the sequence alone.

### Example 87

The following C. pneumoniae protein (PID 4377249) was expressed <SEQ ID 173; cp7249>:

| | |
|---|---|
| 1 | MIPSPTPINF RDDTILETDP KPSLIMFSSK KTEIASERRK |
| | AHPTLFKVLG |
| 51 | TIWNIVKFII SIILFLPLAL LWVLKKTCQF FILPSSIISQ |
| | SMSKTAVAIR |
| 101 | RMTFLSHIKQ LLSLKEISAA DRVVIQYDDL VVDSLAIKIP |
| | HALPHRWILY |
| 151 | SQGNSGLMEN LFDRGDSSLH QLAKATGSNL LVFNYPGIMS |
| | SKGEAKRENL |
| 201 | VKSYQACVRY LRDEEIGPKA NQIIAFGYSL GTSVQAAALD |
| | REVIDGSDGT |
| 251 | SWIVVKDRGP RSLADVANQI CKPIASAIIK LVGWNIDSVK |
| | PSERLRCPEI |
| 301 | FIYNSNHDQE LISDGLFERE NCVATPFLEL PEVKTSGTKI |
| | PIPERDLLHL |
| 351 | NPLSPNVVDR LAAVISNYLD SENRKSQQPD * |

The cp7249 nucleotide sequence <SEQ ID 174> is:

| | |
|---|---|
| 1 | ATGATCCCAT CCCCTACCCC AATAAACTTT CGTGATGATA |
| | CGATTCTAGA |
| 51 | GACGGATCCA AAGCCGTCTT TAATCATGTT CTCTTCAAAA |
| | AAAACAGAGA |
| 101 | TAGCTTCTGA AAGACGGAAG GCCCATCCCA CCTTATTTAA |
| | AGTTCTAGGA |
| 151 | ACGATTTGGA ATATTGTGAA GTTTATTATC TCAATCATTC |
| | TGTTCCTTCC |
| 201 | CTTAGCGTTA TTGTGGGTAC TCAAGAAAAC CTGTCAGTTT |
| | TTCATTCTCC |
| 251 | CATCTTCTAT CATATCTCAG AGCATGTCAA AAACAGCTGT |
| | GGCAATTCGG |
| 301 | CGAATGACCT TTCTGTCCCA TATTAAACAA CTCCTAAGCC |
| | TTAAGGAAAT |
| 351 | CTCAGCTGCC GATCGTGTGG TTATACAATA TGACCATTTG |
| | GTGGTTGATA |
| 401 | GCTTAGCTAT AAAGATACCT CATGCTCTTC CCCACAGGTG |
| | GATTCTTTAT |
| 451 | TCTCAAGGAA ACTCTGGATT GATGGAAAAC CTGTTCCATC |
| | GGGGCGATTC |
| 501 | CTCTCTACAC CAGCTAGCCA AAGCAACCGG CTCGAATCTT |
| | CTTGTGTTCA |
| 551 | ACTATCCTGG AATTATGTCC AGCAAAGGAG AAGCGAAACG |
| | AGAAAATCTG |
| 601 | GTTAAATCGT ATCAGGCATG CGTACGCTAC CTACGAGATG |
| | AAGAGACAGG |
| 651 | TCCTAAAGCC AATCAAATCA TAGCTTTCGG ATACTCTTTG |
| | GGAACTAGTG |
| 701 | TCCAAGCTGC TGCTCTAGAT CGTGAGGTCA CTGATGGCAG |
| | TGATGGAACT |
| 751 | TCATGGATTG TTGTAAAAGA TCGGGGCCCT CGCTCTCTAG |
| | CAGATGTCGC |
| 801 | GAGTCAAATT TGTAAGCCCA TAGCTTCCGC GATTATAAAA |
| | CTCGTTGGTT |
| 851 | GGAACATAGA CTCTGTGAAA CCTAGCGAAA GATTGCGTTG |
| | TCCCGAAATT |
| 901 | TTCATTTACA ACTCTAATCA TGATCAAGAA CTCATTAGCG |
| | ACGGCCTCTT |
| 951 | CGAAAGAGAA AATTGCGTAG CAACACCTTT TCTAGAGCTT |
| | CCTGAAGTAA |
| 1001 | AAACCTCGGG GACTAAAATT CCTATACCCG AAAGGGATCT |
| | TCTCCATCTA |
| 1051 | AATCCTCTCA GTCCAAATGT AGTAGACAGA TTAGCAGCAG |
| | TGATCTCTAA |
| 1101 | TTATTTAGAT TCTGAAAACA GAAAGTCTCA GCAACCTGAT |
| | TAA |

The PSORT algorithm predicts inner membrane (0.571).

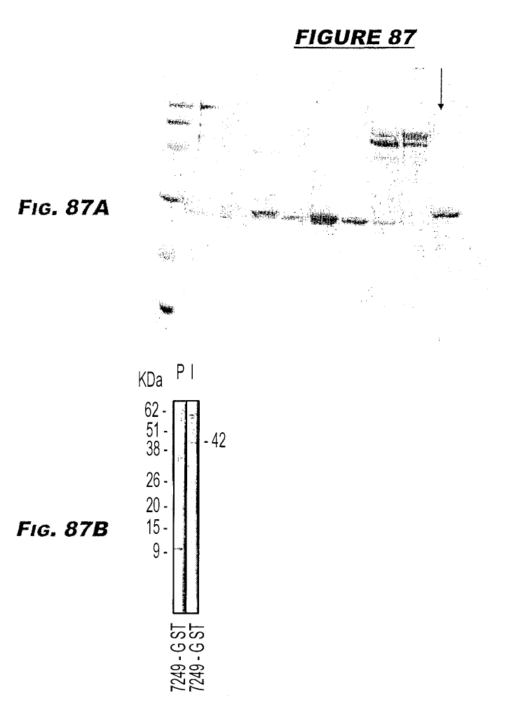

The protein was expressed in E. coli and purified as a GST-fusion product (FIG. 87A). The recombinant protein was used to immunise mice, whose sera were used in a Western blot (FIG. 87B) and for FACS analysis.

These experiments show that cp7249 is a surface-exposed and immunoaccessible protein, and that it is a useful immunogen. These properties are not evident from the sequence alone.

### Example 88

The following C. pneumoniae protein (PID 4377261) was expressed <SEQ ID 175; cp7261>:

| | |
|---|---|
| 1 | MLPISILLFY VILGCLSAYI ADKKKRNVIG WFFAGAFFGF |
| | IGLVVLLLLP |
| 51 | SRRNALEKPQ NDPFDNSDLF DDLKKSLAGN DEIPSSGDLQ |
| | EIVIDTEKWF |
| 101 | YLNKDRENVG PISFEELVVL LKGKTYPEFE WVWKKGMKDW |
| | QRVKDVPSLQ |
| 151 | QALKEASK* |

The cp7261 nucleotide sequence <SEQ ID 176> is:

| | |
|---|---|
| 1 | ATGCTCCCTA TTTCGATTTT ATTATTTTAT GTGATTCTAG |
| | GTTGTCTATC |
| 51 | TGCCTACATA GCAGATAAGA AAAAACGAAA TGTTATTGGC |
| | TGGTTTTTTG |
| 101 | CAGGAGCATT TTTTGGATTT ATTGGTCTAG TTGTCCTTCT |
| | TCTTCTTCCT |
| 151 | TCTCGTCGAA ACGCTTTAGA AAAGCCACAA AACGATCCTT |
| | TTGATAACTC |
| 201 | CGATCTTTTT GATGATTTGA AAAAAAGTTT AGCAGGTAAT |
| | GACGAGATAC |
| 251 | CCTCATCGGG AGATCTTCAA GAAATCGTTA TCGATACAGA |
| | GAAGTGGTTT |
| 301 | TATTTAAATA AAGATAGAGA AAACGTAGGT CCGATATCTT |
| | TTGAGGAGTT |
| 351 | GGTCGTACTT TTAAAGGGAA AAACGTATCC AGAAGAAATT |
| | TGGGTATGGA |
| 401 | AAAAGGGAAT GAAAGATTGG CAACGAGTGA AGGATGTTCC |
| | ATCACTACAA |
| 451 | CAGGCTTTGA AAGAAGCATC AAAATAA |

The PSORT algorithm predicts inner membrane (0.848).

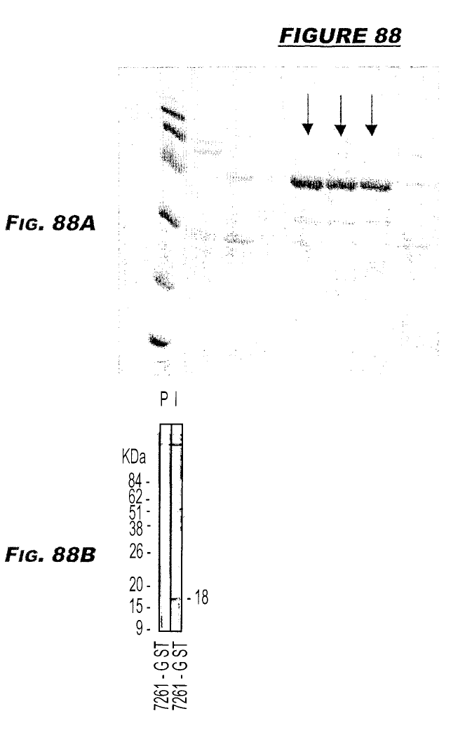

The protein was expressed in E. coli and purified as a GST-fusion product (FIG. 88A). The recombinant protein was used to immunise mice, whose sera were used in a Western blot (FIG. 88B) and for FACS analysis.

These experiments show that cp7261 is a surface-exposed and immunoaccessible protein, and that it is a useful immunogen. These properties are not evident from the sequence alone.

### Example 89

The following C. pneumoniae protein (PID 4377305) was expressed <SEQ ID 177; cp7305>:

| | |
|---|---|
| 1 | MEVYSFHPAV RTSFQHRVMA ALDAWFFLGG HRLKVVSLDS |
| | CNSGWAYQEL |
| 51 | VSISTTEKVL KLLSYLLVPI VIIALLIRCL LHSNFRIDVE |
| | KERWLKIREL |
| 101 | GIDIESCKLP SSYVNQVSSF IWFEKDKSKR PRIDVDYHTL |
| | HSKDWVVFPI |
| 151 | VFQKIPKTSR FSYWFSQKET RKRDYVRNML DHVIGYLTSE |
| | GGEWLQYISK |
| 201 | ISYQSATSLD PERVLQYCLT DNQELQGEVQ RLLNEESATK |
| | SSGDKEVLLS |
| 251 | HVSDIICQCW WPKFLEVIQS RAFIFELVEE VSGKLNLDFL |
| | CLEKANTLDQ |
| 301 | ELRNSLLRAV VHHGSEGVDI KKVGAGLIIY TEAIQLQIPF |
| | SRS* |

The cp7305 nucleotide sequence <SEQ ID 178> is:

| | |
|---|---|
| 1 | ATGGAAGTTT ATAGTTTTCA CCCTGCGGTA AGGACTTCGT |
| | TTCAGCACCG |
| 51 | TGTAATGGCA GCACTAGATG CTTGGTTTTT TCTAGGAGGG |
| | CACCGTTTAA |
| 101 | AGGTAGTTTC TCTAGATAGT TGTAACTCAG GTTGGGCGTA |
| | TCAAGAACTT |
| 151 | GTGTCTATTT CAACGACAGA AAAAGTCTTG AAACTACTCT |
| | CTTACCTACT |
| 201 | CGTACCGATT GTCATAATAG CTCTGTTAAT TCGTTGTCTT |
| | TTACATAGCA |
| 251 | ATTTTAGGAT AGACGTAGAG AAGGAACGTT GGTTAAAAAT |
| | AAGGGAGTTA |
| 301 | GGAATTGATA TAGAAAGCTG CAAACTCCCC AGTTCTTATG |
| | TAAACCAGGT |
| 351 | TTCCTCGTTT ATTTGGTTTG AAAAAGATAA ATCCAAACGG |
| | CCACGTATTG |
| 401 | ATGTAGATTA TCATACGCTA CATAGCAAAG ACTGGGTAGT |
| | TTTCCCTATC |
| 451 | GTTTTTCAGA AAATTCCAAA GACCTCGCGT TTCAGTTATT |
| | GGTTCTCACA |
| 501 | AAAAGAAACA AGGAAGAGGG ATTATGTGAG AAATATGCTG |
| | GACCACGTCA |
| 551 | TTGGTTATCT AACGTCAGAA GGTGGGGAGT GGTTGCAGTA |
| | TATATCGAAA |
| 601 | ACCTCTTATC AAAGCGCTAC TTCCTTGGAT CCTGAAAGAG |
| | TTCTTCAATA |
| 651 | TTGCTTAACT GATAACCAGG AGCTCCAGGG AGAAGTGCAA |
| | CGTTTGCTTA |
| 701 | ATGAGGAGAG TGCGACCAAA AGCTCTGGGG ATAAGGAAGT |
| | TTTGTTAAGT |
| 751 | CATGTATCTG ACATTATTTG CCAGTGTTGG TGGCCAAAGT |
| | TTCTTGAAGT |
| 801 | TATACAATCT CCGGCCTTTA TTGAAGAATT AGTAGAAGAA |
| | GTGAGTGGTA |
| 851 | AACTTAATTT AGATTTTTTA TGCCTAGAAA AGGCTAATAC |
| | ATTAGATCAG |
| 901 | GAGTTGAGAA ACAGTCTTCT AAGAGCAGTC GTACACCACG |
| | GTTCTGAAGG |
| 951 | AGTTGATATT AAGAAAGTTG GTGCCGGCCT CATTATTTAT |
| | ACGGAAGCTA |
| 1001 | TTCAATTACA GATTCCCTTC TCAAGGAGTT AA |

The PSORT algorithm predicts inner membrane (0.508).

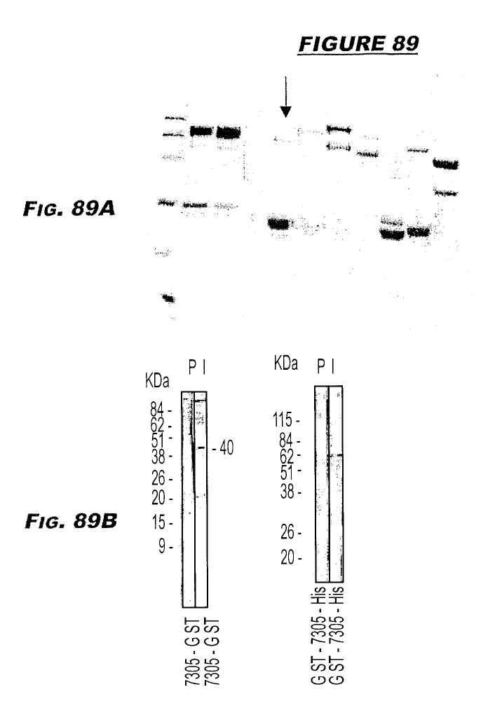

The protein was expressed in E. coli and purified as a GST-fusion product (FIG. 89A) and also as a double GST/his fusion. The recombinant proteins were used to immunise mice, whose sera were used in a Western blot (FIG. 89B) and for FACS analysis.

These experiments show that cp7305 is a surface-exposed and immunoaccessible protein, and that it is a useful immunogen. These properties are not evident from the sequence alone.

### Example 90

The following C. pneumoniae protein (PID 4377347) was expressed <SEQ ID 179; cp7347>:

| | |
|---|---|
| 1 | MKKGKLGAIV FGLLFTSSVA GFSKDLTKDN AYQDLNVIEH |
| | LISKLYAPLP |
| 51 | WKELLFGWDL SQQTQQARLQ LVLEEKPTTN YCQKVLSNYV |
| | RSLNDYHAGI |
| 101 | TFYRTESAYI PYVLKLSEDG HVFVVDVQTS QGDIYLGDEI |
| | LEVDGMGIRE |
| 151 | AIESLRFGRG SATDYSAAVR SLTSRSAAFG DAVPSGIAML |
| | KLRRPSGLIR |
| 201 | STPVRWRYTP EHIGDFSLVA PLIPEHKPQL PTQSCVLFRS |
| | GVNSQSSSSS |
| 251 | LFSSYMVPYF WEELRVQNKQ RFDSNHHIGS RNGFLPTFGP |
| | ILWEQDKGPY |
| 301 | RSYIFKAKDS QGNPHRIGFL RISSYVWTDL EGLEEDHKDS |
| | PWELFGEIID |
| 351 | HLEHETDALI IDQTHNPGGS VFYLYSLLSM LTDHPLDTPK |
| | HRMIFTQDFV |
| 401 | SSALHWQDLL EDVFTDWQAV AVLGETMEGY CMDMHAVASL |
| | QNFSQSVLSS |
| 451 | WVSGDINLSK PMPLLGFAQV RPHPKHQYTK PLFMLIDEDD |
| | FSCGDLAPAI |
| 501 | LKDNGRATLI GKPTAGAGGF VFQVTFPNRS GIKGLSLTGS |
| | LAVRKDGEFI |
| 551 | ENLGVAPHID LGFTSRDLQT SRFTDYVEAV KTIVLTSLSE |
| | NAKKSEEQTS |
| 601 | PQETPEVIRV SYPTTTSAS* |

A predicted signal peptide is highlighted.

The cp7347 nucleotide sequence <SEQ ID 180> is:

| | |
|---|---|
| 1 | ATGAAAAAAG GGAAATTAGG AGCCATAGTT TTTGGCCTTC |
| | TATTTACAAG |
| 51 | TAGTGTTGCT GGTTTTTCTA AGGATTTGAC TAAAGACAAC |
| | GCTTATCAAG |
| 101 | ATTTAAATGT CATAGAGCAT TTAATATCGT TAAAATATGC |
| | TCCTTTACCA |
| 151 | TGGAAGGAAC TATTATTTGG TTGGGATTTA TCTCAGCAAA |
| | CACAGCAAGC |
| 201 | TCGCTTGCAA CTGGTCTTAG AAGAAAAACC AACAACCAAC |
| | TACTGCCAGA |
| 251 | AGGTACTCTC TAACTACGTG AGATCATTAA ACGATTATCA |
| | TGCAGGGATT |
| 301 | ACGTTTTATC GTACTGAAAG TGCGTATATC CCTTACGTAT |
| | TGAAGTTAAG |
| 351 | TGAAGATGGT CATGTCTTTG TAGTCGACGT ACAGACTAGC |
| | CAAGGGGATA |
| 401 | TTTACTTAGG GGATGAAATC CTTGAAGTAG ATGGAATGGG |
| | GATTCGTGAG |
| 451 | GCTATCGAAA GCCTTCGCTT TGGACGAGGG AGTGCCACAG |
| | ACTATTCTGC |
| 501 | TGCAGTTCGT TCCTTGACAT CGCGTTCCGC CGCTTTTGGA |
| | GATGCGGTTC |
| 551 | CTTCAGGAAT TGCCATGTTG AAACTTCGCC GACCCAGTGG |
| | TTTGATCCGT |
| 601 | TCGACACCGG TCCGTTGGCG TTATACTCCA GAGCATATCG |
| | GAGATTTTTC |
| 651 | TTTAGTTGCT CCTTTGATTC CTGAACATAA ACCTCAATTA |
| | CCTACACAAA |
| 701 | GTTGTGTGCT ATTCCGTTCC GGGGTAAATT CACAGTCTTC |
| | TAGTAGCTCT |
| 751 | TTATTCAGTT CCTACATGGT GCCTTATTTC TGGGAAGAAT |
| | TGCGGGTTCA |
| 801 | AAATAAGCAG CGTTTTGACA GTAATCACCA TATAGGGAGC |
| | CGTAATGGAT |
| 851 | TTTTACCTAC GTTTGGTCCT ATTCTTTGGG AACAAGACAA |
| | GGGGCCCTAT |
| 901 | CGTTCCTATA TCTTTAAAGC AAAAGATTCT CAGGGCAATC |
| | CCCATCGCAT |
| 951 | AGGATTTTTA AGAATTTCTT CTTATGTTTG GACTGATTTA |
| | GAAGGACTTG |
| 1001 | AAGAGGATCA TAAGGATAGT CCTTGGGAGC TCTTTGGAGA |
| | GATCATCGAT |
| 1051 | CATTTGGAAA AAGAGACTGA TGCTTTGATT ATTGATCAGA |
| | CCCATAATCC |
| 1101 | TGGAGGCAGT GTTTTCTATC TCTATTCGTT ACTATCTATG |
| | TTAACAGATC |
| 1151 | ATCCTTTAGA TACTCCTAAA CATAGAATGA TTTTCACTCA |
| | GGATGAAGTC |
| 1201 | AGCTCGGCTT TGCACTGGCA AGATCTACTA GAAGATGTCT |
| | TCACAGATGA |
| 1251 | GCAGGCAGTT GCCGTGCTAG GGGAAACTAT GGAAGGATAT |
| | TGCATGGATA |
| 1301 | TGCATGCTGT AGCCTCTCTT CAAAACTTCT CTCAGAGTGT |
| | CCTTTCTTCC |
| 1351 | TGGGTTTCAG GTGATATTAA CCTTTCAAAA CCTATGCCTT |
| | TGCTAGGATT |
| 1401 | TGCACAGGTT CGACCTCATC CTAAACATCA ATATACTAAA |
| | CCTTTGTTTA |
| 1451 | TGTTGATAGA CGAGGATGAC TTCTCTTGTG GAGATTTAGC |
| | GCCTGCAATT |
| 1501 | TTGAAGGATA ATGGCCGCGC TACTCTCATT GGAAAGCCAA |
| | CAGCAGGAGC |
| 1551 | TGGAGGTTTT GTATTCCAAG TCACTTTCCC TAACCGTTCT |
| | GGAATTAAAG |
| 1601 | GTCTTTCTTT AACAGGATCT TTAGCTGTTA GGAAAGATGG |
| | TGAGTTTATT |
| 1651 | GAAAACTTAG GAGTGGCTCC TCATATTGAT TTAGGATTTA |
| | CCTCCAGGGA |
| 1701 | TTTGCAAACT TCCAGGTTTA CTGATTACGT TGAGGCAGTG |
| | AAAACTATAG |
| 1751 | TTTTAACTTC TTTGTCTGAG AACGCTAAGA AGAGTGAAGA |
| | GCAGACTTCT |
| 1801 | CCGCAAGAGA CGCCTGAAGT TATTCGAGTC TCTTATCCCA |
| | CAACGACTTC |
| 1851 | TGCTTCGTAA |

The PSORT algorithm predicts periplasmic space (0.2497).

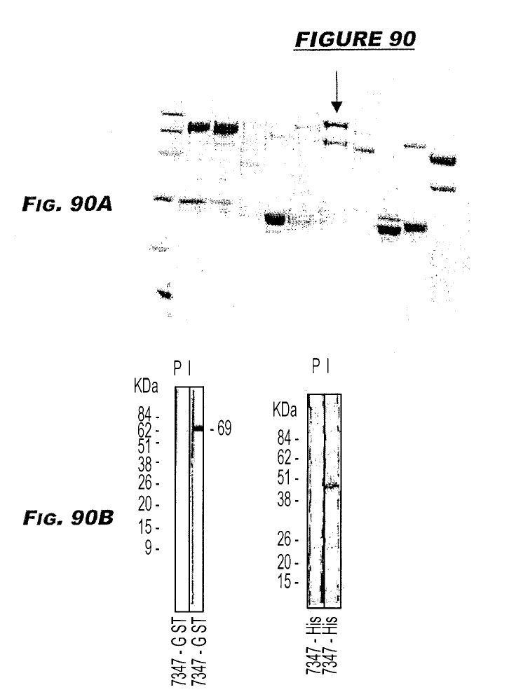

The protein was expressed in E. coli and purified as a GST-fusion product (FIG. 90A) and also in his-tagged form. The recombinant proteins were used to immunise mice, whose sera were used in a Western blot (FIG. 90B) and for FACS analysis.

These experiments show that cp7347 is a surface-exposed and immunoaccessible protein, and that it is a useful immunogen. These properties are not evident from the sequence alone.

### Example 91

The following C. pneumoniae protein (PID 4377353) was expressed <SEQ ID 181; cp7353>:

| | |
|---|---|
| 1 | MNMPVPSAVP SANITLKEDS STVSTASGIL KTATGEVLVS |
| | CTALEGSSST |
| 51 | CALISLALGQ IILATQQELL LQSTNVHQLL FLPPEVVELE |
| | IQVVDLLVQL |
| 101 | EHAETITSEP QETQTQSRSE QTLPQQSSSK QSALSPRSLK |
| | PEISDSKQQQ |
| 151 | ALQTPKDSAV RKHSEAPSPE TQARASLSQA SSSSQRSLPP |
| | QESAPERTLL |
| 201 | EQQKASSFSP LSQFSAEKQK EALTTSKSHE LYKERDQDRQ |
| | QREQHDRKHD |
| 251 | QEEDAESKKK KKKRGLGVEA VAEEPGENLD IAALIFSDQM |
| | RPPAEETSKK |
| 301 | ETTFKKKLPS PMSVFSRFIP SKNPLSVGSS IHGPIQTPKV |
| | ENVFLRFMKL |
| 351 | MARILGQAEA EANELYMRVK QRTDDVDTLT VLISKINNEK |
| | KDIDWSENEE |
| 401 | MKALLNRAKE IGVTIDKEKY TWTEEEKRLL KENVQMRKEN |
| | MEKITQMERT |
| 451 | DMQRHLQEIS QCHQARSNVL KLLKELMDTF IYNLRP* |

The cp7353 nucleotide sequence <SEQ ID 182> is:

| | |
|---|---|
| 1 | ATGAATATGC CTGTTCCTTC TGCAGTTCCC TCTGCAAATA |
| | TAACTCTAAA |
| 51 | AGAAGACAGC TCAACAGTTT CCACAGCCTC TGGAATATTA |
| | AAGACTGCAA |
| 101 | CAGGTGAAGT CTTAGTCTCT TGTACAGCGC TAGAAGGAAG |
| | CTCTTCTACA |
| 151 | GATGCTTTAA TTAGCTTAGC TTTAGGACAA ATCATTCTTG |
| | CGACCCAACA |
| 201 | AGAACTGCTC TTACAAAGCA CAAATGTTCA TCAACTCCTC |
| | TTCCTCCCTC |
| 251 | CTGAAGTTGT AGAATTAGAA ATCCAAGTTG TTGACTTGCT |
| | AGTGCAATTG |
| 301 | GAACATGCAG AGACAATCAC AAGTGAACCA CAAGAAACAC |
| | AAACGCAAAG |
| 351 | TAGGAGTGAG CAGACCCTCC CTCAACAAAG CAGCAGTAAA |
| | CAATCTGCTC |
| 401 | TCTCCCCACG CTCCTTAAAA CCTGAAATTT CTGATTCTAA |
| | ACAACAGCAA |
| 451 | GCTCTTCAAA CACCAAAAGA CTCTGCTGTA AGAAAACACA |
| | GCGAAGCACC |
| 501 | GTCACCTGAG ACACAAGCTC GCGCTTCCTT ATCTCAGGCA |
| | AGCTCAAGTT |
| 551 | CTCAGAGATC CTTACCTCCG CAAGAAAGTG CGCCAGAAAG |
| | AACACTATTA |
| 601 | GAACAACAAA AAGCAAGCTC CTTCTCTCCT CTATCCCAGT |
| | TCTCTGCAGA |
| 651 | GAAACAAAAA GAGGCCCTGA CGACCTCAAA ATCTCATGAA |
| | CTCTATAAAG |
| 701 | AACGCGATCA AGATCGCCAA CAAAGAGAGC AGCACGACAG |
| | AAAGCACGAT |
| 751 | CAGGAAGAAG ACGCTGAATC TAAAAAGAAA AAGAAGAAAC |
| | GTGGTCTCGG |
| 801 | TGTAGAGGCA GTCGCTGAGG AACCCGGAGA AAATCTAGAT |
| | ATTGCCGCTT |
| 851 | TAATCTTCTC AGATCAAATG CGACCTCCTG CTGAAGAAAC |
| | TTCTAAAAAA |
| 901 | GAAACGACAT TCAAAAAGAA GCTACCTTCT CCAATGTCTG |
| | TGTTTAGCAG |
| 951 | ATTCATCCCT AGTAAGAATC CGTTATCTGT AGGCTCTTCA |
| | ATACACGGGC |
| 1001 | CTATACAAAC TCCAAAAGTA GAAAATGTGT TCTTAAGGTT |
| | CATGAAGCTC |
| 1051 | ATGGCAAGAA TCTTAGGCCA AGCCGAAGCC GAAGCTAATG |
| | AACTCTACAT |
| 1101 | GCGAGTCAAA CAACGTACCG ATGATGTAGA CACACTCACA |
| | GTCCTTATCT |
| 1151 | CTAAGATCAA TAATGAAAAG AAAGACATTG ATTGGAGTGA |
| | AAATGAAGAG |
| 1201 | ATGAAAGCTC TTTTAAATCG AGCTAAAGAG ATTGGAGTCA |
| | CTATAGACAA |
| 1251 | AGAAAAATAT ACTTGGACAG AAGAGGAAAA AAGACTTCTA |
| | AAAGAGAATG |
| 1301 | TCCAAATGCG CAAAGAGAAT ATGGAGAAAA TCACTCAAAT |
| | GGAAAGGACG |
| 1351 | GACATGCAAA GGCACCTCCA AGAGATTTCT CAATGTCATC |
| | AAGCGCGCTC |
| 1401 | TAATGTATTG AAGTTATTGA AAGAACTTAT GGACACCTTC |
| | ATTTACAACC |
| 1451 | TACGCCCCTA A |

The PSORT algorithm predicts cytoplasm (0.1308).

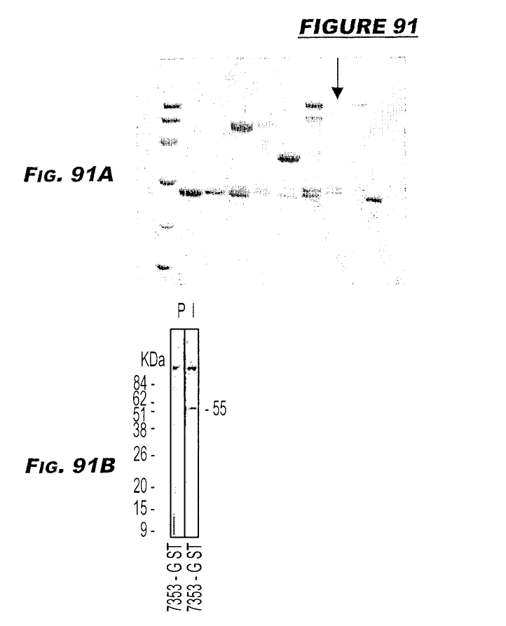

The protein was expressed in E. coli and purified as a GST-fusion product (FIG. 91A). The recombinant protein was used to immunise mice, whose sera were used in a Western blot (FIG. 91B) and for FACS analysis.

These experiments show that cp7353 is a surface-exposed and immunoaccessible protein, and that it is a useful immunogen. These properties are not evident from the sequence alone.

### Example 92

The following C. pneumoniae protein (PID 4377408) was expressed <SEQ ID 183; cp7408>:

| | |
|---|---|
| 1 | MLKIQKKRMC VSVVITVGAI VGFFNSADAA RKKKKIPIQI |
| | LYSFTKVSSY |
| 51 | LKNEDASTIF CVDVDRGLLQ HRYLGSPGWQ ETRRRQLFKS |
| | LENQSYGNER |
| 101 | LGEETLAIDI FRNKECLESE IPEQMEAILA NSSALVLGIS |
| | SFGITGIPAT |
| 151 | LHSLLRQNLS FQKRSIASES FLLKIDSAPS DASVFYKGVL |
| | FRGETAIVDA |
| 201 | LSQLFAQLDL SRKKIIFLGE DREVVQAVGS ACIGAGANFL |
| | GLVYYPAQES |
| 251 | LFSYVHPYST ATELQEAQGL QVISDEVAQL TLNALPKMN* |

The cp7408 nucleotide sequence <SEQ ID 184> is:

| | |
|---|---|
| 1 | ATGTTGAAAA TCCAGAAAAA AAGAATGTGT GTCAGCGTAG |
| | TCATCACGGT |
| 51 | AGGCGCCATA GTGGGGTTTT TCAATTCTGC AGACGCAGCA |
| | CCAAAGAAAA |
| 101 | AGAAGATCCC TATACAGATT CTCTACTCCT TTACTAAAGT |
| | CTCTTCCTAT |
| 151 | TTAAAAAACG AAGACGCAAG TACTATATTT TGCGTCGATG |
| | TGGATCGTGG |
| 201 | ACTTCTCCAG CATCGGTATT TAGGTAGTCC AGGATGGCAG |
| | GAAACCAGAC |
| 251 | GTCGGCAGTT ATTTAAATCC TTAGAAAATC AATCATACGG |
| | CAACGAACGT |
| 301 | TTAGGAGAAG AAACTCTTGC TATTGATATT TTCAGGAACA |
| | AAGAGTGCTT |
| 351 | GGAGAGCGAG ATCCCAGAGC AGATGGAAGC TATCCTTGCA |
| | AATTCCTCGG |
| 401 | CCTTGGTCTT AGGCATCTCT TCTTTTGGGA TCACAGGAAT |
| | TCCTGCGACT |
| 451 | TTGCATAGTT TGCTTCGACA GAATCTATCT TTCCAAAAAC |
| | GCTCTATAGC |
| 501 | ATCGGAGAGC TTCCTTTTAA AGATCGATAG TGCCCCCTCA |
| | GATGCCTCTG |
| 551 | TTTTTTATAA AGGCGTGCTT TTCCGCGGAG AGACTGCGAT |
| | CGTGGATGCG |
| 601 | TTAAGCCAAT TATTTGCCCA GCTCGATCTT TCTCCTAAAA |
| | AAATTATCTT |
| 651 | TCTAGGAGAA GACCCTGAGG TCGTTCAAGC TGTTGGGTCT |
| | GCTTGTATAG |
| 701 | GTTGGGGCAT GAACTTTTTA GGCCTGGTAT ACTATCCTGC |
| | TCAAGAAAGC |
| 751 | CTTTTTTCTT ATGTTCATCC TTACTCTACA GCAACGGAGC |
| | TCCAAGAAGC |
| 801 | ACAGGGTTTA CAAGTAATTT CAGATGAAGT CGCACAGCTT |
| | ACTTTAAACG |
| 851 | CTCTTCCGAA AATGAATTAA |

The PSORT algorithm predicts inner membrane (0.123).

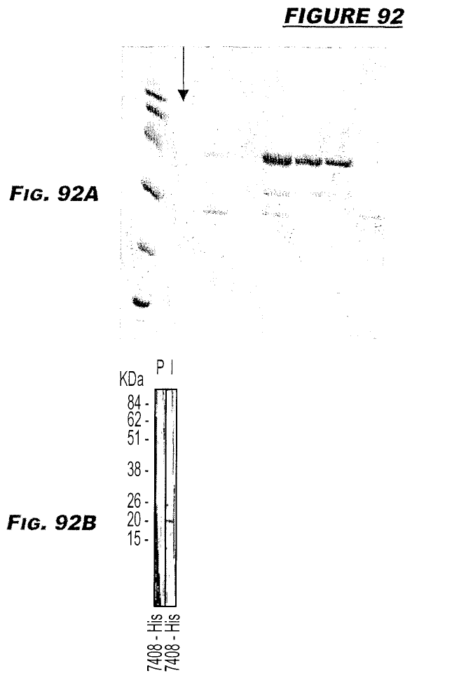

The protein was expressed in E. coli and purified as a his-tag product (FIG. 92A). The recombinant protein was used to immunise mice, whose sera were used in a Western blot (FIG. 92B) and for FACS analysis.

These experiments show that cp7408 is a surface-exposed and immunoaccessible protein, and that it is a useful immunogen. These properties are not evident from the sequence alone.

### Example 93

The following C. pneumoniae protein (PID 4376424) was expressed <SEQ ID 185; cp6424>:

| | |
|---|---|
| 1 | MMHNIVVLSE EPGRSAFLGR TAFFPNAYPI AQGGVGIPST |
| | IGNLFTIWYC |
| 51 | FYFYPAATPQ SDHPDGCGFI LLERLKELGA GFFYCDLRES |
| | NTTGETLFFE |
| 101 | GSNKGVLKNH LFIRDE* |

The cp6424 nucleotide sequence <SEQ ID 186> is:

| | |
|---|---|
| 1 | ATGATGCACA ATATTGTTGT TCTTAGTGAG GAACCTGGAC |
| | GAAGCGCTTT |
| 51 | TCTTGGTAGG ACGGCATTTT TCCCTAATAA GTATCCAATA |
| | GCTCAGGGTG |
| 101 | GTGTTGGAAT ACCATCTACA ATAGGCAATC TCTTTACTAT |
| | ATGGTACTGT |
| 151 | TTCTATTTTT AGAGAGCTGC AACTCCACAA TCTGATCATC |
| | CTGACGGATG |
| 201 | TGGCTTTATT CTACTAGAAA GGCTTAAGGA GCTCGGTGCA |
| | GGGTTCTTTT |
| 251 | ATTGTGATCT TCGTGAGTCC AATACCACTG GCTTTACTCT |
| | TTTTTTTGAA |
| 301 | GGCTCCAATA AAGGTGTGTT AAAGAATCAC TTGTTTATTA |
| | GAGATGAGTA |
| 351 | A |

The PSORT algorithm predicts cytoplasm (0.2502).

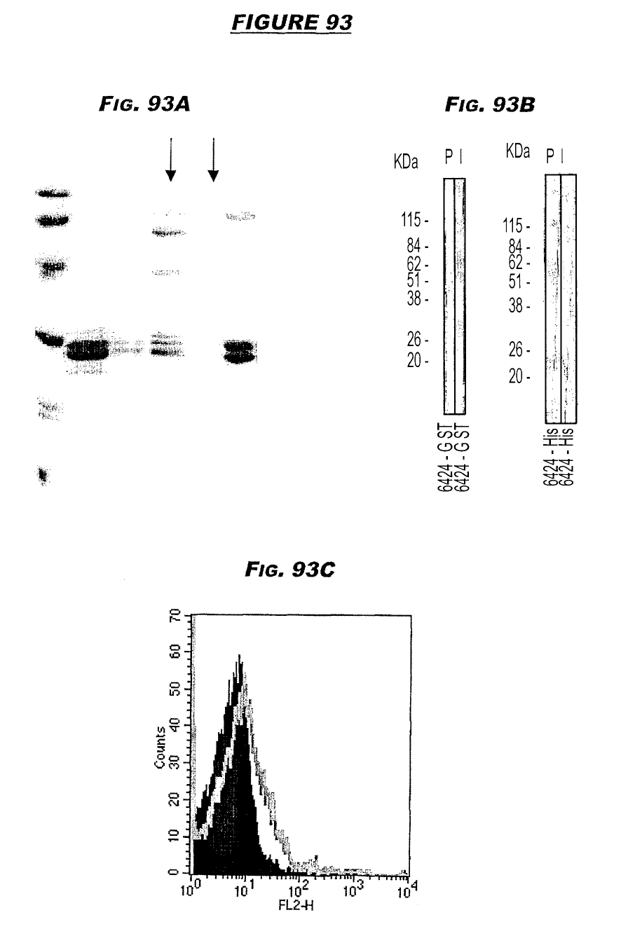

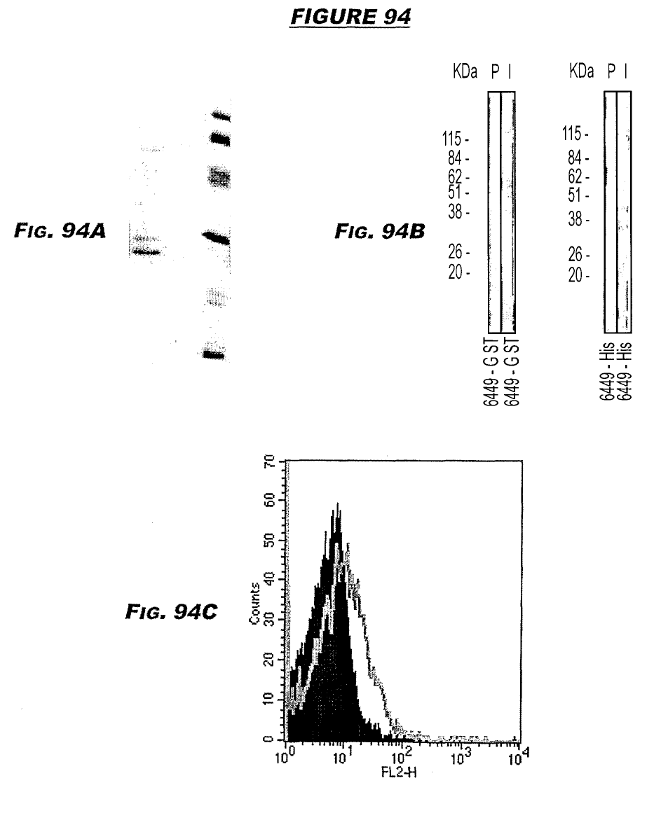

The protein was expressed in E. coli and purified as a GST-fusion product (FIG. 93A) and also in his-tagged form. The recombinant proteins were used to immunise mice, whose sera were used in Western blots (FIG. 93B) and for FACS analyses (FIG. 93C; GST-fusion).

These experiments show that cp6424 is a surface-exposed and immunoaccessible protein, and that it is a useful immunogen. These properties are not evident from the sequence alone.

### Example 94

The following C. pneumoniae protein (PID 4376449) was expressed <SEQ ID 187; cp6449>:

| | |
|---|---|
| 1 | VASETYPSQI LHAQREVRDA YFNQADCHPA RANQILEAKK |
| | ICLLDVYHTN |
| 51 | HYSVFTFCVD NYPNLRFTFV SSKNNEMNGL SNPLDNVLVE |
| | AMVRRTHARN |
| 101 | LLAACKIRNI EVPRVVGLDL RSGILISKLE LKQPQFQSLT |
| | EDFVNHSTNQ |
| 151 | EEARVHQKHV LLISLILLCK QAVLESFQEK KRSS* |

The cp6449 nucleotide sequence <SEQ ID 188> is:

| | |
|---|---|
| 1 | GTGGCGTCTG AAACGTATCC TTCTCAGATA TTGCACGCTC |
| | AGAGGGAAGT |
| 51 | ACGTGATGCC TATTTTAATC AAGCGGATTG CCATCCTGCT |
| | CGGGCTAATC |
| 101 | AGATTCTCGA GGCTAAGAAA ATCTGTTTAT TAGATGTTTA |
| | TCATACTAAT |
| 151 | CATTATTCCG TATTTACTTT TTGTGTAGAT AATTATCCGA |
| | ATCTCCGCTT |
| 201 | TACATTTGTA TCTTCAAAAA ACAATGAGAT GAATGGCTTA |
| | TCTAATCCTC |
| 251 | TAGATAATGT TCTTGTAGAG GCTATGGTAC GTAGAACACA |
| | TGCAAGAAAC |
| 301 | CTACTTGCAG CGTGTAAAAT TCGAAATATT GAGGTTCCAA |
| | GGGTTGTTGG |
| 351 | GCTTGACCTA AGATCTGGGA TACTCATTTC GAAACTAGAA |
| | TTGAAGCAAC |
| 401 | CTCAGTTCCA AAGTTTAACA GAAGACTTCG TAAATCATTC |
| | CACAAATCAG |
| 451 | GAAGAAGCTC GCGTCCATCA AAAGCATGTG TTGCTAATTT |
| | CTTTAATTTT |
| 501 | ACTTTGCAAG CAGGCCGTTC TGGAATCATT CCAGGAAAAA |
| | AAGCGATCCT |
| 551 | CTTAA |

The PSORT algorithm predicts inner membrane (0.2084).

The protein was expressed in E. coli and purified as a GST-fusion product (FIG. 94A) and also in his-tagged form. The recombinant proteins were used to immunise mice, whose sera were used in Western blots (FIG. 94B) and for FACS analyses (FIG. 94C; GST-fusion).

These experiments show that cp6449 is a surface-exposed and immunoaccessible protein, and that it is a useful immunogen. These properties are not evident from the sequence alone.

### Example 95

The following C. pneumoniae protein (PID 4376495) was expressed <SEQ ID 189; cp6495>:

MRELNAFELTQPEEYRNRWVLMPCLKCRFCRTQHAKVWSYRCVHEASLYEKNCELTLTYDDKHLPQYGSLVKLHLQLFLKR
LRKMISPHKIRYFECGAYGTKLQRPHYHLLLS

The cp6495 nucleotide sequence <SEQ ID 190> is:

TTGCGAGAATTAAATGCTTTTGAATTAACTCAACCTGAAGAGTATCGAAACCGTTGGGTTTTGATGCCTTGTCTTAAGTGT
CGTTTTTGTAGAACGCAACATGCAAAAGTCTGGTCTTATCGTTGTGTCCATGAAGCTTCTTTGTATGAGAAAAATTGTTTT
CTTACTTTGACTTATGATGATAAGCATTTACCTCAGTATGGTTCGTTGGTAAAGCTGCATTTACAGCTGTTTCTTAAGAGA
TTAAGAAAGATGATTTCTCCTCATAAAATTCGTTATTTTGAATGTGGTGCGTATGGAACCAAATTACAAAGACCTCATTAT
CATCTACTTTTATCATGA

The PSORT algorithm predicts cytoplasmic (0.280).

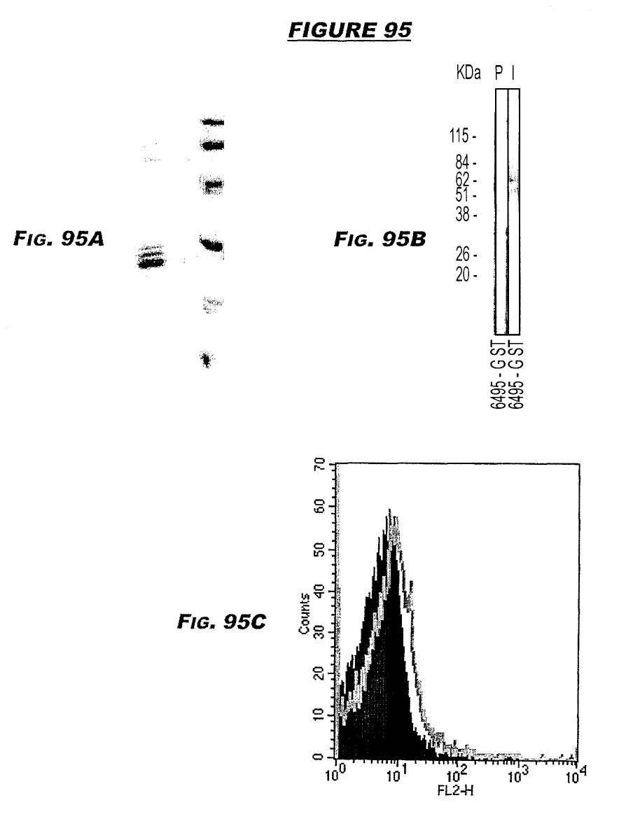

The protein was expressed in E. coli and purified as a GST-fusion product (FIG. 95A). The recombinant protein was used to immunise mice, whose sera were used in a Western blot (FIG. 95B) and for FACS analysis (FIG. 95C).

These experiments show that cp6495 is a surface-exposed and immunoaccessible protein, and that it is a useful immunogen. These properties are not evident from the sequence alone.

### Example 96

The following C. pneumoniae protein (PID 4376506) was expressed <SEQ ID 191; cp6506>:

| | |
|---|---|
| 1 | MRRFLFLILS SLRPVAFSAD NFTILEEKQS PLSRVSIIFA |
| | LPGVTPVSFD |
| 51 | GNCPIPWFSH SKKTLEGQRI YYSGDSFGKY FVVSALWPNK |
| | VSSAVVACNM |
| 101 | ILKHRVDLIL IIGSCYSRSQ DSRFGSVLVS KGYINYDADV |
| | RPFFERFEIP |
| 151 | DIKKSVFATS EVHREAILRG GEEFISTHKQ EIEELLKTHG |
| | YLKSTTKTEH |
| 201 | TLMEGLVATG ESFAMSRNYF LSLQKLYPEI HGFDSVSGAV |
| | SQVCYEYSIP |
| 251 | CLGVNILLPH PLESRENEDW KHLQSEASKI YMDTLLKSVL |
| | KELCSSH* |

The cp6506 nucleotide sequence <SEQ ID 192> is:

| | |
|---|---|
| 1 | ATGCGTCGTT TTCTGTTTCT TATTCTTAGC TCTCTTCCTT |
| | TGGTCGCATT |
| 51 | CTCTGCTGAT AGTTTCACTA TTCTAGAAGA AAAACAGAGT |
| | CCTTTAAGTC |
| 101 | GTGTAGGTAT TATTTTTGCT TTACCTGGGG TTACTCCCGT |
| | TTCTTTTGAT |
| 151 | GGTAATTGTC CTATTCCTTG GTTTTCTCAT AGTAAAAAGA |
| | CTCTAGAGGG |
| 201 | ACAGAGAATT TATTACTCTG GCGACTCCTT TGGGAAATAC |
| | TTTGTAGTTT |
| 251 | CTGCTCTTTG GCCTAATAAA GTTTCTTCAG CTGTTGTGGC |
| | TTGTAATATG |
| 301 | ATTCTTAAAC ATCGAGTGGA TCTTATTCTA ATTATAGGCT |
| | CGTGTTACTC |
| 351 | TAGGTCTCAA GATAGCCGTT TTGGCAGCGT CTTAGTTTCT |
| | AAAGGCTACA |
| 401 | TTAATTATGA TCCAGATGTG AGGCCTTTCT TTGAAAGATT |
| | TGAGATTCCA |
| 451 | GACATTAAAA AGAGTGTTTT TGCAACCAGT GAGGTTCATC |
| | GGGAGGCAAT |
| 501 | TCTTCGTGGA GGCGAAGAGT TTATTTCTAC CCATAAACAA |
| | GAAATCGAAG |
| 551 | AGCTTTTGAA GACTCATGGG TATTTGAAAT CAACAACCAA |
| | AACGGAGCAC |
| 601 | ACCTTAATGG AAGGTTTGGT TGCTACAGGC GAGTCTTTCG |
| | CGATGTCGCG |
| 651 | AAACTATTTT CTTTCCTTAC AAAAATTGTA TCCAGAGATT |
| | CATGGTTTTG |
| 701 | ATAGTGTCAG CGGCGCTGTT TCTCAGGTAT GCTATGAATA |
| | TAGCATTCCT |
| 751 | TGTTTAGGTG TGAATATCCT TCTCCCTCAT CCTTTAGAAT |
| | CACGGAGTAA |
| 801 | CGAGGATTGG AAGCATCTTC AAAGTGAGGC AAGTAAAATT |
| | TATATGGATA |
| 851 | CCTTGCTCAA GAGTGTATTA AAAGAACTCT GTTCTTCTCA |
| | TTAA |

The PSORT algorithm predicts periplasmic space (0.571).

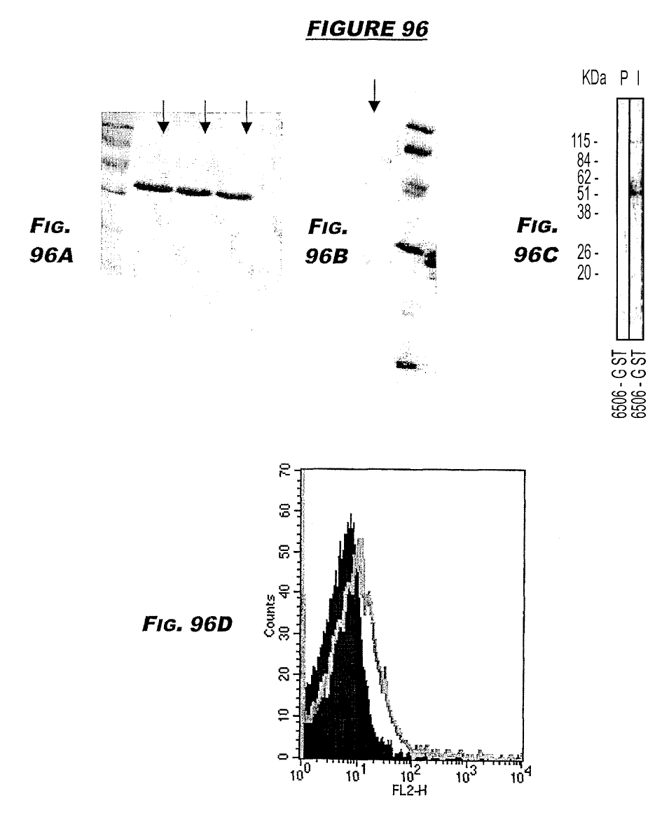

The protein was expressed in E. coli and purified as his-tag (FIG. 96A) and GST-fusion (FIG. 96B) products. The GST-fusion protein was used to immunise mice, whose sera were used in a Western blot (FIG. 96C) and for FACS analysis (FIG. 96D).

These experiments show that cp6506 is a surface-exposed and immunoaccessible protein, and that it is a useful immunogen. These properties are not evident from the sequence alone.

### Example 97

The following C. pneumoniae protein (PID 4376882) was expressed <SEQ ID 193; cp6882>:

| | |
|---|---|
| 1 | MSLLNLRSSQ DSASEDSTSQ SQIFDRIRNR ELVSTPEEKV |
| | RQRLLSFLMH |
| 51 | KLNYPKKLII IEKELKTLFP LLMRKGTLIP KRRPDILIIT |
| | PPTYTDAQGN |
| 101 | THNLGDPKPL LLIECKALAV NQNALKQLLS YNYSIGATCI |
| | AMAGKHSQVS |
| 151 | ALFNPKTQTL DFYPGLPEYS QLLNYFISLN L* |

The cp6882 nucleotide sequence <SEQ ID 194> is:

| | |
|---|---|
| 1 | ATGTCCTTAT TGAACCTTCC CTCAAGCCAG GATTCTGCAT |
| | CTGAGGACTC |
| 51 | CACATCGCAA TCTCAAATCT TCGATCCCAT TAGAAATCGG |
| | GAGTTAGTTT |
| 101 | CTACTCCCGA AGAAAAAGTC CGCCAAAGGT TGCTCTCCTT |
| | CCTAATGCAT |
| 151 | AAGCTGAACT ACCCTAAGAA ACTCATCATC ATAGAAAAAG |
| | AACTCAAAAC |
| 201 | TCTTTTTCCT CTGCTTATGC GTAAAGGAAC CCTAATCCCA |
| | AAACGCCGCC |
| 251 | CAGATATTCT CATCATCACT CCCCCCACAT ACACAGACGC |
| | ACAGGGAAAC |
| 301 | ACTCACAACC TAGGCGACCC AAAACCCCTG CTACTTATCG |
| | AATGTAAGGC |
| 351 | CTTAGCCGTA AACCAAAATG CACTCAAACA ACTCCTTAGC |
| | TATAACTACT |
| 401 | CTATCGGAGC CACCTGCATT GCTATGGCAG GGAAACACTC |
| | TCAAGTGTCA |
| 451 | GCTCTCTTCA ATCCAAAAAC ACAAACTCTT GATTTTTATC |
| | CTGGCCTCCC |
| 501 | AGAGTATTCC CAACTCCTAA ACTACTTTAT TTCTTTAAAC |
| | TTATAG |

The PSORT algorithm predicts cytoplasm (0.362).

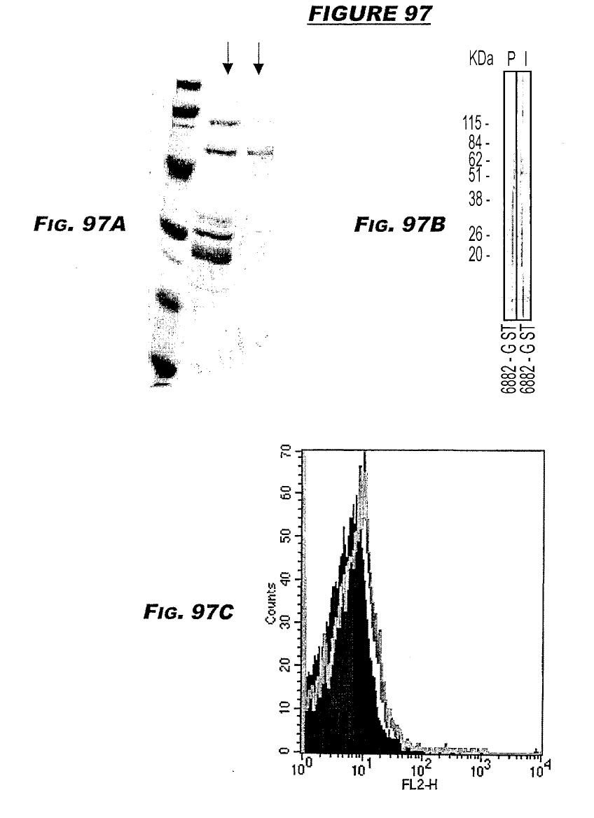

The protein was expressed in E. coli and purified as a GST-fusion product (FIG. 97A). The protein was used to immunise mice, whose sera were used in a Western blot (FIG. 97B) and for FACS analysis (FIG. 97C).

These experiments show that cp6882 is a surface-exposed and immunoaccessible protein, and that it is a useful immunogen. These properties are not evident from the sequence alone.

### Example 98

The following C. pneumoniae protein (PID 4376979) was expressed <SEQ ID 195; cp6979>:

| | |
|---|---|
| 1 | MSVNPSGNSK NDLWITGAHD QHPDVKESGV TSANLGSHRV |
| | TASGGRQGLL |
| 51 | ARIKEAVTGF FSRMSFFRSG APRGSQQPSA PSADTVRSPL |
| | PGGDARATEG |
| 101 | AGRNLIKKGY QPGMKVTIPQ VPGGGAQRSS GSTTLKPTRP |
| | APPPPKTGGT |
| 151 | NAKRPATHGK GPAPQPPKTG GTNAKRAATH GKGPAPQPPK |
| | GILKQPGQSG |
| 201 | TSGKKRVSWS DED* |

The cp6979 nucleotide sequence <SEQ ID 196> is:

| | |
|---|---|
| 1 | ATGTCTGTTA ATCCATCAGG AAATTCCAAG AACGATCTCT |
| | GGATTACGGG |
| 51 | AGCTCATGAT CAGCATCCCG ATGTTAAAGA ATCCGGGGTT |
| | ACAAGTGCTA |
| 101 | ACCTAGGAAG TCATAGAGTG ACTGCCTCAG GAGGACGCCA |
| | AGGGTTATTA |
| 151 | GCACGAATCA AAGAAGCAGT AACCGGGTTT TTTAGTCGGA |
| | TGAGCTTCTT |
| 201 | CAGATCGGGA GCTCCAAGAG GTAGCCAACA ACCCTCTGCT |
| | CCATCTGCAG |
| 251 | ATACTGTACG TAGCCCGTTG CCGGGAGGGG ATGCTCGCGC |
| | TACCGAGGGA |
| 301 | GCTGGTAGGA ACTTAATTAA AAAAGGGTAC CAACCAGGGA |
| | TGAAAGTCAC |
| 351 | TATCCCACAG GTTCCTGGAG GAGGGGCCCA ACGTTCATCA |
| | GGTAGCACGA |
| 401 | CACTAAAGCC TACGCGTCCG GCACCCCCAC CTCCTAAAAC |
| | GGGTGGAACT |
| 451 | AATGCAAAAC GTCCGGCAAC GCACGGGAAG GGTCCAGCAC |
| | CCCAGCCTCC |
| 501 | TAAAACAGGT GGGACCAATG CTAAGCGCGC AGCAACGCAT |
| | GGGAAAGGTC |
| 551 | CAGCACCTCA ACCTCCTAAG GGCATTTTGA AACAGCCTGG |
| | GCAGTCTGGG |
| 601 | ACTTCAGGAA AGAAGCGTGT CAGCTGGTCT GACGAAGATT |
| | AA |

The PSORT algorithm predicts cytoplasm (0.360).

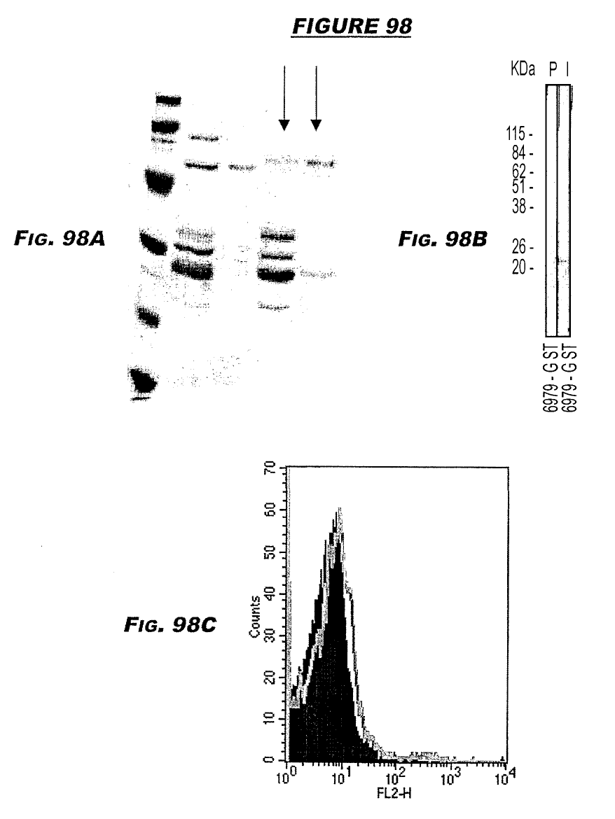

The protein was expressed in E. coli and purified as a GST-fusion product (FIG. 98A). The GST-fusion protein was used to immunise mice, whose sera were used in a Western blot (FIG. 98B) and for FACS analysis (FIG. 98C).

These experiments show that cp6979 is a surface-exposed and immunoaccessible protein, and that it is a useful immunogen. These properties are not evident from the sequence alone.

### Example 99

The following C. pneumoniae protein (PID 4377028) was expressed <SEQ ID 197; cp7028>:

| | |
|---|---|
| 1 | MLLGFLCDCP CASWQCAAVA NCYDSVFMSR PEHKPNIPYI |
| | TKATRRGLRM |
| 51 | KTLAYLASLK DARQLAYDFL KDPGSLARLA KALIAPKEAL |
| | QEGNLFFYGC |
| 101 | SNIEDILEEM RRPHRILLLG FSYCQKPKAC PEGRFNDACR |
| | YDPSHPTCAS |
| 151 | CSIGTMMRLN ARRYTTVIIP TFIDIAKHLH TLKKRYPGYQ |
| | ILFAVTACEL |
| 201 | SLKMFGDYAS VMNLKGVGIR LTGRICNTFK AFKLAERGVK |
| | PGVTILEEDG |
| 251 | FEVLARILTE YSSAPFPRDF CEIH* |

The cp7028 nucleotide sequence <SEQ ID 198> is:

| | |
|---|---|
| 1 | ATGCTTCTAG GGTTTTTGTG TGACTGCCCC TGTGCTTCGT |
| | GGCAGTGTGC |
| 51 | GGCCGTTGCT AATTGTTATG ATTCCGTATT TATGTCTAGA |
| | CCAGAGCACA |
| 101 | AACCTAATAT TCCTTATATT ACTAAAGCTA CAAGACGGGG |
| | TCTGCGTATG |
| 151 | AAGACGCTTG CTTATCTGGC CTCTTTAAAA GATGCTAGAC |
| | AGCTTGCCTA |
| 201 | TGATTTTCTG AAAGATCCTG GTTCTTTAGC TCGGTTAGCT |
| | AAGGCTTTGA |
| 251 | TAGCTCCTAA GGAGGCCTTA CAGGAGGGCA ACCTATTTTT |
| | TTATGGCTGT |
| 301 | AGTAATATTG AGGATATTTT AGAGGAGATG CGTCGTCCTC |
| | ATAGAATCCT |
| 351 | TTTGTTAGGA TTTTCTTATT GTCAAAAGCC TAAGGCATGT |
| | CCTGAAGGGC |
| 401 | GTTTCAATGA TGCTTGTCGG TATGATCCTT CACATCCTAC |
| | ATGTGCCTCA |
| 451 | TGTTCTATAG GGACCATGAT GCGGCTGAAT GCTCGTAGAT |
| | ACACTACTGT |
| 501 | GATCATCCCT ACATTTATAG ATAGCGCAAA ACATTTACAC |
| | ACTTTAAAAA |
| 551 | AGCGCTACCC TGGATATCAA ATTCTCTTTG CAGTTACTGC |
| | TTGTGAACTT |
| 601 | TCCTTAAAAA TGTTTGGAGA TTATGCCTCC GTAATGAACT |
| | TAAAGGGTGT |
| 651 | GGGCATCAGA CTCACAGGAC GTATTTGCAA TACATTTAAG |
| | GCATTTAAAT |
| 701 | TAGCTGAGCG AGGAGTCAAA CCAGGAGTCA CTATCCTAGA |
| | AGAAGATGGC |
| 751 | TTTGAGGTAT TAGCAAGGAT TCTTACAGAA TACAGTAGCG |
| | CTCCTTTCCC |
| 801 | TAGAGACTTT TGTGAGATCC ATTAG |

The PSORT algorithm predicts cytoplasm (0.1453).

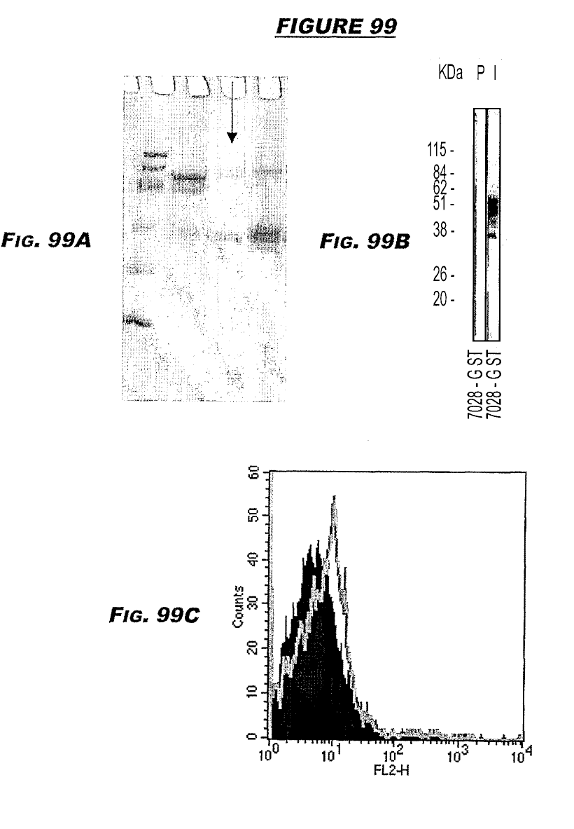

The protein was expressed in E. coli and purified as a GST-fusion product (FIG. 99A). The recombinant protein was used to immunise mice, whose sera were used in a Western blot (FIG. 99B) and for FACS analysis (FIG. 99C).

These experiments show that cp7028 is a surface-exposed and immunoaccessible protein, and that it is a useful immunogen. These properties are not evident from the sequence alone.

### Example 100

The following C. pneumoniae protein (PID 4377355) was expressed <SEQ ID 199; cp7355>:

| | |
|---|---|
| 1 | MKKVVTLSII FFATYCASEL SAVTVVAVPL SEAPGKIQVR |
| | PVVGLQFQEE |
| 51 | QGSVPYSFYY PYDYGYYYPE TYGYTKNTGQ ESRECYTRFE |
| | DGTIDYECD* |

The cp7355 nucleotide sequence <SEQ ID 200> is:

| | |
|---|---|
| 1 | ATGAAGAAAG TCGTAACACT ATCCATTATA TTTTTCGCAA |
| | CGTATTGTGC |
| 51 | ATCAGAGCTT AGTGCTGTAA CTGTAGTGGC TGTGCCTTTA |
| | TCAGAGGCTC |
| 101 | CAGGGAAGAT TCAAGTTCGT CCCGTCGTTG GTCTGCAATT |
| | TCAAGAAGAA |
| 151 | CAGGGTTCTG TGCCCTATAG TTTTTATTAT CCTTATGACT |
| | ATGGGTATTA |
| 201 | CTATCCAGAG ACTTATGGCT ATACTAAAAA TACAGGTCAA |
| | GAAAGTCGCG |
| 251 | AATGTTATAC CCGATTTGAA GATGGCACAA TTTTTTATGA |
| | ATGCGATTAG |

The PSORT algorithm predicts inner membrane (0.143).

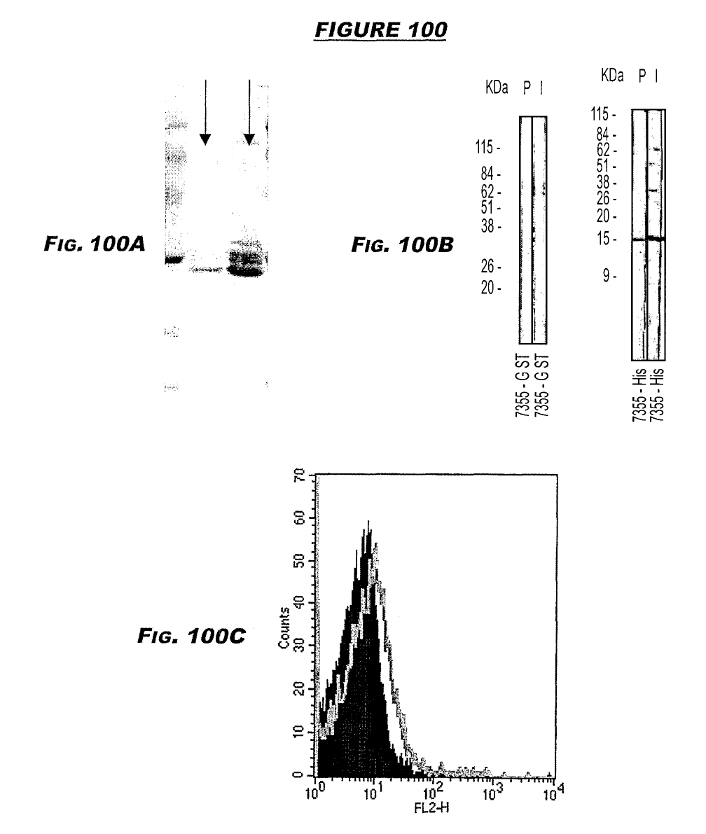

The protein was expressed in E. coli and purified as a GST-fusion (FIG. 100A) and a his-tag product. The proteins were used to immunise mice, whose sera were used in a Western blot (FIG. 100B) and for FACS analysis (FIG. 100C).

These experiments show that cp7355 is a surface-exposed and immunoaccessible protein, and that it is a useful immunogen. These properties are not evident from the sequence alone.

### Example 101

The following C. pneumoniae protein (PID 4377380) was expressed <SEQ ID 201; cp7380>:

| | |
|---|---|
| 1 | VHYCERTLDP KYILKIALKL RQSLSLFFQN SQSLQRAYST |
| | PYSYYRIILQ |
| 51 | KENKEKQALA RHKCISILEF FKNLLFVHLL SLSKNQREGC |
| | STDMAVVSTP |
| 101 | FFNRNLWYRL LSSRFSLWKS YCPRFFLDYL EAFGLLSDFL |
| | DHQAVIKFFE |
| 151 | LETHFSYYPV SGFVAPHQYL SLLQDRYFPI ASVMRTLDKD |
| | NFSLTPDLIH |
| 201 | DLLGHVPWLL HPSFSEFFIN MGRLFTKVIE KVQALPSKKQ |
| | RIQTLQSNLI |
| 251 | AIVRCFWFTV ESGLIENHEG RKAYGAVLIS SPQELGHAFI |
| | DNVRVLPLEL |
| 301 | DQIIRLRFNT STPQETLFSI RHFDELVELT SKLEWMLDQG |
| | LLESIPLYNQ |
| 351 | EKYLSGFEVL CQ* |

The cp7380 nucleotide sequence <SEQ ID 202> is:

| | |
|---|---|
| 1 | GTGCACTACT GCGAGAGAAC CCTGGACCCA AAGTATATTC |
| | TGAAGATTGC |
| 51 | TCTAAAGCTG AGACAATCAC TTTCCCTGTT CTTCCAGAAC |
| | AGCCAATCAC |
| 101 | TCCAACGTGC ATACTCGACC CCATATTCCT ACTACCGAAT |
| | CATTCTACAA |
| 151 | AAGGATAATA AAGAGAAGCA AGCTTTAGCT CGACACAAAT |
| | GCATTTCTAT |
| 201 | TTTAGAATTT TTCAAAAACT TACTCTTTGT TCATCTTCTG |
| | TCATTATCAA |
| 251 | AGAATCAAAG GGAAGGTTGC TCCACTGATA TGGCTGTTGT |
| | AAGCACTCCC |
| 301 | TTTTTTAATC GGAATTTATG GTATCGACTC CTTTCCTCAC |
| | GGTTTTCTCT |
| 351 | ATGGAAAAGC TATTGTCCAA GATTTTTTCT TGATTACTTA |
| | GAAGCTTTCG |
| 401 | GTCTCCTTTC TGATTTCTTA GACCATCAAG CAGTCATTAA |
| | ATTCTTCCAA |
| 451 | TTAGAAACAC ATTTTTCCTA TTATCCCGTT TCAGGATTTG |
| | TAGCTCCCCA |
| 501 | TCAATACTTG TCTCTGTTGC AGGACCGTTA CTTTCCCATT |
| | GCCTCTGTAA |
| 551 | TGCGAACTCT CGATAAAGAT AGTTTCTCCT TAACTCCTGA |
| | TCTCATCCAT |
| 601 | GACCTTTTAG GGCACGTGCC TTGGCTTCTA CATCCCTCAT |
| | TTTCTGAATT |
| 651 | TTTCATAAAC ATGGGAAGAC TCTTCACTAA AGTCATAGAA |
| | AAAGTACAAG |
| 701 | CTCTTCCTAG TAAAAAACAA CGCATACAAA CCCTACAAAG |
| | CAATCTGATC |
| 751 | GCTATTGTAC GCTGCTTTTG GTTTACTGTT GAAAGCGGAC |
| | TTATTGAAAA |
| 801 | CCATGAAGGA AGAAAAGCAT ATGGAGCCGT TCTTATCAGT |
| | TCTCCTCAGG |
| 851 | AACTTGGACA CGCTTTCATT GATAACGTAC GTGTTCTCCC |
| | TTTAGAATTG |
| 901 | GATCAGATTA TTCGTCTTCC CTTCAATACA TCAACTCCAC |
| | AAGAGACTTT |
| 951 | ATTTTCAATA AGACATTTTG ATGAACTGGT AGAACTCACT |
| | TCAAAATTAG |
| 1001 | AATGGATGCT CGACCAAGGT CTGTTAGAAT CAATTCCCCT |
| | TTACAATCAA |
| 1051 | GAGAAATATC TTTCTGGTTT TGAGGTACTT TGCCAATGA |

The PSORT algorithm predicts inner membrane (0.1362).

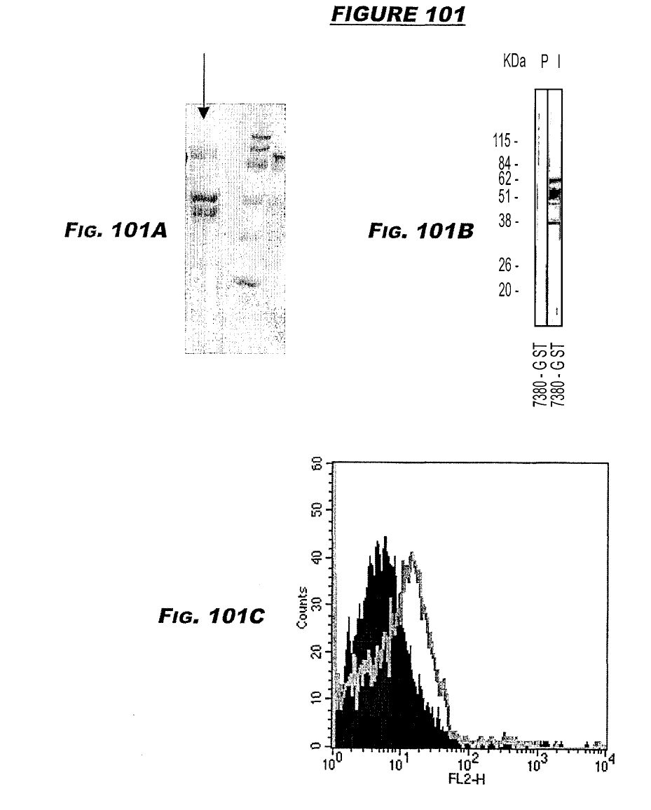

The protein was expressed in E. coli and purified as a GST-fusion product (FIG. 101A). The recombinant protein was used to immunise mice, whose sera were used in a Western blot (FIG. 101B) and for FACS analysis (FIG. 101C).

These experiments show that cp7380 is a surface-exposed and immunoaccessible protein, and that it is a useful immunogen. These properties are not evident from the sequence alone.

### Example 102

The following C. pneumoniae protein (PID 4376904) was expressed <SEQ ID 203; cp6904>:

| | |
|---|---|
| 1 | MMNYEDAKLR GQAVAILYQI GAIKFGKHIL ASGEETPLYV |
| | DMRLVISSPE |
| 51 | VLQTVATLIW RLRPSFNSSL LCGVRYTALT LATSISLKYN |
| | IPMVLRRKEL |
| 101 | QNVDRSDAIK VEGLFTPGQT CLVINDMVSS GKSIIETAVA |
| | LEENGLVVRE |
| 151 | ALVFLDRRKE ACQPLGPQGI KVSSVFTVPT LIKALIAYGK |
| | LSSGDLTLAN |
| 201 | KISEILEIES * |

The cp6904 nucleotide sequence <SEQ ID 204> is:

| | |
|---|---|
| 1 | ATGATGAACT ACGAAGATGC AAAATTACGC GGTCAAGCTG |
| | TAGCAATTCT |
| 51 | ATACCAAATC GGAGCTATAA AGTTCGGAAA ACATATTCTC |
| | GCTAGCGGAG |
| 101 | AAGAAACTCC TCTGTATGTA GATATGCGTC TTGTGATCTC |
| | CTCTCCAGAA |
| 151 | GTTCTCCAGA CAGTGGCAAC TCTTATTTGG CGCCTCCGCC |
| | CCTCATTCAA |
| 201 | TAGTAGCTTA CTCTGCGGAG TCCCTTATAC TGCTCTAACC |
| | CTAGCAACCT |
| 251 | CGATCTCTTT AAAATATAAC ATCCCTATGG TATTGCGAAG |
| | GAAGGAATTA |
| 301 | CAGAATGTAG ACCCCTCGGA CGCTATTAAA GTAGAAGGGT |
| | TATTTACTCC |
| 351 | AGGACAAACT TGTTTAGTCA TCAATGATAT GGTTTCCTCA |
| | GGAAAATCTA |
| 401 | TAATAGAGAC AGCAGTCGCA CTGGAAGAAA ATGGTCTGGT |
| | AGTTCGTGAA |
| 451 | GCATTGGTAT TCTTAGATCG TAGAAAAGAA GCGTGTCAAC |
| | CACTTGGTCC |
| 501 | ACAGGGAATA AAAGTCAGTT CGGTATTTAC TGTACCCACT |
| | CTGATAAAAG |
| 551 | CTTTGATCGC TTATGGGAAG CTAAGCAGTG GTGATCTAAC |
| | CCTGGCAAAC |
| 601 | AAAATTTCCG AAATTCTAGA AATTGAATCT TAA |

The PSORT algorithm predicts cytoplasm (0.0358).

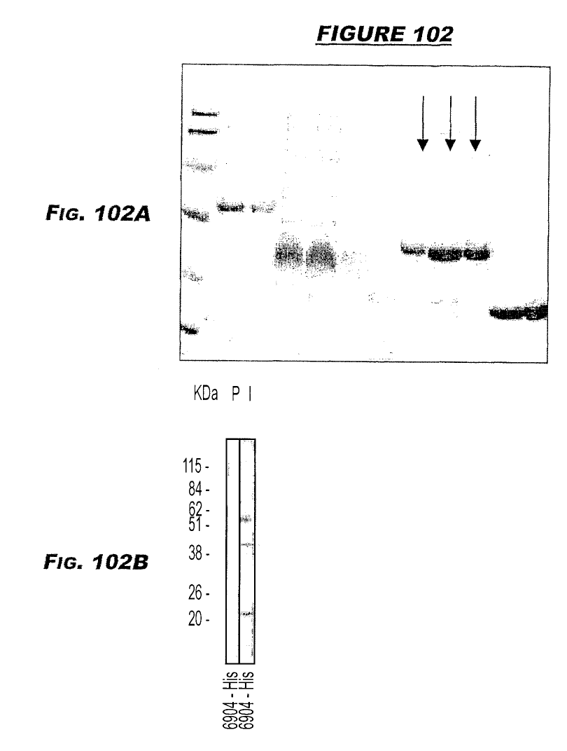

The protein was expressed in E. coli and purified as a his-tag product (FIG. 102A). The recombinant protein was used to immunise mice, whose sera were used in a Western blot (FIG. 102B) and for FACS analysis.

The cp6904 protein was also identified in the 2D-PAGE experiment.

These experiments show that cp6904 is a surface-exposed and immunoaccessible protein, and that it is a useful immunogen. These properties are not evident from the sequence alone.

### Example 103

The following C. pneumoniae protein (PID 4376964) was expressed <SEQ ID 205; cp6964>:

| | |
|---|---|
| 1 | MKKLIALIGI FLVPIKGNTN KEHDAHATVL KAARAKYNLF |
| | FVQVFPVHE |
| 51 | VIEPISPDCL VHYEGWV* |

The cp6964 nucleotide sequence <SEQ ID 206> is:

| | |
|---|---|
| 1 | ATGAAAAAAT TGATTGCTTT GATAGGGATA TTTCTTGTTC |
| | CAATAAAAGG |
| 51 | AAATACCAAT AAGGAACACG ACGCTCACGC GACTGTTTTA |
| | AAAGCGGCCA |
| 101 | GAGCAAAGTA TAATTTGTTC TTTGTTCAGG ATGTTTTCCC |
| | TGTACACGAA |
| 151 | GTTATCGAGC CTATTTCTCC CGATTGCCTG GTACATTATG |
| | AAGGGTGGGT |
| 201 | TTGA |

The PSORT algorithm predicts inner membrane (0.091).

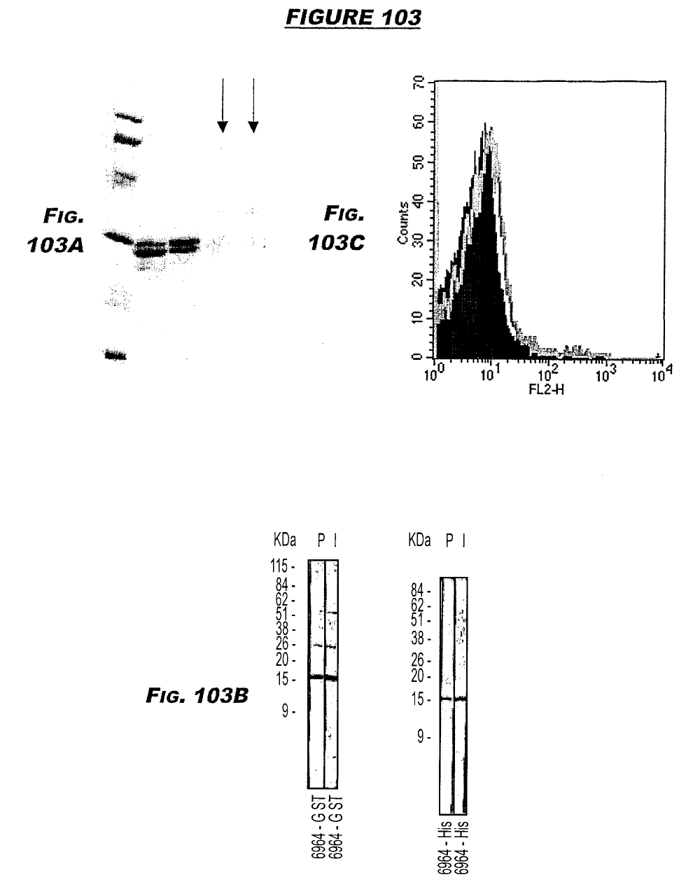

The protein was expressed in E. coli and purified as a GST-fusion product (FIG. 103A) and also in his-tagged form. The recombinant proteins were used to immunise mice, whose sera were used in a Western blot (FIG. 103B) and for FACS analysis (FIG. 103C).

These experiments show that cp6964 is a surface-exposed and immunoaccessible protein, and that it is a useful immunogen. These properties are not evident from the sequence alone.

### Example 104

The following C. pneumoniae protein (PID 4377387) was expressed <SEQ ID 207; cp7387>:

| | |
|---|---|
| 1 | LNFAKIDHNH LYLTCLGDLG VACPILSTDC LPNYSEKASH |
| | EVLVYSKFRC |
| 51 | ISGFPSRLAT SGNDTYYSIV SLPIGLRYEV TSPSGRHDFN |
| | IDMHVAPKIG |
| 101 | AVLSHGTREA KEIPGSSKDY AFFSLTARES LMISEKLAMT |
| | FQVSEVIQNC |
| 151 | YSQCTKVTKT NLKEQYRHLS HNTGFELSVK SAF* |

The cp7387 nucleotide sequence <SEQ ID 208> is:

| | |
|---|---|
| 1 | TTGAATTTTG CAAAGATTGA TCACAATCAT CTCTACCTTA |
| | CATGTTTGGG |
| 51 | AGATCTTGGT GTAGCTTGTC CTATACTTTC TACAGATTGT |
| | CTACCTAATT |
| 101 | ATAGCGAGAA AGCATCTCAT GAGGTTCTTG TTTATAGTAA |
| | ATTTAGATGC |
| 151 | ATTTCTGGAG AGCCATCTCG ACTTGCAACT TCAGGAAATG |
| | ACACATATTA |
| 201 | TTCTATAGTA AGTTTACCTA TAGGACTCCG TTACGAAGTG |
| | ACTTCACCAT |
| 251 | CAGGACGTCA TGATTTCAAT ATTGATATGC ATGTAGCTCC |
| | AAAGATAGGT |
| 301 | GCAGTACTCT CTCATGGAAC ACGAGAGGCT AAAGAGATCC |
| | CAGGATCTTC |
| 351 | AAAAGACTAT GCATTTTTTA GCTTGACTGC TAGAGAAAGT |
| | TTAATGATTT |
| 401 | CTGAAAAGCT TGCGATGACT TTCCAAGTTA GCGAAGTTAT |
| | TCAGAATTGT |
| 451 | TATTCACAAT GTACTAAAGT AACGAAAACT AATTTAAAAG |
| | AACAGTATAG |
| 501 | GCACTTATCC CACAATACAG GGTTTGAGTT AAGCGTCAAG |
| | TCTGCATTCT |
| 551 | AA |

The PSORT algorithm predicts inner membrane (0.043).

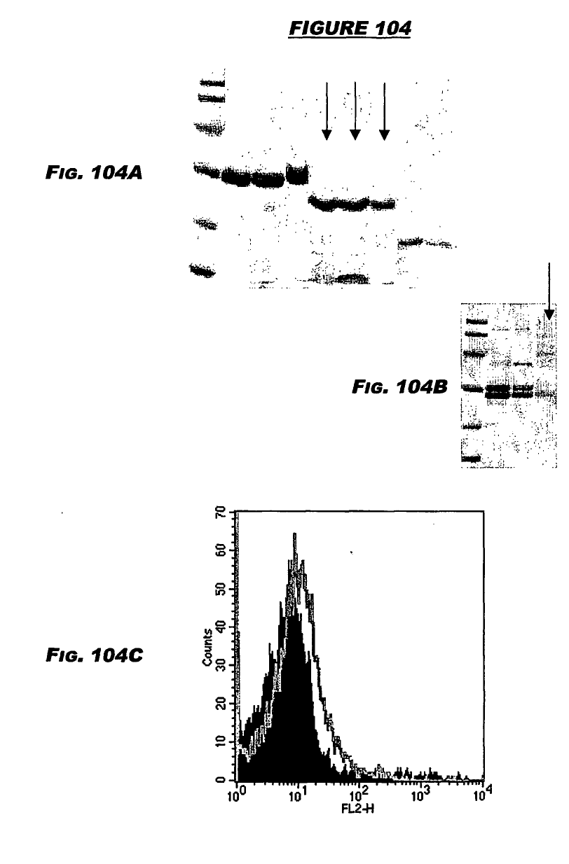

The protein was expressed in E. coli and purified as a his-tagged-fusion product (FIG. 104A) and also as a GST-fusion (FIG. 104B). The recombinant proteins were used to immunise mice, whose sera were used in a Western blot and for FACS analysis (FIG. 104C; his-tagged).

These experiments show that cp7387 is a surface-exposed and immunoaccessible protein, and that it is a useful immunogen. These properties are not evident from the sequence alone.

### Example 105

The following C. pneumoniae protein (PID 4376281) was expressed <SEQ ID 209; cp6281>:

| | |
|---|---|
| 1 | MFLQFFHPIV FSDQSLSFLP YLGKSSGIIE KCSNIVEHYL |
| | HLGGDTSVII |
| 51 | TGVSGATFLS VDHALPISKS EKIIKILSYI LILPLILALF |
| | IKIVLRIILF |
| 101 | FKYRGLILDV KKEDLKKTLT PDQENLSLPL PSPTTLKKIH |
| | ALHILVRSGK |
| 151 | TYNELIQEGF SFTKITDLGQ APSPKQDIGF SYNSLLPNFY |
| | FHSLVSVPNI |
| 201 | SGEERALNYH KEQQEEMAVK LKTMQACSFV FRSLHLPSMQ |
| | TKDKKAGFGL |
| 251 | LTFPPWKIYP I* |

The cp6281 nucleotide sequence <SEQ ID 210> is:

| | |
|---|---|
| 1 | ATGTTTCTTC AGTTTTTTCA TCCTATAGTC TTCTCGGATC |
| | AGTCCTTATC |
| 51 | TTTTCTTCCT TACCTAGGAA AAAGCTCTGG CATTATTGAA |
| | AAATGTTCCA |
| 101 | ATATCGTTGA ACACTATTTA CATTTGGGAG GAGACACTTC |
| | TGTTATCATC |
| 151 | ACAGGAGTTT CTGGAGCTAC CTTTCTATCT GTTGATCATG |
| | CCCTCCCAAT |
| 201 | CTCGAAATCT GAAAAAATAA TAAAAATTCT GCTTCATATT |
| | TTAATTCTTC |
| 251 | CTCTGATTCT AGCTCTCTTT ATTAAGATCG TTTTACGCAT |
| | TATCTTATTC |
| 301 | TTCAAGTATC GTGGTCTAAT CCTAGATGTT AAGAAGGAGG |
| | ATTTGAAAAA |
| 351 | AACACTTACA CCTGACCAAG AAAACCTCAG TCTTCCTTTA |
| | CCATCTCCTA |
| 401 | CAACATTAAA GAAAATCCAT GCGCTACACA TTTTAGTGCG |
| | TTCTGGAAAA |
| 451 | ACCTATAACG AGCTTATACA AGAAGGGTTT TCTTTCACTA |
| | AAATCACAGA |
| 501 | TCTTGGTCAA GCTCCTTCAC CAAAGCAAGA TATTGGCTTC |
| | TCTTATAATT |
| 551 | CCCTTCTCCC TAACTTCTAT TTTCATTCCT TGGTATCTGT |
| | TCCAAATATT |
| 601 | TCAGGCGAGG AACGGGCTCT TAATTATCAT AAAGAACAAC |
| | AAGAGGAAAT |
| 651 | GGCTGTTAAA TTAAAAACAA TGCAAGCGTG TTCTTTTGTC |
| | TTCCGATCCC |
| 701 | TGCATTTACC TTCAATGCAA ACGAAGGACA AAAAGGCTGG |
| | ATTTGGACTA |
| 751 | CTGACGTTTT TCCCTTGGAA AATCTACCCC CTATAA |

The PSORT algorithm predicts inner membrane (0.5373).

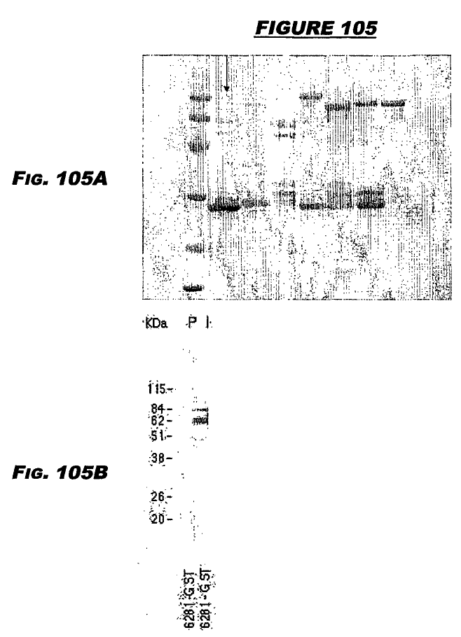

The protein was expressed in E. coli and purified as a GST-fusion product (FIG. 105A). The recombinant protein was used to immunise mice, whose sera were used in a Western blot (FIG. 105B) and for FACS analysis.

These experiments show that cp6281 is a surface-exposed and immunoaccessible protein, and that it is a useful immunogen. These properties are not evident from the sequence alone.

### Example 106 and Example 107

The following C. pneumoniae protein (PID 4376306) was expressed <SEQ ID 211; cp6306>:

| | |
|---|---|
| 1 | MGNHETYIHP GVLPSSHAQD VSRSTVYPSR SFIMRRMLMG |
| | WNFNRVPSKS |
| 51 | SEQLMDGHRI PLIFFGKHHP TISILNVNRF SWLSIFYNGE |
| | RGF* |

The cp6306 nucleotide sequence <SEQ ID 212> is:

| | |
|---|---|
| 1 | ATGGGAAACC ATGAGACCTA TATACATCCA GGAGTGCTCC |
| | CGAGTAGTCA |
| 51 | TGCTCAGGAT GTTAGCAGAT CTACAGTTTA CCCCAGTCGA |
| | AGTTTTATCA |
| 101 | TGAGACGTAT GCTCATGGGC TGGAATTTCA ATCGTGTTCC |
| | CTCGAAGAGC |
| 151 | TCCGAGCAGT TAATGGATGG TCATCGCATA CCTCTTATAT |
| | TTTTTGGGAA |
| 201 | GCATCATCCT ACTATATCTA TTTTAAATGT CAATAGATTT |
| | TCTTGGCTCT |
| 251 | CCATTTTTTA CAATGGAGAA AGGGGGTTTT GA |

The PSORT algorithm predicts cytoplasm (0.167).

The following C. pneumoniae protein (PID 4376434) was also expressed <SEQ ID 213; cp6434>:

| | |
|---|---|
| 1 | MSESINRSIH LEASTPFFIK LTNLCESRLV LITSLVISLL |
| | ALVGAGVTLV |
| 51 | VLFVAGILPL LPVLILEIIL ITVLVLLFCL VLEPYLIEKP |
| | SKIKELPKVD |
| 101 | ELSVVETDST L* |

The cp6434 nucleotide sequence <SEQ ID 214> is:

| | |
|---|---|
| 1 | ATGTCTGAAA GTATTAACAG AAGCATTCAT TTAGAAGCCT |
| | CTACACCATT |
| 51 | TTTTATAAAA TTAACGAATC TCTGTGAAAG TAGATTAGTT |
| | AAGATCACTT |
| 101 | CTCTTGTTAT TTCTCTATTA GCTTTAGTGG GTGCGGGAGT |
| | CACTCTTGTG |
| 151 | GTTTTATTTG TAGCTGGGAT CCTTCCTTTA CTTCCTGTAC |
| | TCATCTTAGA |
| 201 | AATTATTTTA ATAACCGTCC TTGTCTTGCT TTTTTGTTTG |
| | GTATTGGAAC |
| 251 | CTTATTTAAT AGAAAAACCT AGTAAAATAA AGGAACTACC |
| | TAAAGTAGAC |
| 301 | GAGCTATCTG TAGTAGAAAC GGACAGTACT CTTTAA |

The PSORT algorithm predicts inner membrane (0.6859).

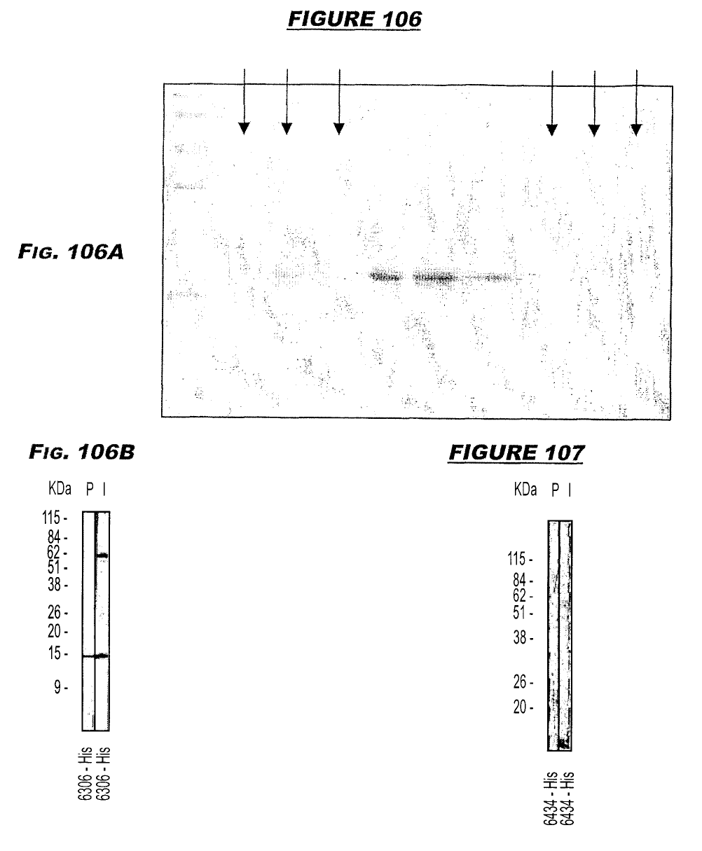

The proteins were expressed in E. coli and purified as his-tag products (FIG. 106A; 6306=lanes 2-4; 6434=lanes 8-10). The recombinant proteins were used to immunise mice, whose sera were used in Western blots (FIGS. 106B & 107) and for FACS analysis.

These experiments show that cp6306 & cp6434 are surface-exposed and immunoaccessible proteins, and that they are useful immunogens. These properties are not evident from the sequences alone.

### Example 108

The following C. pneumoniae protein (PID 4377400) was expressed <SEQ ID 215; cp7400>:

| | |
|---|---|
| 1 | MRVMRFFCLF ELGFLGSFHC VAEDKGVDLF GVWDDNQITE |
| | CDDSYMTEGR |
| 51 | EEVEKVVDA |

The cp7400 nucleotide sequence <SEQ ID 216> is:

| | |
|---|---|
| 1 | GTGAGAGTTA TGAGATTTTT TTGTCTATTT TTTCTTGGGT |
| | TCCTAGGATC |
| 51 | TTTTCATTGT GTTGCTGAAG ACAAGGGCGT GGATTTATTT |
| | GGAGTCTGGG |
| 101 | ACGATAACCA AATTACAGAG TGTGACGATA GTTACATGAC |
| | AGAGGGTCGT |
| 151 | GAAGAGGTTG AAAAGGTAGT GGACGCTTAG |

The PSORT algorithm predicts periplasmic space (0.924).

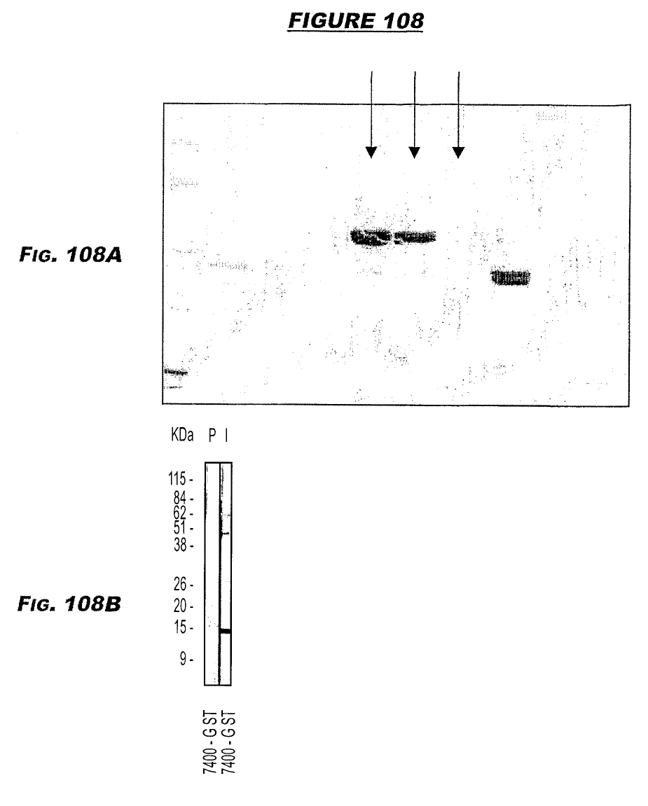

The protein was expressed in E. coli and purified as a GST-fusion product (FIG. 108A). The recombinant protein was used to immunise mice, whose sera were used in a Western blot (FIG. 108B) and for FACS analysis.

These experiments show that cp7400 is a surface-exposed and immunoaccessible protein, and that it is a useful immunogen. These properties are not evident from the sequence alone.

### Example 109

The following C. pneumoniae protein (PID 4376395) was expressed <SEQ ID 217; cp6395>:

| | |
|---|---|
| 1 | MENAMSSSFV YNGPSWILKT SVAQEVFKKH GKGIQVLLST |
| | SVMLFIGLGV |
| 51 | CAFIFPQYLI VFVLTIALLM LAISLVLFLL IRSVRSSMVD |
| | RLWCSEKGYA |
| 101 | LHQHENGPFL DVKRVQQILL RSPYIKVRAL WPSGDIPEDP |
| | SQAAVLLLSP |
| 151 | WTFFSSVDVE ALLPSPQEKE GKYIDPVLPK LSRIERVSLL |
| | VELSAFTLDD |
| 201 | LNEQGVNPLM NNEEFLFFIN KKAREHGIQD LKHEIMSSLE |
| | KTGVPLDPSM |
| 251 | SFQVSQAMFS VYRYLRQRDL TTSELRCFHL LSCFKGDVVH |
| | CLASFENPKD |
| 301 | LADSDFLEAC KNVEWGEFIS ACEKALLKNP QGISIKDLKQ |
| | FLVR* |

The cp6395 nucleotide sequence <SEQ ID 218> is:

| | |
|---|---|
| 1 | ATGGAGAATG CTATGTCATC ATCGTTTGTG TATAATGGGC |
| | CTTCGTGGAT |
| 51 | TTTAAAAACG TCAGTAGCTC AGGAGGTATT TAAAAAGCAC |
| | GGTAAGGGGA |
| 101 | TTCAGGTTCT CTTAAGTACT TCAGTGATGC TTTTTATAGG |
| | TCTTGGAGTC |
| 151 | TGTGCCTTTA TATTTCCTCA ATATCTGATT GTTTTTGTTT |
| | TGACTATAGC |
| 201 | TTTGCTTATG CTCGCTATAA GCTTGGTATT GTTTCTCTTA |
| | ATACGTTCTG |
| 251 | TACGCTCTTC AATGGTAGAT CGTTTGTGGT GTTCTGAAAA |
| | AGGATATGCT |
| 301 | CTTCATCAAC ATGAGAACGG GCCTTTTTTG GATGTGAAGC |
| | GTGTACAGCA |
| 351 | AATTCTTCTA AGATCACCCT ATATTAAAGT TCGGGCTTTA |
| | TGGCCGTCTG |
| 401 | GAGATATCCC TGAGGATCCT TCACAAGCTG CGGTTCTATT |
| | ACTTTCTCCT |
| 451 | TGGACTTTCT TTTCATCCGT GGATGTAGAG GCTTTATTAC |
| | CGAGTCCTCA |
| 501 | AGAAAAGGAG GGTAAGTATA TAGATCCTGT GCTGCCTAAG |
| | TTGTCTAGGA |
| 551 | TAGAGAGAGT CTCACTTTTA GTGTTTTTGA GTGCATTTAC |
| | TTTGGATGAC |
| 601 | TTAAACGAAC AGGGAGTCAA TCCTTTGATG AATAATGAGG |
| | AATTTTTATT |
| 651 | TTTTATAAAT AAGAAAGCGC GTGAGCATGG GATTCAGGAT |
| | TTAAAACACG |
| 701 | AGATTATGTC TTCGTTAGAG AAAACAGGAG TGCCATTAGA |
| | CCCCTCAATG |
| 751 | AGTTTTCAAG TTTCACAAGC GATGTTTTCT GTATATCGCT |
| | ACTTGAGACA |
| 801 | AAGGGATTTA ACGACTTCAG AATTAAGATG TTTTCACCTC |
| | TTAAGTTGTT |
| 851 | TTAAAGGGGA TGTGGTTCAT TGTTTAGCTT CATTTGAAAA |
| | CCCTAAAGAT |
| 901 | TTAGCAGATT CTGACTTTTT AGAAGCTTGT AAGAACGTGG |
| | AATGGGGTGA |
| 951 | GTTTATTTCG GCATGTGAGA AGGCTCTTTT AAAGAATCCG |
| | CAAGGAATTT |
| 1001 | CCATTAAGGA TCTAAAACAA TTTTTAGTGA GGTAA |

The PSORT algorithm predicts inner membrane (0.6307).

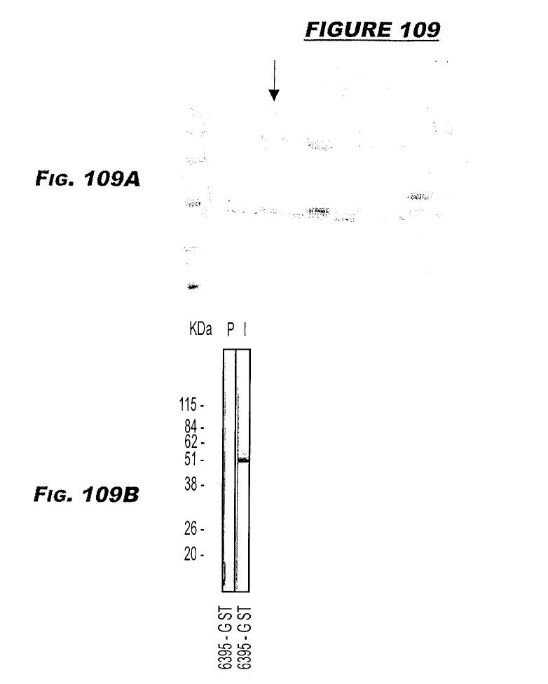

The protein was expressed in E. coli and purified as a GST-fusion product (FIG. 109A). The recombinant protein was used to immunise mice, whose sera were used in a Western blot (FIG. 109B) and for FACS analysis.

These experiments show that cp6395 is a surface-exposed and immunoaccessible protein, and that it is a useful immunogen. These properties are not evident from the sequence alone.

### Example 110

The following C. pneumoniae protein (PID 4376396) was expressed <SEQ ID 219; cp6396>:

| | |
|---|---|
| 1 | MIEFAFVPHT SVTADRIEDR MACRMNKLST LAITSLCVLI |
| | SSVCIMIGIL |
| 51 | CISGTVGTYA FVVGIIFSVL ALVACVFFLY FFYFSSEEFK |
| | CASSQEFRFL |
| 101 | PIPAVVSALR SYEYISQDAI NDVIKDTMQL STLSSLLDPE |
| | AFFLEFPYFN |
| 151 | SLIVNHSMKE ADRLSREAFL ILLGEITWKD CEIKILPWLK |
| | DPNITPDDFW |
| 201 | KLLKDHFDLK DFKKRIATWI RKAYPEIRLP KKHCLDKSIY |
| | KGCCKFLLLS |
| 251 | ENDVQYQRLL HKVCYFSGEF PAMVLGLGSE VPMVLGLPKV |
| | PKDLTWEMFM |
| 301 | ENMPVLLQSK REGHWKISLE DVASL* |

The cp6396 nucleotide sequence <SEQ ID 220> is:

| | |
|---|---|
| 1 | ATGATCGAGT TTGCTTTTGT TCCTCATACC TCCGTGACAG |
| | CGGATCGGAT |
| 51 | TGAGGATCGC ATGGCCTGTC GCATGAACAA GTTGTCTACT |
| | TTAGCAATTA |
| 101 | CAAGTCTTTG TGTATTGATC AGTTCAGTTT GTATTATGAT |
| | TGGGATTTTA |
| 151 | TGCATTTCTG GAACGGTTGG GACCTATGCA TTTGTTGTAG |
| | GAATTATTTT |
| 201 | TTCTGTCCTT GCTTTGGTAG CATGTGTTTT CTTTCTTTAT |
| | TTCTTTTATT |
| 251 | TTTCTTCTGA GGAATTTAAG TGTGCTTCTT CGCAGGAGTT |
| | TCGTTTTTTG |
| 301 | CCTATACCAG CTGTGGTTTC TGCATTGCGT TCCTATGAAT |
| | ACATTTCTCA |
| 351 | GGACGCTATC AATGACGTTA TAAAAGATAC GATGCAGTTG |
| | TCTACCCTTT |
| 401 | CTTCTCTTTT AGATCCCGAA GCTTTTTTCT TAGAATTTCC |
| | TTATTTTAAC |
| 451 | TCTTTGATAG TGAATCATTC GATGAAGGAA GCGGATCGTT |
| | TGTCTCGAGA |
| 501 | GGCTTTTTTG ATTTTATTAG GTGAGATTAC TTGGAAGGAT |
| | TGTGAAACAA |
| 551 | AAATTTTGCC ATGGTTGAAA GATCCTAATA TCACTCCTGA |
| | TGATTTCTGG |
| 601 | AAGCTATTAA AAGACCATTT CGATTTAAAG GACTTTAAGA |
| | AGAGGATCGC |
| 651 | CACTTGGATA CGGAAGGCCT ATCCAGAAAT TAGATTACCG |
| | AAGAAGCATT |
| 701 | GTTTAGATAA GTCTATCTAT AAGGGGTGTT GTAAGTTTTT |
| | ATTACTTTCT |
| 751 | GAGAATGATG TGCAATATCA GAGGTTATTA CATAAGGTCT |
| | GTTATTTCTC |
| 801 | TGGGGAGTTT CCTGCCATGG TTTTAGGTTT GGGAAGTGAA |
| | GTGCCTATGG |
| 851 | TGTTAGGACT CCCTAAGGTT CCCAAGGATC TTACCTGGGA |
| | GATGTTTATG |
| 901 | GAAAATATGC CTGTTCTTCT GCAAAGCAAA AGAGAGGGGC |
| | ATTGGAAAAT |
| 951 | CTCCTTGGAA GACGTAGCCT CTCTTTAA |

The PSORT algorithm predicts inner membrane (0.6095).

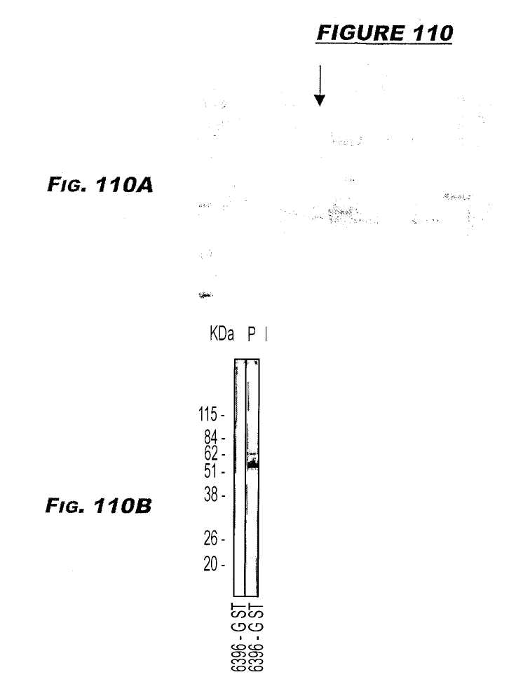

The protein was expressed in E. coli and purified as a GST-fusion product (FIG. 110A). The recombinant protein was used to immunise mice, whose sera were used in a Western blot (FIG. 110B) and for FACS analysis.

These experiments show that cp6396 is a surface-exposed and immunoaccessible protein, and that it is a useful immunogen. These properties are not evident from the sequence alone.

### Example 111

The following C. pneumoniae protein (PID 4376408) was expressed <SEQ ID 221; cp6408>:

| | |
|---|---|
| 1 | MNTSLKRPLK SHFDVVGSFL RPEHLKKTRE SLKEGSISLD |
| | QLMQIEDIAI |
| 51 | QDLIKKQKAA GLSFITDGEF RRATWHYDFM WGFEGVGHHR |
| | ATEGVFFDGE |
| 101 | RAMIDDTYLT DKISVSHHPF VDHFKFVKAL EDEFTTAKQT |
| | LPAPAQFLKQ |
| 151 | MIFPNNIEVT RKFYPTNQEL IEDIVAGYRK VIRDLYDAGC |
| | RYLQLDDCTR |
| 201 | GGLVDPRVCS WYGIDEKGLQ DLIQQYLLIN NLVIADRPDD |
| | LVVNLHVCRG |
| 251 | NYHSKFFASG SYDFIAKPLF EQTNVDGYYL EFDEERSGDF |
| | SPLTFISGEK |
| 301 | TVCLGLVTSK TPTLENKDEV IARIHQAADY LPLERLSLSP |
| | QCGFASCEIG |
| 351 | NKLTEEEQWA KVALVKEISE EVWK* |

The cp6408 nucleotide sequence <SEQ ID 222> is:

| | |
|---|---|
| 1 | ATGAATACTT CACTAAAAAG ACCTCTGAAA TCTCATTTTG |
| | ATGTTGTCGG |
| 51 | TAGTTTTTTG CGTCCTGAGC ATTTAAAAAA AACTAGAGAA |
| | AGCCTTAAAG |
| 101 | AAGGCTCTAT TTCTCTAGAT CAACTCATGC AAATTGAGGA |
| | TATCGCTATC |
| 151 | CAAGATTTGA TCAAAAAACA AAAAGCAGCA GGTCTTTCTT |
| | TTATTACTGA |
| 201 | TGGAGAATTC CGCAGAGCTA CGTGGCATTA CGACTTCATG |
| | TGGGGTTTTC |
| 251 | ATGGCGTAGG TCACCACAGA GCTACAGAAG GAGTTTTCTT |
| | TGATGGAGAA |
| 301 | CGCGCTATGA TCGATGATAC CTATCTGACA GACAAGATCT |
| | CTGTATCTCA |
| 351 | CCACCCATTT GTGGATCACT TTAAATTTGT AAAAGCTCTA |
| | GAAGATGAAT |
| 401 | TTACGACTGC AAAGCAAACT CTTCCTGCAC CGGCACAGTT |
| | TTTAAAGCAG |
| 451 | ATGATCTTCC CTAATAATAT AGAGGTCACA CGTAAATTCT |
| | ATCCTACAAA |
| 501 | TCAGGAGCTA ATTGAAGATA TTGTTGCAGG TTATCGTAAA |
| | GTCATTCGCG |
| 551 | ATCTTTATGA TGCTGGCTGC CGCTATCTCC AATTAGATGA |
| | CTGTACTCGG |
| 601 | GGAGGTTTAG TAGACCCTCG AGTCTGTTCG TGGTATGGTA |
| | TCGATGAAAA |
| 651 | AGGTCTTCAA GATCTGATTC AACAATATCT TCTGATTAAT |
| | AATCTTGTAA |
| 701 | TTGCAGATCG TCCCGATGAT CTAGTCGTTA ATTTACATGT |
| | ATGCCGTGGG |
| 751 | AACTACCACT CAAAATTCTT TGCTAGTGGT AGTTATGACT |
| | TTATTGCAAA |
| 801 | GCCCCTATTC GAACAAACAA ATGTAGACGG CTACTATTTA |
| | GAGTTTGATC |
| 851 | ATGAGCGTTC TGGAGACTTC TCTCCTCTCA CCTTCATTTC |
| | TGGAGAAAAA |
| 901 | ACTGTCTGCT TAGGTCTTGT TACCAGCAAA ACCCCTACAC |
| | TTGAAAATAA |
| 951 | GGATGAGGTC ATTGCTCGCA TACATCAAGC AGCAGACTAC |
| | CTGCCCTTGG |
| 1001 | AAAGACTCTC TCTAAGTCCA CAGTGTGGTT TTGCTTCATG |
| | TGAAATAGGA |
| 1051 | AATAAATTAA CAGAAGAAGA GCAATGGGCT AAAGTTGCTC |
| | TAGTAAAAGA |
| 1101 | AATTTCCGAA GAAGTTTGGA AATAA |

The PSORT algorithm predicts cytoplasm (0.2171).

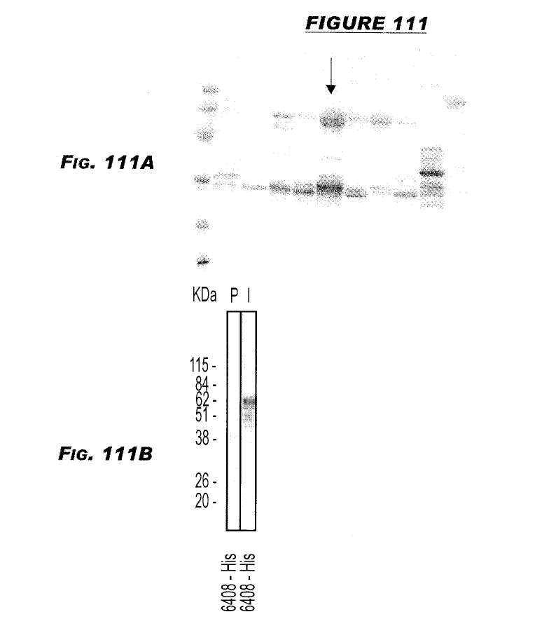

The protein was expressed in E. coli and purified as a GST-fusion product (FIG. 111A) and also as a his-tagged product. The his-tag protein was used to immunise mice, whose sera were used in a Western blot (FIG. 111B) and for FACS analysis.

These experiments show that cp6408 is a surface-exposed and immunoaccessible protein, and that it is a useful immunogen. These properties are not evident from the sequence alone.

### Example 112

The following C. pneumoniae protein (PID 4376430) was expressed <SEQ ID 223; cp6430>:

| | |
|---|---|
| 1 | MKLYSISSDV DTPWIFQLMS KVDSYLFLGG NRIKVVSIVM |
| | QEPNLIIGKV |
| 51 | ENVRISTIVK ILKILSFLIF PLILIALALH YFLHAKYANH |
| | LLVSKILERA |
| 101 | PQYVPIPGRS GDTASHYKLT TLVPVSQKNL QAMGSNPLEV |
| | EAALRTTKPS |
| 151 | FFCVPAKYRQ IIISSHGIRF SLDLEQLADD INLDSVSWPT |
| | EYLNSTMDFC |
| 201 | SKADKRVIQN VQNLRTGTYI NSVGKRSLLK FMLQHLFIDG |
| | ITQENPEALP |
| 251 | NNTSGRLTLF PSVRYIYSHF TPQNPTIWPQ VFFRQGPLDE |
| | DRGGGFEILE |
| 301 | QLQELGVRFP ICPSQGPDNP NFQGFQGIRI YWEDSYQPNK EV* |

The cp6430 nucleotide sequence <SEQ ID 224> is:

| | |
|---|---|
| 1 | ATGAAACTTT ATAGCATCTC TTCAGATGTA GATACACCTT |
| | GGATATTTCA |
| 51 | GCTTATGTCA AAGGTAGATT CTTATCTTTT CTTAGGCGGG |
| | AATAGAATCA |
| 101 | AGGTTCTATC TATAGTTATG CAAGAACCTA ACTTAATTAT |
| | TGGAAAAGTA |
| 151 | GAAAACGTTC GGATCTCCAC AATAGTGAAA ATATTAGAGA |
| | TTTTATCCTT |
| 201 | CTTAATCTTC CCTCTGATTT TAATCGCTTT AGCCCTACAC |
| | TATTTTCTAC |
| 251 | ATGCTAAATA TGCTAATCAC TTACTTGTAT CTAGGATTTT |
| | AGAAAGAGCT |
| 301 | CCTCAGTATG TGCCTATTCC TGGTCGTTCA GGAGACACGG |
| | CGTCTCATTA |
| 351 | TAAATTAACA ACATTGGTTC CAGTATCCCA AAAAAATCTA |
| | CAAGCTATGG |
| 401 | GATCAAATCC TCTAGAAGTT GAAGCGGCTC TTCGAACTAC |
| | AAAACCCTCT |
| 451 | TTTTTCTGTG TACCTGCAAA ATACCGTCAG ATTATAATTT |
| | CAAGTCACGG |
| 501 | CATTCGCTTT TCTTTAGATC TTGAACAACT TGCTGATGAC |
| | ATTAATTTAG |
| 551 | ATTCGGTTTC CTGGCCTACG GAGTATCTTA ACTCTACTAT |
| | GGATTTTTGC |
| 601 | AGCAAGGCAG ATAAACGTGT TATACAGAAT GTACAAAATC |
| | TGCGGACAGG |
| 651 | AACTTACATA ACTTCTGTAG GAAAGCGTAG CCTTTTAAAA |
| | TTCATGTTAC |
| 701 | AGCACCTATT TATTGATGGG ATCACACAAG AAAACCCTGA |
| | AGCCCTTCCT |
| 751 | ACCAATACAT CTGGAAGACT GACTCTATTC CCTAGTGTTC |
| | GTTATATCTA |
| 801 | TTCTCATTTT ACTCCACAAA ATCCTACAAT ATGGCCGCAA |
| | GTCTTTTTCA |
| 851 | GACAAGGTCC TCTAGATGAA GATCGAGGAG GAGGATTTGA |
| | GATCTTAGAG |
| 901 | CAATTACAAG AGTTAGGAGT TAGGTTTCCA ATTTGCCCCT |
| | CTCAAGGACC |
| 951 | AGACAATCCT AATTTTCAAG GTTTTCAAGG GATTCGTATC |
| | TATTGGGAAG |
| 1001 | ATTCCTATCA ACCCAATAAG GAGGTTTAA |

The PSORT algorithm predicts inner membrane (0.5140).

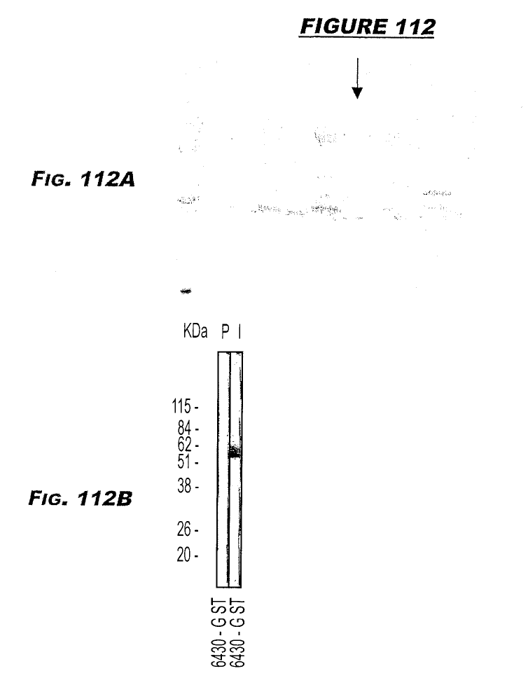

The protein was expressed in E. coli and purified as a GST-fusion product (FIG. 112A). The recombinant protein was used to immunise mice, whose sera were used in a Western blot (FIG. 112B) and for FACS analysis.

These experiments show that cp6430 is a surface-exposed and immunoaccessible protein, and that it is a useful immunogen. These properties are not evident from the sequence alone.

### Example 113

The following C. pneumoniae protein (PID 4376439) was expressed <SEQ ID 225; cp6439>:

| | |
|---|---|
| 1 | MSYDTLFKNL EKEDSVHKIC NEIFALVPRL NTIACTEAII |
| | KNLPKADIHV |
| 51 | HLPGTITPQL AWILGVKNGF LKWSYNSWTN HRLLSPKNPH |
| | KQYSNIFRNF |
| 101 | QDICHEKDPD LSVLQYNILN YDFNSFDRVM ATVQGHRFPP |
| | GGIQNEEDLL |
| 151 | LIFNNYLQQC LDDTIVYTEV QQNIRLAHVL YPSLPEKHAR |
| | MKFYQILYRA |
| 201 | SQTFSKHGIT LRFLNCFNKT FAPQINTQEP AQEAVQWLQE |
| | VDSTFPGLFV |
| 251 | GIQSAGSESA PGACPKRLAS GYRNAYDSGF GCEAHAGEGI |
| | ETRTIFSSAK |
| 301 | VNPEGLIEIT RVTFSSLKRK QPSSLPIRVT CQLG* |

The cp6439 nucleotide sequence <SEQ ID 226> is:

| | |
|---|---|
| 1 | ATGTCTTATG ATACGTTATT CAAGAATCTT GAAAAGGAAG |
| | ATTCTGTACA |
| 51 | TAAGATATGC AATGAGATCT TTGCATTAGT ACCACGACTC |
| | AATACAATCG |
| 101 | CTTGCACCGA AGCTATCATC AAAAACCTCC CCAAAGCAGA |
| | TATCCATGTA |
| 151 | CACCTTCCTG GGACCATAAC ACCTCAATTA GCTTGGATTT |
| | TAGGTGTGAA |
| 201 | AAATGGGTTC TTAAAATGGT CTTATAATTC TTGGACCAAT |
| | CATCGATTAC |
| 251 | TTTCTCCTAA GAATCCTCAT AAACAATACT CCAATATTTT |
| | CCGAAACTTT |
| 301 | CAAGATATCT GTCACGAAAA GGATCCGGAT TTAAGTGTAT |
| | TACAATATAA |
| 351 | TATCTTAAAT TACGATTTTA ATAGCTTTGA TAGAGTGATG |
| | GCTACAGTAC |
| 401 | AAGGACATCG CTTTCCTCCT GGAGGAATCC AAAATGAAGA |
| | AGACCTTCTT |
| 451 | CTCATTTTCA ATAACTATCT CCAGCAATGT CTGGACGATA |
| | CTATCGTGTA |
| 501 | TACTGAAGTA CAACAAAATA TCCGCCTTGC CCATGTTTTG |
| | TATCCTTCAT |
| 551 | TACCTGAAAA GCACGCGCGT ATGAAGTTTT ATCAAATCTT |
| | GTATCGTGCT |
| 601 | TCGCAAACGT TTTCAAAACA CGGGATTACT TTACGATTTT |
| | TAAACTGCTT |
| 651 | CAATAAAACA TTTGCTCCAC AAATAAACAC ACAAGAACCT |
| | GCCCAAGAAG |
| 701 | CTGTTCAATG GCTCCAAGAG GTTGATTCTA CATTTCCTGG |
| | TCTATTTGTA |
| 751 | GGGATACAAT CCGCAGGATC AGAATCTGCG CCCGGAGCCT |
| | GTCCTAAGCG |
| 801 | ATTAGCTTCT GGATATAGAA ATGCTTATGA CGCAGGGTTT |
| | GGTTGTGAAG |
| 851 | CTCATGCTGG AGAAGGCATA GAGACCCGGA CTATTTTTTC |
| | GTCAGCTAAG |
| 901 | GTAAATCCAG AGGGATTGAT CGAGATAACC CGAGTGACTT |
| | TCTCGTCTCT |
| 951 | TAAACGAAAA CAGCCATCTA GTTTACCCAT AAGAGTTACT |
| | TGCCAGTTAG |
| 1001 | GATAA |

The PSORT algorithm predicts cytoplasm (0.1628).

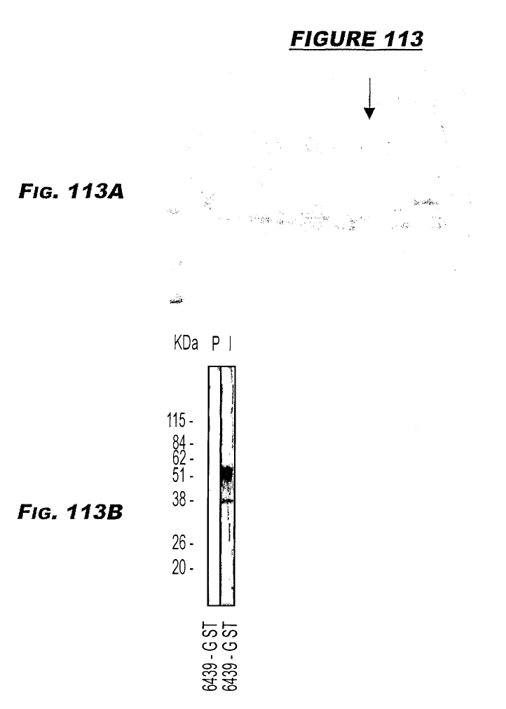

The protein was expressed in E. coli and purified as a GST-fusion product (FIG. 113A). The recombinant protein was used to immunise mice, whose sera were used in a Western blot (FIG. 113B) and for FACS analysis.

These experiments show that cp6439 is a surface-exposed and immunoaccessible protein, and that it is a useful immunogen. These properties are not evident from the sequence alone.

### Example 114

The following C. pneumoniae protein (PID 4376440) was expressed <SEQ ID 227; cp6440>:

| | |
|---|---|
| 1 | LQSARRHLNT IFLLDFGSQY TYVLAKQVRK LFVYCEVLPQ |
| | NISVQCLKER |
| 51 | APLGIILSGG PHSVYENKAP HLDPEIYKLG IPILAICYGM |
| | QLMARDFGGT |
| 101 | VSPGVGEFGY TPIHLYPCEL FKHIVDCESL DTEIRMSHRD |
| | HVTTIPEGFN |
| 151 | VIASTSQCSI SGIENTKQRL YGLQFHPEVS DSTPTGNKIL |
| | ETFVQEICSA |
| 201 | PTLWNPLYIQ QDLVSKIQDT VIEVFDEVAQ SLDVQWLAQG |
| | TIYSDVIESS |
| 251 | RSGHASEVIK SHHNVGGLPK NLKLKLVEPL RYLFKDEVRI |
| | LGEALGLSSY |
| 301 | LLDRHPFPGP GLTIRVIGEI LPEYLAILRR ADLIFIEELR |
| | KAKLYDKISQ |
| 351 | AFALFLPIKS VSVKGDCRSY GYTIALRAVE STDFMTGRWA |
| | YLPCDVLSSC |
| 401 | SRRIINEIPE VSRVVYDISD KPPATIEWE* |

The cp6440 nucleotide sequence <SEQ ID 228> is:

| | |
|---|---|
| 1 | TTGCAGAGTG CAAGGAGACA TTTGAACACC ATATTTATTC |
| | TAGATTTTGG |
| 51 | ATCTCAATAT ACTTATGTAT TAGCAAAGCA AGTGCGGAAG |
| | TTATTTGTAT |
| 101 | ATTGCGAAGT TCTTCCCTGG AATATCTCTG TGCAATGTTT |
| | AAAAGAAAGA |
| 151 | GCGCCTTTGG GGATCATTCT CTCAGGAGGT CCTCACTCTG |
| | TCTATGAAAA |
| 201 | CAAGGCTCCA CATTTAGATC CTGAAATCTA TAAACTTGGC |
| | ATTCCAATTC |
| 251 | TAGCTATTTG CTATGGCATG CAGCTTATGG CTAGAGATTT |
| | TGGAGGGACT |
| 301 | GTAAGCCCTG GTGTAGGAGA ATTTGGATAT ACGCCCATCC |
| | ATCTGTATCC |
| 351 | TTGTGAGCTC TTCAAACACA TCGTCGACTG CGAATCTCTA |
| | GACACAGAGA |
| 401 | TTCGGATGAG CCATCGGGAT CATGTTAGCA CAATTCCTGA |
| | AGGATTTAAT |
| 451 | GTAATCGCAT CCACCTCACA ATGCTCGATC TCAGGAATAG |
| | AAAATACCAA |
| 501 | ACAACGGTTG TACGGGCTGC AATTTCATCC CGAGGTTTCT |
| | GACTCCACTC |
| 551 | CAACGGGAAA TAAGATTCTA GAAACTTTTG TTCAAGAGAT |
| | CTGTTCTGCT |
| 601 | CCCACACTAT GGAATCCCTT GTATATTCAG CAAGACCTTG |
| | TAAGTAAAAT |
| 651 | TCAAGATACC GTTATTGAAG TATTTGATGA AGTCGCTCAG |
| | TCATTAGACG |
| 701 | TACAATGGTT AGCTCAAGGA ACCATCTACT CAGATGTTAT |
| | TGAGTCCTCA |
| 751 | CGCTCTGGAC ATGCCTCCGA AGTAATAAAA TCACATCATA |
| | ATGTAGGGGG |
| 801 | GCTTCCAAAA AATCTTAAGC TGAAGTTAGT CGAGCCCTTA |
| | CGTTATTTAT |
| 851 | TTAAAGATGA AGTTCGAATT TTAGGAGAAG CCCTAGGACT |
| | TTCTAGCTAT |
| 901 | CTCTTGGACA GGCATCCTTT TCCTGGACCT GGCTTGACAA |
| | TTCGTGTGAT |
| 951 | TGGAGAGATC CTTCCTGAAT ATCTAGCCAT TTTACGACGG |
| | GCGGACCTCA |
| 1001 | TCTTTATAGA AGAGCTTAGG AAAGCAAAAC TCTACGATAA |
| | AATAAGCCAA |
| 1051 | GCCTTTGCTC TATTTCTTCC TATAAAATCA GTATCTGTAA |
| | AAGGAGATTG |
| 1101 | TAGAAGCTAT GGTTATACCA TAGCATTACG TGCTGTAGAA |
| | TCTACAGATT |
| 1151 | TCATGACAGG ACGATGGGCC TACCTTCCAT GCGATGTTCT |
| | CAGTTCTTGC |
| 1201 | TCATCGCGAA TTATTAATGA AATACCCGAG GTAAGCCGAG |
| | TGGTCTATGA |
| 1251 | TATTTCTGAC AAGCCACCAG CAACTATAGA ATGGGAATAG |

The PSORT algorithm predicts cytoplasm (0.0481).

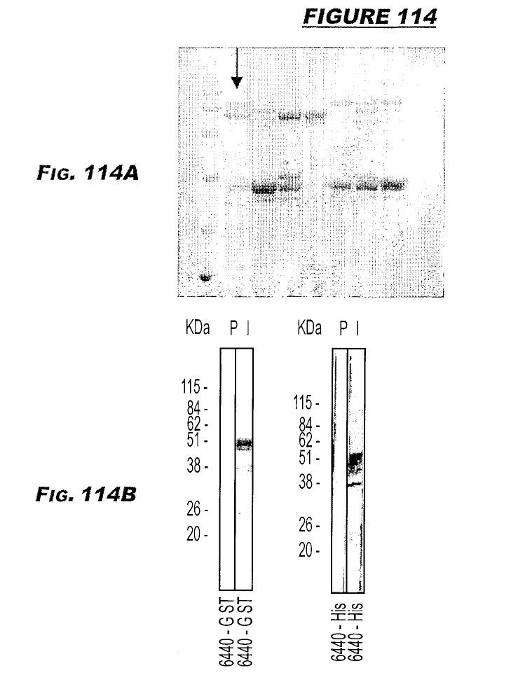

The protein was expressed in E. coli and purified as a GST-fusion product (FIG. 114A) and also as a his-tagged product. The recombinant proteins were used to immunise mice, whose sera were used in a Western blot (FIG. 114B) and for FACS analysis.

These experiments show that cp6440 is a surface-exposed and immunoaccessible protein, and that it is a useful immunogen. These properties are not evident from the sequence alone.

### Example 115

The following C. pneumoniae protein (PID 4376475) was expressed <SEQ ID 229; cp6475>:

| | |
|---|---|
| 1 | MNTYTFSPTL QKSFSLFLLE KLDSYFFFGG TRTQILVITP |
| | TNIRLAAKKR |
| 51 | GCKVSTIEKI IKILSFILLP LVIIAFILRY FLHKKFDKQF |
| | LCIPKVISNE |
| 101 | DEALLGSRPQ AVEKAVREIS PAFFSIPRKY QLIRIDTPKD |
| | DAPSILFPIG |
| 151 | IEIILKDLCI DTLKQSNLFL KREMDFLGHP EEKALFDSIC |
| | SIEKDQEWMS |
| 201 | LESKKLLITH FLKYLFVSGI EQLNPGFNPE NGRGYFSEIS |
| | TAKIHFHQHG |
| 251 | RYGPIRSSGP IMKEI* |

The cp6475 nucleotide sequence <SEQ ID 230> is:

| | |
|---|---|
| 1 | ATGAATACCT ATACCTTCTC TCCTACACTT CAGAAAAGCT |
| | TCAGCCTATT |
| 51 | TCTTTTAGAA AAATTAGACT CTTACTTTTT CTTTGGAGGG |
| | ACTCGTACAC |
| 101 | AAATCTTAGT CATCACACCA ACCAATATTA GATTAGCAGC |
| | TAAAAAAAGA |
| 151 | GGGTGTAAGG TTTCTACTAT AGAAAAGATA ATCAAGATCC |
| | TCTCTTTTAT |
| 201 | CCTGCTGCCC CTAGTTATCA TTGCCTTTAT ACTTCGCTAT |
| | TTCTTACATA |
| 251 | AGAAATTCGA TAAACAGTTC TTGTGTATCC CAAAAGTCAT |
| | TTCTAACGAA |
| 301 | GACGAAGCTC TTCTTGGATC TAGACCACAA GCAGTTGAAA |
| | AAGCAGTTCG |
| 351 | AGAAATATCT CCAGCCTTCT TCTCTATACC AAGAAAATAC |
| | CAACTTATTA |
| 401 | GAATCGACAC TCCTAAAGAT GACGCTCCCT CAATCCTTTT |
| | CCCTATAGGC |
| 451 | ATAGAGATCA TTCTCAAAGA TTTATGTATT GATACACTCA |
| | AGCAATCTAA |
| 501 | TCTTTTCCTT AAAAGAGAAA TGGATTTCTT AGGTCATCCA |
| | GAAGAAAAAG |
| 551 | CATTATTCGA CTCGATATGT TCTATAGAAA AAGATCAAGA |
| | ATGGATGAGC |
| 601 | TTGGAAAGTA AAAAACTTTT AATCACGCAC TTCCTAAAGT |
| | ATCTCTTTGT |
| 651 | CTCTGGAATC GAACAACTAA ATCCAGGCTT TAACCCAGAG |
| | AATGGGCGTG |
| 701 | GGTATTTTTC AGAAATAAGT ACAGCAAAGA TCCATTTTCA |
| | TCAGCACGGT |
| 751 | CGATATGGGC CAATCCGTTC TTCGGGACCC ATCATGAAGG |
| | AAATATAA |

The PSORT algorithm predicts inner membrane (0.5373).

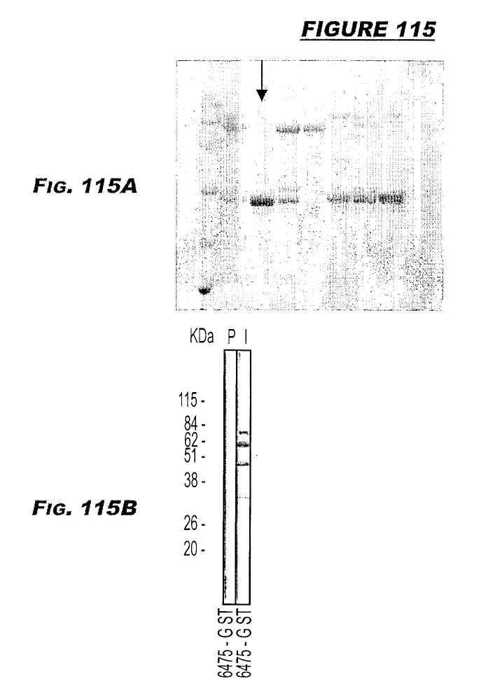

The protein was expressed in E. coli and purified as a GST-fusion product (FIG. 115A). The recombinant protein was used to immunise mice, whose sera were used in a Western blot (FIG. 115B) and for FACS analysis.

These experiments show that cp6475 is a surface-exposed and immunoaccessible protein, and that it is a useful immunogen. These properties are not evident from the sequence alone.

### Example 116

The following C. pneumoniae protein (PID 4376482) was expressed <SEQ ID 231; cp6482>:

| | |
|---|---|
| 1 | MLVELEALKR EFAHLKDQKP TSDQEITSLY QCLDHLEFVL |
| | LGLGQDKFLK |
| 51 | ATEDEDVLFE SQKAIDAWNA LLTKARDVLG LGDIGAIYQT |
| | IEFLGAYLSK |
| 101 | VNRRAFCIAS EIHFLKTAIR DLNAYYLLDF RWPLCKIEEF |
| | VDWGNDCVEI |
| 151 | AKRKLCTFEK ETKELNESLL REEHAMEKCS IQDLQRKLSD |
| | IIIELHDVSL |
| 201 | FCFSKTPSQE EYQKDCLYQS RLRYLLLLYE YTLLCKTSTD |
| | FQEQARAKEE |
| 251 | FIREKFSLLE LEKGIKQTKE LEFAIAKSKL ERGCLVMRKY |
| | EAAAKHSLDS |
| 301 | MFEEETVKSP RKDTE* |

The cp6482 nucleotide sequence <SEQ ID 232> is:

| | |
|---|---|
| 1 | ATGCTAGTAG AGTTAGAGGC TCTTAAAAGA GAGTTTGCGC |
| | ATTTAAAAGA |
| 51 | CCAGAAGCCG ACAAGTGACC AAGAGATCAC TTCACTTTAT |
| | CAATGTTTGG |
| 101 | ATCATCTTGA ATTCGTTTTA CTCGGGCTGG GCCAGGACAA |
| | ATTTTTAAAG |
| 151 | GCTACGGAAG ATGAAGATGT GCTTTTTGAG TCTCAAAAAG |
| | CAATCGATGC |
| 201 | GTGGAATGCT TTATTGACAA AAGCCAGAGA TGTTTTAGGT |
| | CTTGGGGACA |
| 251 | TAGGTCCTAT CTATCAGACT ATAGAATTCT TGGGTGCCTA |
| | TTTATCAAAA |
| 301 | GTGAATCGGA GGGCTTTTTG TATTGCTTCG GAGATACATT |
| | TTCTAAAAAC |
| 351 | AGCAATCCGA GATTTGAATG CATATTACCT GTTAGATTTT |
| | AGATGGCCTC |
| 401 | TTTCCAAGAT AGAAGAGTTT GTGGATTGGG GGAATGATTG |
| | TGTTGAAATA |
| 451 | GCAAAGAGGA AGCTATGCAC TTTTGAAAAA GAAACCAAGG |
| | AGCTCAATGA |
| 501 | GAGCCTTCTT AGAGAGGAGC ATGCGATGGA GAAATGCTCG |
| | ATTCAAGATC |
| 551 | TGCAAAGGAA ACTTAGCGAC ATTATTATTG AATTGCATGA |
| | TGTTTCTCTT |
| 601 | TTTTGTTTTT CTAAGACTCC CAGTCAAGAG GAGTATCAAA |
| | AGGATTGTTT |
| 651 | GTATCAATCA CGATTGAGGT ACTTATTGTT GCTGTATGAG |
| | TATACATTGT |
| 701 | TATGTAAGAC ATCCACAGAT TTTCAAGAGC AGGCTAGGGC |
| | TAAAGAGGAG |
| 751 | TTCATTAGGG AGAAATTCAG CCTTCTAGAG CTCGAAAAGG |
| | GAATAAAACA |
| 801 | ACCTAAAGAG CTTGAGTTTG CAGTTGCTAA AAGTAAGTTA |
| | GAACGGGGCT |
| 851 | GTTTAGTTAT GAGGAAGTAT GAAGCTGCCG CTAAACATAG |
| | TTTAGATTCT |
| 901 | ATGTTCGAAG AAGAAACTGT GAAGTCGCCG CGGAAAGACA |
| | CAGAATAA |

The PSORT algorithm predicts cytoplasm (0.4607).

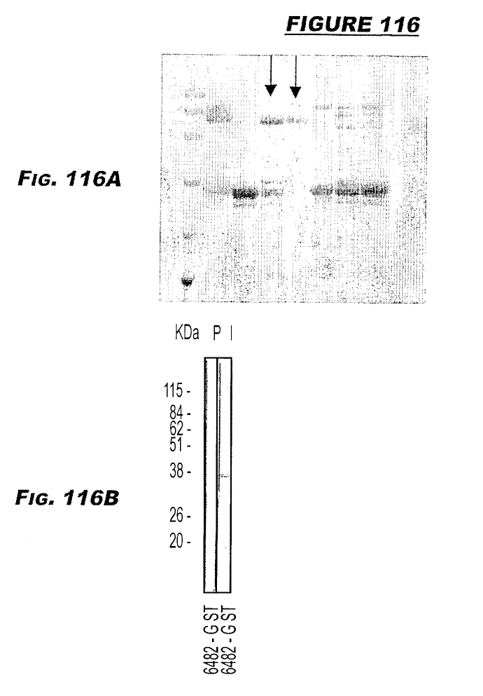

The protein was expressed in E. coli and purified as a GST-fusion product (FIG. 116A). The recombinant protein was used to immunise mice, whose sera were used in a Western blot (FIG. 116B) and for FACS analysis.

These experiments show that cp6482 is a surface-exposed and immunoaccessible protein, and that it is a useful immunogen. These properties are not evident from the sequence alone.

### Example 117

The following C. pneumoniae protein (PID 4376486) was expressed <SEQ ID 233; cp6486>:

| | |
|---|---|
| 1 | VVVVALFILG IFFLSGSLAF LVHTSCGVLL GAALPILCIG |
| | LVLLAVALIV |
| 51 | FLCHKHKTRQ DLDYYDQDLD SLVIHKKEIP NDISELRVTF |
| | EKLQNLFQFH |
| 101 | TKDFSDLSQE LQGKFINCME KWLTLEDEVT KFLIVRDRFL |
| | ETRRNFTTFG |
| 151 | EQVKGIQSNI FDLHEEKSSL YLELYRLRKD LQVLLNFFLL |
| | PPGILKVDYD |
| 201 | EIEAIKGLFI RLTSRLDKLD VKAQERKKFI NEMSREFKEV |
| | EKAFDIVDRA |
| 251 | TKKLMDRAKK ESPARLFMGR TESLLEMKKN EEALKNQGLD |
| | PENLSHPELF |
| 301 | SPYQQLLILN YLNSEIVLHH YEFLISGTVT SGLTLEECEN |
| | RMRAASTGLN |
| 351 | ALLVRKLQFR GAIKSAYFEK LTEIEKELRS LQDVIKSLEL |
| | ELIHKIKDIV |
| 401 | TEET* |

The cp6486 nucleotide sequence <SEQ ID 234> is:

| | |
|---|---|
| 1 | GTGGTGGTTG TCGCTTTATT TATCCTTGGG ATTTTCTTTT |
| | TATCTGGTTC |
| 51 | TCTTCCATTC CTTGTTCATA CGTCTTGCGG AGTTCTTTTA |
| | GGAGCGGCGC |
| 101 | TTCCCATACT TTGCATAGGT CTTGTTTTAT TGGCTGTAGC |
| | TCTTATTGTT |
| 151 | TTCTTATGTC ACAAACACAA GACTCGTCAA GATTTAGATT |
| | ATTATGATCA |
| 201 | AGATTTAGAT TCTTTGGTGA TTCATAAGAA AGAGATCCCC |
| | AATGACATCT |
| 251 | CTGAGTTGCG GGTAACATTT GAAAAGTTGC AAAATCTGTT |
| | TCAGTTCCAT |
| 301 | ACGAAAGATT TCTCTGATCT AAGCCAAGAG CTTCAGGGTA |
| | AATTTATCAA |
| 351 | TTGCATGGAG AAATGGCTAA CTTTAGAAGA CGAAGTGACT |
| | AAATTTCTTA |
| 401 | TTGTTCGAGA TAGATTTTTA GAAACCAGAA GAAATTTTAC |
| | CACTTTTGGA |
| 451 | GAACAGGTTA AAGGGATCCA AAGCAATATT TTTGATTTGC |
| | ATGAGGAAAA |
| 501 | GTCTTCATTA TATTTAGAAT TGTATAGGCT TAGGAAAGAC |
| | CTCCAAGTTC |
| 551 | TATTAAATTT TTTTCTGCTC CCCCCAGGTA TACTCAAGGT |
| | AGATTATGAT |
| 601 | GAAATTGAGG CTATCAAAGG TCTGTTTATA AGATTAACCT |
| | CTAGATTAGA |
| 651 | TAAGCTTGAT GTGAAAGCTC AGGAACGTAA GAAGTTCATT |
| | AATGAAATGA |
| 701 | GTAGGGAATT TAAAGAAGTA GAGAAAGCTT TTGATATTGT |
| | CGATAGGGCA |
| 751 | ACAAAAAAGC TTATGGATAG AGCCAAGAAA GAAAGTCCGG |
| | CACGTCTTTT |
| 801 | CATGGGTAGA ACTGAGTCTC TCTTAGAAAT GAAAAAAAAT |
| | GAAGAAGCCC |
| 851 | TTAAAAATCA GGGGCTAGAT CCTGAAAATC TTTCCCATCC |
| | TGAACTTTTT |
| 901 | AGTCCGTATC AACAGCTTTT AATTTTGAAT TATTTAAATA |
| | GCGAAATAGT |
| 951 | TCTGCATCAT TATGAGTTCC TTATTTCTGG AACAGTAACT |
| | TCTGGCCTAA |
| 1001 | CTCTTGAAGA ATGTGAAAAT CGAATGAGGG CGGCTTCTAC |
| | TGGGTTGAAC |
| 1051 | GCCCTTCTGG TGCGTAAGCT CCAGTTCAGA GGTGCTATAA |
| | AATCTGCGTA |
| 1101 | TTTTGAAAAA CTCACAGAGA TTGAAAAAGA GTTACGATCA |
| | CTTCAAGACG |
| 1151 | TAATAAAGTC ATTGGAACTA GAACTGATCC ATAAGATAAA |
| | AGATATAGTG |
| 1201 | ACAGAAGAAA CTTAG |

The PSORT algorithm predicts inner membrane (0.7474).

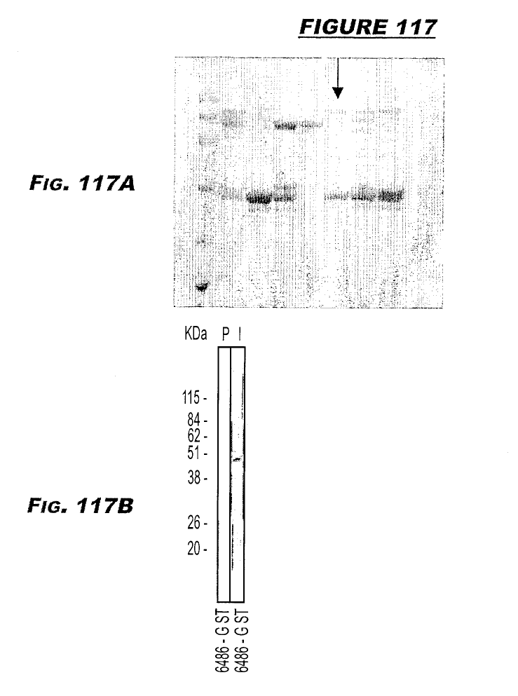

The protein was expressed in E. coli and purified as a GST-fusion product (FIG. 117A). The recombinant protein was used to immunise mice, whose sera were used in a Western blot (FIG. 117B) and for FACS analysis.

These experiments show that cp6486 is a surface-exposed and immunoaccessible protein, and that it is a useful immunogen. These properties are not evident from the sequence alone.

### Example 118

The following C. pneumoniae protein (PID 4376526) was expressed <SEQ ID 235; cp6526>:

| | |
|---|---|
| 1 | MSPFKKIVNR LLCYISFQKE SRTLPIIIRE PRMTTKSLGS |
| | FNSVISKNKI |
| 51 | HFISLGCSRN LVDSEVMLGI LLKAGYESTN EIEDADYLIL |
| | NTCAFLKSAR |
| 101 | DEAKDYLDHL IDVKKENAKI IVTGCMTSNH KDELKPWMSH |
| | IHYLLGSGDV |
| 151 | ENILSAIESR ESGEKISAKS YIEMGEVPRQ LSTPKHYAYL |
| | KVAEGCRKRC |
| 201 | AFCIIPSIKG KLRSKPLDQI LKEFRILVNK SVKEIILIAQ |
| | DLGDYGDDLS |
| 251 | TDRSSQLESL LHELLKEPGD YWLRMLYLYP DEVSDGIIDL |
| | MQSNPKLLPY |
| 301 | VDIPLQHIND RILKQMRRTT SREQILGFLE KLRAKVPQVY |
| | IRSSVIVGFP |
| 351 | GETQEEFQEL ADFIGEGWID NLGIFLYSQE ANTPAAELPD |
| | QIPEKVKESR |
| 401 | LKILSQIQKR NVDKHNQKLI GEKIEAVIDN YHPETNLLLT |
| | ARFYGQAPEV |
| 451 | CPCIIVNEAK LVSHFGERCF IEITGTAGYD LVGRVVKKSQ |
| | NQALLKTSKA |
| 501 | * |

The cp6526 nucleotide sequence <SEQ ID 236> is:

| | |
|---|---|
| 1 | ATGAGTCCTT TTAAGAAAAT AGTAAATCGC TTACTATGCT |
| | ATATTTCTTT |
| 51 | TCAAAAAGAA TCAAGAACTC TCCCAATCAT TATTAGAGAA |
| | CCTAGGATGA |
| 101 | CAACAAAAAG TTTAGGATCT TTCAATTCAG TTATTTCCAA |
| | AAATAAAATT |
| 151 | CATTTTATTA GTTTGGGATG CTCTCGGAAC CTTGTAGATA |
| | GCGAAGTCAT |
| 201 | GCTAGGCATT CTTCTTAAGG CAGGTTACGA GTCTACTAAT |
| | GAAATTGAAG |
| 251 | ATGCTGACTA TTTAATTTTA AATACCTGTG CGTTTTTAAA |
| | AAGTGCTAGA |
| 301 | GATGAAGCTA AAGATTATCT AGACCATCTA ATTGATGTAA |
| | AAAAAGAGAA |
| 351 | CGCTAAAATT ATTGTAACTG GATGCATGAC TTCCAACCAC |
| | AAAGATGAGC |
| 401 | TTAAACCCTG GATGTCACAC ATCCATTACC TACTAGGTTC |
| | TGGGGATGTT |
| 451 | GAGAATATTC TTTCTGCTAT TGAGTCTCGT GAATCTGGAG |
| | AAAAAATCTC |
| 501 | TGCAAAGAGT TACATTGAGA TGGGAGAAGT TCCAAGACAG |
| | CTTTCCACAC |
| 551 | CAAAACACTA TGCCTATTTA AAAGTTGCTG AGGGCTGTAG |
| | AAAACGTTGT |
| 601 | GCTTTTTGTA TTATTCCTTC CATTAAAGGA AAGCTCCGCA |
| | GCAAACCTCT |
| 651 | GGATCAAATT CTTAAAGAAT TCCGCATCCT TGTAAACAAG |
| | AGTGTGAAAG |
| 701 | AGATTATATT GATAGCTCAA GACCTAGGAG ATTATGGAAA |
| | GGATCTCTCT |
| 751 | ACAGACCGCA GTTCGCAGCT AGAATCACTA TTACATGAGT |
| | TACTGAAAGA |
| 801 | GCCTGGTGAT TATTGGCTGC GGATGTTGTA TTTATATCCT |
| | GATGAAGTGA |
| 851 | GTGATGGCAT TATAGATCTT ATGCAATCTA ATCCCAAACT |
| | TCTTCCCTAT |
| 901 | GTAGATATTC CCTTACAGCA CATTAACGAC CGTATTTTAA |
| | AGCAAATGCG |
| 951 | AAGAACGACT TCTAGGGAGC AAATCCTAGG ATTCCTAGAA |
| | AAATTACGTG |
| 1001 | CCAAGGTTCC TCAGGTCTAT ATCCGTTCTT CTGTTATTGT |
| | GGGTTTCCCC |
| 1051 | GGTGAAACTC AGGAAGAATT CCAGGAGTTA GCTGATTTTA |
| | TTGGTGAGGG |
| 1101 | TTGGATTGAT AATCTCGGAA TTTTCTTGTA CTCTCAAGAA |
| | GCGAATACCC |
| 1151 | CGGCAGCAGA ACTCCCTGAC CAGATACCAG AAAAAGTTAA |
| | AGAATCGAGG |
| 1201 | TTGAAAATTC TATCTCAAAT TCAGAAACGC AATGTGGATA |
| | AACATAATCA |
| 1251 | GAAGCTCATT GGGGAAAAAA TAGAAGCAGT TATTGATAAC |
| | TATCATCCTG |
| 1301 | AAACGAATCT TTTACTCACT GCAAGGTTCT ATGGACAAGC |
| | TCCTGAAGTG |
| 1351 | GACCCTTGTA TTATTGTAAA TGAGGCGAAG CTTGTTTCTC |
| | ATTTTGGAGA |
| 1401 | AAGATGCTTT ATAGAAATCA CAGGGACTGC TGGTTACGAC |
| | CTTGTAGGGC |
| 1451 | GTGTTGTAAA AAAATCTCAG AACCAAGCTT TGCTAAAAAC |
| | TAGCAAAGCT |
| 1501 | TAG |

The PSORT algorithm predicts cytoplasm (0.1296).

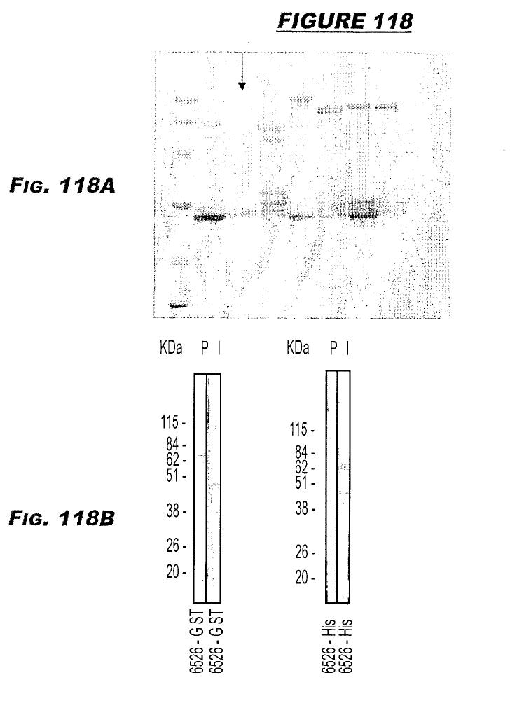

The protein was expressed in E. coli and purified as a GST-fusion product (FIG. 118A) and also as a his-tagged product. The recombinant proteins were used to immunise mice, whose sera were used in a Western blot (FIG. 118B) and for FACS analysis.

These experiments show that cp6526 is a surface-exposed and immunoaccessible protein, and that it is a useful immunogen. These properties are not evident from the sequence alone.

### Example 119

The following C. pneumoniae protein (PID 4376528) was expressed <SEQ ID 237; cp6528>:

| | |
|---|---|
| 1 | MKNNINNNEC YFKLDSTVDG DLLAANLKTF DTQAQGISST |
| | ETFSVQGNAT |
| 51 | FKDQVSATGL TSGTTYNLNA QNFTSSQISI DFKNNRLSNC |
| | ALPKFDCDPV |
| 101 | PANYVRSPEY FFCSKPLIGD FDFNSGESYL PLTGSEYTLY |
| | QSRNVNSIFR |
| 151 | FIGWKQSTRE LTVGGNTAIQ FLAAGTYIVS FTVGKRWGWN |
| | NGWGGAIYIN |
| 201 | NGLGQVQCES TIYSGGGYAT IGTLGTSIYR ASVDVAPNPN |
| | DPNASDRYRA |
| 251 | GIFYLSNGGS SAGIGNYSFS LLYYPDDRG* |

The cp6528 nucleotide sequence <SEQ ID 238> is:

| | |
|---|---|
| 1 | ATGAAAAACA ATATTAATAA TAATGAGTGC TATTTTAAAT |
| | TAGACTCAAC |
| 51 | TGTAGATGGT GATTTGTTAG CAGCCAATCT CAAGACCTTT |
| | GATACACAGG |
| 101 | CCCAAGGAAT CTCATCGACT GAAACATTTT CTGTTCAGGG |
| | GAATGCAACA |
| 151 | TTTAAAGATC AAGTTTCAGC AACTGGATTA ACTTCAGGAA |
| | CTACTTATAA |
| 201 | TTTAAATGCA CAAAACTTTA CTTCCTCCCA AATCTCTATA |
| | GATTTTAAAA |
| 251 | ATAATCGTCT GAGTAATTGT GCATTGCCAA AAGAAGACTG |
| | CGATCCGGTG |
| 301 | CCAGCGAATT ATGTTCGTTC TCCCGAATAT TTTTTCTGTT |
| | CCAAGCCTCT |
| 351 | GATCGGAGAT TTTGATTTTA ACTCAGGGGA ATCTTATTTG |
| | CCTCTGACTG |
| 401 | GTTCGGAATA TACTCTATAT CAGTCACGTA ATGTAAATAG |
| | TATATTTCGT |
| 451 | TTTATAGGAT GGAAGCAAAG TACACGAGAA TTAACTGTAG |
| | GGGGAAATAC |
| 501 | TGCGATACAA TTTCTTGCAG CAGGAACCTA TATCGTTTCA |
| | TTTACTGTTG |
| 551 | GTAAACGGTG GGGATGGAAT AATGGTTGGG GAGGAGCCAT |
| | TTATATCAAT |
| 601 | AATGGTTTAG GACAAGTCCA ATGTGAAAGC ACGATTTATA |
| | GTGGTGGAGG |
| 651 | GTATGCAACA ATAGGTACAC TGGGGACCTC AATATATAGA |
| | GCCTCTGTAG |
| 701 | ATGTAGCTCC TAATCCTAAT GATCCGAATG CTTCGGATCG |
| | CTATAGAGCG |
| 751 | GGTATTTTCT ATCTCAGTAA CGGTGGTTCT AGTGCAGGTA |
| | TAGGGAATTA |
| 801 | CTCCTTTTCT CTTCTCTATT ATCCGGACGA TAGAGGGTAG |

The PSORT algorithm predicts cytoplasm (0.1668).

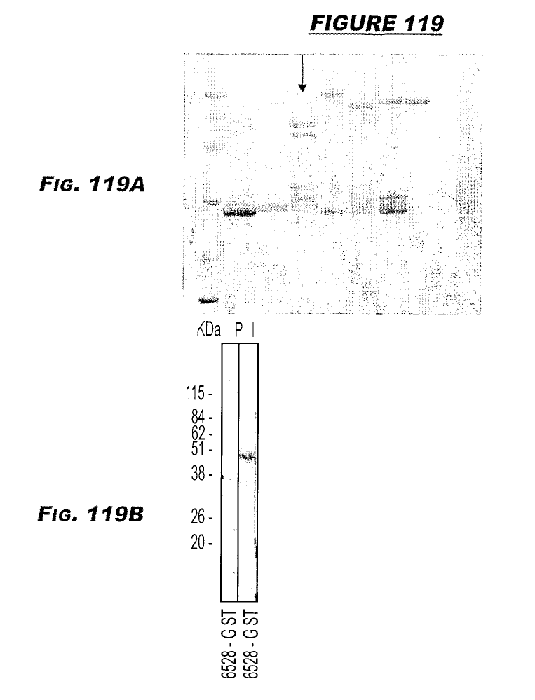

The protein was expressed in E. coli and purified as a GST-fusion product (FIG. 119A). The recombinant protein was used to immunise mice, whose sera were used in a Western blot (FIG. 119B) and for FACS analysis.

These experiments show that cp6528 is a surface-exposed and immunoaccessible protein, and that it is a useful immunogen. These properties are not evident from the sequence alone.

### Example 120

The following C. pneumoniae protein (PID 4376627) was expressed <SEQ ID 239; cp6627>:

| | |
|---|---|
| 1 | MKCSPLTLVP HIFLKNDCEC HRSCSLKIRT IARLILGLVL |
| | ALVSALSFVF |
| 51 | LAAPISYAIG GTLALAAIVI LIITLVVALL AKSKVLPIPN |
| | ELQKIIYNRY |
| 101 | PKEVFYFVKT HSLTVNELKI FINCWKSGTD LPPNLHKKAE |
| | AFGIDILKSI |
| 151 | DLTLFPEFEE ILLQNCPLYW LSHFIDKTES VAGEIGLNKT |
| | QKVYGLLGPL |
| 201 | AFHKGYTTIF HSYTRPLLTL ISESQYKFLY SKASKNQWDS |
| | PSVKKTCEEI |
| 251 | FKELPHNMIF RKDVQGISQF LELFFSHGIT WEQAQMIQLI |
| | NPDNWKMLCQ |
| 301 | FDKAGGHCSM ATFGGFLNTE TNMFDPVSSN YEPTVNFMTW |
| | KELKVLLEKV |
| 351 | KESPMHPASA LVQKICVNTT HHQNLLKRWQ FVRNTSSQWT |
| | SSLPQYAFHA |
| 401 | QTYKLEKKIE SSLPIRSSL* |

The cp6627 nucleotide sequence <SEQ ID 240> is:

| | |
|---|---|
| 1 | ATGAAGTGTA GTCCTTTAAC ACTAGTTCCC CATATATTTT |
| | TAAAAAATGA |
| 51 | CTGCGAATGT CATAGATCTT GTTCTTTAAA AATTAGGACA |
| | ATTGCCCGAC |
| 101 | TCATTCTTGG GCTTGTTCTA GCTCTTGTTA GCGCACTTTC |
| | TTTTGTTTTC |
| 151 | CTTGCTGCGC CGATTAGCTA TGCTATTGGA GGAACTTTAG |
| | CTTTAGCCGC |
| 201 | TATCCTAATC TTGATTATAA CGCTAGTCGT ACCACTCCTA |
| | GCTAAATCAA |
| 251 | AGGTTCTGCC CATCCCCAAC GAACTTCAGA AGATTATTTA |
| | CAATCGCTAT |
| 301 | CCTAAAGAAG TCTTTTATTT CGTGAAAACA CACTCCCTGA |
| | CTGTTAACGA |
| 351 | ATTAAAAATA TTTATTAATT GCTGGAAAAG CGGTACAGAC |
| | CTGCCTCCGA |
| 401 | ATTTACATAA AAAAGCAGAG GCTTTCGGGA TCCATATTCT |
| | AAAATCTATA |
| 451 | GATTTAACCC TGTTTCCAGA GTTCGAAGAG ATTCTTCTTC |
| | AAAACTGCCC |
| 501 | GTTATACTGG CTCTCCCATT TTATAGACAA AACTGAATCT |
| | GTTGCTGGGG |
| 551 | AAATCGGATT AAATAAAACA CAAAAAGTTT ATGGTTTACT |
| | TGGGCCCTTA |
| 601 | GCGTTTCATA AAGGATATAC AACTATTTTC CACTCTTATA |
| | CACGCCCTCT |
| 651 | ACTAACATTA ATCTCAGAAT CACAGTATAA GTTCCTATAT |
| | AGTAAAGCGT |
| 701 | CTAAGAATCA ATGGGATTCT CCTTCTGTGA AAAAAACCTG |
| | CGAAGAAATA |
| 751 | TTCAAGGAAC TCCCCCACAA TATGATTTTC CGGAAGGATG |
| | TTCAAGGAAT |
| 801 | CTCACAATTC TTATTTCTTT TCTTTTCTCA TGGTATCACT |
| | TGGGAACAGG |
| 851 | CTCAGATGAT TCAACTTATA AATCCTGATA ATTGGAAAAT |
| | GTTGTGTCAG |
| 901 | TTTGATAAAG CAGGAGGCCA CTGTTCCATG GCAACATTTG |
| | GAGGCTTTTT |
| 951 | GAATACTGAA ACAAATATGT TCGATCCAGT ATCCTCTAAC |
| | TATGAACCTA |
| 1001 | CAGTGAACTT CATGACGTGG AAAGAATTGA AGGTTTTACT |
| | AGAGAAAGTA |
| 1051 | AAAGAAAGTC CTATGCACCC AGCGAGTGCT CTTGTTCAGA |
| | AGATATGCGT |
| 1101 | AAATACAACG CACCATCAAA ATCTGTTAAA ACGATGGCAA |
| | TTTGTTCGTA |
| 1151 | ATACGAGTTC ACAATGGACA TCAAGCTTAC CTCAGTATGC |
| | TTTCCACGCC |
| 1201 | CAAACCTACA AACTAGAGAA AAAAATAGAA AGCAGTCTCC |
| | CTATACGATC |
| 1251 | TTCCCTATAA |

The PSORT algorithm predicts inner membrane (0.7198).

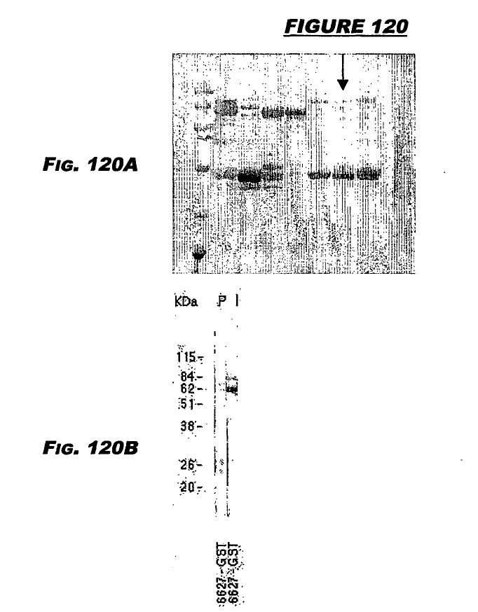

The protein was expressed in E. coli and purified as a GST-fusion product (FIG. 120A). The recombinant protein was used to immunise mice, whose sera were used in a Western blot (FIG. 120B) and for FACS analysis.

These experiments show that cp6627 is a surface-exposed and immunoaccessible protein, and that it is a useful immunogen. These properties are not evident from the sequence alone.

### Example 121

The following C. pneumoniae protein (PID 4376629) was expressed <SEQ ID 241; cp6629>:

| | |
|---|---|
| 1 | MSNITSPVIQ NNRSCNYYFE LKNSTTIHIV ISAILLCGAL |
| | IAFLCVAAPV |
| 51 | SYILSGALLG LGLLIALIGV ILGIKKITPM ISSKEQVFPQ |
| | ELVNRIRAHY |
| 101 | PKFVSDFVSE AKPNLKDLIS FIDLLNQLHS EVGSSTNYNV |
| | SEELQQKIDT |
| 151 | FEGIARLKNE VRTASLKRLE SAASSRPLFP SLPKILQKVF |
| | PFFWLGEFIS |
| 201 | AGSKVVELHR VKKIGGSLEE DLSDYIKPEM LPTYWLIPLD |
| | FRPTNSSILN |
| 251 | LHTLVLARVL TRDVFQHLKY AALNGEWNLN HSDLNTMKQQ |
| | LFAKYHAAYQ |
| 301 | SYKHLSQPSL QEDEFYNLLL CIFKHRYSWK QMSLIKTVPA |
| | DLWENLCCLT |
| 351 | LDHTGRPQDM EFASLIGTLY TQGLIHKESE AFLESLTLLS |
| | LDQFKTIRRQ |
| 401 | STNIAMFLEN IATHNSTFRS LPPITVHPLK RSVFSQPEED |
| | ESSLLIG* |

The cp6629 nucleotide sequence <SEQ ID 242> is:

| | |
|---|---|
| 1 | ATGAGTAATA TAACCTCGCC AGTTATTCAA AATAATCGCT |
| | CTTGTAATTA |
| 51 | TTATTTTGAA TTAAAGAATT CAACCACTAT TCATATTGTT |
| | ATCAGTGCCA |
| 101 | TCTTACTCTG CGGAGCTTTG ATAGCTTTCT TGTGTGTAGC |
| | AGCTCCTGTT |
| 151 | TCCTATATTC TAAGTGGCGC ATTGTTAGGA TTAGGATTAT |
| | TAATAGCCTT |
| 201 | GATTGGTGTG ATTTTAGGAA TAAAAAAAAT CACGCCTATG |
| | ATTTCATCAA |
| 251 | AAGAACAAGT ATTCCCCCAA GAACTCGTAA ATAGAATCAG |
| | GGCGCACTAT |
| 301 | CCTAAATTTG TCTCTGATTT TGTTTCAGAA GCTAAACCAA |
| | ATCTTAAAGA |
| 351 | TCTCATAAGT TTTATTGATC TTCTAAATCA ATTGCACTCT |
| | GAAGTTGGAT |
| 401 | CATCTACAAA TTACAACGTA TCTGAAGAAC TACAACAGAA |
| | AATAGATACG |
| 451 | TTCGAGGGTA TCGCACGCTT AAAAAATGAA GTCCGTACTG |
| | CTTCTCTTAA |
| 501 | AAGACTTGAA AGCGCTGCTT CTTCCCGTCC CCTCTTCCCC |
| | TCTTTACCAA |
| 551 | AAATCTTACA AAAGGTATTT CCATTTTTCT GGTTAGGAGA |
| | GTTTATTTCT |
| 601 | GCAGGCAGCA AGGTTGTAGA GCTCCATCGA GTTAAGAAAA |
| | TTGGAGGCAG |
| 651 | CCTCGAAGAA GACCTTAGTG ATTATATAAA ACCAGAGATG |
| | CTTCCTACCT |
| 701 | ATTGGTTGAT TCCTTTAGAT TTTAGACCAA CAAATTCCTC |
| | TATTCTAAAT |
| 751 | CTACACACAT TAGTTTTAGC TAGAGTCTTA ACTCGTGATG |
| | TTTTTCAACA |
| 801 | TCTTAAGTAT GCAGCATTAA AGTTCGAGTG GAACCTGAAT |
| | CATAGTGATC |
| 851 | TAAATACTAT GAAACAGCAG CTCTTTGCTA AATATCATGC |
| | GGCGTATCAA |
| 901 | TCCTATAAAC ATCTATCTCA ACCCTCTCTT CAAGAGGATG |
| | AATTCTATAA |
| 951 | CCTGCTCTTG TGTATTTTTA AGCATAGGTA CTCGTGGAAG |
| | CAGATGTCCT |
| 1001 | TAATAAAAAC AGTCCCGGCT GATTTATGGG AAAACCTCTG |
| | TTGCTTGACT |
| 1051 | TTAGACCATA CAGGACGACC CCAAGACATG GAATTTGCCT |
| | CTCTAATTGG |
| 1101 | TACTCTCTAC ACACAAGGCC TAATTCATAA AGAAAGCGAA |
| | GCATTTCTTT |
| 1151 | CTTCATTGAC ACTCCTTAGT TTAGATCAGT TTAAAACGAT |
| | CCGTCGTCAG |
| 1201 | TCAACCAATA TAGCGATGTT CCTTGAGAAT TTAGCAACTC |
| | ATAATTCCAC |
| 1251 | CTTTAGAAGC TTACCACCTA TAACAGTCCA TCCACTCAAG |
| | AGAAGCGTCT |
| 1301 | TCTCCCAACC TGAAGAAGAC GAGTCCTCCC TGCTGATAGG |
| | TTAG |

The PSORT algorithm predicts inner membrane (0.5776).

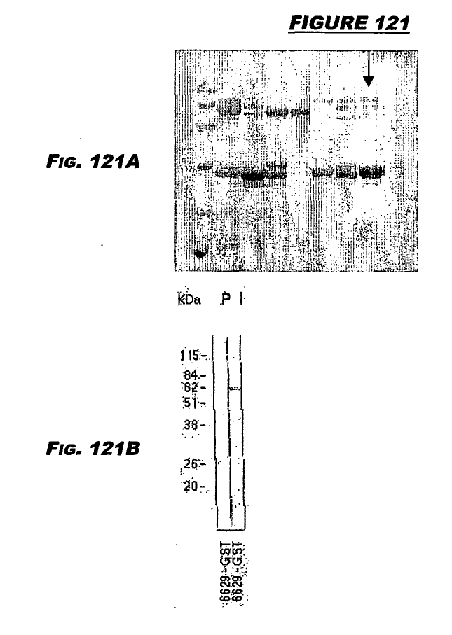

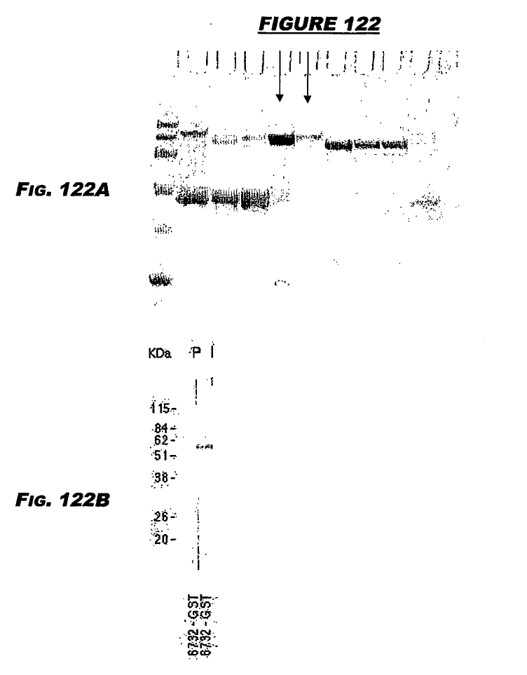

The protein was expressed in E. coli and purified as a GST-fusion product (FIG. 121A). The recombinant protein was used to immunise mice, whose sera were used in a Western blot (FIG. 121B) and for FACS analysis.

These experiments show that cp6629 is a surface-exposed and immunoaccessible protein, and that it is a useful immunogen. These properties are not evident from the sequence alone.

### Example 122

The following C. pneumoniae protein (PID 4376732) was expressed <SEQ ID 243; cp6732>:

| | |
|---|---|
| 1 | MEMMSPFQQP EQCHFDVVGS FLRPESLTRA RSDFEEGRIV |
| | YEQMRVVEDA |
| 51 | AIRNLIKKQT EAGLIFFTDG EFRRYSWDFD FMWGFHGVDR |
| | RRDSNDPEIG |
| 101 | VYLKDKISVS KHPFIEHFEF VKTFEKGNAK AKQTIPSPSQ |
| | FFHEMIFAPN |
| 151 | LKNTRKFYPT NQELIDDIVF YYRQVIQDLY AAGCRNLQLD |
| | DCAWCRLLDI |
| 201 | RAPSWYGVDS HDRLQEILEQ FLWIHNLVMK DRPEDLFVSL |
| | HVCRGDYQAE |
| 251 | FFSRRAYDSI EEPLFAKTDV DSYHYYWALD DKYSGGAEPL |
| | AYVSGEKHVC |
| 301 | LGLISSNHSC IEDRDAVVSR IYEAASYIPL ERLSLSPQCG |
| | FASCEGDHRM |
| 351 | TEEFQWKKIA FVKEIAKEIW G* |

The cp6732 nucleotide sequence <SEQ ID 244> is:

| | |
|---|---|
| 1 | ATGGAAATGA TGAGCCCATT CCAACAACCT GAGCAATGTC |
| | ATTTTGATGT |
| 51 | TGTGGGAAGT TTCTTACGTC CTGAAAGTCT TACACGAGCA |
| | CGCTCTGATT |
| 101 | TTGAAGAAGG AAGAATTGTC TATGAGCAGA TGCGAGTTGT |
| | CGAAGATGCT |
| 151 | GCTATTCGTA ATCTCATAAA AAAGCAAACA GAAGCAGGTC |
| | TTATCTTTTT |
| 201 | TACTGATGGG GAATTCCGTA GGTATAGTTG GGATTTCGAC |
| | TTTATGTGGG |
| 251 | GATTCCATGG CGTGGATCGT CGCAGGGACT CTAATGACCC |
| | TGAAATTGGA |
| 301 | GTGTATCTTA AAGATAAAAT CTCCGTATCA AAACATCCGT |
| | TTATAGAACA |
| 351 | TTTCGAGTTT GTCAAAACTT TTGAGAAGGG AAATGCAAAA |
| | GCAAAACAAA |
| 401 | CGATTCCTTC TCCATCACAA TTTTTCCATG AGATGATTTT |
| | TGCTCCTAAT |
| 451 | CTGAAAAATA CTCGGAAGTT TTATCCTACG AATCAAGAGC |
| | TAATTGATGA |
| 501 | TATTGTCTTT TATTATCGCC AAGTCATCCA AGATCTTTAT |
| | GCTGCAGGTT |
| 551 | GTCGTAATTT GCAGTTGGAC GATTGTGCTT GGTGTCGCCT |
| | CTTGGATATA |
| 601 | CGAGCGCCTT CTTGGTATGG TGTTGATTCT CATGACAGGT |
| | TGCAGGAAAT |
| 651 | TTTAGAACAG TTTTTATGGA TCCATAATTT AGTGATGAAG |
| | GATAGACCCG |
| 701 | AGGATCTTTT TGTAAGTCTG CATGTCTGTC GTGGTGATTA |
| | TCAGGCCGAG |
| 751 | TTTTTCTCTA GACGAGCTTA TGATTCTATA GAGGAGCCTT |
| | TATTTGCTAA |
| 801 | GACCGATGTG GATAGTTATC ACTATTATTG GGCTCTTGAT |
| | GATAAGTATT |
| 851 | CAGGAGGTGC TGAGCCTTTA GCTTACGTCT CTGGAGAGAA |
| | ACACGTCTGC |
| 901 | TTGGGATTGA TCTCCAGCAA CCATTCTTGT ATTGAAGATC |
| | GAGATGCTGT |
| 951 | GGTTTCTCGT ATTTATGAAG CTGCGAGCTA CATTCCCTTA |
| | GAGAGACTTT |
| 1001 | CTTTGAGCCC GCAATCTGGG TTTGCTTCTT GTGAGGGAGA |
| | CCATAGAATG |
| 1051 | ACTGAAGAAG AACAGTGGAA GAAGATCGCC TTTGTGAAAG |
| | AGATTGCTAA |
| 1101 | AGAGATCTGG GGATAA |

The PSORT algorithm predicts cytoplasm (0.2196).

The protein was expressed in E. coli and purified as a GST-fusion product (FIG. 122A). The recombinant protein was used to immunise mice, whose sera were used in a Western blot (FIG. 122B) and for FACS analysis.

These experiments show that cp6732 is a surface-exposed and immunoaccessible protein, and that it is a useful immunogen. These properties are not evident from the sequence alone.

### Example 123

The following C. pneumoniae protein (PID 4376738) was expressed <SEQ ID 245; cp6738>:

| | |
|---|---|
| 1 | VWLRFLLLVS YDEKEKDVVV VCNHSEPNIL GLPPEAVSQL |
| | IEELSDEGYS |
| 51 | YLNVVRCDLS GETTVQQRLL LDANEGRSMT VVISELPEGH |
| | PDIRNLQLAS |
| 101 | ERIFVSREKE AADAYASGCK VVAFDDEHLP WVSSHIAYAE |
| | EIREKQEQTM |
| 151 | QGSLTEEQLG ALLCNTVSTE KNLAFALDAV IKQSVWRFRN |
| | PDLFAYEREA |
| 201 | LEASVTDALV SYVSNLDMIP YTSSQGIVIE DSSIVRTSQE |
| | HTLIVNCAAF |
| 251 | DKLASQIEFL CPSDVLPISG KDPLISDDED EELNPKVSSA |
| | ADSKDKT* |

The cp6738 nucleotide sequence <SEQ ID 246> is:

| | |
|---|---|
| 1 | GTGTGGCTGC GCTTTTTACT TTTAGTGTCC TATGATGAGA |
| | AGGAGAAAGA |
| 51 | CGTAGTTGTC GTTTGTAATC ATTCTGAACC TAATATCCTC |
| | GGCCTGCCTC |
| 101 | CTGAAGCAGT CTCTCAGCTT ATTGAAGAGC TTAGCGATGA |
| | AGGCTATAGC |
| 151 | TATCTGAATG TAGTGCGTTG TGATCTCTCC GGGGAGACTA |
| | CGGTTCAACA |
| 201 | ACGTCTGCTA TTGAATGCCG ATGAAGGGAG ATCTATGACG |
| | GTGGTGATCT |
| 251 | CAGAGCTTCC TGAAGGGCAC CCCGATATTC GGAATTTGCA |
| | GTTGGCATCC |
| 301 | GAAAGAATTT TTGTTTCTCG TGAAAAAGAA GCTGCTGATG |
| | CCTATGCTTC |
| 351 | AGGATGTAAA GTGGTCGCTT TCGATGATGA GCATCTCCCT |
| | TGGGTCTCCA |
| 401 | GTCATATTGC CTACGCGGAG GAGATCAGAG AGAAACAAGA |
| | ACAAACAATG |
| 451 | CAAGGGTCTT TAACTGAAGA GCAGTTAGGA GCACTCCTCT |
| | GCAACACAGT |
| 501 | CTCCACAGAG AAAAATCTAG CCTTTGCTCT AGACGCCGTG |
| | ATAAAACAGT |
| 551 | CTGTGTGGAG ATTCCGCAAT CCGGATCTTT TTGCTTATGA |
| | GAGAGAAGCT |
| 601 | CTAGAGGCTT CATGAACAGA TGCTTTAGTA TCTTACGTTT |
| | CAAATTTAGA |
| 651 | CATGATACCG TACACAAGTT CTCAGGGCAT AGTCATAGAA |
| | GATAGTAGTA |
| 701 | TCGTCCGTAC CTCTCAAGAG CATACACTCA TTGTGAACTG |
| | TGCAGCATTC |
| 751 | GATAAGTTAG CGAGCCAAAT AGAGTTCTTA TGCCCCAGTG |
| | ACGTGTTGCC |
| 801 | CATTTCTGGT AAAGACCCTT TGATTTCTGA TGATGAGGAT |
| | GAGGAACTGA |
| 851 | ATCCTAAAGT TTCATCTGCT GCAGACTCTA AAGATAAAAC |
| | CTAG |

The PSORT algorithm predicts cytoplasm (0.1587).

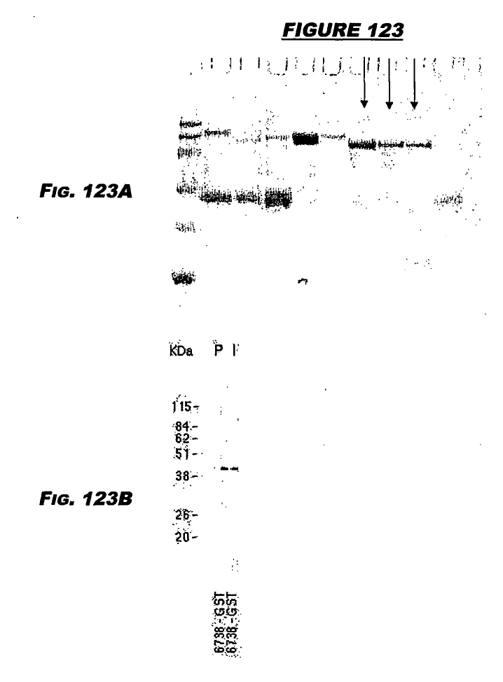

The protein was expressed in E. coli and purified as a GST-fusion product (FIG. 123A). The recombinant protein was used to immunise mice, whose sera were used in a Western blot (FIG. 123B) and for FACS analysis.

These experiments show that cp6738 is a surface-exposed and immunoaccessible protein, and that it is a useful immunogen. These properties are not evident from the sequence alone.

### Example 124

The following C. pneumoniae protein (PID 4376739) was expressed <SEQ ID 247; cp6739>:

| | |
|---|---|
| 1 | MTHCLHGWFS VVRHHFVQAF NFSRPLYSRI THFALGVIKA |
| | IPIVGHLVMG |
| 51 | VDWLISHCFE RGVSHPGFPS DIAPILKVEK IAGRDHISRI |
| | ENQLKSLRKT |
| 101 | IEVEDLDKVH GQYQENPYAD MASSEVLKLD KGVHVSELGK |
| | AFSRVRNRIT |
| 151 | RSYSYAPTPQ LDSIAIVGID LVSPEEQENL VRLANEVIQL |
| | YPKSKTTLYL |
| 201 | LIDFNKEWVG DISSDKEKQL RSLGLHSEVQ CLSVLEPQGA |
| | EGEDTKHFDL |
| 251 | MVGCYGKDSY LREGKILQQA LGTSLGTVPW VNVMHTLPSR |
| | YRSRLSLPIN |
| 301 | IEKDKTELYK EISRTHHQLH TLGMGLGAQD SGLLLDRQRL |
| | HAPLSQGSHC |
| 351 | HSYLADLTHE ELKILLFSAF VDAKNISKKE LREVSLNFAN |
| | DTDVECECAF |
| 401 | YF* |

The cp6739 nucleotide sequence <SEQ ID 248> is:

| | |
|---|---|
| 1 | ATGACTCATT GCTTACATGG TTGGTTTTCT GTAGTTCGTC |
| | ATCACTTTGT |
| 51 | GCAGGCGTTT AGTTTCTCAC GTCCTTTATA TTCTCGAATT |
| | ACCCACTTCG |
| 101 | CTTTAGGGGT GATTAAGGCC ATCCCCATTG TAGGGCATCT |
| | TGTTATGGGA |
| 151 | GTCGATTGGT TGATCTCTCA TTGCTTCGAG AGGGGAGTCT |
| | CACACCCTGG |
| 201 | GTTCCCTTCA GATATTGCTC CTATACTGAA AGTAGAAAAG |
| | ATCGCGGGCC |
| 251 | GAGATCATAT TTCTAGAATC GAAAATCAGC TAAAGAGCCT |
| | TAGGAAAACT |
| 301 | ATCCAGGTTG AAGATCTAGA TAAAGTCCAC GGGCAATATC |
| | AAGAGAATCC |
| 351 | TTATGCAGAT ATGGCCTCTA GTGAGGTTCT TAAACTCGAT |
| | AAGGGAGTTC |
| 401 | ATGTTAGCGA GCTTGGCAAA GCCTTTTCTA GAGTTCGCAA |
| | TCGCATCACC |
| 451 | AGATCCTATA GTTATGCCCC TACTCCTCAG TTGGACTCTA |
| | TAGCTATTGT |
| 501 | TGGTATAGAT CTCGTCAGTC CTGAAGAACA AGAGAATTTA |
| | GTACGCTTGG |
| 551 | CGAATGAGGT CATTCAACTC TATCCCAAAT CAAAGACAAC |
| | TCTATATCTT |
| 601 | CTTATCGATT TTAATAAGGA GTGGGTAGGG GATATCTCCT |
| | CTGATAAGGA |
| 651 | AAAACAGCTC CGTTCTCTAG GTCTACATTC TGAAGTTCAG |
| | TGTCTTTCCG |
| 701 | TCTTGGAACC TCAGGGTGCC GAGGGCGAAG ATACGAAACA |
| | CTTTGACCTT |
| 751 | ATGGTCGGCT GTTATGGGAA GGATTCTTAC TTAAGGGAGG |
| | GTAAAATTTT |
| 801 | ACAGCAGGCC CTAGGGACTT CGTTAGGTAC TGTTCCCTGG |
| | GTGAATGTTA |
| 851 | TGCACACATT GCCATCTAGG TATAGATCTC GGCTTTCCTT |
| | ACCTATAAAT |
| 901 | ACCGAAAAGG ATAAGACAGA GCTTTATAAA GAGATTTCTC |
| | GTACACACCA |
| 951 | TCAGTTGCAT ACTTTGGGAA TGGGACTTGG AGCCCAGGAT |
| | TCAGGATTGC |
| 1001 | TCTTAGACCG GCAACGACTC CATGCTCCTT TATCTCAAGG |
| | GTCTCACTGC |
| 1051 | CATTCCTATC TTGCAGATCT CACCCATGAA GAGCTGAAAA |
| | TTTTGTTATT |
| 1101 | TTCAGCATTT GTGGATGCTA AGAACATAAG TAAGAAAGAG |
| | CTTCGTGAGG |
| 1151 | TATCTCTAAA TTTTGCTAAC GATACTTCCG TAGAGTGTGG |
| | CTGCGCTTTT |
| 1201 | TACTTTTAG |

The PSORT algorithm predicts inner membrane (0.2190).

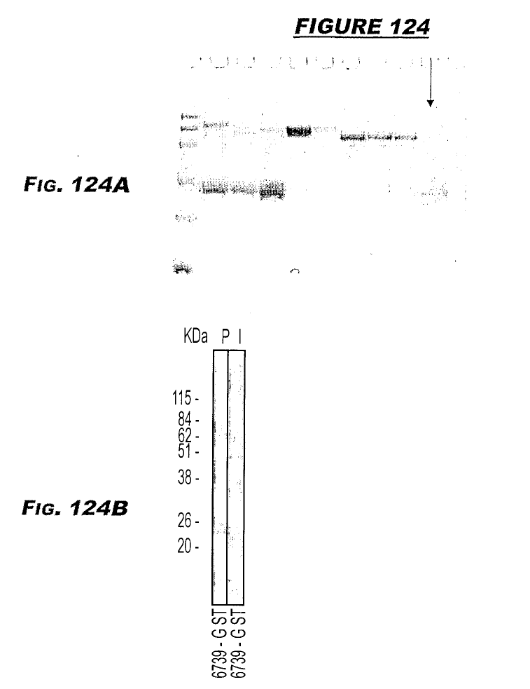

The protein was expressed in E. coli and purified as a GST-fusion product (FIG. 124A). The recombinant protein was used to immunise mice, whose sera were used in a Western blot (FIG. 124B) and for FACS analysis.

These experiments show that cp6739 is a surface-exposed and immunoaccessible protein, and that it is a useful immunogen. These properties are not evident from the sequence alone.

### Example 125

The following C. pneumoniae protein (PID 4376741) was expressed <SEQ ID 249; cp6741>:

| | |
|---|---|
| 1 | MASCLSAWFS IVREHFYRAF DFSLPFCARI TEFVLGVIKG |
| | IPVVGHIIVG |
| 51 | IEWLVSRYLE SFVTKPTFVS DVVSLLKTEK VAGRDHIARV |
| | VETLKRQRVA |
| 101 | VAPEDEDKVH GKIPVHPFGG IQPVEVLTLY PEVQDATLGL |
| | AFSKIRNRVR |
| 151 | QAYLQAPRPK LQKIYIIGND MNPFEVDDFL HLARLCNETQ |
| | RLYPDATISL |
| 201 | YLTASGGRNA MDKKNRKLLS DCELNPKIAC LDFNQGDVVK |
| | QATCDCWMVY |
| 251 | HGENDQGTLN QIQEELEKSG EETPWIHVGQ KPLSQSLWDF |
| | SPFSSLEMKG |
| 301 | DKEKALEYSE LEKEQLYSRL VYVGERSSVL SLGFGDSRSG |
| | ILMDPKRVHA |
| 351 | PLSEGHYCHS YLADIENPGL QKTILAAFLN PKELSSTILQ |
| | PISLNLILNS |
| 401 | KTYLRQHFGF FERMSRSDRN VVVVVCDSWW GTDWKEEPSF |
| | QHFIMELECR |
| 451 | GYSHFNIFAF RSNSMCVEER RILNESSQEK AFTMIFCEDS |
| | VSQGDIRCLH |
| 501 | LASEGMLCGK ECYAVDVYTS GCANFMMEEV LTLERESNLW |
| | NRKHGLWKRE |
| 551 | VRKQKQEAAL DQDESEIYVC NQLTAQQNFA GS* |

The cp6741 nucleotide sequence <SEQ ID 250> is:

| | |
|---|---|
| 1 | ATGGCTTCTT GTTTATCTGC CTGGTTTTCT ATAGTTCGTG |
| | AGCACTTTTA |
| 51 | TCGAGCCTTT GATTTTTCTT TGCCGTTTTG TGCTCGTATT |
| | ACGGAATTTG |
| 101 | TATTAGGGGT CATCAAGGGG ATCCCTGTTG TGGGTCACAT |
| | TATTGTTGGG |
| 151 | ATAGAGTGGC TCGTTTCTAG GTATTTAGAG AGTTTCGTGA |
| | CCAAGCCGAC |
| 201 | ATTTGTCTCT GATGTGGTGA GTCTTCTGAA AACAGAGAAA |
| | GTTGCTGGTC |
| 251 | GCGATCACAT TGCTCGTGTA GTGGAGACTT TGAAGAGGCA |
| | GAGAGTCGCT |
| 301 | GTGGCTCCTG AAGATGAGGA TAAGGTCCAT GGGAAGATTC |
| | CTGTGCATCC |
| 351 | TTTCGGGGGA ATCCAACCTG TAGAAGTTCT CACTCTCTAT |
| | CCCGAAGTTC |
| 401 | AAGATGCAAC GTTAGGGCTT GCCTTCTCTA AAATTCGTAA |
| | TCGTGTAAGA |
| 451 | CAGGCGTATT TGCAAGCTCC ACGGCCAAAA CTGCAGAAGA |
| | TTTACATCAT |
| 501 | AGGAAACGAT ATGAATCCTT TTGAAGTTGA CGACTTCTTG |
| | CATCTAGCCC |
| 551 | GTCTCTGTAA TGAAACTCAA AGACTCTATC CTGACGCTAC |
| | GATTTCTCTA |
| 601 | TATCTAACAG CTTCTGGTGG TCGCAATGCT ATGGACAAAA |
| | AGAATCGGAA |
| 651 | GTTACTTAGT GATTGCGAAC TAAACCCCAA GATTGCTTGT |
| | TTGGACTTTA |
| 701 | ATCAGGGTGA TGTAGTCAAA CAAGCAACTT GTGACTGTTG |
| | GATGGTGTAT |
| 751 | CATGGGGAGA ATGATCAAGG TACGTTGAAT CAGATTCAGG |
| | AAGAGTTAGA |
| 801 | AAAGTCAGGG GAGGAAACCC CTTGGATTCA TGTGGGGCAA |
| | AAGCCTCTTT |
| 851 | CACAATCCTT GTGGGATTTC TCTCCATTTT CATCTTTGGA |
| | GATGAAGGGA |
| 901 | CATAAAGAGA AAGCTCTAGA GTACTCTGAA TTAGAAAAAG |
| | AACAGCTATA |
| 951 | TTCTCGATTG GTATACGTAG GAGAGCGCTC TTCGGTTCTT |
| | AGTTTGGGGT |
| 1001 | TTGGAGATAG TCGGTCAGGG ATCTTGATGG ACCCAAAACG |
| | GGTGCATGCT |
| 1051 | CCCTTATCTG AAGGGCATTA TTGTCATTCC TACCTTGCAG |
| | ACTTAGAAAA |
| 1101 | TCCCGGGTTA CAAAAAACAA TTTTAGCGGC ATTTCTGAAT |
| | CCTAAGGAGT |
| 1151 | TGAGCAGTAC CATACTGCAA CCTATATCTC TAAATCTTAT |
| | CTTAAATAGC |
| 1201 | AAAACTTACT TAAGGCAGCA CTTTGGCTTT TTTGAGAGGA |
| | TGAGCAGAAG |
| 1251 | TGATCGCAAT GTGGTTGTCG TTGTATGTGA TTCTTGGTGG |
| | GGTACCGACT |
| 1301 | GGAAGGAGGA GCCAAGCTTC CAACACTTTA TTATGGAGCT |
| | AGAGTGTCGA |
| 1351 | GGGTATTCGC ACTTCAATAT TTTTGCCTTT AGATCTAATA |
| | GCATGTGTGT |
| 1401 | AGAAGAACGT AGGATCTTAA ATGAAAGTTC TCAAGAGAAA |
| | GCCTTTACCA |
| 1451 | TGATTTTCTG TGAGGATTCA GTATCTCAAG GAGATATCCG |
| | CTGTTTGCAT |
| 1501 | TTGGCGTCTG AAGGAATGCT TTGTGGTAAA GAGTGCTATG |
| | CTGTCGATGT |
| 1551 | CTATACGTCA GGATGCGCGA ACTTTATGAT GGAAGAAGTC |
| | TTAACTTTGG |
| 1601 | AGCGAGAATC TAATCTGTGG AATAGAAAGC ATGGTCTTTG |
| | GAAAAGAGAA |
| 1651 | GTTAGAAAAC AGAAACAAGA AGCTGCTTTG GATCAAGACG |
| | AGAGCGAGAT |
| 1701 | TTACGTTTGT AATCAGCTGA CGGCGCAACA GAACTTCGCT |
| | TGTTCTTGA |

The PSORT algorithm predicts inner membrane (0.2869).

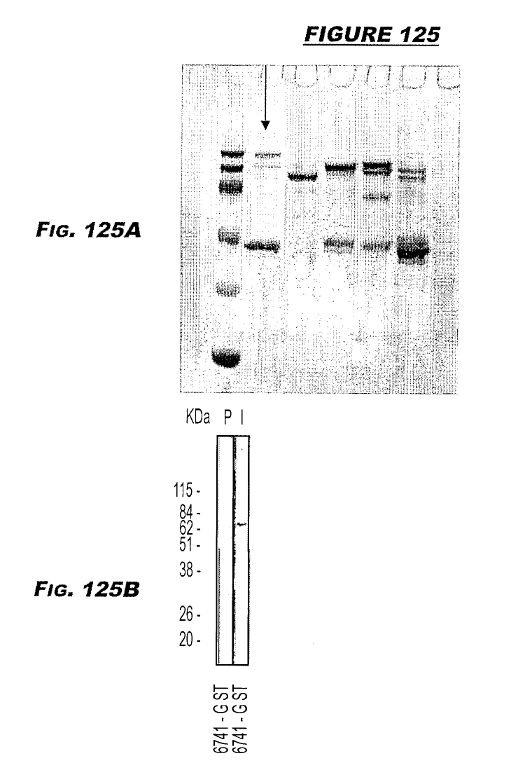

The protein was expressed in E. coli and purified as a GST-fusion product (FIG. 125A). The recombinant protein was used to immunise mice, whose sera were used in a Western blot (FIG. 125B) and for FACS analysis.

These experiments show that cp6741 is a surface-exposed and immunoaccessible protein, and that it is a useful immunogen. These properties are not evident from the sequence alone.

### Example 126

The following C. pneumoniae protein (PID 4376742) was expressed <SEQ ID 251; cp6742>:

| | |
|---|---|
| 1 | LFVSNFIFFV VMPIPYISSW ISTVRQHFVK AFDFSRPFCS |
| | RVTNFALGVI |
| 51 | KAIPIVGHIV MGMEWLVSSC VAGIITRSSF TSDVVQIVKT |
| | EKALGRDHIS |
| 101 | RVAFILQRER GTITPENQDK VHGKFPVCPF GRLKSEETLK |
| | LKPGEREGTL |
| 151 | DTVFSPIRTR VTRAYLQAPR PEIRTISIVG SKLKTPQDFS |
| | QFVSLANETQ |
| 201 | RLHPEALVCL YLTGLNRESQ MCDTTTAEKK QYLHNSGLDS |
| | RIQCKDSKFD |
| 251 | DAGSPENPEL WIGYYSREQQ HNIDGQYIQQ CLGKSADPIP |
| | WIHVTEDTKD |
| 301 | FYYPPNFTSY SHTRQSTDPT SPPRLPESEG DKDSLYGQLS |
| | RSYHHEYMLG |
| 351 | LGLKPEDAGL LMDPDRIYAP LSQGHYCHSY LADIENEDLR |
| | TLVLSPFLDP |
| 401 | GNLSSEDLRP VAFNIARLPL ELDSLFFRLV AGQQEGRNIV |
| | TLAHGTPRPE |
| 451 | DLDPDSMNIL TRRLQMSGYS YLNIFSYKSR KMIVKERQFF |
| | GDRSEGKSFT |
| 501 | LILFEDPISA ADFRCLQLAA EGMVAKDLPS VADICASGCS |
| | CIQFSEMQSP |
| 551 | QAIEYRQWEA RVEDEAGEEA REPVIYSQDQ LSSMLTTQQN |
| | FVFSLDAVVK |
| 601 | QAIWRFRSKG ILTMERKALG EEFLTAIFSY LGSQERNENM |
| | GKRRIEEHFV |
| 651 | VISFEELDRM VQVLPAEVPA DSGNDPTRPV PNPDSNPDSS |
| | QNEGS* |

The cp6742 nucleotide sequence <SEQ ID 252> is:

| | |
|---|---|
| 1 | TTGTTTGTTT CTAATTTTAT TTTTTTTGTT GTTATGCCAA |
| | TTCCCTATAT |
| 51 | TTCTTCTTGG ATTTCTACCG TTCGACAGCA TTTTGTTAAG |
| | GCGTTTGATT |
| 101 | TCTCTCGTCC CTTTTGTTCT AGGGTTACGA ATTTTGCTTT |
| | AGGGGTCATC |
| 151 | AAGGCCATCC CTATTCTAGG ACATATTGTC ATGGGGATGG |
| | AGTGGTTAGT |
| 201 | TTCTTCCTGT GTTGCCGGGA TTATTACTAG GTCCTCCTTT |
| | ACCTCAGATG |
| 251 | TCGTTCAGAT TGTAAAGACT GAGAAGGCGT TAGGTCGAGA |
| | TCATATATCT |
| 301 | CGAGTGGCGG AGATATTGCA AAGAGAAAGG GGGACCATAA |
| | CTCCTGAGAA |
| 351 | TCAAGATAAG GTGCATGGGA AGTTTCCTGT CTGTCCTTTT |
| | GGTCGTTTAA |
| 401 | AATCCGAGGA AACTTTAAAA CTTAAGCCGG GAGAAAGAGA |
| | GGGAACTTTA |
| 451 | GATACTGTAT TTTCTCCGAT TCGCACGCGC GTGACTCGTG |
| | CGTACTTACA |
| 501 | GGCCCCCCGA CCCGAAATAC GTACGATTTC TATTGTGGGT |
| | TCGAAACTTA |
| 551 | AAACTCCTCA AGATTTCTCG CAATTTGTGA GTCTCGCGAA |
| | TGAAACGCAG |
| 601 | AGACTGCATC CTGAAGCGTT AGTTTGTCTG TATTTGACAG |
| | GCTTGAATCG |
| 651 | CGAATCTCAG ATGTGCGATA CAACTACTGC AGAGAAGAAG |
| | CAGTACCTAC |
| 701 | ATAACTCAGG TCTCGACTCT AGAATCCAGT GCAAAGACAG |
| | TAAAGAAGAC |
| 751 | GACGCTGGCT CTCCTGAAAA TCCCGAACTT TGGATTGGCT |
| | ATTATTCACG |
| 801 | AGAGCAACAG CATAATATAG ACGGGCAGTA TATTCAGCAG |
| | TGTCTAGGGA |
| 851 | AGAGTGCAGA TCCAATTCCT TGGATTCATG TTACTGAAGA |
| | CACAAAGGAT |
| 901 | TTTTATTACC CACCAAACTT TACTTCATAC TCACATACAA |
| | GACAATCTAC |
| 951 | AGACCCAACA TCGCCACCAA GACTCCCTGA AAGTGAGGGG |
| | GATAAGGATT |
| 1001 | CCTTGTACGG ACAACTGAGT CGATCGTATC ACCATGAGTA |
| | TATGCTTGGT |
| 1051 | TTGGGATTAA AACCAGAGGA TGCAGGACTC CTGATGGACC |
| | CGGATAGAAT |
| 1101 | CTATGCTCCT CTATCCCAAG GGCATTATTG TCATTCCTAC |
| | CTTGCGGATA |
| 1151 | TAGAAAATGA GGATCTACGA ACTTTAGTCC TTTCGCCTTT |
| | CCTAGATCCT |
| 1201 | GGCAATCTTA GTAGCGAGGA TCTTCGTCCT GTAGCATTCA |
| | ATATCGCTAG |
| 1251 | ATTGCCATTA GAATTGGACT CGTTATTTTT CCGCCTTGTT |
| | GCGGGTCAGC |
| 1301 | AAGAAGGGAG AAACATAGTT ACCCTTGCCC ACGGAACTCC |
| | TCGTCCAGAA |
| 1351 | GATCTTGATC CTGACTCAAT GAACATTCTG ACCAGAAGAT |
| | TACAAATGTC |
| 1401 | TGGATATAGC TATTTGAACA TTTTCTCCTA TAAATCACGG |
| | AAAATGATTG |
| 1451 | TAAAAGAACG TCAGTTCTTT GGAGATCGTT CTGAAGGGAA |
| | GTCTTTCACA |
| 1501 | TTGATCTTAT TTGAGGATCC CATTAGTGCA GCAGATTTCC |
| | GTTGTTTGCA |
| 1551 | GCTAGCTGCA GAAGGTATGG TTGCTAAGGA TCTCCCCAGC |
| | GTAGCAGATA |
| 1601 | TTTGTGCCTC TGGATGTTCC TGCATTCAGT TTTCTGAGAT |
| | GCAGAGTCCT |
| 1651 | CAGGCTATTG AATATAGACA ATGGGAGGCA CGTGTCGAAG |
| | ATGAAGCAGG |
| 1701 | AGAAGAAGCC AGAGAACCAG TAATTTATTC TCAGGATCAA |
| | TTGAGCAGCA |
| 1751 | TGCTCACTAC ACAACAGAAT TTTGTATTTT CTCTAGATGC |
| | TGTGGTAAAA |
| 1801 | CAGGCGATCT GGAGATTCCG TTCGAAAGGT CTTCTTACTA |
| | TGGAAAGAAA |
| 1851 | GGCACTAGGC GAGGAGTTCT TAACTGCGAT ATTTTCCTAT |
| | TTAGGGAGTC |
| 1901 | AGGAGCGTAA TGAGAATATG GGGAAAAGAA CTACCGAAGA |
| | ACATGAGGTC |
| 1951 | GTTATCAGCT TCGAAGAGCT AGATCGCATG GTGCAAGTCC |
| | TCCCAGCCGA |
| 2001 | AGTCCCTGCA GATTCAGGCA ATGATCCTAC GCGTCCCGTT |
| | CCTAATCCAG |
| 2051 | ATAGTAACCC TGATTCCTCG CAAAATGAAG GCAGTTAG |

The PSORT algorithm predicts inner membrane (0.2338).

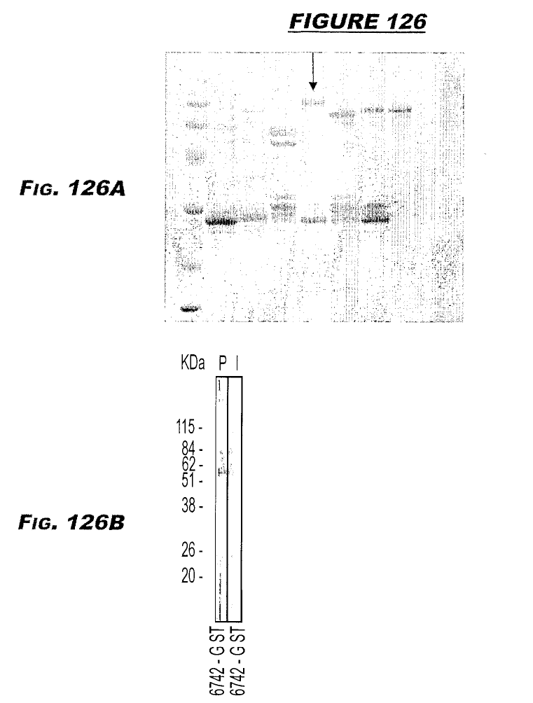

The protein was expressed in E. coli and purified as a GST-fusion product (FIG. 126A). The recombinant protein was used to immunise mice, whose sera were used in a Western blot (FIG. 126B) and for FACS analysis.

These experiments show that cp6742 is a surface-exposed and immunoaccessible protein, and that it is a useful immunogen. These properties are not evident from the sequence alone.

### Example 127

The following C. pneumoniae protein (PID 4376744) was expressed <SEQ ID 253; cp6744>:

| | |
|---|---|
| 1 | VIQHLLNFAL EETPSISVQY QEQEKLSPCD HSPEIGKKKR |
| | WNKLESFSTY |
| 51 | CSLFMSVKDH YKLNLGIQNS LSGWLLDPYR VCAPLSSPYS |
| | CPSYLLDLQN |
| 101 | KELRRSLLST FLDPKNLTSE TFRSVSINFG NSSFGQRWSE |
| | FLSRVLHDEK |
| 151 | EKHVAVVCND AKLLEEGLSP EALSLLEEDL RESGYSYLNI |
| | LSVSPEGVSK |
| 201 | VQERQILRRD LQGRSFTVMI IDLPLGSEDI RSLQLASDRI |
| | LVSSSLDAAD |
| 251 | ACASGCKVLV YENPNASWAQ ELENFYKQVE RRR* |

The cp6744 nucleotide sequence <SEQ ID 254> is:

| | |
|---|---|
| 1 | GTGATACAAC ATCTTCTAAA CTTTGCTCTA GAAGAGACCC |
| | CTTCCATTTC |
| 51 | CGTGCAATAC CAAGAACAAG AGAAGCTCTC TCCGTGCGAT |
| | CATTCCCCAG |
| 101 | AAATAGGTAA AAAGAAAAGA TGGAATAAGC TGGAATCCTT |
| | CTCCACGTAT |
| 151 | TGTTCTCTGT TTATGTCTGT TAAGGATCAT TATAAGCTGA |
| | ATCTAGGAAT |
| 201 | TCAGAATTCC CTGTCAGGGT GGCTTCTGGA TCCCTATAGG |
| | GTTTGCGCGC |
| 251 | CTTTATCTTC ACCGTACTCG TGTCCTTCCT ATCTTTTAGA |
| | TTTGCAAAAC |
| 301 | AAAGAGCTAC GTCGTTCCCT TCTGTCAACG TTTCTAGACC |
| | CTAAAAATCT |
| 351 | CACTAGCGAA ACATTCCGTT CTGTCTCTAT AAACTTTGGC |
| | AACTCTTCGT |
| 401 | TTGGACAGAG ATGGTCAGAG TTTCTATCTC GTGTTCTGCA |
| | CGACGAGAAA |
| 451 | GAAAAGCACG TAGCTGTTGT TTGTAATGAT GCAAAACTTC |
| | TGGAAGAAGG |
| 501 | ATTGTCCCCA GAGGCATTGT CTCTATTAGA AGAAGACTTA |
| | AGAGAATCAG |
| 551 | GGTATTCGTA TCTAAACATT CTCTCGGTGA GCCCCGAAGG |
| | AGTCTCCAAG |
| 601 | GTTCAGGAAC GTCAGATTCT AAGGCGAGAT CTCCAAGGAC |
| | GGTCCTTTAC |
| 651 | TGTCATGATT ACAGATCTTC CTTTAGGTAG CGAAGATATC |
| | CGTAGTTTAC |
| 701 | AATTAGCCTC GGATAGGATT TTAGTCTCCA GTTCTCTTGA |
| | TGCCGCGGAT |
| 751 | GCATGTGCTT CGGGATGTAA AGTCTTAGTC TACGAAAATC |
| | CAAATGCATC |
| 801 | CTGGGCTCAG GAATTGGAGA ACTTCTACAA ACAAGTTGAG |
| | AGAAGAAGGT |
| 851 | AG |

The PSORT algorithm predicts cytoplasm (0.3833).

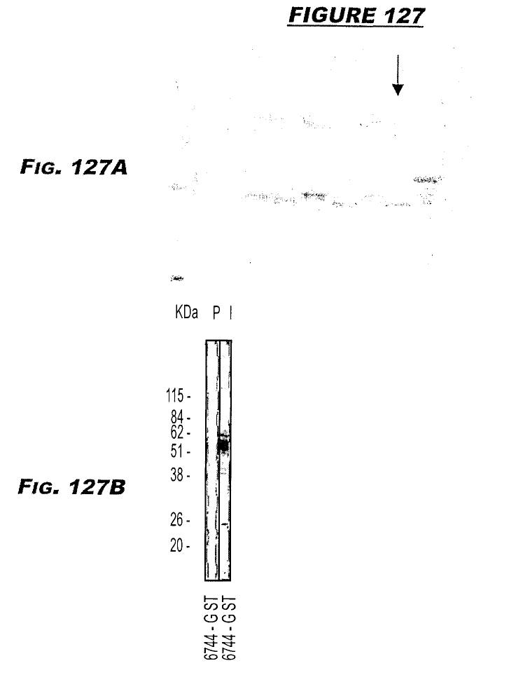

The protein was expressed in E. coli and purified as a GST-fusion product (FIG. 127A). The recombinant protein was used to immunise mice, whose sera were used in a Western blot (FIG. 127B) and for FACS analysis.

These experiments show that cp6744 is a surface-exposed and immunoaccessible protein, and that it is a useful immunogen. These properties are not evident from the sequence alone.

### Example 128

The following C. pneumoniae protein (PID 4376745) was expressed <SEQ ID 255; cp6745>:

| | |
|---|---|
| 1 | VACPSISSWF TVVRQHFVNA FDFTHPVCSR ITNFALGIIK |
| | AIPVLGHIVM |
| 51 | GIEWLISWIP RHTVRHGMFT SDVSSAIEVE QTRGHNCLAP |
| | LEAYLSSLRV |
| 101 | PISQEDLGKV HGRTPEDPFV DITPTEIVQL LPDEELSTVD |
| | EALQGVRSRL |
| 151 | TYAYRSVEKP MIQDLALVGF GLRDSADLIN FVRLANGVQN |
| | HYPHTKVKLY |
| 201 | LAKNLADVWD CEISEEEKGQ LRALGLDPKI ESISLTSAGL |
| | PSVPEVATVD |
| 251 | FMITCYGKDQ EVQDP* |

The cp6745 nucleotide sequence <SEQ ID 256> is:

| | |
|---|---|
| 1 | GTGGCTTGTC CAAGTATTTC TTCTTGGTTT ACTGTCGTTC |
| | GACAGCATTT |
| 51 | TGTAAACGCC TTTGATTTCA CCCATCCCGT TTGTTCTCGG |
| | ATTACAAATT |
| 101 | TTGCTTTGGG GATCATTAAG GCAATTCCCG TATTAGGACA |
| | CATTGTCATG |
| 151 | GGAATCGAGT GGTTGATTTC CTGGATTCCC AGACACACCG |
| | TTCGTCATGG |
| 201 | AATGTTTACT TCTGATGTCT CTAGTGCTAT TAAAGTAGAA |
| | CAAACACGGG |
| 251 | GTCATAATTG TTTAGCTCCC CTAGAAGCCT ATTTAAGTAG |
| | CTTGAGAGTC |
| 301 | CCCATTTCCC AAGAAGATCT AGGCAAAGTA CACGGGAGAA |
| | CCCCAGAAGA |
| 351 | TCCCTTCGTA GATATCACAC CCACAGAAAT TGTCCAACTT |
| | CTCCCTGATG |
| 401 | AAGAACTCTC TACTGTAGAT GAGGCACTGC AAGGCGTTCG |
| | TAGTAGGTTA |
| 451 | ACCTATGCCT ATAGGTCCGT AGAGAAACCT ATGATTCAAG |
| | ATCTTGCTCT |
| 501 | TGTGGGTTTT GGTCTCCGAG ATTCTGCGGA CCTCATAAAT |
| | TTCGTGCGTC |
| 551 | TTGCTAATGG CGTGCAGAAT CACTATCCCC ATACTAAAGT |
| | GAAGCTCTAT |
| 601 | TTAGCGAAGA ACTTGGGAGA TGTCTGGGAC TGTGAAATTT |
| | CTGAAGAGGA |
| 651 | AAAAGGGCAA CTCCGAGCTC TAGGTTTAGA CCCTAAAATA |
| | GAGAGTATAT |
| 701 | CCCTTACGAG TGCAGGTCTT CCTTCAGTGC CAGAAGTCGC |
| | TACTGTCGAT |
| 751 | TTTATGATTA CCTGTTACGG GAAAGATCAG GAAGTCCAAG |
| | ATCCCTAG |

The PSORT algorithm predicts inner membrane (0.2253).

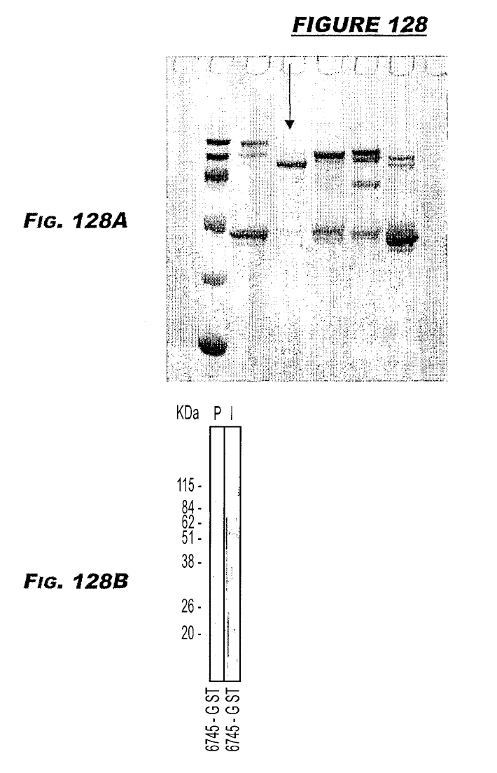

The protein was expressed in E. coli and purified as a GST-fusion product (FIG. 128A). The recombinant protein was used to immunise mice, whose sera were used in a Western blot (FIG. 128B) and for FACS analysis.

These experiments show that cp6745 is a surface-exposed and immunoaccessible protein, and that it is a useful immunogen. These properties are not evident from the sequence alone.

### Example 129

The following C. pneumoniae protein (PID 4376747) was expressed <SEQ ID 257; cp6747>:

| | |
|---|---|
| 1 | MMKQGVGQDA KELYTFLSRG NEHYQPCLWF SLEEELGFLF |
| | DEKMLCAPLS |
| 51 | EDHYCHSYLV DLVDQHLKDL ILSMFLDPQN ISAGELLKVS |
| | INVGDSFSPL |
| 101 | QQKDFLSMVL RDETGKNVVV VFKGVLSLPA TQVCKLVEEL |
| | NSKDYSYLNI |
| 151 | FSCHGDSSPQ LLFRKELEGT SGRYFTVICA LYLGDTDMRS |
| | LQLASERIMV |
| 201 | SREFDLVDAY AARCKLLKID HTNWRPGTFS RHADFADAVD |
| | VSAGFNSREF |
| 251 | KLITQANQGI LESGELPLPS KTFWEGFLAF CDRVTVTRHF |
| | IPMLDAAIKQ |
| 301 | AVWTHKHPSL IDKECEALDL KTQCLPSIVS YLEYVTNSHE |
| | KTSKGPFIQK |
| 351 | EIIADCSPLK EALFPGSDED VPSTSEDPSD DHPSDLEDS* |

The cp6747 nucleotide sequence <SEQ ID 258> is:

| | |
|---|---|
| 1 | ATGATGAAAC AAGGAGTCGG GCAGGATGCT AAAGAGCTAT |
| | ACACATTTCT |
| 51 | ATCTCGTGGG AATGAGCATT ACCAACCGTG TCTATGGTTC |
| | AGTCTCGAAG |
| 101 | AGGAACTCGG ATTCCTTTTC GATGAAAAAA TGCTCTGCGC |
| | CCCTCTATCT |
| 151 | GAGGATCACT ATTGCCACTC GTATCTTGTA GATCTAGTGG |
| | ATCAACATTT |
| 201 | AAAGGATTTA ATATTATCGA TGTTTTTAGA TCCTCAGAAT |
| | ATCTCAGCAG |
| 251 | GAGAACTCCT GAACCTCTCT ATAAACGTTG GAGATTCTTT |
| | TTCTCCTCTA |
| 301 | CAACAGAAAG ATTTCCTCTC GATGGTCTTA GGTGATGAAA |
| | CGGGAAAAAA |
| 351 | CGTCGTCGTG GTTTTTAAAG GAGTTCTCTC CTTACCCGCA |
| | ACCCAAGTCT |
| 401 | GCAAATTAGT AGAGGAATTG AACTCTAAGG ACTACTCCTA |
| | CCTCAATATA |
| 451 | TTTTCTTGTC ACGGAGATAG TAGTCCTCAG CTTTTATTCC |
| | GTAAGGAATT |
| 501 | AGAGGGAACT TCAGGGCGTT ATTTTACAGT GATTTGCGCT |
| | TTATATCTAG |
| 551 | GGGATACAGA CATGCGTAGT TTACAACTTG CTTCTGAAAG |
| | GATCATGGTC |
| 601 | TCTAGAGAGT TTGATCTTGT AGATGCCTAT GCTGCAAGAT |
| | GCAAGCTCTT |
| 651 | GAAAATCGAT CATACAAATT GGAGACCTGG AACTTTCAGT |
| | CGCCACGCCG |
| 701 | ATTTCGCAGA TGCTGTAGAC GTATCAGCAG GATTTAACTC |
| | AAGAGAATTT |
| 751 | AAACTGATTA CGCAGGCGAA TCAAGGGATC CTAGAGTCTG |
| | GAGAACTCCC |
| 801 | GCTCCCTTCA AAAACCTTCT GGGAAGGATT CTTAGCATTC |
| | TGTGATCGAG |
| 851 | TGACTGTCAC GAGACACTTC ATTCCAATGT TAGACGCCGC |
| | TATAAAGCAA |
| 901 | GCGGTATGGA CTCATAAACA TCCCAGCTTG ATAGATAAAG |
| | AGTGTGAAGC |
| 951 | CCTAGACTTG AAAACACAGT GCTTGCCATC TATCGTATCG |
| | TACCTTGAAT |
| 1001 | ATGTCACAAA CTCTCACGAA AAAACATCGA AAGGCCCGTT |
| | CATACAAAAA |
| 1051 | GAGATTATCG CAGACTGTTC TCCTCTTAAA GAGGCGCTCT |
| | TCCCAGGTTC |
| 1101 | TGATGAAGAT GTTCCCTCTA CCTCTGAGGA TCCTTCAGAT |
| | GATCATCCTT |
| 1151 | CGGATCTTGA AGACTCTTAA |

The PSORT algorithm predicts inner membrane (0.1447).

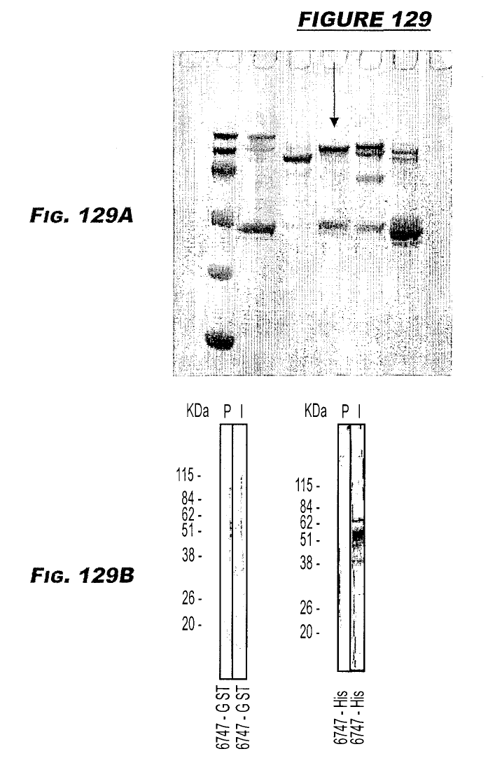

The protein was expressed in E. coli and purified as a GST-fusion product (FIG. 129A) and also as a his-tagged product. The recombinant proteins were used to immunise mice, whose sera were used in a Western blot (FIG. 129B) and for FACS analysis.

These experiments show that cp6747 is a surface-exposed and immunoaccessible protein, and that it is a useful immunogen. These properties are not evident from the sequence alone.

### Example 130

The following C. pneumoniae protein (PID 4376756) was expressed <SEQ ID 259; cp6756>:

| | |
|---|---|
| 1 | MASGIGGSSG LGKIPPKDNG DRSRSPSPKG ELGSHEISLP |
| | PQEHGEEGAS |
| 51 | GSSHIHSSSS FLPEDQESQS SSSAASSPGF FSRVRSGVDR |
| | ALKSFGNFFS |
| 101 | AESTSQARET RQAFVRLSKT ITAGERRDVD SSSAAATEAR |
| | VAEDASVSGE |
| 151 | NPSQGVPETS SGPEPQRLFS LPEVKKQSGL GRLVQTVRDR |
| | IVLPSGAPPT |
| 201 | DSEPLSLYEL NLRLSSLRQE LSDIQSNDQL TPEEKAEATV |
| | TIQQLIQITE |
| 251 | FQCGYMEATQ SSVSLAEARF KGVETSDEIN SLCSELTDPE |
| | LQELMSDGDS |
| 301 | LQNLLDETAD DLEAALSHTR LSFSLDDNPT PIDNNPTLIS |
| | QEEPIYEEIG |
| 351 | GAADPQRTRE NWSTRLWNQI REALVSLLGM ILSILGSILH |
| | RLRIARHAAA |
| 401 | EAVGRCCTCR GEECTSSEED SMSVGSPSEI DETERTGSPH |
| | DVPRRNGSPR |
| 451 | EDSPLMNALV GWAHKHGAKT KESSESSTPE ISISAPIVRG |
| | WSQDSSVSFI |
| 501 | VMEDDHIFYD VPRRKDGIYD VPSSPRWSPA RELEEDVFGD |
| | YEVPITSAEP |
| 551 | SKDKNIYMTP RLAIPAIYDL PSRPGSSGSS RSPSSDRVRS |
| | SSPNRRGVPL |
| 601 | PPVPSPAMSE EGSIYEDMSG ASGAGESDYE DMSRSPSPRG |
| | DLDEPIYANT |
| 651 | PEDNPFTQRN IDRILQERSG GASASPVEPI YDEIRWIHGR |
| | PPATLPRPEN |
| 701 | TLTNVSLRVS PGFGPEVRAA LLSESVSAVM VEAESIVPPT |
| | EPGDGESEYL |
| 751 | EPLGGLVATT KILLQKGWPR GESNA* |

The cp6756 nucleotide sequence <SEQ ID 260> is:

| | |
|---|---|
| 1 | ATGGCATCAG GAATCGGAGG ATCTAGTGGA TTAGGAAAGA |
| | TTCCACCTAA |
| 51 | AGATAATGGG GATAGAAGTC GATCGCCCTC TCCTAAGGGA |
| | GAACTTGGCA |
| 101 | GCCACGAGAT TTCCCTGCCT CCTCAAGAAC ATGGAGAGGA |
| | AGGAGCTTCA |
| 151 | GGATCTTCGC ATATACATAG CAGTTCCTCT TTTCTACCAG |
| | AAGATCAGGA |
| 201 | GTCTCAGAGC TCTTCTTCGG CAGCTTCTAG CCCGGGATTT |
| | TTTTCTCGCG |
| 251 | TACGTTCTGG GGTAGACAGG GCCTTAAAAT CATTTGGCAA |
| | CTTTTTTTCC |
| 301 | GCAGAGTCTA CGAGTCAAGC GCGTGAAACG CGACAAGCTT |
| | TTGTTAGATT |
| 351 | ATCAAAAACC ATCACCGCGG ATGAGAGACG GGATGTCGAT |
| | TCATCAAGTG |
| 401 | CTGCTGCTAC AGAAGCCCGA GTGGCAGAGG ACGCGAGTGT |
| | TTCAGGCGAA |
| 451 | AATCCTTCTC AGGGGGTTCC AGAAACCTCT TCTGGACCAG |
| | AACCTCAGCG |
| 501 | TTTATTTTCT CTTCCTTCAG TAAAAAAACA GAGCGGTTTG |
| | GGTCGGTTGG |
| 551 | TACAGACAGT TCGCGATCGC ATAGTACTTC CTAGTGGGGC |
| | TCCACCTACA |
| 601 | GACAGCGAGC CTTTAAGTCT CTACGAGCTA AACCTCCGTT |
| | TGAGTAGTTT |
| 651 | ACGTCAGGAG CTCTCTGACA TACAAAGTAA TGATCAGTTG |
| | ACTCCAGAGG |
| 701 | AAAAAGCAGA AGCCACAGTT ACCATACAAC AGCTGATCCA |
| | AATTACAGAA |
| 751 | TTCCAATGCG GCTATATGGA GGCAACACAA TCTTCGGTAT |
| | CTCTAGCAGA |
| 801 | AGCTCGTTTT AAGGGGGTAG AAACTAGTGA TGAGATCAAT |
| | TCCCTCTGTT |
| 851 | CAGAACTGAC AGATCCTGAG CTTCAAGAAC TCATGAGTGA |
| | TGGAGACTCT |
| 901 | CTTCAAAACC TATTAGATGA GACTGCCGAC GATTTAGAAG |
| | CTGCTTTGTC |
| 951 | CCATACTCGA TTGAGTTTTT CTTTAGACGA TAATCCAACT |
| | CCGATAGACA |
| 1001 | ATAATCCAAC TCTGATTTCT CAAGAAGAGC CTATTTATGA |
| | GGAAATCGGA |
| 1051 | GGAGCTGCAG ATCCTCAAAG AACTCGGGAA AACTGGTCTA |
| | CAAGATTATG |
| 1101 | GAATCAGATT CGCGAGGCTC TGGTTTCTCT TTTAGGAATG |
| | ATTTTAAGCA |
| 1151 | TTCTAGGGTC CATCTTGCAC AGGTTGCGTA TTGCTCGTCA |
| | TGCAGCTGCT |
| 1201 | GAAGCAGTGG GTCGTTGTTG CACGTGCCGA GGAGAAGAGT |
| | GTACTTCTTC |
| 1251 | TGAAGAGGAC TCGATGTCGG TGGGGTCTCC TTCAGAAATT |
| | GATGAAACTG |
| 1301 | AAAGAACGGG CTCTCCGCAT GACGTTCCAC GCAGAAATGG |
| | AAGTCCACGT |
| 1351 | GAAGATTCTC CATTGATGAA TGCCTTAGTA GGATGGGCAC |
| | ATAAGCACGG |
| 1401 | TGCTAAAACC AAGGAGAGTT CAGAATCAAG TACCCCGGAA |
| | ATTTCGATTT |
| 1451 | CTGCTCCCAT AGTGAGAGGT TGGAGTCAAG ACAGTTCCGT |
| | CAGTTTTATT |
| 1501 | GTTATGGAAG ATGATCATAT TTTCTATGAT GTTCCTCGTA |
| | GAAAAGATGG |
| 1551 | AATCTATGAC GTTCCTAGTT CCCCTAGATG GAGTCCTGCG |
| | CGAGAGTTGG |
| 1601 | AAGAGGATGT TTTTGGAGAT TATGAAGTTC CTATAACCTC |
| | TGCTGAACCA |
| 1651 | TCTAAAGACA AGAACATCTA CATGACACCT AGATTAGCAA |
| | CTCCTGCTAT |
| 1701 | CTATGATCTT CCTTCACGTC CAGGATCGTC TGGAAGCTCA |
| | CGTTCTCCGT |
| 1751 | CTTCAGATCG CGTACGAAGC AGCTCACCAA ATAGACGGGG |
| | TGTGCCTCTT |
| 1801 | CCTCCAGTTC CTTCACCTGC TATGAGTGAG GAGGGGAGCA |
| | TTTATGAGGA |
| 1851 | TATGAGCGGT GCTTCAGGTG CAGGTGAAAG TGATTATGAA |
| | GATATGAGCC |
| 1901 | GTTCCCCCTC TCCTAGAGGC GACTTGGATG AACCCATATA |
| | TGCTAATACT |
| 1951 | CCTGAAGATA ATCCATTTAC TCAGAGAAAT ATAGATAGAA |
| | TTTTACAGGA |
| 2001 | GAGGTCAGGC GGTGCTTCCG CTTCTCCTGT AGAGCCTATT |
| | TATGATGAGA |
| 2051 | TCCCATGGAT TCATGGCAGG CCCCCTGCTA CACTTCCAAG |
| | ACCCGAGAAT |
| 2101 | ACATTGACTA ATGTTTCGCT TAGAGTGAGC CCAGGGTTTG |
| | GACCAGAAGT |
| 2151 | AAGAGCCGCT TTGCTTAGCG AGAGCGTGAG TGCTGTTATG |
| | GTCGAAGCAG |
| 2201 | AGAGTATTGT TCCTCCAACA GAGCCGGGGG ACGGAGAATC |
| | AGAATATCTA |
| 2251 | GAGCCCTTAG GGGGACTTGT AGCTACAACG AAAATCTTAC |
| | TACAAAAAGG |
| 2301 | ATGGCCTCGT GGAGAGTCGA ATGCTTAG |

The PSORT algorithm predicts inner membrane (0.3994).

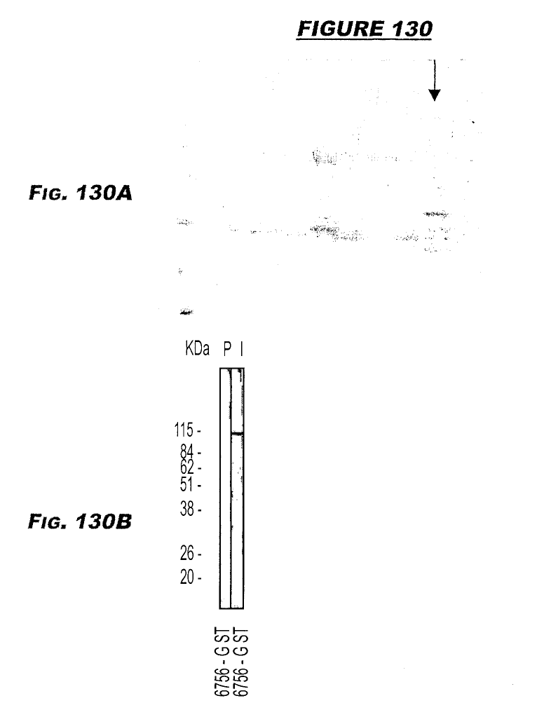

The protein was expressed in E. coli and purified as a GST-fusion product (FIG. 130A). The recombinant protein was used to immunise mice, whose sera were used in a Western blot (FIG. 130B) and for FACS analysis.

These experiments show that cp6756 is a surface-exposed and immunoaccessible protein, and that it is a useful immunogen. These properties are not evident from the sequence alone.

### Example 131

The following C. pneumoniae protein (PID 4376761) was expressed <SEQ ID 261; cp6761>:

| | |
|---|---|
| 1 | MTVAEVKGTF KLVCLGCRVN QYEVQAYRDQ LTILGYQEVL |
| | DSEIPADLCI |
| 51 | INTCAVTASA ESSGRHAVRQ LCRQNPTAHI VVTGCLGESD |
| | KEFFASLDRQ |
| 101 | CTLVSNKEKS RLIEKIFSYD TTFPEFKIHS FEGKSRAFIK |
| | VQDGCNSFCS |
| 151 | YCIIPYLRGR SVSRPAEKIL AEIAGVVDQG YREVVIAGIN |
| | VGDYCDGERS |
| 201 | LASLIEQVDR IPGIERIRIS SIDPDDITED LHRAITSSRH |
| | TCPSSHLVLQ |
| 251 | SGSNSILKRM NRKYSRGDFL DCVEKFRASD PRYAFTTDVI |
| | VGFPGESDQD |
| 301 | FEDTLRIIED VGFIDVHSFP FSARRRTKAY TFDNQIPNQV |
| | IYERKKYLAE |
| 351 | VAKRVGQKEM MKRLGETTEV LVEKVTGQVA TGHSPYFEKV |
| | SFPVVGTVAI |
| 401 | NTLVSVRLDR VEEEGLIGEI V* |

The cp6761 nucleotide sequence <SEQ ID 262> is:

| | |
|---|---|
| 1 | ATGACGGTTG CGGAAGTCAA AGGAACATTT AAGCTGGTCT |
| | GTTTAGGCTG |
| 51 | TCGGGTGAAT CAGTATGAGG TCCAAGCATA TCGCGACCAG |
| | TTGACTATCT |
| 101 | TAGGTTACCA AGAGGTCCTG GATTCTGAAA TCCCTGCAGA |
| | TTTATGCATA |
| 151 | ATCAATACGT GTGCTGTCAC AGCTTCTGCT GAGAGTTCGG |
| | GTCGTCATGC |
| 201 | TGTGCGTCAG TTATGTCGTC AGAACCCTAC AGCACATATT |
| | GTTGTCACAG |
| 251 | GTTGTTTGGG GGAATCTGAC AAAGAGTTTT TTGCTTCTTT |
| | GGATCGGCAA |
| 301 | TGCACACTTG TTTCCAATAA AGAAAAATCC CGACTTATAG |
| | AAAAAATTTT |
| 351 | TTCCTATGAT ACGACCTTCC CTGAGTTCAA GATCCATAGT |
| | TTTGAGGGAA |
| 401 | AGTCTCGAGC TTTTATTAAA GTTCAAGATG GCTGTAATTC |
| | TTTTTGCTCG |
| 451 | TACTGCATTA TTCCTTATTT GCGGGGGCGT TCGGTTTCTC |
| | GTCCTGCTGA |
| 501 | GAAGATTTTA GCTGAAATCG CAGGGGTTGT AGACCAAGGA |
| | TATCGCGAAG |
| 551 | TTGTAATTGC AGGAATTAAT GTTGGAGATT ATTGCGATGG |
| | AGAGCGTTCA |
| 601 | TTAGCCTCTT TGATTGAACA GGTGGACCGG ATTCCTGGAA |
| | TTGAGAGGAT |
| 651 | TCGAATTTCC TCTATAGATC CTGATGATAT CACTGAAGAT |
| | CTGCACCGTG |
| 701 | CCATCACCTC ATCGCGTCAC ACTTGTCCTT CGTCACACCT |
| | TGTTCTTCAA |
| 751 | TCGGGGTCGA ATTCAATTTT AAAGAGAATG AACCGGAAGT |
| | ATTCTCGCGG |
| 801 | AGATTTTTTA GATTGTGTAG AGAAGTTCCG TGCTTCTGAT |
| | CCTCGCTATG |
| 851 | CCTTTACTAC AGATGTGATT GTCGGATTTC CTGGAGAGAG |
| | TGATCAAGAT |
| 901 | TTTGAAGATA CTTTGAGAAT TATTGAAGAT GTAGGCTTTA |
| | TTAAAGTGCA |
| 951 | TAGTTTCCCT TTCAGTGCTC GTCGTCGTAC TAAGGCATAT |
| | ACTTTTGATA |
| 1001 | ATCAGATTCC CAATCAGGTG ATCTATGAGA GGAAGAAGTA |
| | TCTTGCTGAG |
| 1051 | GTTGCTAAGA GGGTAGGCCA GAAAGAGATG ATGAAGCGTT |
| | TAGGAGAGAC |
| 1101 | TACAGAGGTG CTTGTTGAGA AAGTAACGGG GCAGGTTGCT |
| | ACGGGTCACT |
| 1151 | CTCCTTATTT TGAAAAGGTT TCTTTCCCTG TTGTAGGAAC |
| | GGTAGCTATC |
| 1201 | AACACTCTAG TTTCTGTGCG TCTTGATAGG GTAGAGGAAG |
| | AAGGGCTGAT |
| 1251 | TGGGGAGATT GTATGA |

The PSORT algorithm predicts inner membrane (0.1574).

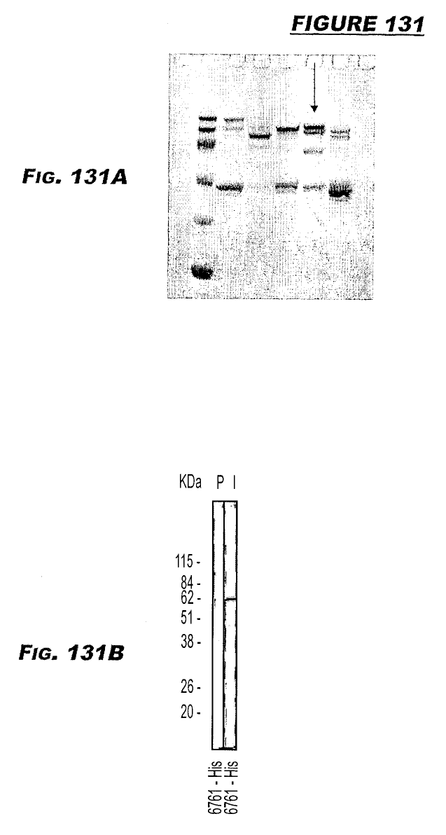

The protein was expressed in E. coli and purified as a GST-fusion product (FIG. 131A) and also as a his-tagged product. The recombinant proteins were used to immunise mice, whose sera were used in a Western blot (FIG. 131B) and for FACS analysis.

These experiments show that cp6761 is a surface-exposed and immunoaccessible protein, and that it is a useful immunogen. These properties are not evident from the sequence alone.

### Example 132

The following C. pneumoniae protein (PID 4376766) was expressed <SEQ ID 263; cp6766>:

| | |
|---|---|
| 1 | MATSVPVTSS TSVGEANSSN ERFTERTSRM YYAALVLGAL |
| | SCLIFIAMIV |
| 51 | IFPQVGLWAV VLGFALGCLL LSLAIVFAVS GLVLGKTLEP |
| | SREATPPEIV |
| 101 | AQKEWTTQQD VLGNEYWRSE LISLFLRGDL HESLIVDSKD |
| | RSLDIDQSLQ |
| 151 | NILKLEPLST TLSLLKKDCV HINIILHLVR QWNLLGVDLS |
| | PEVTAHAEEL |
| 201 | LLFLIEEQYY SPDILKLIRY GDALQATSPL MDWADSGSFS |
| | VDADGVFSCR |
| 251 | REECSPEDAL AQFDLLLALE NPDRRFLKDS FLTYIWSSSF |
| | FEKFLHRHLE |
| 301 | SLQRKLPETA IDVARYFAQI QTFLSRYFQK LDLINAMSLD |
| | WGYNCAEGEK |
| 351 | CYESANQRLD NLFIAFSSSV PAMKRLFDKY GSVVRVDRRQ |
| | IREQILSNTE |
| 401 | ILENESGFLC SLYEYPLSYL IDWAVLLDCV RGTEISLEDQ |
| | ADYTVCLQGL |
| 451 | DSMLSQFASR LQSGQKVLNP RDVLSEQAAV MLVEGLAAQG |
| | VSFQGLKALM |
| 501 | YLIAVPQRMW LGALPLFESF PVFNRMKEFL GESLGD* |

The cp6766 nucleotide sequence <SEQ ID 264> is:

| | |
|---|---|
| 1 | ATGGCAACCT CTGTTCCTGT AACTTCATCT ACTTCTGTAG |
| | GAGAGGCTAA |
| 51 | CTCCTCCAAC GAAAGATTTA CTGAACGAAC ATCGCGAATG |
| | TATTACGCAG |
| 101 | CTTTAGTCCT AGGGGCTTTG AGCTGTTTAA TTTTTATTGC |
| | TATGATTGTC |
| 151 | ATTTTCCCAC AGGTCGGATT GTGGGCTGTG GTCCTCGGGT |
| | TTGCTCTTGG |
| 201 | ATGTTTACTT TTAAGCTTAG CTATCGTTTT TGCTGTCTCC |
| | GGTCTCGTTT |
| 251 | TAGGCAAGAC TTTAGAACCT AGTCGAGAAG CGACTCCTCC |
| | AGAAATTGTT |
| 301 | GCGCAAAAGG AGTGGACTAC ACAACAAGAT GTCTTAGGGA |
| | ATGAGTATTG |
| 351 | GCGTTCCGAG TTGATTTCCT TGTTCTTACG AGGGGATCTC |
| | CACGAATCTC |
| 401 | TGATTGTTGA TTCTAAGGAT CGATCTTTAG ATATTGATCA |
| | GAGTTTACAA |
| 451 | AATATATTGA AACTTGAGCC CCTATCTACG ACACTTTCGC |
| | TGTTAAAGAA |
| 501 | AGATTGTGTC CACATCAATA TCATTTTACA TTTAGTGAGA |
| | CAGTGGAACT |
| 551 | TACTGGGAGT GGATCTTAGT CCTGAAGTCA CTGCGCACGC |
| | CGAGGAACTT |
| 601 | CTACTCTTTT TGATAGAAGA GCAGTATTAC TCTCCTGATA |
| | TTTTGAAATT |
| 651 | GATTCGCTAC GGAGATGCTT TACAAGCAAC GTCTCCTTTG |
| | ATGGATTGGG |
| 701 | CAGATTCAGG TTCCTTTAGT GTAGACGCAG ACGGGGTATT |
| | TAGCTGTCGC |
| 751 | AGAGAAGAAT GTTCTCCTGA GGATGCTTTG GCGCAATTCG |
| | ATCTTCTTTT |
| 801 | GGCGTTGGAA AATCCCGACA GACGCTTCTT AAAGGATTCT |
| | TTTCTTACCT |
| 851 | ACATTTGGTC GTCTTCATTT TTTGAGAAGT TTTTACATCG |
| | CCATCTAGAG |
| 901 | AGCTTGCAAA GAAAGCTCCC AGAGACAGCG ATCGATGTCG |
| | CCCGCTATGA |
| 951 | AGCACAAATA CAAACATTTC TCTCTCGCTA TTTTCAGAAG |
| | CTCGATTTGA |
| 1001 | TAAACGCAAT GTCCTTAGAT TGGGGATATA ACTGTGCTGA |
| | GGGAGAAAAA |
| 1051 | TGTTATGAGA GCGCAAATCA AAGATTAGAC AACCTATTTA |
| | TTGCTTTTTC |
| 1101 | TTCTTCTGTT CCTGCTATGA AGCGGCTCTT TGACAAATAT |
| | GGTTCTGTGG |
| 1151 | TACGGGTAGA TCGTAGGCAG ATTCGTGAGC AGATTCTTTC |
| | GAACACTGAA |
| 1201 | ATCTTAGAAA ATGAGTCAGG GTTCCTCTGC AGTTTGTATG |
| | AATATCCTTT |
| 1251 | ATCCTATTTG ATAGATTGGG CTGTTTTGCT AGACTGTGTT |
| | CGCGGTACCG |
| 1301 | AAATCTCTCT AGAAGATCAG GCCGATTACA CCGTTTGTTT |
| | GCAAGGCTTG |
| 1351 | GATTCTATGT TATCTCAATT TGCGAGTCGT TTACAGTCTG |
| | GACAAAAAGT |
| 1401 | ATTGAATCCT AGAGATGTTT TAAGTGAACA GGCTGCGGTT |
| | ATGCTTGTTC |
| 1451 | ATGGCTTGGC AGCACAGGGC GTGTCGTTTC AAGGATTGAA |
| | AGCTTTGATG |
| 1501 | TATTTGACAG CCGTTCCCCA AAGAATGTGG TTAGGAGCAT |
| | TGCCTTTATT |
| 1551 | TGAATCTTTT CCTGTCTTTA ATCGGATGAA AGAATTTCTT |
| | GGGGAATCTC |
| 1601 | TGGGAGACTA G |

The PSORT algorithm predicts inner membrane (0.6158).

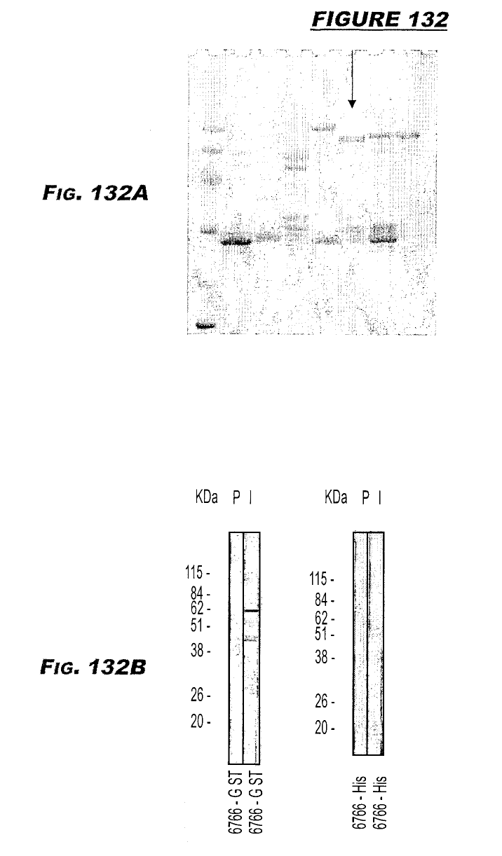

The protein was expressed in E. coli and purified as a GST-fusion product (FIG. 132A) and also as a his-tagged product. The recombinant proteins were used to immunise mice, whose sera were used in a Western blot (FIG. 132B) and for FACS analysis.

These experiments show that cp6766 is a surface-exposed and immunoaccessible protein, and that it is a useful immunogen. These properties are not evident from the sequence alone.

### Example 133

The following C. pneumoniae protein (PID 4376804) was expressed <SEQ ID 265; cp6804>:

| | |
|---|---|
| 1 | MSNQLQPCIS LGCVSYINSF PLSLQLIKRN DIRCVLAPPA |
| | DLLNLLIEGK |
| 51 | LDVALTSSLG AISHNLGYVP GFGIAANQRI LSVNLYAAPT |
| | FFNSPQPRIA |
| 101 | ATLESROSIG LLKVLCRHLW RIPTPHILRF ITTKVLRQTP |
| | ENYDGLLLIG |
| 151 | DAALQHPVLP GFVTYDLASG WYDLTKLPFV FALLLHSTSW |
| | KEHPLPNLAM |
| 201 | EEALQQFESS PEEVLKEAHQ HTGLPPSLLQ EYYALCQYRL |
| | GEEHYESFEK |
| 251 | FREYYGTLYQ QARL |

The cp6804 nucleotide sequence <SEQ ID 266> is:

| | |
|---|---|
| 1 | ATGTCTAACC AACTCCAGCC ATGTATAAGC TTAGGCTGCG |
| | TAAGTTATAT |
| 51 | TAATTCCTTT CCGCTGTCCC TACAACTCAT AAAAAGAAAC |
| | GATATTCGCT |
| 101 | GTGTTCTTGC TCCCCCTGCA GACCTCCTCA ACTTCCTAAT |
| | CGAAGGGAAA |
| 151 | CTCGATGTTG CTTTGACCTC ATCCCTAGGA GCTATCTCTC |
| | ATAACTTGGG |
| 201 | GTATGTCCCC GGCTTTGGAA TTGCAGCAAA CCAACGTATC |
| | CTCAGTGTAA |
| 251 | ACCTCTATGC AGCTCCCACT TTCTTTAACT CACCGCAACC |
| | TCGGATTGCC |
| 301 | GCAACTTTAG AAAGTCGCTC CTCTATAGGA CTCTTAAAAG |
| | TGCTTTGTCG |
| 351 | TCATCTCTGG CGCATCCCAA CTCCTCATAT CCTAAGATTC |
| | ATAACTACAA |
| 401 | AAGTACTCAG ACAAACCCCT GAAAATTATG ATGGCCTCCT |
| | CCTAATCGGA |
| 451 | GATGCAGCGC TACAACATCC TGTACTTCCT GGATTTGTAA |
| | CCTATGACCT |
| 501 | TGCCTCGGGG TGGTATGATC TTACAAAGCT ACCTTTTGTA |
| | TTTGCTCTTC |
| 551 | TTCTACACAG CACCTCTTGG AAAGAACATC CCCTACCCAA |
| | CCTTGCGATG |
| 601 | GAAGAAGCCC TCCAACAGTT CGAATCTTCA CCCGAAGAAG |
| | TCCTTAAAGA |
| 651 | AGCTCATCAA CATACAGGTC TGCCCCCTTC TCTTCTTCAA |
| | GAATACTATG |
| 701 | CCCTATGCCA GTACCGTCTA GGAGAAGAAC ACTACGAAAG |
| | CTTTGAAAAA |
| 751 | TTCCGGGAAT ATTATGGAAC CCTCTACCAA CAAGCCCGAC |
| | TGTAA |

The PSORT algorithm predicts inner membrane (0.060).

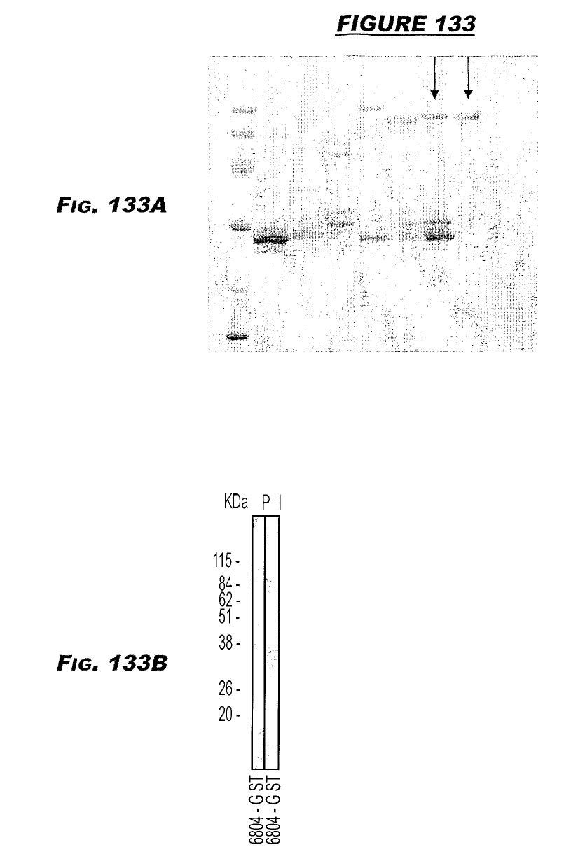

The protein was expressed in E. coli and purified as a GST-fusion product (FIG. 133A). The recombinant protein was used to immunise mice, whose sera were used in a Western blot (FIG. 133B) and for FACS analysis.

These experiments show that cp6804 is a surface-exposed and immunoaccessible protein, and that it is a useful immunogen. These properties are not evident from the sequence alone.

### Example 134

The following C. pneumoniae protein (PID 4376805) was expressed <SEQ ID 267; cp6805>:

| | |
|---|---|
| 1 | MSSLLSCGRI EPTRVTCSLK TYLEDTSQNQ LSTRLVRASV |
| | IFLCALLIIL |
| 51 | VCVALSSIIP SIMALATSFT VAGLILFVMS LLGDVAIISY |
| | LTYSTVTSYR |
| 101 | QNKRAFEIHK PARSVYYEGV RHWDLGRSSL GTGEIPIVRT |
| | LFSPFQNHGL |
| 151 | NHALAAKIFL FMEHFSPEPP NEPLVDWACL IRDFRPHVSS |
| | LCFVIEKQGS |
| 201 | SLRTKEGNTI CEAFRSDYDA HFAMVDCYRL IHSKLIIEKM |
| | GLKNIDIIPS |
| 251 | VMVREDYPSR PGEGYREGLL RMYGGKGAL* |

The cp6805 nucleotide sequence <SEQ ID 268> is:

| | |
|---|---|
| 1 | ATGTCATCAC TACTGAGCTG CGGAAGAATA GAGCCGACTC |
| | GGGTTACCTG |
| 51 | TAGCTTAAAG ACGTATCTTG AGGATACGAG TCAGAATCAG |
| | TTGAGCACAC |
| 101 | GTCTAGTTCG GGCAAGTGTC ATCTTTTTAT GCGCATTGTT |
| | GATCATTTTG |
| 151 | GTTTGTGTGG CCCTCTCTAG TTTGATTCCA AGCATTATGG |
| | CCTTGGCGAC |
| 201 | CTCTTTTACG GTAATGGGGT TAATTCTTTT TGTGATGTCA |
| | CTTCTTGGTG |
| 251 | ACGTTGCAAT TATAAGTTAT CTTACTTATA GCACTGTTAC |
| | GAGTTACCGG |
| 301 | CAAAATAAGA GAGCTTTTGA GATTCACAAG CCCGCTCGCT |
| | CCGTTTACTA |
| 351 | CGAGGGGGTC CGCCATTGGG ATTTAGGACG ATCATCTTTA |
| | GGCACAGGCG |
| 401 | AGATTCCTAT AGTAAGGACG TTATTCTCTC CATTTCAGAA |
| | CCATGGTCTT |
| 451 | AACCATGCCT TAGCTGCTAA AATTTTCCTA TTTATGGAGC |
| | ATTTCAGCCC |
| 501 | TGAGCCACCG AACGAGCCTT TGGTGGATTG GGCCTGTTTG |
| | ATTCGGGATT |
| 551 | TTAGGCCTCA CGTCAGTTCT TTGTGCTTTG TTATTGAAAA |
| | ACAAGGGTCA |
| 601 | TCGCTGAGGA CTAAGGAAGG CAATACGATT TGTGAGGCTT |
| | TCCGCTCTGA |
| 651 | TTACGACGCC CATTTTGCTA TGGTAGATTG CTACCGGTTG |
| | ATCCACTCTA |
| 701 | AGTTGATTAT AGAGAAAATG GGATTGAAGA ATATCGATAT |
| | CATTCCGAGT |
| 751 | GTCATGGTTC GTGAAGATTA TCCTAGCCGT CCTGGGGAGG |
| | GCTATCGCGA |
| 801 | AGGCCTATTA CGTATGTATG GTGGCAAGGG GGCTCTGTGA |

The PSORT algorithm predicts inner membrane (0.711).

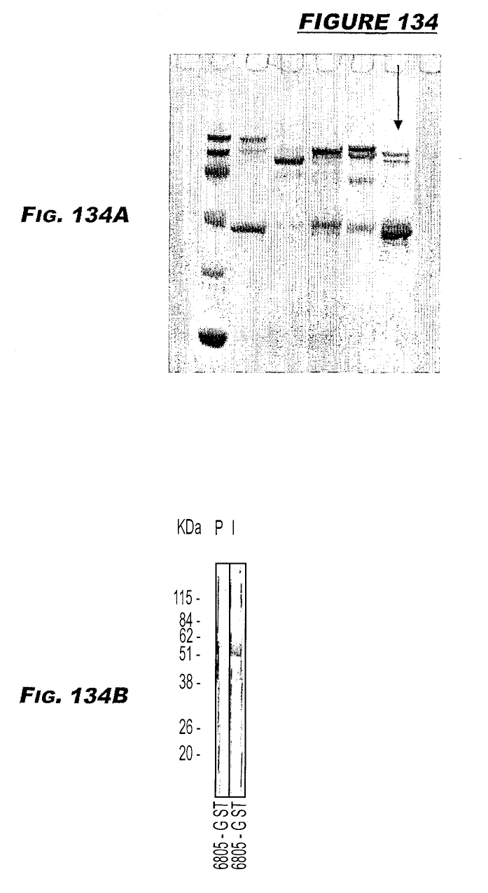

The protein was expressed in E. coli and purified as a GST-fusion product (FIG. 134A). The recombinant protein was used to immunise mice, whose sera were used in a Western blot (FIG. 134B) and for FACS analysis.

These experiments show that cp6805 is a surface-exposed and immunoaccessible protein, and that it is a useful immunogen. These properties are not evident from the sequence alone.

### Example 135

The following C. pneumoniae protein (PID 4376813) was expressed <SEQ ID 269; cp6813>:

| | |
|---|---|
| 1 | MSGPSRTESS QVSVLSYVPR DKEIAPKKQF TIAKISTLAI |
| | LASLALGALV |
| 51 | AGISLTIVLG NPVFLALLIT TALFSVVTFL VYHQMTSKVS |
| | SNWQKVLEQN |
| 101 | FKPLGKAWQE KNVDCYSNEM QFYNNHLNPK FKVAIQTDAS |
| | QPFQPTFLTG |
| 151 | LRVIEKNQST GIIFNPVGPT NLIDNTATNL STILYSTLKD |
| | KSVWDTCKQR |
| 201 | EGGPAKGEDP ESPTEVRVVK LPNEALDQTF NLNLSSAEKK |
| | SILPTFLGHV |
| 251 | CGPKSEELPN QQEYYRQALL AYENCLKAAI ESHAAIVALP |
| | LFTSVYEVPP |
| 301 | EEILPKEGTF YWDNQTQAFC KRALLDAIQN TALRYPQRSL |
| | LVILQDPFNT |
| 351 | IESQSRSEE* |

The cp6813 nucleotide sequence <SEQ ID 270> is:

| | |
|---|---|
| 1 | ATGTCAGGAC CCTCACGTAC TGAGAGCTCT CAAGTTTCTG |
| | TACTATCCTA |
| 51 | TGTGCCTCGG GATAAAGAAA TTGCTCCTAA AAAACAGTTT |
| | ACCATAGCAA |
| 101 | AAATATCCAC TCTTGCAATC CTAGCTTCTT TAGCTTTAGG |
| | AGCTTTGGTG |
| 151 | GCTGGAATCT CTTTAACGAT AGTATTAGGG AACCCTGTAT |
| | TTTTGGCTCT |
| 201 | TCTCATTACC ACGGCCCTCT TCTCAGTTGT AACCTTCTTA |
| | GTCTACCACC |
| 251 | AAATGACCTC AAAGGTATCT TCTAACTGGC AGAAAGTTCT |
| | AGAGCAAAAC |
| 301 | TTCAAGCCTT TGGGAAAAGC GTGGCAAGAA AAAAACGTAG |
| | ACTGCTACTC |
| 351 | AAACGAGATG CAATTTTACA ATAATCACCT GAACCCTAAG |
| | TTCAAGGTAG |
| 401 | CGATACAAAC AGATGCGTCT CAACCATTTC AGCCTACTTT |
| | CTTAACTGGA |
| 451 | CTTAGAGTGA TCGAAAAAAA TCAATCCACA GGGATCATCT |
| | TTAATCCCGT |
| 501 | AGGCCCAACG AATCTGATCG ACAACACTGC AACGAACCTC |
| | TCTACTATCC |
| 551 | TTTACTCCAC CCTAAAAGAT AAAAGCGTGT GGGATACATG |
| | CAAGCAACGC |
| 601 | GAAGGGGGTC CCGCAAAAGG AGAAGACCCC TTTTCCCCTA |
| | CCGAAGTGAG |
| 651 | AGTAGTAAAA CTTCCAAACG AAGCTCTAGA TCAAACGTTT |
| | AATCTAAATT |
| 701 | TAAGCTCTGC AGAAAAGAAA AGTATTCTTC CGACCTTTTT |
| | AGGCCACGTA |
| 751 | TGCGGCCCTA AATCTGAAGA GTTACCAAAT CAGCAAGAAT |
| | ATTATCGCCA |
| 801 | AGCTTTACTA GCGTACGAGA ACTGCCTTAA AGCAGCTATA |
| | GAAAGTCATG |
| 851 | CAGCAATCGT TGCTCTTCCT CTCTTTACTT CGGTCTATGA |
| | AGTGCCTCCA |
| 901 | GAAGAGATTC TTCCTAAAGA AGGCACTTTC TATTGGGACA |
| | ACCAAACTCA |
| 951 | AGCGTTTTGC AAACGCGCTT TATTGGACGC TATTCAAAAT |
| | ACGGCCCTAC |
| 1001 | GCTATCCTCA AAGATCCTTA CTTGTTATAC TCCAAGATCC |
| | TTTTAATACT |
| 1051 | ATAGAATCAC AAAGTCGTTC TGAGGAGTAA |

The PSORT algorithm predicts inner membrane (0.4291).

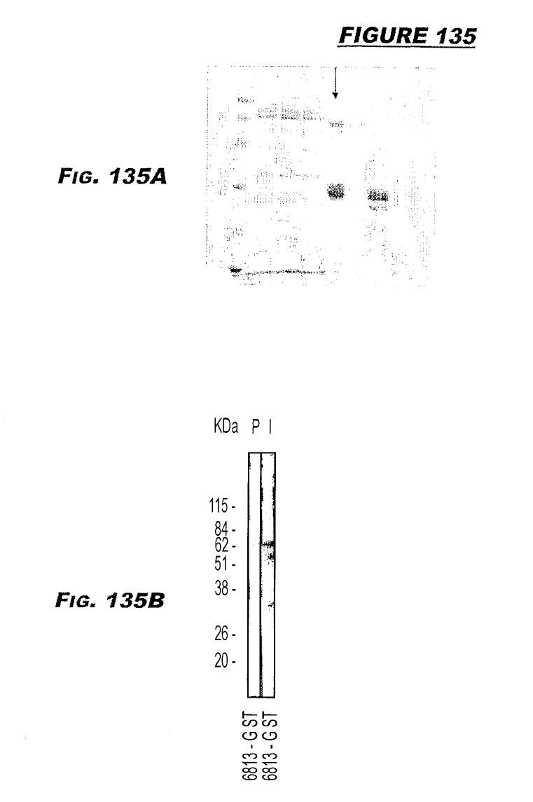

The protein was expressed in E. coli and purified as a GST-fusion product (FIG. 135A). The recombinant protein was used to immunise mice, whose sera were used in a Western blot (FIG. 135B) and for FACS analysis.

These experiments show that cp6813 is a surface-exposed and immunoaccessible protein, and that it is a useful immunogen. These properties are not evident from the sequence alone.

### Example 136

The following C. pneumoniae protein (PID 4376844) was expressed <SEQ ID 271; cp6844>:

| | |
|---|---|
| 1 | MWRVVLRFLI IFILGRAVFP LRASESFSWE TSTCLTVLGI |
| | PFIDIILTIN |
| 51 | EDFVAQCGLQ IGTISSTNNA KIKEIFLIYK EKFPEASISF |
| | KRKEPLNLSQ |
| 101 | SHLSDLGILC MRNGETYAEG MANKENGPAL KQPKDLRLVL |
| | RCPNQPDTLL |
| 151 | YSEKEAEKGI ETNTCLCNQG YTLLDGQLIL YGDSIEKFLK |
| | ETKRKNNHTL |
| 201 | VDLCDSQVVT TFLGRFWSLL NYVQVLFLSE DSAKILAGIP |
| | DLAQATQLLS |
| 251 | HTVPLLFIYT NDSIHIIEQG KESSFTYNQD LTEPILGFLF |
| | GYINRGSMEY |
| 301 | CFNCAQSSLG ET* |

The cp6844 nucleotide sequence <SEQ ID 272> is:

| | |
|---|---|
| 1 | ATGTGGCGCG TTGTCCTCAG ATTCCTTATA ATTTTTATCT |
| | TGGGAAGAGC |
| 51 | CGTCTTCCCT CTAAGAGCTT CAGAAAGCTT CTCCTGGGAA |
| | ACATCGACCT |
| 101 | GTTTACCAGT GCTAGGGATT CCTTTCATAG ATATTATCCT |
| | CACAACGAAT |
| 151 | GAGGACTTTG TTGCCCAGTG CGGCCTGCAA ATAGGAACCA |
| | TTTCTTCGAC |
| 201 | TAATAACGCA AAAATAAAAG AAATTTTTTT GATATATAAG |
| | GAAAAATTTC |
| 251 | CAGAAGCCTC TATCAGTTTC AAACGAAAAG AACCTCTAAA |
| | CCTTTCCCAA |
| 301 | TCCCATCTCT CCGATTTAGG TATTTTATGT ATGCGTAACG |
| | GAGAAACTTA |
| 351 | CGCTGAGGGA ATGGCAAATA AAGAAAACGG ACCCGCTCTA |
| | AAACAACCCA |
| 401 | AGGATCTAAG ATTAGTTTTA CGTTGTCCTA ACCAACCAGA |
| | TACCCTGCTC |
| 451 | TACTCGGAAA AAGAAGCAGA AAAGGGCATA GAAACAAATA |
| | CTTGCCTATG |
| 501 | CAATCAGGGA TACACACTCC TGGATGGGCA ATTGATTCTC |
| | TACGGGGATA |
| 551 | GTATAGAAAA GTTTCTGAAA GAGACCAAAA GAAAGAATAA |
| | CCACACGCTT |
| 601 | GTTGATCTTT GTGACTCACA AGTCGTGACC ACGTTCCTCG |
| | GTCGCTTTTG |
| 651 | GTCTCTTCTA AACTACGTTC AAGTTCTTTT CCTATCTGAA |
| | GACTCCGCTA |
| 701 | AAATTCTTGC GGGCATCCCA GACCTAGCTC AAGCTACGCA |
| | ATTGCTTTCC |
| 751 | CACACCGTAC CTTTGCTTTT TATTTATACC AACGATTCTA |
| | TTCACATCAT |
| 801 | AGAACAAGGC AAAGAAAGTA GTTTTACCTA TAACCAAGAT |
| | TTAACAGAGC |
| 851 | CCATTTTAGG ATTTCTCTTT GGTTACATAA ATCGCGGCTC |
| | TATGGAATAC |
| 901 | TGCTTTAATT GTGCACAGTC TTCATTAGGA GAAACCTAA |

The PSORT algorithm predicts inner membrane (0.1786).

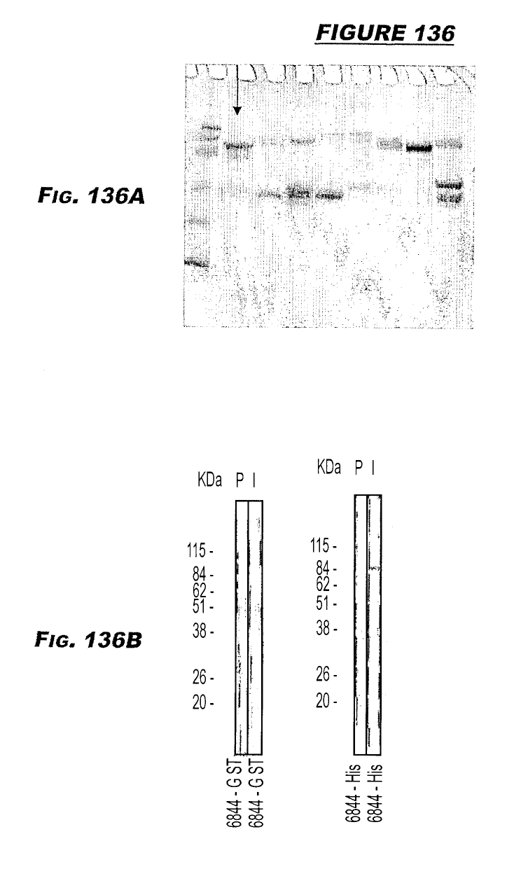

The protein was expressed in E. coli and purified as a GST-fusion product (FIG. 136A) and also in his-tagged form. The recombinant proteins were used to immunise mice, whose sera were used in a Western blot (FIG. 136B) and for FACS analysis.

These experiments show that cp6844 is a surface-exposed and immunoaccessible protein, and that it is a useful immunogen. These properties are not evident from the sequence alone.

### Example 137

The following C. pneumoniae protein (PID 4377201) was expressed <SEQ ID 273; cp7201>:

| | |
|---|---|
| 1 | VLVGICRSLY PEHPRSFYYR VSGDIGSRFD DRGFVNSGVE |
| | TLPYSSGSFG |
| 51 | IFWISFTDPT FNFAIVNTFM RTAGINEVSR PMTQDTETSL |
| | IEMRDLSEQQ |
| 101 | EANNTDSLEQ EESLMGIVGH TVGGVSMTVT SSPNIFYRIQ |
| | TLLGLPETLA |
| 151 | EAEENPTFPN STIDDLAEIM NNLVRISDAV SIFWIFPIVD |
| | TTYNGVLLAV |
| 201 | CIGFFGINGI CSTFLMLTNP RSRRDRWRNL RIMVLCYRSL |
| | GSGMNLFDLS |
| 251 | NNVRMAARRH VTSCTVALYA MVTLFGWTVA IQDALQYGFP |
| | SVRDAFYRYC |
| 301 | LRHRYCLTQR NEDSLQTTGT RFQVTRTHLE DQQMVASILN |
| | LSVFGLFFGF |
| 351 | VGLMTTFGGL EISPSCRWDA ANNRTVGIF* |

The cp7201 nucleotide sequence <SEQ ID 274> is:

| | |
|---|---|
| 1 | GTGCTCGTTG GTATCTGTCC TTCTCTATAT CCAGAACATC |
| | CTCGCTCCTT |
| 51 | TTATTATCGT GTTTCTGGAG ATATAGGCTC CCGATTCGAC |
| | GATAGAGGAT |
| 101 | TTGTAAACTC TGGAGTCGAA ACCCTGCCAT ACTCTTCAGG |
| | CAGCTTTGGG |
| 151 | ATTTTTTGGA TCTCGTTTAC GGATCCCACA TTTAATTTTG |
| | CTATCGTAAA |
| 201 | TACCTTTATG CGAACTGCAG GGATCAATGA AGTCTCTAGA |
| | CCCATGACAC |
| 251 | AAGATACAGA AACTTCATTG ATAGAAATGA GAGACCTAAG |
| | TGAACAACAA |
| 301 | GAAGCGAATA ACACAGATTC TTTAGAGCAA GAAGAGAGCT |
| | TAATGGGTAT |
| 351 | TGTAGGACAT ACTGTGGGAG GAGTTTCCAT GACCGTGACC |
| | TCCAGTCCAA |
| 401 | ATATCTTTTA TCGTATACAA ACACTTCTGG GACTGCCAGA |
| | GACTCTTGCA |
| 451 | GAAGCTGAAG AAAATCCTAC CTTCCCAAAT TCTACTATAG |
| | ATAGCCTTGC |
| 501 | AGAAATAATG ATGAACCTCG TAAGGATCTC TGATGCTGTC |
| | TCTATTTTCT |
| 551 | GGATTTTTCC TATCGTAGAT ACTACATATA ATGGAGTTTT |
| | ATTAGCCGTC |
| 601 | TGTATCGGCT TCTTCGGAAT CAATGGGATT TGTTCCACGT |
| | TCCTTATGCT |
| 651 | TACGAATCCA CGCTCTCGTC GAGATAGATG GAGGAATTTA |
| | CGCATCATGG |
| 701 | TTCTTTGCTA TCGTTCTTTG GGAAGCGGAA TGAATCTCTT |
| | TGATCTTAGC |
| 751 | AATAATGTGC GCATGGCAGC ACGTAGGCAT GTGACATCAT |
| | GTACAGTAGC |
| 801 | TCTCTATGCT ATGGTCACTC TATTTGGATG GACAGTAGCA |
| | ATACAAGATG |
| 851 | CTTTGCAATA TGGTTTCCCT AGCGTTCGGG ATGCCTTCTA |
| | TAGATATTGC |
| 901 | TTACGCCACA GATATTGCTT AACTCAAAGA AACGAAGACT |
| | CTCTGCAAAC |
| 951 | TACAGGAACG CGCTTTCAGG TTACCCGTAC ACATCTAGAA |
| | GATCAACAGA |
| 1001 | TGGTGGCTTC TATTTTGAAT TTGAGTGTTT TTGGGCTCTT |
| | TTTTGGATTC |
| 1051 | GTAGGGCTAA TGACCACGTT TGGAGGATTA GAAATCTCAC |
| | CATCTTGTCG |
| 1101 | GTGGGATGCA GCAAATAACC GAACGGTAGG TATTTTTTAG |

The PSORT algorithm predicts inner membrane (0.3102).

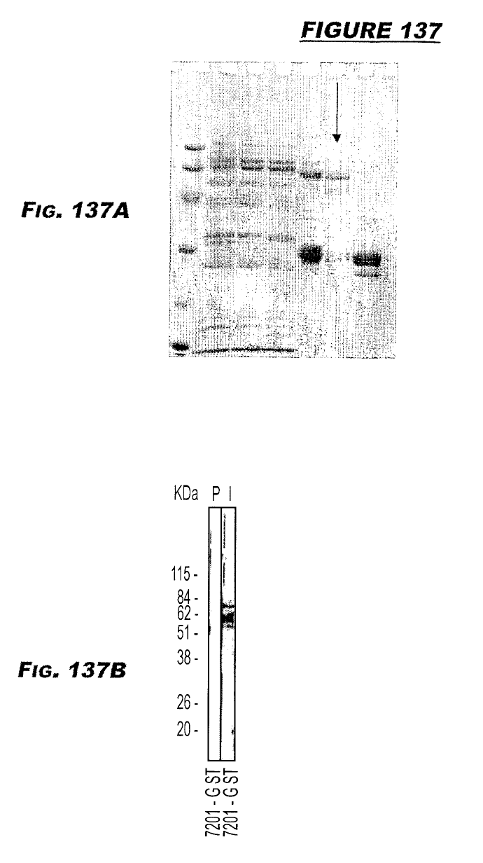

The protein was expressed in E. coli and purified as a GST-fusion product (FIG. 137A). The recombinant protein was used to immunise mice, whose sera were used in a Western blot (FIG. 137B) and for FACS analysis.

These experiments show that cp7201 is a surface-exposed and immunoaccessible protein, and that it is a useful immunogen. These properties are not evident from the sequence alone.

### Example 138

The following C. pneumoniae protein (PID 4377251) was expressed <SEQ ID 275; cp7251>:

| | |
|---|---|
| 1 | MAPIHGSNAF VEDILHSHPS PQATYFSSTR AQKLHEFKDR |
| | HPVLTRIASV |
| 51 | IIKIFKVLIG LIILPLGIYW LCQTLCTNSI LPSKNLLKIF |
| | KKQPNTKTLK |
| 101 | TNYLHALQDY SSKNRVASMR RVPILQDNVL IDTLEICLSQ |
| | APTNRWMLIS |
| 151 | LGSDCSLEEI ACKEIFDSWQ RFAKLIGANI LVYNYPGVMS |
| | STGSSSLKDL |
| 201 | ASAHNICTRY LKDKEQGPGA KEIITYGYSL GGLIQAEALR |
| | DQKIVANDDT |
| 251 | TWIAVKDRCP LFISPEGFHS CRRIGKLVAR LFGWGTKAVE |
| | RSQDLPCLEI |
| 301 | FLYPTDSLRR STVRQNKLLA PELTLAHAIK NSPYVQNKEF |
| | IEVRLSSDID |
| 351 | PIDSKTRVAL ATPILKKLS* |

The cp7251 nucleotide sequence <SEQ ID 276> is:

| | |
|---|---|
| 1 | ATGGCTCCAA TTCACGGAAG TAATGCGTTT GTTGAGGATA |
| | TTTTACATTC |
| 51 | CCACCCTTCT CCACAAGCGA CTTATTTTTC TTCAACACGC |
| | GCCCAAAAAC |
| 101 | TTCATGAGTT TAAAGACAGG CATCCCGTGC TTACACGGAT |
| | TGCTTCTGTA |
| 151 | ATTATTAAAA TTTTTAAAGT TCTGATAGGG CTGATCATCC |
| | TTCCCTTAGG |
| 201 | AATCTACTGG CTATGTCAAA CGCTTTGTAC AAACTCGATT |
| | CTCCCTTCCA |
| 251 | AGAATTTATT AAAAATTTTC AAGAAGCAAC CCAACACTAA |
| | AACCTTAAAA |
| 301 | ACTAATTATT TGCATGCTTT GCAAGATTAT TCCTCGAAAA |
| | ACCGCGTTGC |
| 351 | TTCCATGAGA CGAGTTCCTA TCCTCCAGGA TAATGTTCTC |
| | ATCGACACTT |
| 401 | TGGAAATATG CCTTTCACAA GCACCTACGA ATCGTTGGAT |
| | GCTCATTTCT |
| 451 | TTAGGAAGTG ACTGTAGCTT GGAAGAAATC GCTTGTAAGG |
| | AGATCTTTGA |
| 501 | TTCTTGGCAA AGATTTGCCA AGTTGATAGG GGCCAATATA |
| | CTCGTTTATA |
| 551 | ACTACCCCGG AGTCATGTCC AGCACAGGGA GCAGCAGCCT |
| | AAAGGACCTA |
| 601 | GCATCAGCTC ATAATATTTG TACAAGATAC CTTAAAGATA |
| | AAGAACAGGG |
| 651 | CCCTGGAGCA AAAGAAATCA TTACCTATGG GTACTCCCTA |
| | GGAGGTTTGA |
| 701 | TACAAGCAGA AGCATTGCGA GACCAGAAGA TTGTTGCAAA |
| | CGATGATACT |
| 751 | ACTTGGATAG CAGTCAAAGA TAGGTGTCCT CTCTTTATAT |
| | CTCCAGAAGG |
| 801 | TTTCCACAGT TGCAGACGCA TAGGAAAGCT AGTAGCTCGT |
| | CTTTTTGGCT |
| 851 | GGGGGACCAA AGCCGTAGAG AGAAGCCAAG ACCTTCCCTG |
| | CCTAGAAATT |
| 901 | TTTCTCTATC CTACGGATTC CTTACGAAGA TCAACAGTCA |
| | GACAGAACAA |
| 951 | GCTCTTAGCA CCTGAACTTA CTCTCGCTCA TGCGATAAAA |
| | AATAGTCCCT |
| 1001 | ATGTTCAAAA TAAAGAATTT ATAGAAGTAC GATTATCGTC |
| | TGATATCGAT |
| 1051 | CCCATCGACA GCAAAACAAG AGTGGCTCTT GCCACACCAA |
| | TTTTGAAAAA |
| 1101 | GCTCTCTTAG |

The PSORT algorithm predicts inner membrane (0.4545).

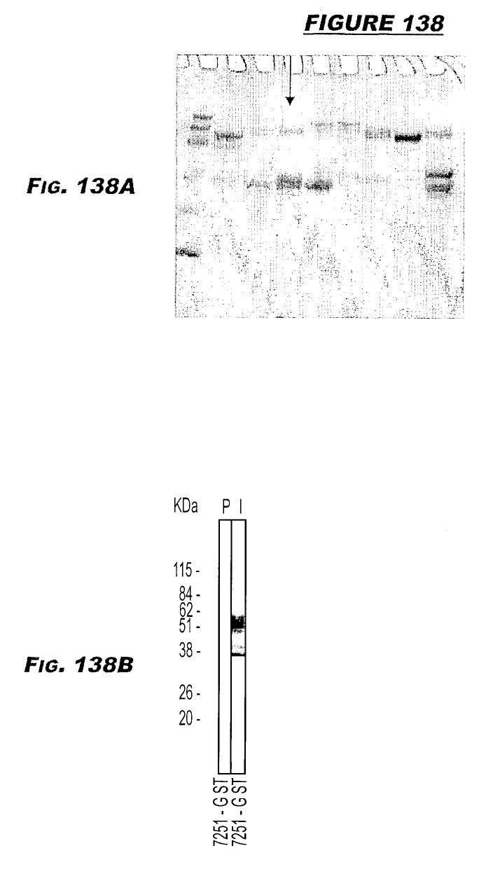

The protein was expressed in E. coli and purified as a GST-fusion product (FIG. 138A). The recombinant protein was used to immunise mice, whose sera were used in a Western blot (FIG. 138B) and for FACS analysis.

These experiments show that cp7251 is a surface-exposed and immunoaccessible protein, and that it is a useful immunogen. These properties are not evident from the sequence alone.

### Example 139

The following C. pneumoniae protein (PID 4377288) was expressed <SEQ ID 277; cp7288>:

| | |
|---|---|
| 1 | MHMSNPISLP SPAELIAKYN LIPKTSPIYP RRTELIILEE |
| | NACQTRLTNV |
| 51 | AQVLHPSSLF SMSKKILNPC GCSGGPLCWV ILNILAFIIT |
| | SVLFIILLPV |
| 101 | NLIVAGLRLF MPLPPKKIVE DLSEPTTEET NEVIQPFIFA |
| | LQALLFEDNK |
| 151 | LRSFKIVEQS VGKAPLPNPF LNRLVAISPQ ESQEAMRKIP |
| | DLCSQLKKVL |
| 201 | KSLGVLTPEW KHNLKYFEGL KNEHDSNPKD KTFPILIKLL |
| | IEALTGKSSL |
| 251 | PKTPSTKERM QAALFIASSC KTCKPTWGEV ITRSLNRLYS |
| | IANEGDNQLL |
| 301 | IWVQEFKERE LMSIQDGDDA EEYRFAAQQH GERYTEAIEQ |
| | VLRNESAAKL |
| 351 | QWHVINTMKF FHGKNLGLVT EHLQDTLGAL TLRQTTVDTH |
| | QGREDADLSA |
| 401 | ALFLNKYLNS GNQLVNSVFK SMQKADPETK ALIREFALDI |
| | LYASLRLPQT |
| 451 | SAHTEVFSTL LMDPETYEPN KACIAYLLYV LKIIEL* |

The cp7288 nucleotide sequence <SEQ ID 278> is:

| | |
|---|---|
| 1 | ATGCATATGT CTAACCCCAT CTCTTTGTTT TCCCCTGCAG |
| | AGTTAATAGC |
| 51 | AAAGTACAAT TTAATTCCAA AAACTTCGCC GATTTATCCT |
| | CGGAGGACGG |
| 101 | AACTTATTAT CTTGGAAGAA AATGCGTGTC AAACACGCCT |
| | AACCAACGTG |
| 151 | GCTCAGGTCC TACATCCTTC TAGCCTATTC AGTATGTCAA |
| | AAAAAATACT |
| 201 | GAATCCCTGC GGGTGCTCTG GTGGTCCCTT ATGTTGGGTG |
| | ATTCTCAACA |
| 251 | TCCTAGCATT TATTATTACT TCAGTACTGT TTATCATTCT |
| | TTTACCGGTG |
| 301 | AATCTCATCG TAGCAGGTCT TCGTCTCTTC ATGCCTCTTC |
| | CCCCTAAAAA |
| 351 | AATCGTAGAG GATTTAAGTG AACCTACTAC TGAAGAAACG |
| | AATGAGGTCA |
| 401 | TTCAACCCTT CATTTTCGCT TTGCAAGCGT TGCTTTTTGA |
| | GGATAACAAA |
| 451 | CTTCGCTCTT TTAAAATTGT TGAACAAAGT GTAGGCAAAG |
| | CACCCTTACC |
| 501 | TAATCCCTTT TTAAATAGAC TAGTAGCAAT TTCGCCGCAA |
| | GAAAGCCAAG |
| 551 | AAGCCATGCG GAAGATTCCG GATCTATGCT CACAACTGAA |
| | AAAAGTATTA |
| 601 | AATGCTCTAG GCGTGCTAAC TCCAGAATGG AAGCACATGC |
| | TGAAGTACTT |
| 651 | TGAGGGACTG AAAAACGAAC ATGATAGTAA TCCTGATAAA |
| | AAGACGTTCC |
| 701 | CAATATTGAT CAAGCTCCTC ATAGAAGCTC TTACTGGAAA |
| | GTCCTCTTTA |
| 751 | CCCAAAACTC CTAGTACAAA GGAAAAAATG CAAGCGGCCT |
| | TATTTATTGC |
| 801 | AAGTTCTTGC AAGACTTGTA AGCCGACTTG GGGAGAAGTC |
| | ATAACCAGAT |
| 851 | CTCTTAACAG ACTCTATAGT ATAGCTAATG AAGGAGACAA |
| | TCAGCTTCTG |
| 901 | ATTTGGGTTC AAGAGTTTAA AGAACGAGAG CTGATGTCCA |
| | TCCAAGATGG |
| 951 | TGATGATGCT GAAGAGTATC GGTTTGCGGC TCAGCAACAC |
| | GGTGAGCGTT |
| 1001 | ACACAGAGGC AATAGAACAA GTTCTACGAA ACGAGTCAGC |
| | AGCCAAACTA |
| 1051 | CAATGGCATG TGATCAACAC TATGAAATTC TTCCATGGGA |
| | AAAATCTCGG |
| 1101 | TCTAGTTACA GAACACCTAC AAGATACTCT CGGCGCCCTA |
| | ACTTTACGTC |
| 1151 | AAACTACAGT GGACACACAT CAAGGCAGAG AAGACGCTGA |
| | TTTGTCAGCT |
| 1201 | GCTCTTTTCC TAAATAAGTA TTTAAATTCT GGAAATCAAC |
| | TTGTTAATAG |
| 1251 | CGTCTTTAAA TCCATGCAAA AAGCAGATCC AGAAACCAAA |
| | GCTTTAATCC |
| 1301 | GTGAGTTTGC TCTAGATATA TTATATGCAT CCTTACGGCT |
| | TCCTCAAACT |
| 1351 | TCCGCTCATA CCGAGGTCTT TTCTACACTC TTAATGGACC |
| | CAGAGACCTA |
| 1401 | TGAACCTAAT AAAGCTTGTA TCGCCTACTT GCTCTATGTA |
| | TTAAAGATCA |
| 1451 | TCGAACTATA A |

The PSORT algorithm predicts inner membrane (0.5989).

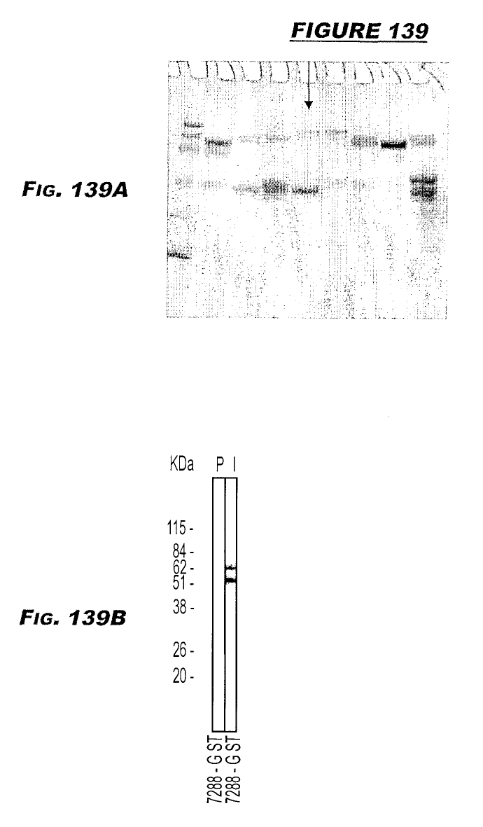

The protein was expressed in E. coli and purified as a GST-fusion product (FIG. 139A). The recombinant protein was used to immunise mice, whose sera were used in a Western blot (FIG. 139B) and for FACS analysis.

These experiments show that cp7288 is a surface-exposed and immunoaccessible protein, and that it is a useful immunogen. These properties are not evident from the sequence alone.

### Example 140

The following C. pneumoniae protein (PID 4377359) was expressed <SEQ ID 279; cp7359>:

```
    1 MPGSVSSPPL SPVIVRERVP SSSGSDLIQP HAVLKISILI FALVTILGIV
  51 LVVLSSALGA IPSLVLTVSG CIAIAVGLIG LGILVTRLIL STIRKVDAMG
101 YDAAVKEEQY ISRIRELESE NREIRDRNRA VEDQCAHLSE ENKDLRDPFY
151 LHGMTERLIA SLEIENQALV AENILLKDWN ASLSRDFRAY KQKFPLGALE
201 PWKFDIACIM EQNLFLKPEC IAMVKSLPLE TQRLFLYPKG FQSLVNRFAP
251 RSRFFQTPKY EYNSRNENED GKVAAVCARL KKEFFSAVLG ACSYEELGGI
301 CERAVALKET LPLPEAVYDT LVQEFPNLLT AESLWKEWCF YSYPYLRPYL
351 SVDYCKRIFV QLFEELCLKL FTTGSPEDQA LVRLFSYYRN HIPAVLASFG
401 LPPPETGGSV FVLLPKQENL LWSQIEVLAT RYLKDTFVRN SEWTGSFEMM
451 FSYNEMCKEI SEGRIRFAED YETRHSEEFP PSPLSEEGEG EEFLPPCSEE
501 EVSVLERPDL DVDSMWVWHP PVPKGPL*
```

The cp7359 nucleotide sequence <SEQ ID 280> is:

```
      1 ATGCCAGGTT CTGTGTCATC ACCTCCTTTG TCTCCTGTAA TTGTCCGTGA
    51 AAGGGTCCCA TCCTCTTCAG GATCCGACCT CATACAGCCT CATGCTGTTT
  101 TAAAGATCTC CATCCTAATT TTTGCGCTTG TGACAATTTT AGGAATTGTT
  151 CTTGTAGTGT TGTCTAGTGC TTTAGGAGCT CTTCCTAGTT TAGTTTTGAC
  201 GGTTTCTGGT TGTATTGCAA TAGCTGTAGG CCTGATTGGT TTAGGGATTC
  251 TTGTGACACG GCTGATTCTC TCTACGATCA GAAAAGTAGA TGCCATGGGT
  301 TATGATGCTG CGGTCAAAGA AGAGCAGTAT TTGTCACGTA TCAGAGAATT
  351 AGAGTCTGAA AATAGAGAGA TTAGAGATAG AAATCGTGCT GTCGAAGATC
  401 AGTGTGCCCA TTTATCCGAA GAGAACAAGG ACCTTAGGGA TCCCGAATAT
  451 CTACATGGAA TGACTGAAAG GCTCATTGCG AGCTTAGAAA TAGAGAATCA
  501 AGCTCTCGTA GCTGAGAACA TTCTTCTCAA AGACTGGAAT GCAAGCCTAT
  551 CTAGAGATTT CCGCGCATAT AAGCAAAAAT TTCCTCTTGG GGCATTAGAA
  601 CCCTGGAAAG AAGATATTGC ATGTATCATG GAACAAAATC TCTTTTTAAA
  651 ACCGGAATGT ATCGCGATGG TTAAGTCTCT TCCATTAGAG ACGCAACGGC
  701 TGTTTTTATA TCCAAAAGGA TTTCAGTCTT TAGTTAATCG ATTTGCTCCG
  751 CGGTCTCGCT TTTTCCAGAC TCCAAAGTAT GAATATAACA GTAGGAATGA
  801 AAATGAGGAC GGAAAGGTAG CCGCAGTGTG CGCCCGTTTG AAAAAAGAAT
  851 TCTTCAGTGC TGTTTTAGGA GCCTGTAGTT ACGAAGAACT AGGGGGCATT
  901 TGTGAAAGAG CAGTAGCACT TAAAGAGACG TTGCCATTGC CTGAAGCTGT
  951 CTATGATACC CTAGTTCAGG AGTTCCCAAA TCTTCTTACT GCTGAGAGTT
1001 TATGGAAAGA ATGGTGCTTC TATTCCTATC CCTACCTTCG TCCCTATCTT
1051 TCTGTGGATT ACTGTAAGAG GTTATTTGTA CAACTTTTTG AGGAACTCTG
1101 CCTAAAGCTT TTTACAACGG GATCTCCAGA AGACCAAGCT TTGGTTCGCC
1151 TTTTCTCTTA CTATAGGAAT CATATTCCCG CAGTCTTGGC CTCATTTGGT
1201 TTGCCCCCGC CTGAGACAGG GGGGTCTGTA TTTGTATTGC TACCAAAACA
1251 AGAAAACCTT CTTTGGAGTC AAATTGAGGT GCTGGCTACA AGGTATCTCA
1301 AAGATACCTT CGTGAGAAAC TCAGAATGGA CGGGCTCTTT CGAGATGATG
1351 TTTTCTTATA ACGAGATGTG TAAGGAGATC TCCGAAGGAA GGATTCGTTT
1401 TGCTGAAGAC TATGAAACGA GGCATTCCGA AGAATTCCCT CCTTCCCCTC
1451 TCTCTGAAGA AGGAGAGGGC GAAGAATTCC TTCCTCCTTG CTCTGAAGAA
1501 GAGGTTTCGG TTCTTGAGCG CCCAGATCTA GATGTAGACT CTATGTGGGT
1551 CTGGCATCCG CCGGTCCCTA AGGGACCTCT TTAA
```

The PSORT algorithm predicts inner membrane (0.7453).

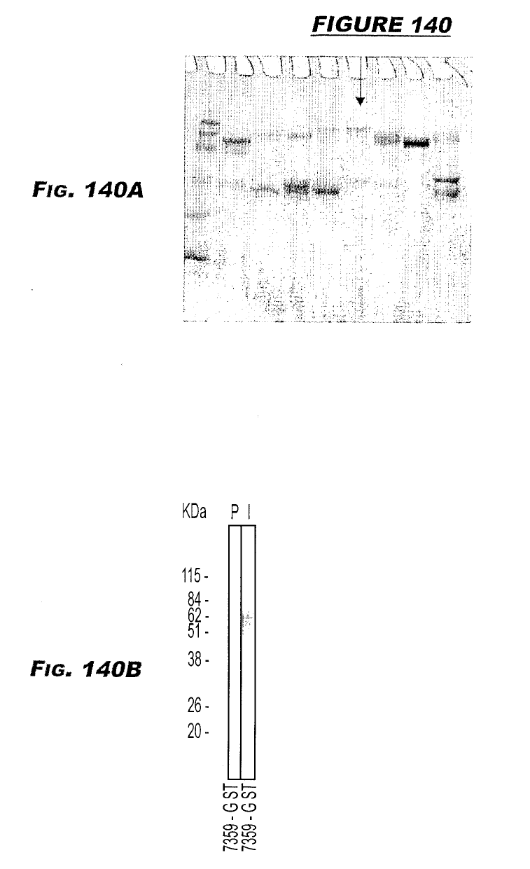

The protein was expressed in E. coli and purified as a GST-fusion product (FIG. 140A). The recombinant protein was used to immunise mice, whose sera were used in a Western blot (FIG. 140B) and for FACS analysis.

These experiments show that cp7359 is a surface-exposed and immunoaccessible protein, and that it is a useful immunogen. These properties are not evident from the sequence alone.

### Example 141

The following C. pneumoniae protein (PID 4377374) was expressed <SEQ ID 281; cp7374>:

```
    1 MDKQSSGNSG CIWHPFTQSA LDSTPIKIVR GEGAYLYAES GTRYLDAISS
  51 WWCNLHGHGH FYITKKLCEQ AQKLEHVIFA NFTHEPALEL VSKLAPLLPE
101 GLERFFFSDN GSTSIEIAMK IAVQYYYNQN KAKSHFVGLS NAYHGDTFGA
151 MSIAGTSPTT VPFHDLFLPS STIAAPYYGK EELAIAQAKT VFSESNIAAF
201 IYEPLLQGAG GMLMYNPEGL KEILKLAKHY GVLCIADEIL TGFGRTGPLF
251 ASEFTDIPPD IICLSKGLTG GYLPLALTVT TKEIHDAFVS QDRMKALLHG
301 HTFTGNPLGC SAALASLDLT LSPECLQQRQ MIERCHQEFQ EAHGSLWQRC
351 EVLGTVLALD YPAEATGYFS QYRDHLNRFF LERGVLLRPL GNTLYVLPPY
401 CIQEEDLRII YSHLQDALCL QPQ*
```

The cp7374 nucleotide sequence <SEQ ID 282> is:

```
      1 ATGGACAAGC AATCATCAGG GAATTCAGGG TGTATCTGGC ACCCCTTCAC
    51 TCAATCTGCA TTAGATTCTA CACCCATAAA GATTGTAAGG GGAGAAGGTG
  101 CTTACCTCTA TGCGGAATCA GGAACAAGAT ATCTTGATGC GATATCTTCA
  151 TGGTGGTGCA ACCTCCACGG TCATGGGCAT CCCTACATTA CAAAAAAATT
  201 ATGTGAGCAA GCACAGAAGT TAGAACATGT GATCTTCGCA AATTTCACCC
  251 ATGAACCGGC TCTAGAGCTC GTATCGAAAC TCGCTCCCCT CCTTCCTGAA
  301 GGTCTAGAAC GTTTCTTTTT CTCTGACAAC GGATCAACGT CTATCGAAAT
  351 AGCAATGAAA ATTGCTGTGC AATATTACTA CAATCAAAAC AAGGCTAAGA
  401 GCCATTTTGT TGGACTCAGC AATGCCTATC ACGGAGATAC ATTTGGAGCT
  451 ATGTCGATAG CTGGCACGAG CCCTACTACA GTTCCCTTTC ATGATCTTTT
  501 TCTTCCTTCC AGTACAATTG CTGCTCCCTA TTATGGCAAG GAAGAGCTTG
  551 CCATTGCCCA AGCAAAAACA GTCTTTTCTG AAAGCAATAT CGCAGCGTTT
  601 ATCTATGAGC CGCTATTGCA AGGTGCTGGA GGGATGTTAA TGTATAATCC
  651 CGAAGGCCTA AAGGAGATTC TCAAGCTTGC CAAGCATTAC GGGGTTCTCT
  701 GTATTGCTGA TGAAATTCTT ACTGGCTTTG GCCGTACGGG TCCACTGTTT
  751 GCTTCTGAAT TTACAGACAT TCCTCCTGAC ATTATCTGTC TTTCTAAAGG
  801 TCTTACAGGA GGCTATCTCC CTCTAGCCTT GACAGTAACC ACTAAAGAAA
  851 TTCATGATGC CTTTGTCTCC CAAGATCGGA TGAAGGCACT GCTTCATGGC
  901 CATACCTTCA CAGGAAATCC TTTAGGCTGT AGTGCTGCCC TCGCTTCTTT
  951 GGATCTCACC CTATCTCCAG AATGCCTACA ACAAAGGCAA ATGATAGAAC
1001 GGTGTCATCA AGAGTTCCAA GAAGCTCATG GTTCCCTATG GCAACGGTGT
1051 GAGGTTCTGG GCACGGTACT CGCTCTAGAT TACCCTGCAG AAGCTACAGG
1101 ATATTTTTCA CAATATAGAG ACCATCTCAA TCGCTTTTTC TTAGAACGTG
1151 GAGTCCTTCT TCGTCCTTTA GGGAACACAC TGTATGTGCT GCCCCCCTAC
1201 TGTATCCAAG AAGAAGATCT CCGGATTATT TATTCTCACC TACAGGATGC
1251 CCTATGTCTA CAACCACAGT AA
```

The PSORT algorithm predicts cytoplasm (0.2930).

The protein was expressed in E. coli and purified as a GST-fusion product (FIG. 141A) and also in his-tagged form. The recombinant proteins were used to immunise mice, whose sera were used in a Western blot (FIG. 141B) and for FACS analysis.

These experiments show that cp7374 is a surface-exposed and immunoaccessible protein, and that it is a useful immunogen. These properties are not evident from the sequence alone.

### Example 142

The following C. pneumoniae protein (PID 4377377) was expressed <SEQ ID 283; cp7377>:

```
    1 MREFTVSWSL EDIREIYHTP VFELIHKANA ILRSNFLHSE LQTCYLISIK
  51 TGGCVEDCAY CAQSSRYHTH VTPEPMMKIV DVVERAKRAV ELGATRVCLG
101 AAWRNAKDDR YFDRVLAMVK SITDLGAEVC CALGMLSEEQ AKKLYDAGLY
151 AYNHNLDSSP EFYETIITTR SYEDRLNTLD VVNKSGISTC CGGIVGMGES
201 EEDRIKLLHV LATRDHIPES VPVNLLWPID GTPLQDQPPI SFWEVLRTIA
251 TARVVFPRSM VRLAAGRAFL TVEQQTLCFL AGANSIFYGD KLLTVENNDI
301 DEDAEMIKLL GLIPRPSFGI ERGNPCYANN S*
```

The cp7377 nucleotide sequence <SEQ ID 284> is:

```
    1 ATGCGTGAAG AAACTGTATC CTGGTCATTA GAAGACATCC GCGAAATTTA
  51 TCACACTCCC GTATTTGAGC TGATTCACAA AGCCAATGCC ATATTGCGTA
101 GTAATTTCCT CCATTCAGAA CTGCAGACTT GCTATCTGAT TTCGATTAAA
151 ACTGGTGGAT GCGTTGAAGA TTGCGCCTAC TGTGCCCAAT CTTCCCGCTA
201 TCATACCCAC GTCACACCAG AACCTATGAT GAAAATTGTA GACGTTGTGG
251 AAAGGGCAAA ACGTGCTGTA GAGCTAGGCG CCACTCGTGT GTGTCTTGGG
301 GCTGCCTGGC GCAATGCTAA GGACGATCGA TACTTTGATA GAGTCCTCGC
351 TATGGTGAAA AGTATCACAG ATCTCGGAGC CGAGGTTTGT TGTGCTTTAG
401 GCATGCTCTC CGAAGAGCAA GCTAAAAAAC TGTATGATGC AGGACTTTAT
451 GCCTACAATC ATAATTTAGA CTCTTCTCCG GAATTCTATG AAACTATAAT
501 CACAACACGT TCTTATGAAG ATCGCCTCAA CACTCTTGAT GTAGTAAATA
551 AATCTGGCAT TAGTACATGC TGCGGTGGTA TTGTAGGTAT GGGAGAATCT
601 GAAGAAGACC GTATAAAGCT TCTTCATGTT CTTGCAACAA GAGATCATAT
651 CCCAGAATCC GTACCTGTAA ATTTACTTTG GCCGATTGAC GGCACGCCTT
701 TGCAAGACCA GCCTCCGATT TCTTTCTGGG AAGTCTTGCG AACCATAGCA
751 ACGGCACGGG TTGTTTTCCC CAGATCCATG GTACGACTTG CTGCAGGACG
801 CGCTTTCCTC ACAGTAGAAC AACAAACCTT ATGTTTTCTA GCCGGTGCCA
851 ACTCCATATT CTATGGAGAT AAACTGTTGA CTGTAGAAAA CAATGATATA
901 GATGAAGATG CTGAAATGAT CAAACTTTTA GGCTTAATCC CTCGCCCTTC
951 ATTTGGAATA GAAAGAGGTA ACCCATGTTA TGCCAACAAT TCCTAA
```

The PSORT algorithm predicts cytoplasm (0.2926).

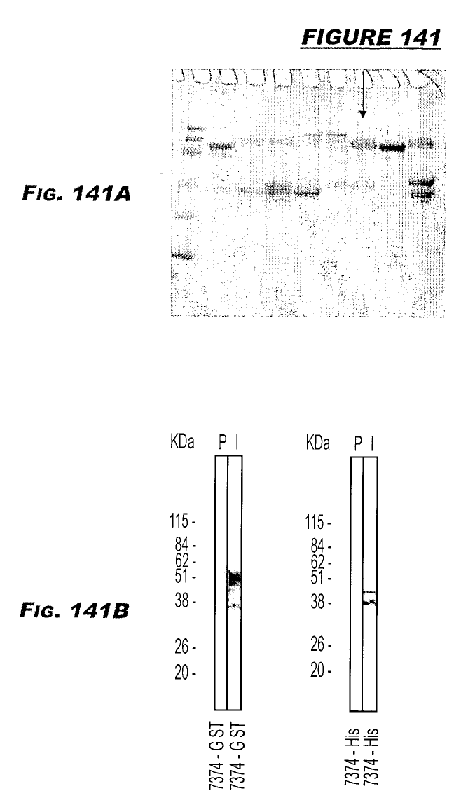

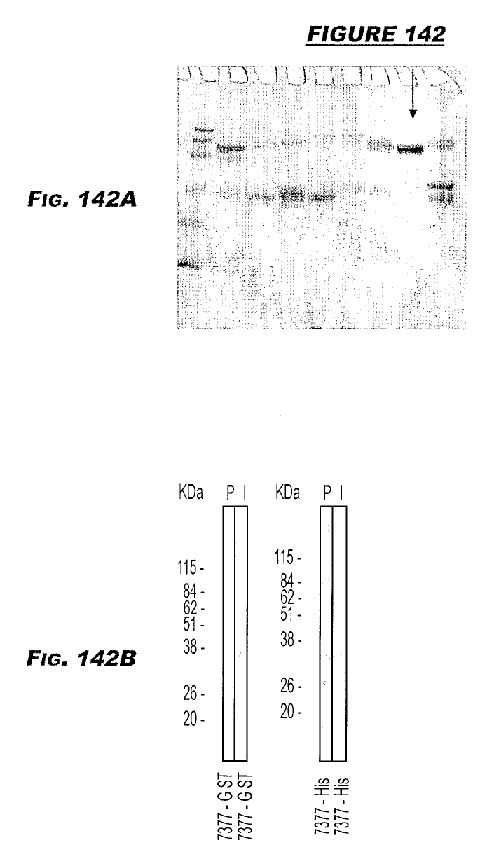

The protein was expressed in E. coli and purified as a GST-fusion product (FIG. 142A) and also in his-tagged form. The recombinant proteins were used to immunise mice, whose sera were used in a Western blot (FIG. 142B) and for FACS analysis.

These experiments show that cp7377 is a surface-exposed and immunoaccessible protein, and that it is a useful immunogen. These properties are not evident from the sequence alone.

### Example 143

The following C. pneumoniae protein (PID 4377407) was expressed <SEQ ID 285; cp7407>:

```
    1 MVCPNNSWFR MCGNFNCEWV EVTTTEETTR QSASDISEEA GSSGGAAPIT
  51 TQPTKITKVE KRVQFNTAQG DEFTIHMIQE AGELVDSILS HRRTQGCTFY
101 CYDSYATGCG QRCGSFGRLI CGTYKACCLD REDNQVAGLV HECEQTHGPI
151 AVALAAKTMG LNLMELVEKN TILSEEQKNE FRQHCSEAKT QLYGTMQSLS
201 QNFFLEGVNS IRERFLDDSL VQAVLSFIAT RSWEKTIESE EASGTSSASN
251 STRIPACYIL NTSPITTSRL SCGSRDARRP SSVGAEPQYV AKKYNDNGMA
301 RQLGKIQVTN LKTGDFSALG PRGLLIVKML NSFLLSASQS TSSILKHTGG
351 EICYTCPNFR DIVVLLMLAI GYCPANTDET SVVDIHMIDD PIMTIFYRLQ
401 YSYRTGKTSA SFLKKKPSLV RQESLDCPTP AESVPLMSSL EEEDENEDDD
451 EDGNLAYQQR ILECSGHLQT LFLGIKINKE *
```

The cp7407 nucleotide sequence <SEQ ID 286> is:

```
      1 ATGGTTTGCC CAAATAATTC TTGGTTCAGA ATGTGTGGAA ATTTCAACTG
    51 CGAATGGGTT GAAGTAACAA CAACAGAAGA AACAACGCGG CAATCGGCTT
  101 CAGATATAAG CGAAGAAGCT GGTTCGAGTG GAGGAGCTGC TCCTATAACT
  151 ACGCAACCTA CTAAAATTAC AAAAGTAGAG AAACGTGTCC AATTTAATAC
  201 TGCTCAAGGT GATGAAAGTA CAATACACAT GATCCAAGAA GCAGGAGAAT
  251 TGGTAGACTC CATTCTATCA CATAGACGAA CGCAAGGATG TACAGAGTAT
  301 TGTTATGACA GTTACGCAAC TGGATGTGGT CAGCGTTGCG GATCTTTTGG
  351 AAGACTCATT TGTGGAACGT ATAAAGCGTG TTGCTTAGAC AGAGAGGATA
  401 ATCAGGTTGC TGGACTTGTC CATGAATGCG AACAGACCCA TGGTCCTATT
  451 GCCGTTGCTT TAGCTGCTAA AACTATGGGC CTCAACTTAA TGGAACTTGT
  501 AGAAAAAAAC ACTATTTTGT CTGAAGAACA GAAAAATGAA TTTAGACAGC
  551 ATTGCTCGGA AGCTAAAACC CAACTCTATG GAACGATGCA GAGCCTTTCT
  601 CAAAACTTTT TCCTTGAAGG AGTCAACAGC ATTAGAGAAC GCGGTCTAGA
  651 CGATTCACTA GTCCAAGCCG TGCTAAGCTT TATTGCTACA AGGTCTTGGG
  701 AAAAAACTAT AGAATCAGAG GAAGCCTCAG GAACATCTTC TGCTTCTAAT
  751 TCTACACGCA TTCCTGCGTG CTATATCTTA AATACGAGCC CCTTAACGAC
  801 GTCACGCCTA TCCTGTGGAT CAAGAGATGC GCGACGCCCA TCTTCAGTCG
  851 GTGCAGAGCC CCAGTACGTA GCAAAAAAAT ACAATGACAA TGGCATGGCC
  901 AGACAATTAG GAAAAATCCA AGTCACCAAT CTAAAAACAG GAGATTTTTC
  951 AGCTTTAGGT CCTTTTGGTC TCCTGATTGT GAAAATGCTG AATAGCTTTC
1001 TCTTATCTGC ATCACAAAGC ACATCTTCTA TTCTAAAGCA CACAGGTGGA
1051 GAAATATGTT ATACGTGCCC AAATTTTCGT GATATCGTCG TTTTATTGAT
1101 GTTAGCGATT GGCTATTGCC CTGCAAATAC CGATGAGACA TCTGTCGTAG
1151 ATATACACAT GATAGATGAT CCGATTATGA CCATCTTCTA TCGACTACAA
1201 TACAGCTATA GAACAGGGAA AACTTCAGCA TCGTTTTTAA AAAAGAAACC
1251 CTCATTAGTA AGACAGGAAA GTCTTGATTG TCCTACCCCT GCAGAATCTG
1301 TCCCTCTCAT GTCAAGTCTC GAAGAAGAAG ATGAAAATGA AGATGATGAT
1351 GAGGATGGGA ATTTGGCGTA TCAACAGCGT ATCCTTGAAT GCTCGGGTCA
1401 TTTACAAACT CTATTTTTAG GGATAAAAAT AAACAAAGAA TAA
```

The PSORT algorithm predicts inner membrane (0.1319).

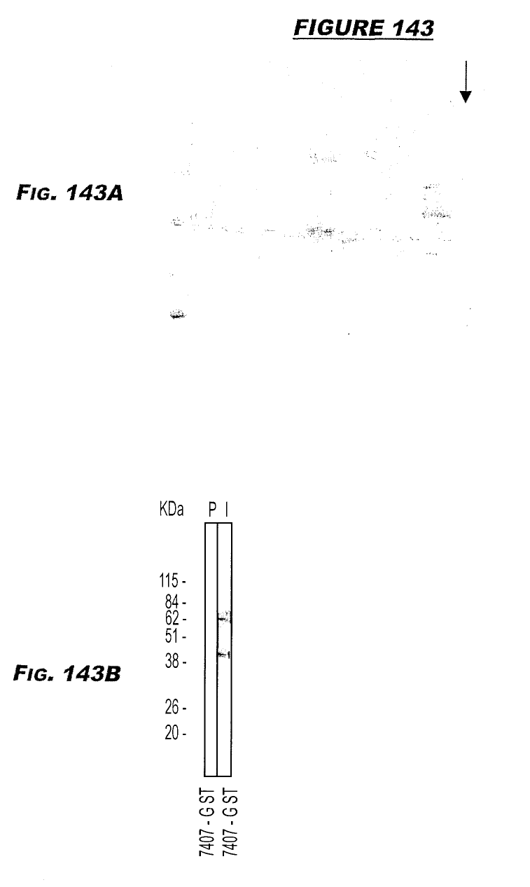

The protein was expressed in E. coli and purified as a GST-fusion product (FIG. 143A). The recombinant protein was used to immunise mice, whose sera were used in a Western blot (FIG. 143B) and for FACS analysis.

These experiments show that cp7407 is a surface-exposed and immunoaccessible protein, and that it is a useful immunogen. These properties are not evident from the sequence alone.

### Example 144

The following C. pneumoniae protein (PID 4376432) was expressed <SEQ ID 287; cp6432>:

```
    1 MTRSTIESSD SLCSRSFSQK LSVQTLKNLC ESRLMKITSL VIAFLTLIVG
  51 GALIALAGGG VLSFPLGLIL GSVLVLFSSI YLVSCCKFFT LKEMTMTCSV
101 KSKINIWFEK QRNKDIEKAL ENPDLFGENK RNVGNRSARN QLEMILHETD
151 GIILKRYMKG AKMYFYL*
```

The cp6432 nucleotide sequence <SEQ ID 288> is:

```
    1 ATGACTAGAA GTACTATTGA AAGCAGTGAT TCGCTATGCT CAAGGTCTTT
  51 TTCTCAAAAA TTAAGTGTCC AGACATTAAA AAATCTCTGT GAAAGTAGAT
101 TAATGAAGAT CACTTCTCTT GTGATTGCTT TCCTAACTCT AATTGTGGGG
151 GGTGCTCTTA TAGCTTTAGC AGGAGGGGGG GTTCTTTCTT TCCCTCTTGG
201 GCTAATCTTA GGAAGCGTAC TCGTTTTGTT TTCTTCTATC TATTTAGTCT
251 CTTGTTGTAA ATTTTTCACT TTAAAAGAGA TGACAATGAC CTGTAGTGTC
301 AAATCTAAAA TCAATATATG GTTTGAAAAG CAACGAAACA AAGACATCGA
351 AAAGGCATTA GAGAATCCAG ATCTCTTTGG AGAAAATAAG AGAAATGTTG
401 GAAATCGTTC GGCAAGAAAT CAACTAGAAA TGATCTTACA CGAGACTGAC
451 GGAATTATTT TGAAAAGATA TATGAAAGGA GCTAAAATGT ACTTTTATTT
501 ATGA
```

The PSORT algorithm predicts inner membrane (0.5394).

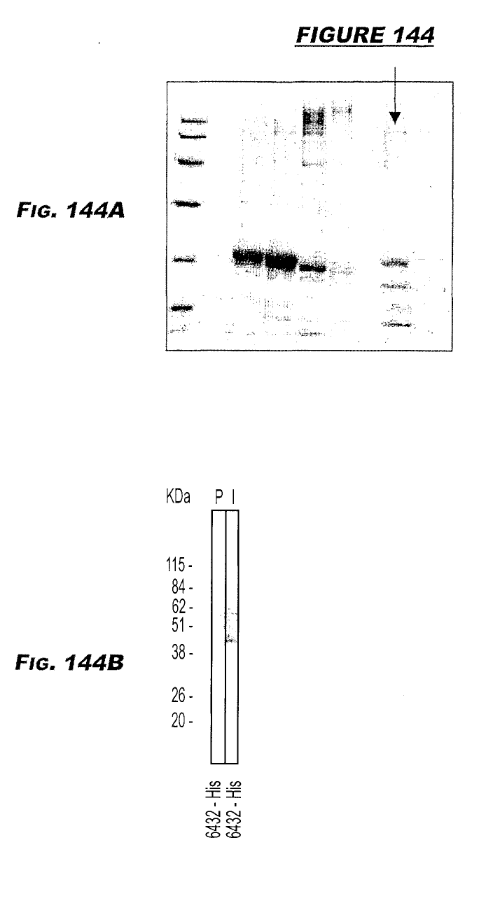

The protein was expressed in E. coli and purified as a his-tagged product (FIG. 144A). The recombinant protein was used to immunise mice, whose sera were used in a Western blot (FIG. 144B) and for FACS analysis.

These experiments show that cp6432 is a surface-exposed and immunoaccessible protein, and that it is a useful immunogen. These properties are not evident from the sequence alone.

### Example 145

The following C. pneumoniae protein (PID 4376433) was expressed <SEQ ID 289; cp6433>:

```
    1 MNWVPKTIDH VDPESEIDIR KVVSCYKLIK ECQPEFRSLI SELLGVIRCG
  51 LRLLKRSKYQ EQARTVSDED APLFCLTRSY YQDGYLTPLR AGPRDLINHY
101 IHLRRRENPK HFFSPKHPCY YARLAFNESV CVYRELFDIE RLTKMYVEGD
151 YSKEQEKNLQ AILSFVKTLD EGKDFLIEHK DTDLIGRGFT DVFCT*
```

The cp6433 nucleotide sequence <SEQ ID 290> is:

```
    1 ATGAATTGGG TTCCAAAAAC AATAGACCAT GTAGATCCAG AATCAGAGAT
  51 AGATATACGT AAAGTCGTCT CCTGCTATAA GTTGATAAAA GAATGTCAAC
101 CTGAATTTCG ATCTCTTATA AGTGAATTAC TAGGAGTGAT TCGGTGTGGC
151 TTAAGACTAT TAAAACGTTC TAAGTATCAA GAACAGGCTA GAACTGTATC
201 TGATGAAGAT GCACCTCTTT TCTGCCTGAC TCGTTCTTAT TATCAAGATG
251 GTTATCTCAC GCCATTAAGA GCAGGACCTC GTGATCTTAT AAATCACTAT
301 ATACACTTGC GTCGCCGAGA GAATCCTAAG CATTTTTTCA GTCCTAAGCA
351 TCCATGTTAT TATGCTCGAT TGGCTTTTAA TGAGTCAGTG TGTGTCTATA
401 GAGAACTCTT TGATATAGAG CGACTTACAA AAATGTATGT CGAGGGTGAT
451 TATTCTAAAG AACAAGAGAA AAACCTACAG GCTATTCTTA GTTTTGTGAA
501 AACTCTAGAT GAAGGAAAGG ACTTTCTTAT TGAACATAAA GATACCGATC
551 TCATTGGGAG AGGTTTTACT GATGTGTTCT GCACTTAA
```

The PSORT algorithm predicts cytoplasm (0.4068).

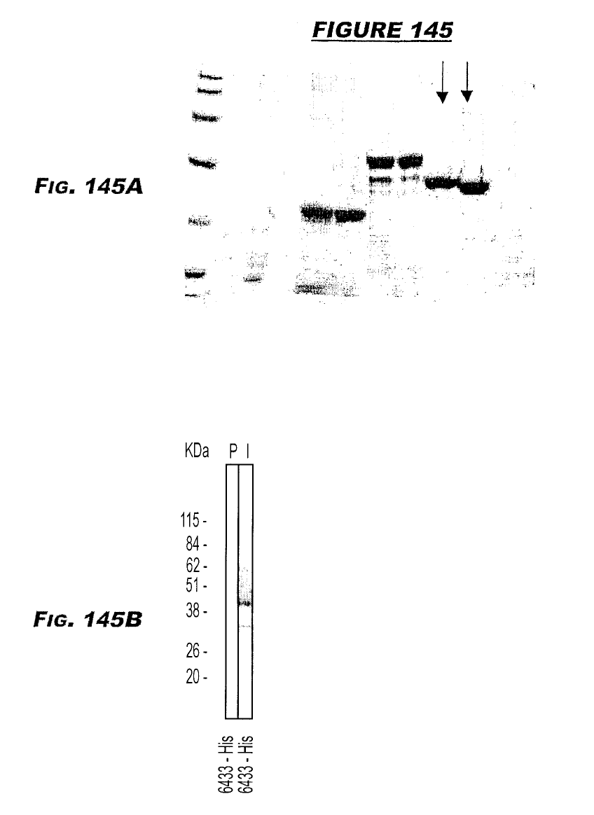

The protein was expressed in E. coli and purified as a his-tagged product (FIG. 145A). The recombinant protein was used to immunise mice, whose sera were used in a Western blot (FIG. 145B) and for FACS analysis.

These experiments show that cp6433 is a surface-exposed and immunoaccessible protein, and that it is a useful immunogen. These properties are not evident from the sequence alone.

### Example 146

The following C. pneumoniae protein (PID 4376643) was expressed <SEQ ID 291; cp6643>:

```
    1 MGYLPVSATD VLFESPAAPL INSANTQNQK LIELKGKQQA ESSPRTITSV
  51 ILEVLLVIGC CLIVLSLLAI RPALQFTLET GHPAAIAVLA VSGTILLVAV
101 IILFCFLAAV PFAAKKTYKY VKTVDDYASW HSHQQTPTLG TIFSGIVYAE
151 SQAQL*
```

The cp6643 nucleotide sequence <SEQ ID 292> is:

```
    1 ATGGGATATC TTCCAGTATC TGCTACGGAC GTTCTTTTTG AAAGTCCAGC
  51 CGCTCCCTTA ATCAATAGCG CAAACACACA AAATCAGAAA CTCATAGAAC
101 TCAAGGGGAA GCAGCAAGCT GAGTCTTCTC CACGGACAAT CACTTCTGTC
151 ATATTGGAAG TTCTCCTAGT GATCGGATGC TGCCTCATAG TTCTTAGTTT
201 ATTGGCAATC CGCCCTGCTC TGCAATTCAC TCTAGAAACT GGACATCCAG
251 CTGCCATTGC AGTCCTTGCT GTCTCAGGAA CAATTCTATT GGTGGCTGTT
301 ATCATCTTGT TTTGCTTTCT AGCAGCTGTG CCATTCGCTG CTAAGAAAAC
351 TTATAAATAT GTTAAGACGG TTGATGACTA TGCTTCTTGG CATTCTCATC
401 AGCAAACACC GACCCTAGGC ACTATCTTTT CAGGTATCGT CTATGCAGAA
451 TCCCAGGCGC AATTATAG
```

The PSORT algorithm predicts inner membrane (0.6859).

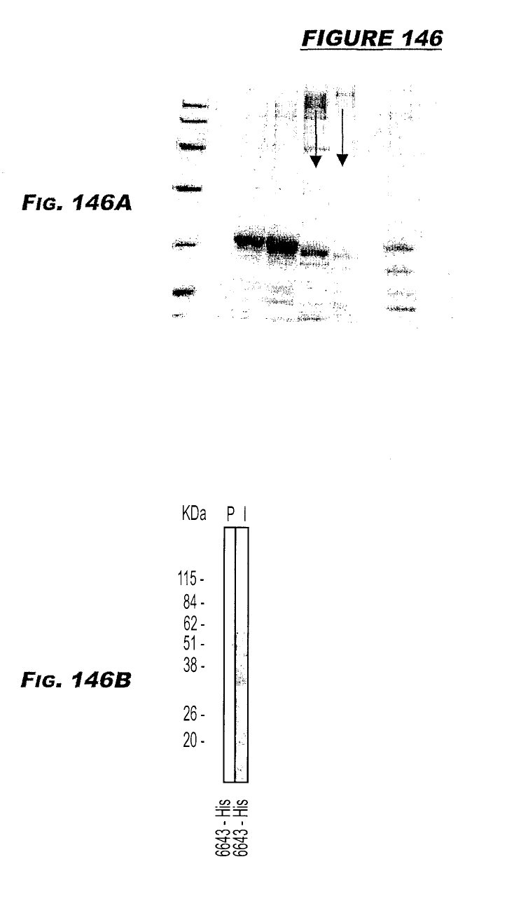

The protein was expressed in E. coli and purified as a his-tagged product (FIG. 146A). The recombinant protein was used to immunise mice, whose sera were used in a Western blot (FIG. 146B) and for FACS analysis.

These experiments show that cp6643 is a surface-exposed and immunoaccessible protein, and that it is a useful immunogen. These properties are not evident from the sequence alone.

### Example 147

The following C. pneumoniae protein (PID 4376722) was expressed <SEQ ID 293; cp6722>:

```
    1 VSSTLNGVFP SSLPEESADL FITNKEIVAL GEKGNVFLTH SIPMHIAAIT
  51 ILVIVALAGI AIICLGCYSQ SILLIAVGIV LTILTLLCLQ ALVGFIFKIR
101 QLPQQLHTTV QFIREKIRPE SSLQLVTNAQ RKTTQDTLKL YEELCDLSQK
151 EFKLQSTLYQ KRFELSHKNE KTNQN*
```

The cp6722 nucleotide sequence <SEQ ID 294> is:

```
    1 GTGTCTAGTA CTTTAAACGG GGTATTTCCC TCATCCCTTC CGGAAGAGTC
  51 TGCTGATTTA TTCATTACGA ATAAGGAGAT CGTAGCTTTG GGGGAGAAGG
101 GCAATGTTTT TCTCACCCAC TCCATTCCTA TGCATATTGC TGCGATTACG
151 ATCTTAGTGA TTGTAGCTCT TGCTGGAATC GCTATTATCT GTTTGGGTTG
201 CTATAGCCAA AGCATTCTGT TGATTGCCGT TGGCATTGTT CTTACTATTT
251 TGACTCTTCT CTGCCTACAA GCCTTGGTAG GATTTATTAA ATTCATCCGG
301 CAGCTCCCTC AGCAGCTCCA TACGACAGTA CAATTTATCA GGGAGAAGAT
351 TCGACCTGAA TCCTCTCTAC AGCTTGTAAC CAATGCACAG AGAAAAACCA
401 CTCAAGATAC GCTAAAGTTA TACGAAGAAC TCTGCGACCT CTCACAAAAA
451 GAGTTCAAAC TGCAATCAAC TCTTTATCAA AAACGTTTTG AGCTTTCTCA
501 CAAGAATGAA AAGACAAATC AAAACTAG
```

The PSORT algorithm predicts inner membrane (0.6668).

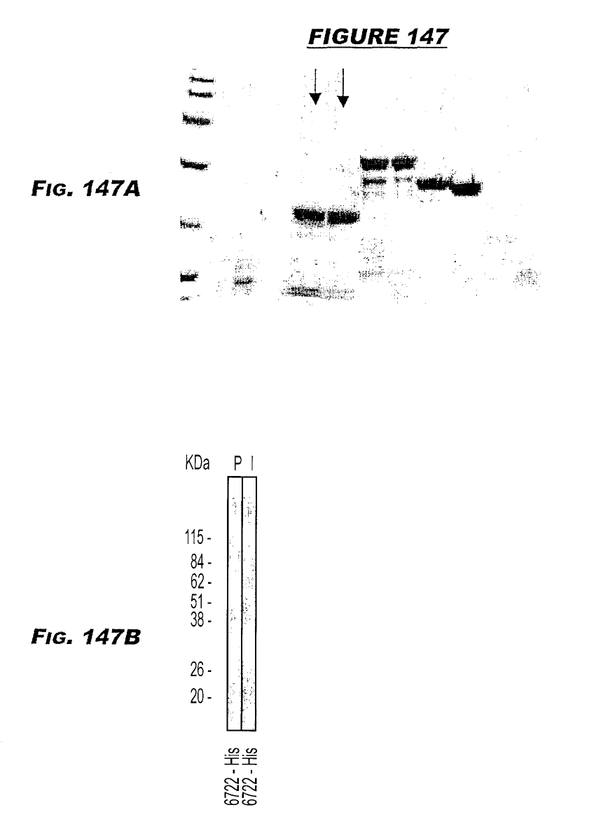

The protein was expressed in E. coli and purified as a his-tagged product (FIG. 147A). The recombinant protein was used to immunise mice, whose sera were used in a Western blot (FIG. 147B) and for FACS analysis.

These experiments show that cp6722 is a surface-exposed and immunoaccessible protein, and that it is a useful immunogen. These properties are not evident from the sequence alone.

### Example 148

The following C. pneumoniae protein (PID 4377253) was expressed <SEQ ID 295; cp7253>:

```
    1 MSELAPCSTG LQMVPHTQVH HALDTRRVIL TIAACLSLIA GIVLVGLGAA
  51 AILPSLFGVI GGMILILFSS IALIYLYKKT REVDQIALEP LPEMISKDQS
101 IIDFVKTRDY ASLEKKATFA YTHTHYYDGS MVFYREIPRF MLGSYLALRK
151 DMDRQALF*
```

The cp7253 nucleotide sequence <SEQ ID 296> is:

```
    1 ATGAGCGAGC TCGCCCCCTG CTCGACAGGA TTGCAGATGG TCCCCCATAC
  51 GCAGGTCCAT CATGCCCTTG ATACGCGGAG AGTCATTCTA ACGATAGCCG
101 CCTGTCTGTC TTTAATTGCA GGAATCGTGT TGGTTGGCTT AGGTGCTGCA
151 GCAATCCTGC CCTCGCTTTT TGGAGTCATT GGAGGAATGA TTCTTATTCT
201 GTTTTCTTCG ATCGCCCTCA TTTATTTATA CAAGAAGACA AGGGAGGTGG
251 ATCAGATTGC TCTGGAGCCT CTTCCTGAGA TGATTTCTAA AGATCAAAGC
301 ATTATAGATT TTGTAAAGAC ACGAGACTAT GCATCTTTAG AAAAGAAAGC
351 GACCTTTGCT TATACTCATA CTCATTATTA CGATGGAAGC ATGGTCTTCT
401 ATAGGGAGAT CCCTAGATTT ATGTTAGGCT CTTATCTCGC GCTTCGCAAA
451 GACATGGACC GCCAAGCTCT TTTTTGA
```

The PSORT algorithm predicts inner membrane (0.5394).

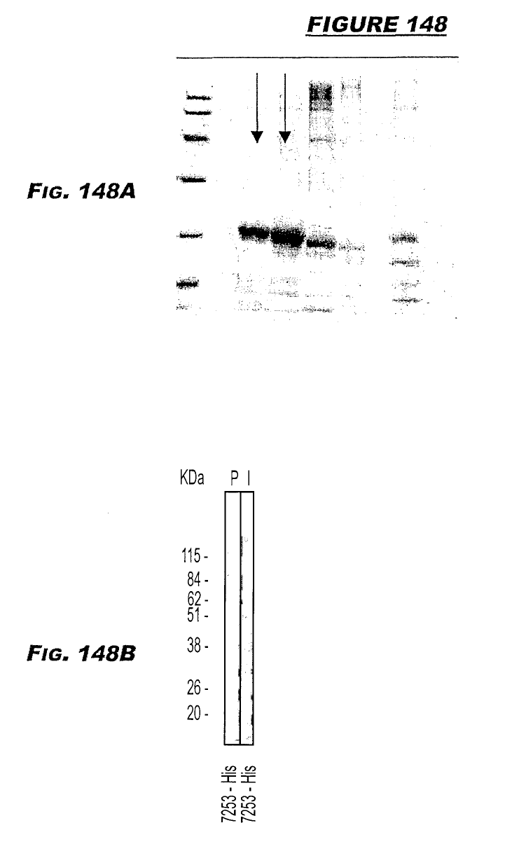

The protein was expressed in E. coli and purified as a his-tagged product (FIG. 148A). The recombinant protein was used to immunise mice, whose sera were used in a Western blot (FIG. 148B) and for FACS analysis.

These experiments show that cp7253 is a surface-exposed and immunoaccessible protein, and that it is a useful immunogen. These properties are not evident from the sequence alone.

### Example 149

The following C. pneumoniae protein (PID 4376264) was expressed <SEQ ID 297; cp6264>:

```
    1 VISGLLFLLV RREVPTVRSE EIPRGVSVTP SEEPALEKAQ KEPETKKILD
  51 RLPKELDQLD TYIQEVFACL ERLKDPKYED RGLLTEAKEK LRVFDVVEKD
101 MMSEFLDIQR VLNEEAYYVE HCQDPLENIA YEIFSSQELR DYYCAGVCGY
151 LPSGDARADR LKRSVKEVMD RFMRVIWKSW EASVMLDESY GVARELFKKA
201 VGVLEESVYK ILFKSYRDAF YECEKAKIQR DGRFKWL*
```

The cp6264 nucleotide sequence <SEQ ID 298> is:

```
    1 GTGATTTCGG GACTTCTATT CCTTCTAGTA AGACGAGAGG TTCCGACAGT
  51 ACGTTCAGAG GAAATTCCCA GAGGGGTTTC TGTGACCCCT TCTGAAGAGC
101 CTGCTCTAGA GAAGGCTCAA AAAGAACCGG AGACAAAGAA AATTTTAGAT
151 CGGTTGCCGA AGGAATTGGA TCAGTTAGAT ACGTATATTC AGGAAGTGTT
201 TGCATGTTTA GAGAGGCTGA AGGATCCTAA GTACGAAGAT CGAGGTCTTT
251 TAACAGAGGC GAAGGAGAAA CTTCGAGTTT TTGACGTTGT TGAGAAAGAT
301 ATGATGTCAG AGTTTTTAGA CATACAACGA GTGTTGAATG AGGAAGCATA
351 TTATGTAGAA CATTGTCAAG ATCCCCTAGA GAATATAGCC TACGAGATTT
401 TCTCTTCCCA AGAGCTTCGT GATTACTACT GTGCAGGGGT GTGTGGGTAT
451 TTGCCTTCTG GGGATGCTCG AGCGGATCGA TTAAAGAGAT CAGTTAAGGA
501 GGTAATGGAT CGCTTTATGA GGGTGACCTG GAAATCTTGG GAGGCATCAG
551 TCATGTTGGA TCATAGCTAT GGGGTAGCGC GAGAGTTATT CAAGAAGGCA
601 GTAGGAGTAC TAGAGGAGAG TGTCTATAAA ATTCTGTTTA AGAGCTATAG
651 AGATGCGTTT TATGAATGTG AGAAGGCAAA GATCCAGAGG GATGGGCGTT
701 TCAAATGGTT ATAG
```

The PSORT algorithm predicts cytoplasm (0.2817).

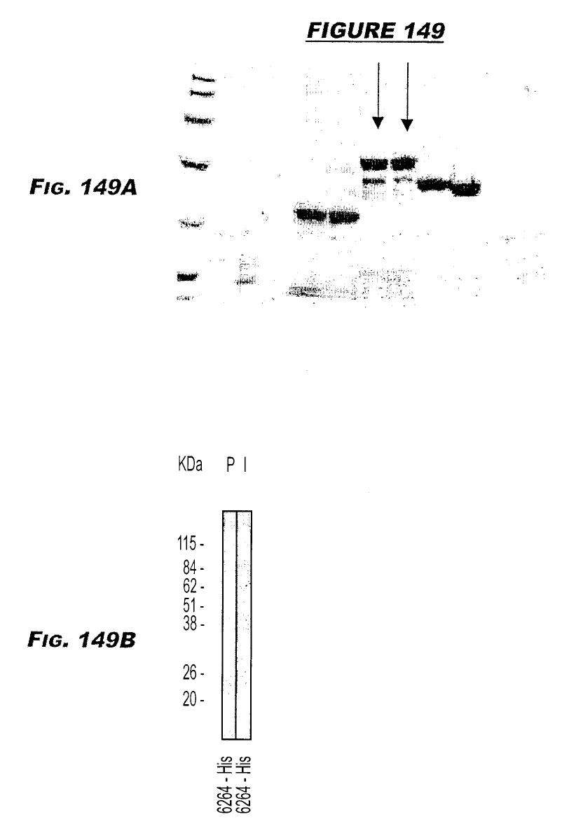

The protein was expressed in E. coli and purified as a his-tagged product (FIG. 149A). The recombinant protein was used to immunise mice, whose sera were used in a Western blot (FIG. 149B) and for FACS analysis.

These experiments show that cp6264 is a surface-exposed and immunoaccessible protein, and that it is a useful immunogen. These properties are not evident from the sequence alone.

### Example 150

The following C. pneumoniae protein (PID 4376266) was expressed <SEQ ID 299; cp6266>:

```
    1 MLLLISGALF LTLGIPGLSA AISFGLGIGL SALGGVLMIS GLLCLLVKRE
  51 IPTVRPEEIP EGVSLAPSEE PALQAAQKTL AQLPKELDQL DTDIQEVFAC
101 LRKLKDSKYE SRSFINDAKK ELRVFDFVVE DTLSEIFELR QIVAQEGWDL
151 NFLINGGRSL MMTAESESLD LFHVSKRLGY LPSGDVRGEG LKKSAKEIVA
201 RLMSLHCEIH KVAVAFDRNS YAMAEKAFAK ALGALEESVY RSLTQSYRDK
251 FLESERAKIP WNGHITWLRD DAKSGCAEKK LGMPRNVGRN LGKQSFG*
```

The cp6266 nucleotide sequence <SEQ ID 300> is:

```
    1 ATGCTCTTAC TGATTTCAGG AGCTCTCTTT CTGACGTTAG GGATTCCAGG
  51 ATTGAGTGCA GCAATTTCTT TTGGATTAGG CATCGGTCTC TCCGCATTAG
101 GAGGAGTGCT GATGATTTCG GGACTACTAT GTCTTTTAGT AAAACGAGAG
151 ATTCCGACAG TACGACCAGA AGAAATTCCT GAAGGGGTTT CGCTGGCTCC
201 TTCTGAGGAG CCAGCTCTAC AGGCAGCTCA GAAGACTTTA GCTCAGCTGC
251 CTAAGGAATT GGATCAGTTA GATACAGATA TTCAGGAAGT GTTCGCATGT
301 TTAAGAAAGC TGAAAGATTC TAAGTATGAA AGTCGAAGTT TTTTAAACGA
351 TGCTAAGAAG GAGCTTCGAG TTTTTGACTT TGTGGTTGAG GATACCCTCT
401 CGGAGATTTT CGAGTTGCGG CAGATTGTGG CTCAAGAGGG ATGGGATTTA
451 AACTTTTTGA TCAATGGGGG ACGAAGCCTC ATGATGACTG CAGAATCTGA
501 ATCGCTTGAT TTGTTTCATG TATCGAAGCG GCTAGGGTAT TTACCTTCTG
551 GGGATGTTCG AGGGGAGGGG TTAAAGAAAT CTGCGAAGGA GATAGTCGCT
601 CGTTTGATGA GCTTGCATTG CGAGATTCAC AAGGTGGCGG TAGCGTTTGA
651 TAGGAATTCC TATGCGATGG CAGAAAAGGC GTTTGCGAAA GCGTTGGGAG
701 CTTTAGAAGA GAGTGTGTAT CGGAGTCTGA CGCAGAGTTA TAGAGATAAA
751 TTTTTGGAGA GCGAGAGGGC GAAGATCCCA TGGAATGGGC ATATAACCTG
801 GTTAAGAGAT GATGCGAAGA GTGGGTGTGC TGAAAAGAAG CTCGGGATGC
851 CGAGGAACGT TGGAAGAAAT TTAGGAAAGC AGTCTTTTGG GTAG
```

The PSORT algorithm predicts inner membrane (0.3590).

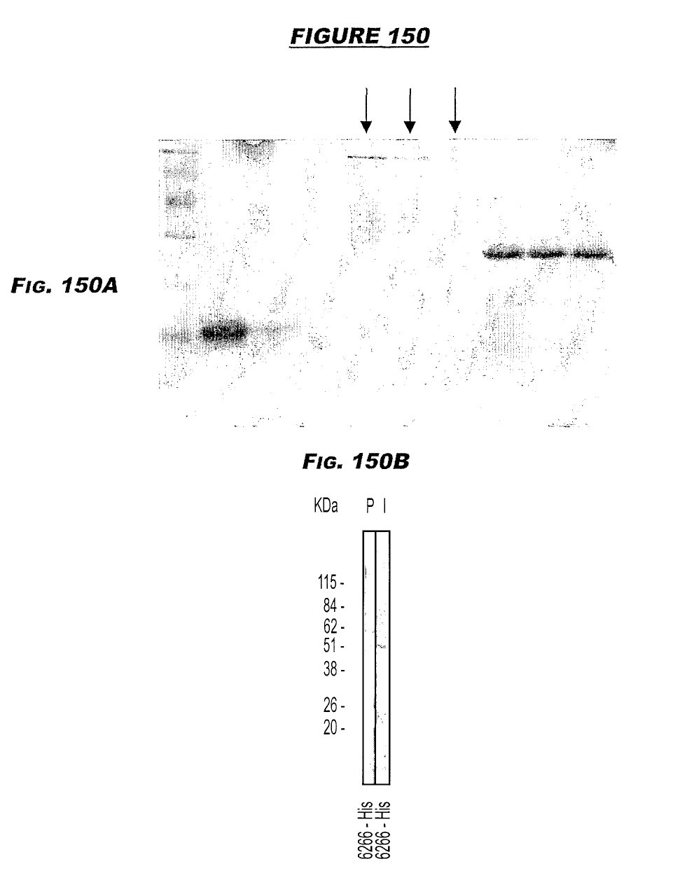

The protein was expressed in E. coli and purified as a his-tag product (FIG. 150A). The recombinant protein was used to immunise mice, whose sera were used in a Western blot (FIG. 150) and for FACS analysis.

These experiments show that cp6266 is a surface-exposed and immunoaccessible protein and that they it is a useful immunogen. These properties are not evident from the sequence alone.

### Example 151

The following C. pneumoniae protein (PID 4376895) was expressed <SEQ ID 301; cp6895>:

```
    1 MKIKKSFQYS LCQAKRFQNM LPNHFDPCLQ PVNLQLKQDR LAYGELIILL
  51 SKYQQKTFSS LLKEETCSLN RAKQHLLYKI LRDFNTMQHL RSLGLNGWGE
101 IPMSPCL*
```

The cp6895 nucleotide sequence <SEQ ID 302> is:

```
    1 ATGAAGATTA AAAAATCTTT TCAATACAGT TTATGCCAAG CAAAGAGATT
  51 TCAGAACATG CTGCCAAACC ACTTTGATCC ATGTTTGCAG CCAGTGAATT
101 TACAACTCAA ACAAGACAGA TTGGCATACG GGGAGCTCAT CATATTGCTA
151 TCTAAATATC AACAAAAGAC CTTTTCCTCT TTGTTGAAGG AAGAAACATG
201 TTCTCTTAAT CGTGCGAAGC AGCACTTATT GTATAAGATT TTGAGAGATT
251 TTAATACTAT GCAGCATCTA AGGTCCCTCG GATTAAATGG TTGGGGAGAG
301 ATCCCTATGA GTCCTTGCCT CTAA
```

The PSORT algorithm predicts cytoplasm (0.3264).

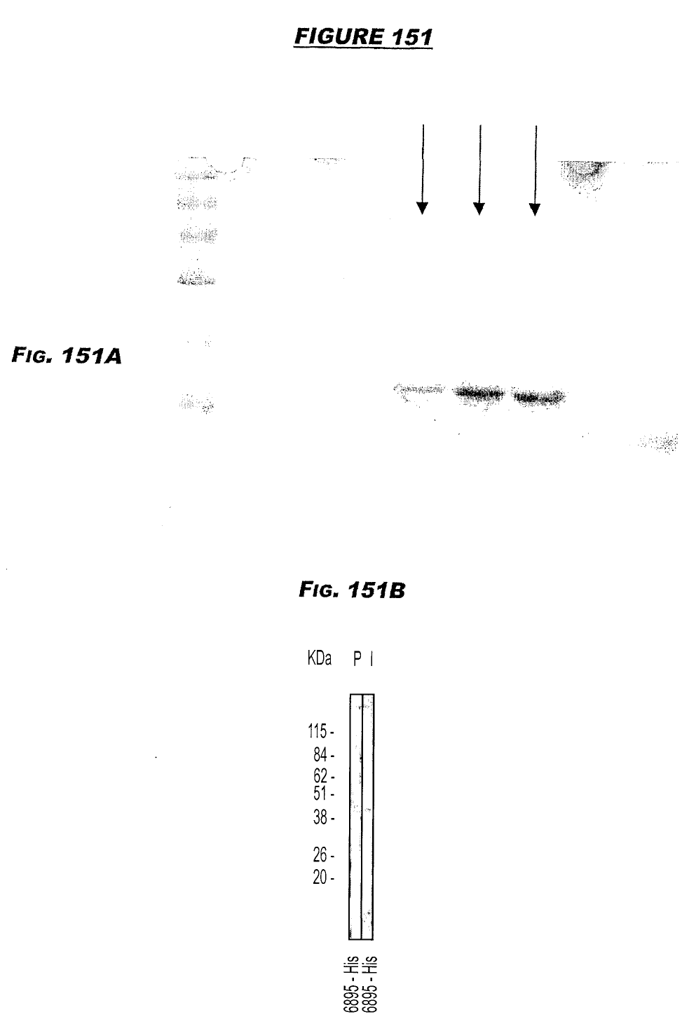

The protein was expressed in E. coli and purified as a his-tag product (FIG. 151A). The recombinant protein was used to immunise mice, whose sera were used in a Western blot (FIG. 151B) and for FACS analysis.

These experiments show that cp6895 is a surface-exposed and immunoaccessible protein and that it is a useful immunogen. These properties are not evident from the sequence alone.

### Example 152 and Example 153

The following C. pneumoniae protein (PID 4376282) was expressed <SEQ ID 303; cp6282>:

```
    1 MSLLNLPSSQ DSASEDSTSQ SQIFDPIRNR ELVSTPEEKV RQRLLSFLMH
  51 KLNYPKKLII IEKELKTLFP LLMRKGTLIP KRRPDILIIT PPTYTDAQGN
101 THNLGDPKPL LLTECKALAV NQNALKQLLS YNYSIGATCI AMAGKHSQVS
151 ALFNPKTQTL DFYPGLPEYS QLLNYFISLN L*
```

The cp6282 nucleotide sequence <SEQ ID 304> is:

```
    1 ATGTCCTTAT TGAACCTTCC CTCAAGCCAG GATTCTGCAT CTGAGGACTC
  51 CACATCGCAA TCTCAAATCT TCGATCCCAT TAGAAATCGG GAGTTAGTTT
101 CTACTCCCGA AGAAAAAGTC CGCCAAAGGT TGCTCTCCTT CCTAATGCAT
151 AAGCTGAACT ACCCTAAGAA ACTCATCATC ATAGAAAAAG AACTCAAAAC
201 TCTTTTTCCT CTGCTTATGC GTAAAGGAAC CCTAATCCCA AAACGCCGCC
251 CAGATATTCT CATCATCACT CCCCCCACAT ACACAGACGC ACAGGGAAAC
301 ACTCACAACC TAGGCGACCC AAAACCCCTG CTACTTATCG AATGTAAGGC
351 CTTAGCCGTA AACCAAAATG CACTCAAACA ACTCCTTAGC TATAACTACT
401 CTATCGGAGC CACCTGCATT GCTATGGCAG GGAAACACTC TCAAGTGTCA
451 GCTCTCTTCA ATCCAAAAAC ACAAACTCTT GATTTTTATC CTGGCCTCCC
501 AGAGTATTCC CAACTCCTAA ACTACTTTAT TTCTTTAAAC TTATAG
```

The PSORT algorithm predicts cytoplasm (0.362).

The following C. pneumoniae protein (PID 4377373) was also expressed <SEQ ID 305; cp7373>:

```
    1 MSTTTVKHFI HTASRWEPVL KEIVASNYWH AQWINTLSFL ENSGAKKISA
  51 SEHPTEVKEE VLKHAAEEFR HGHYLKTQIS RISETSLPDY TSKNLLGGLL
101 TKYYLHLIDL RTCRVLENEY SLSGQTLKTA AYILVTYAIE LRASFLYPLY
151 HDILKEAQSK ITVKSIILEE QGHLQEMERE LKDLPHGEEL LGYACQFEGE
201 LCLQFVERLE QMIFDPSSTF TKF*
```

The cp7373 nucleotide sequence <SEQ ID 306> is:

```
    1 ATGTCTACAA CCACAGTAAA ACACTTTATC CACACAGCCT CTCGTTGGGA
  51 GCCCGTTCTC AAAGAGATCG TAGCTTCCAA CTATTGGCAT GCACAATGGA
101 TAAATACCCT GTCCTTTTTA GAAAATAGTG GAGCAAAAAA AATCTCCGCA
151 AGTGAACATC CTACGGAGGT AAAGGAAGAA GTTTTAAAAC ATGCTGCTGA
201 AGAATTTCGT CATGGTCACT ATCTAAAAAC TCAGATTTCT AGAATCTCAG
251 AGACTTCTCT CCCTGACTAT ACATCTAAAA ATCTTCTGGG AGGCTTACTT
301 ACAAAATATT ACCTCCATCT TCTAGATTTA AGGACGTGCC GAGTACTGGA
351 AAATGAATAC TCCCTATCGG GACAAACGTT AAAAACTGCA GCGTATATTT
401 TAGTTACCTA CGCAATCGAA CTTCGTGCTT CTGAACTTTA TCCTCTGTAT
451 CACGATATTC TGAAAGAAGC TCAAAGTAAA ATAACGGTAA AATCCATTAT
501 CTTAGAAGAG CAAGGCCATC TGCAAGAGAT GGAACGTGAA CTTAAAGATC
551 TCCCCCACGG GGAGGAACTC TTAGGCTATG CTTGCCAATT CGAAGGGGAG
601 CTTTGCTTGC AGTTTGTAGA GAGATTAGAA CAAATGATCT TCGATCCTTC
651 CTCGACTTTT ACAAAGTTCT AG
```

The PSORT algorithm predicts cytoplasm (0.1069).

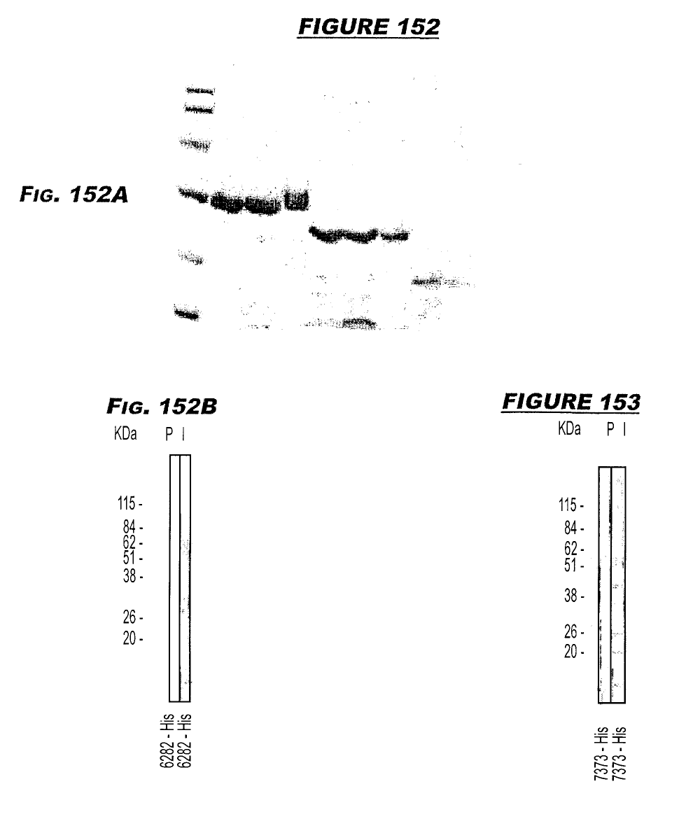

The proteins were expressed in E. coli and purified as his-tag products (FIG. 152A; 6282=lanes 8 & 9; 7373=lanes 2-4). The recombinant proteins were used to immunise mice, whose sera were used in Western blots (FIGS. 152B & 153) and for FACS analysis.

These experiments show that cp6282 & cp7373 are surface-exposed and immunoaccessible proteins and that they are useful immunogens. These properties are not evident from the sequence alone.

### Example 154, Example 155, Example 156, Example 157 and Example 158

The following C. pneumoniae protein (PID 4376412) was expressed <SEQ ID 307; cp6412>:

```
    1 MSSSEVVFQT VHGLGFGGLS SKSVVPFKKS LSDAPRVVCS ILVLTLGLGA
  51 LVCGIAITCW CVPGVILMGG ICAIVLGAIS LALSLFWLWG LFSNCCGSKR
101 VLPGEGLLRD KLLDGGFSRA APSGMGLPGD GSPRASTPSC LEELQAEIQA
151 VTQAIDQMSD D*
```

The cp6412 nucleotide sequence <SEQ ID 308> is:

```
    1 ATGAGCAGTT CGGAAGTTGT TTTCCAGACA GTTCATGGCC TTGGCTTTGG
  51 TGGATTGTCT TCAAAAAGTG TTGTCCCTTT TAAGAAAAGT CTTTCGGATG
101 CGCCCCGTGT TGTGTGCTCG ATTTTAGTTT TGACTCTGGG GTTGGGAGCG
151 CTTGTTTGTG GTATTGCCAT TACTTGTTGG TGTGTCCCGG GAGTTATTTT
201 AATGGGGGGA ATTTGCGCTA TAGTTTTAGG TGCAATTTCT TTAGCTTTAA
251 GTCTATTTTG GTTGTGGGGT TTATTTTCTA ATTGTTGTGG TTCTAAGAGA
301 GTTTTACCGG GTGAGGGATT GCTACGGGAT AAGCTTTTAG ATGGTGGATT
351 TTCAAGAGCG GCACCTTCAG GAATGGGACT TGGCCCTGAT GGATCTCCAA
401 GAGCGTCAAC GCCATCTTGC CTAGAGGAAC TTCAAGCAGA GATACAGGCA
451 GTTACTCAAG CTATCGATCA GATGTCAGAT GATTGA
```

The PSORT algorithm predicts inner membrane (0.4864).

The following C. pneumoniae protein (PID 4376431) was also expressed <SEQ ID 309; cp6431>:

```
    1 LRAGGSLVTT YPKEGQRLRS PEQLRVLDDL VQSYPNHLHA IELDCGAIPQ
  51 DLIGATYIIT FADFSTYILS LRSYQANSPS DDTWGIWFGS IDDPVQAVIS
101 FLKDHGFALP STLAQDPLLC TNK*
```

The cp6431 nucleotide sequence <SEQ ID 310> is:

```
    1 TTGCGAGCAG GAGGTAGTCT TGTTACAACA TACCCTAAGG AAGGTCAGAG
  51 ATTGCGCTCC CCAGAACAGT TAAGAGTTCT GGATGATTTA GTGCAAAGCT
101 ATCCAAATCA CCTACATGCG ATTGAACTTG ATTGTGGTGC AATCCCTCAA
151 GATTTGATCG GAGCCACCTA TATCATCACG TTCGCCGATT TTTCCACCTA
201 TATTCTCTCT TTAAGAAGCT ACCAAGCCAA TTCTCCCTCC GATGATACAT
251 GGGGGATTTG GTTTGGATCT ATTGACGATC CTGTTCAAGC AGTCATATCA
301 TTTTTAAAAG ATCATGGATT TGCTCTTCCC TCGACCTTAG CTCAAGATCC
351 TTTGCTTTGT ACTAACAAGT AA
```

The PSORT algorithm predicts cytoplasm (0.2115).

The following C. pneumoniae protein (PID 4376443) was also expressed <SEQ ID 311; cp6443>:

```
    1 MIMTTISNSP SPALNPELSL IPPPTLVSSG TQTSLAYTIP AQGRRSTLRI
  51 ILDIFIIILG LATIISTFIV IFFLNGLNLL STPSIISSSC LIIVGLLFLI
101 NGLYFMISSL DQGLVGLLQK ELSQAEEREE EYIQEIEALR GAPRAESPTE
151 SPSTWL*
```

The cp6443 nucleotide sequence <SEQ ID 312> is:

```
    1 ATGATTATGA CTACTATATC TAACTCACCC TCCCCTGCAT TGAATCCCGA
  51 ACTTTCCCTT ATTCCTCCAC CAACACTTGT ATCTTCAGGT ACGCAAACAT
101 CTCTAGCTTA TACGATCCCC GCACAAGGAC GAAGATCCAC CCTACGTATT
151 ATATTAGATA TATTCATTAT CATTCTTGGT TTAGCTACGA TCATTTCTAC
201 CTTTATTGTT ATTTTCTTTT TAAATGGGCT GAACTTGCTC TCGACCCCAT
251 CTATTATCTC TTCGTCATGT TTAATCATTG TTGGATTGCT TTTTTTGATT
301 ATGGGGTTAT ATTTCATGAT CTCGAGTTTG GATCAGGGGC TTGTAGGCCT
351 TCTGCAAAAG GAACTCTCTC AAGCCGAAGA AAGAGAAGAA GAGTATATCC
401 AGGAAATCGA AGCTTTAAGA GGAGCTCCTA GAGCAGAATC TCCCACAGAG
451 TCTCCTAGTA CCTGGTTATG A
```

The PSORT algorithm predicts inner membrane (0.5585).

The following C. pneumoniae protein (PID 4376496) was also expressed <SEQ ID 313; cp6496>:

```
    1 MLIGRYSSDD QFTEATKNTP TIIKLGFVRD NLEGLTNPIS EIVSETSSSI
  51 KDSVLRSIPI LGSILGCARL YSTLSTNDPL DETQEKIWHT IFGALETLGL
101 GILILLFKII FVILHCIFHL VIGFCK*
```

The cp6496 nucleotide sequence <SEQ ID 314> is:

```
    1 ATGCTAATAG GCAGATACAG TAGTGATGAC CAATTCACTG AAGCAACAAA
  51 AAACACCCCA ACCATAATTA AGCTAGGTTT TGTTAGAGAT AATCTCGAGG
101 GATTAACGAA CCCTATCTCT GAAATCGTCT CGGAAACCTC CTCTTCTATT
151 AAAGATTCCG TTCTTCGCTC TCTTCCTATT TTAGGGTCCA TTTTAGGATG
201 CGCCCGACTT TACAGCACAC TCTCTACAAA TGATCCTCTT GACGAAACTC
251 AAGAAAAGAT TTGGCACACT ATATTTGGAG CCTTAGAAAC CTTAGGCTTA
301 GGGATTCTCA TCCTCTTATT TAAAATTATT TTTGTTATAT TACACTGCAT
351 ATTTCATCTA GTTATTGGGT TCTGCAAATA A
```

The PSORT algorithm predicts inner membrane (0.5989).

The following C. pneumoniae protein (PID 4376654) was also expressed <SEQ ID 315; cp6654>:

```
    1 MKTKMNSRKK AGQWAIFNSP TPGVSSTLVL AWTPWGYYDK DVQDILERKD
  51 PMSSSLSEKD SKEFLKNLFV DLLENGFTSV HIHAEEAFTP LDHTGKPHFK
101 RDNVYLPGKL LGALNEAAVQ ANVSADTQFT LFLTQDECNP FHDKKRG*
```

The cp6654 nucleotide sequence <SEQ ID 316> is:

```
    1 ATGAAAACTA AAATGAACTC TAGAAAAAAA GCAGGTCAAT GGGCAATTTT
  51 CAATTCTCCA ACTCCTGGTG TCAGTTCAAC TTTAGTTTTA GCATGGACTC
101 CTTGGGGTTA TTACGACAAG GATGTACAAG ATATCTTAGA AAGAAAAGAT
151 CCGATGAGCT CTTCGCTTTC TGAAAAAGAC TCAAAGGAGT TCTTGAAAAA
201 TCTGTTTGTA GATCTCTTAG AAAATGGCTT CACATCAGTA CATATTCACG
251 CAGAAGAAGC TTTCACTCCT CTTGATCATA CCGGGAAACC TCACTTTAAA
301 AGAGACAATG TGTACTTACC CGGAAAGTTG TTAGGCGCCT TGAATGAGGC
351 TGCGGTACAA GCCAATGTAA GTGCGGATAC TCAATTTACA TTGTTCCTTA
401 CTCAAGATGA GTGCAATCCT TTTCATGATA AGAAAAGAGG TTAA
```

The PSORT algorithm predicts cytoplasm (0.0730).

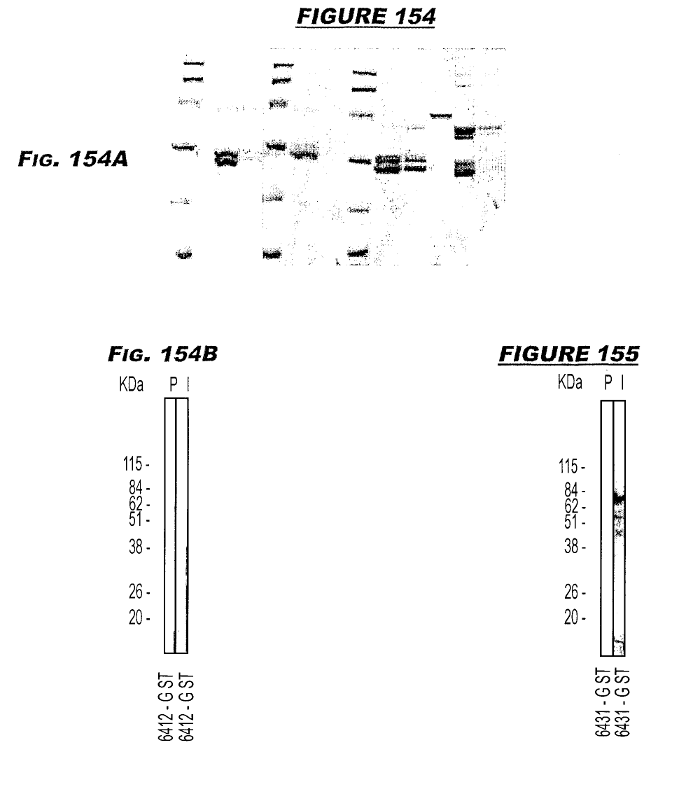

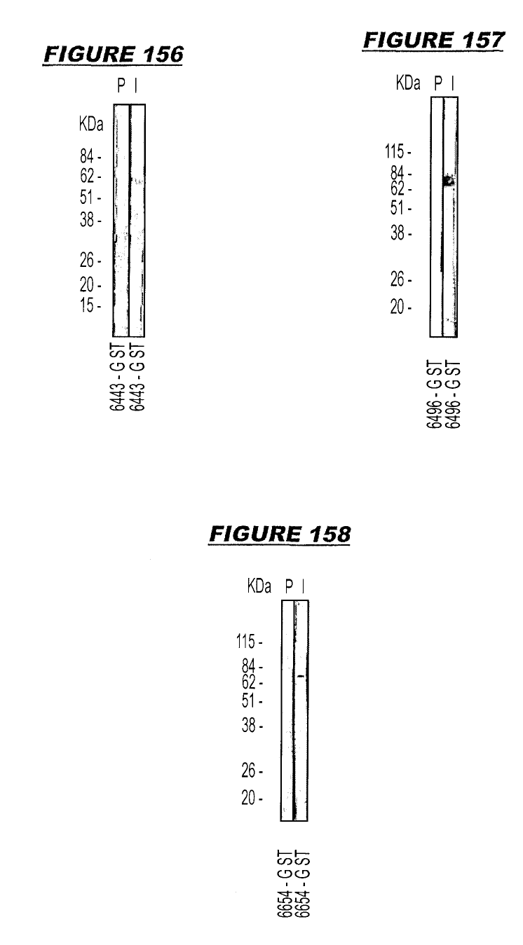

The proteins were expressed in E. coli and purified as his-tag products (FIG. 154A; 6412=lanes 2-3; 6431=lanes 11-12; 6443=lanes 5-6; 6496=lanes 8-9; 6654=lane 10; markers in lanes 1, 4, 7). The recombinant proteins were used to immunise mice, whose sera were used in Western blots (FIGS. 154B, 155, 156, 157 & 158) and for FACS analysis.

These experiments show that cp6412, cp6431, cp6443, cp6496 & cp6654 are surface-exposed and immunoaccessible proteins and that they are useful immunogens. These properties are not evident from their sequences alone.

### Example 159 and Example 160

The following C. pneumoniae protein (PID 4376477) was expressed <SEQ ID 317; cp6477>:

1 LLKFFLVCEE LCILTVATHR ALLETPLALS FFKELKTKYV YRAKDILQLH
51 NYKGFTIINT SPLCS*

The cp6477 nucleotide sequence <SEQ ID 318> is:

```
    1 TTGCTAAAGT TCTTTCTAGT ATGTGAAGAG TTATGTATAC TTACTGTTGC
  51 TACACATAGA GCTCTCTTAG AAACTCCTTT AGCTCTATCA TTTTTTAAAG
101 AACTTAAGAC AAAATATGTC TACAGGGCGA AAGACATACT ACAACTACAT
151 AACTATAAAG GATTTACTAT CCTTAATACA TCACCGTTAT GTTCTTAA
```

The PSORT algorithm predicts inner membrane (0.128).

The following C. pneumoniae protein (PID 4376435) was also expressed <SEQ ID 319; cp6435>:

```
    1 LWSHFPRGFF MLPFCPTILL AKPFLNSENY GLERLAATVD SYFDLGQSQI
  51 VFLSKQDQGI TVEELSAKDR KFKPGSMNCT LYTEDPILPA HNSFSNCSDI
101 QMRTPISPIH *
```

The cp6435 nucleotide sequence <SEQ ID 320> is:

```
    1 TTGTGGTCGC ATTTCCCAAG AGGATTTTTT ATGCTCCCTT TTTGCCCTAC
  51 CATCCTTCTT GCTAAACCTT TTTTAAATAG CGAGAATTAC GGCTTAGAAC
101 GTTTAGCTGC AACCGTAGAT TCTTATTTTG ATCTGGGACA GTCTCAAATA
151 GTCTTCCTAA GCAAACAGGA TCAAGGAATC ACTGTGGAAG AATTGAGTGC
201 TAAAGATAGG AAATTCAAGC CAGGCTCTAT GAACTGTACA CTGTACACTG
251 AAGATCCTAT CTTACCTGCT CATAATTCCT TTAGTAATTG CTCTGATATT
301 CAAATGCGTA CTCCGATTAG CCCTATACAT TAA
```

The PSORT algorithm predicts periplasmic space (0.4044).

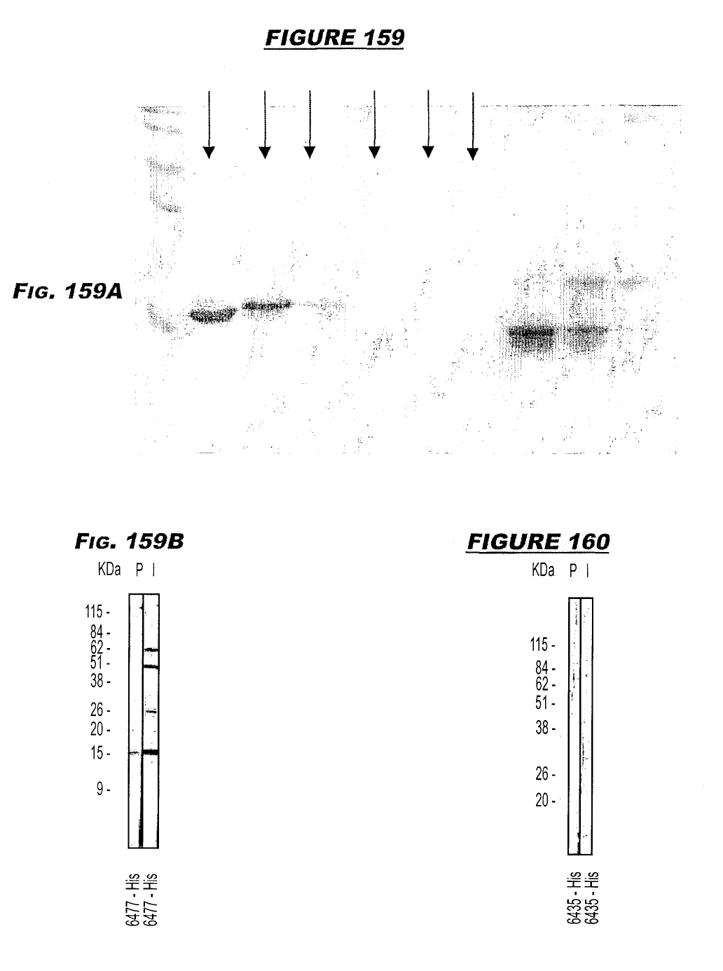

The proteins were expressed in E. coli and purified as his-tag products (FIG. 159A; 6435=lanes 2-4; 6477=lanes 5-7). The recombinant proteins were used to immunise mice, whose sera were used in Western blots (FIGS. 159B & 160) and for FACS analysis.

These experiments show that cp6477 & cp6435 are surface-exposed and immunoaccessible proteins and that they are useful immunogens. These properties are not evident from the sequences alone.

### Example 161 and Example 162 and Example 163

The following C. pneumoniae protein (PID 4376441) was expressed <SEQ ID 321; cp6441>:

```
    1 VEAGANVLVI DTAHAHSKGV FQTVLEIKSQ FPQISLVVGN LVTAFAAVSL
  51 AEIGVDAVKV GIGPGSICTT RIVSGVGYPQ ITAITNVAKA LKNSAVTVIA
101 DGRIRYSGDV VKALAAGADC VMLGSLLAGT DEAPGDIVSI DEFLFKRYRG
151 MGSLGAMKQG SADRYFQTQG QKKLVPGGVE GLVAYKGSVH DVLYQILGGI
201 RSGMGYVGAE TLKDIKTKAS FVRITESGRA ESHIHNIYKV QPTLNY
```

The cp6441 nucleotide sequence <SEQ ID 322> is:

```
    1 GTGGAAGCTG GAGCAAATGT TCTAGTCATT GACACAGCTC ATGCACACTC
  51 TAAAGGAGTA TTCCAAACAG TTTTAGAAAT AAAATCCCAG TTCCCACAAA
101 TTTCTTTAGT TGTAGGGAAT CTTGTTACAG CTGAAGCCGC AGTTTCCTTA
151 GCTGAGATTG GAGTTGACGC TGTAAAGGTA GGTATTGGCC CAGGATCTAT
201 CTGTACAACT AGAATCGTTT CAGGGGTCGG TTATCCACAA ATTACTGCCA
251 TTACAAACGT AGCAAAAGCT CTTAAAAACT CTGCCGTGAC TGTAATTGCT
301 GATGGGAGAA TCCGCTATTC TGGAGATGTG GTAAAAGCAT TAGCAGCAGG
351 AGCAGACTGT GTCATGCTAG GAAGTTTGCT TGCAGGGACT GATGAAGCTC
401 CTGGGGATAT CGTTTCTATC GATGAGAAGC TTTTTAAAAG GTACCGCGGC
451 ATGGGATCTT TAGGCGCTAT GAAACAAGGA AGTGCTGACC GGTATTTTCA
501 AACACAGGGA CAGAAAAAGC TGGTTCCTGG GGGAGTTGAA GGACTAGTCG
551 CTTATAAAGG CTCTGTCCAC GATGTCCTCT ATCAAATTTT AGGAGGAATA
601 CGCTCAGGTA TGGGGTATGT TGGAGCTGAA ACTCTCAAAG ATTTAAAAAC
651 TAAGGCTTCC TTTGTTCGAA TTACTGAATC TGGAAGAGCT GAAAGTCATA
701 TTCATAATAT TTACAAAGTT CAACCAACCT TAAATTATTA A
```

The PSORT algorithm predicts bacterial inner membrane (0.132).

The following C. pneumoniae protein (PID 4376748) was also expressed <SEQ ID 323; cp6748>:

```
    1 LFSFGTAINL FRIFAPLRNR VTTEYSRARQ PDLHRIAIVY IGVLDSESSK
  51 ILERLISYMS CIMSESQMYL RFFMGKNVNQ SAVLSKLEVE NLHIRCGFFS
101 EDAVPESEPF DLSIYVHTDR SCPLPTKKRS SSWELQTVEL PESIYPQSFF
151 LLMRPRMLS*
```

The cp6748 nucleotide sequence <SEQ ID 324> is:

```
    1 TTGTTCTCTG AGGGGACAGC TCTAAATTTA TTTCGTATAT TTGCTCCACT
  51 ACGCAACCGT GTGACTACAG AATACAGTCG TGCTAGGCAA CCCGACCTAC
101 ATAGAATTGC CATCGTCTAT ATAGGAGTTC TCGATTCAGA AAGTTCCAAG
151 ATCCTAGAGC GGCTAATCTC TTATATGAGT TGTATCTATT CTGAATCGCA
201 AATGTATTTA AGATTCTTTA TGGGCAAGAA TGTAAATCAA AGTGCTGTAC
251 TCTCAAAATT ACATGTAGAA AATCTGCACA TCCGTTGTGG GTTTTTCAGC
301 GAGGATGCTG TTCCAGAGAG TGAGCCCTTC GATCTCTCCA TCTACGTGCA
351 CACAGATCGT AGCTGTCCTC TCCCTACGAA AAAACGGAGC AGCTCCTGGG
401 AACTCCAAAC TGTAGAACTC CCAGAGTCAA TATATCCACA GTCGGAATTC
451 CTATTGATGA GACCTCGAAT GCTTTCGTAG
```

The PSORT algorithm predicts cytoplasm (0.170).

The following C. pneumoniae protein (PID 4376881) was also expressed <SEQ ID 325; cp6881>:

```
    1 MRPHRKHVSS KSLALKQSAS THVEITTKAF RLSMPLKQLI LEKSDHLPPM
  51 ETIRVVLTSH KDKLGTEVHV VASHGKEILQ TKVENANPYT AVINAFKKIR
101 TMANKHSNKR KDRTKHDLGL AAKEERIAIQ EEQEDRLSNE WLPVFGLDAW
151 DSLKTLGYVP ASAKKKISKK KMSIRMLSQD EAIRQLESAA ENFLIFLNEQ
201 EHKIQCIYKK HDGNYVLIEP SLKPGFCI*
```

The cp6881 nucleotide sequence <SEQ ID 326> is:

```
    1 ATGAGACCTC ATCGTAAACA CGTATCATCT AAAAGCTTAG CTTTAAAGCA
  51 ATCTGCATCA ACTCATGTAG AGATCACAAC AAAAGCCTTT CGTCTCTCTA
101 TGCCTCTAAA ACAGCTGATC CTAGAGAAAA GCGACCACCT CCCCCCTATG
151 GAAACAATCC GTGTGGTGCT AACCTCTCAT AAAGATAAGC TAGGCACCGA
201 GGTGCATGTT GTAGCTTCTC ATGGCAAAGA AATCCTTCAA ACTAACCTTC
251 ATAACGCAAA CCCATACACT GCAGTGATCA ATGCTTTTAA GAAAATCCGC
301 ACCATGGCAA ATAAGCACTC CAATAAACGT AAAGACAGGA CAAAACATGA
351 TCTAGGTCTT GCAGCAAAAG AAGAACGTAT CGCAATACAG GAAGAACAAG
401 AAGATCGCCT TAGCAACGAG TGGCTTCCTG TCGAAGGCCT CGATGCCTGG
451 GATTCTCTAA AAACTCTTGG GTATGTTCCC GCATCAGCGA AAAAGAAGAT
501 CTCCAAGAAA AAGATGAGCA TTCGTATGCT ATCTCAAGAC GAGGCTATCC
551 GCCAGCTAGA GTCTGCCGCA GAAAACTTCC TGATCTTCTT GAACGAGCAA
601 GAGCATAAAA TCCAATGCAT TTATAAAAAA CATGACGGCA ACTATGTCCT
651 TATTGAACCT TCCCTCAAGC CAGGATTCTG CATCTGA
```

The PSORT algorithm predicts cytoplasm (0.249).

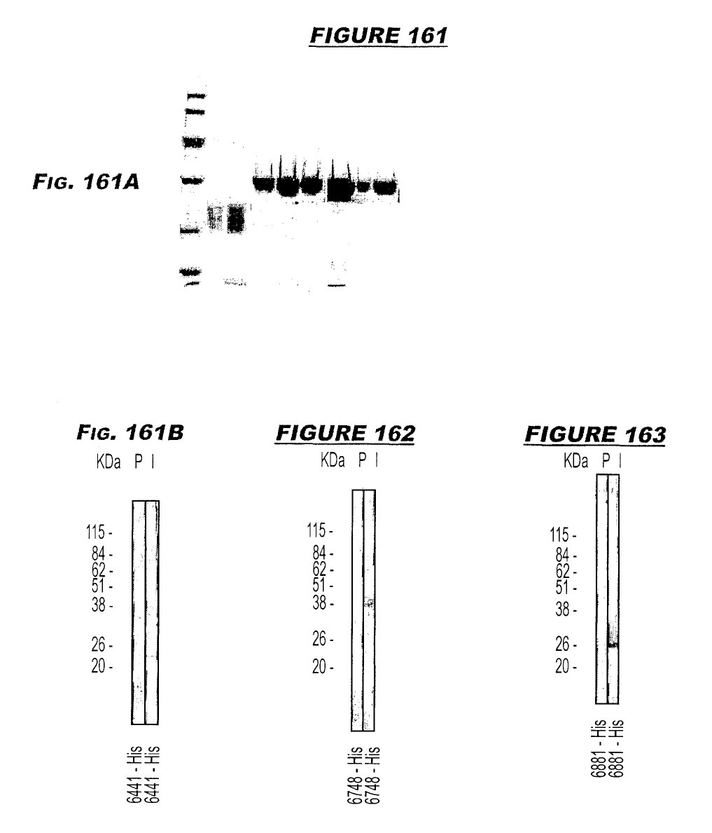

The proteins were expressed in E. coli and purified as his-tag products (FIG. 161A; 6441=lanes 7-9; 6748=lanes 2-3; 6881=lanes 4-6). The recombinant protein was used to immunise mice, whose sera were used in Western blots (FIGS. 161B, 162 & 163) and for FACS analysis.

These experiments show that cp6441, cp6748 & cp6881 are surface-exposed and immunoaccessible proteins and that they are useful immunogens. These properties are not evident from the sequence alone.

### Example 164 and Example 165 Example 166

The following C. pneumoniae protein (PID 4376444) was expressed <SEQ ID 327; cp6444>:

```
    1 MEQPNCVIQD TTTVLYALNS FDPRLSDDTH RLGKQSPLEA ENALGEFIEG
  51 LDTNSFPLEE VAIPILPGYH PKFYLSFIDR DDQGVHYEVL DGVFLKTVAA
101 CIIENSFLTD SMSPELLSEV KEALKR*
```

The cp6444 nucleotide sequence <SEQ ID 328> is:

```
    1 ATGGAGCAAC CCAATTGTGT GATTCAGGAT ACTACAACTG TTTTGTATGC
  51 CTTAAATAGC TTTGATCCTA GACTTAGTGA TGACACTCAC AGACTTGGGA
101 AGCAATCACC TCTTGAAGCA GAAAATGCTC TTGGAGAATT TATTGAAGGT
151 TTGGATACAA ATAGCTTTCC TTTAGAGGAA GTTGCCATTC CCATCCTGCC
201 AGGTTATCAC CCTAAGTTTT ATTTATCTTT CATAGATAGG GACGATCAAG
251 GTGTCCACTA TGAAGTTTTA GATGGCGTAT TTTTAAAGAC AGTCGCTGCT
301 TGTATTATAG AGAACTCCTT CTTAACTGAT TCTATGAGCC CGGAGCTTCT
351 CAGCGAAGTT AAGGAAGCTC TGAAACGATG A
```

The PSORT algorithm predicts cytoplasm (0.2031).

The following C. pneumoniae protein (PID 4376413) was also expressed <SEQ ID 329; cp6413>:

```
    1 MAVQSIKEAV TSAATSVGCV NCSREAIPAF NTEERATSIA RSVIAAIIAV
  51 VAISLLGLGL VVLAGCCPLG MAAGAITMLL GVALLAWAIL ITLRLLNIPK
101 AEIPSPGNNG EPNERNSATP PLEGGVAGEA GRGGGSPLTQ LDLNSGAGS*
```

The cp6413 nucleotide sequence <SEQ ID 330> is:

```
    1 ATGGCTGTTC AATCTATAAA AGAAGCCGTA ACATCAGCCG CAACATCAGT
  51 AGGATGTGTA AACTGTTCTA GAGAGGCTAT ACCAGCATTT AATACAGAGG
101 AGAGAGCAAC GAGTATTGCT AGATCTGTTA TAGCAGCTAT CATTGCTGTT
151 GTAGCTATCT CCTTACTCGG ACTAGGTCTT GTAGTTCTTG CTGGTTGCTG
201 TCCTTTAGGA ATGGCTGCGG GTGCTATAAC AATGCTGCTG GGTGTAGCAT
251 TATTAGCTTG GGCAATACTG ATTACTTTGA GACTGCTTAA TATACCTAAG
301 GCTGAAATAC CGAGTCCAGG GAACAACGGT GAGCCTAATG AAAGAAATTC
351 AGCAACTCCT CCTCTAGAGG GTGGTGTTGC AGGAGAAGCC GGTCGCGGCG
401 GGGGGTCACC TTTAACCCAA CTTGATCTCA ATTCAGGGGC GGGAAGTTAG
```

The PSORT algorithm predicts inner membrane (0.6180).

The following C. pneumoniae protein (PID 4377391) was also expressed <SEQ ID 331; cp7391>:

```
    1 MMLRVIELPL IPIKQALEKA FVQYNSYKAK LTKVEPCFRE SPAYITSEER
  51 LQSLDQTLER AYKEYQKRFQ EPSRLESEVS GCREHLREQV KQFETQGLDL
101 IKEFLIFVSD VLFRKMVSCL VSTVHVPFME FYYEYFELHR LRLRAQWMAN
151 AEIYSKVRKA FPEMLKETLE KAKAPREEEY WLLCEERKSK EKRLILNKIE
201 AAQQRVKDLE PPPIKETGKQ KRKKEYSFFI RLKS*
```

The cp7391 nucleotide sequence <SEQ ID 332> is:

```
    1 ATGATGCTTC GTGTCATAGA GCTTCCACTA CTTCCTATAA AGCAAGCGTT
  51 GGAGAAGGCT TTTGTACAAT ATAATAGCTA CAAAGCGAAG TTAACCAAGG
101 TAGAACCTTG CTTTAGAGAG AGCCCTGCCT ATATAACTAG CGAAGAGCGA
151 CTCCAGAGTT TGGATCAGAC TTTAGAACGT GCGTACAAAG AGTACCAGAA
201 GAGATTCCAG GAGCCTTCAC GTTTGGAATC GGAAGTAAGT GGATGTAGAG
251 AGCATCTTAG AGAGCAGGTA AAACAATTTG AAACTCAAGG ACTAGACTTG
301 ATCAAAGAAG AGCTTATTTT TGTTAGTGAT GTGTTATTCC GAAAAATGGT
351 CAGTTGTCTA GTGTCGACAG TGCATGTTCC CTTTATGGAG TTTTATTATG
401 AGTATTTTGA GTTGCATAGA TTGAGGTTGC GGGCCCAATG GATGGCGAAT
451 GCCGAGATTT ATAGCAAAGT TAGAAAAGCA TTCCCAGAGA TGTTGAAGGA
501 GACCTTAGAA AAAGCTAAGG CTCCCAGAGA AGAAGAGTAT TGGTTACTTT
551 GCGAGGAGAG AAAGAGTAAG GAGAAGCGTT TGATTCTCAA CAAGATAGAG
601 GCAGCTCAGC AGCGGGTAAA AGATTTAGAA CCTCCTCCTA TTAAAGAGAC
651 AGGGAAACAG AAACGGAAGA AAGAATATTC GTTTTTCATT CGATTAAAAT
701 CGTGA
```

The PSORT algorithm predicts inner membrane (0.1489).

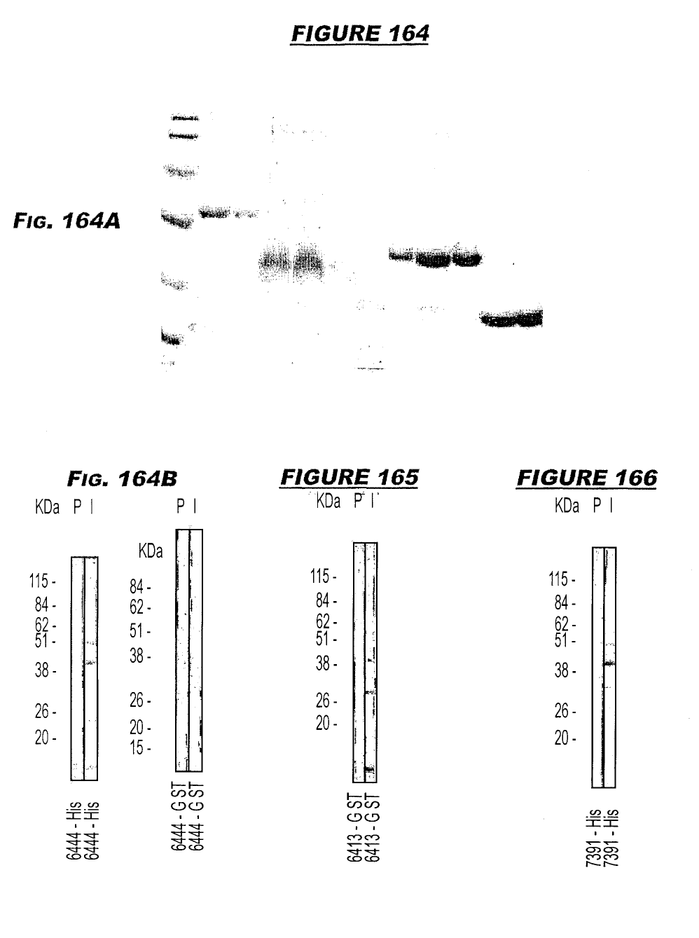

The proteins were expressed in E. coli and purified as his-tag and GST-fusion products (FIG. 164A; 6444=lanes 11-12; 7391=lanes 2-3; 6413=lanes 4-6). The recombinant protein was used to immunise mice, whose sera were used in Western blots (FIGS. 164B, 165 & 166) and for FACS analysis.

These experiments show that cp6444, cp6413 & cp7391 are surface-exposed and immunoaccessible proteins and that they are useful immunogens. These properties are not evident from the sequence alone.

### Example 167, Example 168, Example 169 and Example 170

The following C. pneumoniae protein (PID 4376463) was expressed <SEQ ID 333; cp6463>:

```
    1 MKKKVTIDEA LKEILRLEGA ATQEELCAKL LAQGFATTQS SVSRWLRKIQ
  51 AVKVAGERGA RYSLPSSTEK TTTRHLVLSI RHNASLIVIR TVPGSASWIA
101 ALLDQGLKDE ILGTLAGDDT IFVTPIDEGR LPLLMVSIAN LLQVFLD*
```

The cp6463 nucleotide sequence <SEQ ID 334> is:

```
    1 ATGAAAAAAA AAGTAACTAT AGATGAGGCT TTAAAAGAAA TTTTACGTCT
  51 TGAAGGAGCG GCAACTCAGG AGGAATTATG TGCAAAACTC TTAGCTCAAG
101 GTTTTGCTAC AACCCAGTCG TCTGTATCTC GTTGGCTACG AAAGATTCAG
151 GCTGTAAAGG TTGCTGGAGA GCGTGGTGCT CGTTATTCTT TACCCTCTTC
201 AACAGAGAAG ACCACGACCC GTCATTTGGT GCTCTCTATT CGCCATAACG
251 CCTCTCTTAT TGTAATTCGT ACGGTTCCTG GTTCAGCTTC TTGGATCGCT
301 GCTTTGTTAG ATCAAGGGCT CAAAGATGAA ATTCTTGGAA CTTTGGCAGG
351 AGATGACACG ATTTTTGTCA CTCCTATAGA TGAAGGGAGG CTCCCATTGT
401 TGATGGTTTC GATTGCAAAT TTACTGCAAG TTTTCTTGGA TTAA
```

The PSORT algorithm predicts inner membrane (0.1510).

The following C. pneumoniae protein (PID 4376540) was also expressed <SEQ ID 335; cp6540>:

```
    1 MSQCQSSSTS TWEWMKSFVP NWKNPTPPLS PIPSEDEFIL AYEPFVLPKT
  51 DPENAQANPP GTSTPNVENG IDDLNPLLGQ PENQNNANNP GTSGSNPTSL
101 PAPERLPETE ENSQEEEQGS QNNEDLIG*
```

The cp6540 nucleotide sequence <SEQ ID 336> is:

```
    1 ATGTCTCAAT GTCAGAGTAG CAGTACATCT ACCTGGGAAT GGATGAAATC
  51 TTTTGTGCCA AACTGGAAGA ATCCAACTCC CCCCTTATCT CCTATACCTT
101 CTGAGGACGA ATTTATATTA GCATACGAGC CATTTGTTCT ACCGAAAACA
151 GATCCAGAAA ACGCACAAGC TAATCCTCCA GGCACATCTA CACCGAATGT
201 AGAAAACGGG ATCGATGATC TCAACCCTCT TCTGGGGCAA CCCAACGAAC
251 AAAACAATGC CAACAATCCA GGAACTTCTG GATCTAATCC TACATCTCTA
301 CCCGCCCCCG AACGACTCCC TGAAACTGAA GAGAACAGCC AAGAAGAAGA
351 ACAAGGATCT CAAAATAATG AGGATCTTAT AGGATAA
```

The PSORT algorithm predicts cytoplasm (0.3086).

The following C. pneumoniae protein (PID 4376743) was also expressed <SEQ ID 337; cp6743>:

```
    1 LREFGSVSFR EYFRAYMCDK IVAQKNFLFT LDAVIKQAGW RSQEKLNLFY
  51 VESQALGREI KVSLEEYIQS MVGILGSQRT KKSFKFSVDF TPLEQALQER
101 CSSDDDEDAT ATSTATGATA SPTDMHEDE*
```

The cp6743 nucleotide sequence <SEQ ID 338> is:

```
    1 TTGAGAGAAG AAGGTAGTGT TTCTTTCAGA GAATATTTCA GAGCCTATAT
  51 GTGTGATAAA ATCGTGGCAC AGAAGAACTT CTTATTTACT TTAGACGCTG
101 TAATTAAACA GGCCGGTTGG AGATCACAAG AGAAACTCAA TTTATTTTAT
151 GTTGAAAGTC AGGCTTTAGG AAGAGAAATC AAAGTCAGCT TAGAGGAATA
201 TATTCAGAGT ATGGTCGGGA TTTTGGGATC TCAGAGAACC AAGAAAAGCT
251 TTAAGTTTTC TGTCGACTTT ACCCCTTTAG AGCAGGCTCT ACAAGAAAGA
301 TGCTCTTCTG ATGATGACGA AGATGCAACA GCAACTTCGA CCGCTACAGG
351 GGCAACAGCA TCTCCGACTG ACATGCACGA AGATGAGTAA
```

The PSORT algorithm predicts cytoplasm (0.2769).

The following C. pneumoniae protein (PID 4377041) was also expressed <SEQ ID 339; cp7041>:

```
    1 MLMMLMMIIG ITGGSGAGKT TLTQNIKEIP GEDVSVICQD NYYKDRSHYT
  51 PEERANLIWD HPDAFDNDLL ISDIKRLKNN EIVQAPVFDF VLGNRSKTEI
101 ETIYPSKVIL VEGILVFENQ ELRDLMDIRI FVDTDADERI LRRMVRDVQE
151 QGDSVDCIMS RYLSMVKPMH EKFIEPTRKY ADIIVHGNYR QNVVTNILSQ
201 KIKNHLENAL ESDETYYMVN SK*
```

The cp7041 nucleotide sequence <SEQ ID 340> is:

```
    1 ATGTTGATGA TGCTTATGAT GATTATTGGA ATTACAGGAG GTTCTGGAGC
  51 TGGGAAAACC ACCCTAACCC AAAACATTAA AGAAATTTTC GGTGAGGATG
101 TGAGTGTTAT CTGCCAAGAT AATTATTACA AAGATAGATC TCATTATACT
151 CCTGAAGAAC GTGCCAATTT AATTTGGGAT CATCCGGACG CCTTTGATAA
201 TGACTTATTA ATTTCAGACA TAAAACGTCT AAAAAATAAT GAGATTGTCC
251 AAGCCCCAGT TTTTGATTTT GTTTTAGGTA ATCGATCTAA AACGGAGATA
301 GAAACGATCT ATCCATCTAA AGTTATTCTT GTTGAAGGTA TTCTGGTCTT
351 TGAAAATCAA GAACTTAGAG ATCTTATGGA TATTAGGATC TTTGTAGACA
401 CCGATGCTGA TGAAAGGATA CTACGCCGTA TGGTTCGAGA TGTTCAAGAA
451 CAAGGAGATA GCGTGGACTG CATCATGTCT CGTTATCTTT CTATGGTAAA
501 GCCTATGCAT GAGAAATTTA TAGAGCCGAC TCGGAAATAT GCTGATATCA
551 TTGTACATGG AAATTACCGA CAAAACGTAG TAACAAATAT TTTGTCACAG
601 AAAATTAAAA ATCATTTAGA GAATGCCCTG GAAAGCGATG AGACGTATTA
651 TATGGTCAAC TCTAAGTAA
```

The PSORT algorithm predicts inner membrane (0.1022).

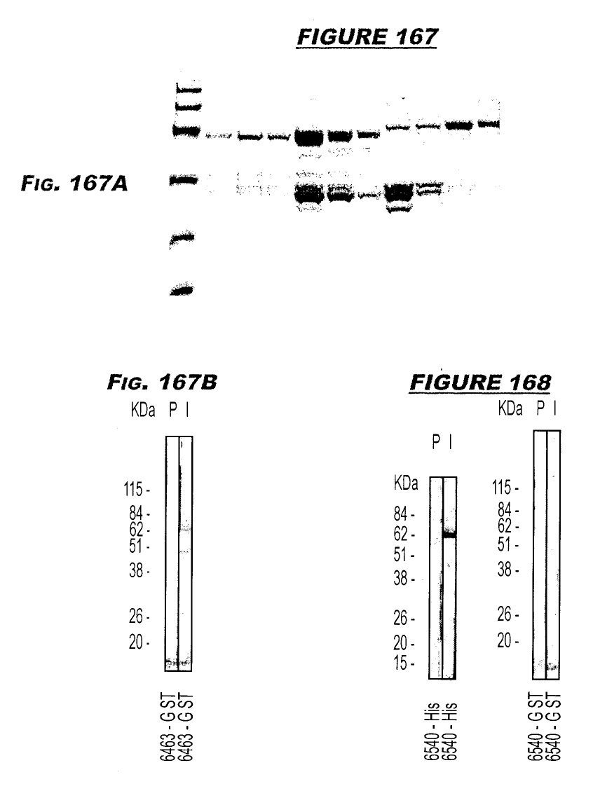

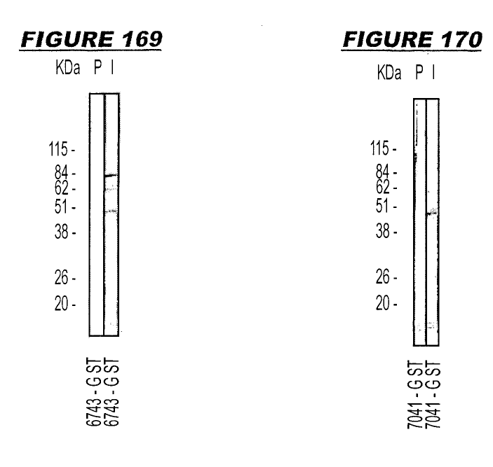

The proteins were expressed in E. coli and purified as his-tag products (FIG. 167A; 6463=lanes 2-4; 6540=lanes 5-7; 6743=lanes 8-9; 7041=lanes 10-11). The recombinant proteins were used to immunise mice, whose sera were used in Western blots (FIGS. 167B, 168, 169 & 170) and for FACS analysis.

These experiments show that cp6463, cp6540, cp6743 & cp7041 are surface-exposed and immunoaccessible proteins and that they are useful immunogens. These properties are not evident from the sequence alone.

### Example 171 and Example 172 and Example 173

The following C. pneumoniae protein (PID 4376632) was expressed <SEQ ID 341; cp6632>:

```
    1 VQLFQYMNES GWDWICDFDS QGEGFQLSRL VGLLHSSWAL YEAKFQFYLP
  51 EVSLLTWEEL IEMQLLSKPT KHGVAKDLCN VFEKHFQRFR QYLGSLDLNQ
101 RFENTFLNYP KYHLDRE*
```

The cp6632 nucleotide sequence <SEQ ID 342> is:

```
    1 GTGCAATTAT TTCAATATAT GAATGAGTCC GGATGGGATT GGCTTTGTGA
  51 TTTTGATTCT CAAGGCGAGG GATTCCAGTT ATCACGTCTG GTTGGGCTGT
101 TACATTCGTC CTGGGCATTA TACGAAGCAA AAGAGCAATT TTACCTTCCT
151 GAGGTTTCTC TATTGACCTG GGAAGAACTG ATAGAAATGC AGTTATTAAG
201 CAAACCAACA AAACACGGGG TTGCAAAAGA TCTTTGTAAT GTATTTGAAA
251 AACACTTTCA AAGGTTTAGA CAGTACCTAG GTTCCTTAGA TCTAAATCAA
301 AGGTTCGAAA ATACCTTCTT GAATTATCCT AAATACCATT TAGATAGGGA
351 GTGA
```

The PSORT algorithm predicts cytoplasm (0.3627).

The following C. pneumoniae protein (PID 4376648) was also expressed <SEQ ID 343; cp6648>:

```
    1 MPVSSAPLPT SHRPSSGNLG LMEPNSKALK AKHQDKTTKT IKLLVKILVA
  51 ILVIEVLGII AAFFIPGTPP ICLIILGGLI LTTVLCVLLL VIKLALVNKT
101 EGTTAEQQIK RKLSSKSIS*
```

The cp6648 nucleotide sequence <SEQ ID 344> is:

```
    1 ATGCCCGTGT CCTCAGCCCC CCTACCCACA AGCCACCGCC CTTCCTCTGG
  51 AAATCTAGGC CTCATGGAAC CAAATTCCAA AGCTCTAAAA GCAAAGCATC
101 AAGATAAAAC GACGAAGACG ATTAAACTTT TAGTTAAAAT CCTTGTTGCC
151 ATTCTAGTAA TAGAAGTTTT AGGAATAATT GCAGCTTTCT TTATTCCTGG
201 GACTCCTCCC ATCTGCTTGA TTATCCTAGG AGGCCTTATT CTTACAACAG
251 TACTCTGTGT GCTTCTTCTT GTTATAAAGC TTGCCCTTGT AAACAAAACC
301 GAAGGAACAA CTGCTGAACA GCAGATAAAA CGTAAACTCT CTTCTAAAAG
351 TATTTCTTAG
```

The PSORT algorithm predicts inner membrane (0.6074).

The following C. pneumoniae protein (PID 4376497) was also expressed <SEQ ID 345; cp6497>:

1 MKPNSIIFLE NTKHYPDIFR EGFVRDRHGL MEASDWLLST FITIIRSILG
51 AIPILGNILG AGRLYSVWYT SDEDWKKQVV *

The cp6497 nucleotide sequence <SEQ ID 346> is:

```
    1 ATGAAGCCAA ATAGTATTAT TTTTTTAGAA AATACTAAGC ATTATCCCGA
  51 CATCTTTCGA GAAGGATTTG TTCGTGATCG TCATGGACTA ATGGAAGCCT
101 CGGATTGGTT ACTTTCTACG GAAATTACGA TCATTCGCTC CATTCTGGGA
151 GCTATCCCTA TTTTAGGAAA TATTCTTGGA GCCGGACGAC TCTATAGCGT
201 TTGGTATACA AGTGACGAAG ATTGGAAAAA ACAAGTGGTT TGA
```

The PSORT algorithm predicts inner membrane (0.145).

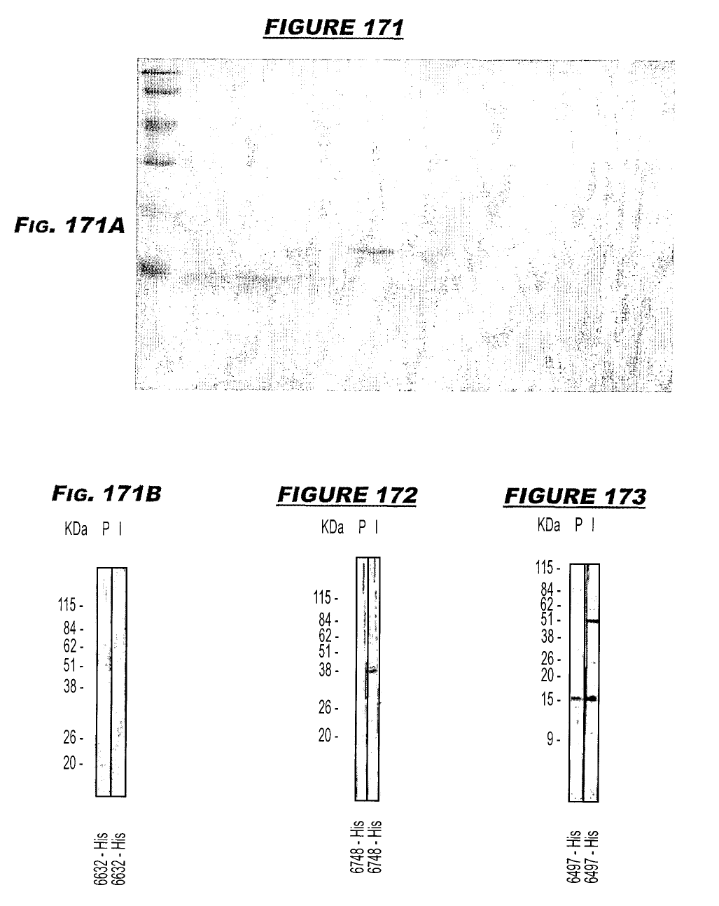

The proteins were expressed in E. coli and purified as his-tag products (FIG. 171A; 6632=lanes 5-7; 6648=lanes 8-10; 6497=lanes 2-4). The recombinant proteins were used to immunise mice, whose sera were used in Western blots (FIGS. 171B, 172, 173) and for FACS analysis.

These experiments show that cp6632, cp6648 and cp6497 are surface-exposed and immunoaccessible proteins and that they are useful immunogens. These properties are not evident from the sequence alone.

### Example 174, Example 175, Example 176, Example 177 and Example 178

The following C. pneumoniae protein (PID 4377200) was expressed <SEQ ID 347; cp7200>:

```
    1 MPVPIDNSSR NLQEVPESLE DLEQHAEESP THQSAESSSL QLSLASSAIS
  51 SRVFQLSSLV LGMENSDFSS LRDVPIFSAI YESSTHTPVP TPLVGVGYIN
101 GSQSGYYDTQ RESLHLSQLL GSRRVEVVYN QGNFMEASLL NLCPRRPRRD
151 PSPISLALLE LWEAFFLEHP PGSTFNPIFF W*
```

The cp7200 nucleotide sequence <SEQ ID 348> is:

```
    1 ATGCCCGTTC CTATAGATAA TTCCTCTCGC AACCTACAAG AAGTTCCAGA
  51 AAGCCTAGAA GACCTCGAAC AACACGCAGA AGAATCTCCT ACTCATCAAA
101 GTGCAGAAAG CAGTTCTTTG CAACTGTCTC TAGCCTCCTC AGCAATTTCT
151 AGTAGAGTAG AACAACTATC TTCCCTCGTC TTAGGAATGG AAAATTCAGA
201 TTTCTCCTCT TTAAGAGACG TTCCTATCTT CTCAGCTATC TACGAATCTT
251 CAACACACAC ACCTGTCCCC ACTCCTCTAG TTGGCGTGGG ATATATCAAC
301 GGAAGTCAAT CAGGATACTA CGATACACAA AGAGAATCTC TTCACCTCAG
351 CCAATTGTTA GGAAGCCGAA GAGTTGAAGT TGTCTATAAC CAAGGAAACT
401 TCATGGAGGC CTCTTTGCTA AATCTGTGCC CCAGAAGACC TCGAAGAGAT
451 CCCTCTCCAA TTTCTTTAGC TCTATTAGAG CTCTGGGAAG CATTTTTTTT
501 AGAACACCCC CCAGGTAGCA CTTTTAATCC AATATTTTTT TGGTAA
```

The PSORT algorithm predicts cytoplasm (0.3672).

The following C. pneumoniae protein (PID 4377235) was also expressed <SEQ ID 349; cp7235>:

```
    1 LNFVSTLTGS DFYAPVLEKL EEAFADTTGQ VILFSSSPDF IVHPIAQQLG
  51 ISSWYASCYR DQSAEQTIYK KCLTGDKKAQ ILSYIKKINQ ARSHTFSDHI
101 LDLPFLMLGE EKTVVRPQGR LKKMAKKYYW NIV*
```

The cp7235 nucleotide sequence <SEQ ID 350> is:

```
    1 TTGAATTTTG TATCGACTCT GACCGGCTCC GATTTTTATG CTCCTGTTTT
  51 AGAAAAACTA GAAGAAGCTT TTGCAGATAC CACAGGACAG GTGATCCTTT
101 TTTCTTCTTC TCCAGACTTT ATTGTCCACC CCATAGCGCA GCAACTCGGG
151 ATTAGTTCTT GGTATGCGTC GTGTTATCGC GATCAGTCTG CAGAACAGAC
201 GATCTATAAA AAATGTCTTA CAGGGGATAA AAAAGCGCAA ATTTTGAGTT
251 ATATTAAAAA AATTAATCAA GCAAGAAGCC ATACCTTCTC CGACCATATT
301 TTAGATCTTC CTCTTCTTAT GCTGGGAGAA GAGAAAACCG TCGTTCGCCC
351 TCAGGGACGA CTCAAGAAAA TGGCAAAAAA ATATTACTGG AATATCGTTT
401 AA
```

The PSORT algorithm predicts cytoplasm (0.3214).

The following C. pneumoniae protein (PID 4377268) was also expressed <SEQ ID 351; cp7268>:

```
    1 MMHRYFIPLL ALLIFSPSLV RAELQPSENR KGGWPTQLSC AEGSQLFCKF
  51 EAAYNNAIEE GKPGILVFFS ERPTPEFADL TNGSFSLSTP IAKGFNVVVL
101 CPGLISPLDF FHKMDPVILY MGSFLEMFPE VEAVSGPRLC YILIDEQGGA
151 QCQAVLPLET KN*
```

The cp7268 nucleotide sequence <SEQ ID 352> is:

```
    1 ATGATGCACC GTTATTTTAT TCCTTTATTA GCACTTCTCA TTTTCTCTCC
  51 TTCTTTAGTC AGGGCAGAGC TACAACCAAG TGAAAACAGA AAAGGGGGGT
101 GGCCTACACA ACTTTCCTGT GCAGAAGGTT CGCAACTCTT CTGTAAATTC
151 GAAGCTGCCT ATAATAATGC AATTGAGGAA GGGAAACCTG GGATTTTAGT
201 CTTTTTCTCT GAGCGACCCA CACCAGAATT TGCCGACTTA ACGAATGGTT
251 CATTTTCTCT CTCTACGCCA ATCGCCAAGG GCTTTAATGT CGTTGTGTTA
301 TGCCCCGGGC TTATCAGTCC CTTAGACTTT TTCCACAAAA TGGATCCTGT
351 GATTCTCTAT ATGGGAAGTT TTCTAGAGAT GTTCCCTGAA GTGGAGGCAG
401 TTAGTGGCCC TCGCTTATGT TATATCTTAA TAGATGAACA GGGTGGGGCT
451 CAATGTCAGG CTGTCCTGCC TTTAGAAACA AAGAATTAG
```

The PSORT algorithm predicts inner membrane (0.1235).

The following C. pneumoniae protein (PID 4377375) was also expressed <SEQ ID 353; cp7375>:

```
    1 MQRIIIVGID TGVGKTIVSA ILARALNAEY WKPIQAGNLE NSDSNIVHFL
  51 SGAYCHPEAY RLHKPLSPHK AAQIDNVSIE ESHICAPKTT SNLIIETSGG
101 FLSPCTSKRL QGDVFSSWSC SWILVSQAYL GSINHTCLTV FAMRSRNLNI
151 LGMVVNGYPE DEEHWLTQEI KLPIIGTLAK EKEITKTIIS CYAEQWKEVW
201 TSNHQGIQGV SGTPSLNLH*
```

The cp7375 nucleotide sequence <SEQ ID 354> is:

```
    1 ATGCAACGTA TCATCATTGT AGGAATCGAC ACTGGCGTAG GAAAAACCAT
  51 TGTCAGTGCT ATCCTTGCTA GAGCACTTAA CGCAGAATAC TGGAAACCTA
101 TACAAGCAGG GAATCTAGAA AATTCAGATA GCAATATTGT TCATGAGCTA
151 TCGGGAGCCT ACTGTCATCC CGAAGCTTAT CGATTGCATA AGCCCTTGTC
201 TCCACACAAG GCAGCGCAAA TCGATAATGT AAGTATCGAA GAGAGTCATA
251 TTTGTGCGCC AAAAACAACT TCGAATCTGA TTATTGAGAC TTCAGGAGGA
301 TTTTTATCCC CCTGCACATC AAAAAGACTT CAGGGAGATG TGTTTTCTTC
351 TTGGTCATGT TCTTGGATTT TAGTGAGCCA AGCATATCTC GGAAGTATCA
401 ATCACACCTG TTTAACGGTA GAAGCAATGC GCTCACGAAA CCTCAATATC
451 TTAGGTATGG TGGTAAATGG GTATCCAGAG GACGAAGAGC ACTGGCTAAC
501 TCAAGAAATC AAGCTTCCTA TAATCGGGAC TCTTGCCAAG GAAAAAGAAA
551 TCACAAAGAC AATCATAAGC TGTTATGCCG AACAATGGAA GGAAGTATGG
601 ACAAGCAATC ATCAGGGAAT TCAGGGTGTA TCTGGCACCC CTTCACTCAA
651 TCTGCATTAG
```

The PSORT algorithm predicts cytoplasm (0.0049).

The following C. pneumoniae protein (PID 4377388) was also expressed <SEQ ID 355; cp7388>:

```
    1 MQVLLSPQLP FPPQHSVGSI SSPSKLRVLA ITFLVFGMLL LISGALFLTL
  51 GIPGLSAAIS FGLGIGLSAL GGVLMISGLL CLLVKREIPT VRPEFIPEGV
101 SLAPSEEPAL QAAQKTLAQL PKELDQLDTD IQEVFACLRK LKDSKYESRS
151 FLNDAKKELR VFDFVVEDTL SEIFELRQIV AQEGWDLNFL INGGRSLMMT
201 AESFSLDIFH VSKRIGYLPS GDVRGEGLKK SAKEIVARLM SLHCFIHKVA
251 VAFDRNSYAM AEKAFAKALG ALEESVYRSL TQSYRDKFLE SERAKIPWNG
301 HITWLRDDAK SGCAEKKLRD AEERWKKFRK AVFWVEEDGG FDINNLLGDW
351 GTVLDPYRQE RMDEITFHEL YEKTTFLKRL HRKCALAKTT FEKKRSKKNL
401 QAVEEANARR LKYVRDWYDQ EFQKAGERLE KLHALYPEVS VSIRFNKIQE
451 TRSNLEKAYE AIEENYRCCV REQEDYWKEE EKREAEFRER GNKILSPEFL
501 ESSLEQFDHG LKNFSEKLME LEGHILKLQK EATAEVENKI LSDAFSRLFI
551 VFEDVKEMPC RIEEIEKTLR MAELPLLPTK KAFEKACSQY NSCAEMLEKV
601 KPYCKESLAY VTSKERLVSL DEDLRRAYTE CQKRFQGDSG LESEVRACRE
651 QLRERIQEFE TQGLDLVEKE LLCVSSRLRN TECDCVSGVK KEAPPGKKFY
701 AQYYDEIYRV RVQSRWMTMS ERLREGVQAC NKMLKAGLSE EDKVLKEEFY
751 WLYREERKNK EKRLVGTKIV ATQQRVAAFE SIEVPEIPEA PEEKPSLLDK
801 ARSLFTREDH T
```

The cp7388 nucleotide sequence <SEQ ID 356> is:

```
      1 ATGCAAGTAC TTCTATCTCC GCAGCTACCC CCCCCCCCCC AACACTCTGT
    51 AGGGTCGATT TCTTCTCCAT CTAAACTTCG CGTTTTAGCG ATTACTTTTT
  101 TAGTTTTTGG TATGCTCTTA CTGATTTCAG GAGCTCTCTT TCTGACGTTA
  151 GGGATTCCAG GATTGAGTGC AGCAATTTCT TTTGGATTAG GCATCGGTCT
  201 CTCCGCATTA GGAGGAGTGC TGATGATTTC GGGACTACTA TGTCTTTTAG
  251 TAAAACGAGA GATTCCGACA GTACGACCAG AAGAAATTCC TGAAGGGGTT
  301 TCGCTGGCTC CTTCTGAGGA GCCAGCTCTA CAGGCAGCTC AGAAGACTTT
  351 AGCTCAGCTG CCTAAGGAAT TGGATCAGTT AGATACAGAT ATTCAGGAAG
  401 TGTTCGCATG TTTAAGAAAG CTGAAAGATT CTAAGTATGA AAGTCGAAGT
  451 TTTTTAAACG ATGCTAAGAA GGAGCTTCGA GTTTTTGACT TTGTGGTTGA
  501 GGATACCCTC TCGGAGATTT TCGAGTTGCG GCAGATTGTG GCTCAAGAGG
  551 GATGGGATTT AAACTTTTTG ATCAATGGGG GACGAAGCCT CATGATGACT
  601 GCAGAATCTG AATCGCTTGA TTTGTTTCAT GTATCGAAGC GGCTAGGGTA
  651 TTTACCTTCT GGGGATGTTC GAGGGGAGGG GTTAAAGAAA TCTGCGAAGG
  701 AGATAGTCGC TCGTTTGATG AGCTTGCATT GCGAGATTCA CAAGGTGGCG
  751 GTAGCGTTTG ATAGGAATTC CTATGCGATG GCAGAAAAGG CGTTTGCGAA
  801 AGCGTTGGGA GCCTTAGAAG AGAGTGTGTA TCGGAGTCTG ACGCAGAGTT
  851 ATAGAGATAA ATTTTTGGAG AGCGAGAGGG CGAAGATCCC ATGGAATGGG
  901 CATATAACCT GGTTAAGAGA TGATGCGAAG AGTGGGTGTG CTGAAAAGAA
  951 GCTTCGGGAT GCCGAGGAAC GTTGGAAGAA ATTTAGGAAA GCAGTCTTTT
1001 GGGTAGAAGA AGACGGGGGC TTTGACATCA ATAATCTCCT TGGAGACTGG
1051 GGGACAGTGC TTGATCCTTA TAGACAAGAG AGAATGGACG AGATAACGTT
1101 CCATGAGTTG TATGAAAAAA CTACGTTTTT GAAAAGACTG CACAGAAAGT
1151 GTGCGTTAGC GAAAACAACC TTTGAAAAGA AGAGATCTAA AAAGAATTTG
1201 CAGGCAGTCG AGGAGGCGAA TGCACGTAGG TTGAAATATG TAAGGGATTG
1251 GTATGATCAG GAGTTTCAGA AAGCAGGGGA GAGATTAGAG AAACTGCATG
1301 CTTTGTATCC TGAGGTCTCA GTCTCTATAA GAGAGAACAA AATACAAGAG
1351 ACGCGCTCTA ATTTAGAGAA AGCCTATGAG GCTATCGAAG AGAACTATCG
1401 TTGCTGTGTC CGAGAGCAAG AGGACTACTG GAAAGAAGAA GAGAAAAGGG
1451 AAGCGGAGTT TAGGGAGAGG GGAAACAAGA TTCTTTCTCC TGAGGAGCTG
1501 GAAAGTTCTT TGGAGCAATT CGACCATGGT TTGAAAAATT TTTCTGAGAA
1551 ATTAATGGAA TTGGAAGGGC ATATCTTAAA ACTTCAGAAA GAAGCCACAG
1601 CAGAGGTGGA GAATAAAATA CTTTCAGATG CAGAGAGCCG CCTTGAGATT
1651 GTATTTGAAG ATGTCAAGGA GATGCCCTGT CGAATTGAGG AGATAGAGAA
1701 GACGCTGCGT ATGGCGGAGC TGCCCCTACT TCCTACGAAG AAGGCGTTTG
1751 AGAAGGCCTG CTCACAATAT AATAGCTGCG CAGAGATGTT GGAGAAGGTG
1801 AAGCCTTACT GCAAGGAGAG CCTCGCCTAT GTGACTAGCA AAGAGCGTTT
1851 AGTGAGCTTG GATGAAGATT TACGACGAGC CTACACAGAG TGTCAGAAGA
1901 GATTCCAGGG GGATTCGGGT TTGGAGTCGG AAGTAAGAGC CTGTCGAGAG
1951 CAACTGCGAG AGCGGATCCA AGAGTTTGAA ACTCAAGGGC TGGACTTGGT
2001 GGAAAAAGAG TTGCTTTGTG TGAGTAGTAG ATTAAGAAAT ACAGAGTGCG
2051 ATTGTGTATC TGGTGTTAAG AAAGAAGCAC CTCCTGGTAA GAAGTTTTAT
2101 GCCCAGTATT ATGATGAGAT TTATCGAGTT AGAGTTCAAT CCCGATGGAT
2151 GACGATGTCT GAGAGATTGA GAGAGGGAGT TCAAGCATGC AACAAGATGT
2201 TGAAGGCAGG CCTAAGCGAA GAAGATAAGG TTCTTAAAGA AGAAGAGTAT
2251 TGGTTGTATC GAGAGGAGAG AAAGAATAAA GAGAAACGTT TGGTTGGTAC
2301 TAAGATAGTA GCAACGCAGC AGCGAGTTGC AGCATTTGAA TCCATAGAAG
2351 TTCCTGAGAT TCCTGAGGCC CCAGAGGAGA AACCGAGTTT GCTGGATAAA
2401 GCGCGTTCTT TATTTACTCG CGAGGACCAT ACCTAG
```

The PSORT algorithm predicts inner membrane (0.461).

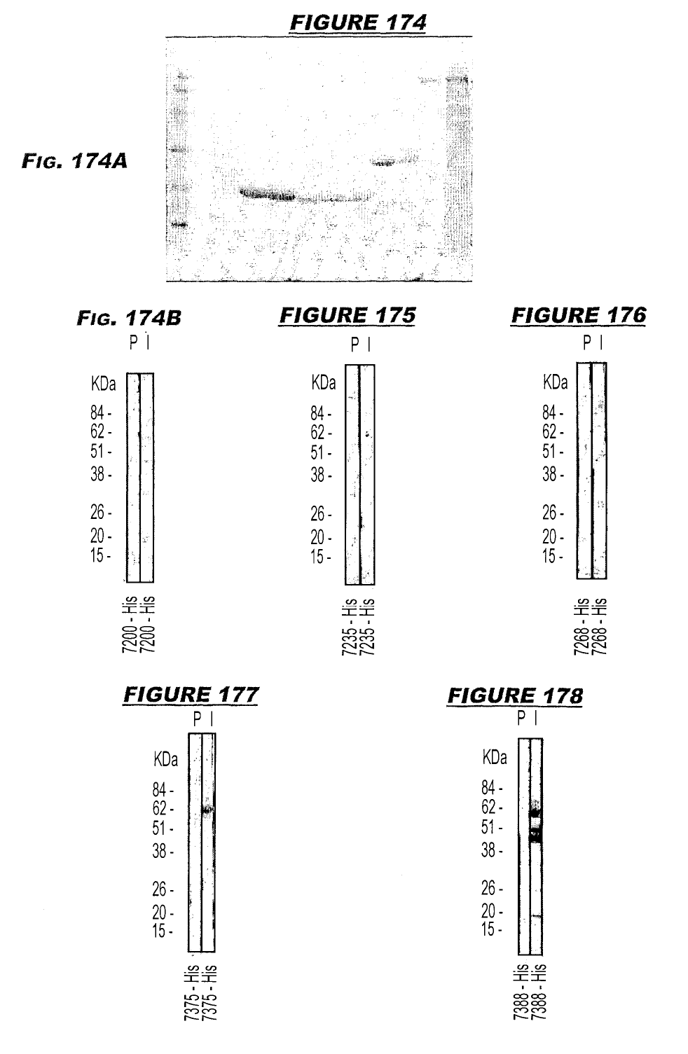

The proteins were expressed in E. coli and purified as his-tag products (FIG. 174: 7200=lanes 2-3; 7236=lanes 4-5; 7268=lanes 6-8; 7375=lanes 9-10; 7388=lanes 11-12). The recombinant proteins were used to immunise mice, whose sera were used in Western blots (FIGS. 174, 175, 176, 177 & 178) and for FACS analysis.

These experiments show that cp7200, cp7235, cp7268, cp7375 & cp7388 are surface-exposed and immunoaccessible proteins and that they are useful immunogens. These properties are not evident from the sequence alone.

### Example 179

The following C. pneumoniae protein (PID 4376723) was expressed <SEQ ID 357; cp6723>:

```
    1 MATSVAPSPV PESSPLSHAT EVLNLPNAYI TQPEPIPAAP WETFRSKLST
  51 KHTLCFALTL LLTLGGTISA GYAGYTGNWI ICGIGLGIIV LTLILALLLA
101 IPLKNKQTGT KLTDEISQDI SSIGSGFVQR YGLMFSTIKS VHLPELTTQN
151 QEKTRILNEI EAKKESIQNL ELKITECQNK LAQKQPKRKS SQKSFMRSIK
201 HLSKNPVILF DC*
```

The cp6723 nucleotide sequence <SEQ ID 358> is:

```
    1 ATGGCAACTT CCGTAGCCCC ATCACCAGTC CCCGAGAGCA GCCCTCTCTC
  51 TCATGCTACA GAAGTTCTCA ATCTTCCTAA TGCTTATATT ACGCAGCCTC
101 ATCCGATTCC AGCGGCTCCT TGGGAGACCT TTCGCTCCAA ACTTTCCACA
151 AAGCATACGC TCTGTTTTGC CTTAACACTA CTGTTAACCT TAGGGGGAAC
201 GATCTCAGCA GGTTACGCAG GATATACTGG AAACTGGATC ATCTGTGGCA
251 TCGGCTTGGG AATTATCGTA CTCACACTGA TTCTTGCTCT TCTTCTAGCA
301 ATCCCTCTTA AAAATAAGCA GACAGGAACA AAACTGATTG ATGAGATATC
351 TCAAGACATT TCCTCTATAG GATCAGGATT TGTTCAGAGA TACGGGTTGA
401 TGTTCTCTAC AATTAAAAGC GTGCATCTTC CAGAGCTGAC AACACAAAAT
451 CAAGAAAAAA CAAGAATTTT AAATGAAATT GAAGCGAAAA AGGAATCGAT
501 CCAAAATCTT GAGCTTAAAA TTACTGAGTG CCAAAACAAG TTAGCACAGA
551 AACAGCCGAA ACGGAAATCA TCTCAGAAAT CATTTATGCG TAGTATTAAG
601 CACCTCTCCA AGAACCCTGT AATTTTGTTC GATTGCTGA
```

The PSORT algorithm predicts inner membrane (0.6095).

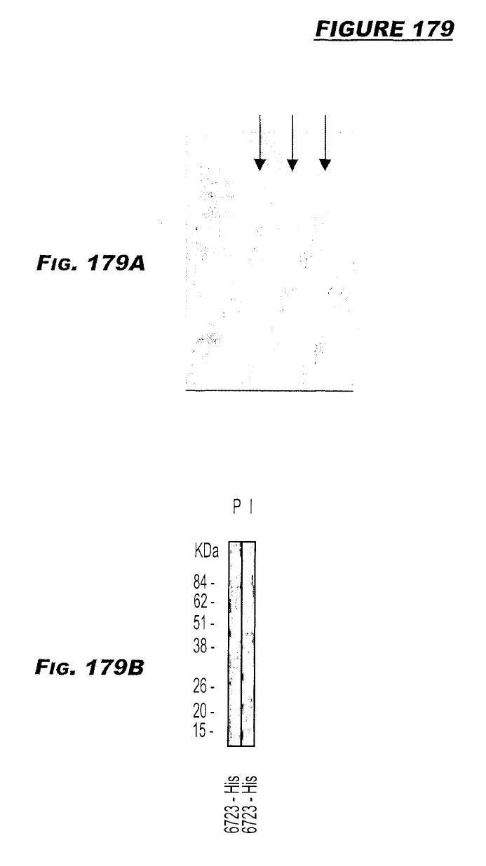

The protein was expressed in E. coli and purified as a his-tag product (FIG. 179A). The recombinant protein was used to immunise mice, whose sera were used in a Western blot (FIG. 179B) and for FACS analysis.

These experiments show that cp6723 is a surface-exposed and immunoaccessible protein, and that it is a useful immunogen. These properties are not evident from the sequence alone.

### Example 180

The following C. pneumoniae protein (PID 4376749) was expressed <SEQ ID 359; cp6749>:

```
    1 MSYYFSLWYL KVQQHFQAAF DFTRSLCSRI SNFALGVIAL LPIIGQLYVG
  51 LDWLLSRIKK PEFPSDVDQI VRVEHVVGHD HRSRVEDILK RQRLSLEPRD
101 EGKVHGDLPS APFF*
```

The cp6749 nucleotide sequence <SEQ ID 360> is:

```
    1 ATGAGTTATT ACTTTTCTCT TTGGTATCTG AAGGTGCAAC AGCACTTTCA
  51 AGCAGCATTT GATTTTACTC GCTCCCTGTG TTCACGAATT TCTAATTTTG
101 CTTTGGGAGT GATTGCATTG CTTCCTATTA TTGGGCAGTT GTATGTAGGG
151 CTGGACTGGC TCCTCTCTAG GATAAAAAAG CCAGAATTTC CTTCCGATGT
201 GGATCAGATC GTGCGAGTAG AACACGTCGT GGGTCACGAC CATAGAAGTC
251 GAGTTGAAGA TATTCTAAAG AGACAAAGGC TCTCATTAGA GCCTAGAGAC
301 GAGGGGAAGG TTCACGGAGA TCTGCCTTCA GCTCCTTTTT TTTGA
```

The PSORT algorithm predicts inner membrane (0.2996).

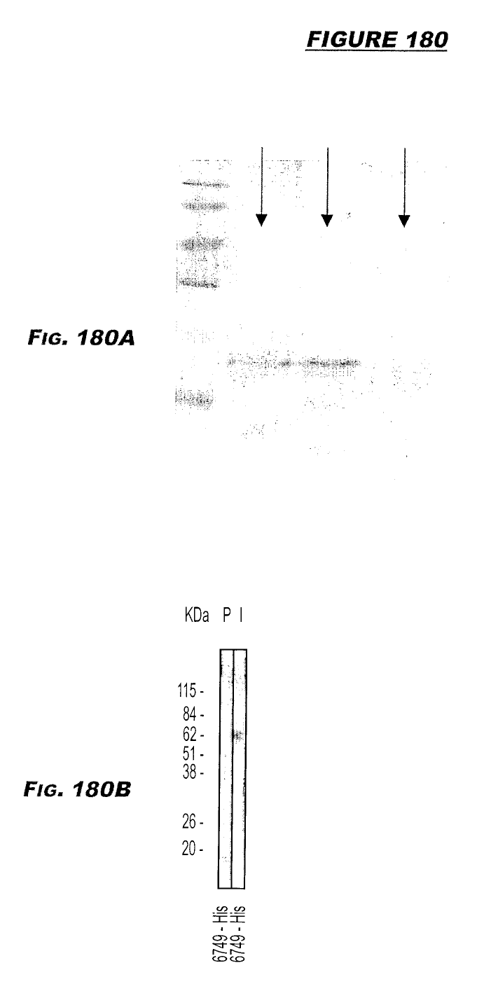

The protein was expressed in E. coli and purified as a his-tag product (FIG. 180A). The recombinant protein was used to immunise mice, whose sera were used in a Western blot (FIG. 180B) and for FACS analysis.

These experiments show that cp6749 is a surface-exposed and immunoaccessible protein, and that it is a useful immunogen. These properties are not evident from the sequence alone.

### Example 181, Example 182, Example 183, Example 184 and Example 185

The following C. pneumoniae protein (PID 4376301) was expressed <SEQ ID 361; cp6301>:

```
    1 LNQDLQNVYQ ECQKATGLES EVSAYRDHLR EQITEFETQG LDVIKEELLF
  51 VSSTLKSKLS YDPLIADIPC MKFYEEYYDG IDKARVQSRW LEKSERYRKA
101 KKGFQEMLKE GLFKEDQALK KAEYRLLREK RMNKEKLLIC NKIEAAQQRV
151 QEFGPSDS*
```

The cp6301 nucleotide sequence <SEQ ID 362> is:

```
    1 TTGAATCAGG ATTTACAAAA TGTATACCAA GAGTGCCAGA AGGCTACAGG
  51 TTTAGAATCG GAAGTGAGTG CATATAGAGA TCATCTTAGA GACCAGATCA
101 CAGAGTTTGA AACTCAAGGG CTGGACGTGA TAAAAGAAGA ACTTCTTTTT
151 GTGAGTAGTA CTCTCAAAAG TAAATTGAGC TATGATCCAT TAATAGCAGA
201 CATTCCCTGT ATGAAGTTTT ATGAGGAGTA TTATGATGGC ATTGATAAAG
251 CGAGAGTTCA ATCCCGATGG CTGGAGAAGT CTGAGAGGTA TAGAAAGGCG
301 AAGAAGGGAT TCCAAGAGAT GCTGAAGGAA GGCCTATTCA AAGAAGATCA
351 GGCTTTGAAA AAAGCAGAGT ATAGATTACT TCGAGAGAAG AGAATGAATA
401 AGGAGAAGCT TTTGATTTGC AATAAGATAG AAGCAGCTCA GCAGCGAGTC
451 CAAGAATTTG GACCCTCGGA TTCATAA
```

The PSORT algorithm predicts cytoplasm (0.4621).

The following C. pneumoniae protein (PID 4376558) was also expressed <SEQ ID 363; cp6558>:

```
    1 MNIPAPQVPV IDEPVVNNTS SYGLSLKSSL RPITYLILAI LAIATLMSVL
  51 YFCGIISVGT FVLGMLIPLS VCSVLCVAYL FYQQSSIEKT KVFSITSPSV
101 FFSDEDLNLL LGREEDSVSA IDELLKNFPA DDFRRPKMLP YSNFLDEQGR
151 PNESREEDSH TSKIL*
```

The cp6558 nucleotide sequence <SEQ ID 364> is:

```
    1 ATGAACATAC CCGCTCCCCA AGTACCAGTC ATAGATGAGC CTGTAGTGAA
  51 CAACACAAGT AGCTATGGTC TTTCATTGAA AAGTAGTTTA AGACCGATTA
101 CTTATTTGAT TTTAGCTATC TTAGCTATAG CCACACTGAT GTCTGTTCTC
151 TACTTTTGTG GCATCATTAG TGTTGGGACG TTTGTTTTGG GCATGCTGAT
201 CCCTCTATCG GTCTGCTCTG TTCTTTGCGT TGCCTATTTA TTCTATCAGC
251 AATCTTCTAT AGAAAAGACT AAGGTCTTTT CTATAACCAG TCCTTCAGTA
301 TTTTTCTCTG ATGAGGATCT TAATTTACTC TTAGGTCGAG AAGAAGATTC
351 AGTGTCTGCA ATTGATGAAC TTCTTAAGAA CTTTCCAGCT GATGATTTCC
401 GTAGGCCGAA GATGCTTCCT TATTCAAATT TTCTAGATGA GCAGGGAAGG
451 CCTAATGAGA GTAGGGAAGA AGACTCTCAT ACTTCCAAGA TCTTATAA
```

The PSORT algorithm predicts inner membrane (0.4630).

The following C. pneumoniae protein (PID 4376630) was also expressed <SEQ ID 365; cp6630>:

```
    1 MSMTIVPHAL FKNHCECHST FPLSSRTIVR IAIASLFCIG ALAALGCLAP
  51 PVSYIVGSVL AFIAFVILSL VILALIFGEK KLPPTPRIIP DRFTHVIDEA
101 YGLSISAFVR EQQVTLAEFR QFSTALLCNI SPEEKIKQLP SELRSKVESF
151 GISRLAGDLE KNNWPIFEDL LSQTCPLYWL QKFISAGDPQ VCRDLGVPRE
201 CYGYYWLGPL GYSTAKATIF CKETHHILQQ LTKEDVLLLK NKALQEKWDT
251 DEVKAIVERI YTTYTARGTL KTEAGGLTKE TISKELLLLS LHGYSFDQLQ
301 LITQLPRDAW DWLCFVDNST AYNLQLCALV GALSSQNLLD ESSIDFDVNL
351 GLYVIQDLKE AVQAFSASDE RKKELGKFLL RHLSSVSKRL ESVLRQGLHR
401 IALEHGNARA RVYDVNFVTG ARIHRKTSIF FKD*
```

The cp6630 nucleotide sequence <SEQ ID 366> is:

```
      1 ATGAGCATGA CGATCGTTCC ACATGCTTTA TTTAAAAATC ATTGCGAGTG
    51 TCATTCTACC TTTCCTTTGA GTTCAAGGAC TATTGTAAGA ATAGCCATTG
  101 CCAGCCTCTT TTGTATAGGT GCATTAGCAG CTTTAGGCTG TTTGGCTCCT
  151 CCCGTTTCTT ATATTGTTGG GAGTGTTTTA GCTTTTATTG CCTTTGTCAT
  201 TCTTTCTTTA GTAATTTTAG CTTTGATTTT TGGAGAGAAG AAGCTTCCAC
  251 CAACACCAAG AATCATTCCT GATAGATTTA CTCACGTGAT AGATGAAGCT
  301 TATGGCCTTT CAATCTCTGC ATTTGTAAGA GAACAGCAGG TAACATTAGC
  351 CGAGTTTAGA CAATTTTCTA CTGCCCTGTT GTGTAACATA TCTCCTGAAG
  401 AGAAAATCAA ACAATTGCCT TCTGAATTGC GAAGTAAAGT AGAGAGTTTT
  451 GGTATTAGCA GGCTCGCAGG TGATTTAGAA AAGAATAATT GGCCAATATT
  501 TGAAGATCTT TTAAGCCAAA CCTGCCCGTT ATATTGGCTT CAGAAATTTA
  551 TATCAGCAGG AGATCCACAA GTTTGTAGAG ACCTAGGTGT CCCTAGAGAA
  601 TGTTATGGGT ACTATTGGCT AGGGCCTTTG GGATACAGTA CAGCTAAGGC
  651 TACAATTTTT TGTAAAGAGA CGCATCATAT TCTTCAACAA TTAACGAAAG
  701 AGGACGTTCT TTTATTAAAA AACAAGGCTC TTCAAGAGAA ATGGGATACT
  751 GATGAAGTCA AAGCAATTGT AGAGCGTATC TACACTACCT ATACGGCACG
  801 AGGAACTCTA AAGACCGAAG CAGGGGGACT TACAAAAGAG ACAATCAGTA
  851 AGGAATTGCT ATTGTTGAGC TTGCATGGCT ATTCTTTTGA TCAGCTACAG
  901 CTGATCACTC AACTTCCTAG AGATGCTTGG GATTGGCTGT GTTTTGTAGA
  951 TAACAGTACC GCATACAACC TTCAGCTTTG TGCTCTTGTA GGAGCTTTGT
1001 CATCCCAAAA TCTTCTTGAC GAATCTTCTA TCGATTTTGA TGTAAACCTA
1051 GGCCTGTATG TGATTCAGGA TCTAAAAGAA GCTGTTCAAG CATTTTCTGC
1101 TTCTGATGAG CCAAAGAAAG AACTAGGTAA ATTCTTGTTA AGGCATTTGA
1151 GTTCAGTTTC TAAGCGATTA GAGAGTGTAT TAAGACAGGG TCTTCACAGA
1201 ATAGCTCTAG AGCATGGAAA TGCCAGAGCT AGGGTTTATG ACGTCAATTT
1251 TGTAACAGGA GCTAGAATTC ATAGGAAGAC GAGTATCTTC TTTAAAGACT
1301 AA
```

The PSORT algorithm predicts inner membrane (0.7092).

The following C. pneumoniae protein (PID 4376633) was also expressed <SEQ ID 367; cp6633>:

```
    1 MVNIQPVYRN TQVNYSQATQ FSVCQPALSL IIVSVVAAVL AIVALVCSQS
  51 LLSIELGTAL VLVSIILFAS AMFMIYKMRQ EPKELLIPKK IMELIQEHYP
101 SIVVDFIRDQ EVSIYEIHHL ISILNKTNVF DKAPVYLQEK LLQFGIEKFK
151 DVHPSKLPNF EEILLQHCPL HWLGRLVYPM VSDVTPGTYG YYWCGPLGLY
201 ENAPSLFERR SLLLLKKISF GEFALLEDGL KKNTWSSSEL VQIRQNLFTR
251 YYADKEEVDE AELNADYEQF DSLLHLIFSH KLS*
```

The cp6633 nucleotide sequence <SEQ ID 368> is:

```
    1 ATGGTTAATA TACAGCCTGT GTATAGGAAT ACCCAAGTCA ACTATAGTCA
  51 GGCTACCCAA TTTTCGGTGT GCCAGCCAGC GCTTAGCCTG ATTATCGTTT
101 CTGTTGTTGC TGCTGTACTC GCTATTGTAG CTTTGGTATG CAGTCAATCT
151 CTTTTATCCA TAGAGTTAGG AACTGCTCTT GTTCTAGTTT CTCTTATTCT
201 TTTTGCTTCT GCTATGTTTA TGATTTATAA GATGAGACAA GAACCTAAGG
251 AGTTGCTGAT CCCTAAGAAA ATCATGGAAC TCATCCAAGA ACATTATCCA
301 AGTATTGTTG TTGATTTTAT TAGAGATCAG GAGGTTTCCA TTTATGAGAT
351 ACATCACTTG ATCTCTATTC TTAATAAGAC GAATGTTTTC GACAAAGCAC
401 CAGTATATTT ACAAGAAAAA CTCTTACAGT TTGGCATTGA GAAGTTCAAA
451 GATGTACATC CAAGTAAGCT CCCTAATTTT GAAGAAATTC TTCTACAGCA
501 TTGCCCATTG CATTGGTTGG GACGTCTGGT ATATCCCATG GTATCGGATG
551 TCACTCCAGG AACCTATGGA TACTATTGGT GTGGTCCTTT AGGACTGTAC
601 GAGAACGCTC CCTCTCTTTT TGAACGTCGA TCTCTTCTAT TGTTAAAGAA
651 AATTAGCTTT GGAGAGTTTG CTCTTTTAGA AGATGGTCTC AAGAAAAACA
701 CGTGGAGTTC TTCGGAACTC GTTCAAATCA GACAAAACCT TTTTACAAGA
751 TATTATGCTG ATAAAGAAGA GGTAGATGAA GCAGAGTTAA ACGCTGATTA
801 CGAACAGTTT GATTCCCTCC TTCACCTTAT TTTTTCTCAC AAGCTCTCTT
851 GA
```

The PSORT algorithm predicts inner membrane (0.7283).

The following C. pneumoniae protein (PID 4376642) was also expressed <SEQ ID 369; cp6642>:

```
    1 MATISPISLT VDHPLVDTKK KSCSNFDKIQ SRILLITAIF AVLVTIGTLL
  51 IGLLLNIPVI YFLTGISFIA VVLSNFILYK RATTLLKPRA CGKHKEIKPK
101 RVSTNLQYSS ISIAINRSKE NWEHQPKDLQ NLPAPSALLT DNPYFIWKAK
151 HSLFSLVSLL PGGNPEHLLI SASENLGKTL LIEETSQNAP ISSYVDTTPS
201 PKSLLNEAIQ ETRVEINTEL PAGDSGERLY WQPDFRGRVF LPQIPTTPEA
251 IYQYYYALYV TYIQTAINTN TQIIQIPLYS LREHLYSREL PPQSRMQQSL
301 AMITAVKYMA ELHPEYPLTI ACVERSLAQL PQESIEDLS*
```

The cp6642 nucleotide sequence <SEQ ID 370> is:

```
      1 ATGGCTACAA TCTCACCCAT ATCTTTAACT GTAGATCATC CCCTAGTAGA
    51 CACTAAAAAA AAATCCTGCA GCAACTTTGA TAAGATTCAG TCTCGAATTC
  101 TATTGATTAC TGCAATCTTT GCTGTCTTAG TTACTATAGG GACCCTACTT
  151 ATTGGTTTGC TTTTAAATAT TCCTGTTATC TATTTCCTCA CAGGAATTTC
  201 ATTTATTGCT GTTGTTCTTA GCAACTTTAT CCTTTATAAA CGAGCAACCA
  251 CCCTCTTAAA ACCGCGTGCT TGTGGCAAAC ACAAAGAAAT AAAACCAAAA
  301 AGGGTCTCCA CCAACCTACA GTATTCTTCT ATCTCTATCG CAATCAATCG
  351 TTCTAAAGAA AACTGGGAAC ACCAACCCAA GGACCTACAG AATCTCCCCG
  401 CACCCTCTGC ATTACTCACA GATAACCCTT ACGAGATATG GAAAGCTAAA
  451 CATTCACTGT TTTCCCTAGT ATCCCTCCTA CCGGGAGGCA ATCCAGAACA
  501 TCTCTTAATT TCAGCTTCCG AAAATTTAGG AAAGACTCTG TTAATTGAAG
  551 AAACCTCGCA AAATGCGCCT ATATCCTCCT ACGTAGATAC CACTCCCTCC
  601 CCAAAATCCT TGCTCAATGA GGCAATTCAG GAAACCAGGG TAGAAATAAA
  651 TACAGAACTC CCTGCGGGAG ATTCAGGAGA ACGTTTATAC TGGCAACCCG
  701 ATTTCCGAGG CCGCGTCTTC CTCCCACAAA TACCAACAAC TCCTGAAGCC
  751 ATCTACCAAT ACTACTATGC ACTCTATGTC ACTTATATCC AGACTGCGAT
  801 CAATACGAAC ACCCAAATTA TCCAAATCCC TTTATACAGC TTGAGGGAGC
  851 ATCTCTATTC TAGAGAATTG CCCCCGCAAT CAAGAATGCA ACAATCTTTG
  901 GCTATGATTA CAGCAGTAAA ATACATGGCC GAGCTGCACC CAGAATATCC
  951 GCTAACTATT GCTTGTGTTG AAAGATCCTT AGCCCAACTA CCTCAAGAAA
1001 GTATTGAGGA TCTCTCTTAG
```

The PSORT algorithm predicts inner membrane (0.5288).

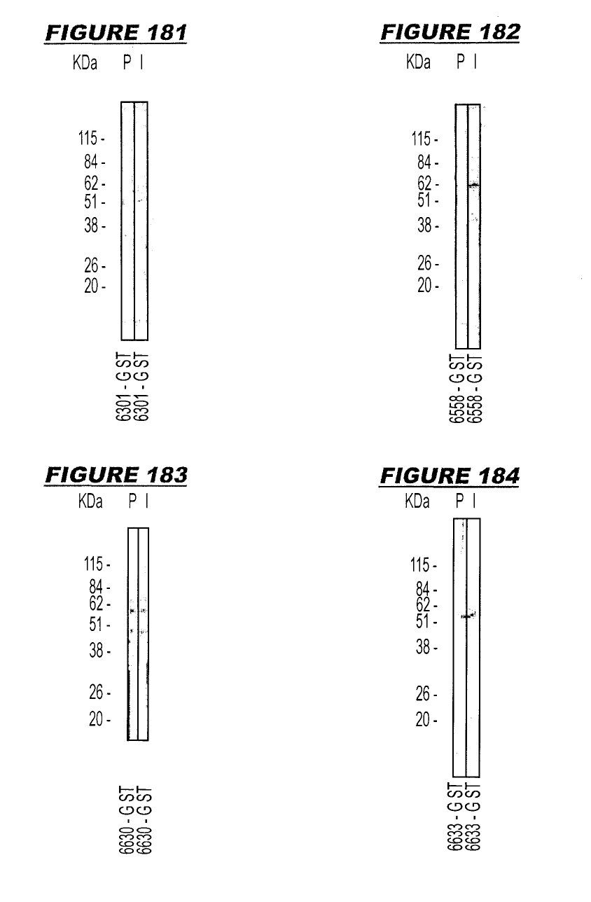

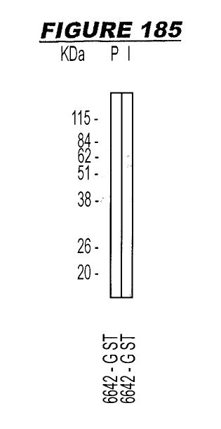

The proteins were expressed in E. coli and purified as GST-fusion products. The recombinant proteins were used to immunise mice, whose sera were used in Western blots (FIGS. 181-185) and for FACS analysis.

These experiments show that cp6301, cp6558, cp6630, cp6633 and cp6642 are surface-exposed and immunoaccessible proteins, and that they are useful immunogens. These properties are not evident from their sequences alone.

### Example 186

The following C. pneumoniae protein (PID 4376389) was expressed <SEQ ID 371; cp6389>:

```
    1 MSEVKPLFLK NDSFDLATQR FQNLINMLQE QAEIYNEYEE KNARVQNEIK
  51 EQKDFVKRCI EDFEARGLGV LKEELASLTR DFHDKAKAET SMLIECPCIG
101 FYYSIHQEEQ RQRQERLQKM AERYRDCKQV LEAVQVEQKD MISSRVVVDD
151 SYFEEEKEEQ KVDNRKKEQD *
```

The cp6389 nucleotide sequence <SEQ ID 372> is:

```
    1 ATGTCAGAAG TGAAGCCTTT GTTTTTAAAG AATGACTCTT TTGATTTGGC
  51 AACTCAGAGA TTCCAGAATC TAATTAACAT GCTACAAGAG CAAGCCGAGA
101 TATATAACGA GTATGAAGAA AAGAATGCTA GGGTTCAGAA TGAGATTAAG
151 GAGCAAAAGG ACTTTGTGAA AAGATGCATA GAGGACTTTG AAGCCAGAGG
201 ACTGGGGGTG CTAAAAGAAG AGCTTGCATC TTTGACGCGT GATTTCCATG
251 ATAAAGCAAA AGCAGAGACT TCTATGCTCA TTGAATGTCC TTGTATTGGT
301 TTTTATTATA GTATTCATCA GGAGGAACAA AGGCAAAGGC AAGAAAGGCT
351 TCAAAAGATG GCTGAGCGCT ATAGGGACTG TAAACAAGTC TTGGAGGCTG
401 TCCAGGTGGA GCAAAAAGAT ATGATATCTT CTAGAGTCGT TGTCGATGAC
451 AGCTACTTTG AAGAAGAAAA AGAAGAACAA AAGGTGGATA ACAGAAAGAA
501 AGAACAGGAC TAG
```

The PSORT algorithm predicts cytoplasm (0.3193).

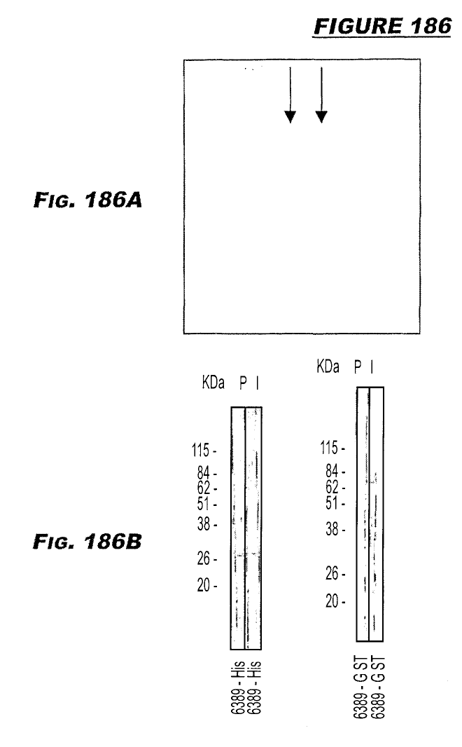

The protein was expressed in E. coli and purified as a GST-fusion product (FIG. 186A) and also in his-tagged form. The recombinant proteins were used to immunise mice, whose sera were used in a Western blot (FIG. 186B) and for FACS analysis.

These experiments show that cp6389 is a surface-exposed and immunoaccessible protein, and that it is a useful immunogen. These properties are not evident from the sequence alone.

### Example 187

The following C. pneumoniae protein (PID 4376792) was expressed <SEQ ID 373; cp6792>:

```
    1 VLQEHFFLSE DVITLAQQLL GHKLITTHEG LITSGYIVET EAYRGPDDKA
  51 CHAYNYRKTQ RNRAMYLKGG SAYLYRCYGM HHLLNVVTGP EDIPHAVLIR
101 AILPDQGKEL MIQRRQWRDK PPHLLTNGPG KVCQALGISL ENNRQRLNTP
151 ALYISKEKIS GTLTATARIG IDYAQEYRDV PWRFLLSPED SGKVLS*
```

The cp6792 nucleotide sequence <SEQ ID 374> is:

```
    1 GTGCTACAAG AACATTTTTT TCTATCGGAA GATGTAATTA CAGTAGCGCA
  51 ACAGCTTTTA GGACATAAAC TCATCACAAC ACATGAGGGT CTGATAACTT
101 CAGGTTACAT TGTAGAAACC GAAGCGTATC GTGGCCCTGA TGACAAAGCA
151 TGCCACGCCT ACAACTACAG AAAAACTCAG AGGAACAGAG CGATGTACCT
201 GAAAGGAGGC TCTGCTTACC TCTACCGTTG CTATGGCATG CATCACCTAT
251 TGAATGTTGT CACTGGACCT GAGGACATTC CCCATGCCGT CCTGATCCGG
301 GCCATCCTTC CTGATCAAGG CAAAGAACTT ATGATCCAAC GCCGCCAATG
351 GAGAGATAAA CCCCCACACC TTCTCACCAA TGGACCCGGA AAAGTGTGCC
401 AAGCTCTAGG AATCTCTTTG GAAAACAATA GGCAACGCCT AAATACCCCA
451 GCTCTCTATA TCAGCAAAGA AAAAATCTCT GGGACTCTAA CAGCAACTGC
501 CCGGATCGGC ATCGATTATG CTCAAGAGTA TCGTGATGTC CCATGGAGAT
551 TTCTCCTATC CCCAGAAGAT TCGGGAAAAG TTTTATCTTA A
```

The PSORT algorithm predicts cytoplasm (0.180).

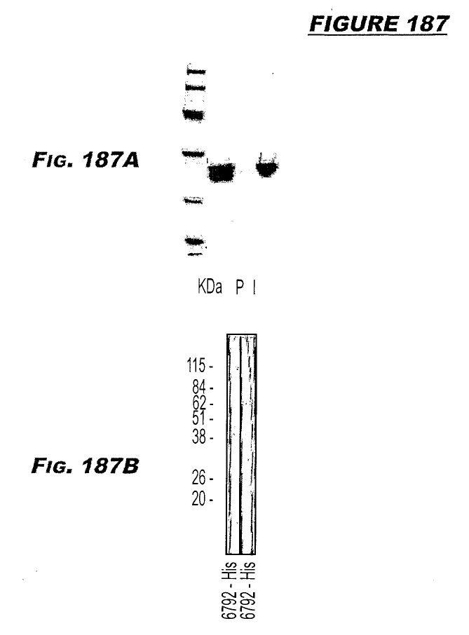

The protein was expressed in E. coli and purified as a his-tagged product (FIG. 187A; lanes 2-4). The recombinant protein was used to immunise mice, whose sera were used in a Western blot (FIG. 187B) and for FACS analysis.

These experiments show that cp6792 is a surface-exposed and immunoaccessible protein, and that it is a useful immunogen. These properties are not evident from the sequence alone.

### Example 188

The following C. pneumoniae protein (PID 4376868) was expressed <SEQ ID 375; cp6868>:

```
    1 MVETVLHNFQ RYLSKYLYRV FRFPCRKKTF LSSERVLARP SFPVDYCPGK
  51 IYDLQEIYEE LNAQLFQGAL RLQIGWFGRK ATRKGKSVVL GLFHENEQLI
101 RIHRSLDRQE IPRFFMEYLV YHEMVHSVVP REYSLSGRSI FHGKKFKEYE
151 QRFPLYDRAV AWEKANAYLL RGYKKRVGGG YGRA*
```

The cp6868 nucleotide sequence <SEQ ID 376> is:

```
    1 ATGGTTGAAA CAGTACTTCA TAATTTCCAA CGTTATCTGA GCAAGTATCT
  51 CTATAGGGTA TTTCGCTTCC CATGTCGTAA AAAGACGTTC CTATCTTCGC
101 ACAGGGTTCT TGCTCGTCCT TCATTCCCAG TAGACTACTG TCCGGGAAAG
151 ATCTATGATT TGCAGGAGAT CTATGAGGAA TTGAATGCGC AGTTATTTCA
201 AGGTGCACTG CGTTTACAGA TTGGTTGGTT CGGAAGGAAA GCTACCAGAA
251 AAGGCAAGAG TGTTGTCTTG GGATTGTTTC ATGAAAATGA ACAGTTAATT
301 CGAATTCATC GTTCTTTAGA TCGGCAGGAA ATCCCAAGAT TTTTTATGGA
351 ATATCTTGTG TATCATGAAA TGGTTCATAG TGTAGTCCCT AGAGAGTATT
401 CTCTATCGGG GCGTTCGATT TTTCATGGTA AAAAGTTTAA AGAATACGAA
451 CAACGTTTCC CCTTGTATGA TCGTGCTGTT GCTTGGGAAA AGGCAAACGC
501 TTATTTATTG CGAGGGTATA AAAAAAGAGT AGGTGGAGGA TATGGCAGGG
551 CATAG
```

The PSORT algorithm predicts bacterial cytoplasm (0.325).

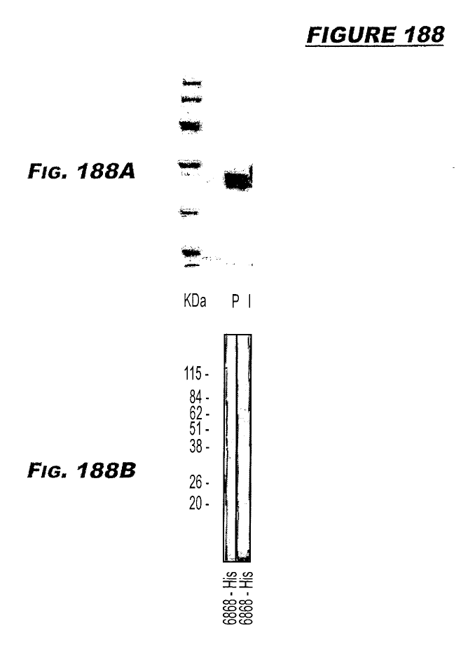

The protein was expressed in E. coli and purified as a his-tag product (FIG. 188A; lanes 2-3). The recombinant protein was used to immunise mice, whose sera were used in a Western blot (FIG. 188B) and for FACS analysis.

These experiments show that cp6868 is a surface-exposed and immunoaccessible protein, and that it is a useful immunogen. These properties are not evident from the sequence alone.

### Example 189

The following C. pneumoniae protein (PID 4376894) was expressed <SEQ ID 377; cp6894>:

```
    1 MYKRCVLDKI LKGIVAGSLI LLYWSSDLLE RDIKSIKGNV RDIQEDIREI
  51 SRVVKQQQTS QAIPAAPGVM LAPKLVRDEA FALLFGDPSY PNLLSLDPYK
101 QQTLPELLGT NFHPHGILRT AHVGKPENLS PFNGFDYVVG FYDLCIPSLA
151 SPHVGKYEEF SPDLAVKIEE HLVEDGSGDK EFHIYLRPNV FWRPIDPKAL
201 PKHVQLDEVF QRPHPVTAHD IKFFYDAVMN PYVATMRAVA LRSCYEDVVS
251 VSVENDLKLV VRWKAHTVIN EEGKEERKVL YSAFSNTLSL QPLPRFVYQY
301 FANGEKIIED ENIDTYRTNS IWAQNFTMHW ANNYIVSCGA YYFAGMDDEK
351 IVFSRNPDFY DPLAALIDKR FVYFKESTDS LFQDFKTGKI DISYLPPNQR
401 DNFYSFMKSS AYNKQVAKGG AVRETVSADR AYTYIGWNCF SLFFQSRQVR
451 CAMNMAIDRE RIIEQCLDGQ GYTISGPFAS SSPSYNKQIE GWHYSPEEAA
501 RLLEEEGWID TDGDGIREKV IDGVIVPFRF RLCYYVKSVT AHTIADYVAT
551 ACKEIGIECS LLGLDMADLS QAFDEKNFDA LLMGWCLGIP PEDPRALWHS
601 EGAMEKGSAN VVGFHNEEAD KIIDRLSYEY DLKERNRLYH RFHEIIHEFA
651 PYAFLFSRHC SLLYKDYVKN IFVPTHRTDL IPEAQDETVN VTMVWLEKKE
701 DPCLSTS*
```

The cp6894 nucleotide sequence <SEQ ID 378> is:

```
      1 ATGTATAAAA GATGTGTGCT AGATAAAATT TTAAAGGGGA TTGTCGCCGG
    51 TTCTTTAATT TTGTTATACT GGTCCTCAGA CCTACTTGAA AGAGACATTA
  101 AGTCGATAAA AGGTAACGTA AGAGATATTC AAGAAGACAT TCGTGAAATC
  151 TCACGCGTAG TGAAACAACA GCAGACATCA CAAGCTATCC CTGCGGCACC
  201 TGGGGTGATG CTCGCTCCTA AGCTCGTCAG AGACGAAGCT TTTGCTCTAC
  251 TCTTTGGAGA TCCTAGTTAT CCTAATTTAC TTTCCCTAGA CCCCTATAAA
  301 CAGCAGACTC TTCCTGAACT TCTAGGAACA AATTTCCACC CTCATGGTAT
  351 CCTACGCACT GCCCATGTCG GAAAACCCGA AAATCTGAGC CCTTTTAATG
  401 GCTTTGATTA TGTCGTGGGC TTTTACGATC TCTGTATTCC TAGTTTAGCT
  451 TCTCCCCACG TAGGGAAATA CGAAGAATTT TCTCCAGATC TCGCTGTGAA
  501 AATAGAAGAA CATCTTGTTG AAGATGGTTC TGGGGATAAA GAGTTTCACA
  551 TCTATCTGAG GCCGAATGTT TTTTGGCGTC CTATAGATCC TAAGGCCCTT
  601 CCAAAACACG TTCAGTTAGA CGAAGTATTT CAACGTCCTC ATCCTGTGAC
  651 AGCTCATGAT ATTAAGTTTT TCTACGACGC TGTTATGAAC CCTTATGTAG
  701 CAACCATGCG AGCAGTGGCT CTGCGCTCTT GTTATGAAGA TGTGGTTTCT
  751 GTCTCAGTAG AAAACGATTT AAAATTAGTA GTCAGATGGA AAGCACACAC
  801 GGTAATCAAT GAAGAAGGAA AGGAAGAGCG CAAAGTGCTC TACTCTGCAT
  851 TTTCTAATAC CTTAAGCTTG CAGCCCCTCC CTAGATTTGT ATATCAGTAT
  901 TTTGCTAACG GGGAAAAAAT CATTGAAGAT GAGAATATCG ATACCTACCG
  951 AACCAATTCC ATTTGGGCGC AAAACTTCAC TATGCATTGG GCAAACAACT
1001 ATATTGTAAG TTGTGGAGCC TACTACTTTG CAGGGATGGA TGATGAGAAA
1051 ATCGTGTTTT CTAGAAATCC TGACTTCTAT GATCCTCTTG CGGCTCTTAT
1101 TGACAAGCGT TTCGTCTATT TTAAGGAAAG CACAGACTCC CTATTCCAAG
1151 ATTTTAAGAC AGGGAAAATA GACATCTCTT ACCTTCCACC CAACCAAAGA
1201 GATAATTTCT ATAGTTTTAT GAAAAGCTCC GCTTATAACA AACAGGTAGC
1251 TAAGGGAGGA GCCGTCCGTG AAACAGTCTC AGCAGATCGA GCATATACGT
1301 ACATAGGATG GAATTGCTTT TCATTATTTT TCCAAAGCCG ACAGGTGCGC
1351 TGTGCTATGA ACATGGCAAT CGATAGAGAG AGGATTATCG AACAGTGCTT
1401 GGATGGCCAA GGCTATACGA TTAGTGGGCC TTTTGCTTCG AGTTCTCCTT
1451 CTTATAATAA ACAGATCGAA GGGTGGCATT ATTCTCCAGA AGAAGCAGCT
1501 CGTCTCCTGG AAGAAGAGGG ATGGATAGAT ACCGATGGCG ATGGAATCCG
1551 AGAAAAAGTT ATCGATGGTG TGATTGTCCC GTTCCGTTTC CGTTTATGCT
1601 ATTATGTAAA GAGTGTCACC GCTCATACCA TTGCAGATTA CGTAGCTACT
1651 GCTTGTAAGG AAATCGGAAT CGAGTGTAGC CTTCTAGGAC TAGATATGGC
1701 CGATCTTTCG CAAGCTTTTG ATGAAAAGAA TTTCGATGCT CTTTTAATGG
1751 GATGGTGTTT AGGAATTCCT CCTGAGGATC CTAGGGCTTT ATGGCATTCT
1801 GAAGGGGCTA TGGAAAAGGG TTCAGCGAAT GTTGTAGGTT TCCATAATGA
1851 AGAAGCTGAT AAAATCATAG ACAGACTCAG CTACGAATAC GATCTGAAAG
1901 AACGTAATCG CCTGTACCAC CGTTTCCATG AAATTATTCA TGAGGAAGCT
1951 CCTTATGCTT TCTTGTTCTC ACGACATTGT TCCTTACTTT ATAAGGATTA
2001 TGTAAAAAAT ATTTTCGTAC CTACACATAG AACAGATTTA ATTCCTGAAG
2051 CTCAGGATGA GACTGTCAAC GTAACTATGG TATGGCTTGA GAAGAAGGAG
2101 GATCCGTGCT TAAGTACATC CTAA
```

The PSORT algorithm predicts inner membrane (0.162).

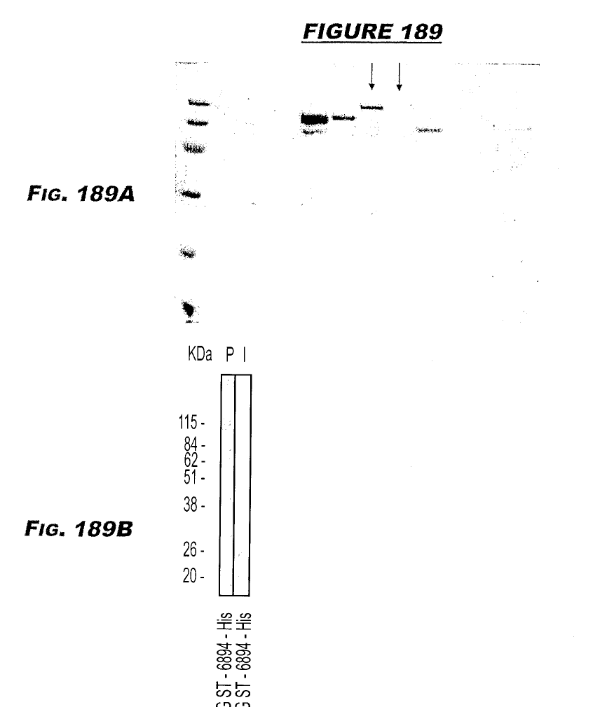

The protein was expressed in E. coli and purified as a his-tag product (FIG. 189A) and also in GST/his form. The recombinant proteins were used to immunise mice, whose sera were used in a Western blot (FIG. 189B) and for FACS analysis.

These experiments show that cp6894 is a surface-exposed and immunoaccessible protein, and that it is a useful immunogen. These properties are not evident from the sequence alone.

### Example 190

The following C. pneumoniae protein (PID 4377193) was identified in the 2D-PAGE experiment <SEQ ID 379; cp7193>:

```
    1 MKRVIYKTIF CGLTLLTSLS SCSLDPKGYN LETKNSRDLN QESVILKENR
  51 ETPSLVKRLS RRSRRLFARR DQTQKDTLQV QANFKTYAEK ISEQDERDLS
101 FVVSSAAEKS SISLALSQGE IKDALYRIRE VHPLALIEAL AENPALIEGM
151 KKMQGRDWIW NLFLTQLSEV FSQAWSQGVI SEEDIAAFAS TLGLDSGTVA
201 SIVQGERWPE LVDIVIT*
```

A predicted leader peptide is underlined.

The cp7193 nucleotide sequence <SEQ ID 380> is:

```
    1 ATGAAAAGAG TCATTTATAA AACCATATTT TGCGGGTTAA CTTTACTTAC
  51 AAGTTTGAGT AGTTGTTCCC TGGATCCTAA AGGATATAAC CTAGAGACAA
101 AAAACTCGAG GGACTTAAAT CAAGAGTCTG TTATACTGAA GGAAAACCGT
151 GAAACACCTT CTCTTGTTAA GAGACTCTCT CGTCGTTCTC GAAGACTCTT
201 CGCTCGACGT GATCAAACTC AGAAGGATAC GCTGCAAGTG CAAGCTAACT
251 TTAAGACCTA CGCAGAAAAG ATTTCAGAGC AGGACGAAAG AGACCTTTCT
301 TTCGTTGTCT CGTCTGCTGC AGAAAAGTCT TCAATTTCGT TAGCTTTGTC
351 TCAGGGTGAA ATTAAGGATG CTTTGTACCG TATCCGAGAA GTCCACCCTC
401 TAGCTTTAAT AGAAGCTCTT GCTGAAAACC CTGCCTTGAT AGAAGGGATG
451 AAAAAGATGC AAGGCCGTGA TTGGATTTGG AATCTTTTCT TAACACAATT
501 AAGTGAAGTA TTTTCTCAAG CTTGGTCTCA AGGGGTTATC TCTGAAGAAG
551 ATATCGCCGC ATTTGCCTCC ACCTTAGGTT TGGACTCCGG GACCGTTGCG
601 TCCATTGTCC AAGGGGAAAG GTGGCCCGAG CTTGTGGATA TAGTGATAAC
651 TTAA
```

The PSORT algorithm predicts periplasmic (0.925).

This shows that cp7193 is an immunoaccessible protein in the EB and that it is a useful immunogen. These properties are not evident from the protein's sequence alone.

It will be appreciated that the invention has been described by way of example only and that modifications may be made whilst remaining within the spirit and scope of the invention.

TABLE II
sequences of the primers used to amplify Cpn genes.

| | | |
|---|---|---|
| Orf ID | N-terminus final primer | C-terminus final primer |

| | | |
|---|---|---|
| CP0014P | GCGTC CCG GGTCATATG AAGTCTTCTTTCCCCA | GCGT CTC GAG ATGAAAGAGTTTTTGCG |
| CP0015P | GCGTCCCGGGTCATATG TCAGCTCTGTTTTCTGA | GCGT CTC GAG GAATTGGTATTTTGCTC |
| CP0016P | GCGTCCCGGGTCATATG GCCGATCTCACATTAG | GCGT CTC GAG GTCCAAGTTAAGGTAGCA |
| CP0017P | GCGT CCG GGTCATATG GGTATCAAGGGAACTG | GCGT CTC GAG AAATCCGAATCTTCC |
| CP0019P | GCGTCCCGGGTCAT ATGCAAGACTCTCAAGACTATAG | GCGT CTC GAG AAATCGGTATTTACCC |
| CP6260P | GCGTC CCG GGT GCTAGCACTACGATTTCTTTAACCC | GCGT CTC GAG AAAACGAAATTTGCTTC |
| CP6397P | GCGTC CCG GGTCATATGTTTAAACTGCTAAAAAATCTATT | GCGT CTC GAG ATGAAAGAAGAGTCCTCG |
| CP6456P | GCGTC CCG GGT CATATG TCATCTCCTGTAAATAACA | GCGT CTC GAG CTGACCATCTCCTGTT |
| CP6466P | GCGTC CCG GGT CAT ATG TGCAAGGAGTCCAGT | GCGT CTC GAG ATTTTCCTTAGCATAACG |
| CP6467P | GCGTC CCG GGT CAT ATG TGTTCCCCATCCCAA | GCGT CTC GAG TAGTTTTTCTATAAAACGAAAGTCT |
| CP6468P | GCGTC CCG GGT CAT ATG TGCTCCTCCTACTCTTC | GCGT CTC GAG GGGGAAATAGGTATATTTGA |
| CP6469P | GCGTC CCG GGT CAT ATG AGCTGCTCAAAGCAA | GCGT CTC GAG ACTTAAGATATCGATATTTTTGA |
| CP6552P | GCGTC CCG GGT CAT ATG TGCCATAAGGAAGATG | GCGT CTC GAG ACCATTGTCTTGAGTCAT |
| CP6567P | GCGTC CCG GGT CAT ATG ACCTCACCGATCCCC | GCGT CTC GAG AGAAGCCGGTAGAGGC |
| CP6576P | GCGTC CCG GGT CAT ATG ACTGAAAAAGTTAAAGAAGG | GCGT CTC GAG GAA CATGCCCCCTAA |
| CP6727P | GCGTC CCG GGT CATATGCTACATCCACTAATGGC | GCGT CTC GAG GAAAGAATAACGAGTTCC |
| CP6729P | GCGTC CCG GGT CAT ATGGCAGATGCTTCTTTATC | GCGT CTC GAG GAATGAGTATCTTAGCC |
| CP6731P | GCGTC CCG GGT CATATGGCTGTTGTTGAAATCAAT | GCGTC CAT GGC GGC CGC GAACTGGAACTTACCTCC |
| CP6736P | GCGTC CCG GGT GCT AGCGTAGAAGTTATCATGCCTT | GCGTC CAT GGC GGC CGC AAATCGTAATTTGCTTC |
| CP6737P | GCGT GGA TCC CAT ATG GAGACTAGACTCGGAGG | GCGT CTC GAG AAATGTGGATTTTAGTCC |
| CP6751P | GCGTC CCG GGT GCT AGC AATGAAGGTCTCCAACT | GCGT CTC GAG AAATCTCATTCTACTCGC |
| CP6752P | GCGTGA ATT CAT ATGTTCGGGATGACTCCT | GCGT CTC GAG GAATTTTAAGGTACTTCCTG |
| CP6753P | GCGTC CCG GGT GCT AGCACTCCCTACTCTCATAGAG | GCGT CTC GAG AAACTTAAAGGTCGTTC |
| CP6767P | GCGTC CCG GGT CAT ATG ATAAAACAAATAGGCCGT | GCGT CTC GAG TTCGTAAGCAACTTCAGA |
| CP6829P | GCGTC CCG GGT CAT ATG AAGCAGATGCGTCTTT | GCGTC CAT GGC GGC CGC GAAACTAAGGGAGAGGC |
| CP6830P | GCGTC CCG GGT CAT ATG GATCCCGCGTCTGTT | GCGTC CAT GGC GGC CGC GAATACAAACCGGATCC |
| CP6832P | GCGTC CCG GGT CAT ATG CATAAAGTAATAGTTTTCATTT | GCGT CTC GAG TAAACTAGAAAAAGTCGTC |
| CP6848P | GCGTC CCG GGT CAT ATG TCATCAAATCTACATCCC | GCGT CTC GAG AACGCGAGCTATTTTAC |
| CP6849P | GCGTC CCG GGT GCT AGC AGCGGGGGTATAGAG | GCGT CTC GAG ATACACGTGGGTATTTTC |
| CP6850P | GCGTC CCG GGT CAT ATG TGCCGCATTGTAGAT | GCGT CTC GAG CTGTTTGCATCTGCC |
| CP6854P | GCGTC CCG GGT GCT AGC TCAATAGCTATTGCAAG | GCGT CTC GAG TTATCGAAATGTCTTTG |
| CP6879P | GCGTC CCG GGT CAT ATG GCAACACCCGCTCAA | GCGTC CAT GGC GGC CGC TCCTTGAAATTGCTCTTGC |
| CP6894P | GCGTC CCG GGT CAT ATG TATAAAAGATGTGTGCTAGA | GCGT CTC GAG GGATGTACTTAAGCACG |
| CP6900P | GCGTC CCG GGT CAT ATG AAGATAAAATTTTCTTGGAAG | GCGT AAG CTT GGGAAGACGATACCG |
| CP6952P | GCGTC CCG GGT CAT ATG CTCTCGGATCAATATATAGG | GCGT CTC GAG TCGAATTTCTTTTTTAGC |
| CP7034P | GCGTC CCG GGT CAT ATG AAAAAACAGGTATATCAATG | GCGT AAG CTT AAACGCTGAAATTATACC |
| CP7090P | GCGTC CCG GGT CAT ATG TGTAGCCTTTCCCCT | GCGT CTC GAG GCGTGCATGAATCTTA |
| CP7091P | GCGTC CCG GGT CAT ATG GAAGAATTAGAAGTTGTTGT | GCGT CTC GAG TAGTGTTCTCTTTATCGGT |
| CP7170P | GCGTC CCG GGT CAT ATG CTAGGGGCTGGAAACC | GCGT AAG CTT AAACTGCAGACCTGACG |
| CP7228P | GCGTC CCG GGT CAT ATG ACTGCTGTTCTTATTCTTACA | GCGT CTC GAG ATCTGAAAGCGGAGG |
| CP7249P | GCGTC CCG GGT CAT ATG ATCCCATCCCCTACC | GCGT CTC GAG ATCAGGTTGCTGAGACTT |
| CP7250P | GCGTC CCG GGT CAT ATG AATCTTTCAAACAGGTCT | GCGT CTC GAG ATTTTTTCTAGAGAGACTCTC |
| CP0018P | GTGCGT CATATG GCAACCACTCCACTAA | ACTCGCTA GCGGCCGC TAATGAGGTCCCCAG |
| CP6270P | GTGCGT CATATG AATTTATTAGGAGCTGCT | ACTCGCTA GCGGCCGC AAATTTGATTTTGCTACC |
| CP6735P | GTGCGT CATATG GCAGCACAAGTTGTATAT | ACTCGCTA GCGGCCGC TGGCGTAGAAGTGATC |
| CP6998P | GTGCGT CATATG TTGCCTGTAGGGAAC | ACTCGCTA GCGGCCGC GAATCTGAACTGACCAGA |
| CP7033P | GTGCGT CATATG GTTAATCCTATTGGTCCA | ACTCGCTA GCGGCCGC TTGGAGATAACCAGAATATA |
| CP7287P | GTGCGT CATATG TTACACAGCTCAGAACTAGA | ACTCGCTA GCGGCCGC GAAAATAATACGGATACCA |
| CP0010P | GTGCGT CATATG GCAACTGCTGAAAATATA | GCGT CTCGAG GAATTGGAACTTACCC |
| CP0468P | GTGCGT GCTAGC ATTTTTTATGACAAACTCTAT | GCGT CTCGAG AAATGTGCAATGACTCT |
| CP6272P | GTGCGT CATATG TTGACTCATCAAGAGGCT | GCGT CTCGAG GAAGGGAGGTTTTTTAGGT |
| CP6273P | GTGCGT CATATG ACATATCTGGAAGCTC | ACTCGCTA GCGGCCGC CTCCACAATTTTTATG |
| CP6362P | GTGCGT CATATG CCCTTTGATATTACTTATTATACA | GCGT CTCGAG TCGTTTCCAAATCCA |
| CP6372P | GTGCGT CATATG AAACAACACTATTCTCTAAATA | GCGT CTCGAG TTTCTTGTGGTTTTTCT |
| CP6390P | GTGCGT CATATG CGAGAGGTGCCTAAG | ACTCGCTA GCGGCCGC TCTCCTAGACAGCCTT |
| CP6402P | GTGCGT CATATG AATGTTGCGGATCTCCTTT | GCGT CTCGAG GAAGGGGTTGGCCGT |
| CP6446P | GTGCGT CATATG TGTAATCAAAAGCCCTCTT | GCGT CTCGAG GGGCTGAGGAGGAAC |
| CP6520P | GTGCGT GCTAGC AAACACTACCTATCATTTTCT | GCGT CTCGAG CAGAAAGGCTTTTCTTT |
| CP6577P | GTGCGT CATATG AATTTAGGCTATGTTAATTTA | GCGT CTCGAG GTTTTGTTTTTTGAAAGA |
| CP6602P | GTGCGT CATATG GCAGCATCAGGAGGCA | GCGT CTCGAG TGACCAAGGATAGGGTTTAG |
| CP6607P | GTGCGT CATATG CCTCGTGGTGACACTTT | GCGT CTCGAG CGCTGCTTCTTGCTC |
| CP6615P | GTGCGT CATATG TGCTCTCAAAAAACGACAA | GCGT CTCGAG TGAAGAGGCGCCATC |
| CP6624P | GTGCGT CATATG GATGCGAAAATGGGA | GCGT CTCGAG TCTTTGACATTCAAGAGC |
| CP6672P | GTGCGT CATATG ATTCCTACCATGTTAATG | GCGT CTCGAG GTCATACAATTTCCTTATATA |
| CP6679P | GTGCGT CATATG TGCACTCACTTAGGCT | GCGT CTCGAG CGAGTAGTTAGCACAAAC |
| CP6717P | GTGCGT GCTAGC AAGACAATCGTAGCTTCA | ACTCGCTA GCGGCCGC GGCTGGCATATAGGT |
| CP6784P | GTGCGT GCTAGC AAATCAAGATGTTCTATTGATA | GCGT CTCGAG TCCAAAACAACCCTCT |
| CP6802P | GTGCGT CATATG TGCGTAAGTTATATTAATTCCTT | GCGT CTCGAG CAGTCGGGCTTGTTG |
| CP6847P | GTGCGT CATATG TCGGATCTTTTACGAG | GCGT CTCGAG TTTCCTACACTGTTGTAATAAA |
| CP6884P | GTGCGT CATATG AATCAGCTGCTTTCT | GCGT CTCGAG AGAGAAGGTAATTGTACC |
| CP6886P | GTGCGT CATATG TGTCTACTTATTATCTATCTCTAC | GCGT CTCGAG TTCAGAAAAATGGCT |
| CP6890P | GTGCGT CATATG TCCCCACGACGACAA | GCGT CTCGAG TCCTGCAGCATTTAGC |
| CP6960P | GTGCGT CATATG TGTGACGTACGGTCTA | ACTCGCTA GCGGCCGC TTCACCTTGATTTCCT |
| CP6968P | GTGCGT CATATG TGCGATGCAAAAC | ACTCGCTA GCGGCCGC GGAAGTATGCTTAGATATT |
| CP6969P | GTGCGT CATATG TGCTGTGGTTACTCTATT | ACTCGCTA GCGGCCGC AAAAAGGTCATAGTATACCT |
| CP7005P | GTGCGT CATATG AAAACTGTGATATTGAACA | GCGT CTCGAG CTGAGCTTCTATTTCTATTAT |
| CP7072P | GTGCGT CATATG CCCATTTATGGGAAA | GCGT CTCGAG GTTGAGCAAAGGTTTG |
| CP7101P | GTGCGT CATATG TATTCGTGTTACAGCAA | GCGT CTCGAG GAAAAATTCTTTAGGGAG |
| CP7102P | GTGCGT CATATG GCCGCTAAAGCAAAT | GCGT CTCGAG TGAAAATGAAAGGATGGT |
| CP7105P | GTGCGT GCTAGC AGTCTATATCAAAAATGGTG | GCGT CTCGAG ATCTTTCATTTGGTTATCT |
| CP7106P | GTGCGT CATATG AAAGATTTGGGGACTCT | GCGT CTCGAG GAATCCTAAGGCATACCTA |
| CP7107P | GTGCGT GCTAGC AGTATAGTCAGAAATTCTGCA | GCGT CTCGAG GAAGCTAAGATTATAGCTACTTT |
| CP7108P | GTGCGT GCTAGC GCGGCCCTTTCCA | ACTCGCTA GCGGCCGC TTTATGTATATGGAACAGATAGG |
| CP7109P | GTGCGT CATATG GGACATTTTATTGATATTG | ACTCGCTA GCGGCCGC ATCATCAAGGTAGATAAAG |
| CP7110P | GTGCGT CATATG GGTTATTGCTATGTAATTACA | GCGT CTCGAG TTCTGATTGGACTCCA |
| CP7127P | GTGCGT CATATG GTGGCTTTAACGATAGC | ACTCGCTA GCGGCCG GCAGCCATCGTATTC |
| CP7130P | GTGCGT CATATG TTCAATATGCGAGG | GCGT CTCGAG CTTCTTATTTGAACTTTG |
| CP7140P | GTGCGT CATATG ACAGCCGGAGCAGCT | GCGT CTCGAG AGCACCCTCAATTTCATTG |
| CP7182P | GTGCGT CATATG GGATATGTTTTCTATGTGATC | GCGT CTCGAG GCTACTAAATCGAATCGA |
| CP6262P | GTGCGT CATATG ATCCCTGGATTAAGTTCA | ACTCGCTA GCGGCCGC TTCACTGGGAGCTTGA |
| CP6269P | GTGCGT CATATG TACCAGGAGAATCTAAGAT | ACTCGCTA GCGGCCGC GATTTTCTTCTTCAGCTC |
| CP6296P | GTGCGT CATATG GAGGAGGTGTCTGAGTAT | ACTCGCTA GCGGCCGC ATGTTTCTTTTTACTCTTTCT |
| CP6419P | GTGCGT CATATG GCTCCAGTCCGTGTT | GCGT CTCGAG AAGTGTTCGTTGGAAGT |
| CP6601P | GTGCGT CATATG AATAAGCTACTCAATTTCGT | GCGT CTCGAG GAAAATCTGAATTCTTCCT |
| CP6639P | GTGCGT CATATG TTAAATTCAAGCAATTCA | GCGT CTCGAG AGGAACTAAAACCTCATCT |
| CP6664P | GTGCGT GCTAGC GTTTTATTTCATGCTCAA | ACTCGCTA GCGGCCGC CTTAGAAAGACTATTTTCTAAGTA |
| CP6696P | GTGCGT CATATG TGCGTGATAATGGG | GCGT CTCGAG ATTCATCTTCGTAAAGAAT |
| CP6757P | GTGCGT CATATG GCAGTTGGTGGCGT | ACTCGCTA GCGGCCGC CTGTCCCTCTGGAGC |
| CP6790P | GTGCGT GCTAGC AGTGAACACAAAAAATCA | ACTCGCTA GCGGCCGC CTTATCGTCGTTATCAATA |
| CP6814P | GTGCGT CATATG CATGACGCACTTCTAAG | GCGT CTCGAG TACAGCTGCGCGA |
| CP6834P | GTGCGT CATATG GTTATGGGAACCTATATCG | GCGT CTCGAG TACATTTGTATTGATTTCAG |
| CP6878P | GTGCGT CATATG AACGTCCCTGATTCC | GCGT CTCGAG GCTAGCGGCTCTTTC |
| CP6892P | GTGCGT CATATG CAGAAGCATCCTTCCT | ACTCGCTA GCGGCCGC TCCTCTTTAGGAAATGG |
| CP6909P | GTGCGT CATATG TCCTCTTTAGGAAATGG | GCGT CTCGAG CAGTGCCAAGTAGGGA |
| CP7015P | GTGCGT CATATG GCAGTACGATTAATTGTTG | GCGT CTCGAG TTTATTGTAGTCTATTTTATATTTC |
| CP7035P | GTGCGT GCTAGC AGCAGAAAAGACAATGA | GCGT CTCGAG ATTTTGAGTGTCTTGCA |
| CP7073P | GTGCGT CATATG ATTACCATAAATCACGTG | GCGT CTCGAG TATCCATCGACTTATAGC |
| CP7085P | GTGCGT GCTAGC TGTATTTTCCCTTACGTA | ACTCGCTA GCGGCCGC GGATTCTGCATACTCTG |
| CP7092P | GTGCGT CATATG TCTCCTCTTCCTAAAAAA | GCGT CTCGAG GGATTCATTACTGACCA |
| CP7093P | GTGCGT CATATG AAATACCGTTCACG | GCGT CTCGAG ATTCTGTAGGGCTACGT |
| CP7094P | GTGCGT CATATG GTACACTTCTCTCATAACCC | GCGT CTCGAG TAAGTTTGTATTGCGGTAT |
| CP7132P | GTGCGT CATATG TTGTTATTAGGGACTTTAGGA | GCGT CTCGAG TTTCCCAACCGCA |
| CP7133P | GTGCGT CATATG GCTGCGAATGCTC | GCGT CTCGAG TAATTCAATACTCTTTGAAGG |
| CP7177P | GTGCGT CATATG CCTACTCAAGTTAAAACAGA | GCGT CTCGAG AAGTTTATATTTCAGCACTT |
| CP7184P | GTGCGT GCTAGC CATATAGGATTTTGCCA | GCGT CTCGAG GTACTTAGCAAAGCGAT |
| CP7206P | GTGCGT GCTAGC AAGAAGCTATATCACCCTA | GCGT CTCGAG CACACCGAGGAAAC |
| CP7222P | GTGCGT CATATG GTAGTTTCAGAAGAAAAAAGTC | GCGT CTCGAG ACGTATGCGCAACTG |
| CP7223P | GTGCGT CATATG GAAGTATTAGACCGCTCT | GCGT CTCGAG CGAGAAAAAGCTTCC |
| CP7224P | GTGCGT CATATG ATGAAGAAAATTCGAAA | ACTCGCTA GCGGCCGC TAAGCATTCACAAATGA |
| CP7225P | GTGCGT CATATG CATATTTTGCTTGATCGT | GCGT CTCGAG TCTTTTAACTAAATCTTGTTCTT |
| CP7303P | GTGCGT CATATG CTTGTCTATTGTTTTGATCC | GCGT CTCGAG AAAATATACGGAACTCGC |
| CP7304P | GTGCGT GCTAGC GAAGTTTATAGTTTTTCCC | GCGT CTCGAG TTTTTGATTCCTTAAGAAG |
| CP7305P | GTGCGT CATATG GAAGTTTATAGTTTTCACCCT | GCGT CTCGAG ACTCCTTGAGAAGGGAA |
| CP7307P | GTGCGT CATATG CTTAATCATGCTAAAAAGC | ACTCGCTA GCGGCCGC CTCTTTTATTTTAGGAAGCT |
| CP7342P | GTGCGT CATATG AAAAAAAAATTTATTTTCTACT | ACTCGCTA GCGGCCGC CACACTCTGTTCTTCTG |
| CP7347P | GTGCGT CATATG TTTTCTAAGGATTTGACTAA | GCGT CTCGAG CGAAGCAGAAGTCGT |
| CP7353P | GTGCGT CATATG AATATGCCTGTTCCTTCT | GCGT CTCGAG GGGGCGTAGGTTGTA |
| CP7193P | GTGCGT CATATG TGTTCCCTGGATCCT | ACTCGCTA GCGGCCGC AGTTATCACTATATCCACAAG |
| CP7248P | GTGCGT GCTAGC CTTGAACATTCTAAACAAGAT | GCGT CTCGAG ACGTAGTTTAAGAGCAGACT |
| CP7261P | GTGCGT CATATG TGTCTATCTGCCTACATAG | GCGT CTCGAG TTTTGATGCTTCTTTCA |
| CP7280P | GTGCGT CATATG GACCAGAAAATTGAAAA | GCGT CTCGAG AGAGGTCTTCTGAGTGC |
| CP7302P | GTGCGT CATATG AATTTCCATTGTAGTGTAGT | GCGT CGCGAG GAACAGTTCGATTTGTG |
| CP7306P | GTGCGT CATATG CTTCCTTTATCAGGGCA | ACTCGCTA GCGGCCGC TTCTTCAGGTTTCAGG |
| CP7367P | GTGCGT GCTAGC CGTTATGCCGAGGTC | GCGT CTCGAG TTCGTGCATTTGGTG |
| CP7408P | GTGCGT CATATG TTGAAAATCCAGAAAAA | GCGT CTCGAG ATTCATTTTCGGAAGAG |
| CP7409P | GTGCGT CATATG AGACGTTATCTTTTCATGGT | GCGT CTCGAG CCCTTTGCTCTTTACATAG |
| CP6733P | GTGCGT ACTAGT TGTCACCTACAGTCACTAG | GCGT CTCGAG GAATCGGAGTTTGGTA |
| CP6728P | GTGCGT ACTAGT AAGTCCTCTGTCTCTTGG | GCGT CTCGAG GAAACAAAACTTAGAGCCC |

TABLE III
Proteins with best results in FACS analysis

Molecular Weight (kDa)

| cp number | Theoretical | Western Blot | Fusion type |
|---|---|---|---|
| 6260 | 97.5 | 94; 70 | GST |
| 6270 | 87.5 | — | GST |
| 6272 | 78.0 | 90 | GST |
| 6273 | 58.6 | 74; 64; 50 | GST |
| 6296 | 31.1 | — | GST |
| 6390 | 88.9 | 102 | GST |
| 6456 | 42.5 | 89; 67, 45 | GST |
| 6466 | 57.5 | 59; 56 | His |
| 6467 | 59.0 | 67 | GST |
| 6552 | 28.4 | 50; 27 | GST |
| 6576 | 86.0 | 79; 70; 62; 45 | GST |
| 6577 | 17.3 | 12 | GST |
| 6602 | 43.4 | 53; 42; 34 | GST |
| 6664 | 54.5 | 104; 45 | GST |
| 6696 | 47.9 | 95; 53 | GST |
| 6727 | 130.0-142.9 | 123; 61; 39 | His |
| 6729 | 94.8 | multiple bands | GST |
| 6731 | 95.5 | 97 | GST |
| 6733 | 97.1 | 104 | His |
| 6736 | 100.1 | 98; 93; 66; 60 | GST |
| 6737 | 101.2 | multiple bands | GST |
| 6751 | 100.2 | 95; 71 | GST |
| 6752 | 102.1 | 97; 48 | His |
| 6767 | 29.1 | 28 | GST |
| 6784 | 32.9 | 35 | GST |
| 6790 | 71.3 | multiple bands | His |
| 6802 | 29.7 | — | GST |
| 6814 | 29.6 | 28 | GST |
| 6830 | 177.4 | 174; 91; 13 | GST |
| 6849 | 57.3 | multiple bands | GST |
| 6850 | 7.4-9.4 | 61; 14; 8 | GST |
| 6854 | 42.2 | — | GST |
| 6878 | 40.4 | — | GST |
| 6900 | 28.0 | — | GST |
| 6960 | 25.6 | 75; 35 | GST |
| 6968 | 34.6 | 83; 53; 35 | GST |
| 6998 | 39.3 | multiple bands | GST |
| 7033 | 68.2 | multiple bands | GST |
| 7101 | 113 | 105 | GST |
| 7102 | 63.4 | — | GST |
| 7105 | 29.2 | 30 | GST |
| 7106 | 39.5 | 72; 46 | GST |
| 7107 | 71.4 | 67; 31 | His |
| 7108 | 35.9 | 35 | GST |
| 7111 | 46.1 | 51 | GST |
| 7132 | 17.9 | 57; 47; 17 | His |
| 7140 | 36.2-29.8 | 50; 38; 34 | GST |
| 7170 | 34.4 | 77; 33 | GST |
| 7224 | 39.4 | 40 | GST |
| 7287 | 167.3 | 180 | GST |
| 7306 | 50.1 | 50 | GST |

TABLE IV
FACS-positive proteins not found in C. trachomatis

| | |
|---|---|
| cp7105 | cp6390 |
| cp7106 | cp6784 |
| cp7107 | cp6296 |
| cp7108 | |

TABLE V
Proteins identified by MALDI-TOF following 2D electrophoresis
cp6270
cp6552
cp6576
cp6577
cp6602
cp6664
cp6727
cp6728
cp6729
cp6733
cp6736
cp6737
cp6752
cp6767
cp6784
cp6790
cp6830
cp6849
cp6900
cp6960
cp6998
cp7033
cp7108
cp7111
cp7170
cp7287
cp7306

1. A protein comprising an amino acid sequence selected from the group consisting of SEQ IDs 97, 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, & 377.

2. A protein having 50% or greater sequence identity to a protein according to claim 1.

3. A protein comprising a fragment of an amino acid sequence selected from the group consisting of SEQ IDs 97, 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, & 377.

4. A nucleic acid molecule which encodes a protein according to any one of claims 1 to 3.

5. A nucleic acid molecule according to claim 4, comprising a nucleotide sequence selected from the group consisting of SEQ IDs 98, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, & 378.

6. A nucleic acid molecule comprising a fragment of a nucleotide sequence selected from the group consisting of SEQ IDs 98, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, & 378.

7. A nucleic acid molecule comprising a nucleotide sequence complementary to a nucleic acid molecule according to any one of claims 4 to 6.

8. A nucleic acid molecule comprising a nucleotide sequences having 50% or greater sequence identity to a nucleic acid molecule according to any one of claims 4 to 7.

9. A nucleic acid molecule which can hybridise to a nucleic acid molecule according to any one of claims 4 to 8 under high stringency conditions.

10. A composition comprising a protein or a nucleic acid molecule according to any preceding claim.

11. A composition according to claim 10 being a vaccine composition.

12. A composition according to claim 10 or claim 11 for use as a pharmaceutical.

13. The use of a composition according to claim 10 in the manufacture of a medicament for the treatment or prevention of infection due to Chlamydia bacteria, particularly Chlamydia pneumoniae.
